(19) United States
(12) Patent Application Publication
(10) Pub. No.: US 2023/0010108 A1
(43) Pub. Date: Jan. 12, 2023

(54) A CONJUGATION LINKER CONTAINING 2,3-DIAMINOSUCCINYL GROUP

(71) Applicant: Hangzhou DAC Biotech Co., Ltd., Hangzhou, Zhejiang (CN)

(72) Inventors: Robert Yongxin ZHAO, Lexington, MA (US); Qingliang YANG, Hangzhou, Zhejiang (CN); Yuanyuan HUANG, Hangzhou, Zhejiang (CN); Linyao ZHAO, Hangzhou, Zhejiang (CN); Hangbo YE, Hangzhou, Zhejiang (CN); Xiaotao ZHUO, Hangzhou, Zhejiang (CN); Chengyu YANG, Hangzhou, Zhejiang (CN); Jun LEI, Hangzhou, Zhejiang (CN); Yifang XU, Hangzhou, Zhejiang (CN); Huihui GUO, Hangzhou, Zhejiang (CN); Wenjun LI, Hangzhou, Zhejiang (CN); Shun GAI, Hangzhou, Zhejiang (CN); Lu BAI, Hangzhou, Zhejiang (CN); Zhixiang GUO, Hangzhou, Zhejiang (CN); Junxiang JIA, Hangzhou, Zhejiang (CN); Jun ZHENG, Hangzhou, Zhejiang (CN); Xiaomai ZHOU, Hangzhou, Zhejiang (CN); Hongsheng XIE, Hangzhou, Zhejiang (CN); Qianqian TONG, Hangzhou, Zhejiang (CN); Mingjun CHAO, Hangzhou, Zhejiang (CN); Yanhong TONG, Hangzhou, Zhejiang (CN); Zhicang YE, Hangzhou, Zhejiang (CN); Chen LIN, Hangzhou, Zhejiang (CN); Yanlei YANG, Hangzhou, Zhejiang (CN); Binbin CHEN, Hangzhou, Zhejiang (CN)

(73) Assignee: Hangzhou DAC Biotech Co., Ltd., Hangzhou, Zhejiang (CN)

(21) Appl. No.: 17/284,091

(22) PCT Filed: Oct. 12, 2018
(86) PCT No.: PCT/CN2018/110155

§ 371 Date: Apr. 9, 2021

(51) Int. Cl. C07D 403/12; C07D 417/12; C07D 413/14; C07D 417/14; C07D 403/14; C07D 498/22; C07D 498/12; A61K 47/54; A61K 45/06; A61K 47/68

(52) U.S. Cl. CPC C07D 403/12; C07D 417/12; C07D 413/14; C07D 417/14; A61K 47/6801; C07D 498/22; C07D 498/12; A61K 47/545; A61K 45/06; C07D 403/14

(57) ABSTRACT

The present invention relates to a conjugate of a cytotoxic drug/molecule to a cell-binding molecule with a bis-linker (a dual-linker) containing a 2,3-diaminosuccinyl group. It also relates to preparation of the conjugate of a cytotoxic drug/molecule to a cell-binding molecule with the bis-linker, particularly when the drug having functional groups of amino, hydroxyl, diamino, amino-hydroxyl, dihydroxyl, carboxyl, hydrazine, aldehyde and thiol for conjugation with the bis-linker in a specific manner, as well as the therapeutic use of the conjugates.

### FIELD OF THE INVENTION

The present invention relates to a conjugate of a cytotoxic drug/molecule to a cell-binding molecule with a bis-linker (a dual-linker) containing a 2,3-diaminosuccinyl group. It also relates to preparation of the conjugate of a cytotoxic drug/molecule to a cell-binding molecule with the bis-linker, particularly when the drug having functional groups of amino, hydroxyl, diamino, amino-hydroxyl, dihydroxyl, carboxyl, hydrazine, aldehyde and thiol for conjugation with the bis-linker in a specific manner, as well as the therapeutic use of the conjugates.

### BACKGROUND OF THE INVENTION

An antibody-drug conjugate (ADC), which is synergistic combination of an antibody (mAbs) conjugated to small-molecule chemotherapeutics, via a conditionally stable linker for preferential accumulation of the small-molecule drugs within the tumor through receptor-mediated endocytosis and thus sparing healthy tissue, has given rise to an extremely efficacious class of anti-cancer drugs with an already large and rapidly growing clinical pipeline. The three components of ADCs (mAb, linker and cytotoxin) affect the efficacy and toxicity of the conjugate. Optimizing each one, while enhancing the functionality of the ADC as a whole, has been one of the major considerations of ADC design and development. It is believed that the linker technology to achieve release at the desired site, efficient drug loading, optimum stoichiometry and homogeneity of the macromolecule are vitally important for attaining good pharmacokinetics, efficacy, and tolerability (Lambert, J. and Chari, R., J. Med. Chem. 2014, 57, 6949-64; Ponte, J. et al., Bioconj. Chem., 2016, 27(7), 1588-98; Dovgan, I., et al. Sci. Rep. 2016, 6, 30835; Ross, P. L. and Wolfe, J. L. J. Pharm. Sci. 105(2), 391-7; Chen, T. et al. J. Pharm. Biomed. Anal., 2016, 117, 304-10; Zhao, R. Y. et al, 2011, J. Med. Chem. 54, 3606-23).

Previous investigations on antibody-drug conjugate (ADC) stability have focused on drug release by linker-deconjugation on ADC stability due to the relatively stable payloads such as maytansines (Piwko C, et al, Clin Drug Investig. 2015, 35(8), 487-93; Lambert, J. and Chari, R., J. Med. Chem. 2014, 57, 6949-64). But, a commercial available antibody-maytansine conjugate, called T-DM1, had failed in clinic trial as first-line treatment for patients with HER2 positive unresectable locally advanced or metastatic breast cancer and as the second line treatment of HER2-positive advanced gastric cancer due to a little benefit to patients when comparison the side toxicity to the efficacy (Ellis, P. A., et al, J. Clin. Oncol. 2015, 33, (suppl; abstr 507 of 2015 ASCO Annual Meeting); Shen, K. et al, Sci Rep. 2016; 6: 23262; de Goeij, B. E. and Lambert, J. M. Curr Opin Immunol 2016, 40, 14-23; Barrios, C. H. et al, J Clin Oncol 2016, 34, (suppl; abstr 593 of 2016 ASCO Annual Meeting).

To address issues of the off-target toxicity, research and development into ADC chemistry and design are now expanding the scopes of the linker-payload compartments and conjugate chemistry beyond the sole potent payloads, and especially to address activity of the linker-payload of ADCs toward targets/target diseases (Lambert, J. M. Ther Deliv 2016, 7, 279-82; Zhao, R. Y. et al, 2011, J. Med. Chem. 54, 3606-23). Nowadays many drug developers and academic institutions are highly focusing on establishing novel reliable specific conjugation linkers and methods for site-specific ADC conjugation, which seem to have longer circulation half-life, higher efficacy, potentially decreased off-target toxicity, and a narrow range of in vivo pharmacokinetic (PK) properties of ADCs as well as better batch-to-batch consistency in ADC production (Hamblett, K. J. et al, Clin. Cancer Res. 2004, 10, 7063-70; Adem, Y. T. et al, Bioconjugate Chem. 2014, 25, 656-664; Boylan, N. J. Bioconjugate Chem. 2013, 24, 1008-1016; Strop, P., et al 2013 Chem. Biol. 20, 161-67; Wakankar, A. mAbs, 2011, 3, 161-172). These specific conjugation methods reported so far include incorporation of engineered cysteines (Junutula, J. R. et al. Nat. Biotechnol. 2008, 26, 925-32; Junutula, J. R., et al 2010 Clin. Cancer Res. 16, 4769; U.S. Pat. Nos. 8,309,300; 7,855,275; 7,521,541; 7,723,485, WO2008/141044), selenocysteines (Hofer, T., et al. Biochemistry 2009, 48, 12047-57; Li, X., et al. Methods 2014, 65, 133-8; U.S. Pat. No. 8,916,159 for US National Cancer Institute), cysteine containing tag with perfluoroaromatic reagents (Zhang, C. et al. Nat. Chem. 2015, 8, 1-9), thiolfucose (Okeley, N. M., et al 2013 Bioconjugate Chem. 24, 1650), non-natural amino acids (Axup, J. Y., et al, Proc. Nat. Acad. Sci. USA. 2012, 109, 16101-6; Zimmerman, E. S., et al., 2014, Bioconjug. Chem. 25, 351-361; Wu, P., et al, 2009 Proc. Natl. Acad. Sci. 106, 3000-5; Rabuka, D., et al, Nat. Protoc. 2012, 7, 1052-67; U.S. Pat. No. 8,778,631 and US Pat Appl. 20100184135, WO2010/081110 for Sutro Biopharma; WO2006/069246, 2007/059312, U.S. Pat. Nos. 7,332,571, 7,696,312, and 7,638,299 for Ambrx; WO2007/130453, U.S. Pat. Nos. 7,632,492 and 7,829,659 for Allozyne), conjugation to reduced intermolecular disulfides by re-bridging dibromomalemides (Jones, M. W. et al. J. Am. Chem. Soc. 2012, 134, 1847-52), bis-sulfone reagents (Badescu, G. et al. Bioconjug. Chem. 2014, 25, 1124-36; WO2013/190272, WO2014/064424 for PolyTherics Ltd). dibromopyridazinediones (Maruani, A. et al. Nat. Commun. 2015, 6, 6645), galactosyl- and sialyltransferases (Zhou, Q. et al. Bioconjug. Chem. 2014, 25, 510-520; US Pat Appl 20140294867 for Sanofi-Genzyme), formylglycine generating enzyme (FGE) (Drake, P. M. et al. Bioconj. Chem. 2014, 25, 1331-41; Carrico, I. S. et al U.S. Pat. Nos. 7,985,783; 8,097,701; 8,349,910, and US Pat Appl 20140141025, 20100210543 for Redwood Bioscience), phosphopantetheinyl transferases (PPTases) (Grunewald, J. et al. Bioconjug. Chem. 2015, 26, 2554-62), sortase A (Beerli, R. R., et al. PLoS One 2015, 10, e0131177), genetically introduced glutamine tag with Streptoverticillium mobaraense transglutaminase (mTG) (Strop, P., Bioconj. Chem., 2014, 25, 855-62; Strop, P., et al., Chem. Biol. 2013, 20, 161-7; U.S. Pat. No. 8,871,908 for Rinat-Pfizer) or with microbial transglutaminase (MTGase) (Dennler, P., et al, 2014, Bioconjug. Chem. 25, 569-78; Siegmund, V. et al. Angew. Chemie-Int. Ed. 2015, 54, 13420-4; US pat appl 20130189287 for Innate Pharma; U.S. Pat. No. 7,893,019 for Bio-Ker S.r.l. (IT)), an enzyme/bacterium forming an isopeptide bond-peptide bonds that form outside of the protein main chain (Kang, H. J., et al. Science 2007, 318, 1625-8; Zakeri, B. et al. Proc. Natl. Acad. Sci. USA 2012, 109, E690-7; Zakeri, B. & Howarth, M. J. Am. Chem. Soc. 2010, 132, 4526-7).

We have disclosed several conjugation methods of rebridging a pair of thiols of the reduced inter chain disulfide bonds of a native antibody, such as using bromo maleimide and dibromomaleimide linkers (WO2014/009774), 2,3-disubstituted succinic/2-monosubstituted/2,3-disubstituted fumaric or maleic linkers (WO2015/155753, WO20160596228), acetylenedicarboxylic linkers (WO2015/151080, WO20160596228) or hydrazine linkers (WO2015/151081). The ADCs made with these linkers and methods have demonstrated better therapeutic index windows than the traditionally unselective conjugation via the cysteine or lysine residues on an antibody. Here we disclose the invention of bis-linkers containing 2,3-diaminosuccinyl group and methods for conjugation of a cytotoxic molecule, particularly when the cytotoxic agent having dual groups of diamino, amino-hydroxyl, dihydroxyl, carboxyl, aldehyde, hydrazine, thiols or combination above. The immunoconjugates made with the bis-linkage have prolonged the half-life during the targeted delivery and minimized exposure to non-target cells, tissues or organs during the blood circulation, resulting in less the off-target toxicity.

### SUMMARY OF THE INVENTION

The present invention provides bis-linkage of an antibody with a cytotoxic agent, particularly when the cytotoxic agent having two functional groups of an amino, hydroxyl, diamino, amino-hydroxyl, dihydroxyl, carboxyl, hydrazine, or thiol. It also provides a bis-linker for conjugation of cell-binding molecule to a cytotoxic molecule in a specific manner.

In one aspect of the present invention, a conjugate with a bis-linkage containing 2,3-diaminosuccinyl group is represented by Formula (I), (II), (III) or (IV):

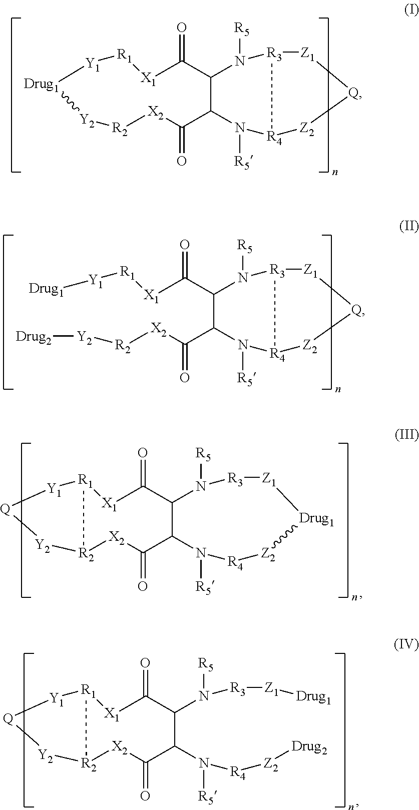

wherein

“ [symbol] ” represents a single bond;

“ [symbol] ” is optionally either a single bond, or absent;

“-----” is optionally either a single bond, or a double bond, or can optionally be absent;

n is 1 to 30 independently;

Q is a cell-binding agent/molecule that links to R₃ and R₄ can be any kind presently known, or that become known, of a molecule that binds to, complexes with, or reacts with a moiety of a cell population sought to be therapeutically or otherwise biologically modified. Preferably the cell-binding agent/molecule is an immunotherapeutic protein, an antibody, an antibody fragment, or peptides having over four amino acids;

Drug₁ or/and Drug₂ are a cytotoxic molecule/agent that is a therapeutic drug, or an immunotherapeutic protein/molecule, or a function molecule for enhancement of binding or stabilization of the cell-binding agent, or a cell-surface receptor binding ligand, or for inhibition of cell proliferation;

X₁ and X₂ are the same or different, and independently selected from NH; NHNH; N(R₁); N(R₁)N(R₂); O; S; S—S, O—NH. O—N(R₁), CH₂—NH. CH₂—N(R₁), CH═NH. CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NH)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); or C₁-C₆ alkyl; C₂-C₈ alkenyl, heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl;

Y₁, Y₂, Z₁ and Z₂ are, the same or different, and independently a function group that link to a cell-binding molecule Q, or drug₁ or drug₂, to form a disulfide, ether, ester, thioether, thioester, peptide, hydrazone, carbamate, carbonate, amine (secondary, tertiary, or quarter), imine, cycloheteroalkyane, heteroaromatic, alkyloxime or amide bond; Preferably Y₁, Y₂, Z₁ and Z₂ independently have the following structures: C(O)CH, C(O)C, C(O)CH₂, ArCH₂, C(O), NH, NHNH, N(R₁), N(R₁)N(R₂), O, S, S—S, O—NH, O—N(R₁), CH₂—NH. CH₂—N(R₁), CH═NH. CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); or C₁-C₈ alkyl, C₂-C₅ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl;

Preferably Y₁, Y₂, Z₁ and Z₂ are linked to pairs of thiols of a cell-binding agent/molecule. The thiols are preferably pairs of sulfur atoms reduced from the inter chain disulfide bonds of the cell-binding agent by a reduction agent selected from dithiothreitol (DTT), dithioerythritol (DTE), L-glutathione (GSH), tris (2-carboxyethyl) phosphine (TCEP), 2-mercaptoethylamine (β-MEA), or/and beta mercaptoethanol (β-ME, 2-ME);

R₁, R₂, R₃, and R₄ are a chain of atoms selected from C, N, O, S, Si, and P, preferably having 0-500 atoms, which covalently connects to X and Z₁, and Y and Z₂. The atoms used in forming R₁, R₂, R₃, and R₄ may be combined in all chemically relevant ways, such as forming alkylene, alkenylene, and alkynylene, ethers, polyoxyalkylene, esters, amines, imines, polyamines, hydrazines, hydrazones, amides, ureas, semicarbazides, carbazides, alkoxyamines, alkoxylamines, urethanes, amino acids, peptides, acyloxylamines, hydroxamic acids, or combination above thereof. Preferably R₁, R₂, R₃, and R₄ are, the same or different, independently selected from O, NH, S, NHNH, N(R₅), N(R₃)N(R_{3′}), polyethyleneoxy unit of formula (OCH₂CH₂)ₚOR₅, or (OCH₂CH—(CH₃))ₚOR₅, or NH(CH₂CH₂O)ₚR₅, or NH(CH₂CH(CH₃)O)ₚR₅, or N[(CH₂CH₂O)ₚR₅]—[(CH₂CH₂O)_{p′}R_{5′}], or (OCH₂CH₂)ₚCOOR₅, or CH₂CH₂(OCH₂CH₂)ₚCOOR₅, wherein p and p′ are independently an integer selected from 0 to about 1000, or combination thereof; C₁-C₈ alkyl; C₂-C₈ heteroalkyl, or alkylcycloalkyl, heterocycloalkyl; C₃-C₈ aryl, Ar-alkyl, heterocyclic, carbocyclic, cycloalkyl, heteroalkylcycloalkyl, alkylcarbonyl, or heteroaryl;

More preferably R₁, R₂, R₃, R₄, R₅ and R_{5′} are independently H; C₁-C₈ alkyl; C₂-C₈ heteroalkyl, alkylcycloalkyl, or heterocycloalkyl; C₃-C₈ aryl, Ar-alkyl, heterocyclic, carbocyclic, heteroalkylcycloalkyl, alkylcarbonyl, or heteroaryl; or C₁-C₈ carbon atoms esters, ether, or amide; or 1-24 amino acids; or polyethyleneoxy having formula (OCH₂CH₂)ₚ or (OCH₂CH(CH₃))ₚ, wherein p is an integer from 0 to about 5000, or combination above thereof;

R₁, R₂, R₃, and R₄ may optionally be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1-8 natural or unnatural amino acid unites. The natural aminoacid is preferably selected from aspartic acid, glutamic acid, arginine, histidine, lysine, serine, threonine, asparagine, glutamine, cysteine, selenocysteine, tyrosine, phenylalanine, glycine, proline, tryptophan, and alanine;

In addition, R₁, R₂, R₃, R₄, Y₁, Y₂, Z₁, and Z₂ may be independently absent.

In another aspect, this invention provides a readily-reactive bis-linker of Formula (V), (VI), (VII) and (VIII) containing 2,3-diaminosuccinyl group below, wherein two or more residues of a cell-binding molecule can simultaneously or sequentially react them to form Formula (I), (II), (III) and (IV) above:

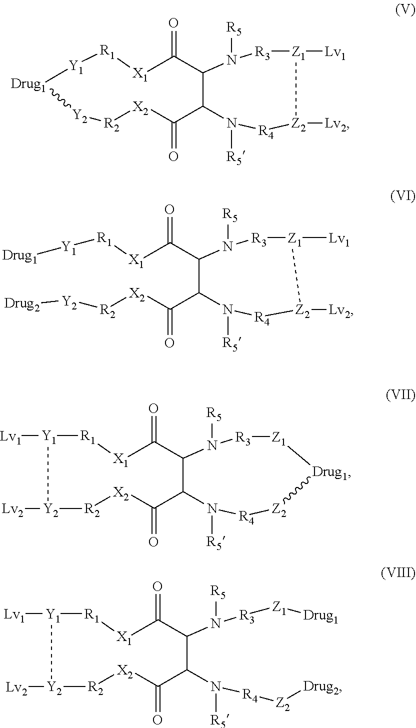

wherein.

“-----” is optionally either a single bond, or a double bond, or a triple bond, or can optionally be absent; It provided that when ----- represents a triple bond, both Lv₁ and Lv₂ are absent;

“ [symbol] ”, “ [symbol] ”, Drug₁, Drug₂, n, X₁, X₂, Y₁, Y₂, R₁, R₂, R₃, R₄, R₅, R_{5′}, Z₁, and Z₂ are defined the same as in Formula (I)-(IV);

Lv₁ and Lv₂ represent the same or different leaving group that can be reacted with a thiol, amine, carboxylic acid, selenol, phenol or hydroxyl group on a cell-binding molecule. Such leaving groups are, but are not limited to, a halide (e.g., fluoride, chloride, bromide, and iodide), methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluorophenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or carbon-oxygen), or an intermediate molecule generated with a condensation reagent for Mitsunobu reactions.

In another aspect, this invention provides a readily-reactive bis-linker of Formula (IX) and (X) of following, wherein two or more function groups of a cytotoxic molecule can react it simultaneously or sequentially to form Formula (I), (II), (III) or (IV) above.

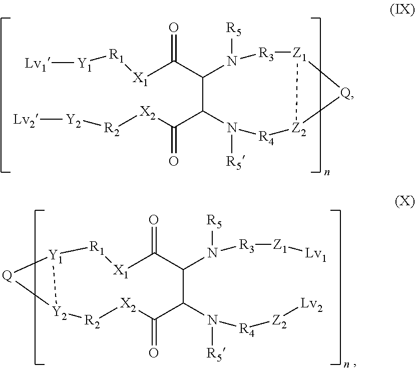

wherein:

“ [symbol] ”, “ [symbol] ”, Q, n, X₁, X₂, Y₁, Y₂, R₁, R₂, R₃, R₄, R₅, R_{5′}, Z₁, and Z₂ are defined the same as in Formula (I)-(IV); and “-----”, Lv₁, Lv₂, Lv₁′ and Lv₂′ are defined the same as Lv₁ and Lv₂ in Formula (V)-(VIII);

In another aspect, this invention provides a readily-reactive bis-linker of Formula (XI) and (XII) below, wherein a cytotoxic molecule and a cell-binding molecule can react it independently, or simultaneously, or sequentially to form Formula (I)-(IV).

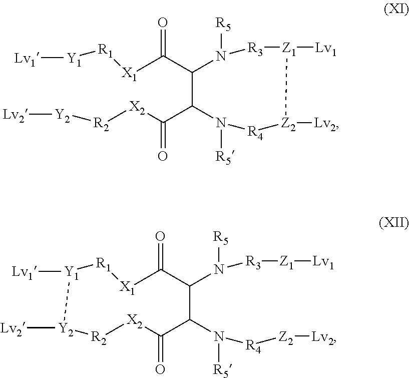

wherein “ [symbol] ”, X₁, X₂, Y₁, Y₂, R₁, R₂, R₃, R₄, R₅, R_{5′}, Z₁, and Z₂ are defined the same as in Formula (I)-(IV); and “-----”, Lv₁, Lv₂, Lv₁′ and Lv₂′ are defined the same as Lv₁ and Lv₂ in Formula (V)-(VIII);

The present invention further relates to a method of making a cell-binding molecule-drug conjugate of Formula (I), (II), (III) and (IV).

### BRIEF DESCRIPTION OF THE DRAWINGS

FIG. 1 shows the synthesis of analogs of tyrosine (Tyr) and tubutyrosine (Tut) that have an amino or nitro group on the benzene ring for bis-linked to a cell-binding molecule.

FIG. 2 shows the synthesis of components of tubulysin analogs.

FIG. 3 shows the synthesis of components of tubulysin analogs.

FIG. 4 shows the synthesis a bis-linker containing a 2,3-diaminosuccinyl group and a tubulysin analog containing a bis-linker having a 2,3-diaminosuccinyl group.

FIG. 5 shows the synthesis of a tubulysin analog with a bis-linker containing a 2,3-diaminosuccinyl group and its conjugation to an antibody via a pair of thiols of the antibody.

FIG. 6 shows the synthesis of a tubulysin analog with a bis-linker containing a 2,3-diaminosuccinyl group and its conjugation to an antibody via a pair of thiols of the antibody.

FIG. 7 shows the synthesis of a tubulysin analog with a bis-linker containing a 2,3-diaminosuccinyl group and its conjugation to an antibody via a pair of thiols of the antibody.

FIG. 8 shows the synthesis of a tubulysin analog with a bis-linker containing a 2,3-diaminosuccinyl group and its conjugation to an antibody via a pair of thiols of the antibody FIG. 9 shows the synthesis of a tubulysin analog with a bis-linker containing a 2,3-diaminosuccinyl group and its conjugation to an antibody via a pair of thiols of the antibody.

FIG. 10 shows the synthesis of a tubulysin analog with a bis-linker containing a 2,3-diaminosuccinyl group and its conjugation to an antibody via a pair of thiols of the antibody.

FIG. 11 shows the synthesis of a tubulysin analog with a bis-linker containing a 2,3-diaminosuccinyl group and its conjugation to an antibody via a pair of thiols of the antibody, and the synthesis of auristatin components.

FIG. 12 shows the synthesis of the synthesis of auristatin components containing a bis-linker.

FIG. 13 shows the synthesis of the synthesis of auristatin F containing a bis-linker and its conjugation to an antibody, and the synthesis of components of an amanitin and a linker.

FIG. 14 shows the synthesis of the synthesis of auristatin F containing a bis-linker and its conjugation to an antibody.

FIG. 15 shows the synthesis of an amanitin analog containing a bis-linker.

FIG. 16 shows the synthesis of conjugation of an amanitin analog containing a bis-linker to an antibody via a pair of thiols on the antibody.

FIG. 17 shows the synthesis of conjugation of an amanitin analog containing a bis-linker to an antibody via a pair of thiols on the antibody.

FIG. 18 shows the synthesis of conjugation of tubulysin analog and a CBI-dimer analog containing a bis-linker to an antibody via a pair of thiols of the antibody.

FIG. 19 shows the synthesis of a CBI-dimer analog containing a bis-linker and its conjugation to an antibody via a pair of thiols of the antibody.

FIG. 20 shows the synthesis of a CBI-dimer analog containing a bis-linker and its conjugation to an antibody via a pair of thiols of the antibody.

FIG. 21 shows the synthesis of a CBI-dimer analog containing a bis-linker and its conjugation to an antibody via a pair of thiols of the antibody.

FIG. 22 shows the synthesis of a CBI-dimer analog containing a bis-linker and its conjugation to an antibody via a pair of thiols of the antibody, and the synthesis of components of a PBD dimer.

FIG. 23 shows the synthesis of a PBD dimer containing a bis-linker and its conjugation to an antibody via a pair of thiols of the antibody.

FIG. 24 shows the synthesis of a PBD dimer containing a bis-linker and its conjugation to an antibody via a pair of thiols of the antibody.

FIG. 25 shows the synthesis of a PBD dimer containing a bis-linker and its conjugation to an antibody via a pair of thiols of the antibody.

FIG. 26 shows the synthesis of a PBD dimer containing a bis-linker and its conjugation to an antibody via a pair of thiols of the antibody.

FIG. 27 shows the comparison of the anti-tumor effect of conjugate compounds Ba-12, Ba-14, Ba-16, Ca-03, Ca-04, Ca-05, Ca-06, Ca-07, Ca-10, Ca-11, Ca-12, along with T-DM1 and PBS (control) using human gastric tumor N87 cell model, i.v., one injection at dosing of 3 mg/kg for conjugates. All 12 conjugates tested except Ca-06 demonstrated anti-tumor activity.

### DETAILED DESCRIPTION OF THE INVENTION

### Definitions

“Alkyl” refers to an aliphatic hydrocarbon group or univalent groups derived from alkane by removal of one or two hydrogen atoms from carbon atoms. It may be straight or branched having C₁-C₈ (1 to 8 carbon atoms) in the chain. “Branched” means that one or more lower C numbers of alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl, cyclopentyl, cyclohexyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methyl-hexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)₂R′, —S(O)R′, —OH, -halogen, —N₃, —NH₂, —NH(R′), —N(R′)₂ and —CN; where each R′ is independently selected from —C₁-C₈ alkyl and aryl.

“Halogen” refers to fluorine, chlorine, bromine or iodine atom; preferably fluorine and chlorine atom.

“Heteroalkyl” refers to C₂-C₈ alkyl in which one to four carbon atoms are independently replaced with a heteroatom from the group consisting of O, S and N.

“Carbocycle” refers to a saturated or unsaturated ring having 3 to 8 carbon atoms as a monocycle or 7 to 13 carbon atoms as a bicycle. Monocyclic carbocycles have 3 to 6 ring atoms, more typically 5 or 6 ring atoms. Bicyclic carbocycles have 7 to 12 ring atoms, arranged as a bicycle [4,5], [5,5], [5,6] or [6,6] system, or 9 or 10 ring atoms arranged as a bicycle [5,6] or [6,6] system. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl.

A “C₃-C₈ carbocycle” refers to a 3-, 4-, 5-, 6-, 7- or 8-membered saturated or unsaturated nonaromatic carbocyclic ring. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)R′, —S(O)₂R′, —OH, -halogen, —N₃, —NH₂, —NH(R′), —N(R′) 2 and —CN; where each R′ is independently selected from —C₁-C₈ alkyl and aryl.

“Alkenyl” refers to an aliphatic hydrocarbon group containing a carbon-carbon double bond which may be straight or branched having 2 to 8 carbon atoms in the chain. Exemplary alkenyl groups include ethenyl, propenyl, n-butenyl, i-butenyl, 3-methylbut-2-enyl, n-pentenyl, hexylenyl, heptenyl, octenyl.

“Alkynyl” refers to an aliphatic hydrocarbon group containing a carbon-carbon triple bond which may be straight or branched having 2 to 8 carbon atoms in the chain. Exemplary alkynyl groups include ethynyl, propynyl, n-butynyl, 2-butynyl, 3-methylbutynyl, 5-pentynyl, n-pentynyl, hexylynyl, heptynyl, and octynyl.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene, propargyl and 4-pentynyl.

“Aryl” or “Ar” refers to an aromatic or hetero aromatic group, composed of one or several rings, comprising three to fourteen carbon atoms, preferentially six to ten carbon atoms. The term of “hetero aromatic group” refers one or several carbon on aromatic group, preferentially one, two, three or four carbon atoms are replaced by O, N, Si, Se, P or S, preferentially by O, S, and N. The term aryl or Ar also refers to an aromatic group, wherein one or several H atoms are replaced independently by —R′, -halogen, —OR′, or —SR′, —NR′R″, —N═NR′, —N═R′, —NR′R″, —NO₂, —S(O)R′, —S(O)₂R′, —S(O)₂OR′, —OS(O)₂OR′, —PR′R″, —P(O)R′R″, —P(OR′)(OR″), —P(O)(OR′)(OR″) or —OP(O)(OR′)(OR″) wherein R′, R″ are independently H, alkyl, alkenyl, alkynyl, heteroalkyl, aryl, arylalkyl, carbonyl, or pharmaceutical salts.

“Heterocycle” refers to a ring system in which one to four of the ring carbon atoms are independently replaced with a heteroatom from the group of O, N, S, Se, B, Si and P. Preferable heteroatoms are O, N and S. Heterocycles are also described in The Handbook of Chemistry and Physics, 78th Edition, CRC Press, Inc., 1997-1998, p. 225 to 226, the disclosure of which is hereby incorporated by reference. Preferred nonaromatic heterocyclic include epoxy, aziridinyl, thiiranyl, pyrrolidinyl, pyrazolidinyl, imidazolidinyl, oxiranyl, tetrahydrofuranyl, dioxolanyl, tetrahydropyranyl, dioxanyl, dioxolanyl, piperidyl, piperazinyl, morpholinyl, pyranyl, imidazolinyl, pyrrolinyl, pyrazolinyl, thiazolidinyl, tetrahydrothio-pyranyl, dithianyl, thiomorpholinyl, dihydropyranyl, tetrahydropyranyl, dihydropyranyl, tetrahydropyridyl, dihydropyridyl, tetrahydropyrimidinyl, dihydrothiopyranyl, azepanyl, as well as the fused systems resulting from the condensation with a phenyl group.

The term “heteroaryl” or aromatic heterocycles refers to a 3 to 14, preferably 5 to 10 membered aromatic hetero, mono-, bi-, or multi-cyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1,2,4-thiadiazolyl, isothiazolyl, triazolyl, tetrazolyl, isoquinolyl, benzothienyl, isobenzofuryl, pyrazolyl, carbazolyl, benzimidazolyl, isoxazolyl, pyridyl-N-oxide, as well as the fused systems resulting from the condensation with a phenyl group.

“Alkyl”, “cycloalkyl”, “alkenyl”, “alkynyl”, “aryl”, “heteroaryl”, “heterocyclic” and the like refer also to the corresponding “alkylene”, “cycloalkylene”, “alkenylene”, “alkynylene”, “arylene”, “heteroarylene”, “heterocyclene” and the likes which are formed by the removal of two hydrogen atoms.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

“Heteroarylalkyl” refers to an acyclic alkyl radical in which one of the hydrogen atoms bonded to a carbon atom, typically a terminal or sp³ carbon atom, is replaced with a heteroaryl radical. Examples of heteroarylalkyl groups are 2-benzimidazolylmethyl, 2-furylethyl.

Examples of a “hydroxyl protecting group” include, methoxymethyl ether, 2-methoxyethoxymethyl ether, tetrahydropyranyl ether, benzyl ether, p-methoxybenzyl ether, trimethylsilyl ether, triethylsilyl ether, triisopropylsilyl ether, t-butyldimethylsilyl ether, triphenylmethylsilyl ether, acetate ester, substituted acetate esters, pivaloate, benzoate, methanesulfonate and p-toluenesulfonate.

“Leaving group” refers to a functional group that can be substituted by another functional group. Such leaving groups are well known in the art, and examples include, a halide (e.g., chloride, bromide, and iodide), methanesulfonyl (mesyl), p-toluenesulfonyl (tosyl), trifluoromethylsulfonyl (triflate), and trifluoromethylsulfonate. A preferred leaving group is selected from nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

The following abbreviations may be used herein and have the indicated definitions: Boc, tert-butoxy carbonyl; BroP, bromotrispyrrolidinophosphonium hexafluorophosphate; CDI, 1,1′-carbonyldiimidazole; DCC, dicyclohexylcarbodiimide; DCE, dichloroethane; DCM, dichloromethane; DEAD is diethylazodicarboxylate, DIAD, diisopropylazodicarboxylate; DIBAL-H, diisobutyl-aluminium hydride; DIPEA or DEA, diisopropylethylamine; DEPC, diethyl phosphorocyanidate; DMA, N,N-dimethyl acetamide; DMAP, 4-(N, N-dimethylamino)pyridine; DMF, N,N-dimethylformamide; DMSO, dimethylsulfoxide; DTPA is diethylenetriaminepentaacetic acid; DTT, dithiothreitol; EDC, 1-(3-dimethylaminopropyl)-3-ethylcarbodiimide hydrochloride; ESI-MS, electrospray mass spectrometry; EtOAc is ethyl acetate; Fmoc is N-(9-fluorenylmethoxycarbonyl); HATU, 0-(7-azabenzotriazol-1-yl)-N, N, N′, N′-tetramethyluronium hexafluorophosphate; HOBt, 1-hydroxybenzotriazole; HPLC, high pressure liquid chromatography; NHS, N-Hydroxysuc-cinimide; MeCN is acetonitrile; MeOH is methanol; MMP, 4-methylmorpholine; PAB, p-aminobenzyl; PBS, phosphate-buffered saline (pH 7.0-7.5); Ph is phenyl; phe is L-phenylalanine; PyBrop is bromo-tris-pyrrolidino-phosphonium hexafluorophosphate; PEG, polyethylene glycol; SEC, size-exclusion chromatography; TCEP, tris(2-carboxyethyl)phosphine; TFA, trifluoroacetic acid; THF, tetrahydrofuran; Val, valine; TLC is thin layer chromatography; UV is ultraviolet. The “amino acid(s)” can be natural and/or unnatural amino acids, preferably alpha-amino acids. Natural amino acids are those encoded by the genetic code, which are alanine, arginine, asparagine, aspartic acid, cysteine, glutamic acid, glutamine, glycine, histidine, isoleucine, leucine, lysine, methionine, phenylalanine, proline, serine, threonine, tyrosine. tryptophan and valine. The unnatural amino acids are derived forms of proteinogenic amino acids. Examples include hydroxyproline, lanthionine, 2-aminoisobutyric acid, dehydroalanine, gamma-aminobutyric acid (the neurotransmitter), ornithine, citrulline, beta alanine (3-aminopropanoic acid), gamma-carboxyglutamate, selenocysteine (present in many noneukaryotes as well as most eukaryotes, but not coded directly by DNA), pyrrolysine (found only in some archaea and one bacterium), N-formylmethionine (which is often the initial amino acid of proteins in bacteria, mitochondria, and chloroplasts), 5-hydroxytryptophan, L-dihydroxyphenylalanine, triiodothyronine, L-3,4-dihydroxyphenylalanine (DOPA), and O-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. Preferably, an amino acid mimetic is a compound that has a structure different from the general chemical structure of an alpha-amino acid but functions in a manner similar to one. The term “unnatural amino acid” is intended to represent the “D” stereochemical form, the natural amino acids being of the “L” form. When 1-8 amino acids are used in this patent application, amino acid sequence is then preferably a cleavage recognition sequence for a protease. Many cleavage recognition sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); and Bouvier et al. Meth. Enzymol. 248: 614 (1995); the disclosures of which are incorporated herein by reference. In particular, the sequence is selected from the group consisting of Val-Cit, Ala-Val, Ala-Ala, Val-Val, Val-Ala-Val, Lys-Lys, Ala-Asn-Val, Val-Leu-Lys, Cit-Cit, Val-Lys, Ala-Ala-Asn, Lys, Cit, Ser, and Glu.

The “glycoside” is a molecule in which a sugar group is bonded through its anomeric carbon to another group via a glycosidic bond. Glycosides can be linked by an O- (an O-glycoside), N- (a glycosylamine), S- (a thioglycoside), or C- (a C-glycoside) glycosidic bond. Its core the empirical formula is Cₘ(H₂O)ₙ (where m could be different from n, and m and n are <36), Glycoside herein includes glucose (dextrose), fructose (levulose) allose, altrose, mannose, gulose, iodose, galactose, talose, galactosamine, glucosamine, sialic acid, N-acetylglucosamine, sulfoquinovose (6-deoxy-6-sulfo-D-glucopyranose), ribose, arabinose, xylose, lyxose, sorbitol, mannitol, sucrose, lactose, maltose, trehalose, maltodextrins, raffinose, Glucuronic acid (glucuronide), and stachyose. It can be in D form or L form, 5 atoms cyclic furanose forms, 6 atoms cyclic pyranose forms, or acyclic form, α-isomer (the —OH of the anomeric carbon below the plane of the carbon atoms of Haworth projection), or a β-isomer (the —OH of the anomeric carbon above the plane of Haworth projection). It is used herein as a monosaccharide, disaccharide, polyols, or oligosaccharides containing 3-6 sugar units.

The term “antibody,” as used herein, refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce auto-immune antibodies associated with an autoimmune disease. The immunoglobulin disclosed herein can be of any type (e.g. IgG, IgE, IgM, IgD, IgA and IgY), class (e.g., IgG1, IgG2, IgG3, IgG4, IgA1 and IgA2) or subclass of immunoglobulin molecule. The immunoglobulins can be derived from any species. Preferably, however, the immunoglobulin is of human, murine, or rabbit origin. Antibodies useful in the invention are preferably monoclonal, and include, but are not limited to, polyclonal, monoclonal, bispecific, human, humanized or chimeric antibodies, single chain antibodies, Fv, Fab fragments, F(ab′) fragments, F(ab′)₂ fragments, fragments produced by a Fab expression library, anti-idiotypic (anti-Id) antibodies, CDR's, and epitope-binding fragments of any of the above which immunospecifically bind to cancer cell antigens, viral antigens or microbial antigens.

An “enantiomer”, also known as an “optical isomer”, is one of two stereoisomers that are mirror images of each other that are non-superposable (not identical), much as one's left and right hands are the same except for being reversed along one axis (the hands cannot be made to appear identical simply by reorientation). A single chiral atom or similar structural feature in a compound causes that compound to have two possible structures which are non-superposable, each a mirror image of the other. The presence of multiple chiral features in a given compound increases the number of geometric forms possible, though there may be some perfect-mirror-image pairs. Enantiopure compounds refer to samples having, within the limits of detection, molecules of only one chirality. When present in a symmetric environment, enantiomers have identical chemical and physical properties except for their ability to rotate plane-polarized light (+/−) by equal amounts but in opposite directions (although the polarized light can be considered an asymmetric medium). They are sometimes called optical isomers for this reason. A mixture of equal parts of an optically active isomer and its enantiomer is termed racemic and has zero net rotation of plane-polarized light because the positive rotation of each (+) form is exactly counteracted by the negative rotation of a (−) one. Enantiomer members often have different chemical reactions with other enantiomer substances. Since many biological molecules are enantiomers, there is sometimes a marked difference in the effects of two enantiomers on biological organisms. In drugs, for example, often only one of a drug's enantiomers is responsible for the desired physiologic effects, while the other enantiomer is less active, inactive, or sometimes even productive of adverse effects. Owing to this discovery, drugs composed of only one enantiomer (“enantiopure”) can be developed to enhance the pharmacological efficacy and sometimes eliminate some side effects.

Isotopes are variants of a particular chemical element which differs in neutron number. All isotopes of a given element have the same number of protons in each atom. Each atomic number identifies a specific element, but not the isotope; an atom of a given element may have a wide range in its number of neutrons. The number of nucleons (both protons and neutrons) in the nucleus is the atom's mass number, and each isotope of a given element has a different mass number. For example, carbon-12, carbon-13 and carbon-14 are three isotopes of the element carbon with mass numbers 12, 13 and 14 respectively. The atomic number of carbon is 6, which means that every carbon atom has 6 protons, so that the neutron numbers of these isotopes are 6, 7 and 8 respectively. Hydrogen atom has three isotopes of protium (¹H), deuterium (²H), and tritium (³H), which deuterium has twice the mass of protium and tritium has three times the mass of protium. Isotopic substitution can be used to determine the mechanism of a chemical reaction and via the kinetic isotope effect. Isotopic substitution can be used to study how the body affects a specific xenobiotic/chemical after administration through the mechanisms of absorption and distribution, as well as the metabolic changes of the substance in the body (e.g. by metabolic enzymes such as cytochrome P450 or glucuronosyltransferase enzymes), and the effects and routes of excretion of the metabolites of the drug. This study is called pharmacokinetics (PK). Isotopic substitution can be used to study of the biochemical and physiologic effects of drugs. The effects can include those manifested within animals (including humans), microorganisms, or combinations of organisms (for example, infection). This study is called pharmacodynamics (PD). The effects can include those manifested within animals (including humans), microorganisms, or combinations of organisms (for example, infection). Both together influence dosing, benefit, and adverse effects of the drug. isotopes can contain a stable (non-radioactive) or an unstable element. Isotopic substitution of a drug may have a different thrapeutical efficacy of the original drug.

“Pharmaceutically” or “pharmaceutically acceptable” refer to molecular entities and compositions that do not produce an adverse, allergic or other untoward reaction when administered to an animal, or a human, as appropriate.

“Pharmaceutically acceptable solvate” or “solvate” refer to an association of one or more solvent molecules and a disclosed compound. Examples of solvents that form pharmaceutically acceptable solvates include, but are not limited to, water, isopropanol, ethanol, methanol, DMSO, ethyl acetate, acetic acid and ethanolamine.

“Pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions as suitable therapeutic combinations.

As used herein, “pharmaceutical salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucuronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutical salts of the present invention can be synthesized from the parent compound which contains a basic or acidic moiety by conventional chemical methods. Generally, such salts can be prepared via reaction the free acidic or basic forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, the disclosure of which is hereby incorporated by reference.

“Administering” or “administration” refers to any mode of transferring, delivering, introducing or transporting a pharmaceutical drug or other agent to a subject. Such modes include oral administration, topical contact, intravenous, intraperitoneal, intramuscular, intralesional, intranasal, subcutaneous or intrathecal administration. Also contemplated by the present invention is utilization of a device or instrument in administering an agent. Such device may utilize active or passive transport and may be slow-release or fast-release delivery device.

“Therapeutically effective amount” means an amount of a compound/medicament according to the present invention effective in preventing or treating the herein referred pathological condition.

The term “patient”, or “patient in need thereof”, is intended for an animal or a human being affected or likely to be affected with the herein referred pathological condition. Preferably, the patient is human.

In the context of cancer, the term “treating” includes any or all of: preventing growth of tumor cells or cancer cells, preventing replication of tumor cells or cancer cells, lessening of overall tumor burden and ameliorating one or more symptoms associated with the disease.

In the context of an autoimmune disease, the term “treating” includes any or all of: preventing replication of cells associated with an autoimmune disease state including, but not limited to, cells capable of producing an autoimmune antibody, lessening the autoimmune-antibody burden and ameliorating one or more symptoms of an autoimmune disease. In the context of an infectious disease, the term “treating” includes any or all of: preventing the growth, multiplication or replication of the pathogen that causes the infectious disease and ameliorating one or more symptoms of an infectious disease.

Examples of a “mammal” or “animal” include, but are not limited to, a human, rat, mouse, guinea pig, monkey, pig, goat, cow, horse, dog, cat, bird and fowl.

The term “compound”, “cytotoxic agent”, “cytotoxic compound,” “cytotoxic dimer” and “cytotoxic dimer compound” are used interchangeably. They are intended to include compounds for which a structure or formula or any derivative thereof has been disclosed in the present invention or a structure or formula or any derivative thereof that has been incorporated by reference. The term also includes, stereoisomers, geometric isomers, tautomers, solvates, metabolites, salts (e.g., pharmaceutically acceptable salts) and prodrugs, and prodrug salts of a compound of all the formulae disclosed in the present invention. The term also includes any solvates, hydrates, and polymorphs of any of the foregoing. The specific recitation of “stereoisomers,” “geometric isomers,” “tautomers,” “solvates,” “metabolites,” “salt” “prodrug,” “prodrug salt,” “conjugates,” “conjugates salt,” “solvate,” “hydrate,” or “polymorph” in certain aspects of the invention described in this application shall not be interpreted as an intended omission of these forms in other aspects of the invention where the term “compound” is used without recitation of these other forms.

The term “imine reactive reagent” refers to a reagent that is capable of reacting with an imine group. Examples of imine reactive reagent includes, but is not limited to, sulfites (H₂SO₃, H₂SO₂ or a salt of HSO₃⁻, SO₃²⁻ or HSO₂⁻ formed with a cation), metabisulfite (H₂S₂O₅ or a salt of S₂O₅²⁻ formed with a cation), mono, di, tri, and tetra-thiophosphates (PO₃SH₃, PO₂S₂H₃, POS₃H₃, PS₄H₃ or a salt of PO₃S³⁻, PO₂S₂³⁻, POS₃³⁺ or PS₄³⁻ formed with a cation), thio phosphate esters ((R₅O)₂PS(OR₅), R₅SH, R₅SOH, R₅SO₂H, R₅SO₃H), various amines (hydroxyl amine (NH₂OH), hydrazine (NH₂NH₂), NH₂OR₅, R₅NHR_{5′}, NH₂R₅), NH₂—CO—NH₂, NH2-C(═S)—NH₂), thiosulfate (H₂S₂O₃ or a salt of S₂O₃²⁻ formed with a cation), dithionite (H₂S₂O₄ or a salt of S₂O₄²⁻ formed with a cation), phosphorodithioate (P(═S)(OR₅)(SH)(OH) or a salt thereof formed with a cation), hydroxamic acid (RsC(═O)NHOH or a salt formed with a cation), hydrazide (R₅CONHNH₂), formaldehyde sulfoxylate (HOCH₂SO₂H or a salt of HOCH₂SO₂⁻ formed with a cation, such as HOCH₂SO₂⁻Na⁺), glycated nucleotide (such as GDP-mannose), fludarabine or a mixture thereof, wherein R₅ and R_{5′} are each independently a linear or branched alkyl having 1 to 8 carbon atoms and are substituted with at least one substituent selected from —N(R₅)(R_{5′}), —CO₂H, —SO₃H, and —PO₃H; R₅ and R_{5′} can be further optionally substituted with a substituent for an alkyl described herein; Preferably, the cation is a monovalent cation, such as Na⁺ or K⁺. Preferably, the imine reactive reagent is selected from sulfites, hydroxyl amine, urea and hydrazine. More preferably, the imine reactive reagent is NaHSO₃ or KHSO₃.

“Cell binding agents” or “Cell binding molecules” may be of any kind presently known, or that become known, and include peptides and non-peptides. Generally, these can be antibodies (especially monoclonal antibodies) or a fragment of an antibody that contains at least one binding site, lymphokines, hormones, growth factors, nutrient-transport molecules (such as transferrin), or any other cell binding molecule or substance (such as vitamins).

More specific examples of cell binding agents that can be used include: monoclonal antibodies; single chain antibodies; fragments of antibodies such as Fab, Fab′, F(ab′)₂, Fᵥ, {Parham, 131 J. Immunol. 2895-2902 (1983); Spring et al, 113 J. Immunol. 470-478 (1974); Nisonoff et al, 89 Arch. Biochem. Biophys. 230-244 (1960)}, fragments produced by a Fab expression library, anti-idiotypic (anti-Id) antibodies, CDR's, and epitope-binding fragments of any of the above which immunospecifically bind to cancer cell antigens, viral antigens or microbial antigens; interferons; peptides; lymphokines such as IL-2, IL-3, IL-4, IL-6; hormones such as insulin, TRH (thyrotropin releasing hormones), MSH (melanocyte-stimulating hormone), steroid hormones, such as androgens and estrogens; growth factors and colony-stimulating factors such as EGF, TGFα, insulin like growth factor (IGF-I, IGF-II) G-CSF, M-CSF and GM-CSF {Burgess, 5 Immunology Today 155-158 (1984)}; vitamins, such as folate and; transferrin {O'Keefe et al, 260 J. Biol. Chem. 932-937 (1985)}.

Monoclonal antibody technology permits the production of extremely selective cell binding agents in the form of specific monoclonal antibodies. Particularly well known in the art are techniques for creating monoclonal antibodies produced by immunizing mice, rats, hamsters or any other mammal with the antigen of interest such as the intact target cell, antigens isolated from the target cell, whole virus, attenuated whole virus, and viral proteins such as viral coat proteins. Selection of the appropriate cell binding agent is a matter of choice that depends upon the particular cell population that is to be targeted, but in general monoclonal antibodies are preferred if an appropriate one is available.

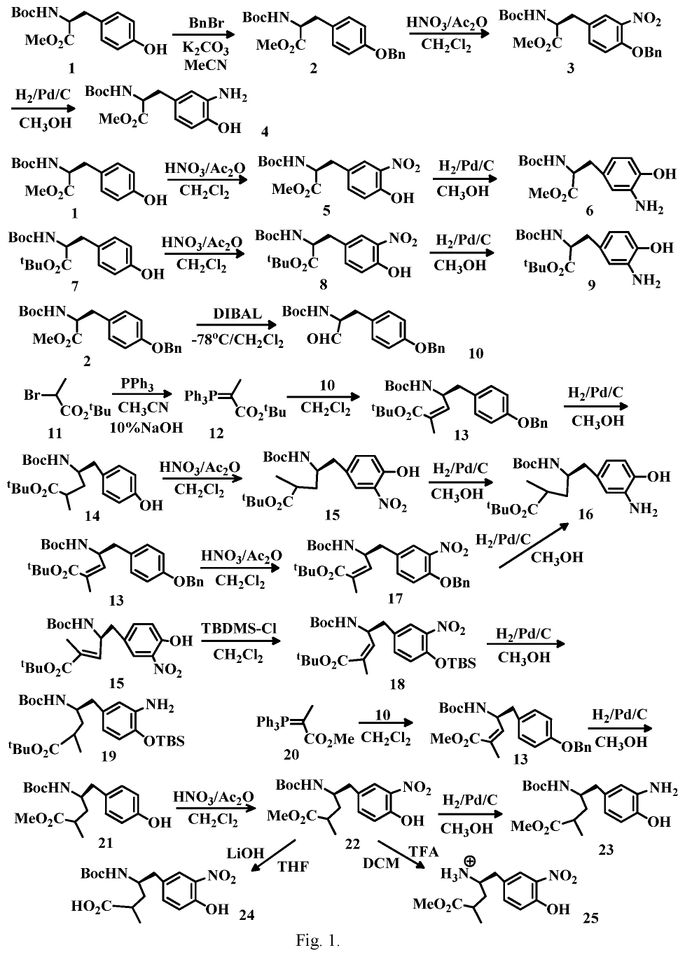

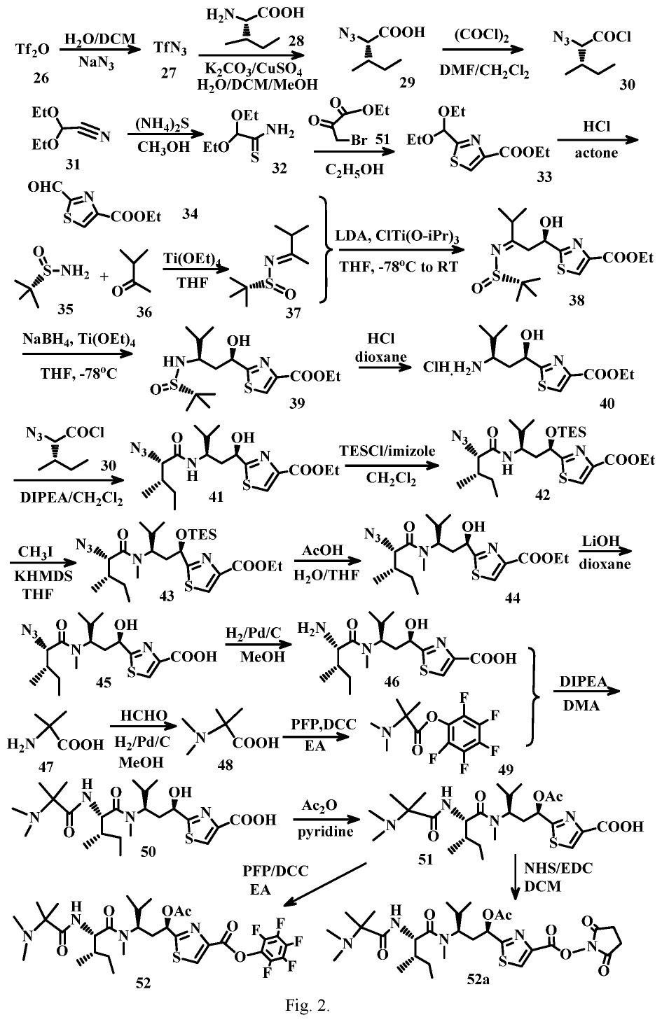

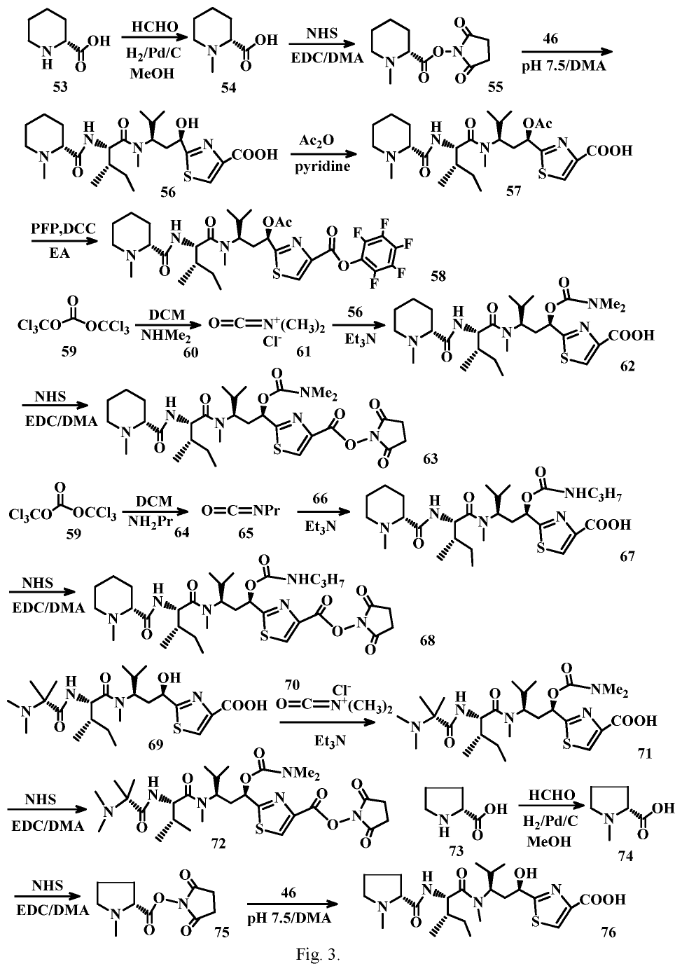

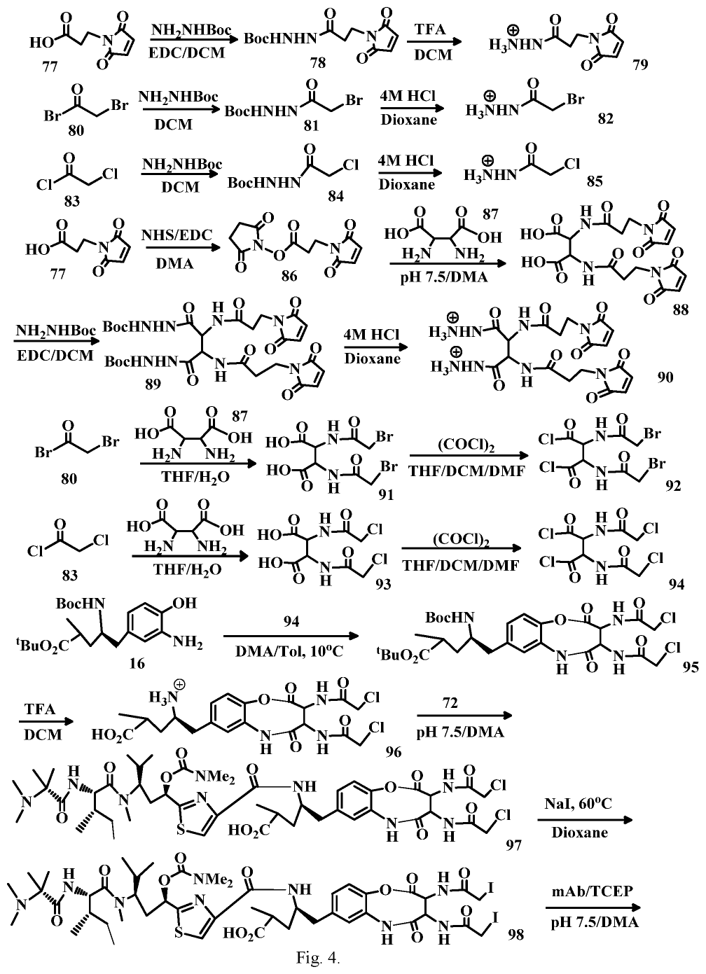

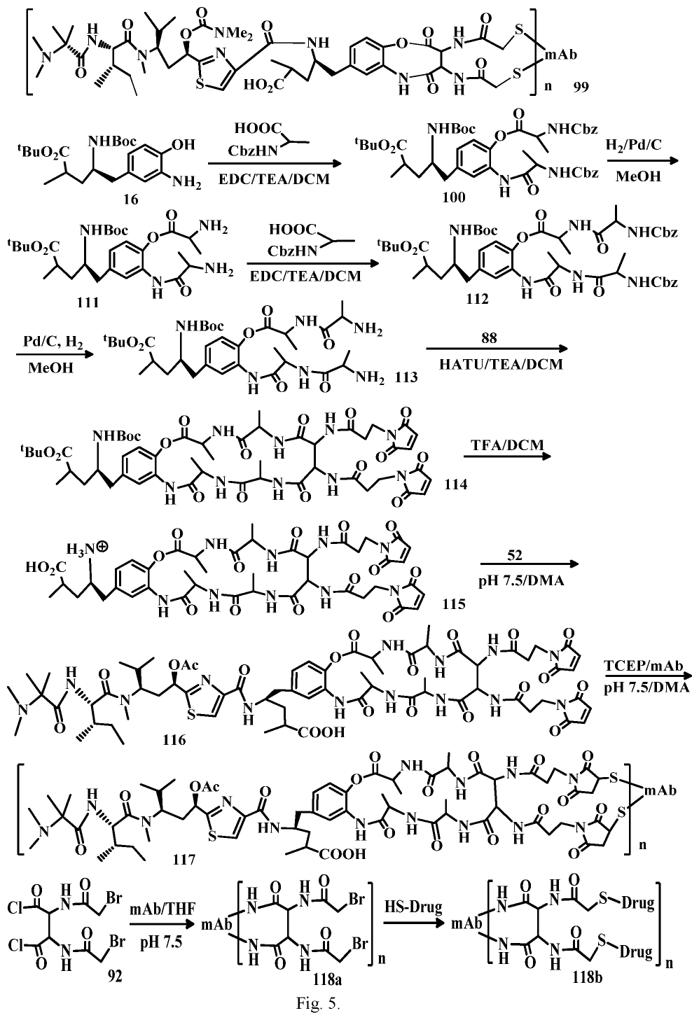

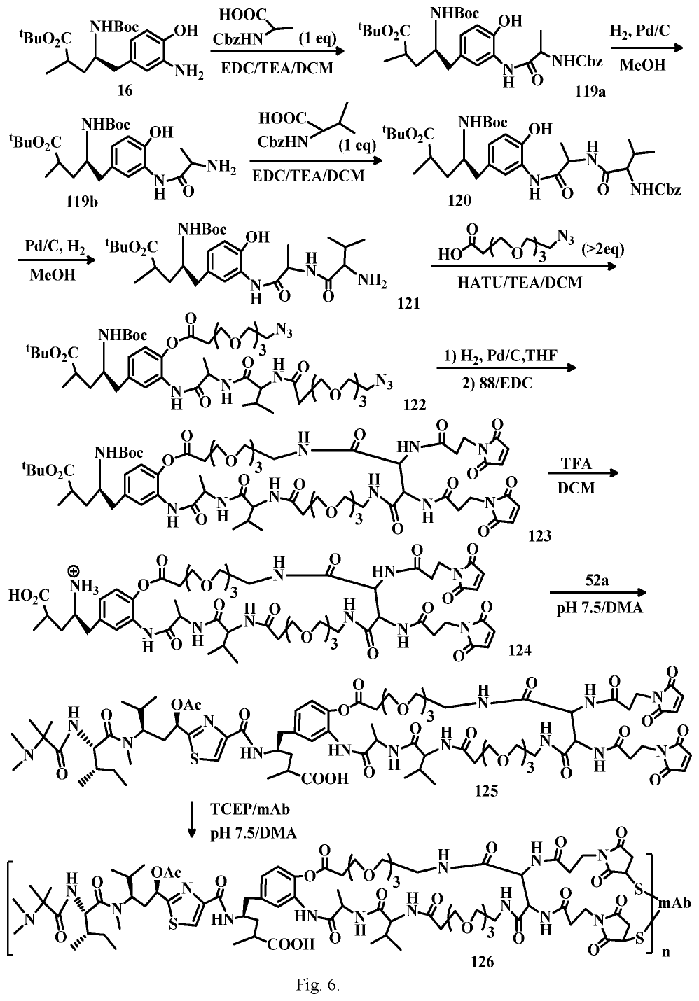

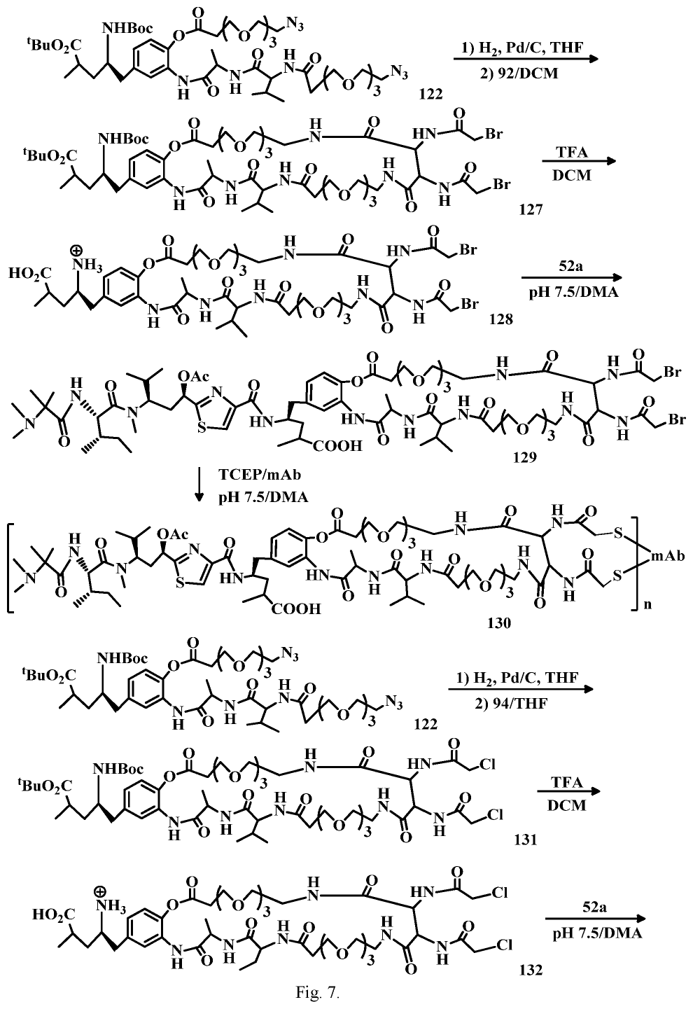

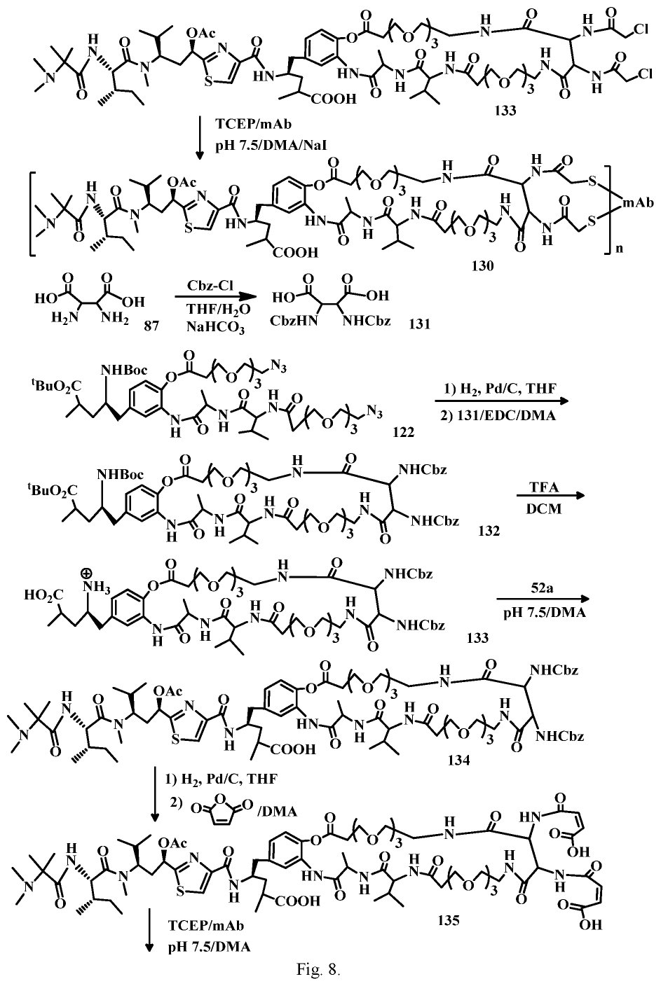

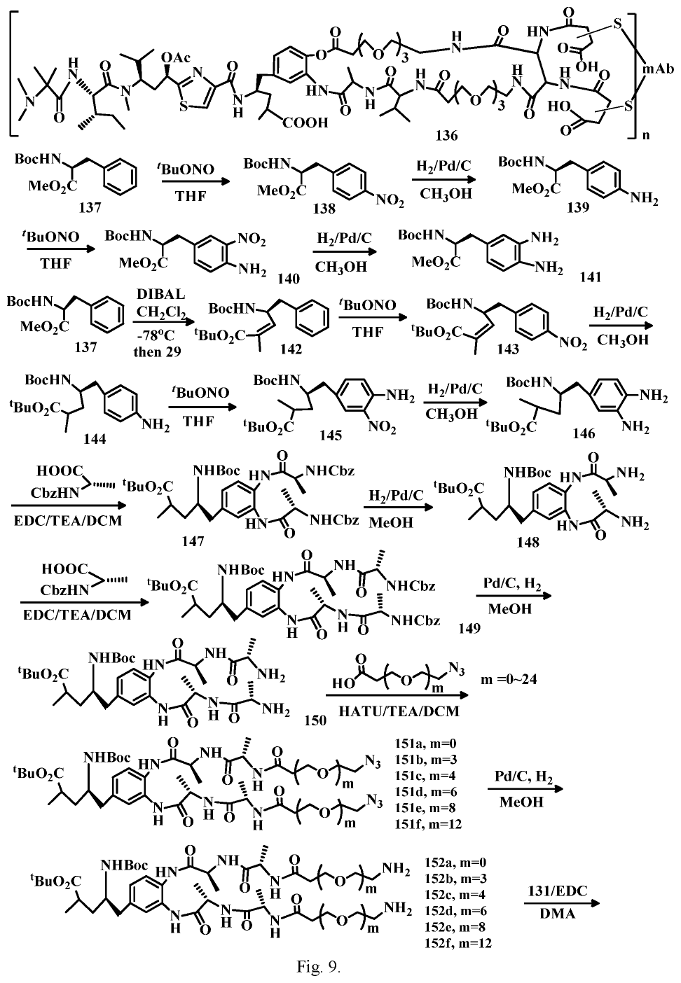

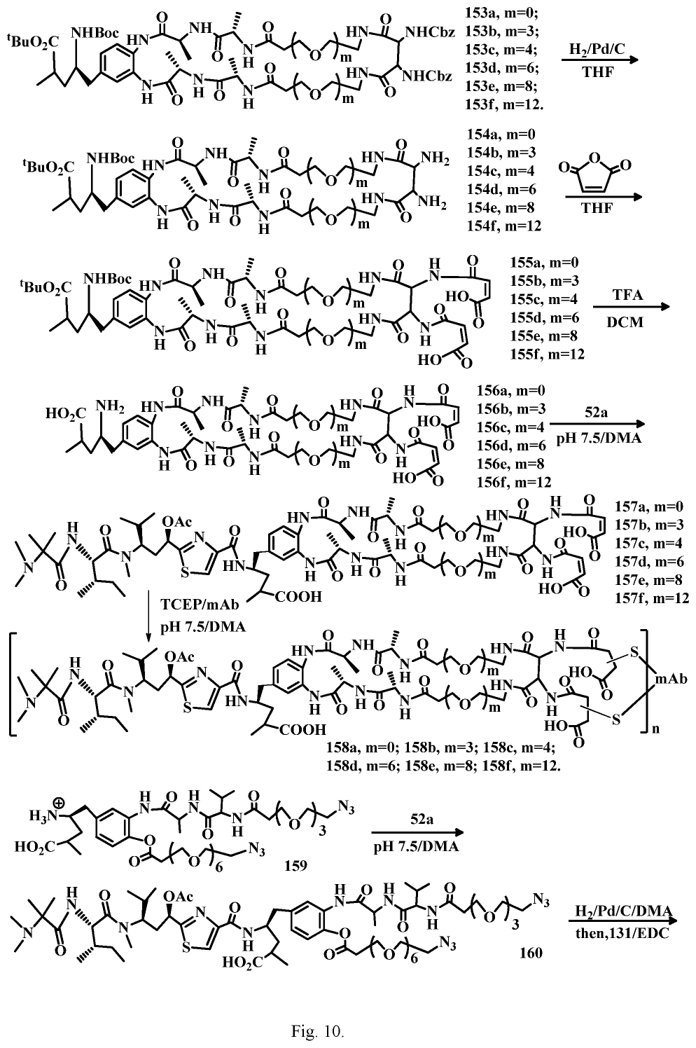

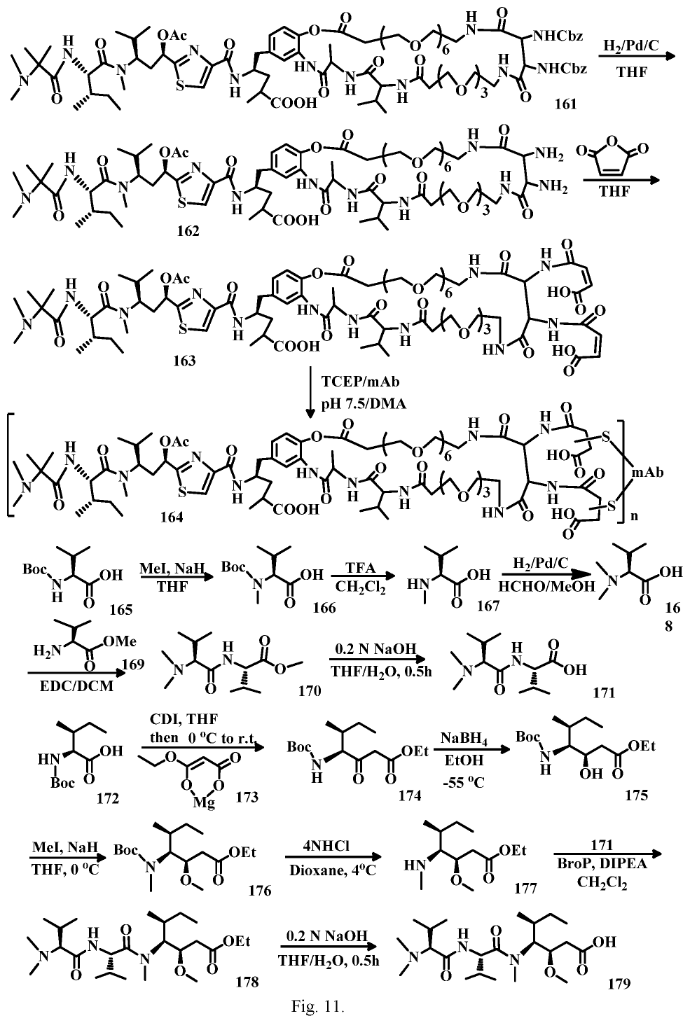

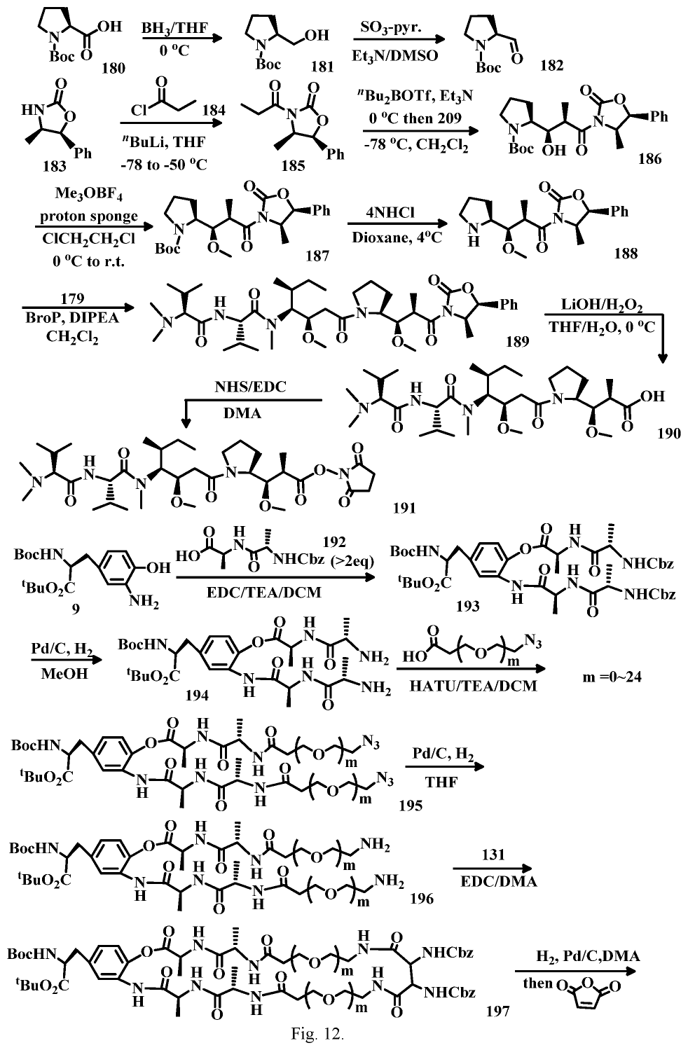

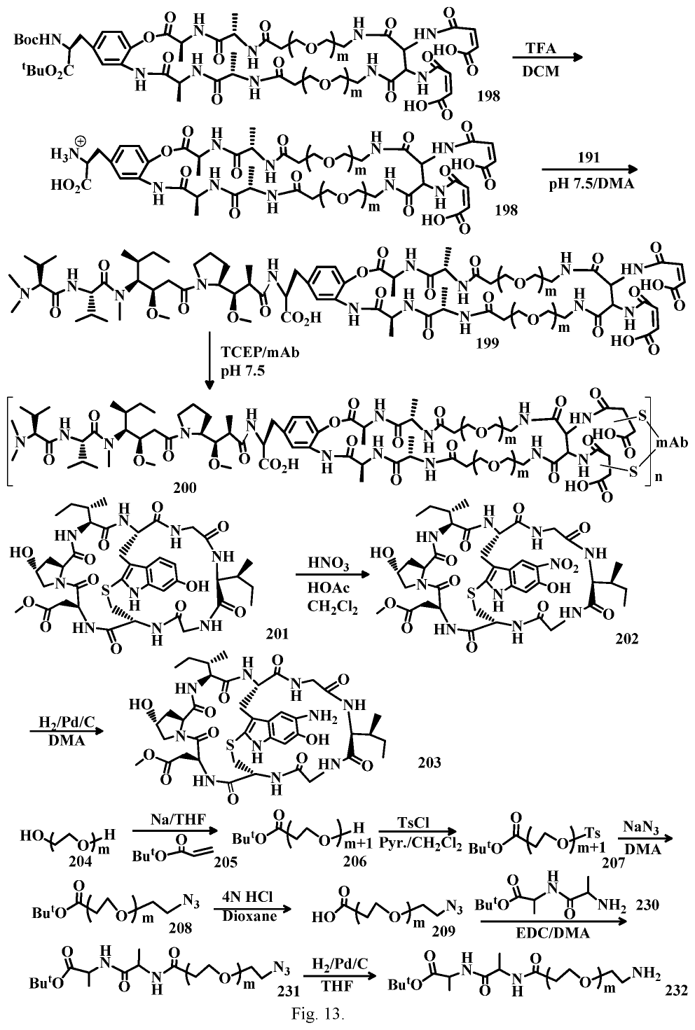

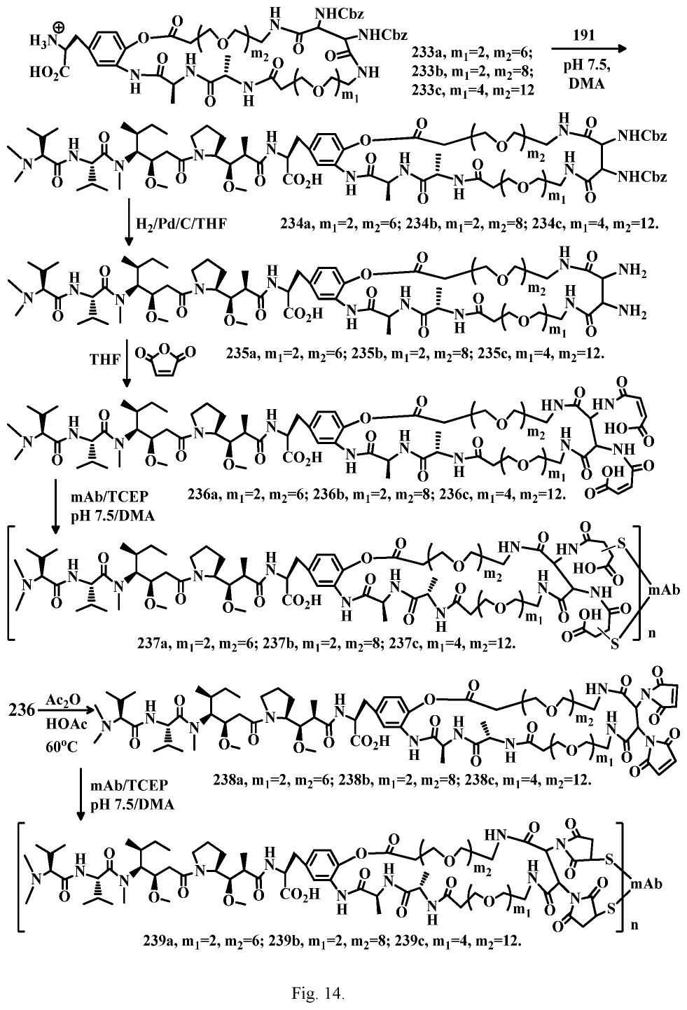

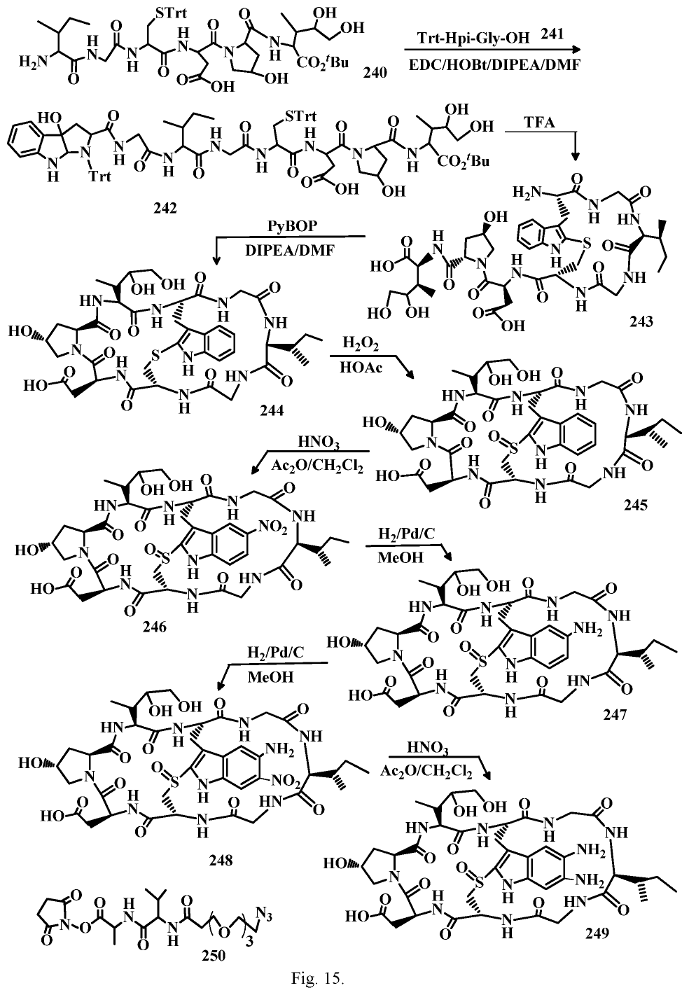

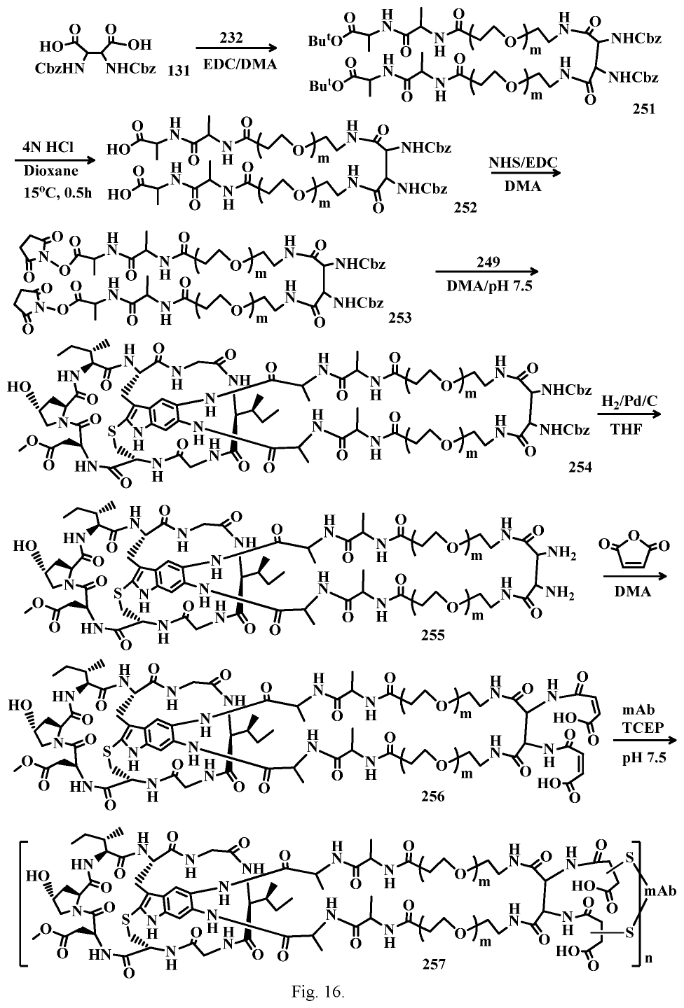

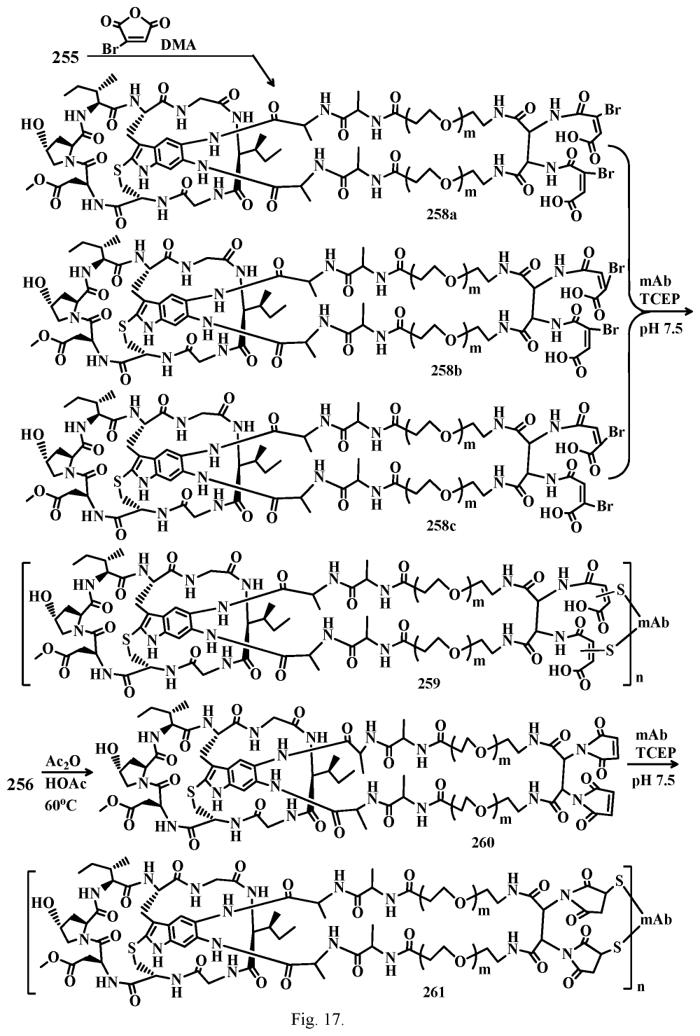

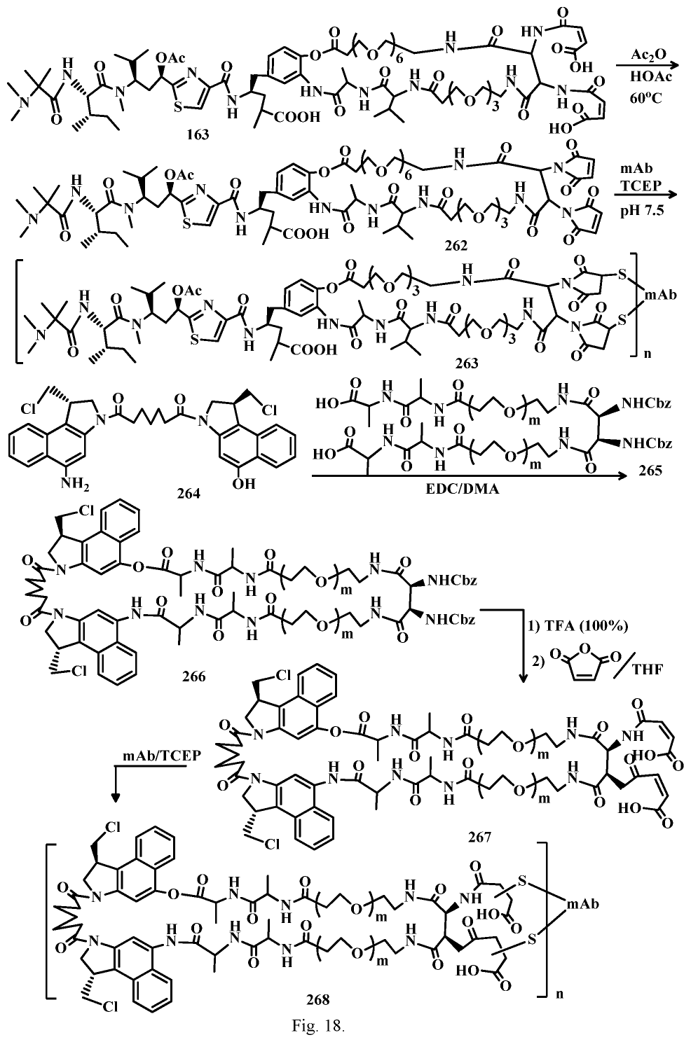

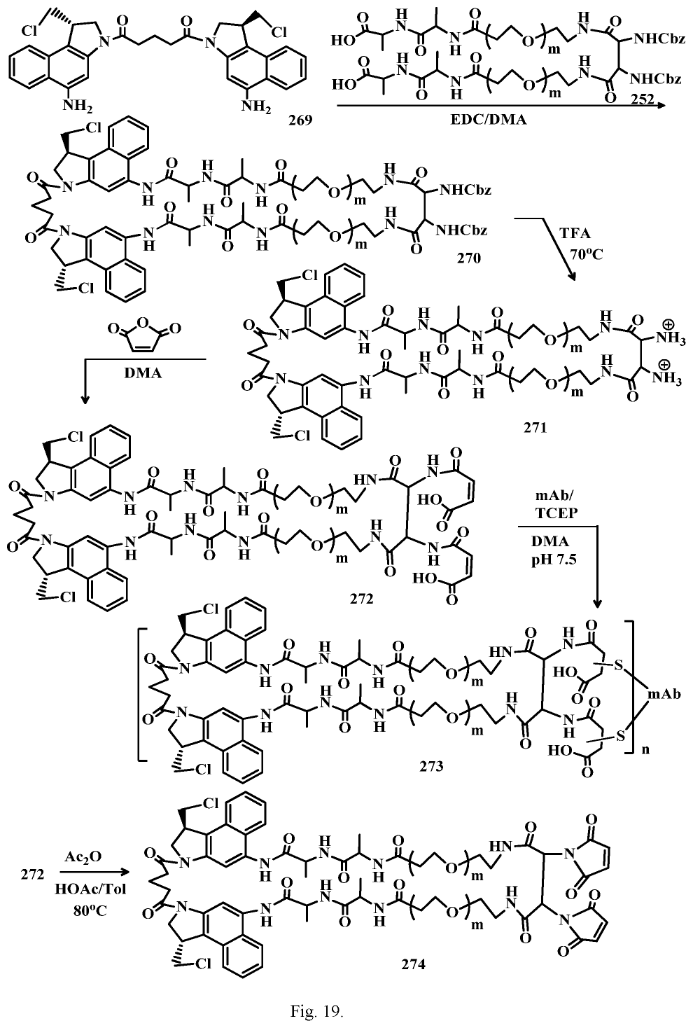

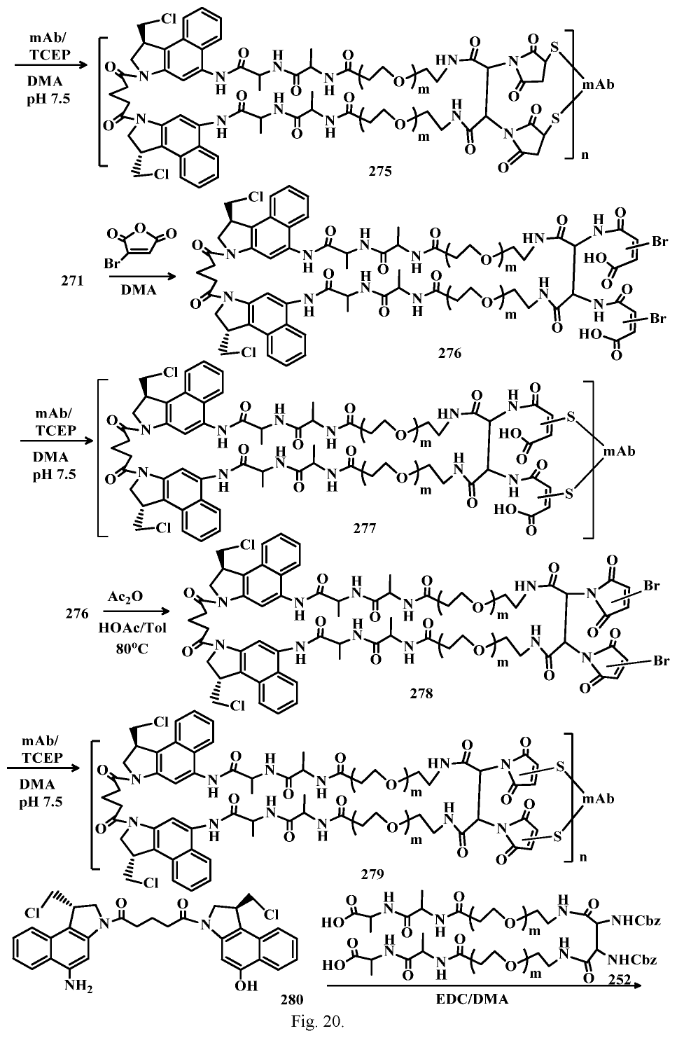

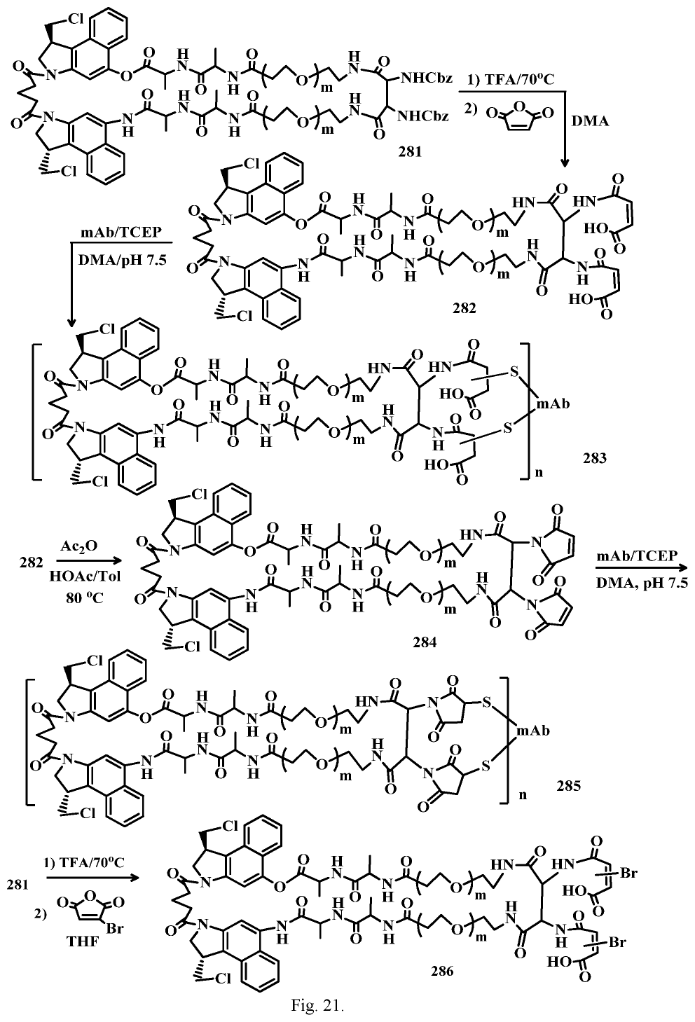

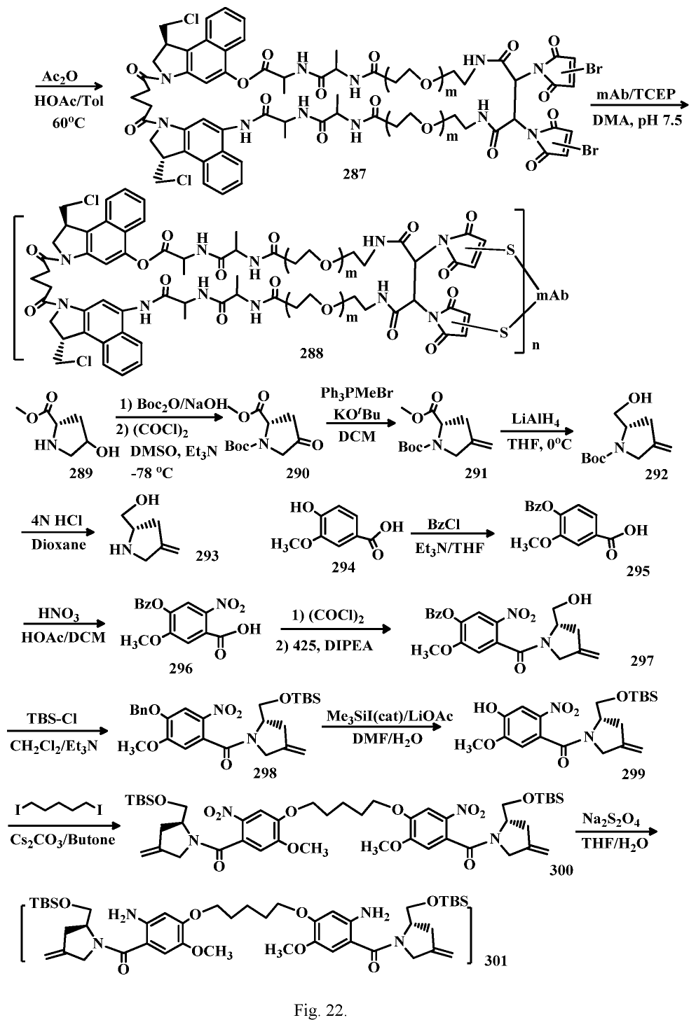

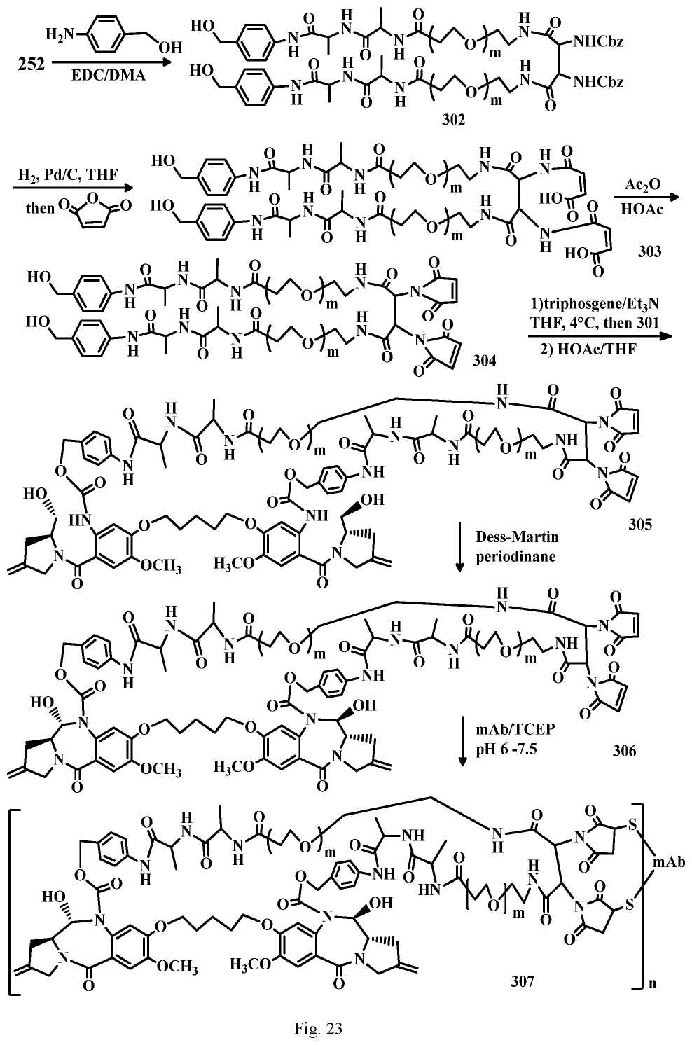

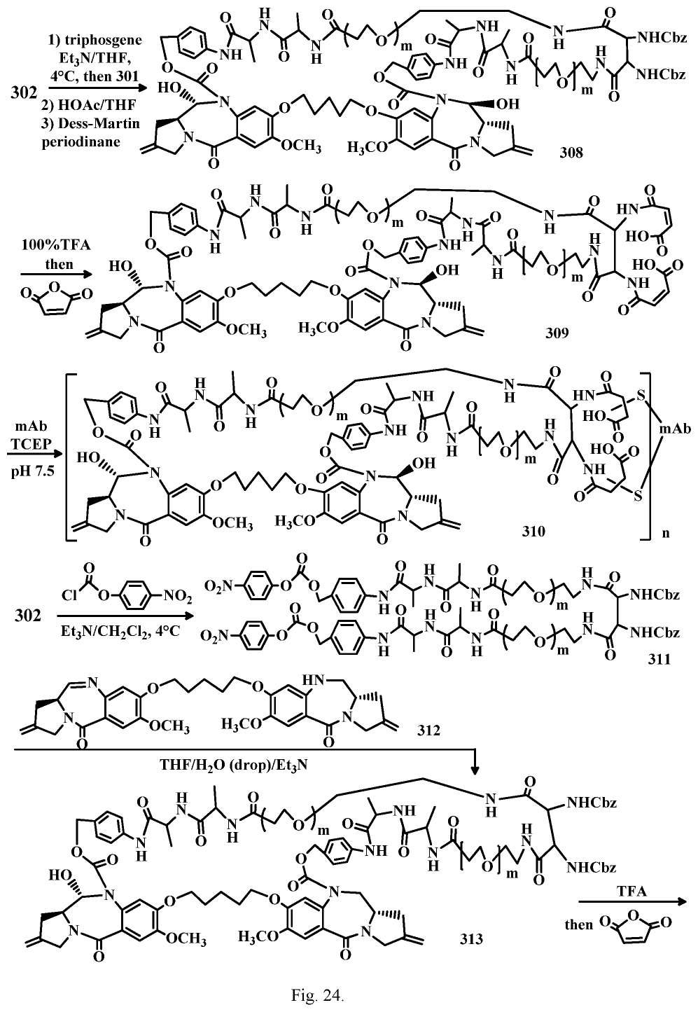

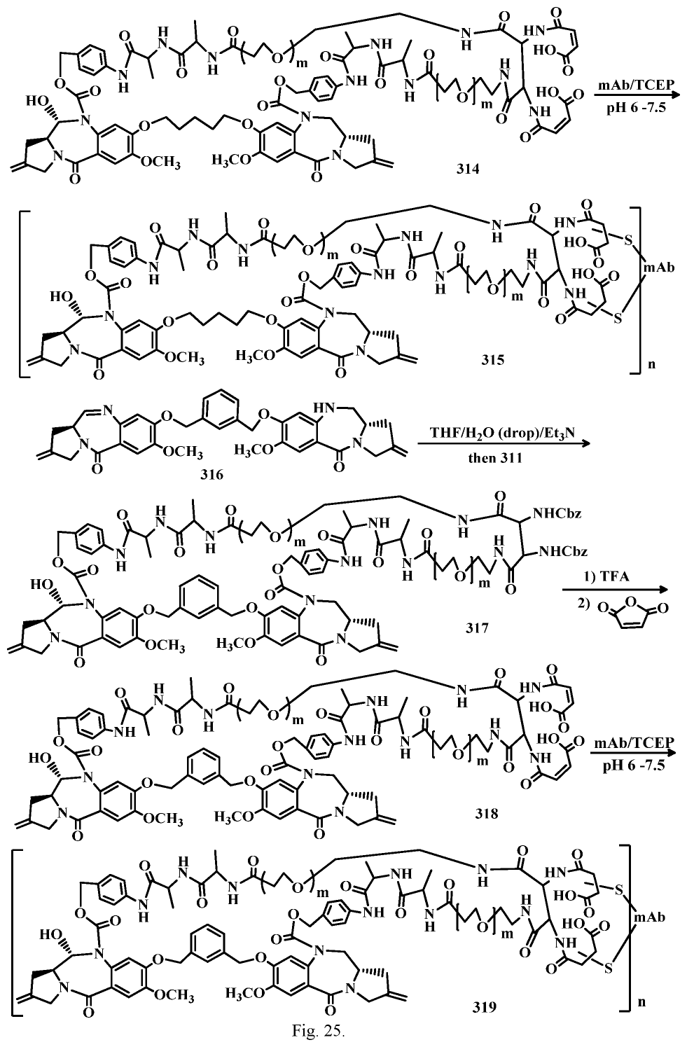

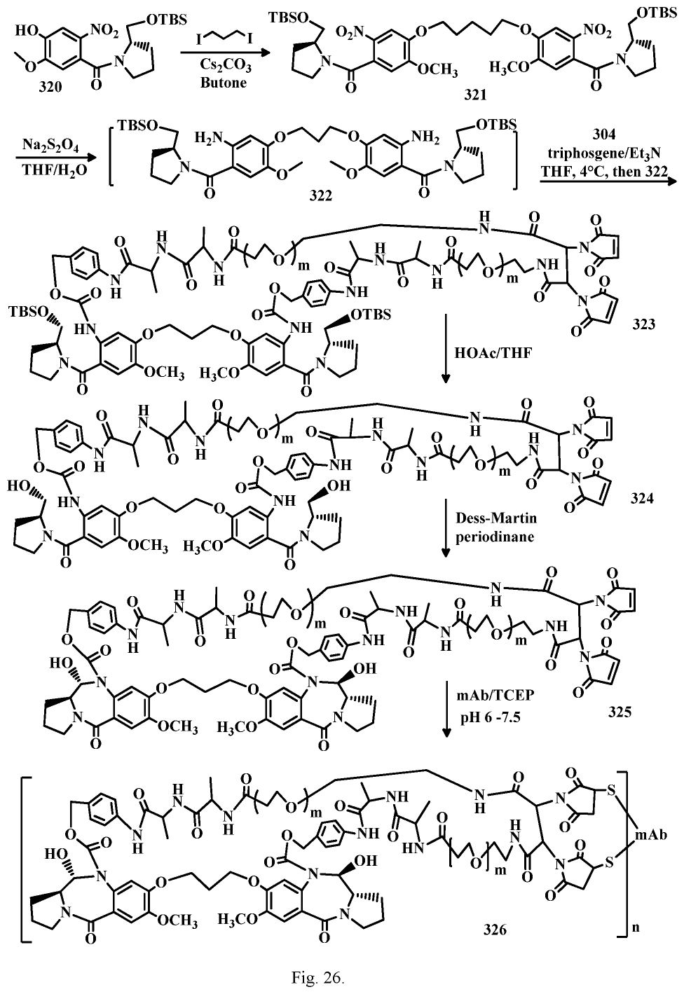

The novel conjugates disclosed herein use the bis-linkers. Examples of some suitable linkers and their synthesis are shown in FIGS. 1 to 26 and in the experimental examples.

A Conjugate of a Cell-Binding Agent-A Cytotoxic Molecule Via a Bis-Linkage Containing 2,3-Diaminosuccinyl Group.

The bis-linkage of the conjugate is represented by Formula (I), (II), (III) or (IV):

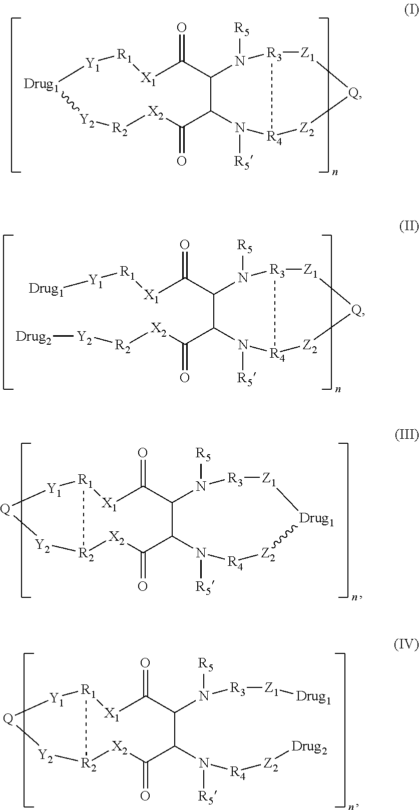

or their optical isomers, racemates, diastereomers or enantiomers;

wherein

“ [symbol] ” represents a single bond;

“ [symbol] ” is optionally either a single bond, or absent;

“-----” is optionally either a single bond, or a double bond, or can optionally be absent;

n is 1 to 30 independently;

Q is a cell-binding agent/molecule that links to R₃ and R₄ can be any kind presently known, or that become known, of a molecule that binds to, complexes with, or reacts with a moiety of a cell population sought to be therapeutically or otherwise biologically modified. Preferably the cell-binding agent/molecule is an immunotherapeutic protein, an antibody, a single chain antibody; an antibody fragment that binds to the target cell; a monoclonal antibody; a single chain monoclonal antibody; or a monoclonal antibody fragment that binds the target cell; a chimeric antibody; a chimeric antibody fragment that binds to the target cell; a domain antibody; a domain antibody fragment that binds to the target cell; adnectins that mimic antibodies; DARPins; a lymphokine; a hormone; a vitamin; a growth factor; a colony stimulating factor; or a nutrient-transport molecule (a transferrin); a binding peptides having over four aminoacids, or protein, or antibody, or small cell-binding molecule or ligand attached on albumin, polymers, dendrimers, liposomes, nanoparticles, vesicles, or (viral) capsids;

Drug₁ or/and Drug₂ are a cytotoxic molecule/agent that is a therapeutic drug/molecule/agent, or an immunotherapeutic protein/molecule, or a function molecule for enhancement of binding or stabilization of the cell-binding agent, or a cell-surface receptor binding ligand, or for inhibition of cell proliferation, or for monitoring, detection or study of a cell-binding molecule action. It can also be an analog, or prodrug, or a pharmaceutically acceptable salt, hydrate, or hydrated salt, or a crystalline structure, or an optical isomer, racemate, diastereomer or enantiomer, of immunotherapeutic compound, a chemotherapeutic compound, an antibody (probody) or an antibody (probody) fragment, or siRNA or DNA molecule, or a cell surface binding ligand;

Preferably a cytotoxic molecule is any of many small molecule drugs, including, but not limited to, tubulysins, calicheamicins, auristatins, maytansinoids, CC-1065 analogs, morpholinos doxorubicins, taxanes, cryptophycins, amatoxins (e.g. amanitins), epothilones, eribulin, geldanamycins, camptothecins (e.g. SN-38), duocarmycins, daunomycins, methotrexates, vindesines, vincristines, and benzodiazepine dimers (e.g., dimers of pyrrolobenzodiazepine (PBD), tomaymycin, indolinobenzodiazepines, imidazobenzothiadiazepines, or oxazolidinobenzodiazepines);

X₁ and X₂ are the same or different, and independently selected from NH; NHNH; N(R₁); N(R₁)N(R₂); O; S; S—S, O—NH. O—N(R₁), CH₂—NH. CH₂—N(R₁), CH═NH. CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NH)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); or C₁-C₆ alkyl; C₂-C₅ alkenyl, heteroalkyl, alkylcycloalkyl, or heterocycloalkyl; C₃-C₅ aryl, Ar-alkyl, heterocyclic, carbocyclic, cycloalkyl, heteroalkylcycloalkyl, alkylcarbonyl, or heteroaryl;

Y₁, Y₂, Z₁ and Z₂ are, the same or different, and independently a function group that link to a cell-binding molecule Q, or drug₁ or drug₂, to form a disulfide, ether, ester, thioether, thioester, peptide, hydrazone, carbamate, carbonate, amine (secondary, tertiary, or quarter), imine, cycloheteroalkyane, heteroaromatic, alkyloxime or amide bond; Preferably Y₁, Y₂, Z₁ and Z₂ independently have the following structures: C(O)CH, C(O)C, C(O)CH₂, ArCH₂, C(O), NH, NHNH, N(R₁), N(R₁)N(R₂), O, S, S—S, O—NH, O—N(R₁), CH₂—NH. CH₂—N(R₁), CH═NH. CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); or C₁-C₈ alkyl, C₂-C₅ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl;

Preferably Y₁, Y₂, Z₁ and Z₂ are linked to pairs of thiols of a cell-binding agent/molecule. The thiols are preferably pairs of sulfur atoms reduced from the inter chain disulfide bonds of the cell-binding agent by a reduction agent selected from dithiothreitol (DTT), dithioerythritol (DTE), L-glutathione (GSH), tris (2-carboxyethyl) phosphine (TCEP), 2-mercaptoethylamine (β-MEA), or/and beta mercaptoethanol (β-ME, 2-ME);

R₁, R₂, R₃, and R₄ are a chain of atoms selected from C, N, O, S, Si, and P, preferably having 0-500 atoms, which covalently connects to X and Z₁, and Y and Z₂. The atoms used in forming R₁, R₂, R₃, and R₄ may be combined in all chemically relevant ways, such as forming alkylene, alkenylene, and alkynylene, ethers, polyoxyalkylene, esters, amines, imines, polyamines, hydrazines, hydrazones, amides, ureas, semicarbazides, carbazides, alkoxyamines, alkoxylamines, urethanes, amino acids, peptides, acyloxylamines, hydroxamic acids, or combination above thereof. Preferably R₁, R₂, R₃, and R₄ are, the same or different, independently selected from O, NH, S, NHNH, N(R₅), N(R₃)N(R_{3′}), polyethyleneoxy unit of formula (OCH₂CH₂)ₚOR₅, or (OCH₂CH—(CH₃))ₚOR₅, or NH(CH₂CH₂O)ₚR₅, or NH(CH₂CH(CH₃)O)ₚR₅, or N[(CH₂CH₂O)ₚR₅]—[(CH₂CH₂O)_{p′}R_{5′}], or (OCH₂CH₂)ₚCOOR₅, or CH₂CH₂(OCH₂CH₂)ₚCOOR₅, wherein p and p′ are independently an integer selected from 0 to about 1000, or combination thereof; C₁-C₈ alkyl; C₂-C₈ heteroalkyl, or alkylcycloalkyl, heterocycloalkyl; C₃-C₈ aryl, Ar-alkyl, heterocyclic, carbocyclic, cycloalkyl, heteroalkylcycloalkyl, alkylcarbonyl, or heteroaryl; wherein R₅ and R_{5′} are independently H; C₁-C₈ alkyl; C₂-C₈ heteroalkyl, alkylcycloalkyl, or heterocycloalkyl; C₃-C₈ aryl, Ar-alkyl, heterocyclic; C₂-C₈ carbon atoms esters, ether, or amide; or 1˜24 amino acids;

More preferably R₁, R₂, R₃, R₄, R₅ and R_{5′} are independently H; C₁-C₈ alkyl; C₂-C₈ heteroalkyl, alkylcycloalkyl, or heterocycloalkyl; C₃-C₈ aryl, Ar-alkyl, heterocyclic, carbocyclic, heteroalkylcycloalkyl, alkylcarbonyl, or heteroaryl; or C₂-C₈ carbon atoms esters, ether, or amide; or 1-24 amino acids; or polyethyleneoxy having formula (OCH₂CH₂)ₚ or (OCH₂CH(CH₃))ₚ, wherein p is an integer from 0 to about 5000, or combination above thereof;

R₁, R₂, R₃, and R₄ may optionally be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1-8 natural or unnatural amino acid unites. The natural aminoacid is preferably selected from aspartic acid, glutamic acid, arginine, histidine, lysine, serine, threonine, asparagine, glutamine, cysteine, selenocysteine, tyrosine, phenylalanine, glycine, proline, tryptophan, and alanine;

Additionally R₁, R₂, R₃, and R₄ may independently contain one of the following hydrophilic structures:

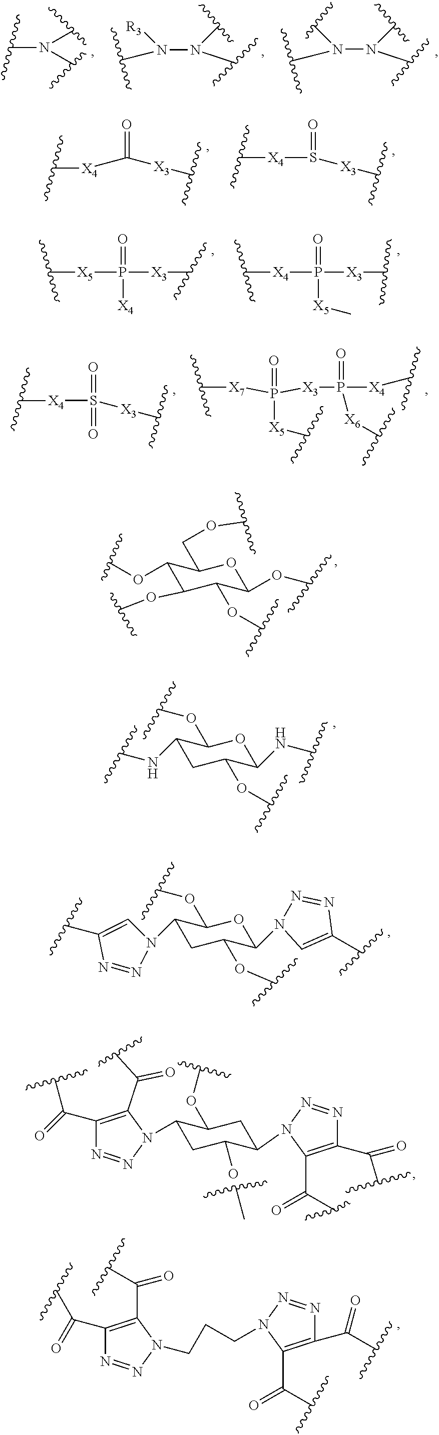

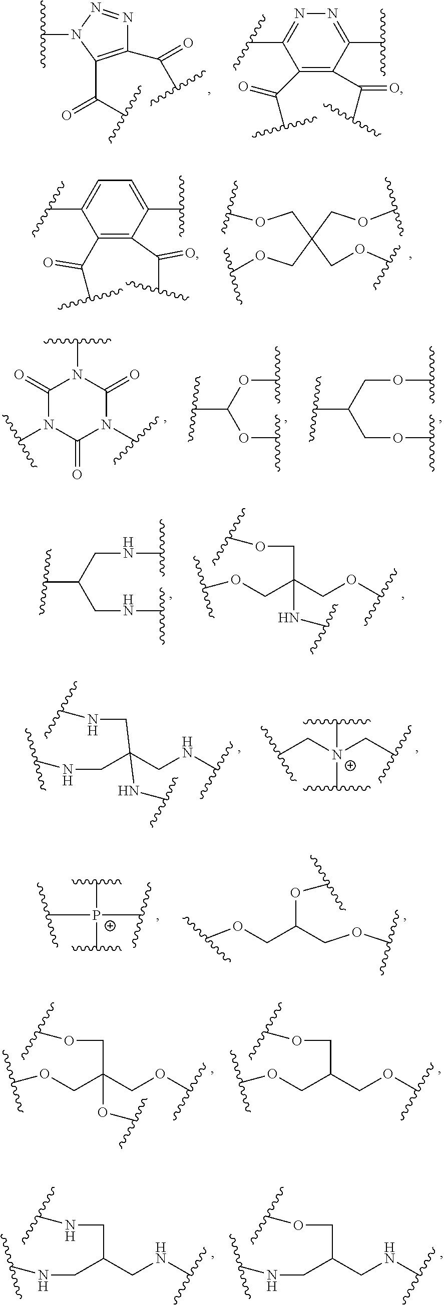

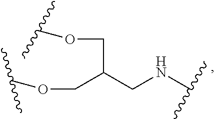

wherein [symbol] is the site of linkage; X₃, X₄, X₅, X₆, and X₇, are independently selected from NH; NHNH; N(R₅); N(R₅)N(R_{5′}); O; S; C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, heteroaryl; or 1-8 amino acids; wherein R₅ and R_{5′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, heteroalkylcycloalkyl, alkylcarbonyl, or heteroaryl; C₁-C₈ esters, ether, or amide; or polyethyleneoxy having formula (OCH₂CH₂)ₚ or (OCH₂CH(CH₃))ₚ, wherein p is an integer from 0 to about 5000, or combination above thereof;

R₁, R₂, R₃, and R₄ Y₁, Y₂, Z₁, and Z₂ may also independently contain a self-immolative or a non-self-immolative component, peptidic units, a hydrazone bond, a disulfide, an ester, an oxime, an amide, or a thioether bond. The self-immolative unit includes, but is not limited to, aromatic compounds that are electronically similar to the para-aminobenzylcarbamoyl (PAB) groups such as 2-aminoimidazol-5-methanol derivatives, heterocyclic PAB analogs, beta-glucuronide, and ortho or para-aminobenzylacetals;

Preferably, the self-immolative linker component has one of the following structures:

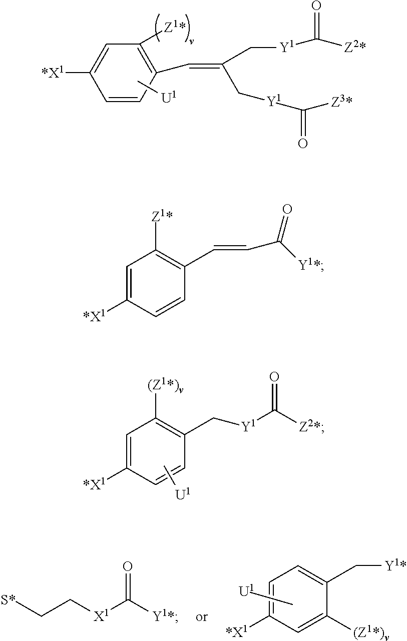

wherein the (*) atom is the point of attachment of additional spacer or releasable linker units, or the cytotoxic agent, and/or the binding molecule (CBA); X¹, Y¹, Z² and Z³ are independently NH, O, or S; Z¹ is independently H, NHR₅, OR₁, SR₅, COX₁R₅, wherein X₁ and R₅ are defined above; v is 0 or 1; U¹ is independently H, OH, C₁-C₆ alkyl, (OCH₂CH₂)ₙ, F, Cl, Br, I, OR₅, SR₅, NR₅R_{5′}, N═NR₅, N═R₅, NR₅R_{5′}, NO₂, SOR₅R_{5′}, SO₂R₅, SO₃R₅, OSO₃R₅, PR₅R_{5′}, POR₅R_{5′}, PO₂R₅R_{5′}, OPO(OR₅)(OR_{5′}), or OCH₂PO(OR₅(OR_{5′}), wherein R₅ and R₅′ are independently selected from H, C₁˜C₈ alkyl; C₂˜C₈ alkenyl, alkynyl, heteroalkyl, or amino acid; C₃˜C₈ aryl, heterocyclic, carbocyclic, cycloalkyl, heterocycloalkyl, heteroaralkyl, alkylcarbonyl, or glycoside; or pharmaceutical cation salts;

The non-self-immolative linker component is one of the following structures:

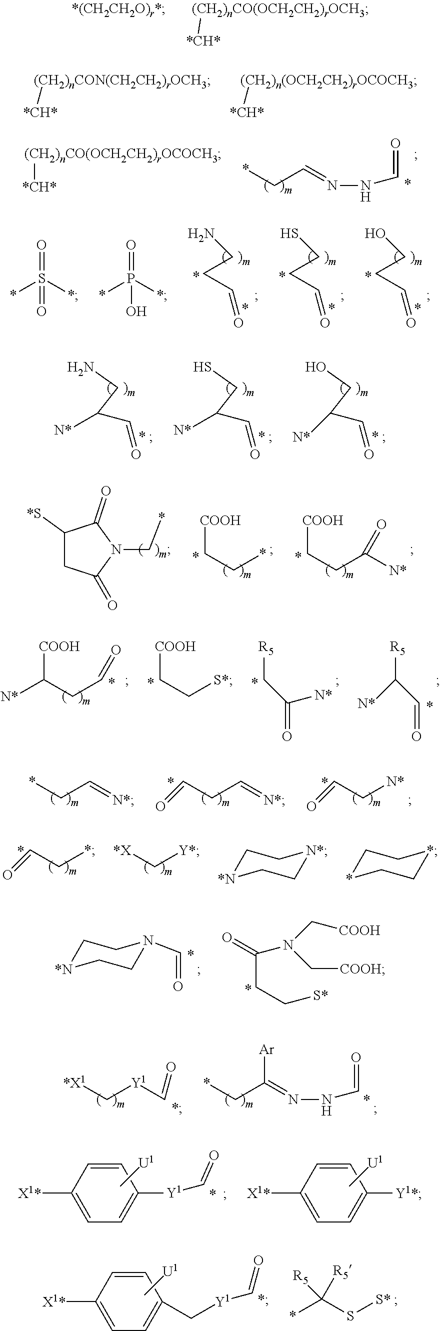

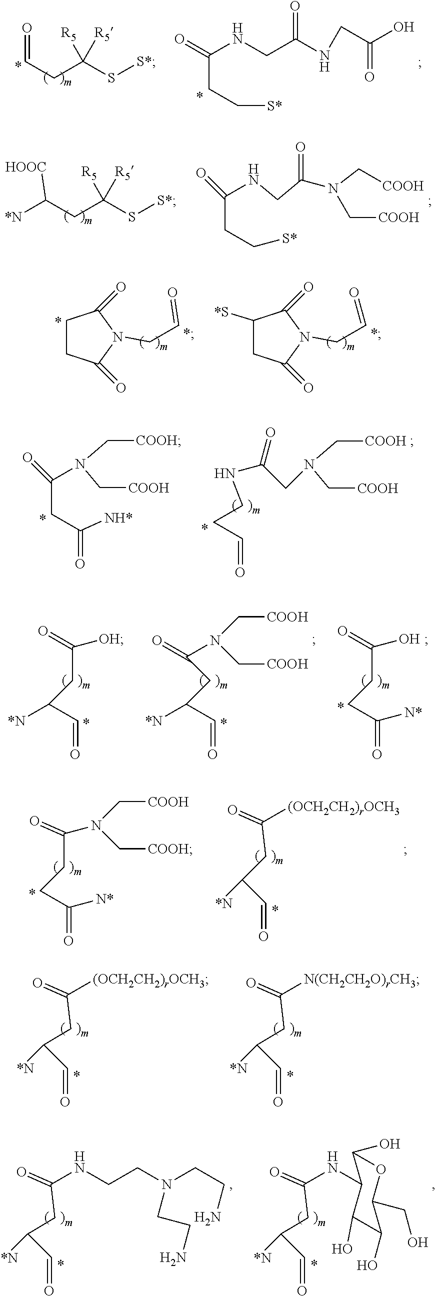

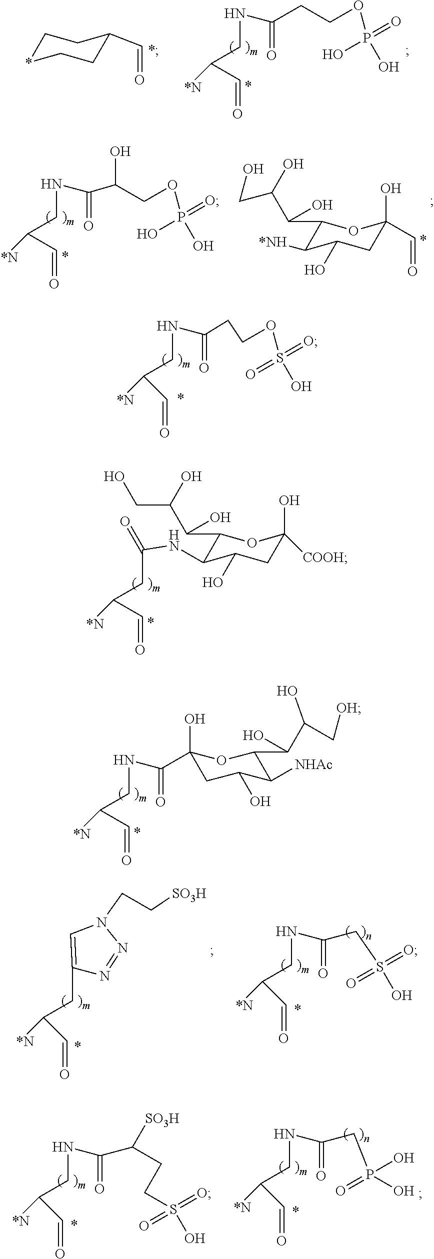

wherein the (*) atom is the point of attachment of additional spacer or releasable linkers, the cytotoxic agents, and/or the binding molecules; X¹, Y¹, U¹, R₅, R_{5′} are defined as above; r is 0-100; m and n are 0-6 independently;

Further preferably, R₁, R₂, R₃, and R₄ may independently contain a releasable linker component. The term releasable linker component includes at least one bond that can be broken under physiological conditions, such as a pH-labile, acid-labile, base-labile, oxidatively labile, metabolically labile, biochemically labile or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with a intracellular thiol, such as a millimolar range of abundant of glutathione inside the malignant cells;

Examples of the releasable linker component R₁, R₂, R₃, and R₄ include, but not limited:

—(CR₅R₆)ₘ(Aa)r(CR₇R₈)ₙ(OCH₂CH₂)ₜ—, —(CR₅R₆)ₘ(CR₇R₈)ₙ(Aa)ᵣ(OCH₂CH₂)ₜ—, -(Aa)ᵣ-(CR₅R₆)ₘ(CR₇R₈)ₙ(OCH₂CH₂)ₜ—, —(CR₅R₆)ₘ(CR₇R₈)ₙ(OCH₂CH₂)ᵣ(Aa)ₜ, —(CR₅R₆)ₘ—(CR₇═CR₈)(CR₉R₁₀)ₙ(Aa)ₜ(OCH₂CH₂)ᵣ—, —(CR₅R₆)ₘ(NR₁₁CO)(Aa)ₜ(CR₉R₁₀)ₙ₋(OCH₂CH₂)ᵣ—, -(CR₅R₆)ₘ(Aa)ₜ(NR₁₁CO)(CR₉R₁₀)ₙ(OCH₂CH₂)ᵣ—, —(CR₅R₆)ₘ(OCO)(Aa)ₜ(CR₉R₁₀)ₙ₋(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ(CR₉R₁₀)ₙ₋(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)ₘ—(OCO)(Aa)ₜ(CR₉R₁₀)ₙ₋(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ(CR₉R₁₀)ₙ₋(OCH₂CH₂)ᵣ—, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CR₅R₆)ₘ-oxazolyl-CO(Aa)ₜ(CR₇R₈)ₙ—, —(CR₅R₆)ₘ-thiazolyl-CO(Aa)ₜ(CCR₇R₈)ₙ—, —(CR₅R₆)ₜ-thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ-(CR₇R₈)ₙ—, —(CR₅R₆)ₜpiperazino-CO(Aa)ₜ-(CR₇R₈)ₙ—, —(CR₅R₆)ₜ—N-methylpiperazin-CO(Aa)ₜ-(CR₇R₈)ₙ—, —(CR₅R)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazolyl(Aa)ₜ-, —(CR₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)r(CR₇R₈)ₙ(OCH₂CH₂)ₜ—, —K(CR₅R₆)ₘ(CR₇R₈)ₙ(Aa)ᵣ(OCH₂CH₂)ₜ—, —K(Aa)ᵣ-(CR₅R₆)ₘ(CR₇R₈)ₙ(OCH₂CH₂)ₜ—, —K(CR₅R₆)ₘ(CR₇R₈)ₙ(OCH₂CH₂)ᵣ(Aa)ₜ-, —K(CR₅R₆)ₘ—(CR₇═CR₈)(CR₉R₁₀)ₙ(Aa)ₜ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ(Aa)ₜ(NR₁₁CO)(CR₉R₁₀)ₙ(OCH₂CH₂)ᵣ—, —K(CR₅R₆)ₘ(OCO)(Aa)ₜ(CR₉R₁₀)ₙ—(OCH₂CH₂)ᵣ—, —K(CR₅R₆)ₘ(OCNR₇)(Aa)ₜ(CR₉R₁₀)ₙ(OCH₂CH₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ(OCNR₇)(Aa)ₜ(CR₉R₁₀)ₙ(OCH₂CH₂)ᵣ—, —K—(CR₅R₆)ₘ(CO)(Aa)ₜ(CR₉R₁₀)ₙ(OCH₂CH₂)ᵣ—, —K(CR₅R₆)ₘ-phenyl-CO(Aa)ₜ(CR₇R₈)ᵣ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ-, —K(CR₅R₆)ₘ-thiazolyl-CO(Aa)ₜ(CR₇R₈)ₙ—, —K(CR₅R₆)ₜ-thienyl-CO(CR₇R₈)ₙ—, —K(CR₅R₆)ₜimidazolyl-CO—(CR₇R₈)ₙ—, —K(CR₅R₆)ₜmorpholino-CO(Aa)ₜ(CR₇R₈)ₙ—, —K(CR₅R₆)ₜpiperazino-CO(Aa)ₜ-(CR₇R₈)ₙ—, —K(CR₅R₆)ₜ—N-methylpiperazinCO(Aa)ₜ(CR₇R₈)ₙ—, —K(CR₆R)ₘ(Aa)ₜphenyl, —K—(CR₅R₆)ₘ-(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘN-methylpiperazino(Aa)ₜ-; wherein m, Aa, m, and n are described above; t and r are 0-100 independently; R₃, R₄, R₅, R₆, R₇, and R₈ are independently chosen from 1-; halide; C₁˜C₈ alkyl; C₂˜C₈ aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)N—H—NH—, O, S, Se, B, Het (heterocyclic or heteroaromatic ring having C₃-C₈), or peptides containing 1-20 amino acids;

More preferably, R₁, R₂, R₃, and R₄, are independently linear alkyl having from 1-18 carbon atoms, or polyethyleneoxy unit having formula (OCH₂CH₂)ₚ, p=1˜5000, or a peptide containing 1˜20 units of aminoacids (L or D form), or combination above.

In addition, Y₁, Y₂, R₁, R₂, R₃, R₄, Z₁ or Z₂ may independently be composed of one or more following components as shown below:

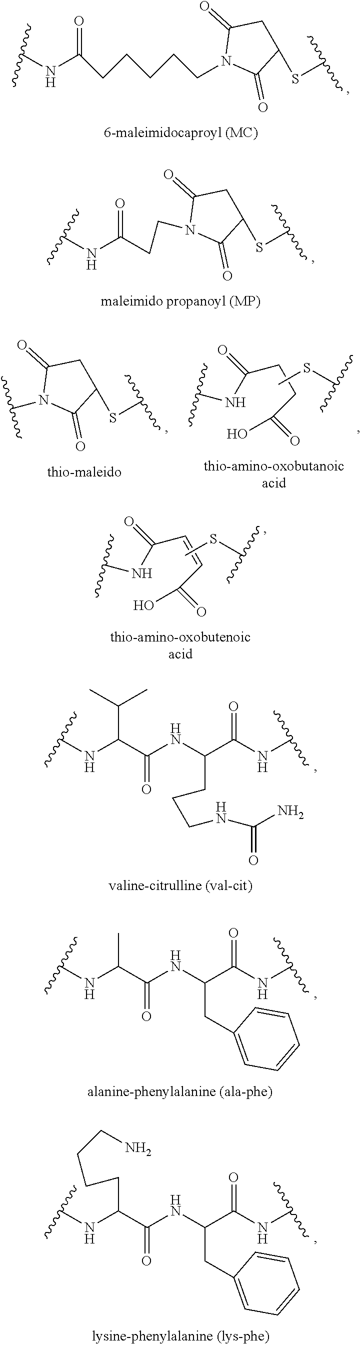

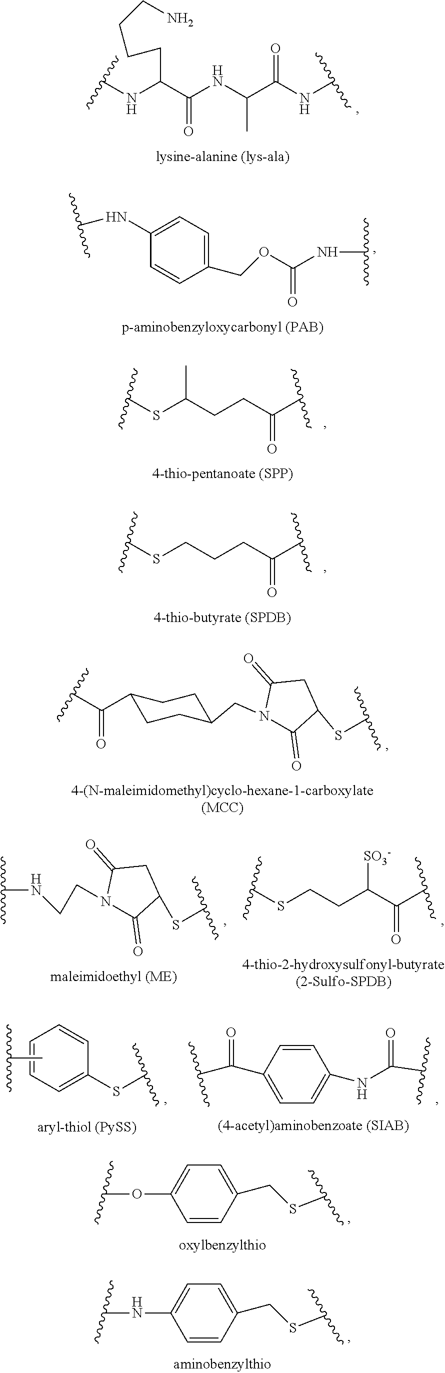

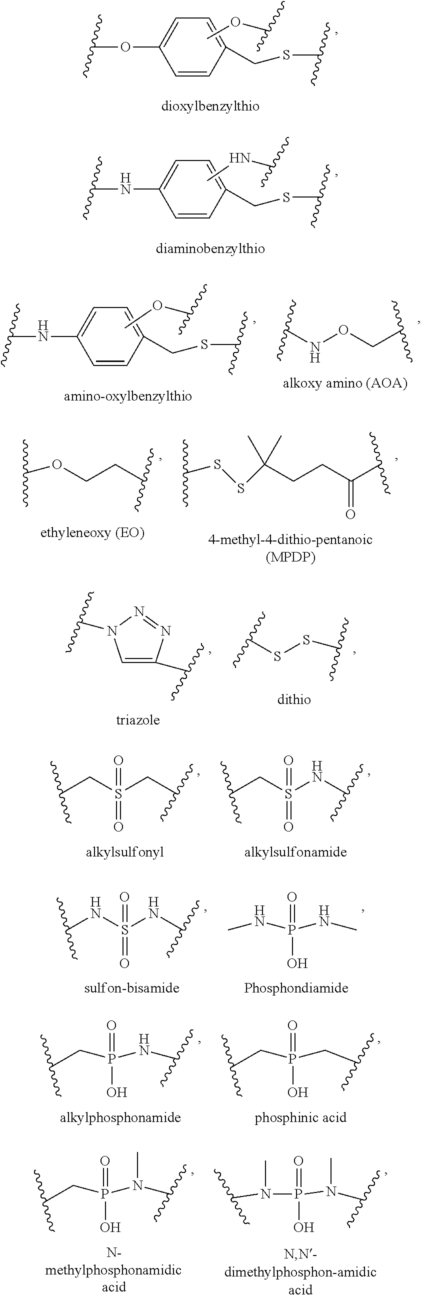

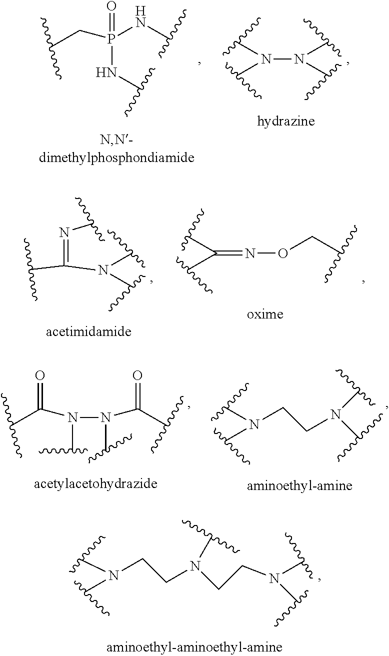

and L- or D-, natural or unnatural peptides containing 1-20 amino acids; wherein a connecting bond in the middle of atoms means that it can connect either neighbor carbon atom bonds; wavery line is the site wherein another bond can be connected to;

Alternatively, Y₁, Y₂, R₁, R₂, R₃, R₄, Z₁ or Z₂, can be independently absent, but Y₁, Y₂, R₁, R₂, R₃, R₄, Z₁ and Z₂ may not be absent at the same time.

A preferred stereoisomer of the Formula (I), (II), (III) and (IV) are presented by the following Formula (Ia), (Ib), (Ic), (IIa), (IIb), (IIc), (IIIa), (IIIb), (IIIc), (IVa), (IVb) and (IVc):

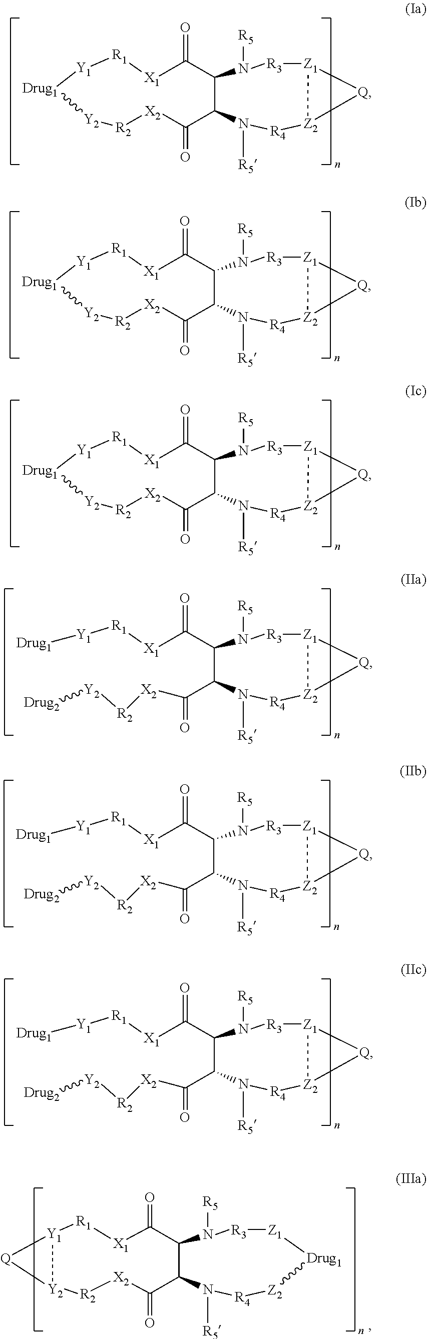

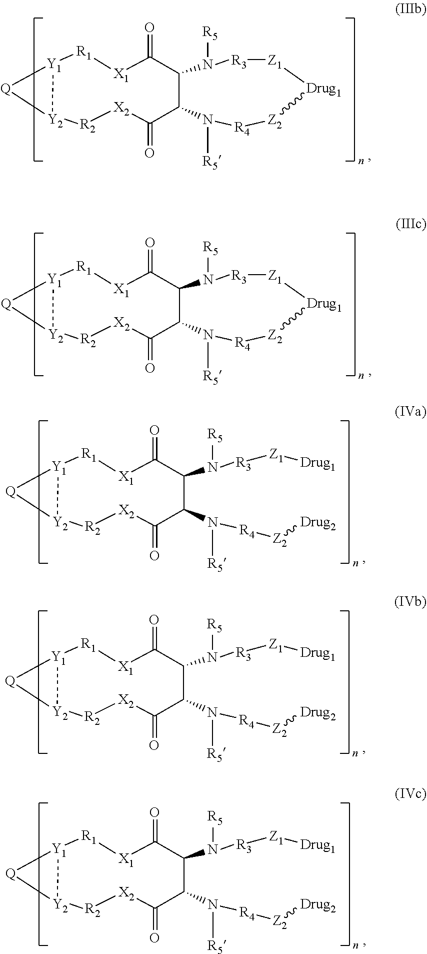

wherein “-----”, Q, X₁, X₂, Y₁, Y₂, R₁, R₂, R₃, R₄, R₅, R_{5′}, Z₁, Z₂, Drug₁ and Drug₂ are defined the same above.

Preferably bis-linkage of the conjugate is further represented by Formula (I-01), (I-02), (I-03), (I-04), (I-05), (I-06), (I-07), (I-08), (I-09), (I-10), (I-11), (I-12), (I-13), (I-14), (I-15), (I-16), (I-17), (I-18), (I-19), (I-20), (I-21), (I-22), (I-23), (II-01), (II-02), (II-03), (II-04), (II-05), (II-06), (II-07), (II-08), (II-09), (II-10), (II-11), (II-12), (II-13), (II-14), (II-15), (II-16), (II-17), (II-18), (III-01), (III-02), (III-03), (III-04), (III-05), (III-06), (III-07), (III-08), (III-09), (III-10), (III-11), (III-12), (III-13), (III-14), (III-15), (III-16), (III-17), (III-18), (III-19), (III-20), (IV-01), (IV-02), (IV-03), (IV-04), (IV-05), (IV-06), (IV-07), (IV-08), (IV-09), (IV-10), (IV-11), (IV-12), (IV-13), (IV-14), (IV-15), (IV-16), (IV-17), (IV-18), (IV-19), and (IV-20) below:

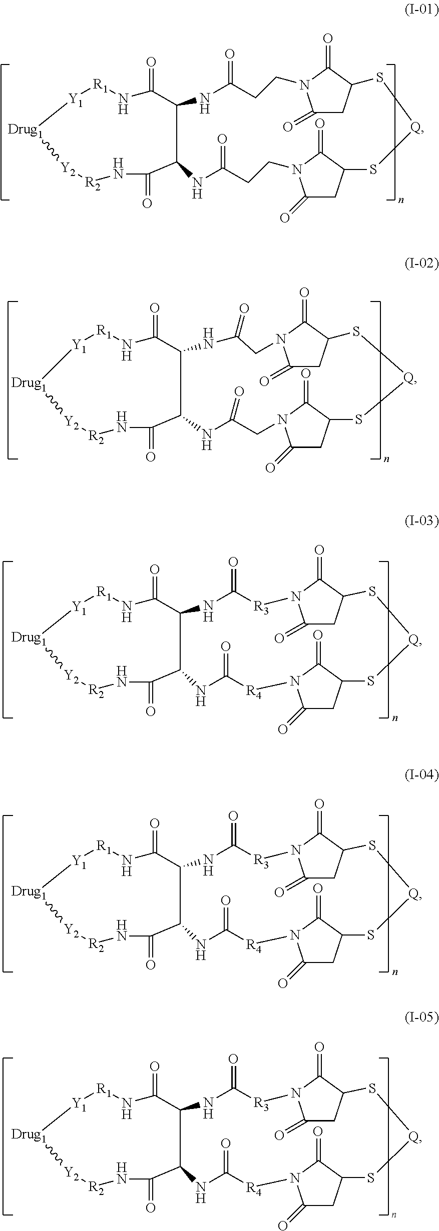

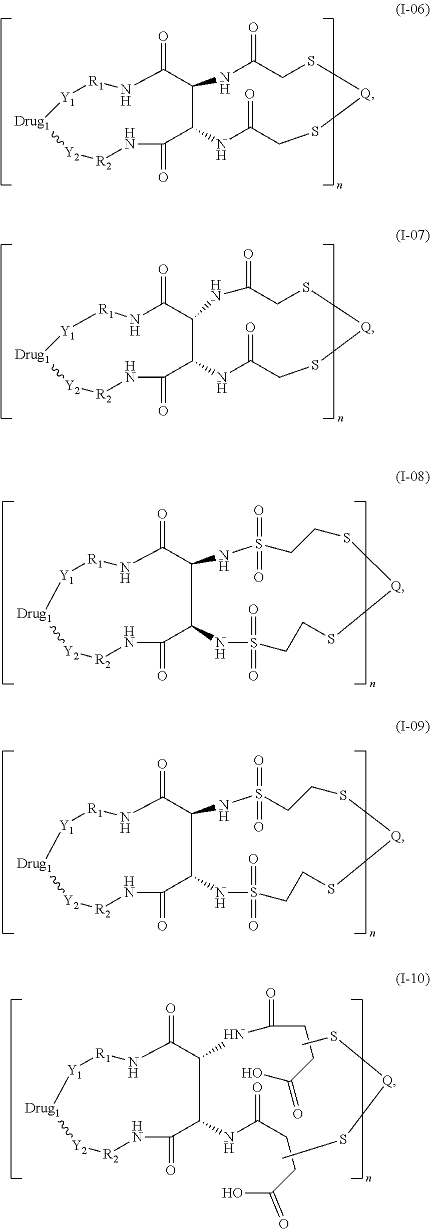

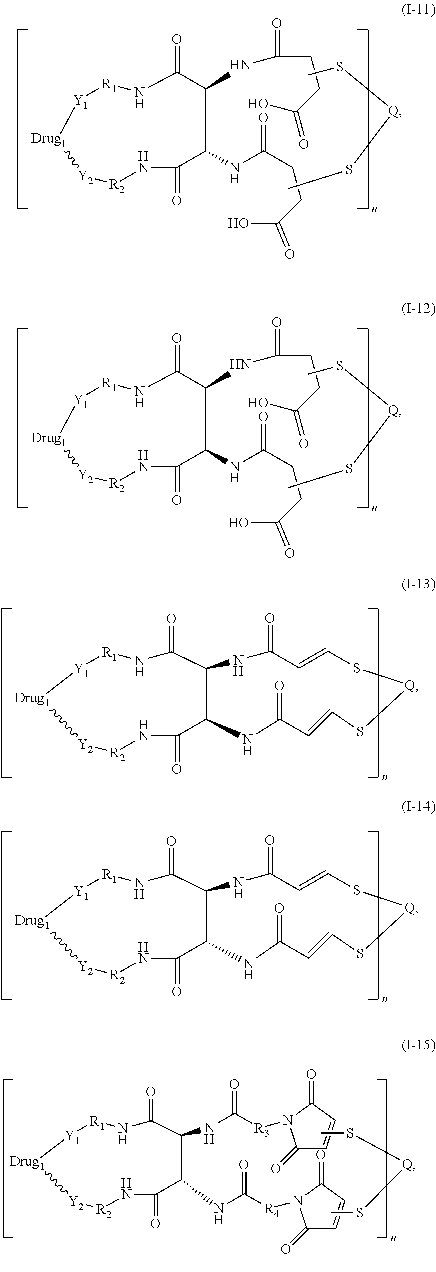

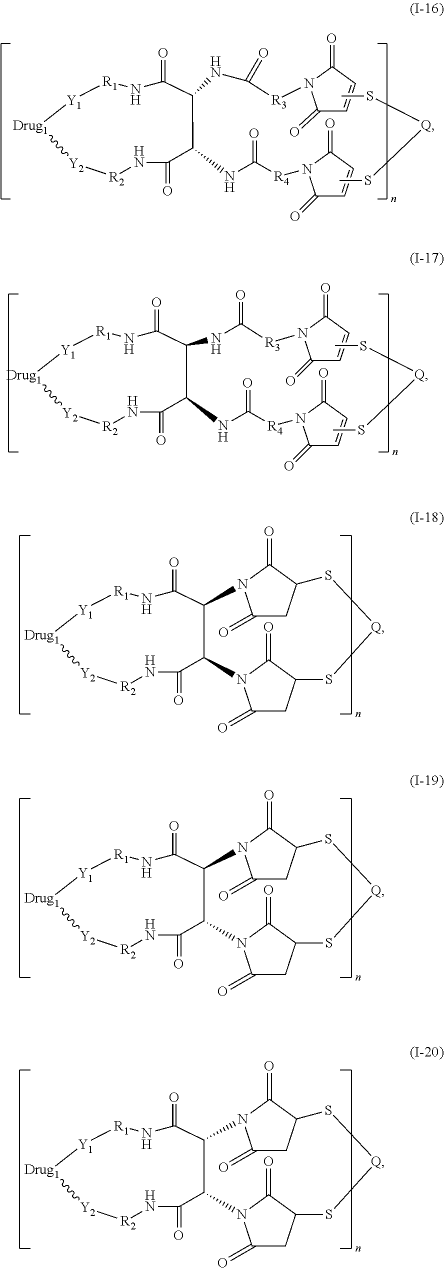

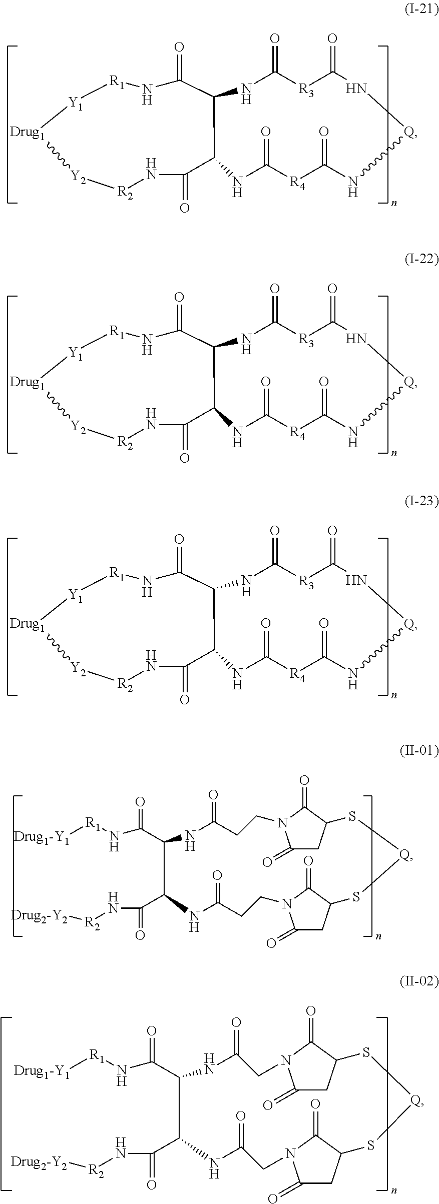

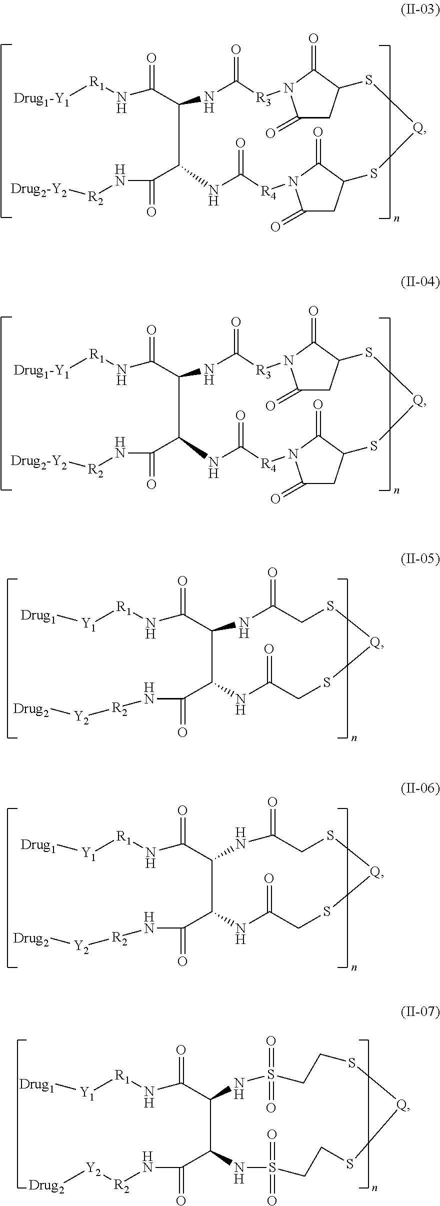

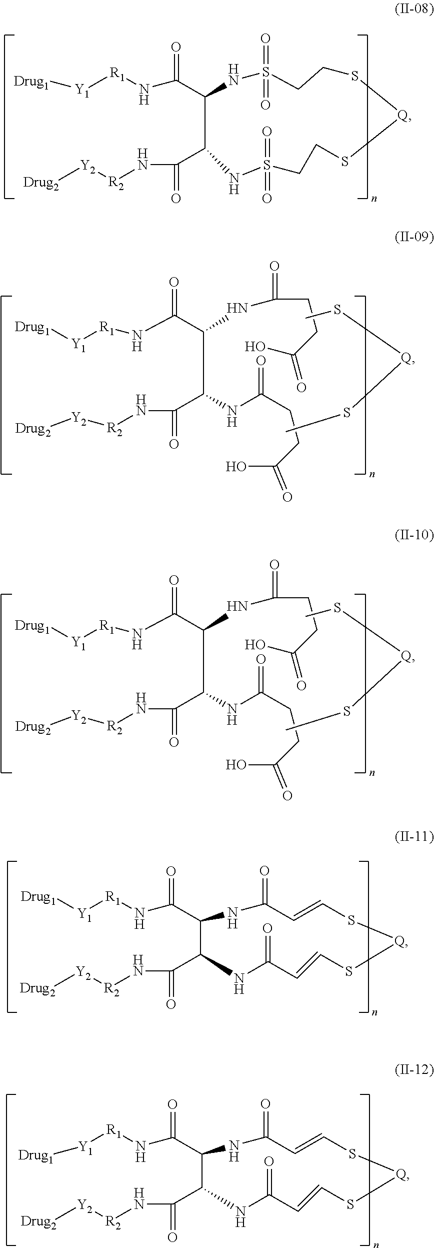

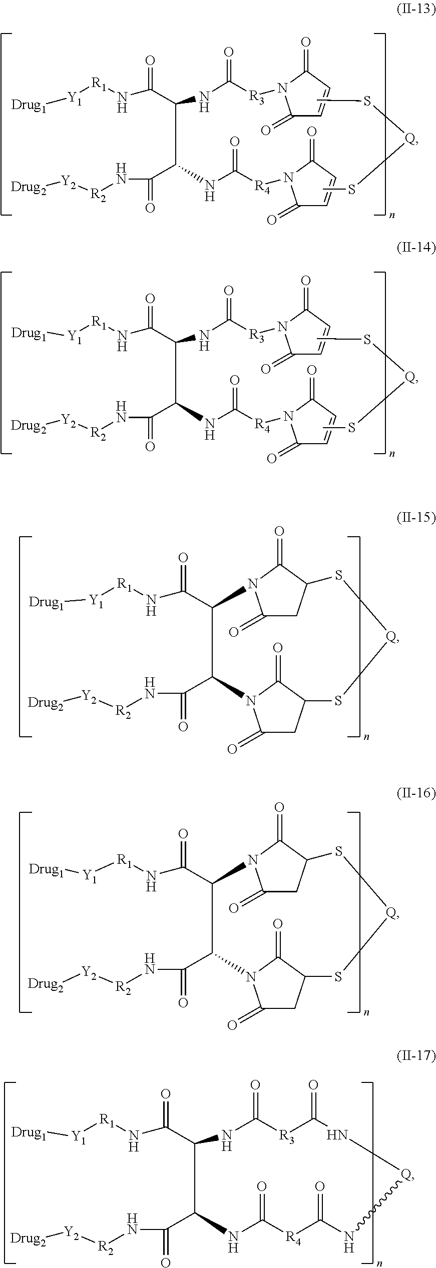

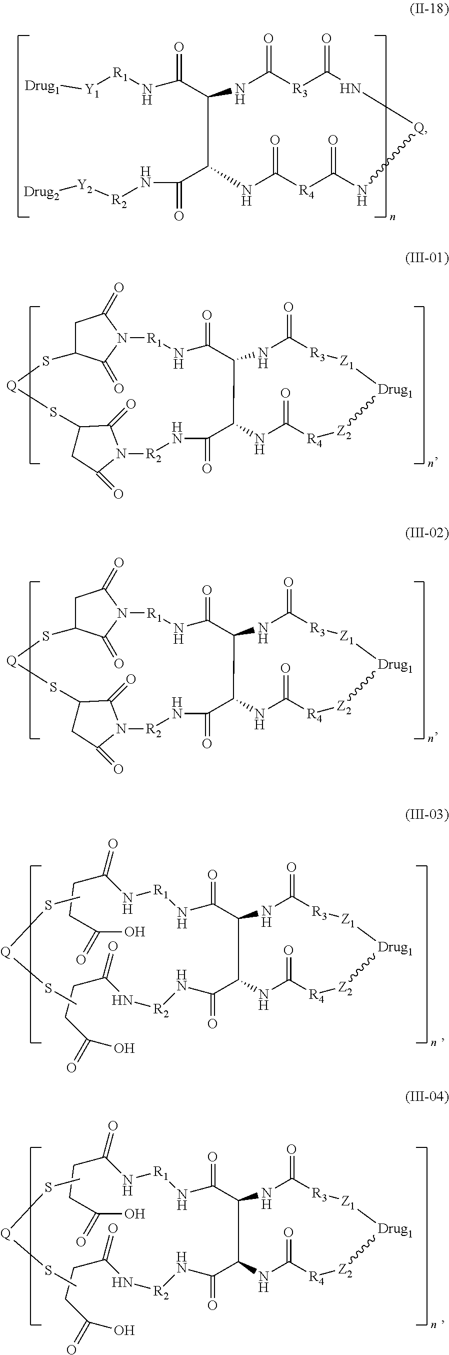

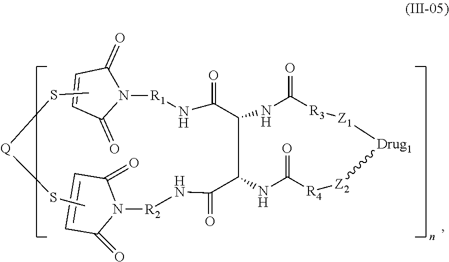

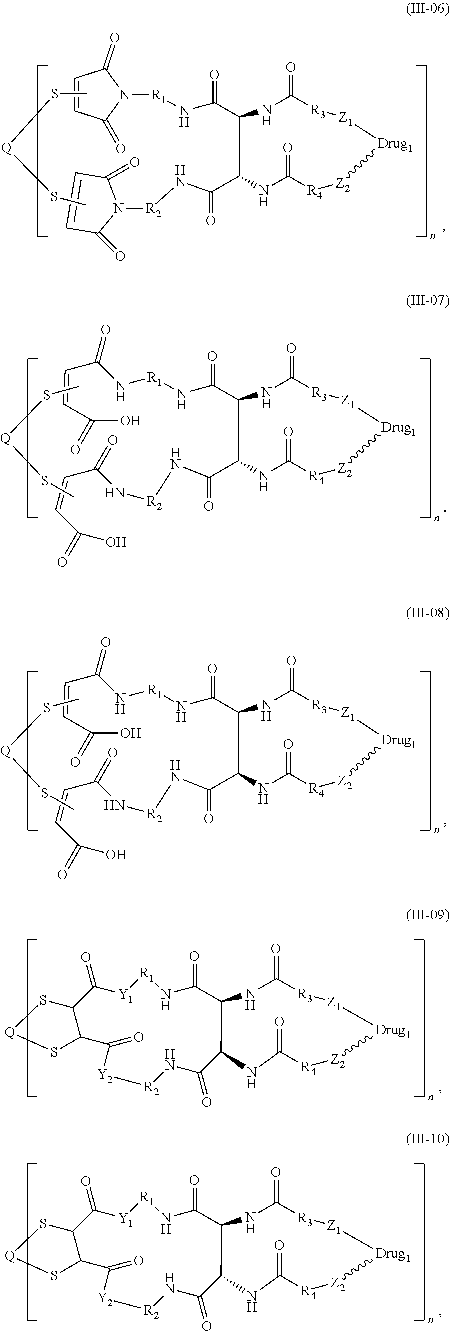

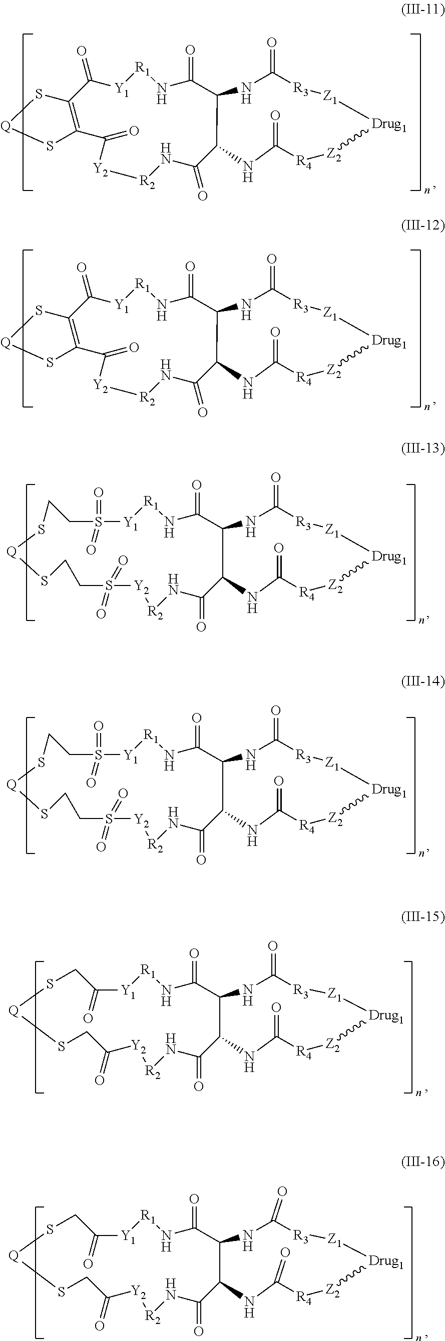

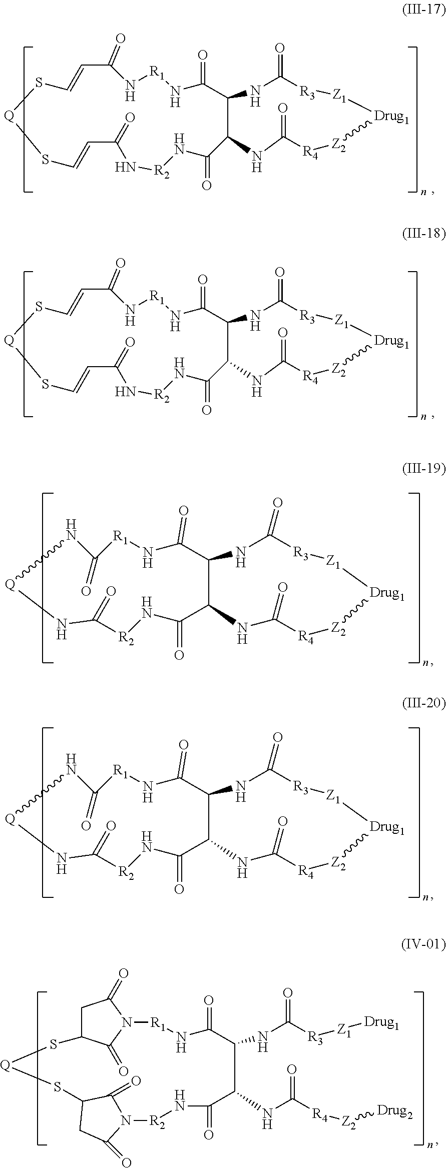

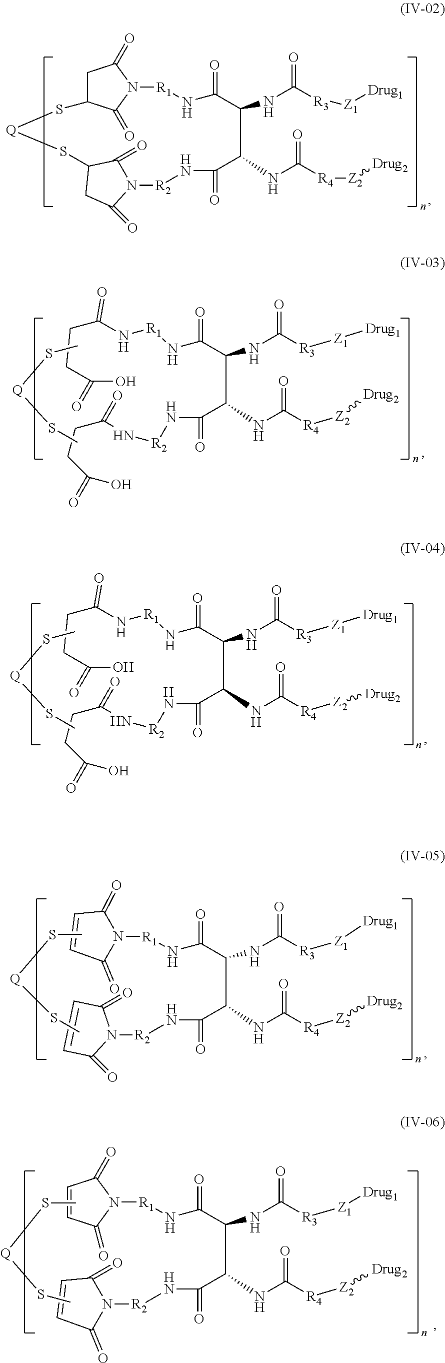

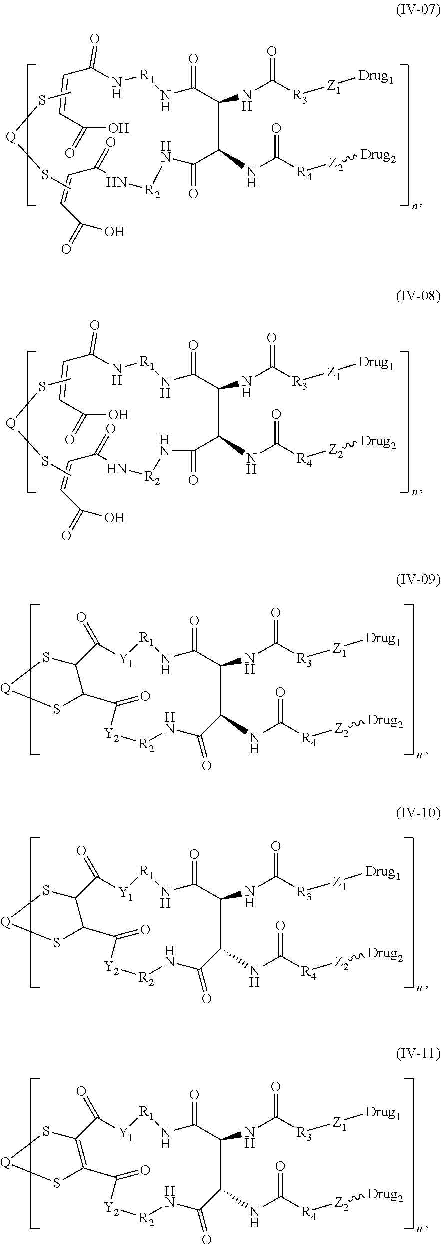

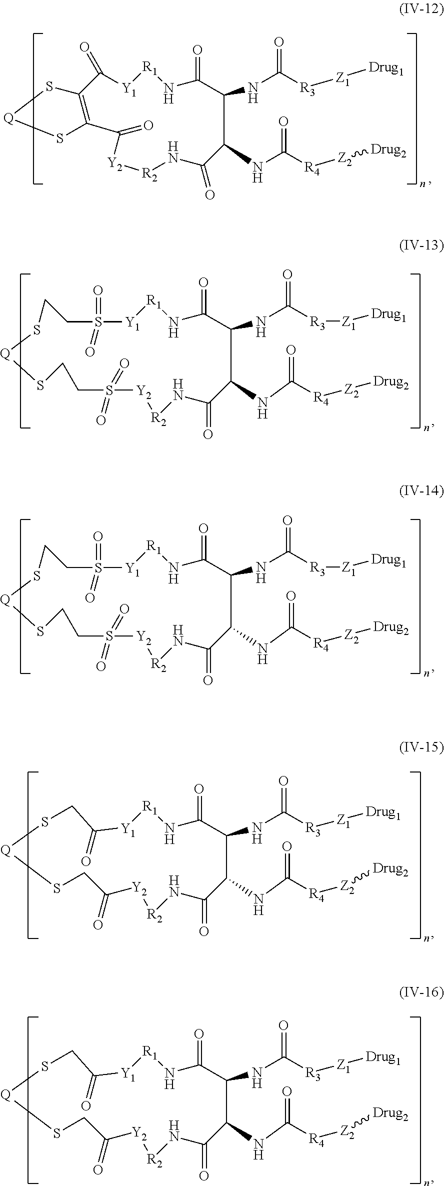

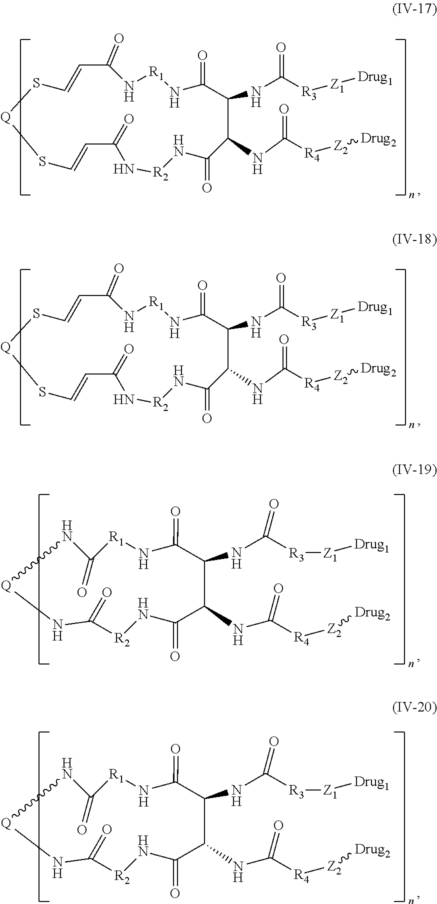

wherein “----”, “ [symbol] ” Q, X₁, X₂, Y₁, Y₂, R₁, R₂, R₃, R₄, R₅, R_{5′}, Z₁, Z₂, Drug₁ and Drug₂ are defined the same above. In addition, one of Drug₁ and Drug₂ can be independently absent but may not be absent at the same time.

The Preparation of the Conjugates of Drugs to a Cell Binding Molecules Via a Bis-Linkage Containing 2,3-Diaminosuccinyl Group

The preparation of the conjugates of drugs to a cell binding molecules of the present invention and the synthetic routes to produce the conjugates via bis-linkage are shown in FIGS. 1-27 and experimental examples.

In another aspect, this invention provides a readily-reactive bis-linker of Formula (V), (VI), (VII) and (VIII) containing 2,3-diaminosuccinyl group below, wherein two or more residues of a cell-binding molecule can simultaneously or sequentially react them to form Formula (I), (II), (III) and (IV) above:

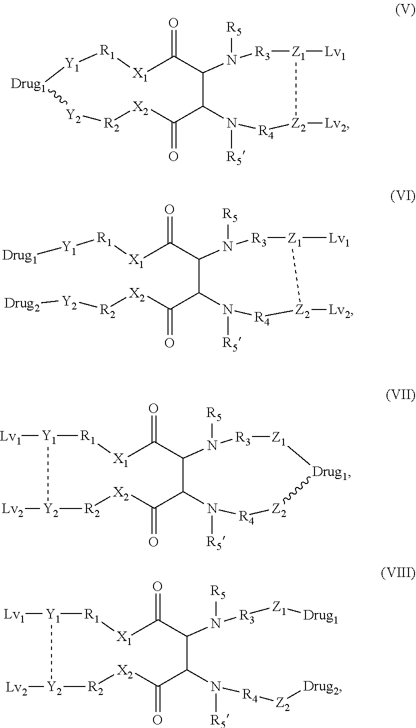

wherein:

“-----” is optionally either a single bond, or a double bond, or a triple bond, or can optionally be absent; It provided that when ----- represents a triple bond, Lv₁ and Lv₂ are absent;

“ [symbol] ”, “ [symbol] ”, Drug₁, Drug₂, n, X₁, X₂, Y₁, Y₂, R₁, R₂, R₃, R₄, R₅, R_{5′}, Z₁, and Z₂ are defined the same as in Formula (I)-(IV);

Lv₁ and Lv₂ represent the same or different leaving group that can be reacted with a thiol, amine, carboxylic acid, selenol, phenol or hydroxyl group on a cell-binding molecule. Lv₁ and Lv₂ are independently selected from OH; F; Cl; Br; I; nitrophenol; N-hydroxysuccinimide (NHS); phenol; dinitrophenol; pentafluorophenol; tetrafluorophenol; difluorophenol; mono-fluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions, or for Mitsunobu reactions. The examples of condensation reagents are: EDC (N-(3-Dimethylaminopropyl)-N′-ethylcarbodiimide), DCC (Dicyclohexyl-carbodiimide), N,N′-Diisopropylcarbodiimide (DIC), N-Cyclohexyl-N′-(2-morpholino-ethyl)carbodiimide metho-p-toluenesulfonate (CMC, or CME-CDI), 1,1′-Carbonyldiimi-dazole (CDI), TBTU (O-(Benzotriazol-1-yl)-N,N,N′,N′-tetramethyluronium tetrafluoroborate), N,N,N′,N′-Tetramethyl-O-(1H-benzotriazol-1-yl)-uronium hexafluorophosphate (HBTU), (Benzotriazol-1-yloxy)tris(dimethylamino)-phosphonium hexafluorophosphate (BOP), (Benzotriazol-1-yloxy)tripyrrolidinophosphonium hexafluorophosphate (PyBOP), Diethyl cyanophosphonate (DEPC), Chloro-N,N,N′,N′-tetramethylformamidiniumhexafluorophosphate, 1-[Bis(dimethylamino)methylene]-1H-1,2,3-triazolo[4,5-b]pyridinium 3-oxid hexafluorophosphate (HATU), 1-[(Dimethylamino)(morpholino)methylene]-1H-[1,2,3]triazolo[4,5-b]pyridine-1-ium 3-oxide hexafluoro-phosphate (HDMA), 2-Chloro-1,3-dimethyl-imidazolidinium hexafluorophosphate (CIP), Chlorotripyrrolidinophosphonium hexafluorophosphate (PyCloP), Fluoro-N,N,N′,N′-bis(tetramethylene)formamidinium hexafluorophosphate (BTFFH), N,N,N′,N′-Tetramethyl-S-(1-oxido-2-pyridyl)thiuronium hexafluorophosphate, 0-(2-Oxo-1(2H)pyridyl)-N,N,N′,N′-tetramethyluronium tetrafluoroborate (TPTU), S-(1-Oxido-2-pyridyl)-N,N,N′,N′-tetramethylthiuronium tetrafluoroborate, 0-[(Ethoxycarbonyl)-cyanomethylenamino]-N,N,N′,N′-tetramethyluronium hexafluorophosphate (HOTU), (1-Cyano-2-ethoxy-2-oxoethylidenaminooxy) dimethylamino-morpholino-carbenium hexafluorophosphate (COMU), O-(Benzotriazol-1-yl)-N,N,N′,N′-bis(tetramethylene)uronium hexafluorophosphate (HBPyU), N-Benzyl-N′-cyclohexyl-carbodiimide (with, or without polymer-bound), Dipyrrolidino(N-succinimidyl-oxy)carbenium hexafluoro-phosphate (HSPyU), Chlorodipyrrolidinocarbenium hexafluorophosphate (PyClU), 2-Chloro-1,3-dimethylimidazolidinium tetrafluoroborate (CIB), (Benzotriazol-1-yloxy)dipiperidino-carbenium hexafluorophosphate (HBPipU), 0-(6-Chlorobenzotriazol-1-yl)-N,N,N′,N′-tetramethyluronium tetrafluoroborate (TCTU), Bromotris(dimethylamino)-phosphonium hexafluorophosphate (BroP), Propylphosphonic anhydride (PPACA, T3P©), 2-Morpholinoethyl isocyanide (MEI), N,N,N′,N′-Tetramethyl-O—(N-succinimidyl)uronium hexafluorophosphate (HSTU), 2-Bromo-1-ethyl-pyridinium tetrafluoroborate (BEP), O-[(Ethoxycarbonyl)cyano-methylenamino]-N,N,N′,N′-tetra-methyluronium tetrafluoroborate (TOTU), 4-(4,6-Dimethoxy-1,3,5-triazin-2-yl)-4-methylmorpholiniumchloride (MMTM, DMTMM), N,N,N′,N′-Tetramethyl-O—(N-succinimidyl)uronium tetrafluoroborate (TSTU), O-(3,4-Dihydro-4-oxo-1,2,3-benzotriazin-3-yl)-N,N,N′,N′-tetramethyluronium tetrafluoro-borate (TDBTU), 1,1′-(Azodicarbonyl)-dipiperidine (ADD), Di-(4-chlorobenzyl)azodicarboxylate (DCAD), Di-tert-butyl azodicarboxylate (DBAD), Diisopropyl azodicarboxylate (DIAD), Diethyl azodicarboxylate (DEAD). In addition, Lv₁ and Lv₂ can be an anhydride, formed by acid themselves or formed with other C₁˜C₈ acid anhydrides;

Preferably Lv₁ and Lv₂ are independently selected from, a halide (e.g., fluoride, chloride, bromide, and iodide), methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluorophenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or carbon-oxygen), or one of the following structure:

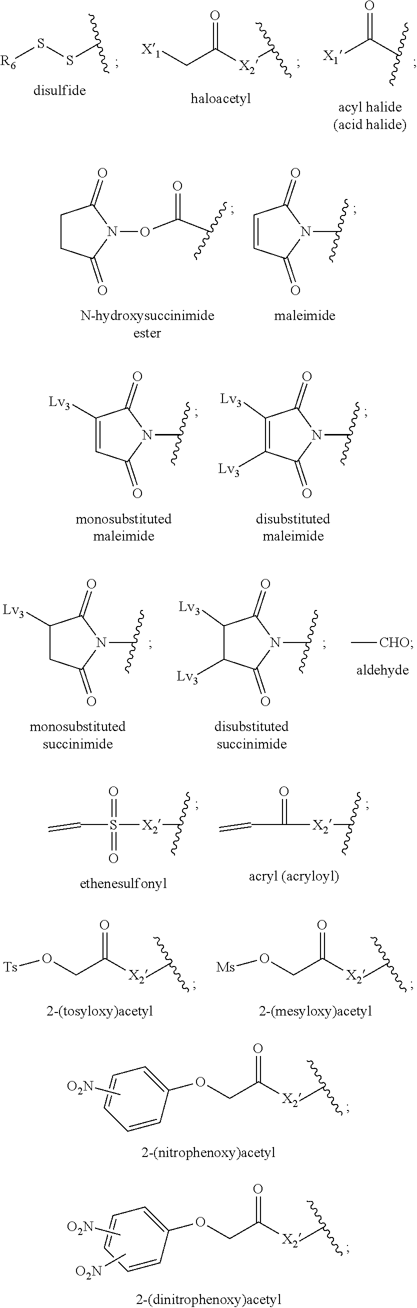

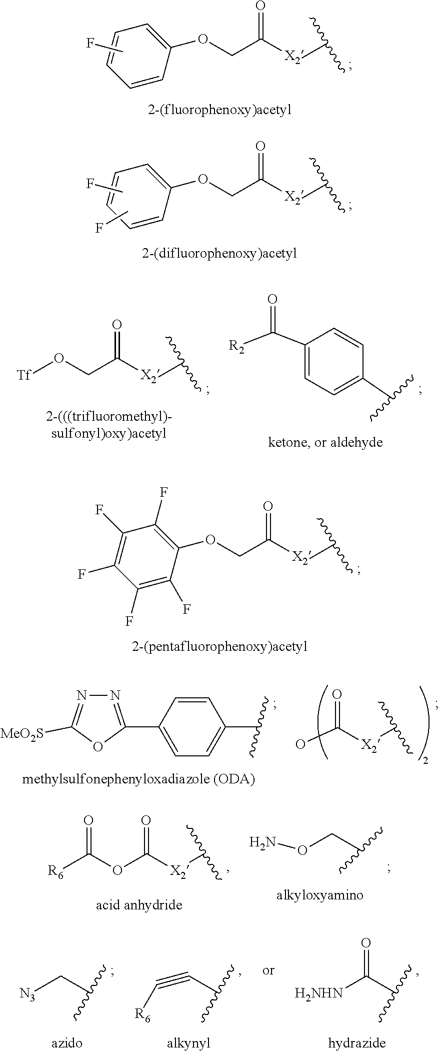

wherein X₁′ is F, Cl, Br, I or Lv₃; X₂′ is O, NH, N(R₁), or CH₂; R₆ is independently H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions;

A preferred stereoisomer of the Formula (I) is presented by the following Formula (Va), (Vb), (Vc), (VIa), (VIb), (VIc), (VIIa), (VIIb), (VIIc), (VIIIa), (VIIIb) and (VIIIc):

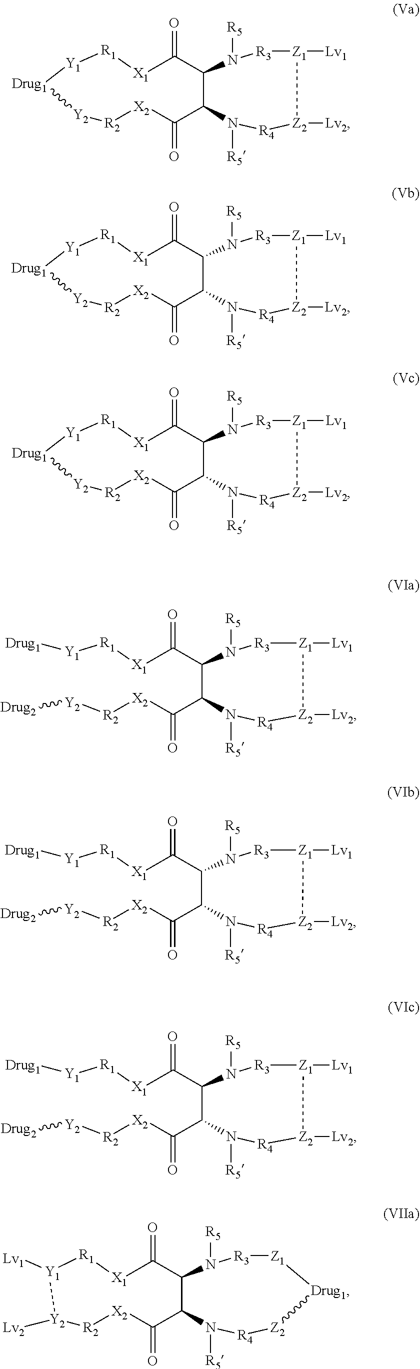

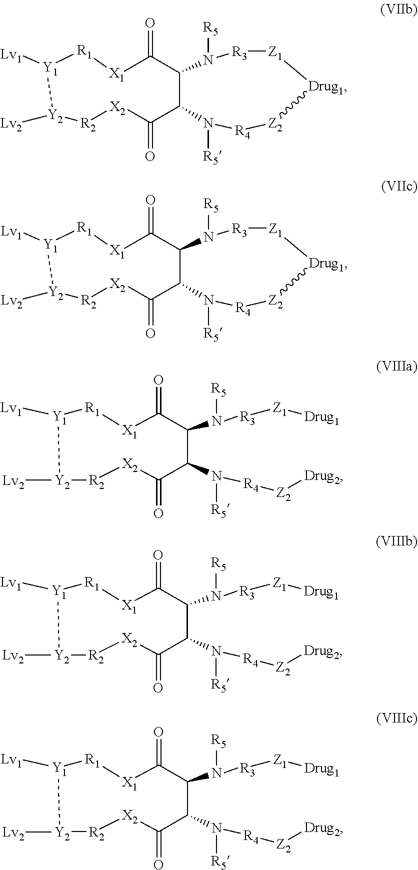

wherein “-----”, Q, X₁, X₂, Y₁, Y₂, R₁, R₂, R₃, R₄, R₅, R_{5′}, Z₁, Z₂, Lv₁, Lv₂, Drug₁ and Drug₂ are defined the same above.

Preferably bis-linkage of the conjugate is further represented by Formula (V-01), (V-02), (V-03), (V-04), (V-05), (V-06), (V-07), (V-08), (V-09), (V-10), (V-11), (V-12), (V-13), (V-14), (V-15), (V-16), (V-17), (V-18), (V-19), (V-20), (V-21), (V-22), (V-23), (VI-01), (VI-02), (VI-03), (VI-04), (VI-05), (VI-06), (VI-07), (VI-08), (VI-09), (VI-10), (VI-11), (VI-12), (VI-13), (VI-14), (VI-15), (VI-16), (VI-17), (VI-18), (VII-01), (VII-02), (VII-03), (VII-04), (VII-05), (VII-06), (VII-07), (VII-08), (VII-09), (VII-10), (VII-11), (VII-12), (VII-13), (VII-14), (VII-15), (VII-16), (VII-17), (VII-18), (VII-19), (VII-20), (VIII-01), (VIII-02), (VIII-03), (VIII-04), (VIII-05), (VIII-06), (VIII-07), (VIII-08), (VIII-09), (VIII-10), (VIII-11), (VIII-12), (VIII-13), (VIII-14), (VIII-15), (VIII-16), (VIII-17), (VIII-18), (VIII-19), and (VIII-20) below:

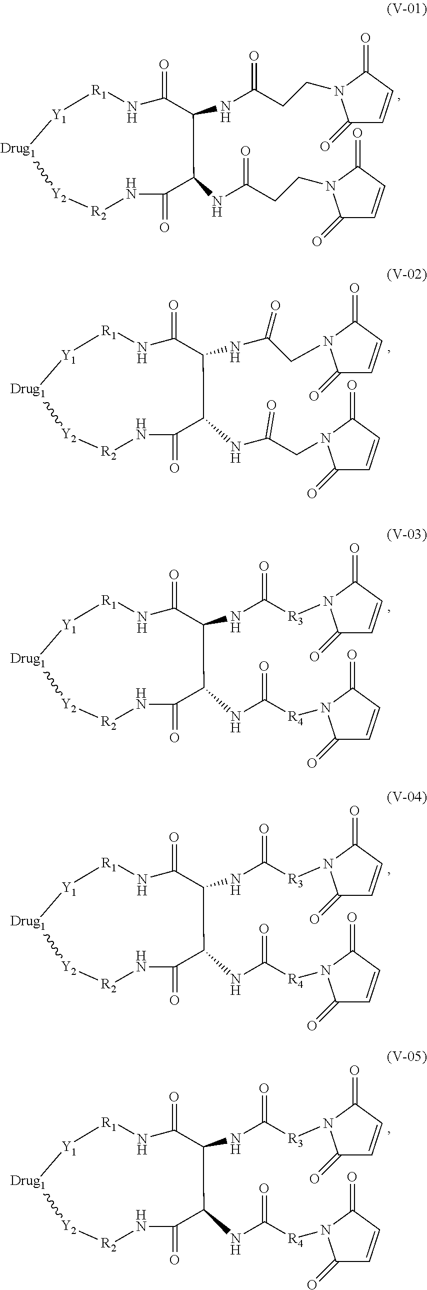

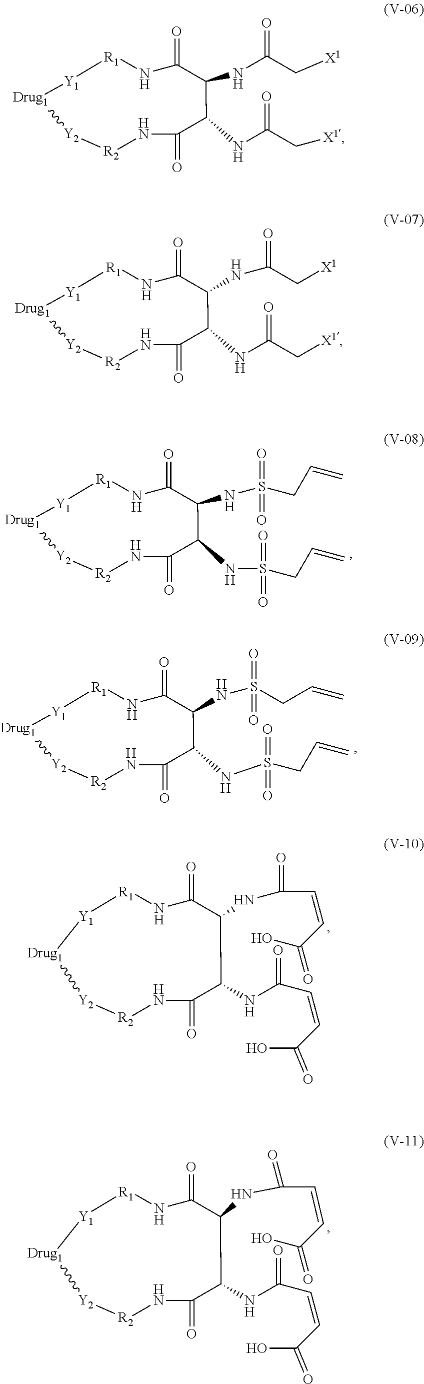

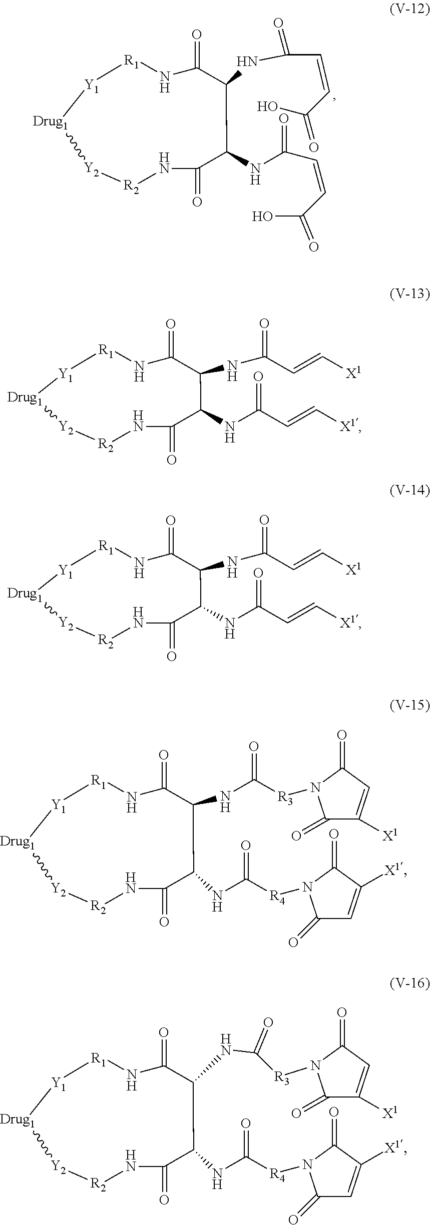

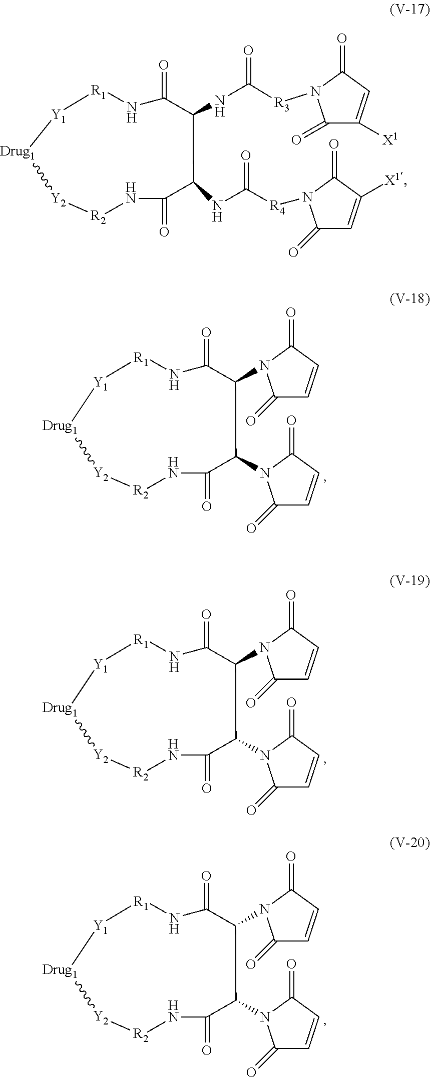

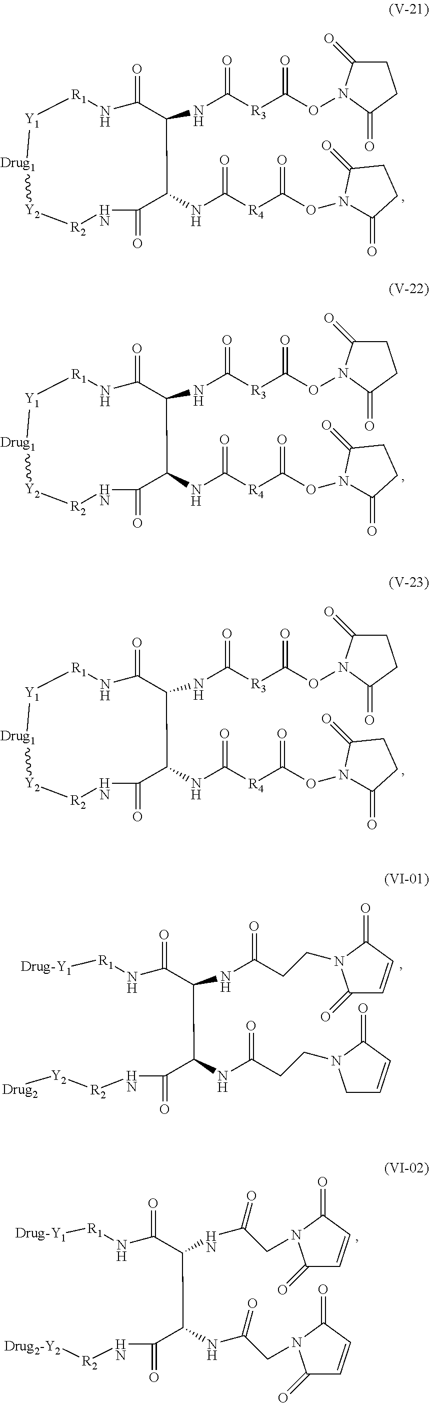

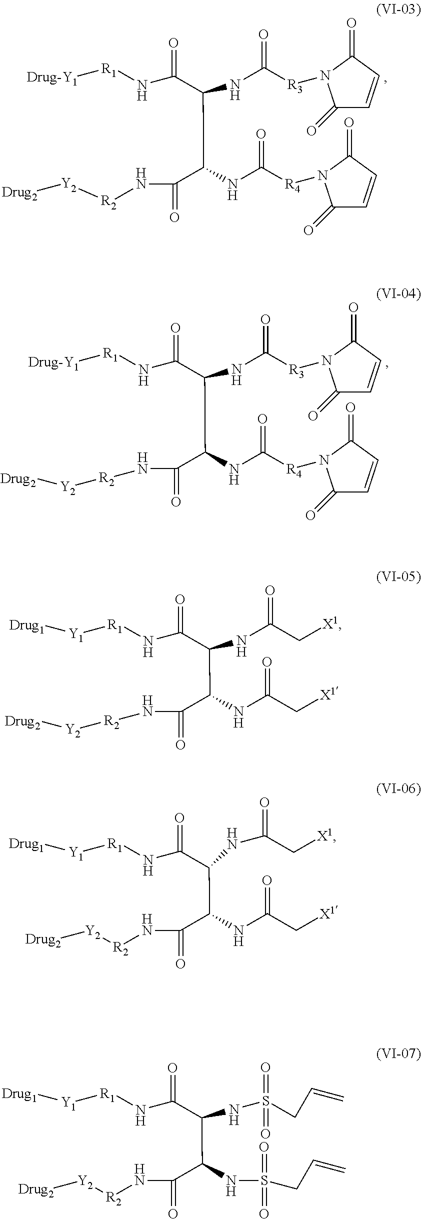

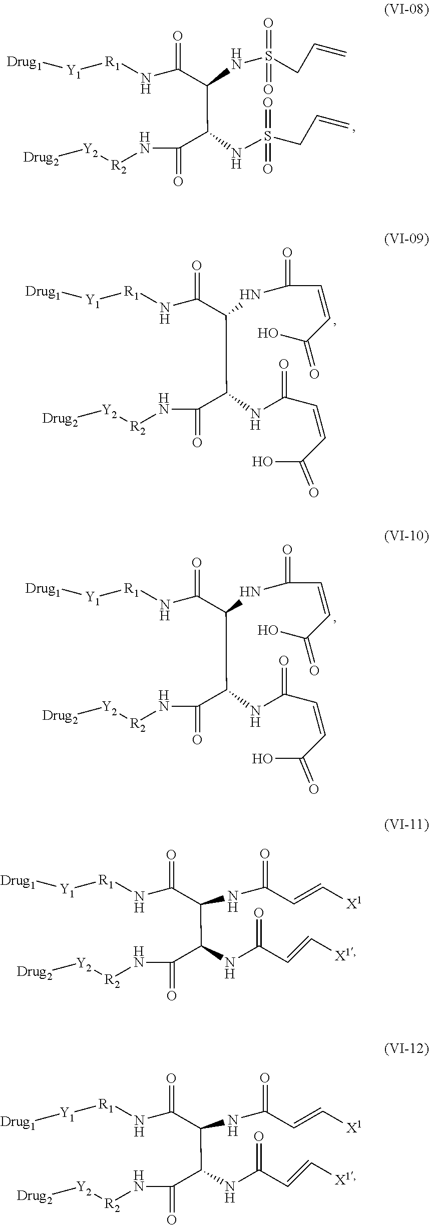

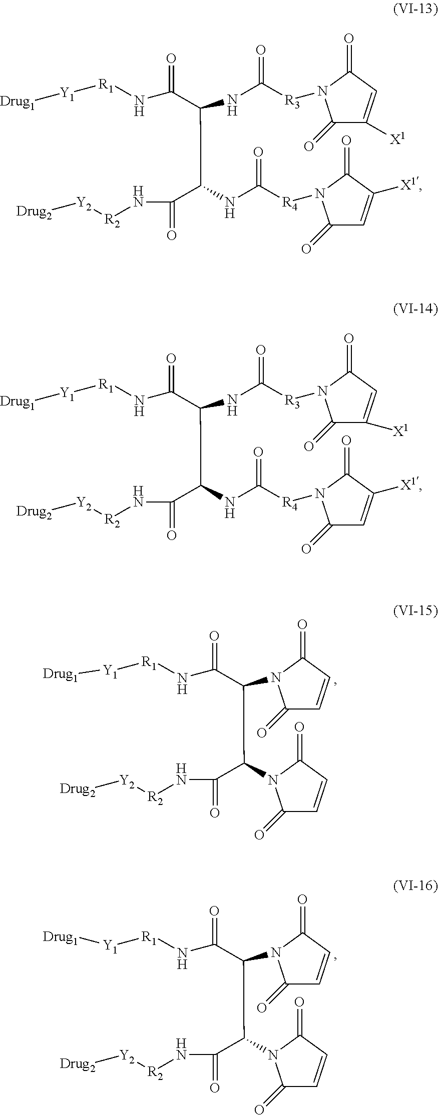

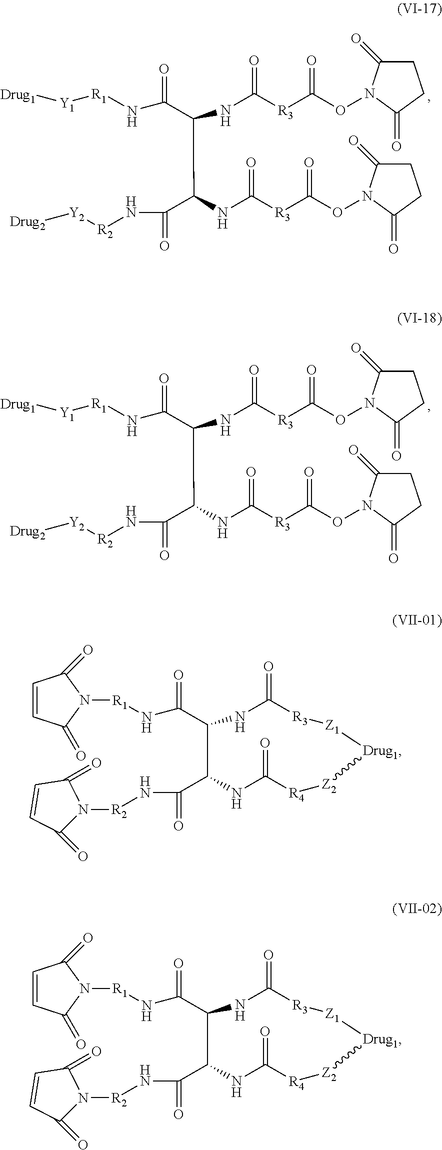

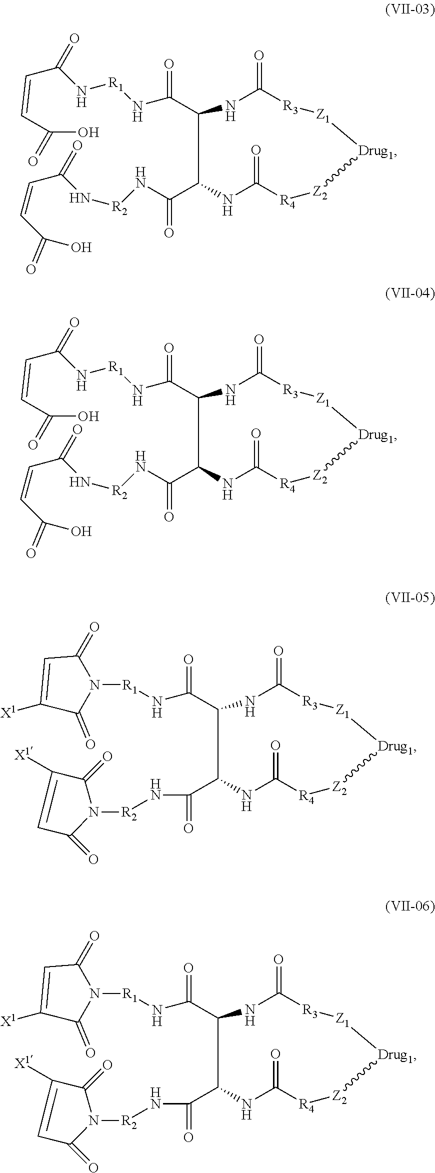

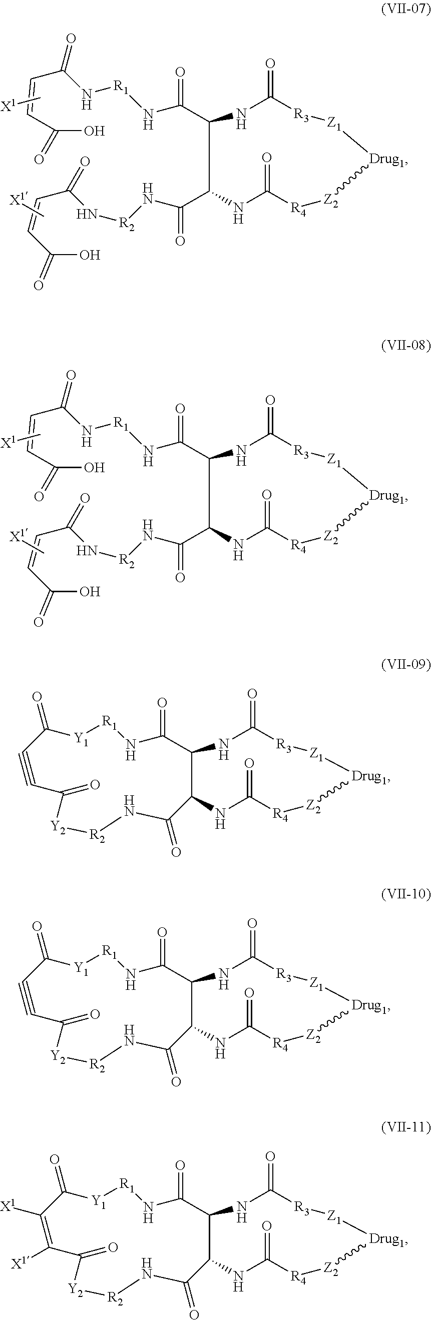

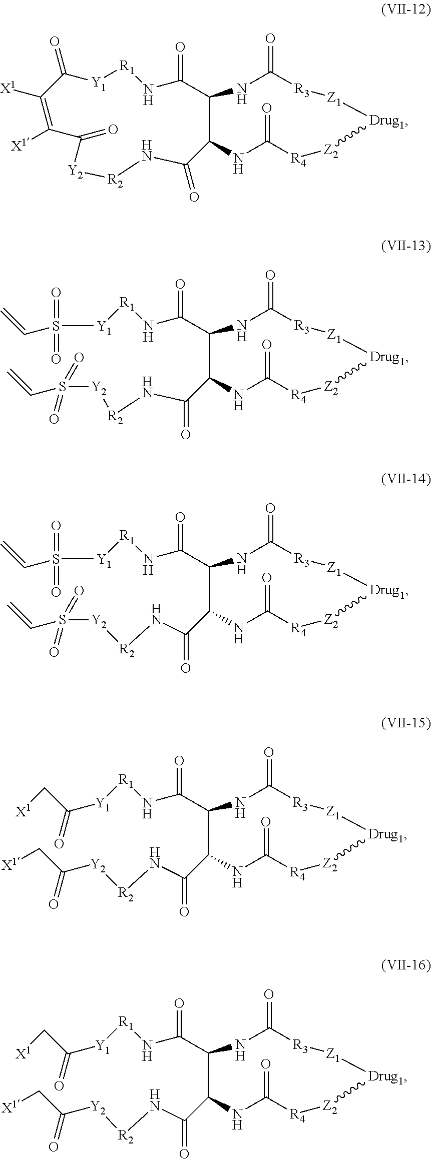

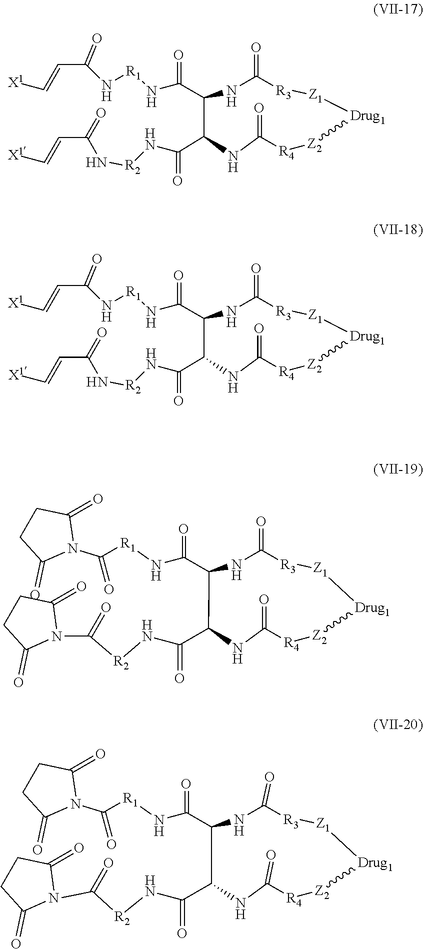

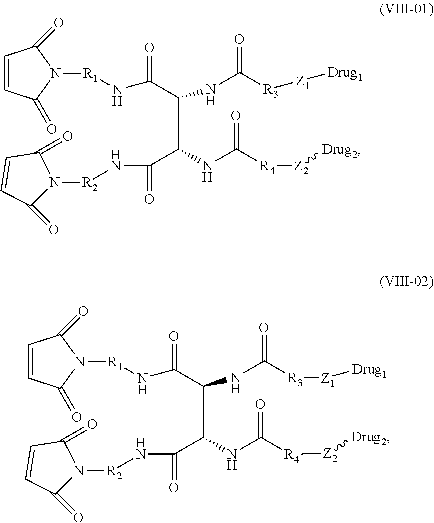

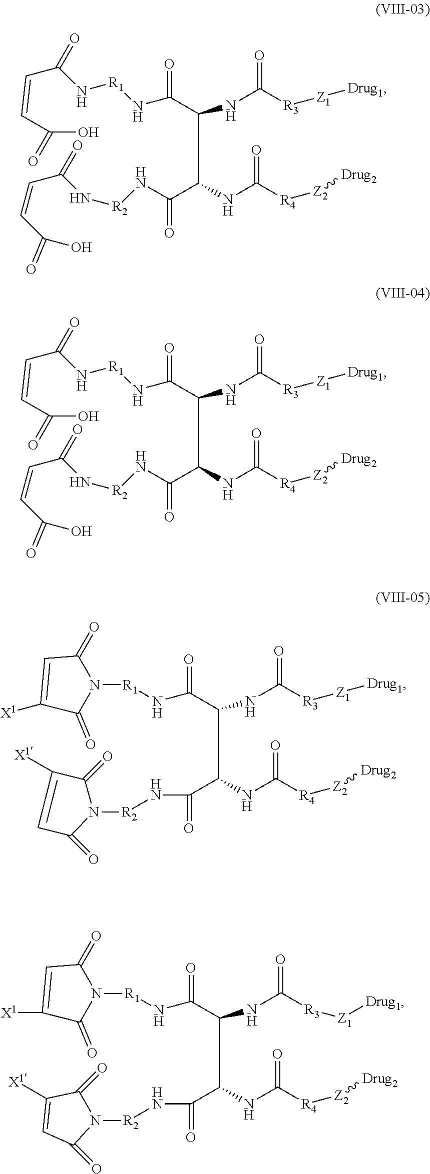

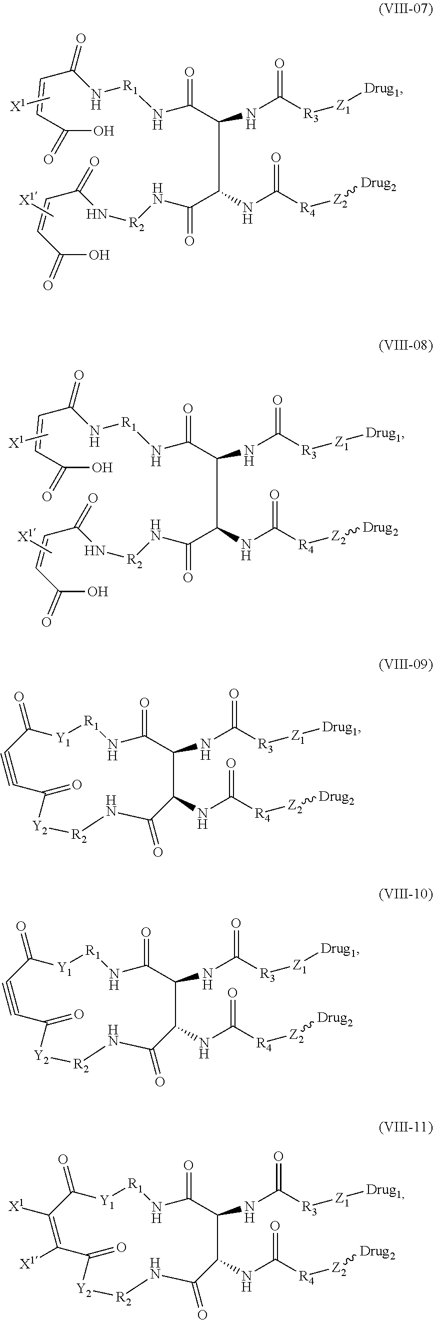

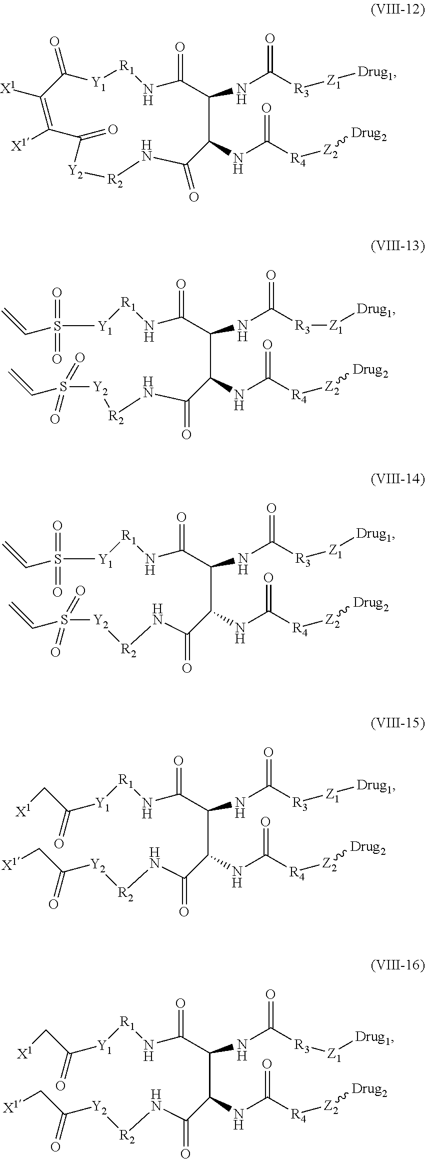

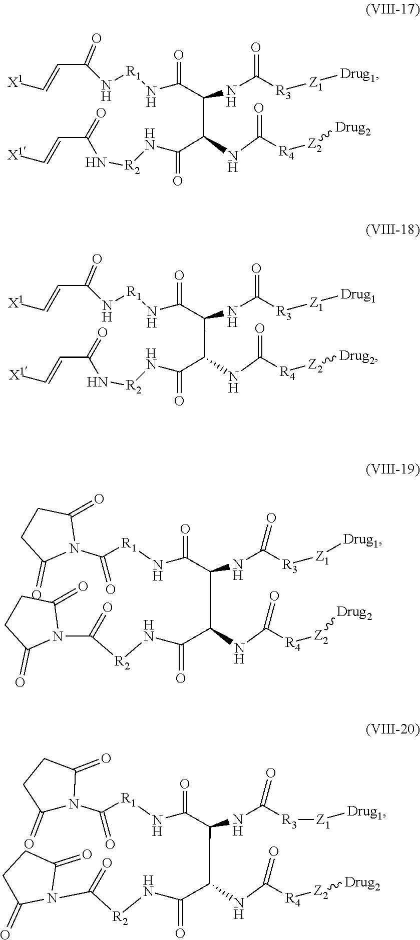

wherein “-----”, “ [symbol] ”, Q, X₁, X₂, Y₁, Y₂, R₁, R₂, R₃, R₄, R₅, R_{5′}, Z₁, Z₂, Drug₁ and Drug₂ are defined the same above; X¹ and X^{1′} are independently H, F, Cl, Br, I, OTs, OMs, OTf, N₃, CHO, —C═CH, —C═C—, ArC(═O)R₁, C(═O)NHNH₂, —O—NH₂, nitrophenol; N-hydroxysuccinimide (NHS); phenol; dinitrophenol; pentafluorophenol; tetrafluorophenol; difluorophenol; mono-fluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; O—NHS (O—N-hydrosuccinimide), O-imidazole, O-triazole, O-tetrazole, O—Ar, O—ArNO₂, O—Ar(NO₂)₂, O—ArF₄, O—ArF₃, O—ArF₅, O—ArF₂, O—ArF, O—ArCl₄, O—ArCl₃, O—ArCl₅, O—ArCl₂, O—ArCl, O—ArSO₃H, O—ArOPO₃H₂, O—Ar(NO₂)COOH, S—Ar(NO₂)₂COOH, O-pyridine, O-nitrophenol, O-dinitrophenol, O-pentafluorophenol, O-tetrafluorophenol, O-trifluorophenol, O-difluorophenol, O-fluorophenol, O-pentachlorophenol, O-tetrachlorophenol, O-trichloro-phenol, O-dichlorophenol, O-chlorophenol, O-pyridine, O-nitropyridine, O-dinitropyridine, O—C₁-C₈ alkyl, O-triflate, O-benzotriazole, S—Ar, S—ArNO₂, S—Ar(NO₂)₂, S—ArF₄, S—ArF₃, S—ArF₅, S—ArF₂, S—ArF, S—ArCl₄, S—ArCl₃, S—ArCl₅, S—ArCl₂, S—ArCl, S—ArSO3H, S—ArOPO₃H₂, S—Ar(NO₂)COOH, S—Ar(NO₂)₂COOH, S-pyridine, S—S-pyridine, S-nitropyridine, S-dinitropyridine, S—C₁-C₈ alkyl, S—S—C₁-C₈ alkyl, S-triflate, S-benzotriazole, wherein Ar is C₃-C₈ aromatic ring; or an intermediate molecule generated with a condensation reagent for peptide coupling reactions, or for Mitsunobu reactions.

In another aspect, this invention provides a readily-reactive bis-linker of Formula (IX) and (X) of following, wherein one or two or more function groups of a cytotoxic molecule can react it simultaneously or sequentially to form Formula (I), (II), (III) or (IV) above:

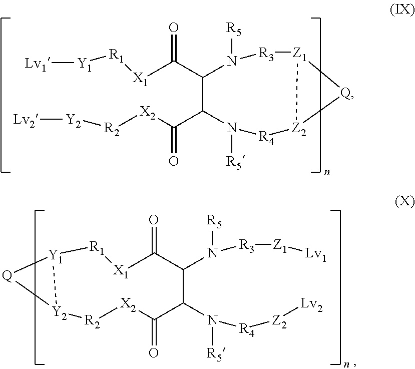

wherein:

“ [symbol] ”, “ [symbol] ”, Q, n, X₁, X₂, Y₁, Y₂, R₁, R₂, R₃, R₄, R₅, R_{5′}, Z₁, and Z₂ are defined the same as in Formula (I)-(IV); and “-----”, Lv₁, Lv₂, Lv₁′ and Lv₂′ are defined the same as Lv₁ and Lv₂ in Formula (V)-(VIII);

Lv₁, Lv₂, Lv₁′ and Lv₂′ are a function group that can independently react with a residue groups of a cytotoxic drug simultaneously or sequentially to form a compound of Formula (I), (II), (III) and (IV) respectively;

In addition, Lv₁, Lv₂, Lv₁′ and Lv₂′ are preferably independently a disulfide substituent, maleimido, haloacetyl, alkoxyamine, azido, ketone, aldehyde, hydrazine, amino, hydroxyl, carboxylate, imidazole, thiol, or alkyne; or a N-hydroxysuccinimide ester, p-nitrophenyl ester, dinitrophenyl ester, pentafluorophenyl ester, pentachlorophenyl ester; tetrafluorophenyl ester; difluorophenyl ester; monofluorophenyl ester; or pentachlorophenyl ester, dichlorophenyl ester, tetrachlorophenyl ester, or 1-hydroxybenzotriazole ester; a triflate, mesylate, or tosylate; 2-ethyl-5-phenylisoxa-zolium-3′-sulfonate; a pyridyldisulfide, or nitropyridyldisulfide; a maleimide, haloacetate, acetylenedicarboxylic group, or carboxylic acid halogenate (fluoride, chloride, bromide, or iodide). Preferably X and Y have one of the following structures:

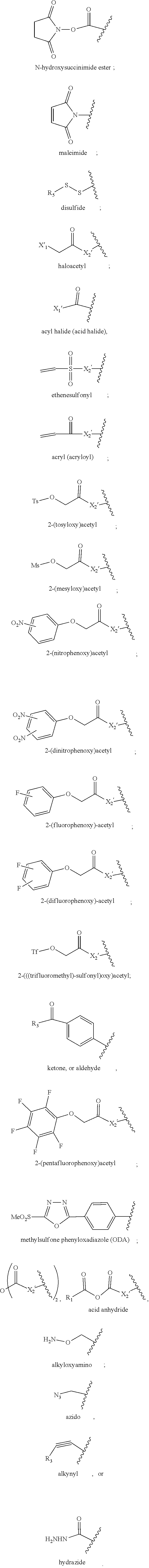

wherein X₁′ is F, Cl, Br, I or Lv₃; X₂′ is O, NH, N(R₁), or CH₂; R₃ and R₅ are H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-yl, phenyloxadiazol-yl (ODA), oxadiazol-yl, or an intermediate molecule generated with a condensation reagent for Mitsunobu reactions, wherein R₁ and R₂ are defined above;

Preferably a bis-linker compound for preparation of the conjugate is further represented by Formula (IX-01), (IX-02), (IX-03), (IX-04), (IX-05), (IX-06), (IX-07), (IX-08), (IX-09), (IX-10), (IX-11), (IX-12), (IX-13), (IX-14), (IX-15), (IX-16), (IX-17), (IX-18), (IX-19), (IX-20), (IX-21), (IX-22), (IX-23), (X-01), (X-02), (X-03), (X-04), (X-05), (X-06), (X-07), (X-08), (X-09), (X-10), (X-11), (X-12), (X-13), (X-14), (X-15), (X-16), (X-17), (X-18), (X-19), and (X-20) below:

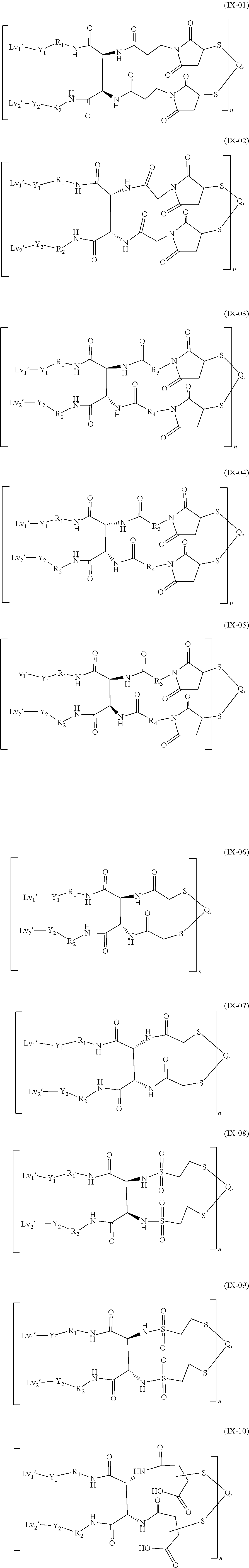

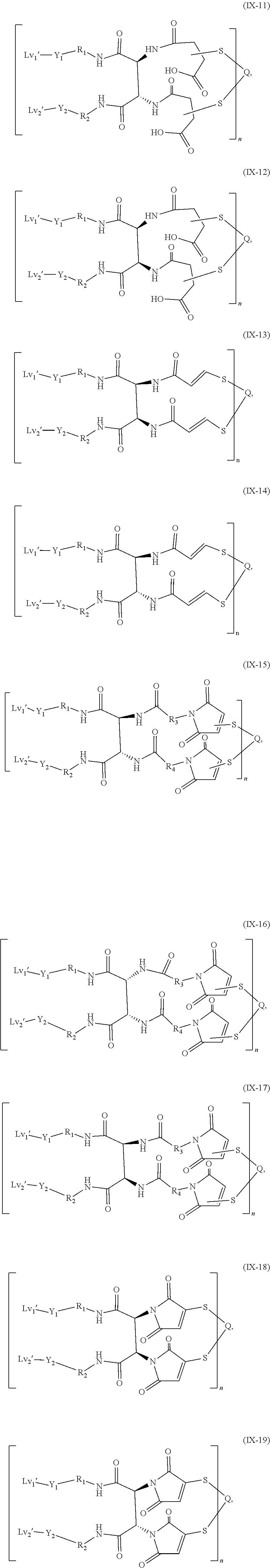

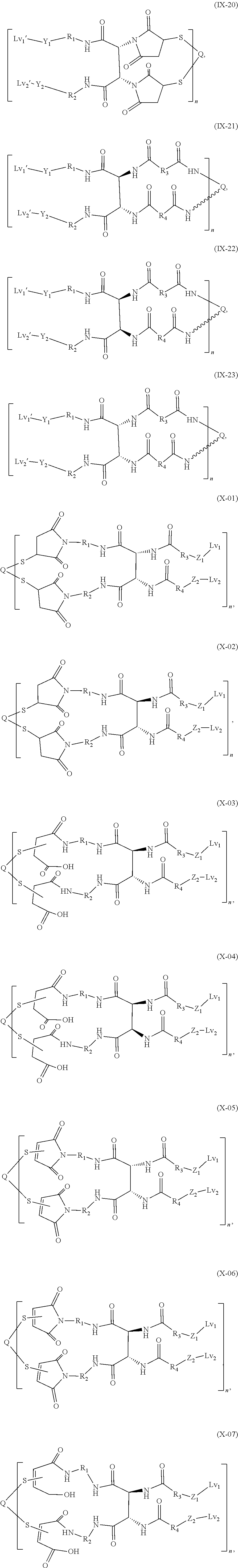

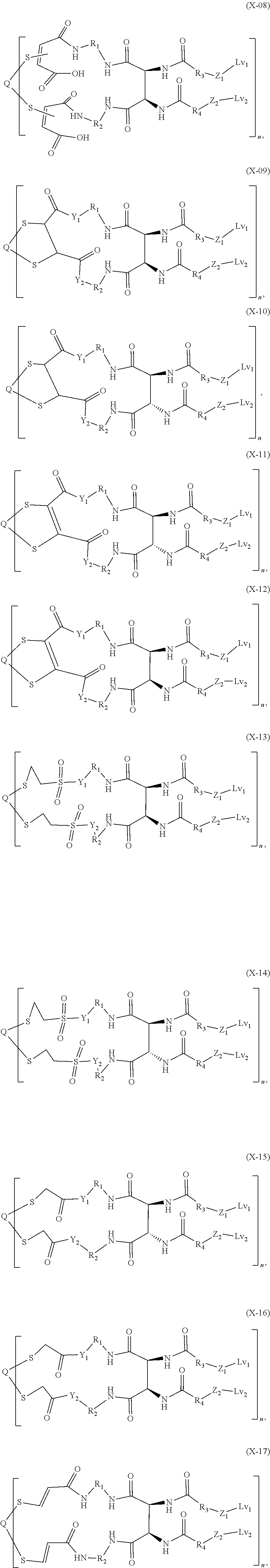

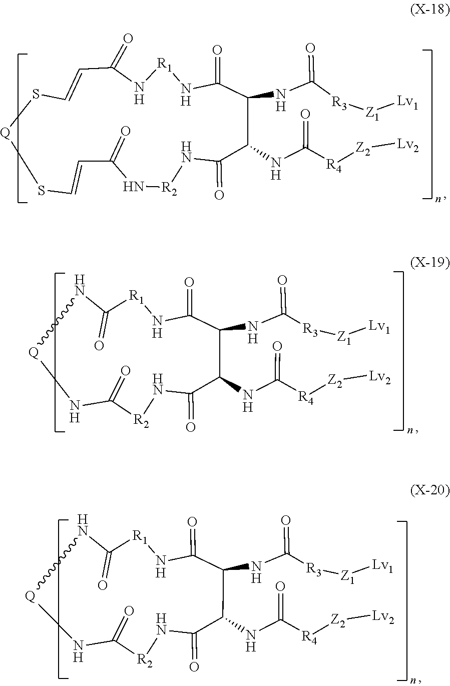

wherein “-----”, “ [symbol] ”, Q, X₁, X₂, Y₁, Y₂, R₁, R₂, R₃, R₄, R₅, R_{5′}, Z₁, Z₂, Lv₁, Lv₂, Lv₁′, and Lv₂′ are defined the same above. In addition, one of Drug₁ and Drug₂ can be independently absent but may not be absent at the same time.

Examples of the functional groups, Lv₁, Lv₂, Lv₁′, and Lv₂′ that enable reaction with the terminal of amine or hydroxyl group of a drug/cytotoxic agent, can be, but not limited to, N-hydroxysuccinimide esters, p-nitrophenyl esters, dinitrophenyl esters, pentafluorophenyl esters, carboxylic acid chlorides or carboxylic acid anhydride; With the terminal of thiol of a cytotoxic agent, can be, as but not limited to, pyridyldisulfides, nitropyridyldisulfides, maleimides, haloacetates, methylsulfonephenyloxadiazole (ODA), carboxylic acid chlorides and carboxylic acid anhydride; With the terminal of ketone or aldehyde, can be, but not limited to, amines, alkoxyamines, hydrazines, acyloxylamine, or hydrazide; With the terminal of azide, can be, as but not limited to, alkyne.

In another aspect, this invention provides a readily-reactive bis-linker of Formula (XI) and (XII) below, wherein a cytotoxic molecule and a cell-binding molecule can react it independently, or simultaneously, or sequentially to form Formula (I)-(IV).

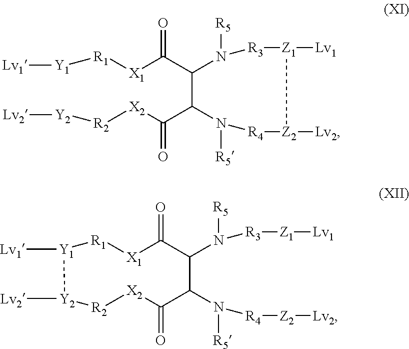

wherein “ [symbol] ”, X₁, X₂, Y₁, Y₂, R₁, R₂, R₃, R₄, R₅, R_{5′}, Z₁, and Z₂ are defined the same as [symbol] in Formula (I)-(IV); and “-----”, Lv₁, Lv₂, Lv₁′ and Lv₂′ are defined the same as Lv₁ and Lv₂ in Formula (V)-(VIII); densation reagent for Mitsunobu reactions, wherein R₁ and R₂ are defined above;

Preferably a bis-linker compound for preparation of the conjugate is further represented by Formula

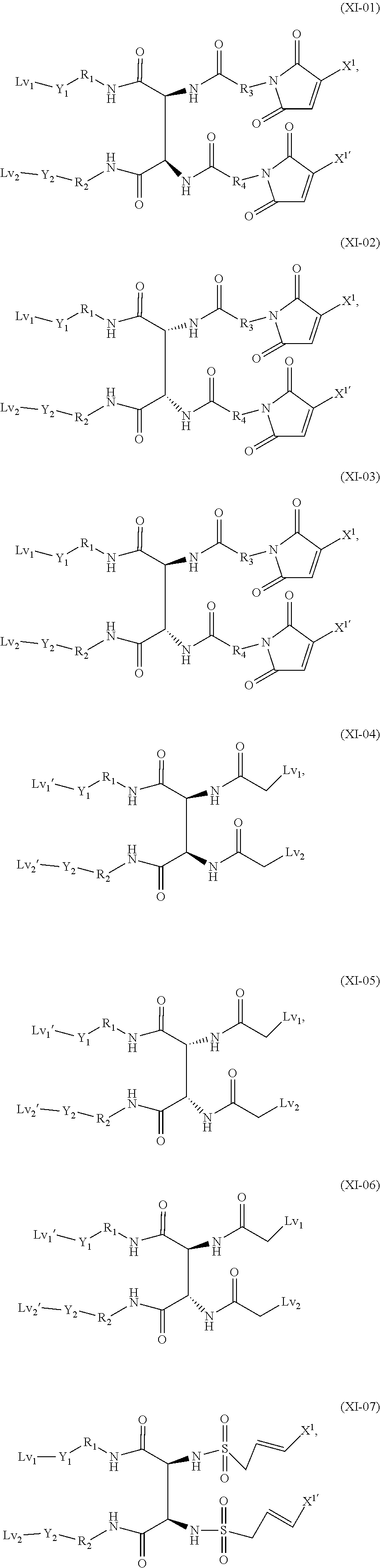

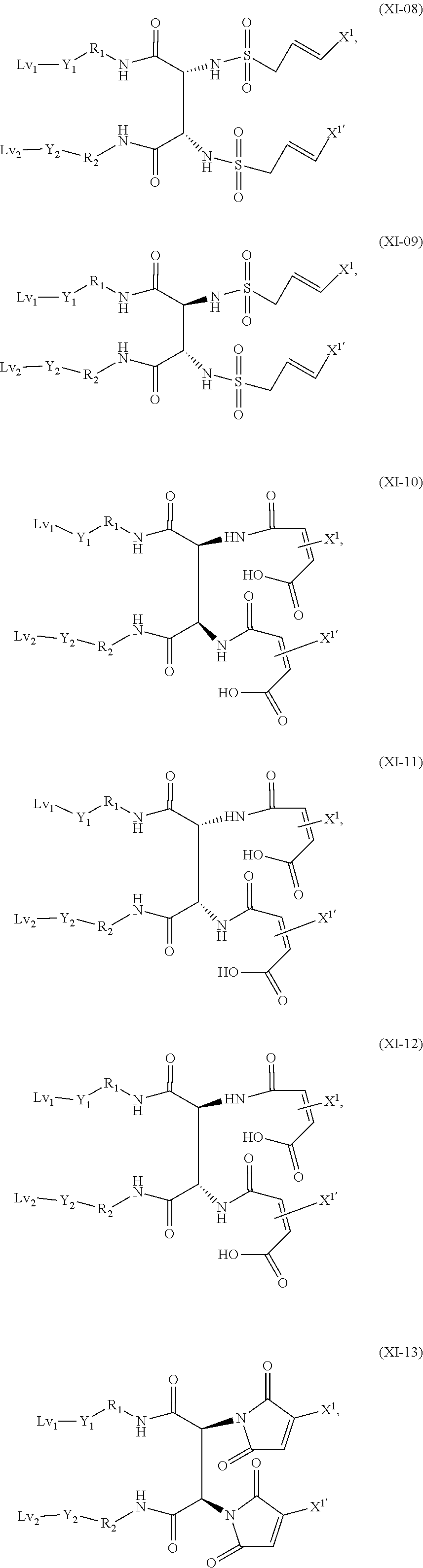

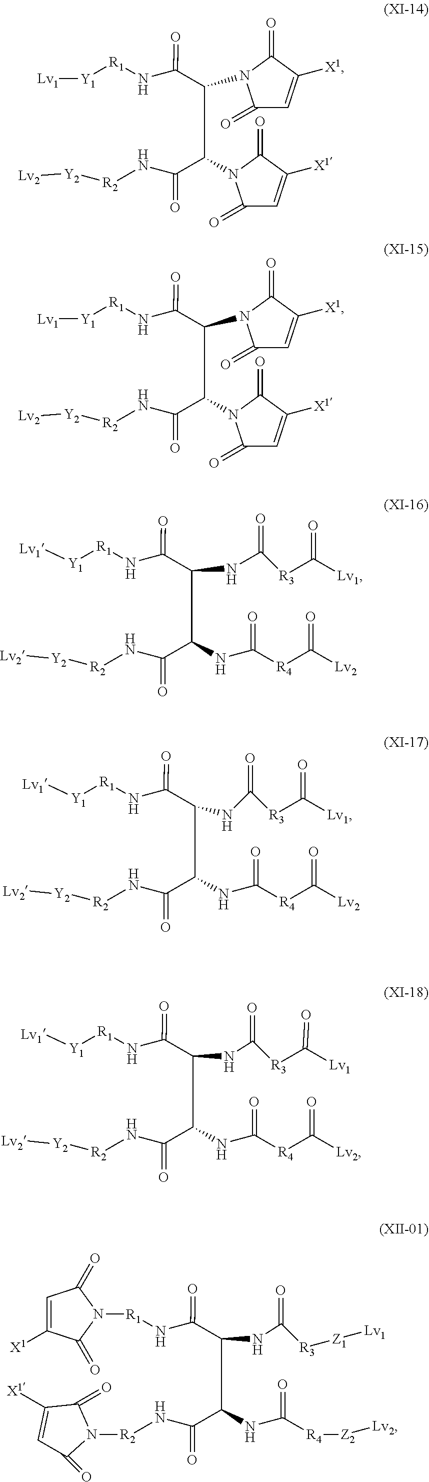

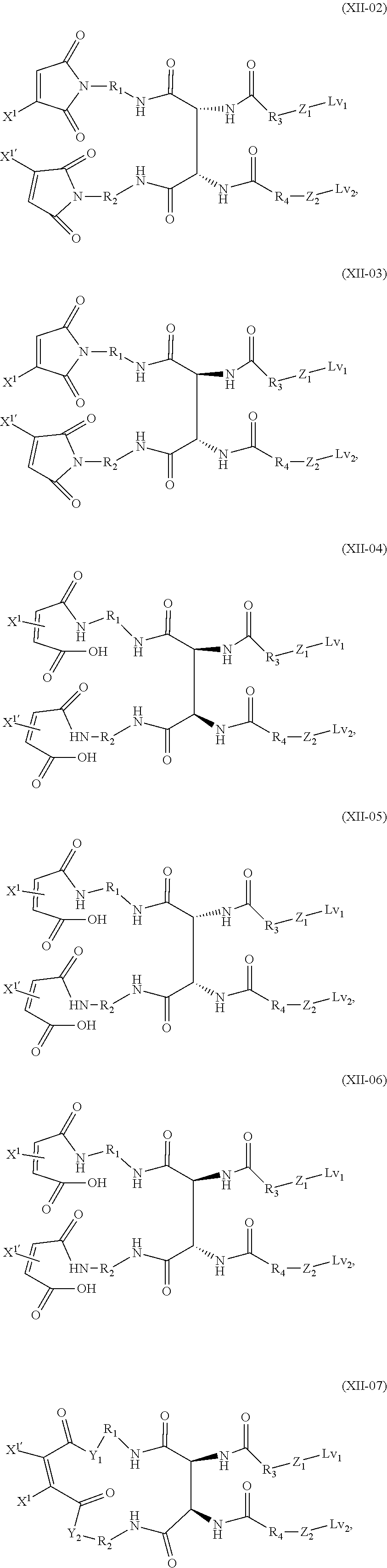

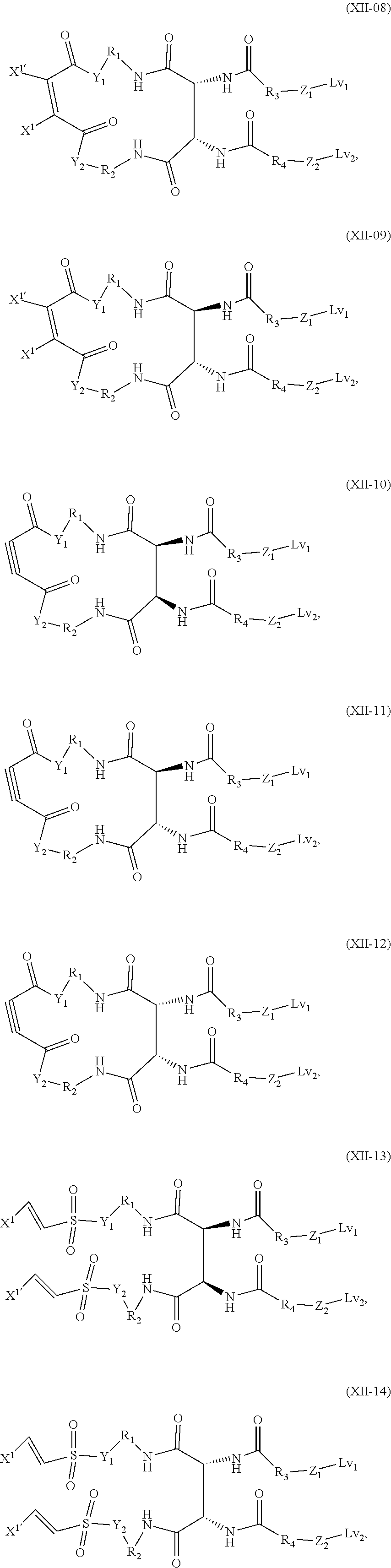

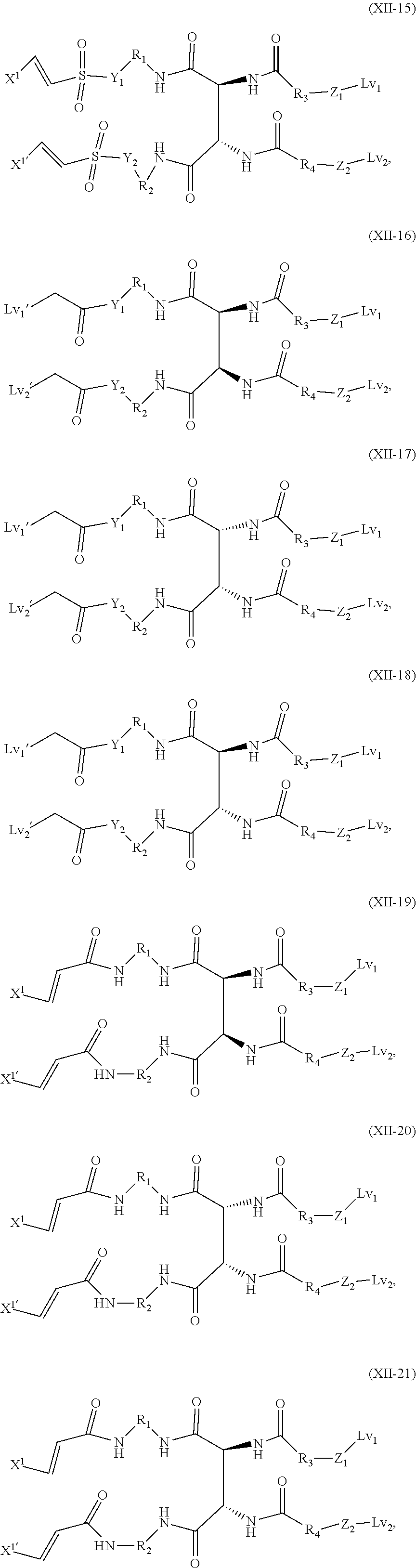

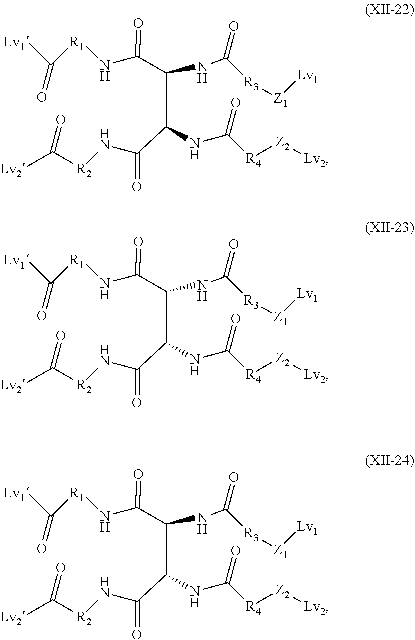

wherein “-----”, “ [symbol] ”, Q, X₁, X₂, Y₁, Y₂, R₁, R₂, R₃, R₄, R₅, R_{5′}, Z₁, Z₂, Lv₁, Lv₂, Lv₁′, Lv₂′, X¹ and X^{1′} are defined the same above;

Some preparations of Formula (I), (II), (III) and (IV) are structurally shown in the FIGS. 1˜26 and in the experimental examples. To synthesize the conjugate of Formula (I), (II), (III) or (IV), in general, two function groups on a drug or on a cell toxicity molecule first reacts sequentially or simultaneously to Lv₁′, Lv₂′, Lv₁ and Lv₂ groups of the linker of Formula (XI) and (XII) in a chemical solvent or in an aqueous media containing 0.1%-99.5% organic solvents or in 100% aqueous media to form a compound of Formula (V), (VI), (VII), or (VIII). Then the compound of Formula (V), (VI), (VII), or (VIII), can be optionally isolated first, or can immediately or simultaneously or sequentially react to two or more residues of a cell binding molecule, preferably a pair of free thiols which are generated through reduction of disulfide bonds of the cell-binding molecule at 0-60° C., pH 5˜9 aqueous media with or without addition of 0˜30% of water mixable (miscible) organic solvents, such as DMA, DMF, ethanol, methanol, acetone, acetonitrile, THF, isopropanol, dioxane, propylene glycol, or ethylene diol to form a conjugate compound of Formula (I), (II), (III) or (IV).

Alternatively, the conjugates of the Formula (I), (II), (III) or (IV) can also be obtained through the first reaction of the linkers of the Formula (XI) or (XII) to two or more residues of a cell binding molecule, preferably a pair of free thiols generated through reduction of disulfide bonds of the cell-binding molecule at 0-60° C., pH 5˜9 aqueous media with or without addition of 0˜30% of water mixable (miscible) organic solvents, to form the modified cell-binding molecule of Formula (IX) or (X). The pairs of thiols are preferred pairs of disulfide bonds which are reduced from the inter chain disulfide bonds of the cell-binding agent by a reduction agent which can be selected from dithiothreitol (DTT), dithioerythritol (DTE), L-glutathione (GSH), tris (2-carboxyethyl) phosphine (TCEP), 2-mercaptoethylamine (β-MEA), or/and beta mercaptoethanol (β-ME, 2-ME) at pH4-9 aqueous media with or without addition of 0˜30% of water mixable (miscible) organic solvents. The reactive groups of Lv₁, Lv₂′, Lv₁ and Lv₂ on Formula (XI) and (XII), which can be independently disulfide, thiol, thioester, maleimido, haloacetyl, azide, 1-yne, ketone, aldehyde, alkoxyamino, triflate, carbonylimidazole, tosylate, mesylate, 2-ethyl-5-phenylisoxazolium-3′-sulfonate, or carboxyl acid esters of nitrophenol, N-hydroxysuccinimide (NHS), phenol; dinitrophenol, pentafluorophenol, tetrafluorophenol, difluorophenol, monofluorophenol, pentachlorophenol, dichlorophenol, tetrachlorophenol, 1-hydroxybenzotriazole, anhydrides, or hydrazide groups, or other acid ester derivatives, can then react to one or two groups on a drug/cytotoxic agent, simultaneously or sequentially at 0-60° C., pH 4˜9.5 aqueous media with or without addition of 0˜30% of water mixable (miscible) organic solvents, to yield a conjugate of the Formula (I), (II), (III) or (IV), after column purification or dialysis. The reactive groups of a drug/cytotoxic agent react to the modified cell-binding molecule of Formula (IX) or (X) in different ways accordingly. For example, a linkage containing disulfide bonds in the cell-binding agent-drug conjugates of Formula (I), (II), (III) or (IV) is achieved by a disulfide exchange between the disulfide bond in the modified cell-binding agent of Formula (IX) or (X) and a drug having a free thiol group; A linkage containing thioether bonds in the cell-binding agent-drug conjugates of Formula (I), (II), (III) or (IV) is achieved by reaction of the maleimido or haloacetyl or ethylsulfonyl modified cell-binding agent of Formula (IX) or (X) and a drug having a free thiol group; A linkage containing a bond of an acid labile hydrazone in the conjugates can be achieved by reaction of a carbonyl group of the drug or compound of Formula (IX) or (X) with the hydrazide moiety on compound of Formula (IX) or (X) or the drug accordingly, by methods known in the art (see, for example, P. Hamann et al., Cancer Res. 53, 3336-34, 1993; B. Laguzza et al., J. Med. Chem., 32; 548-55, 1959; P. Trail et al., Cancer Res., 57; 100-5, 1997); A linkage containing a bond of triazole in the conjugates can be achieved by reaction of a 1-yne group of the drug or compound of Formula (IX) or (X) with the azido moiety on the other counterpart accordingly, through the click chemistry (Huisgen cycloaddition) (Lutz, J-F. et al, 2008, Adv. Drug Del. Rev. 60, 958-70; Sletten, E. M. et al 2011, AccChem. Research 44, 666-76). A linkage containing a bond of oxime in the cell-binding agent-drug conjugates linked via oxime is achieved by reaction of a group of a ketone or aldehyde on the modified cell-binding agent of Formula (IX) or (X) or a drug with a group of oxyamine on a drug or the modified cell-binding agent of Formula (IX) or (X) respectively. A thiol-containing drug can react with the modified cell-binding molecule linker of Formula (IX) or (X) bearing a maleimido, or a haloacetyl, or an ethylsulfonyl substituent at pH 5.5-9.0 in aqueous buffer to give a thioether linkage in cell-binding molecule-drug conjugate of Formula (I), (II), (III) or (IV). A thiol-containing drug can undergo disulfide exchange with a modified linker of Formula (IX) or (X) bearing a pyridyldithio moiety to give a conjugate having a disulfide bond linkage. A drug bearing a hydroxyl group or a thiol group can be reacted with a modified bridge linker of Formula (IX) or (X) bearing a halogen, particularly the alpha halide of carboxylates, in the presence of a mild base, e.g. pH 8.0˜9.5, to give a modified drug bearing an ether or thiol ether linkage. A hydroxyl group on a drug can be condensed with a cross linker of Formula (XI) or (XII) bearing a carboxyl group, in the presence of a dehydrating agent, such as EDC or DCC, to give ester linkage, then the subject drug modified bridge linker of Formula (IX) or (X) undergoes the conjugation with a cell-binding molecule. A drug containing an amino group can condensate with a group of carboxyl ester of NHS, imidazole, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxyben-zotriazole; tosylate; mesylate; 2-ethyl-5-phenylisoxazolium-3′-sulfonate on the cell-binding molecule-linker of Formula ((IX) or (X) to give a conjugate via amide bond linkage.

The synthetic conjugate may be purified by standard biochemical means, such as gel filtration on a Sephadex G25 or Sephacryl S300 column, adsorption chromatography, and ion exchange or by dialysis. In some cases, a small molecule as a cell-binding agent (e.g. folic acid, melanocyte stimulating hormone, EGF etc.) conjugated with a small molecular drugs can be purified by chromatography such as by HPLC, medium pressure column chromatography or ion exchange chromatography.

In order to achieve a higher yield of conjugation reaction of the cytotoxic molecule-bis linker complex of the Formula (V), (VI), (VII), or (VIII) with a pair of free thiols on the cell-binding molecule, preferably on an antibody, a small percentage of water miscible organic solvents, or phase transfer agents, may be required to add to the reaction mixture. To cross-linking reagent (linker) of Formula (V), (VI), (VII), or (VIII) can be first dissolved in a polar organic solvent that is miscible with water, for example in different alcohols, such as methanol, ethanol, and propanol, acetone, acetonitrile, tetrahydrofuran (THF), 1,4-dioxane, dimethyl formamide (DMF), dimethyl acetamide (DMA), or dimethylsulfoxide (DMSO) at a high concentration, for example 1-500 mM. Meanwhile, the cell-binding molecule, such as antibody dissolved in an aqueous buffer pH 4˜9.5, preferably pH 6˜8.5, at 1˜50 mg/ml concentration was treated with 0.5˜20 equivalent of TCEP or DTT for 20 min to 48 hour. After the reduction, DTT can be removed by SEC chromatographic purification. TCEP can be optionally removed by SEC chromatography or ion exchange chromatographies too, or staying in the reaction mixture for the next step reaction without further purification. Furthermore, the reduction of antibodies or the other cell-binding agents with TCEP can be performed along with existing a drug-linker molecule of Formula (V), (VI), (VII), or (VIII), for which the cross-linking conjugation of the cell-binding molecules can be achieved simultaneously along with the TCEP reduction.

The aqueous solutions for the modification of cell-binding agents are buffered between pH 4 and 9, preferably between 6.0 and 7.5 and can contain any non-nucleophilic buffer salts useful for these pH ranges. Typical buffers include phosphate, acetate, triethanolamine HCl, HEPES, and MOPS buffers, which can contain additional components, such as cyclodextrins, Hydroxypropyl-β-cyclodextrin, polyethylene glycols, sucrose and salts, for examples, NaCl and KCl. After the addition of the drug-linker of Formula (V), (VI), (VII), or (VIII) into the solution containing the reduced cell-binding molecules, the reaction mixture is incubated at a temperature of from 4° C. to 45° C., preferably at 15° C.—ambient temperature. The progress of the reaction can be monitored by measuring the decrease in the absorption at a certain UV wavelength, such as at 254 nm, or increase in the absorption at a certain UV wavelength, such as 280 nm, or the other appropriate wavelength. After the reaction is complete, isolation of the modified cell-binding agent can be performed in a routine way, using for example a gel filtration chromatography, an ion exchange chromatography, an adsorptive chromatography or column chromatography over silica gel or alumina, crystallization, preparatory thin layer chromatography, ion (cation or anion) exchange chromatography, or HPLC.

The extent of modification can be assessed by measuring the absorbance of the nitropyridine thione, dinitropyridine dithione, pyridine thione, carboxylamidopyridine dithione and dicarboxyl-amidopyridine dithione group released via UV spectra. For the conjugation without a chromophore group, the modification or conjugation reaction can be monitored by LC-MS, preferably by UPLC-QTOF mass spectrometry, or Capilary electrophoresis-mass spectrometry (CE-MS). The bridge cross-linkers described herein have diverse functional groups that can react with any drugs, preferably cytotoxic agents that possess a suitable substituent. For examples, the modified cell-binding molecules bearing an amino or hydroxyl substituent can react with drugs bearing an N-hydroxysuccinimide (NHS) ester, the modified cell-binding molecules bearing a thiol substituent can react with drugs bearing a maleimido or haloacetyl group. Additionally, the modified cell-binding molecules bearing a carbonyl (ketone or aldehyde) substituent can react with drugs bearing a hydrazide or an alkoxyamine. One skilled in the art can readily determine which linker to use based on the known reactivity of the available functional group on the linkers.

Cell-Binding Agents

The cell-binding molecule, Cb or Q, that comprises the conjugates and the modified cell-binding agents of the present invention may be of any kind presently known, or that become known, molecule that binds to, complexes with, or reacts with a moiety of a cell population sought to be therapeutically or otherwise biologically modified.

The cell binding agents include, but are not limited to, large molecular weight proteins such as, for example, antibody, an antibody-like protein, full-length antibodies (polyclonal antibodies, monoclonal antibodies, dimers, multimers, multispecific antibodies (e.g., a bispecific antibody, trispecific antibody, or tetraspecific antibody); single chain antibodies; fragments of antibodies such as Fab, Fab′, F(ab′)₂, Fᵥ, [Parham, J. Immunol. 131, 2895-902 (1983)], fragments produced by a Fab expression library, anti-idiotypic (anti-Id) antibodies, CDR's, diabody, triabody, tetrabody, miniantibody, a probody, a probody fragment, small immune proteins (SIP), and epitope-binding fragments of any of the above which immunospecifically bind to cancer cell antigens, viral antigens, microbial antigens or a protein generated by the immune system that is capable of recognizing, binding to a specific antigen or exhibiting the desired biological activity (Miller et al (2003) J. of Immunology 170: 4854-61); interferons (such as type I, II, III); peptides; lymphokines such as IL-2, IL-3, IL-4, IL-5, IL-6, IL-7, IL-8, IL-9, IL-10, IL-11, IL-12, IL-13, IL-14, IL-5, IL-16, IL-17, IL-18, IL-19, IL-20, IL-21, IL-22, IL-23, IL-24, IL-25, GM-CSF, interferon-gamma (IFN-7); hormones such as insulin, TRH (thyrotropin releasing hormones), MSH (melanocyte-stimulating hormone), steroid hormones, such as androgens and estrogens, melanocyte-stimulating hormone (MSH); growth factors and colony-stimulating factors such as epidermal growth factors (EGF), granulocyte-macrophage colony-stimulating factor (GM-CSF), transforming growth factors (TGF), such as TGFα, TGFβ, insulin and insulin like growth factors (IGF-I, IGF-II) G-CSF, M-CSF and GM-CSF [Burgess, Immunology Today, 5, 155-8 (1984)]; vaccinia growth factors (VGF); fibroblast growth factors (FGFs); smaller molecular weight proteins, poly-peptide, peptides and peptide hormones, such as bombesin, gastrin, gastrin-releasing peptide; platelet-derived growth factors; interleukin and cytokines, such as interleukin-2 (IL-2), interleukin-6 (IL-6), leukemia inhibitory factors, granulocyte-macrophage colony-stimulating factor (GM-CSF); vitamins, such as folate; apoproteins and glycoproteins, such as transferrin [O'Keefe et al, 260 J. Biol. Chem. 932-7 (1985)]; sugar-binding proteins or lipoproteins, such as lectins; cell nutrient-transport molecules; and small molecular inhibitors, such as prostate-specific membrane antigen (PSMA) inhibitors and small molecular tyrosine kinase inhibitors (TKI), non-peptides or any other cell binding molecule or substance, such as bioactive polymers (Dhar, et al, Proc. Natl. Acad. Sci. 2008, 105, 17356-61); bioactive dendrimers (Lee, et al, Nat. Biotechnol. 2005, 23, 1517-26; Almutairi, et al; Proc. Natl. Acad. Sci. 2009, 106, 685-90); nanoparticles (Liong, et al, ACS Nano, 2008, 2, 1309-12; Medarova, et al, Nat. Med. 2007, 13, 372-7; Javier, et al, Bioconjugate Chem. 2008, 19, 1309-12); liposomes (Medinai, et al, Curr. Phar. Des. 2004, 10, 2981-9); viral capsides (Flenniken, et al, Viruses Nanotechnol. 2009, 327, 71-93).

In general, a monoclonal antibody is preferred as a cell-surface binding agent if an appropriate one is available. And the antibody may be murine, human, humanized, chimeric, or derived from other species.

Production of antibodies used in the present invention involves in vivo or in vitro procedures or combinations thereof. Methods for producing polyclonal anti-receptor peptide antibodies are well-known in the art, such as in U.S. Pat. No. 4,493,795 (to Nestor et al). A monoclonal antibody is typically made by fusing myeloma cells with the spleen cells from a mouse that has been immunized with the desired antigen (Kohler, G.; Milstein, C. (1975). Nature 256: 495-7). The detailed procedures are described in “Antibodies--A Laboratory Manual”, Harlow and Lane, eds., Cold Spring Harbor Laboratory Press, New York (1988), which is incorporated herein by reference. Particularly monoclonal antibodies are produced by immunizing mice, rats, hamsters or any other mammal with the antigen of interest such as the intact target cell, antigens isolated from the target cell, whole virus, attenuated whole virus, and viral proteins. Splenocytes are typically fused with myeloma cells using polyethylene glycol (PEG) 6000. Fused hybrids are selected by their sensitivity to HAT (hypoxanthine-aminopterin-thymine). Hybridomas producing a monoclonal antibody useful in practicing this invention are identified by their ability to immunoreact specified receptors or inhibit receptor activity on target cells.

A monoclonal antibody used in the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques, such as using protein-A affinity chromatography; anion, cation, hydrophobic, or size exclusive chromatographies (particularly by affinity for the specific antigen after protein A, and sizing column chromatography); centrifugation, differential solubility, or by any other standard technique for the purification of proteins.

Media useful for the preparation of these compositions are both well-known in the art and commercially available and include synthetic culture media. An exemplary synthetic medium is Dulbecco's minimal essential medium (DMEM; Dulbecco et al., Virol. 8, 396 (1959)) supplemented with 4.5 gm/l glucose, 0-20 mM glutamine, 0-20% fetal calf serum, several ppm amount of heavy metals, such as Cu, Mn, Fe, or Zn, etc., or/and the other heavy metals added in their salt forms, and with an anti-foaming agent, such as polyoxyethylene-polyoxypropylene block copolymer.

In addition, antibody-producing cell lines can also be created by techniques other than fusion, such as direct transformation of B lymphocytes with oncogenic DNA, or transfection with an oncovirus, such as Epstein-Barr virus (EBV, also called human herpesvirus 4 (HHV-4)) or Kaposi's sarcoma-associated herpesvirus (KSHV). See, U.S. Pat. Nos. 4,341,761; 4,399,121; 4,427,783; 4,444,887; 4,451,570; 4,466,917; 4,472,500; 4,491,632; 4,493,890. A monoclonal antibody may also be produced via an anti-receptor peptide or peptides containing the carboxyl terminal as described well-known in the art. See Niman et al., Proc. Natl. Acad. Sci. USA, 80: 4949-53 (1983); Geysen et al., Proc. Natl. Acad. Sci. USA, 82: 178-82 (1985); Lei et al. Biochemistry 34(20): 6675-88, (1995). Typically, the anti-receptor peptide or a peptide analog is used either alone or conjugated to an immunogenic carrier, as the immunogen for producing anti-receptor peptide monoclonal antibodies.

There are also a number of other well-known techniques for making monoclonal antibodies as binding molecules in this invention. Particularly useful are methods of making fully human antibodies. One method is phage display technology which can be used to select a range of human antibodies binding specifically to the antigen using methods of affinity enrichment. Phage display has been thoroughly described in the literature and the construction and screening of phage display libraries are well known in the art, see, e.g., Dente et al, Gene. 148(1):7-13 (1994); Little et al, Biotechnol Adv. 12(3): 539-55 (1994); Clackson et al., Nature 352: 264-8 (1991); Huse et al., Science 246: 1275-81 (1989).

Monoclonal antibodies derived by hybridoma technique from another species than human, such as mouse, can be humanized to avoid human anti-mouse antibodies when infused into humans. Among the more common methods of humanization of antibodies are complementarity-determining region grafting and resurfacing. These methods have been extensively described, see e.g. U.S. Pat. Nos. 5,859,205 and 6,797,492; Liu et al, Immunol Rev. 222: 9-27 (2008); Almagro et al, Front Biosci. 13: 1619-33 (2008); Lazar et al, Mol Immunol. 44(8): 1986-98 (2007); Li et al, Proc. Natl. Acad. Sci. USA. 103(10): 3557-62 (2006) each incorporated herein by reference. Fully human antibodies can also be prepared by immunizing transgenic mice, rabbits, monkeys, or other mammals, carrying large portions of the human immunoglobulin heavy and light chains, with an immunogen. Examples of such mice are: the Xenomouse. (Abgenix/Amgen), the HuMAb-Mouse (Medarex/BMS), the VelociMouse (Regeneron), see also U.S. Pat. Nos. 6,596,541, 6,207,418, 6,150,584, 6,111,166, 6,075,181, 5,922,545, 5,661,016, 5,545,806, 5,436,149 and 5,569,825. In human therapy, murine variable regions and human constant regions can also be fused to construct called “chimeric antibodies” that are considerably less immunogenic in man than murine mAbs (Kipriyanov et al, Mol Biotechnol. 26: 39-60 (2004); Houdebine, Curr Opin Biotechnol. 13: 625-9 (2002) each incorporated herein by reference). In addition, site-directed mutagenesis in the variable region of an antibody can result in an antibody with higher affinity and specificity for its antigen (Brannigan et al, Nat Rev Mol Cell Biol. 3: 964-70, (2002)); Adams et al, J Immunol Methods. 231: 249-60 (1999)) and exchanging constant regions of a mAb can improve its ability to mediate effector functions of binding and cytotoxicity.

Antibodies immunospecific for a malignant cell antigen can also be obtained commercially or produced by any method known to one of skill in the art such as, e.g., chemical synthesis or recombinant expression techniques. The nucleotide sequence encoding antibodies immune-specific for a malignant cell antigen can be obtained commercially, e.g., from the GenBank database or a database like it, the literature publications, or by routine cloning and sequencing.

Apart from an antibody, a peptide or protein that bind/block/target or in some other way interact with the epitopes or corresponding receptors on a targeted cell can be used as a binding molecule. These peptides or proteins could be any random peptide or proteins that have an affinity for the epitopes or corresponding receptors and they don't necessarily have to be of the immune-globulin family. These peptides can be isolated by similar techniques as for phage display antibodies (Szardenings, J Recept Signal Transduct Res. 2003, 23(4): 307-49). The use of peptides from such random peptide libraries can be similar to antibodies and antibody fragments. The binding molecules of peptides or proteins may be conjugated on or linked to a large molecules or materials, such as, but is not limited, an albumin, a polymer, a liposome, a nano particle, a dendrimer, as long as such attachment permits the peptide or protein to retain its antigen binding specificity.

Examples of antibodies used for conjugation of drugs via the linkers of this prevention for treating cancer, autoimmune disease, and/or infectious disease include, but are not limited to, 3F8 (anti-GD2), Abagovomab (anti CA-125), Abciximab (anti CD41 (integrin alpha-IIb), Adalimumab (anti-TNF-α), Adecatumumab (anti-EpCAM, CD326), Afelimomab (anti-TNF-α); Afutuzumab (anti-CD20), Alacizumab pegol (anti-VEGFR2), ALD518 (anti-IL-6), Alemtuzumab (Campath, MabCampath, anti-CD52), Altumomab (anti-CEA), Anatumomab (anti-TAG-72), Anrukinzumab (IMA-638, anti-IL-13), Apolizumab (anti-HLA-DR), Arcitumomab (anti-CEA), Aselizumab (anti-L-selectin (CD62L), Atlizumab (tocilizumab, Actemra, RoActemra, anti-IL-6 receptor), Atorolimumab (anti-Rhesus factor), Bapineuzumab (anti-beta amyloid), Basiliximab (Simulect, antiCD25 (a chain of IL-2 receptor), Bavituximab (anti-phosphatidylserine), Bectumomab (LymphoScan, anti-CD22), Belimumab (Benlysta, LymphoStat-B, anti-BAFF), Benralizumab (anti-CD125), Bertilimumab (anti-CCL11 (eotaxin-1)), Besilesomab (Scintimun, anti-CEA-related antigen), Bevacizumab (Avastin, anti-VEGF-A), Biciromab (FibriScint, anti-fibrin II beta chain), Bivatuzumab (anti-CD44 v6), Blinatumomab (BiTE, anti-CD19), Brentuximab (cAC10, anti-CD30 TNFRSF8), Briakinumab (anti-IL-12, IL-23) Canakinumab (Ilaris, anti-IL-1), Cantuzumab (C242, anti-CanAg), Capromab, Catumaxomab (Removab, anti-EpCAM, anti-CD3), CC49 (anti-TAG-72), Cedelizumab (anti-CD4), Certolizumab pegol (Cimzia anti-TNF-α), Cetuximab (Erbitux, IMC-C225, anti-EGFR), Citatuzumab bogatox (anti-EpCAM), Cixutumumab (anti-IGF-1), Clenoliximab (anti-CD4), Clivatuzumab (anti-MUC1), Conatumumab (anti-TRAIL-R2), CR6261 (anti-Influenza A hemagglutinin), Dacetuzumab (anti-CD40), Daclizumab (Zenapax, anti-CD25 (a chain of IL-2 receptor)), Daratumumab (anti-CD38 (cyclic ADP ribose hydrolase), Denosumab (Prolia, anti-RANKL), Detumomab (anti-B-lymphoma cell), Dorlimomab, Dorlixizumab, Ecromeximab (anti-GD3 ganglioside), Eculizumab (Soliris, anti-C5), Edobacomab (anti-endotoxin), Edrecolomab (Panorex, MAb17-1A, anti-EpCAM), Efalizumab (Raptiva, anti-LFA-1 (CD11a), Efungumab (Mycograb, anti-Hsp90), Elotuzumab (anti-SLAMF7), Elsilimomab (anti-IL-6), Enlimomab pegol (anti-ICAM-1 (CD54)), Epitumomab (anti-episialin), Epratuzumab (anti-CD22), Erlizumab (anti-ITGB2 (CD18)), Ertumaxomab (Rexomun, anti-HER2/neu, CD3), Etaracizumab (Abegrin, anti-integrin αᵥβ₃), Exbivirumab (anti-hepatitis B surface antigen), Fanolesomab (NeutroSpec, anti-CD15), Faralimomab (anti-interferon receptor), Farletuzumab (anti-folate receptor 1), Felvizumab (anti-respiratory syncytial virus), Fezakinumab (anti-IL-22), Figitumumab (anti-IGF-1 receptor), Fontolizumab (anti-IFN-γ), Foravirumab (anti-rabies virus glycoprotein), Fresolimumab (anti-TGF-β), Galiximab (anti-CD80), Gantenerumab (anti-beta amyloid), Gavilimomab (anti-CD147 (basigin)), Gemtuzumab (anti-CD33), Girentuximab (anti-carbonic anhydrase 9), Glembatumumab (CR011, anti-GPNMB), Golimumab (Simponi, anti-TNF-α), Gomiliximab (anti-CD23 (IgE receptor)), Ibalizumab (anti-CD4), Ibritumomab (anti-CD20), Igovomab (Indimacis-125, anti-CA-125), Imciromab (Myoscint, anti-cardiac myosin), Infliximab (Remicade, anti-TNF-α), Intetumumab (anti-CD51), Inolimomab (anti-CD25 (a chain of IL-2 receptor)), Inotuzumab (anti-CD22), Ipilimumab (anti-CD152), Iratumumab (anti-CD30 (TNFRSF8)), Keliximab (anti-CD4), Labetuzumab (CEA-Cide, anti-CEA), Lebrikizumab (anti-IL-13), Lemalesomab (anti-NCA-90 (granulocyte antigen)), Lerdelimumab (anti-TGF beta 2), Lexatumumab (anti-TRAIL-R2), Libivirumab (anti-hepatitis B surface antigen), Lintuzumab (anti-CD33), Lucatumumab (anti-CD40), Lumiliximab (anti-CD23 (IgE receptor), Mapatumumab (anti-TRAIL-R1), Maslimomab (anti-T-cell receptor), Matuzumab (anti-EGFR), Mepolizumab (Bosatria, anti-IL-5), Metelimumab (anti-TGF beta 1), Milatuzumab (anti-CD74), Minretumomab (anti-TAG-72), Mitumomab (BEC-2, anti-GD3 ganglioside), Morolimumab (anti-Rhesus factor), Motavizumab (Numax, anti-respiratory syncytial virus), Muromonab-CD3 (Orthoclone OKT3, anti-CD3), Nacolomab (anti-C242), Naptumomab (anti-5T4), Natalizumab (Tysabri, anti-integrin α₄), Nebacumab (anti-endotoxin), Necitumumab (anti-EGFR), Nerelimomab (anti-TNF-α), Nimotuzumab (Theracim, Theraloc, anti-EGFR), Nofetumomab, Ocrelizumab (anti-CD20), Odulimomab (Afolimomab, anti-LFA-1 (CD11a)), Ofatumumab (Arzerra, anti-CD20), Olaratumab (anti-PDGF-R α), Omalizumab (Xolair, anti-IgE Fc region), Oportuzumab (anti-EpCAM), Oregovomab (OvaRex, anti-CA-125), Otelixizumab (anti-CD3), Pagibaximab (anti-lipoteichoic acid), Palivizumab (Synagis, Abbosynagis, anti-respiratory syncytial virus), Panitumumab (Vectibix, ABX-EGF, anti-EGFR), Panobacumab (anti-Pseudomonas aeruginosa), Pascolizumab (anti-IL-4), Pemtumomab (Theragyn, anti-MUC1), Pertuzumab (Omnitarg, 2C4, anti-HER2/neu), Pexelizumab (anti-C5), Pintumomab (anti-adenocarcinoma antigen), Priliximab (anti-CD4), Pritumumab (anti-vimentin), PRO 140 (anti-CCR5), Racotumomab (1E10, anti-(N-glycolylneuraminic acid (NeuGc, NGNA)-gangliosides GM3)), Rafivirumab (anti-rabies virus glycoprotein), Ramucirumab (anti-VEGFR2), Ranibizumab (Lucentis, anti-VEGF-A), Raxibacumab (anti-anthrax toxin, protective antigen), Regavirumab (anti-cytomegalovirus glycoprotein B), Reslizumab (anti-IL-5), Rilotumumab (anti-HGF), Rituximab (MabThera, Rituxanmab, anti-CD20), Robatumumab (anti-IGF-1 receptor), Rontalizumab (anti-IFN-α), Rovelizumab (LeukArrest, anti-CD11, CD18), Ruplizumab (Antova, anti-CD154 (CD40L)), Satumomab (anti-TAG-72), Sevirumab (anti-cytomegalovirus), Sibrotuzumab (anti-FAP), Sifalimumab (anti-IFN-α), Siltuximab (anti-IL-6), Siplizumab (anti-CD2), (Smart) MI95 (anti-CD33), Solanezumab (anti-beta amyloid), Sonepcizumab (anti-sphingosine-1-phosphate), Sontuzumab (anti-episialin), Stamulumab (anti-myostatin), Sulesomab (LeukoScan, (anti-NCA-90 (granulocyte antigen), Tacatuzumab (anti-alpha-fetoprotein), Tadocizumab (anti-integrin α_{IIb}β₃), Talizumab (anti-IgE), Tanezumab (anti-NGF), Taplitumomab (anti-CD19), Tefibazumab (Aurexis, (anti-clumping factor A), Telimomab, Tenatumomab (anti-tenascin C), Teneliximab (anti-CD40), Teplizumab (anti-CD3), TGN1412 (anti-CD28), Ticilimumab (Tremelimumab, (anti-CTLA-4), Tigatuzumab (anti-TRAIL-R2), TNX-650 (anti-IL-13), Tocilizumab (Atlizumab, Actemra, RoActemra, (anti-IL-6 receptor), Toralizumab (anti-CD154 (CD40L)), Tositumomab (anti-CD20), Trastuzumab (Herceptin, (anti-HER2/neu), Tremelimumab (anti-CTLA-4), Tucotuzumab celmoleukin (anti-EpCAM), Tuvirumab (anti-hepatitis B virus), Urtoxazumab (anti-Escherichia coli), Ustekinumab (Stelara, anti-IL-12, IL-23), Vapaliximab (anti-AOC3 (VAP-1)), Vedolizumab, (anti-integrin α₄β₇), Veltuzumab (anti-CD20), Vepalimomab (anti-AOC3 (VAP-1), Visilizumab (Nuvion, anti-CD3), Vitaxin (anti-vascular integrin αvb3), Volociximab (anti-integrin α₅β₁), Votumumab (HumaSPECT, anti-tumor antigen CTAA16.88), Zalutumumab (HuMax-EGFr, (anti-EGFR), Zanolimumab (HuMax-CD4, anti-CD4), Ziralimumab (anti-CD147 (basigin)), Zolimomab (anti-CD5), Etanercept (Enbrel®), Alefacept (Amevive®), Abatacept (Orencia®), Rilonacept (Arcalyst), 14F7 [anti-IRP-2 (Iron Regulatory Protein 2)], 14G2a (anti-GD2 ganglioside, from Nat. Cancer Inst. for melanoma and solid tumors), J591 (anti-PSMA, Weill Cornell Medical School for prostate cancers), 225.28S [anti-HMW-MAA (High molecular weight-melanoma-associated antigen), Sorin Radiofarmaci S.R.L. (Milan, Italy) for melanoma], COL-1 (anti-CEACAM3, CGM1, from Nat. Cancer Inst. USA for colorectal and gastric cancers), CYT-356 (Oncoltad®, for prostate cancers), HNK20 (OraVax Inc. for respiratory syncytial virus), ImmuRAIT (from Immunomedics for NHL), Lym-1 (anti-HLA-DR10, Peregrine Pharm. for Cancers), MAK-195F [anti-TNF (tumor necrosis factor; TNFA, TNF-alpha; TNFSF2), from Abbott/Knoll for Sepsis toxic shock], MEDI-500 [T10B9, anti-CD3, TRαβ (T cell receptor alpha/beta), complex, from MedImmune Inc for Graft-versus-host disease], RING SCAN [anti-TAG 72 (tumour associated glycoprotein 72), from Neoprobe Corp. for Breast, Colon and Rectal cancers], Avicidin (anti-EPCAM (epithelial cell adhesion molecule), anti-TACSTD1 (Tumor-associated calcium signal transducer 1), anti-GA733-2 (gastrointestinal tumor-associated protein 2), anti-EGP-2 (epithelial glycoprotein 2); anti-KSA; KS1/4 antigen; M4S; tumor antigen 17-1A; CD326, from NeoRx Corp. for Colon, Ovarian, Prostate cancers and NHL]; LymphoCide (Immunomedics, NJ), Smart ID10 (Protein Design Labs), Oncolym (Techniclone Inc, CA), Allomune (BioTransplant, CA), anti-VEGF (Genentech, CA); CEAcide (Immunomedics, NJ), IMNC-1C11 (ImClone, NJ) and Cetuximab (ImClone, NJ).

Other antibodies as cell binding molecules/ligands include, but are not limited to, are antibodies against the following antigens: Aminopeptidase N (CD13), Annexin A1, B7-H3 (CD276, various cancers), CA125 (ovarian), CA15-3 (carcinomas), CA19-9 (carcinomas), L6 (carcinomas), Lewis Y (carcinomas), Lewis X (carcinomas), alpha fetoprotein (carcinomas), CA242 (colorectal), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (Metastatic melanoma, sarcoma), CD52 (leukemia), CD56 (small cell lung cancers, ovarian cancer, Merkel cell carcinoma, and the liquid tumor, multiple myeloma), CD66e (cancers), CD70 (metastatic renal cell carcinoma and non-Hodgkin lymphoma), CD74 (multiple myeloma), CD80 (lymphoma), CD98 (cancers), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (carcinoembryonic antigen; CEA, CD66e) (breast, colorectal and lung cancers), DLL3 (delta-like-3), DLL4 (delta-like-4), EGFR (Epidermal Growth Factor Receptor, various cancers), CTLA4 (melanoma), CXCR4 (CD184, Heme-oncology, solid tumors), Endoglin (CD105, solid tumors), EPCAM (epithelial cell adhesion molecule, bladder, head, neck, colon, NHL prostate, and ovarian cancers), ERBB2 (Epidermal Growth Factor Receptor 2; lung, breast, prostate cancers), FCGR1 (autoimmune diseases), FOLR (folate receptor, ovarian cancers), GD2 ganglioside (cancers), G-28 (a cell surface antigen glyvolipid, melanoma), GD3 idiotype (cancers), Heat shock proteins (cancers), HER1 (lung, stomach cancers), HER2 (breast, lung and ovarian cancers), HLA-DR10 (NHL), HLA-DRB (NHL, B cell leukemia), human chorionic gonadotropin (carcinoma), IGF1R (insulin-like growth factor 1 receptor, solid tumors, blood cancers), IL-2 receptor (interleukin 2 receptor, T-cell leukemia and lymphomas), IL-6R (interleukin 6 receptor, multiple myeloma, RA, Castleman's disease, IL6 dependent tumors), Integrins (αvβ3, α5β1, α6β4, αllβ3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 or MUC1-KLH (breast, ovarian, cervix, bronchus and gastrointestinal cancer), MUC16 (CA125) (Ovarian cancers), CEA (colorectal), gp100 (melanoma), MART1 (melanoma), MPG (melanoma), MS4A1 (membrane-spanning 4-domains subfamily A, small cell lung cancers, NHL), Nucleolin, Neu oncogene product (carcinomas), P21 (carcinomas), Paratope of anti-(N-glycolylneuraminic acid, Breast, Melanoma cancers), PLAP-like testicular alkaline phosphatase (ovarian, testicular cancers), PSMA (prostate tumors), PSA (prostate), ROBO4, TAG 72 (tumour associated glycoprotein 72, AML, gastric, colorectal, ovarian cancers), T cell transmembrane protein (cancers), Tie (CD202b), TNFRSF10B (tumor necrosis factor receptor superfamily member 10B, cancers), TNFRSF13B (tumor necrosis factor receptor superfamily member 13B, multiple myeloma, NHL, other cancers, RA and SLE), TPBG (trophoblast glycoprotein, Renal cell carcinoma), TRAIL-R1 (Tumor necrosis apoprosis Inducing ligand Receptor 1, lymphoma, NHL, colorectal, lung cancers), VCAM-1 (CD106, Melanoma), VEGF, VEGF-A, VEGF-2 (CD309) (various cancers). Some other tumor associated antigens recognized by antibodies have been reviewed (Gerber, et al, mAbs 1:3, 247-53 (2009); Novellino et al, Cancer Immunol Immunother. 54(3), 187-207 (2005). Franke, et al, Cancer Biother Radiopharm. 2000, 15, 459-76).

The cell-binding agents, more preferred antibodies, can be any agents that are able to against tumor cells, virus infected cells, microorganism infected cells, parasite infected cells, autoimmune cells, activated cells, myeloid cells, activated T-cells, B cells, or melanocytes. More specifically the cell binding agents can be any agent/molecule that is able to against any one of the following antigens or receptors: CD2, CD2R, CD3, CD3gd, CD3e, CD4, CD5, CD6, CD7, CD8, CD8a, CD8b, CD9, CD10, CD11a, CD11b, CD11c, CD12, CD12w, CD13, CD14, CD15, CD15s, CD15u, CD16, CD16a, CD16b, CD17, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD42a, CD42b, CD42c, CD42d, CD43, CD44, CD44R, CD45, CD45RA, CD45RB, CD45RO, CD46, CD47, CD47R, CD48, CD49a, CD49b, CD49c, CD49e, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD74, CD75, CD75s, CD76, CD77, CD78, CD79, CD79a, CD79b, CD80, CD81, CD82, CD83, CD84, CDw84, CD85, CD86, CD87, CD88, CD89, CD90, CD91, CD92, CDw92, CD93, CD94, CD95, CD96, CD97, CD98, CD99, CD99R, CD100, CD101, CD102, CD103, CD104, CD105, CD106, CD107, CD107a, CD107b, CD108, CD109, CD110, CD111, CD112, CD113, CDw113, CD114, CD115, CD116, CD117, CD118, CD119, CDw119, CD120a, CD120b, CD121a, CD121b, CDw121b, CD122, CD123, CDw123, CD124, CD125, CDw125, CD126, CD127, CD128, CDw128, CD129, CD130, CD131, CDw131, CD132, CD133, CD134, CD135, CD136, CDw136, CD137, CDw137, CD138, CD139, CD140a, CD140b, CD141, CD142, CD143, CD144, CD145, CDw145, CD146, CD147, CD148, CD149, CD150, CD151, CD152, CD153, CD154, CD155, CD156a, CD156b, CDw156c, CD157, CD158a, CD158b, CD159a, CD159b, CD159c, CD160, CD161, CD162, CD162R, CD163, CD164, CD165, CD166, CD167, CD167a, CD168, CD169, CD170, CD171, CD172a, CD172b, CD172g, CD173, CD174, CD175, CD175s, CD176, CD177, CD178, CD179, CD180, CD181, CD182, CD183, CD184, CD185, CD186, CDw186, CD187, CD188, CD189, CD190, Cd191, CD192, CD193, CD194, CD195, CD196, CD197, CD198, CDw198, CD199, CDw199, CD200, CD200a, CD200b, CD201, CD202, CD202b, CD203, CD203c, CD204, CD205, CD206, CD207, CD208, CD209, CD210, CDw210, CD211, CD212, CD213a1, CD213a2, CD214, CD215, CD216, CDw217, CDw218a, CDw218b, CD219, CD220, CD221, CD222, CD223, CD224, CD225, CD226, CD227, CD228, CD229, CD230, CD231, CD232, CD233, CD234, CD235a, CD235ab, CD235b, CD236, CD236R, CD237, CD238, CD239, CD240, CD240CE, CD240D, CD241, CD242, CD243, CD244, CD245, CD246, CD247, CD248, CD249, CD250, CD251, CD252, CD253, CD254, CD256, CD257, CD258, CD259, CD260, CD261, CD262, CD263, CD264, CD265, CD266, CD267, CD268, CD269, CD270, CD271, CD272, CD273, CD274, CD275, CD276 (B7-H3), CD277, CD278, CD279, CD280, CD281, CD282, CD283, CD284, CD285, CD286, CD287, CD288, CD289, CD290, CD291, CD292, CDw293, CD294, CD295, CD296, CD297, CD298, CD299, CD300a, CD300c, CD300e, CD301, CD302, CD303, CD304, CD305, CD306, CD307, CD308, CD309, CD310, CD311, CD312, CD314, CD315, CD316, CD317, CD318, CD319, CD320, CD321, CD322, CD323, CD324, CDw325, CD326, CDw327, CDw328, CDw329, CD330, CD331, CD332, CD333, CD334, CD335, CD336, CD337, CDw338, CD339, 4-1BB, 5AC, 5T4 (Trophoblast glycoprotein, TPBG, 5T4, Wnt-Activated Inhibitory Factor 1 or WAIF1), Adenocarcinomaantigen, AGS-5, AGS-22M6, Activin receptor-like kinase 1, AFP, AKAP-4, ALK, Alpha intergrin, Alpha v beta6, Amino-peptidase N, Amyloid beta, Androgen receptor, Angiopoietin 2, Angiopoietin 3, Annexin A1, Anthrax toxin-protective antigen, Anti-transferrin receptor, AOC3 (VAP-1), B7-H3, Bacillus anthracisanthrax, BAFF (B-cell activating factor), B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11 (C—C motif chemokine 11), CCR4 (C—C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD115), CSF2 (colony stimulating factor 2, Granulocyte-macrophage colony-stimulating factor (GM-CSF)), CTLA4 (cytotoxic T-lymphocyte associated protein 4), CTAA16.88 tumor antigen, CXCR4 (CD184), C—X—C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxintype-1, E. coli shiga toxintype-2, ED-B, EGFL7 (EGF-like domain-containing protein 7), EGFR, EGFRII, EGFRvIII, Endoglin (CD105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation proteinalpha), FCGR1, alpha-Fetoprotein, Fibrin II, beta chain, Fibronectin extra domain-B, FOLR (folate receptor), Folate receptor alpha, Folate hydrolase, Fos-related antigen 1, F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influeza hemag-glutinin, IgE, IgE Fc region, IGHE, interleukins (e.g. IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (a4, α_{IIb}β₃, αvβ3, α₄β₇, α5β1, α6β4, α7β7, αllβ3, α5β5, αvβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (mesothelin), MUC1 (Mucin 1, cell surface associated (MUC1) orpolymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RANTES receptors (CCR1, CCR3, CCR5), RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R₁ (Tumor necrosis apoprosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TROP-2, TRP-2, Tyrosinase, VCAM-1 (CD106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

In another specific embodiment, the cell-binding ligand-drug conjugates via the bridge linkers of this invention are used for the targeted treatment of cancers. The targeted cancers include, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metasatic Squamous Neck Cancer with Occult Primary, Multiple Myeloma, and Other Plasma Cell Neoplasms, Mycosis Fungoides, Myelodysplastic Syndrome, Myeloproli-ferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor.

In another specific embodiment, the cell-binding-drug conjugates of this invention are used in accordance with the compositions and methods for the treatment or prevention of an autoimmune disease. The autoimmune diseases include, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaff s encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barre syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogamma-globulinemia, Idiopathic Inflammatory Demyelinating Diseases, Idiopathic pulmonary fibrosis, Idiopathic thrombocytopenic purpura (See Autoimmune thrombocytopenic purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, Meniere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjögren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome

In another specific embodiment, a binding molecule used for the conjugate via the bis-linkers of this invention for the treatment or prevention of an autoimmune disease can be, but are not limited to, anti-elastin antibody; Abys against epithelial cells antibody; Anti-Basement Membrane Collagen Type IV Protein antibody; Anti-Nuclear Antibody; Anti ds DNA; Anti ss DNA, Anti Cardiolipin Antibody IgM, IgG; anti-celiac antibody; Anti Phospholipid Antibody IgK, IgG; Anti SM Antibody; Anti Mitochondrial Antibody; Thyroid Antibody; Microsomal Antibody, T-cells antibody; Thyroglobulin Antibody, Anti SCL-70; Anti-Jo; Anti-U.sub.IRNP; Anti-La/SSB; Anti SSA; Anti SSB; Anti Perital Cells Antibody; Anti Histones; Anti RNP; C-ANCA; P-ANCA; Anti centromere; Anti-Fibrillarin, and Anti GBM Antibody, Anti-ganglioside antibody; Anti-Desmogein 3 antibody; Anti-p62 antibody; Anti-sp100 antibody; Anti-Mitochondrial (M2) antibody; Rheumatoid factor antibody; Anti-MCV antibody; Anti-topoisomerase antibody; Anti-neutrophil cytoplasmic (cANCA) antibody.

In certain preferred embodiments, the binding molecule for the conjugate in the present invention, can bind to both a receptor and a receptor complex expressed on an activated lymphocyte which is associated with an autoimmune disease. The receptor or receptor complex can comprise an immunoglobulin gene superfamily member (e.g. CD2, CD3, CD4, CD8, CD19, CD20, CD22, CD25, CD27, CD28, CD30, CD33, CD37, CD38, CD56, CD70, CD79, CD79b, CD90, CD125, CD137, CD138, CD147, CD152/CTLA-4, PD-1, PD-L1, or ICOS), a TNF receptor superfamily member (e.g. CD27, CD40, CD95/Fas, CD134/OX40, CD137/4-1BB, INF-R1, TNFR-2, RANK, TACI, BCMA, osteoprotegerin, Apo2/TRAIL-R1, TRAIL-R2, TRAIL-R3, TRAIL-R4, and APO-3), an integrin, a cytokine receptor, a chemokine receptor, a major histocompatibility protein, a lectin (C-type, S-type, or I-type), or a complement control protein.

In another specific embodiment, useful cell binding ligands that are immunospecific for a viral or a microbial antigen are humanized or human monoclonal antibodies. As used herein, the term “viral antigen” includes, but is not limited to, any viral peptide, polypeptide protein (e.g. HIV gp120, HIV nef, RSV F glycoprotein, influenza virus neuramimi-dase, influenza virus hemagglutinin, HTLV tax, herpes simplex virus glycoprotein (e.g. gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., a bacteria, fungi, pathogenic protozoa, or yeast polypeptides including, e.g., LPS and capsular polysaccharide 5/8) that is capable of eliciting an immune response. Examples of antibodies available 1 for the viral or microbial infection include, but are not limited to, Palivizumab which is a humanized anti-respiratory syncytial virus monoclonal antibody for the treatment of RSV infection; PR0542 which is a CD4 fusion antibody for the treatment of HIV infection; Ostavir which is a human antibody for the treatment of hepatitis B virus; PROTVIR which is a humanized IgG.sub.1 antibody for the treatment of cytomegalovirus; and anti-LPS antibodies.

The cell binding molecules-drug conjugates via the bis-linkers of this invention can be used in the treatment of infectious diseases. These infectious diseases include, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcano-bacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioido-mycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome, Giardiasis, Glanders, Gnathosto-miasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsial-pox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuberculosis infection, Yersiniosis, Yellow fever, Zygomycosis.

The cell binding molecule, which is more preferred to be an antibody described in this patent that are against pathogenic strains include, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4), Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli O157.H7, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomy-xoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthora-mycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

Other antibodies as cell binding ligands used in this invention for treatment of viral disease include, but are not limited to, antibodies against antigens of pathogenic viruses, including as examples and not by limitation: Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytialvirus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the conjugate of the invention together with a pharmaceutically acceptable carrier, diluent, or excipient for treatment of cancers, infections or autoimmune disorders. The method for treatment of cancers, infections and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 30 minutes to about 48 hours at about 37° C. The exact conditions of concentration and time of incubation (=dose) are readily determined by the skilled clinicians. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

Drugs/Cytotoxic Agents for Conjugation

Drugs that can be conjugated to a cell-binding molecule in the present invention are small molecule drugs including cytotoxic agents, which can be linked to or after they are modified for linkage to the cell-binding agent. A “small molecule drug” is broadly used herein to refer to an organic, inorganic, or organometallic compound that may have a molecular weight of, for example, 100 to 4000, more suitably from 200 to 3000. Small molecule drugs are well characterized in the art, such as in WO05058367A2, and in U.S. Pat. No. 4,956,303, among others and are incorporated in their entirety by reference. The drugs include known drugs and those that may become known drugs.

Drugs that are known include, but not limited to,

1). Chemotherapeutic agents: a). Alkylating agents: such as Nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 (including its adozelesin, carzelesin and bizelesin synthetic analogues); Duocarmycin (including the synthetic analogues, KW-2189, CBI-TMI, and CBI dimers); Benzodiazepine dimers (e.g., dimers of pyrrolobenzodiazepine (PBD) or tomaymycin, indolinobenzodiazepines, imidazobenzothiadiazepines, or oxazolidinobenzodiazepines); Nitrosoureas: (carmustine, lomustine, chlorozotocin, fotemustine, nimustine, ranimustine); Alkylsulphonates: (busulfan, treosulfan, improsulfan and piposulfan); Triazenes: (dacarbazine); Platinum containing compounds: (carboplatin, cisplatin, oxaliplatin); aziridines, such as benzodopa, carboquone, meturedopa, and uredopa; ethylenimines and methylamelamines including altretamine, triethylenemel-amine, trietylenephosphoramide, triethylenethio-phosphaoramide and trimethylolomel-amine]; b). Plant Alkaloids: such as Vinca alkaloids: (vincristine, vinblastine, vindesine, vinorelbine, navelbin); Taxoids: (paclitaxel, docetaxol) and their analogs, Maytansinoids (DM1, DM2, DM3, DM4, maytansine and ansamitocins) and their analogs, cryptophycins (particularly cryptophycin 1 and cryptophycin 8); epothilones, eleutherobin, discodermolide, bryostatins, dolostatins, auristatins, tubulysins, cephalostatins; pancratistatin; erbulins; a sarcodictyin; spongistatin; c). DNA Topoisomerase Inhibitors: such as [Epipodophyllins: (9-aminocamptothecin, camptothecin, crisnatol, daunomycin, etoposide, etoposide phosphate, irinotecan, mitoxantrone, novantrone, retinoic acids (retinols), teniposide, topotecan, 9-nitrocamptothecin (RFS 2000)); mitomycins: (mitomycin C) and its analogs]; d). Anti-metabolites: such as {[Anti-folate: DHFR inhibitors: (methotrexate, trimetrexate, denopterin, pteropterin, aminopterin (4-aminopteroic acid) or the other folic acid analogues); IMP dehydrogenase Inhibitors: (mycophenolic acid, tiazofurin, ribavirin, EICAR); Ribonucleotide reductase Inhibitors: (hydroxyurea, deferoxamine)]; [Pyrimidine analogs: Uracil analogs: (ancitabine, azacitidine, 6-azauridine, capecitabine (Xeloda), carmofur, cytarabine, dideoxyuridine, doxifluridine, enocitabine, 5-Fluorouracil, floxuridine, ratitrexed (Tomudex)); Cytosine analogs: (cytarabine, cytosine arabinoside, fludarabine); Purine analogs: (azathioprine, fludarabine, mercaptopurine, thiamiprine, thioguanine)]; folic acid replenisher, such as frolinic acid}; and Inhibitors of nicotinamide phosphoribosyltransferase (NAMPT); e). Hormonal therapies: such as {Receptor antagonists: [Anti-estrogen: (megestrol, raloxifene, tamoxifen); LHRH agonists: (goscrclin, leuprolide acetate); Anti-androgens: (bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (CB 1093, EB 1089 KH 1060, cholecalciferol, ergocalciferol); Photodynamic therapies: (verteporfin, phthalocyanine, photosensitizer Pc4, demethoxyhypocrellin A); Cytokines: (Interferon-alpha, Interferon-gamma, tumor necrosis factor (TNFs), human proteins containing a TNF domain)]}; f). Kinase inhibitors, such as BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib; g). A poly (ADP-ribose) polymerase (PARP) inhibitors, such as olaparib, niraparib, iniparib, talazoparib, veliparib, veliparib, CEP 9722 (Cephalon's), E7016 (Eisai's), BGB-290 (BeiGene's), 3-aminobenzamide.

h). antibiotics, such as the enediyne antibiotics (e.g. calicheamicins, especially calicheamicin γ1, δ1, α1 and β1, see, e.g., J. Med. Chem., 39 (11), 2103-2117 (1996), Angew Chem Intl. Ed. Engl. 33:183-186 (1994); dynemicin, including dynemicin A and deoxydynemicin; esperamicin, kedarcidin, C-1027, maduropeptin, as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; i). Others: such as Polyketides (acetogenins), especially bullatacin and bullatacinone; gemcitabine, epoxomicins (e. g. carfilzomib), bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors (such as Lovastatin), Dopaminergic neurotoxins (such as 1-methyl-4-phenylpyridinium ion), Cell cycle inhibitors (such as staurosporine), Actinomycins (such as Actinomycin D, dactinomycin), Bleomycins (such as bleomycin A2, bleomycin B2, peplomycin), Anthracyclines (such as daunorubicin, doxorubicin (adriamycin), idarubicin, epirubicin, eribulin, pirarubicin, zorubicin, mtoxantrone, MDR inhibitors (such as verapamil), Ca²⁺ATPase inhibitors (such as thapsigargin), Histone deacetylase inhibitors (Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, such as aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; Polysaccharide-K (PSK©); razoxane; rhizoxin; sizofiran; spirogermanium; tenuazonic acid; triaziquone; 2,2′,2″-trichlorotriethylamine; trichothecenes (especially T-2 toxin, verrucarin A, roridin A and anguidine); urethane, siRNA, antisense drugs, and a nucleolytic enzyme.

2). An anti-autoimmune disease agent includes, but is not limited to, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.

3). An anti-infectious disease agent includes, but is not limited to, a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin; b). Amphenicols:azidamfenicol, chloramphenicol, florfenicol, thiamphenicol; c). Ansamycins: geldanamycin, herbimycin; d). Carbapenems: biapenem, doripenem, ertapenem, imipenem/cilastatin, meropenem, panipenem; e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef); f). Glycopeptides: bleomycin, vancomycin (oritavancin, telavancin), teicoplanin (dalbavancin), ramoplanin; g). Glycylcyclines: e. g. tigecycline; g). β-Lactamase inhibitors: penam (sulbactam, tazobactam), clavam (clavulanic acid); i). Lincosamides: clindamycin, lincomycin; j). Lipopeptides: daptomycin, A54145, calcium-dependent antibiotics (CDA); k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin; l). Monobactams: aztreonam, tigemonam; m). Oxazolidinones: linezolid; n). Penicillins: amoxicillin, ampicillin (pivampicillin, hetacillin, bacampicillin, metampicillin, talampicillin), azidocillin, azlocillin, benzylpenicillin, benzathine benzylpenicillin, benzathine phenoxymethyl-penicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin; o). Polypeptides: bacitracin, colistin, polymyxin B; p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin; q). Streptogramins: pristinamycin, quinupristin/dalfopristin); r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole); s). Steroid antibacterials: e.g. fusidic acid; t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (e.g. tigecycline); u). Other types of antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (e. g. fosfomycin), nitrofurantoin, paclitaxel, platensimycin, pyrazinamide, quinupristin/dalfopristin, rifampicin (rifampin), tazobactam tinidazole, uvaricin;

4). Anti-viral drugs: a). Entry/fusion inhibitors: aplaviroc, maraviroc, vicriviroc, gp41 (enfuvirtide), PRO 140, CD4 (ibalizumab); b). Integrase inhibitors: raltegravir, elvitegravir, globoidnan A; c). Maturation inhibitors: bevirimat, vivecon; d). Neuraminidase inhibitors: oseltamivir, zanamivir, peramivir; e). Nucleosides & nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′,3′-dideoxynucleoside analogues (e.g. 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (e.g. β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT); f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine; g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir; h). Other types of anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib. 5). The drugs used for conjugates via a bis-linker of the present invention also include radioisotopes. Examples of radioisotopes (radionuclides) are ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. Radioisotope labeled antibodies are useful in receptor targeted imaging experiments or can be for targeted treatment such as with the antibody-drug conjugates of the invention (Wu et al (2005) Nature Biotechnology 23(9): 1137-46). The cell binding molecules, e.g. an antibody can be labeled with ligand reagents through the bridge linkers of the present patent that bind, chelate or otherwise complex a radioisotope metal, using the techniques described in Current Protocols in Immunology, Volumes 1 and 2, Coligen et al, Ed. Wiley-Interscience, New York, Pubs. (1991). Chelating ligands which may complex a metal ion include DOTA, DOTP, DOTMA, DTPA and TETA (Macrocyclics, Dallas, Tex. USA).

6). The pharmaceutically acceptable salts, acids, derivatives, hydrate or hydrated salt; or a crystalline structure; or an optical isomer, racemate, diastereomer or enantiomer of any of the above drugs.

In another embodiment, the drug/cytotoxic molecule in the Formula (I) and/or (II) can be a chromophore molecule, for which the conjugate can be used for detection, monitoring, or study the interaction of the cell binding molecule with a target cell. Chromophore molecules are a compound that have the ability to absorb a kind of light, such as UV light, florescent light, IR light, near IR light, visual light; A chromatophore molecule includes a class or subclass of xanthophores, erythrophores, iridophores, leucophores, melanophores, and cyanophores; a class or subclass of fluorophore molecules which are fluorescent chemical compounds re-emitting light upon light; a class or subclass of visual phototransduction molecules; a class or subclass of photophore molecules; a class or subclass of luminescence molecules; and a class or subclass of luciferin compounds.

The chromophore molecule can be selected from, but not limited, non-protein organic fluorophores, such as: Xanthene derivatives (fluorescein, rhodamine, Oregon green, eosin, and Texas red); Cyanine derivatives: (cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (dansyl and prodan derivatives); Coumarin derivatives; Oxadiazole derivatives (pyridyloxazole, nitrobenzoxadiazole and benzoxadiazole); Anthracene derivatives (anthraquinones, including DRAQ5, DRAQ7 and CyTRAK Orange); Pyrene derivatives (cascade blue, etc.); Oxazine derivatives (Nile red, Nile blue, cresyl violet, oxazine 170 etc.). Acridine derivatives (proflavin, acridine orange, acridine yellow etc.). Arylmethine derivatives (auramine, crystal violet, malachite green). Tetrapyrrole derivatives (porphin, phthalocyanine, bilirubin).

Or a chromophore molecule can be selected from any analogs and derivatives of the following fluorophore compounds: CF dye (Biotium), DRAQ and CyTRAK probes (BioStatus), BODIPY (Invitrogen), Alexa Fluor (Invitrogen), DyLight Fluor (Thermo Scientific, Pierce), Atto and Tracy (Sigma Aldrich), FluoProbes (Interchim), Abberior Dyes (Abberior), DY and MegaStokes Dyes (Dyomics), Sulfo Cy dyes (Cyandye), HiLyte Fluor (AnaSpec), Seta, SeTau and Square Dyes (SETA BioMedicals), Quasar and Cal Fluor dyes (Biosearch Technologies), SureLight Dyes (APC, RPEPerCP, Phycobilisomes)(Columbia Biosciences), APC, APCXL, RPE, BPE (Phyco-Biotech).

Examples of the widely used fluorophore compounds which are reactive or conjugatable with the linkers of the invention are: Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, IR-783, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin (PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine.

The fluorophore compounds that can be linked to the linkers of the invention for study of nucleic acids or proteins are selected from the following compounds or their derivatives: 7-AAD (7-aminoactinomycin D, CG-selective), Acridine Orange, Chromomycin A3, CyTRAK Orange (Biostatus, red excitation dark), DAPI, DRAQ5, DRAQ7, Ethidium Bromide, Hoechst33258, Hoechst33342, LDS 751, Mithramycin, PropidiumIodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1. The fluorophore compounds that can be linked to the linkers of the invention for study cells are selected from the following compounds or their derivatives: DCFH (2′7′Dichorodihydro-fluorescein, oxidized form), DHR (Dihydrorhodamine 123, oxidized form, light catalyzes oxidation), Fluo-3 (AM ester. pH>6), Fluo-4 (AM ester. pH 7.2), Indo-1 (AM ester, low/high calcium (Ca2+)), and SNARF (pH 6/9). The preferred fluorophore compounds that can be linked to the linkers of the invention for study proteins/antibodies are selected from the following compounds or their derivatives: Allophycocyanin (APC), AmCyan1 (tetramer, Clontech), AsRed2 (tetramer, Clontech), Azami Green (monomer, MBL), Azurite, B-phycoerythrin (BPE), Cerulean, CyPet, DsRed monomer (Clontech), DsRed2 (“RFP”, Clontech), EBFP, EBFP2, ECFP, EGFP (weak dimer, Clontech), Emerald (weak dimer, Invitrogen), EYFP (weak dimer, Clontech), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRed1, J-Red, Katusha, Kusabira Orange (monomer, MBL), mCFP, mCherry, mCitrine, Midoriishi Cyan (dimer, MBL), mKate (TagFP635, monomer, Evrogen), mKeima-Red (monomer, MBL), mKO, mOrange, mPlum, mRaspberry, mRFP1 (monomer, Tsien lab), mStrawberry, mTFP1, mTurquoise2, P3 (phycobilisome complex), Peridinin Chlorophyll (PerCP), R-phycoerythrin (RPE), T-Sapphire, TagCFP (dimer, Evrogen), TagGFP (dimer, Evrogen), TagRFP (dimer, Evrogen), TagYFP (dimer, Evrogen), tdTomato (tandem dimer), Topaz, TurboFP602 (dimer, Evrogen), TurboFP635 (dimer, Evrogen), TurboGFP (dimer, Evrogen), TurboRFP (dimer, Evrogen), TurboYFP (dimer, Evrogen), Venus, Wild Type GFP, YPet, ZsGreen1 (tetramer, Clontech), ZsYellow1 (tetramer, Clontech).

The examples of the structure of the conjugates of the antibody-chromophore molecules via the bridge linker are as following Ac01, Ac02, Ac03, Ac04, Ac05, Ac06, and Ac07, Ac08, Ac09, Ac010, and Ac11:

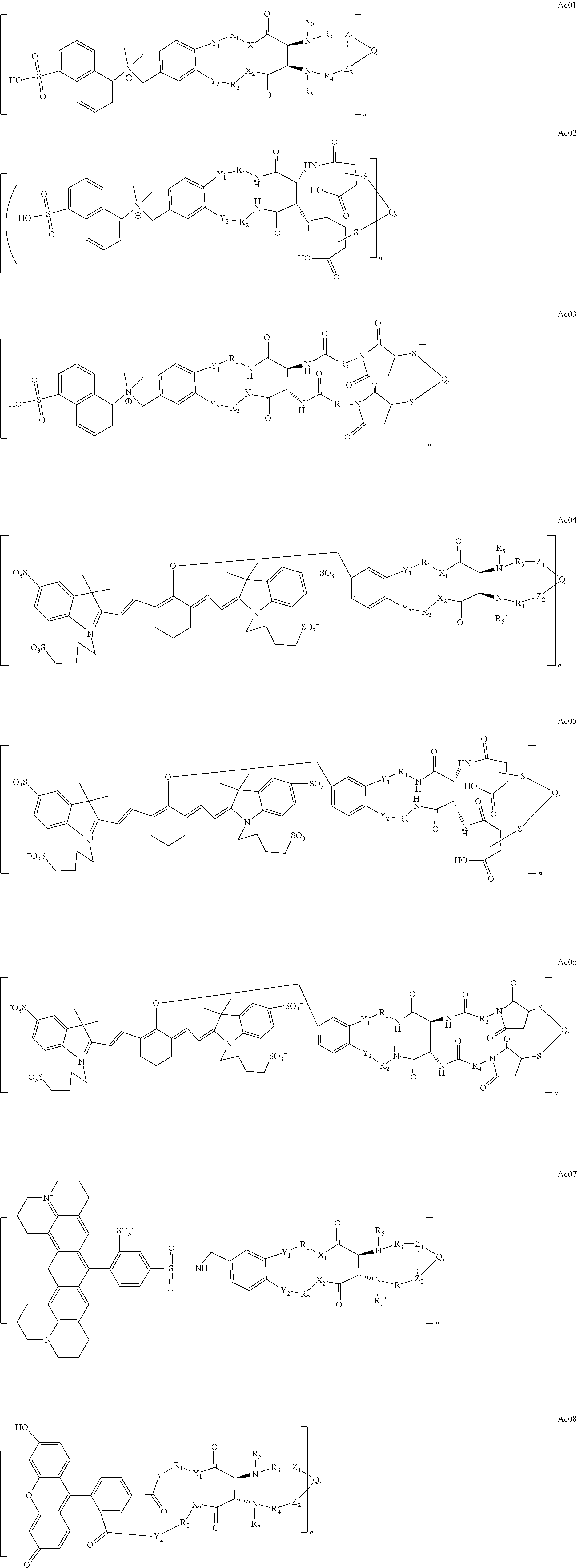

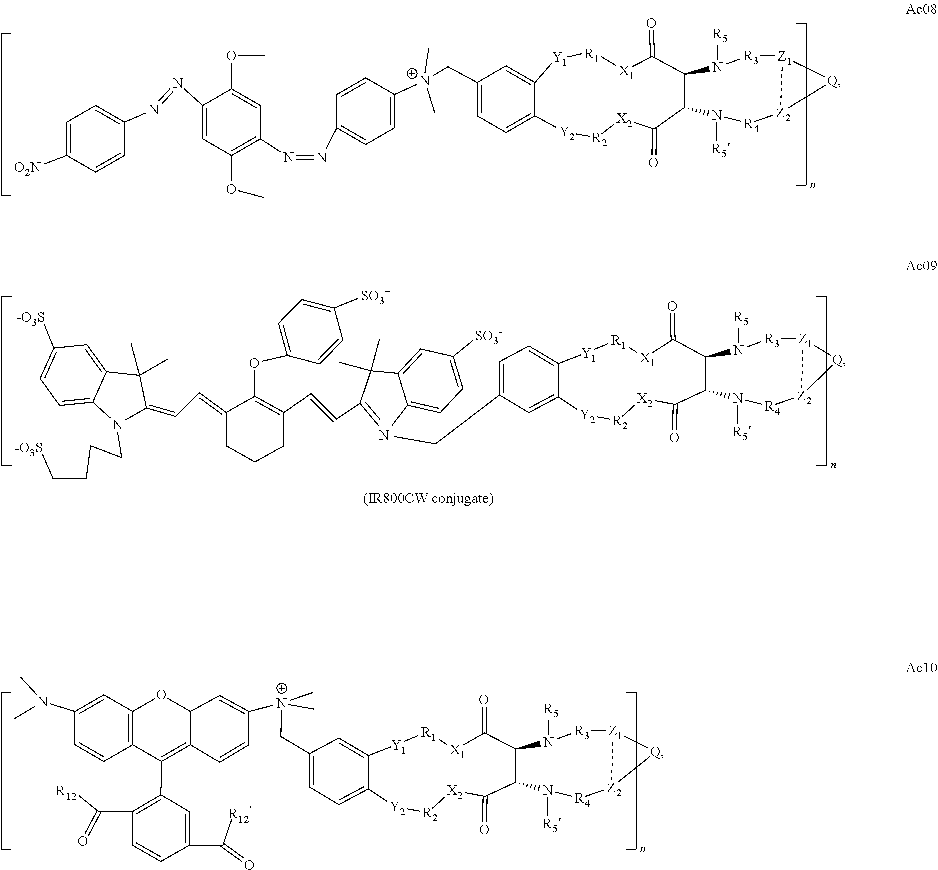

wherein “-----”, Q, Y₁, Y₂, R₁, R₂, R₃, R₄, R₅, R_{5′}, Z₁, Z₂, and n are defined the same above; R₁₂ and R_{12′} are independently OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, O(CH₂CH₂O)ₚCH₂CH₂COOH, NH(CH₂CH₂O)ₚCH₂CH₂COOH, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, R₁—NHPO₃H₂, R₁—OPO₃H₂, O(CH₂CH₂O)ₚCH₂CH₂OPO₃H₂, NH(CH₂CH₂O)ₚCH₂CH₂NHPO₃H₂, OR₁—NHPO₃H₂, NH—R₁—NHPO₃H₂, NH—Ar—COOH, NH—Ar—NH₂, wherein p=0-5000, Aa is an aminoacid, (Aa)ₙ comprises the same or different, natural or unnatural amino acids, n=1-30.

In another embodiment, the drug in the Formula (I), (II), (III) and (IV) can be polyalkylene glycols that are used for extending the half-life of the cell-binding molecule when administered to a mammal. Polyalkylene glycols include, but are not limited to, poly(ethylene glycols) (PEGs), poly(propylene glycol) and copolymers of ethylene oxide and propylene oxide; particularly preferred are PEGs, and more particularly preferred are monofunctionally activated hydroxyPEGs (e.g., hydroxyl PEGs activated at a single terminus, including reactive esters of hydroxyPEG-monocarboxylic acids, hydroxyPEG-monoaldehydes, hydroxyPEG-monoamines, hydroxyPEG-monohydrazides, hydroxyPEG-monocarbazates, hydroxyl PEG-monoiodo-acetamides, hydroxyl PEG-monomaleimides, hydroxyl PEG-monoorthopyridyl disulfides, hydroxyPEG-monooximes, hydroxyPEG-monophenyl carbonates, hydroxyl PEG-monophenyl glyoxals, hydroxyl PEG-monothiazolidine-2-thiones, hydroxyl PEG-monothioesters, hydroxyl PEG-monothiols, hydroxyl PEG-monotriazines and hydroxyl PEG-monovinylsulfones).

In certain such embodiments, the polyalkylene glycol has a molecular weight of from about 10 Daltons to about 200 kDa, preferably about 88 Da to about 50 kDa; two branches each with a molecular weight of about 88 Da to about 50 kDa; and more preferably two branches, each of about 88 Da to about 20 kDa. In one particular embodiment, the polyalkylene glycol is poly(ethylene) glycol and has a molecular weight of about 10 kDa; about 20 kDa, or about 40 kDa. In specific embodiments, the PEG is a PEG 10 kDa (linear or branched), a PEG 20 kDa (linear or branched), or a PEG 40 kDa (linear or branched). A number of US patents have disclosed the preparation of linear or branched “non-antigenic” PEG polymers and derivatives or conjugates thereof, e.g., U.S. Pat. Nos. 5,428,128; 5,621,039; 5,622,986; 5,643,575; 5,728,560; 5,730,990; 5,738,846; 5,811,076; 5,824,701; 5,840,900; 5,880,131; 5,900,402; 5,902,588; 5,919,455; 5,951,974; 5,965,119; 5,965,566; 5,969,040; 5,981,709; 6,011,042; 6,042,822; 6,113,906; 6,127,355; 6,132,713; 6,177,087, and 6,180,095. The structure of the conjugates of the antibody-polyalkylene glycols via the bridge linker is as following Pg01, Pg02, and Pg03.

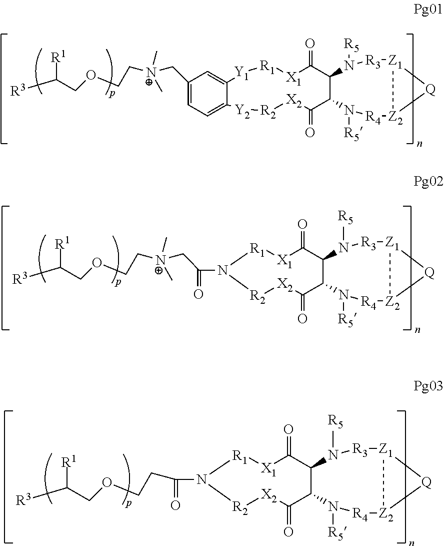

wherein “-----”, Q, X₁, X₂, Y₁, Y₂, R₁, R₂, R₃, R₄, R₅, R_{5′}, Z₁, Z₂, and n are defined the same above; Preferably Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; p is 1-5000; R¹ and R³ are defined the same as R₁ above, and preferably R¹ and R³ are H, OH, OCH₃, CH₃, or OC₂H₅ independently.

In yet another embodiment, the preferred cytotoxic agents that conjugated to a cell-binding molecule via a bridge linker of this patent are tubulysins, maytansinoids, taxanoids (taxanes), CC-1065 analogs, daunorubicin and doxorubicin compounds, amatoxins (including amanitins), indolecarboxamide, benzodiazepine dimers (e.g., dimers of pyrrolobenzodiazepine (PBD), tomaymycin, anthramycin, indolinobenzodiazepines, imidazobenzothiadiazepines, or oxazolidinobenzodiazepines), calicheamicins and the enediyne antibiotics, actinomycin, azaserines, bleomycins, epirubicin, eribulin, tamoxifen, idarubicin, dolastatins, auristatins (e.g. monomethyl auristatin E, MMAE, MMAF, auristatin PYE, auristatin TP, Auristatins 2-AQ, 6-AQ, EB (AEB), and EFP (AEFP) and their analogs), duocarmycins, geldanamycins or other HSP90 inhibitors, centanamycin, methotrexates, thiotepa, vindesines, vincristines, erbulins, hemiasterlins, nazumamides, microginins, radiosumins, streptonigtin, SN38 or other analogs or metabolites of camptothecin, alterobactins, microsclerodermins, theonellamides, esperamicins, PNU-159682; and their analogues or derivatives, pharmaceutically acceptable salts, acids, derivatives, hydrate or hydrated salt; or a crystalline structure; or an optical isomer, racemate, diastereomer or enantiomer of any of the above drugs thereof.

Tubulysins that are preferred for conjugation in the present invention are well known in the art and can be isolated from natural sources according to known methods or prepared synthetically according to known methods (e. g. Balasubramanian, R., et al. J. Med. Chem., 2009, 52, 238-40; Wipf, P., et al. Org. Lett., 2004, 6, 4057-60; Pando, O., et al. J. Am. Chem. Soc., 2011, 133, 7692-5; Reddy, J. A., et al. Mol. Pharmaceutics, 2009, 6, 1518-25; Raghavan, B., et al. J. Med. Chem., 2008, 51, 1530-33; Patterson, A. W., et al. J. Org. Chem., 2008, 73, 4362-9; Pando, O., et al. Org. Lett., 2009, 11 (24), 5567-9; Wipf, P., et al. Org. Lett., 2007, 9 (8), 1605-7; Friestad, G. K., Org. Lett., 2004, 6, 3249-52; Peltier, H. M., et al. J. Am. Chem. Soc., 2006, 128, 16018-9; Chandrasekhar, S., et al J. Org. Chem., 2009, 74, 9531-4; Liu, Y., et al. Mol. Pharmaceutics, 2012, 9, 168-75; Friestad, G. K., et al. Org. Lett., 2009, 11, 1095-8; Kubicek, K., et al., Angew Chem Int Ed Engl, 2010.49: 4809-12; Chai, Y., et al., Chem Biol, 2010, 17: 296-309; Ullrich, A., et al., Angew Chem Int Ed Engl, 2009, 48, 4422-5; Sani, M., et al. Angew Chem Int Ed Engl, 2007, 46, 3526-9; Domling, A., et al., Angew Chem Int Ed Engl, 2006, 45, 7235-9; patent applications: Zanda, M., et al, Can. Pat. Appl. CA 2710693 (2011); Chai, Y., et al. Eur. Pat. Appl. 2174947 (2010), WO2010034724; Leamon, C. et al, WO2010033733, WO 2009002993; Ellman, J., et al, PCT WO2009134279; WO 2009012958, US appl. 20110263650, 20110021568; Matschiner, G., et al, WO2009095447; Vlahov, I., et al, WO2009055562, WO 2008112873; Low, P., et al, WO2009026177; Richter, W., WO2008138561; Kjems, J., et al, WO 2008125116; Davis, M.; et al, WO2008076333; Diener, J.; et al, U.S. Pat. Appl. 20070041901, WO2006096754; Matschiner, G., et al, WO2006056464; Vaghefi, F., et al, WO2006033913; Doemling, A., Ger. Offen. DE102004030227, WO2004005327, WO2004005326, WO2004005269; Stanton, M., et al, U.S. Pat. Appl. Publ. 20040249130; Hoefle, G., et al, Ger. Offen. DE10254439, DE10241152, DE10008089; Leung, D., et al, WO2002077036; Reichenbach, H., et al, Ger. Offen. DE19638870; Wolfgang, R., US20120129779; Chen, H., US appl. 20110027274. The preferred structures of tubulysins for conjugation of cell binding molecules are described in the patent application of PCT/IB2012/053554.

Examples of the structures of the conjugates of the antibody-tubulysin analogs via a bis-linker are T01, T02, T03, T04, T05, T06 T07, T08, T09, T10, T11, T12, T13, T14, T15, T16 T017, T18, T19, T20, T21, T22 and T23 as following:

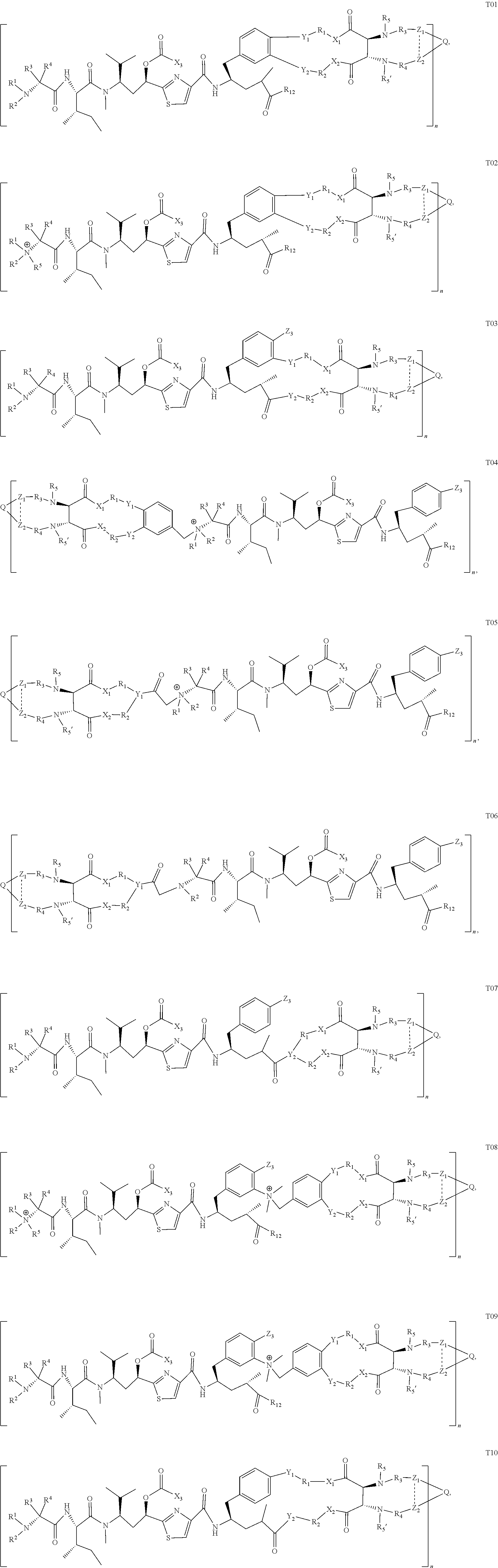

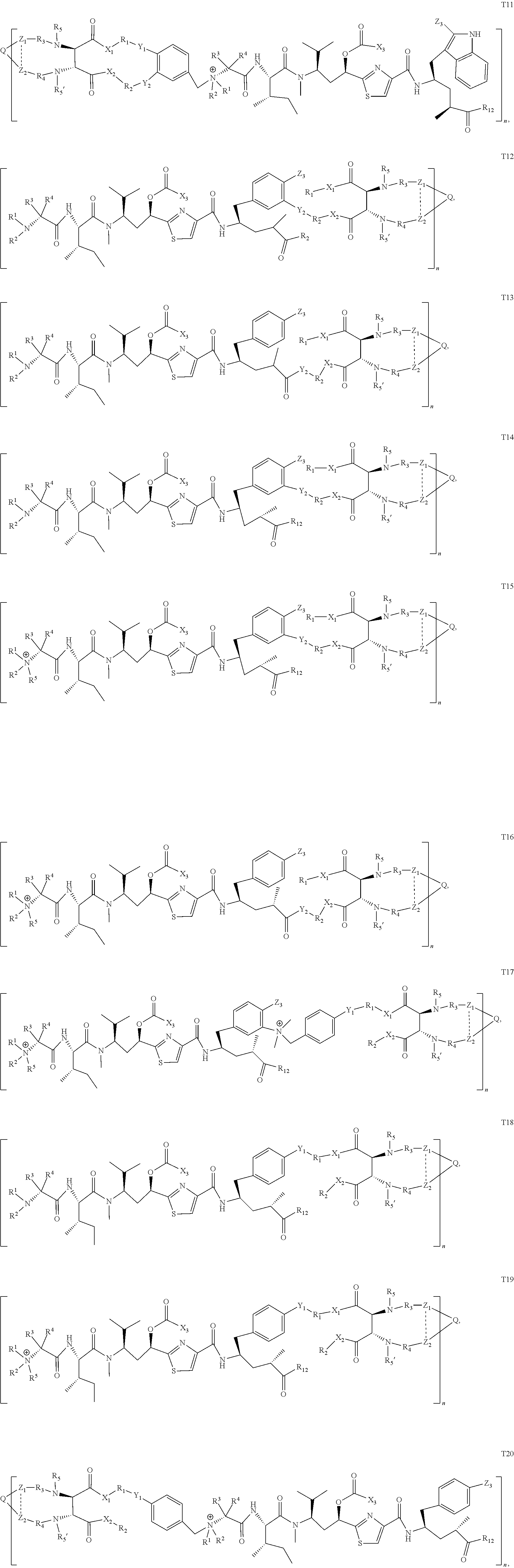

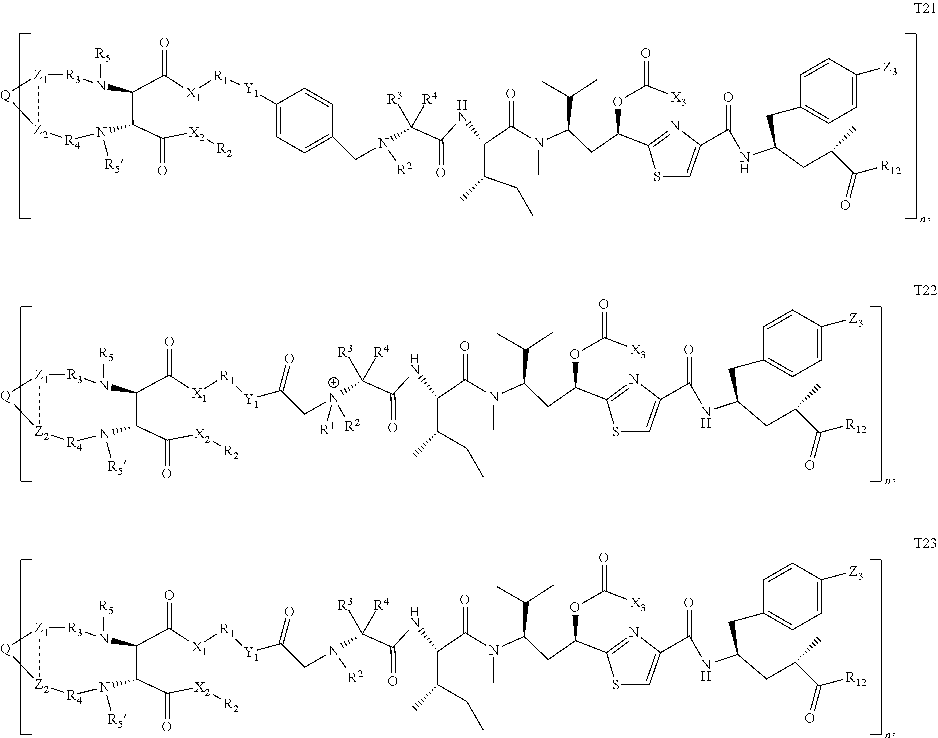

wherein “------”, Q, Y₁, Y₂, R₁, R₂, R₃, R₄, R₅, R_{5′}, Z₁, Z₂, and n are defined the same above; Preferably Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂CH₂NH₂, NH(CH₂CH₂S)ₚCH₂CH₂NH₂, NH(CH₂CH₂NH)ₚCH₂CH₂OH, NH(CH₂CH₂S)ₚCH₂CH₂OH, NH—R₁—NH₂, or NH(CH₂CH₂O)ₚCH₂CH₂NHPO₃H₂, wherein Aa is 1-8 aminoacids; n is 1-20; p is 1-5000; R¹, R^{1′}, R², R³, R⁴ and R⁵ are independently H, C₁-C₈ lineal or branched alkyl, amide, or amines; C₂-C₅ aryl, alkenyl, alkynyl, heteroaryl, heteroalkyl, alkylcycloalkyl, ester, ether, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000; The two Rs: R¹R², R²R³, R¹R³ or R³R⁴ can form 3˜8 member cyclic ring of alkyl, aryl, heteroaryl, heteroalkyl, or alkylcycloalkyl group; X₃ is H, CH₃, CH₂CH₃, C₃H₇, or X₁′R₁′, wherein X₁′ is NH, N(CH₃), NHNH, O, or S; R₁′ is H or C₁-C₈ lineal or branched alkyl, aryl, heteroaryl, heteroalkyl, alkylcycloalkyl, or acyloxylamines; R₃′ is H or C₁-C₆ lineal or branched alkyl; Z₃ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃.

Calicheamicins and their related enediyne antibiotics that are preferred for cell-binding molecule-drug conjugates of this patent are described in: Nicolaou, K. C. et al, Science 1992, 256, 1172-1178; Proc. Natl. Acad. Sci USA. 1993, 90, 5881-8), U.S. Pat. Nos. 4,970,198; 5,053,394; 5,108,912; 5,264,586; 5,384,412; 5,606,040; 5,712,374; 5,714,586; 5,739,116; 5,770,701; 5,770,710; 5,773,001; 5,877,296; 6,015,562; 6,124,310; 8,153,768. Examples of the structure of the conjugate of the antibody-Calicheamicin analog via the bridge linker are C01 and C02 as the following:

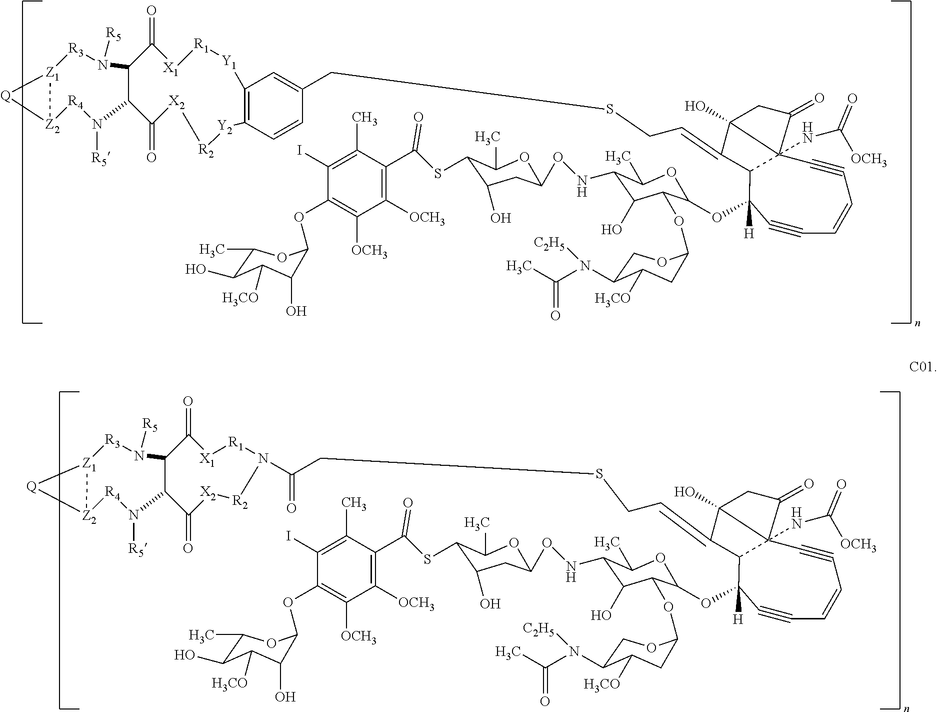

### C02

wherein “-----”, Q, Y₁, Y₂, R₁, R₂, R₃, R₄, R₅, R_{5′}, Z₁, Z₂, and n are defined the same above; Preferably X₁ X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; Q is preferably monoclonal antibody.

Maytansinoids that are preferred to be used in the present invention including maytansinol and its analogues are described in U.S. Pat. Nos. 4,256,746, 4,361,650, 4,307,016, 4,294,757, 4,294,757, 4,371,533, 4,424,219, 4,331,598, 4,450,254, 4,364,866, 4,313,946, 4,315,929 4,362,663, 4,322,348, 4,371,533, 4,424,219, 5,208,020, 5,416,064, 5,208,020; 5,416,064; 6,333,410; 6,441,163; 6,716,821, 7,276,497, 7,301,019, 7,303,749, 7,368,565, 7,411,063, 7,851,432, and 8,163,888. An example of the structure of the conjugate of the antibody-Maytansinoids via the linker of the patent is as the following My01, My02, My03, My04, My05, My06, My07, and My08:

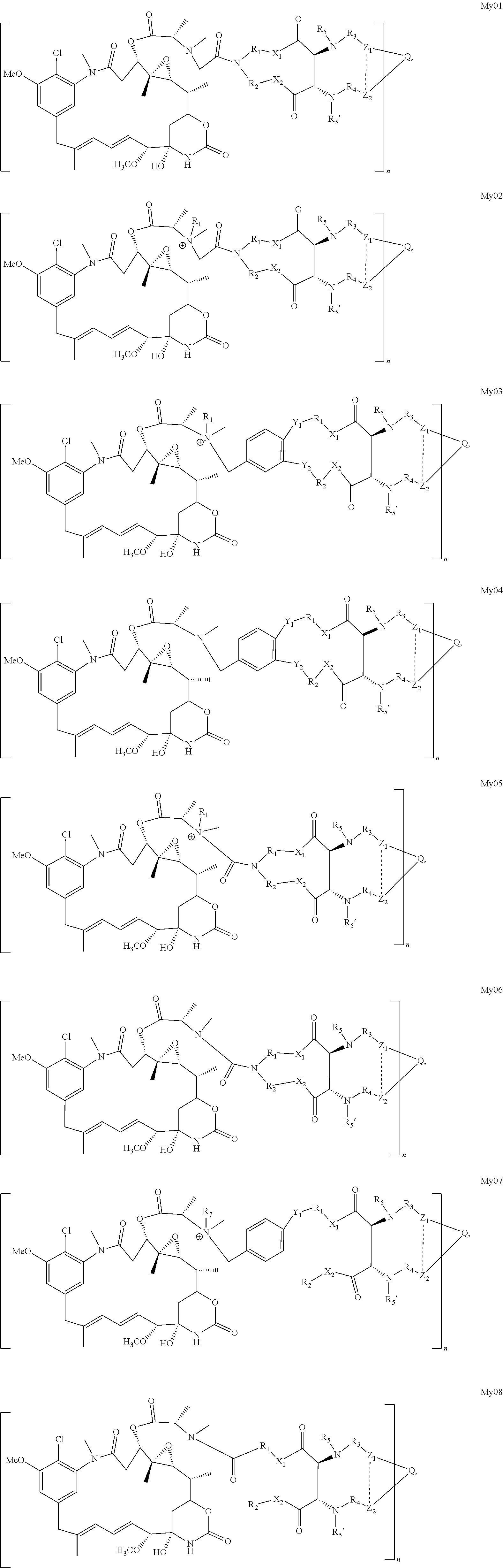

wherein “-----”, Q, Y₁, Y₂, R₁, R₂, R₃, R₄, R₅, R_{5′}, Z₁, Z₂, and n are defined the same above; Preferably X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; Q is preferably monoclonal antibody.

Taxanes, which includes Paclitaxel (Taxol), a cytotoxic natural product, and docetaxel (Taxotere), a semi-synthetic derivative, and their analogs which are preferred for conjugation are exampled in: K C. Nicolaou et al., J. Am. Chem. Soc. 117, 2409-20, (1995); Ojima et al, J. Med. Chem. 39:3889-3896 (1996); 40:267-78 (1997); 45, 5620-3 (2002); Ojima et al., Proc. Natl. Acad. Sci., 96:4256-61 (1999); Kim et al., Bull. Korean Chem. Soc., 20, 1389-90 (1999); Miller, et al. J. Med. Chem., 47, 4802-5(2004); U.S. Pat. No. 5,475,011 5,728,849, 5,811,452; 6,340,701; 6,372,738; 6,391,913, 6.436,931; 6,589,979; 6,596,757; 6,706,708; 7,008,942; 7,186,851; 7,217,819; 7,276,499; 7,598,290; and 7,667,054.

Examples of the structures of the conjugate of the antibody-taxanes via the linker of the patent are as the following Tx01, Tx02 and Tx03.

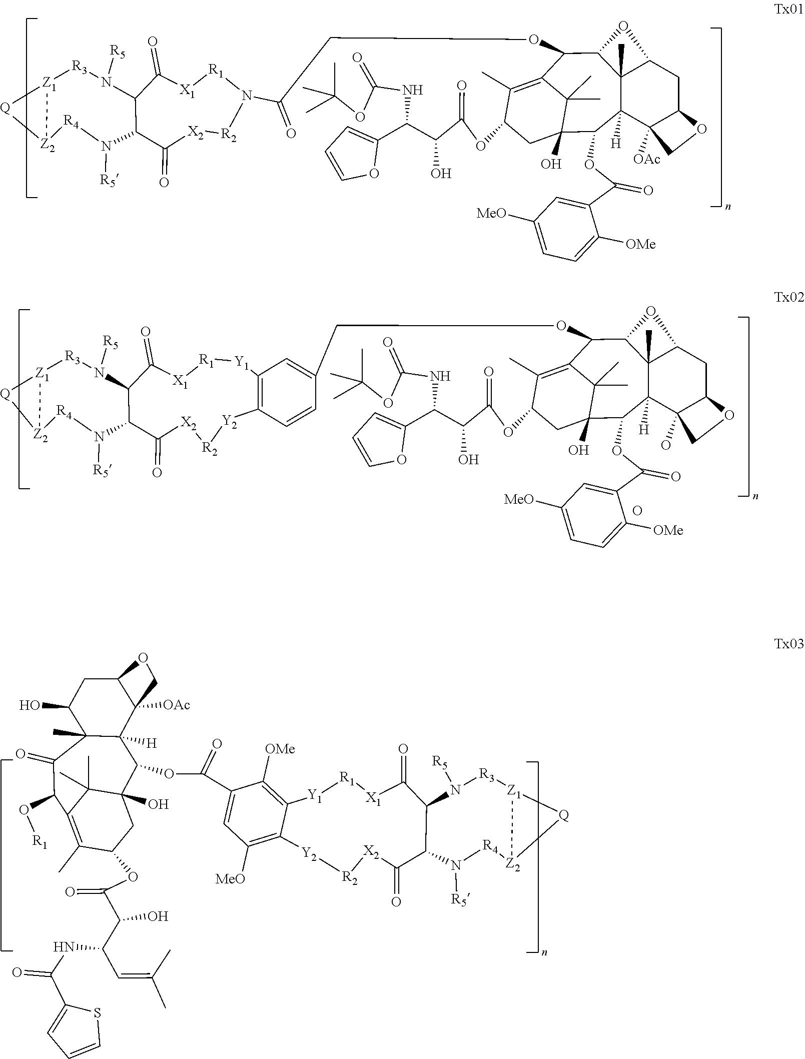

wherein “-----”, Q, Y₁, Y₂, R₁, R₂, R₃, R₄, R₅, R_{5′}, Z₁, Z₂, an n are defined the same above; Preferably X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; Q is preferably monoclonal antibody.

CC-1065 analogues and doucarmycin analogs are also preferred to be used for a conjugate containing bis-bridge linkage of the present patent. The examples of the CC-1065 analogues and doucarmycin analogs as well as their synthesis are described in: e.g. Warpehoski, et al, J. Med. Chem. 31:590-603 (1988); D. Boger et al., J. Org. Chem; 66; 6654-61, 2001; U.S. Pat. Nos. 4,169,888, 4,391,904, 4,671,958, 4,816,567, 4,912,227, 4,923,990, 4,952,394, 4,975,278, 4,978,757, 4,994,578, 5,037,993, 5,070,092, 5,084,468, 5,101,038, 5,117,006, 5,137,877, 5,138,059, 5,147,786, 5,187,186, 5,223,409, 5,225,539, 5,288,514, 5,324,483, 5,332,740, 5,332,837, 5,334,528, 5,403,484, 5,427,908, 5,475,092, 5,495,009, 5,530,101, 5,545,806, 5,547,667, 5,569,825, 5,571,698, 5,573,922, 5,580,717, 5,585,089, 5,585,499, 5,587,161, 5,595,499, 5,606,017, 5,622,929, 5,625,126, 5,629,430, 5,633,425, 5,641,780, 5,660,829, 5,661,016, 5,686,237, 5,693,762, 5,703,080, 5,712,374, 5,714,586, 5,739,116, 5,739,350, 5,770,429, 5,773,001, 5,773,435, 5,786,377 5,786,486, 5,789,650, 5,814,318, 5,846,545, 5,874,299, 5,877,296, 5,877,397, 5,885,793, 5,939,598, 5,962,216, 5,969,108, 5,985,908, 6,060,608, 6,066,742, 6,075,181, 6,103,236, 6,114,598, 6,130,237, 6,132,722, 6,143,901, 6,150,584, 6,162,963, 6,172,197, 6,180,370, 6,194,612, 6,214,345, 6,262,271, 6,281,354, 6,310,209, 6,329,497, 6,342,480, 6,486,326, 6,512,101, 6,521,404, 6,534,660, 6,544,731, 6,548,530, 6,555,313, 6,555,693, 6,566,336, 6,586,618, 6,593,081, 6,630,579, 6,756,397, 6,759,509, 6,762,179, 6,884,869, 6,897,034, 6,946,455, 7,049,316, 7,087,600, 7,091,186, 7,115,573, 7,129,261, 7,214,663, 7,223,837, 7,304,032, 7,329,507, 7,329,760, 7,388,026, 7,655,660, 7,655,661, 7,906,545, and 8,012,978. Examples of the structures of the conjugate of the antibody-CC-1065 analogs via the linker of the patent are as the following CC01, CC02, CC03, CC04, CC05, CC06 and CC07.

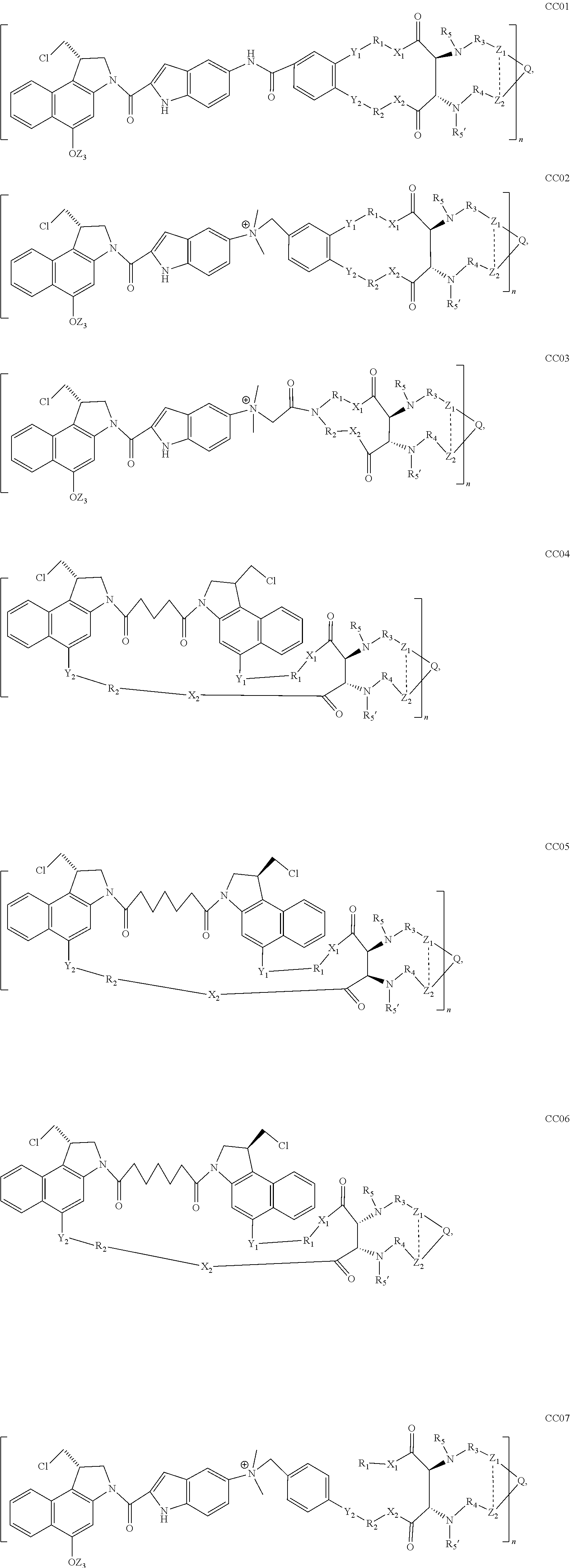

wherein “-----”, Q, Y₁, Y₂, R₁, R₂, R₃, R₄, R₅, R_{5′}, Z₁, Z₂, and n are defined the same above; Preferably X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; Q is preferably monoclonal antibody; Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside.

Daunorubicin/Doxorubicin Analogues are also preferred for conjugation having the bis-linkage of the present patent. The preferred structures and their synthesis are exampled in: Hurwitz, E., et al., Cancer Res. 35, 1175-81 (1975). Yang, H. M., and Reisfeld, R. A., Proc. Natl. Acad. Sci. 85, 1189-93 (1988); Pietersz, C. A., E., et al., E., et al., “Cancer Res. 48, 926-311 (1988); Trouet, et al., 79, 626-29 (1982); Z. Brich et al., J. Controlled Release, 19, 245-58 (1992); Chen et al., Syn. Comm., 33, 2377-90, 2003; King et al., Bioconj. Chem., 10, 279-88, 1999; King et al., J. Med. Chem., 45, 4336-43, 2002; Kratz et al., J Med Chem. 45, 5523-33, 2002; Kratz et al., Biol Pharm Bull. January 21, 56-61, 1998; Lau et al., Bioorg. Med. Chem. 3, 1305-12, 1995; Scott et al., Bioorg. Med. Chem. Lett. 6, 1491-6, 1996; Watanabe et al., Tokai J. Experimental Clin. Med. 15, 327-34, 1990; Zhou et al., J. Am. Chem. Soc. 126, 15656-7, 2004; WO 01/38318; U.S. Pat. Nos. 5,106,951; 5,122,368; 5,146,064; 5,177,016; 5,208,323; 5,824,805; 6,146,658; 6,214,345; 7,569,358; 7,803,903; 8,084,586; 8,053,205. Examples of the structures of the conjugate of the antibody-CC-1065 analogs via the linker of the patent are as the following Da01, Da02, Da03, Da04, Da05, Da06, Da07, Da08, Da09, Da10, and Da11.

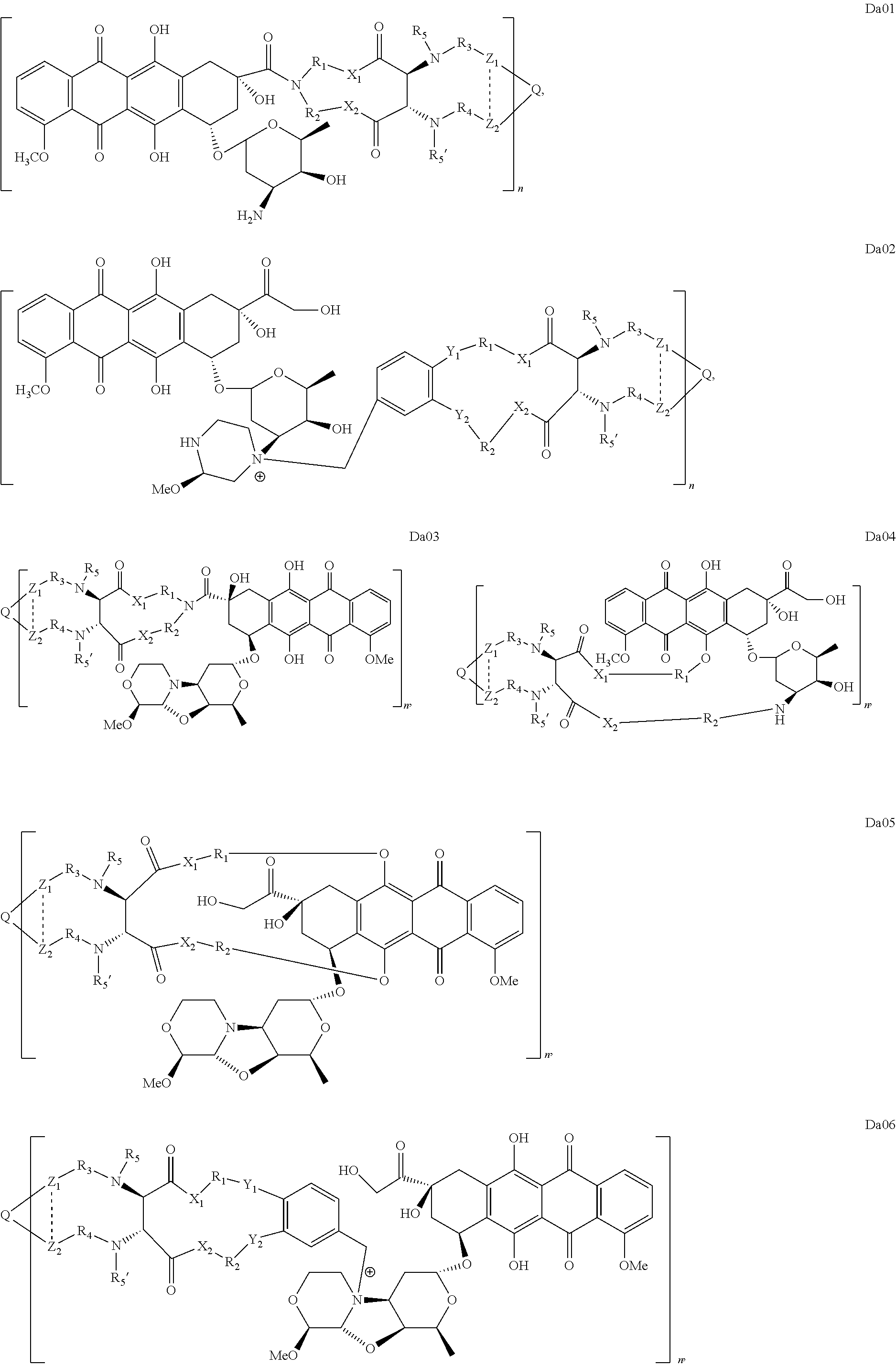

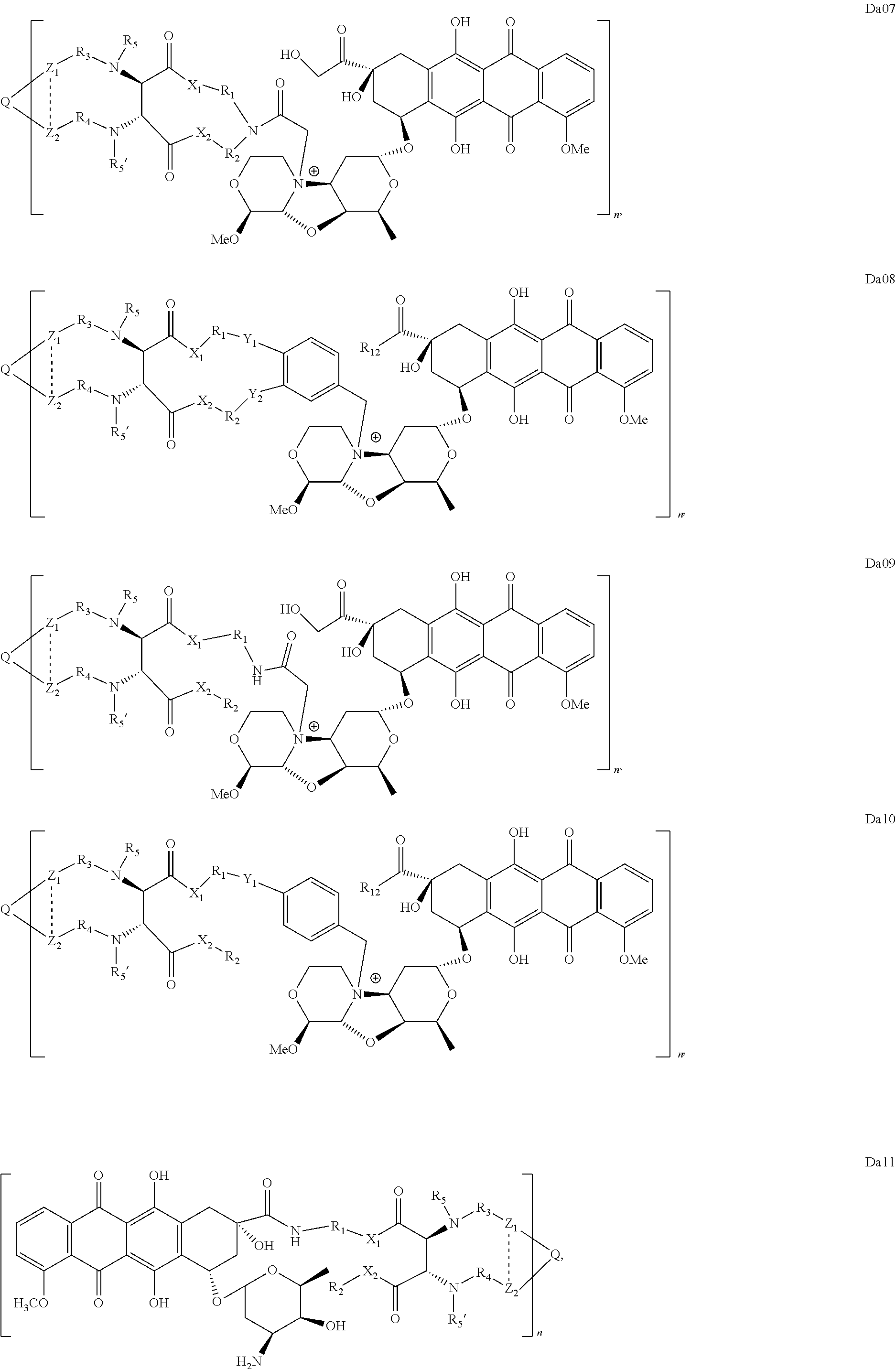

wherein “-----”, Q, Y₁, Y₂, R₁, R₂, R₃, R₄, R₅, R_{5′}, Z₁, Z₂, and n are defined the same above; Preferably X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R₁₂ is OH, NH₂, NHR₁, NHNH₂, NHNHCOOH, O—R₁—COOH, NH—R₁—COOH, NH(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R_{1′}, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NH—SO₃H, R₁—NHSO₃H, NH—R₁—NHSO₃H, O(CH₂CH₂O)ₚCH₂—CH₂NHPO₃H₂, NH(CH₂CH₂O)ₚCH₂—CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 aminoacids; p is 1-5000; Q is antibody, preferably monoclonal antibody.

Auristatins and dolastatins are preferred in conjugation containing the bis-linkers of this patent. The auristatins (e. g. auristatin E (AE) auristatin EB (AEB), auristatin EFP (AEFP), monomethyl auristatin E (MMAE), Monomethylauristatin (MMAF), Auristatin F phenylene diamine (AFP) and a phenylalanine variant of MMAE) which are synthetic analogs of dolastatins, are described in Int. J. Oncol. 15: 367-72 (1999); Molecular Cancer Therapeutics, vol. 3, No. 8, pp. 921-32 (2004); U.S. Application Nos. 11134826, 20060074008, 2006022925. U.S. Pat. Nos. 4,414,205, 4,753,894, 4,764,368, 4,816,444, 4,879,278, 4,943,628, 4,978,744, 5,122,368, 5,165,923, 5,169,774, 5,286,637, 5,410,024, 5,521,284, 5,530,097, 5,554,725, 5,585,089, 5,599,902, 5,629,197, 5,635,483, 5,654,399, 5,663,149, 5,665,860, 5,708,146, 5,714,586, 5,741,892, 5,767,236, 5,767,237, 5,780,588, 5,821,337, 5,840,699, 5,965,537, 6,004,934, 6,033,876, 6,034,065, 6,048,720, 6,054,297, 6,054,561, 6,124,431, 6,143,721, 6,162,930, 6,214,345, 6,239,104, 6,323,315, 6,342,219, 6,342,221, 6,407,213, 6,569,834, 6,620,911, 6,639,055, 6,884,869, 6,913,748, 7,090,843, 7,091,186, 7,097,840, 7,098,305, 7,098,308, 7,498,298, 7,375,078, 7,462,352, 7,553,816, 7,659,241, 7,662,387, 7,745,394, 7,754,681, 7,829,531, 7,837,980, 7,837,995, 7,902,338, 7,964,566, 7,964,567, 7,851,437, 7,994,135. Examples of the structures of the conjugate of the antibody-auristatins via the linker of the patent are as the following Au01, Au02, Au03, Au04, Au05, Au06, Au07, Au08, Au09, Au10, Au11, Au12, Au13, Au14, Au15, Au16, Au17, Au18, Au19, Au20, Au21, Au22, Au23, Au24, Au25, Au26, and Au27:

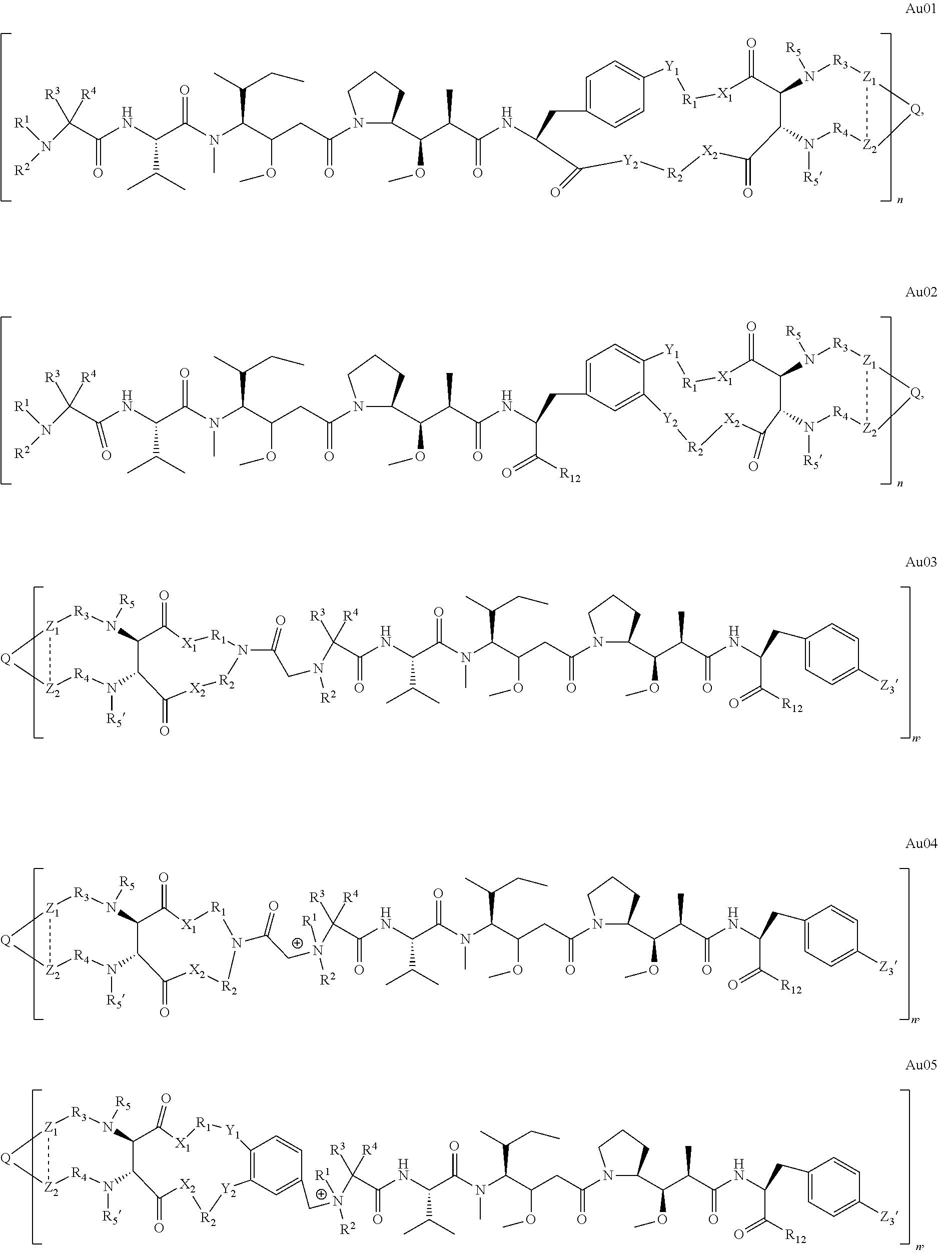

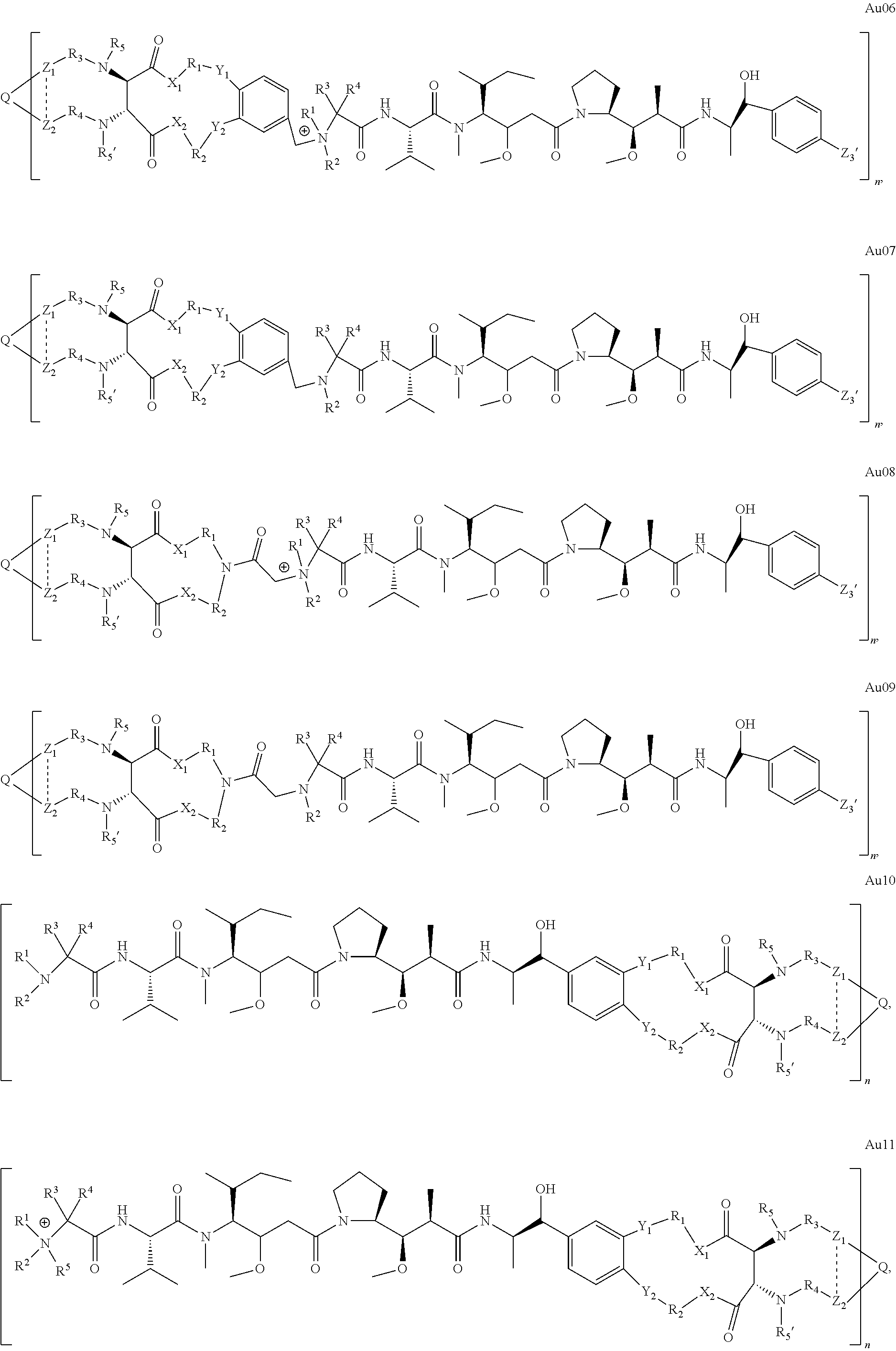

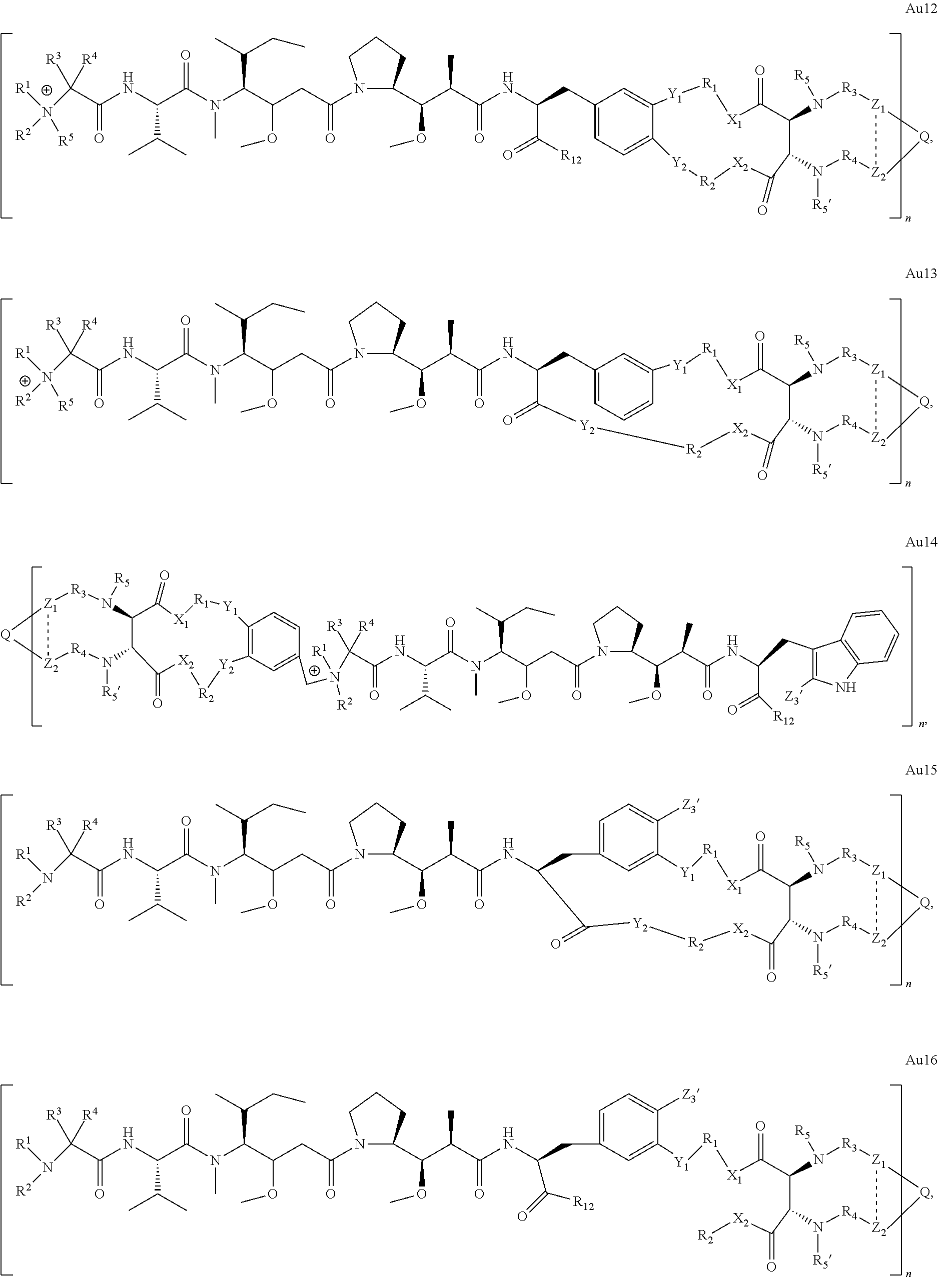

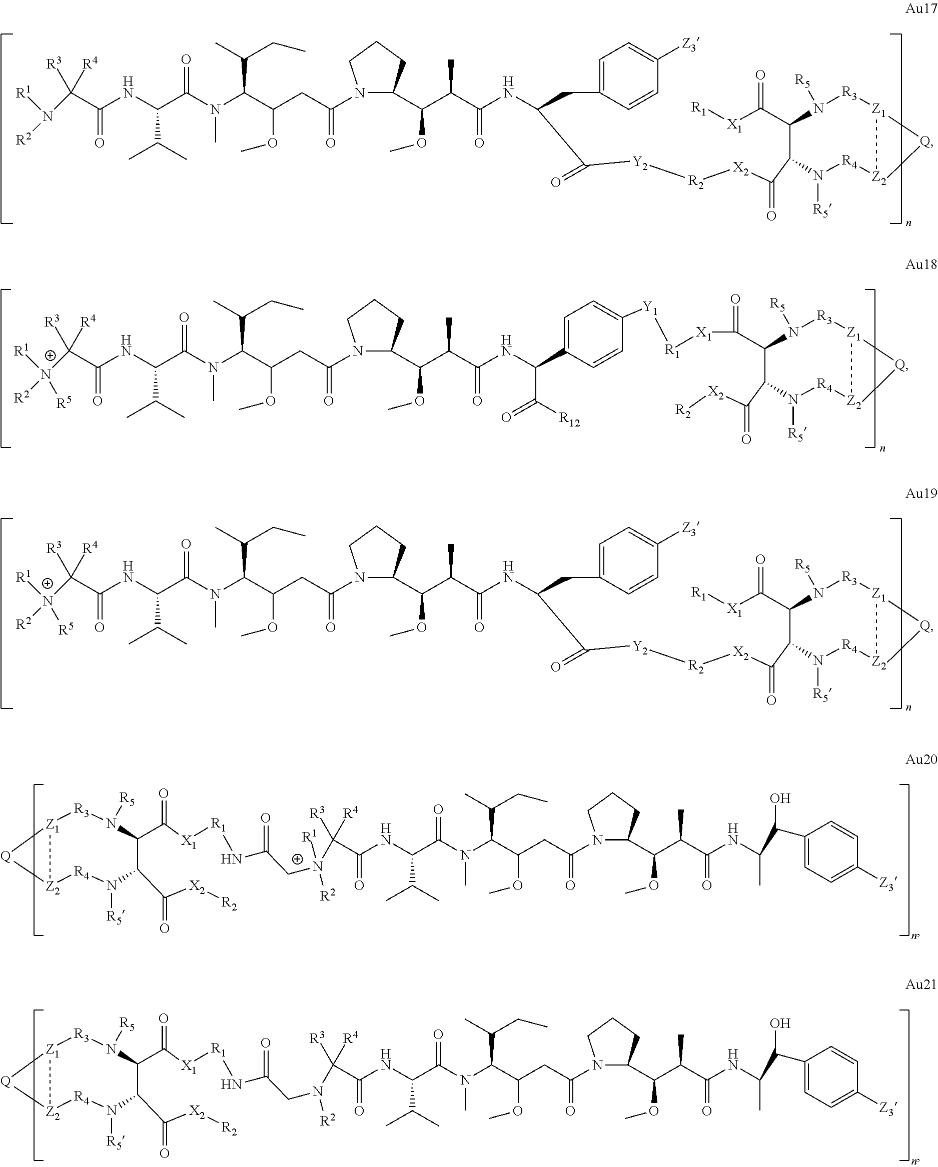

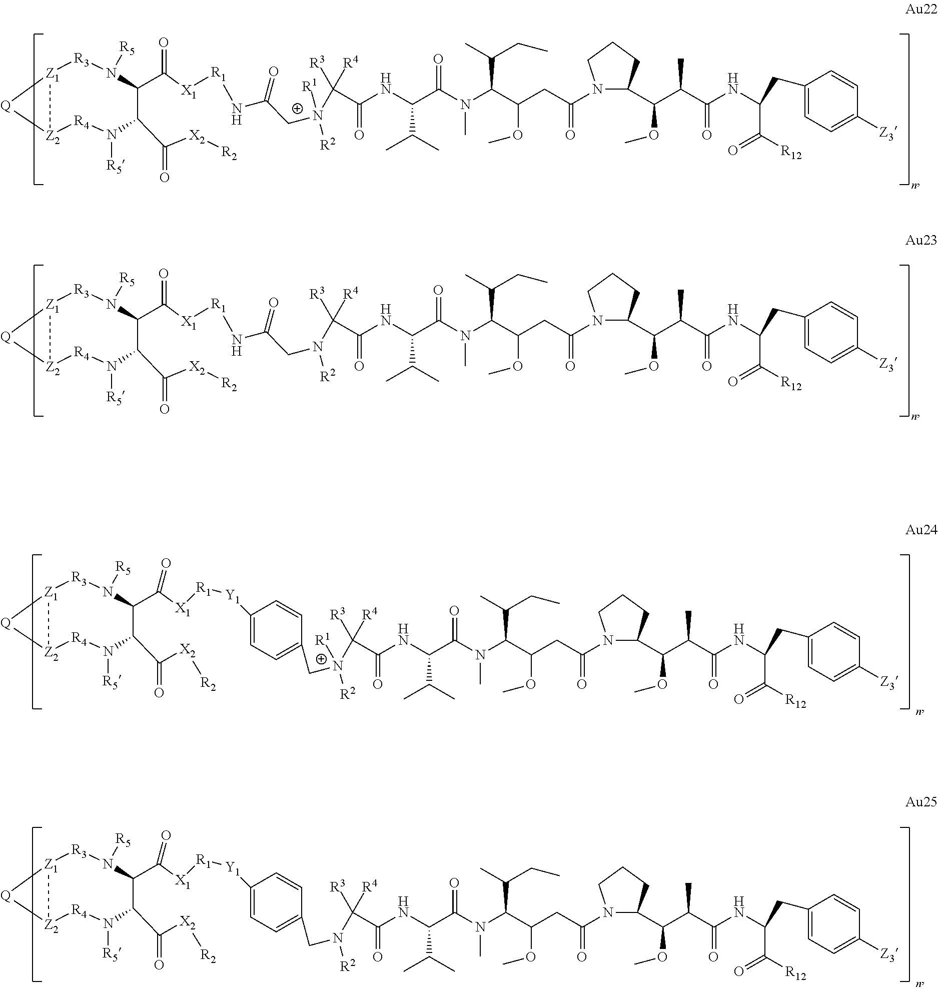

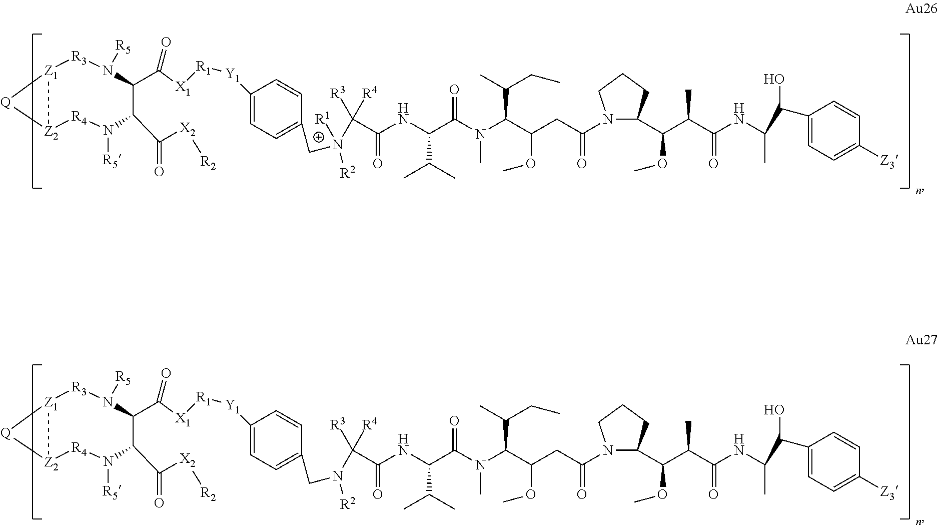

wherein “-----”, Q, Y₁, Y₂, R₁, R₂, R₃, R₄, R₅, R_{5′}, Z₁, Z₂, and n are defined the same above; Preferably X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH2, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH2, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH2, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 aminoacids; p is 1-5000; Q is preferably monoclonal antibody; R¹, R², R³, R⁴ and R⁵ are independently H; C₁-C₈ lineal or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000. The two Rs: R¹R², R²R³, R¹R³ or R³R⁴ can form 3˜8 member cyclic ring of alkyl, aryl, heteroaryl, heteroalkyl, or alkylcycloalkyl group; X₃ is H, CH₃ or X₁′R₁′, wherein X₁′ is NH, N(CH₃), NHNH, O, or S, and R₁′ is H or C₁-C₈ lineal or branched alkyl, aryl, heteroaryl, heteroalkyl, alkylcycloalkyl, acyloxylamines; R₃′ is H or C₁-C₆ lineal or branched alkyl; Z₃′ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃.

The benzodiazepine dimers (e. g. dimmers of pyrrolobenzodiazepine (PBD) or (tomaymycin), indolinobenzodiazepines, imidazobenzothiadiazepines, or oxazolidinobenzodiazepines) which are preferred cytotoxic agents according to the present invention are exampled in the art: U.S. Pat. Nos. 8,163,736; 8,153,627; 8,034,808; 7,834,005; 7,741,319; 7,704,924; 7,691,848; 7,678,787; 7,612,062; 7,608,615; 7,557,099; 7,528,128; 7,528,126; 7,511,032; 7,429,658; 7,407,951; 7,326,700; 7,312,210; 7,265,105; 7,202,239; 7,189,710; 7,173,026; 7,109,193; 7,067,511; 7,064,120; 7,056,913; 7,049,311; 7,022,699; 7,015,215; 6,979,684; 6,951,853; 6,884,799; 6,800,622; 6,747,144; 6,660,856; 6,608,192; 6,562,806; 6,977,254; 6,951,853; 6,909,006; 6,344,451; 5,880,122; 4,935,362; 4,764,616; 4,761,412; 4,723,007; 4,723,003; 4,683,230; 4,663,453; 4,508,647; 4,464,467; 4,427,587; 4,000,304; US patent appl. 20100203007, 20100316656, 20030195196. Examples of the structures of the conjugate of the antibody-benzodiazepine dimers via the bridge linker are as the following PB01, PB02, PB03, PB04, PB05, PB06, PB07, PB08, PB09, PB10, PB11, PB12, PB13, PB14, PB15, PB16, PB17, PB18, PB19, PB20, PB21, PB22, PB23, PB24, PB25, PB26, PB27, PB28, PB29, PB30, PB31 and PB32.

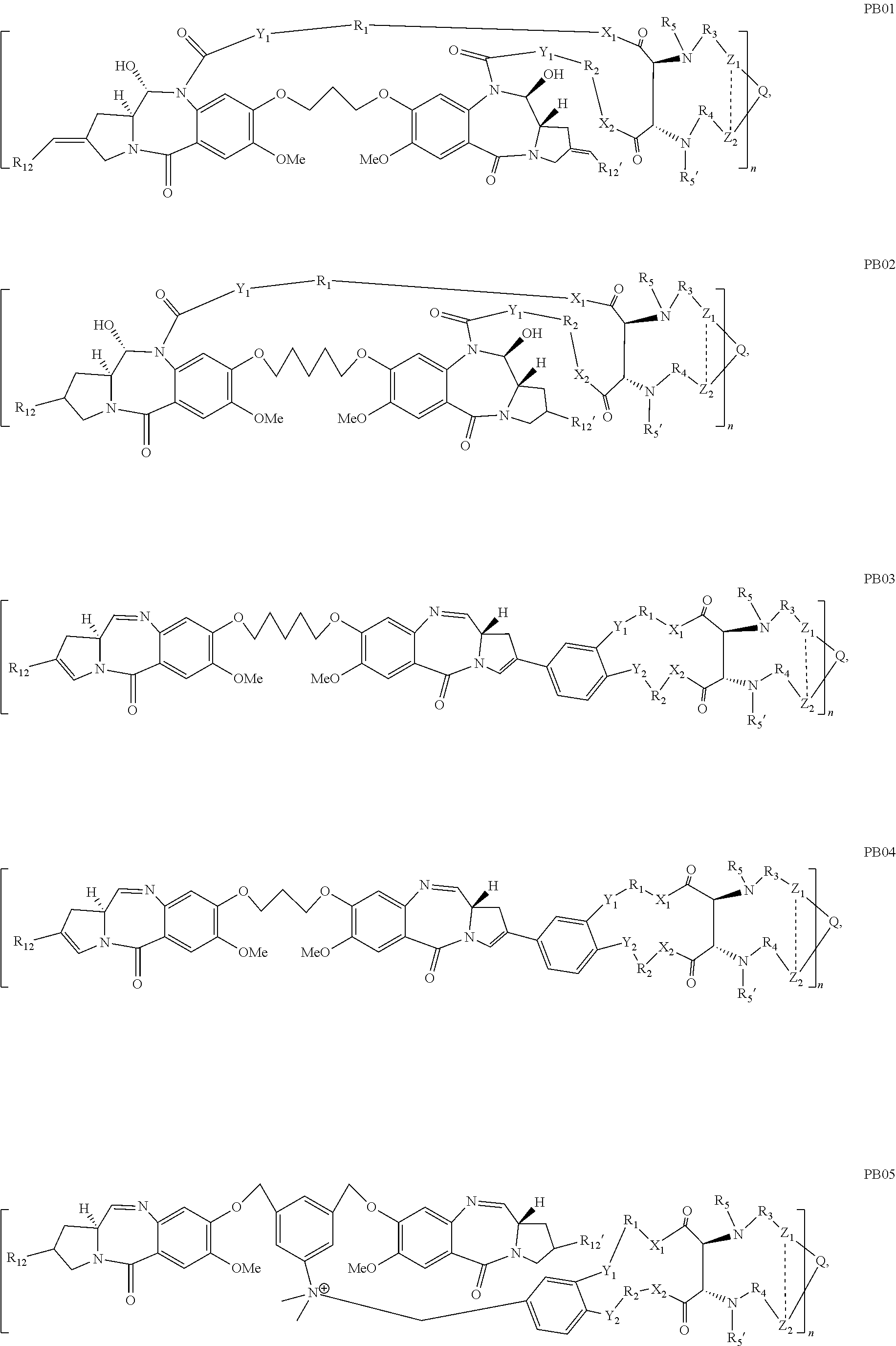

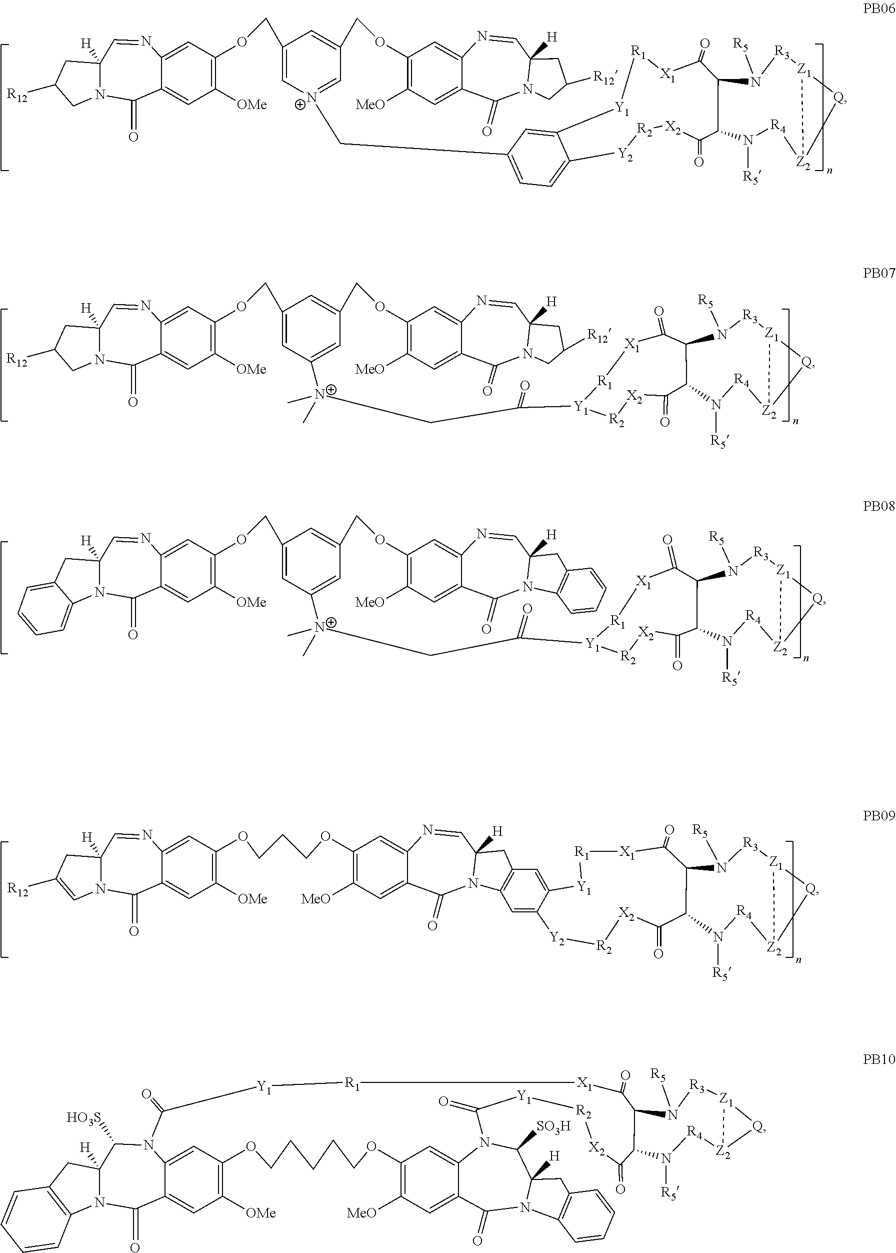

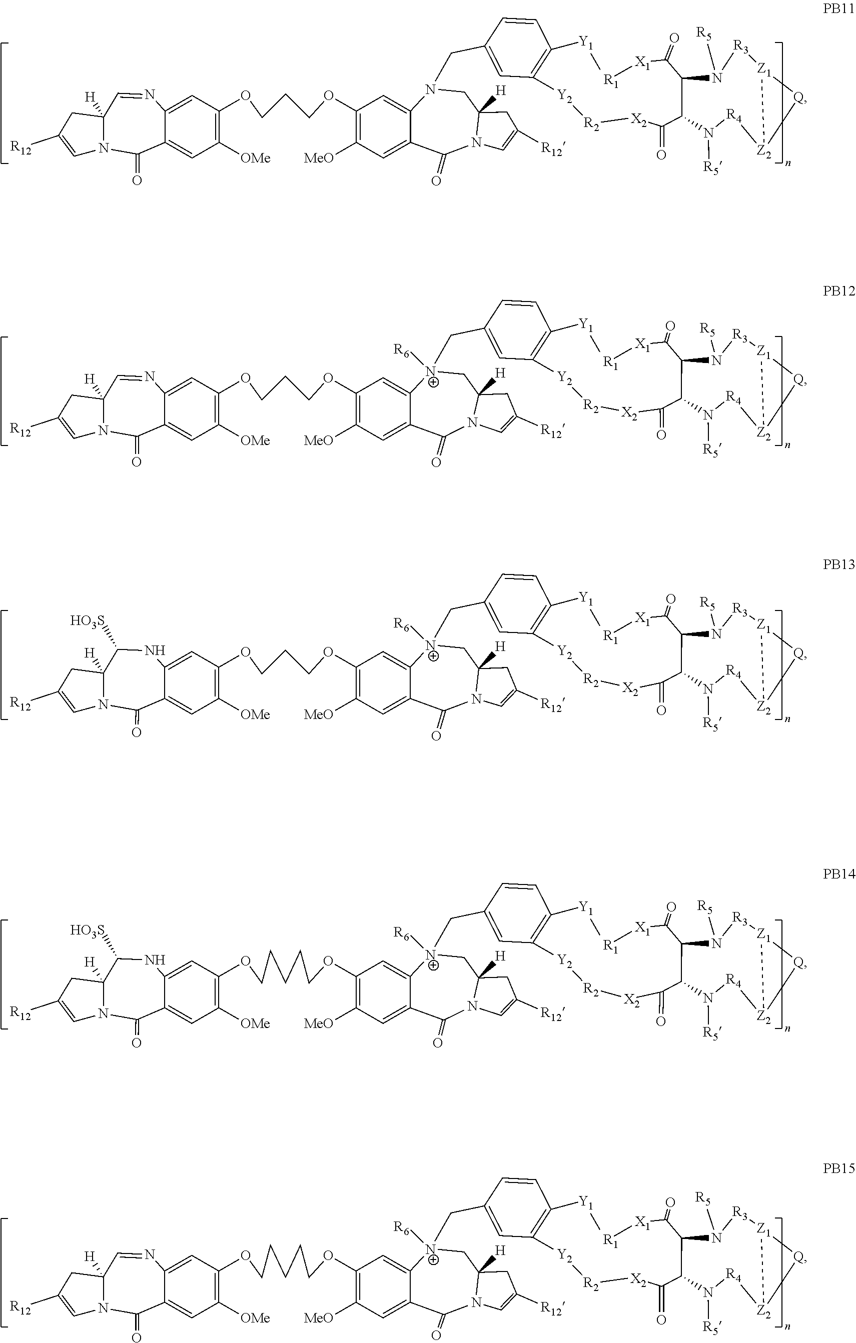

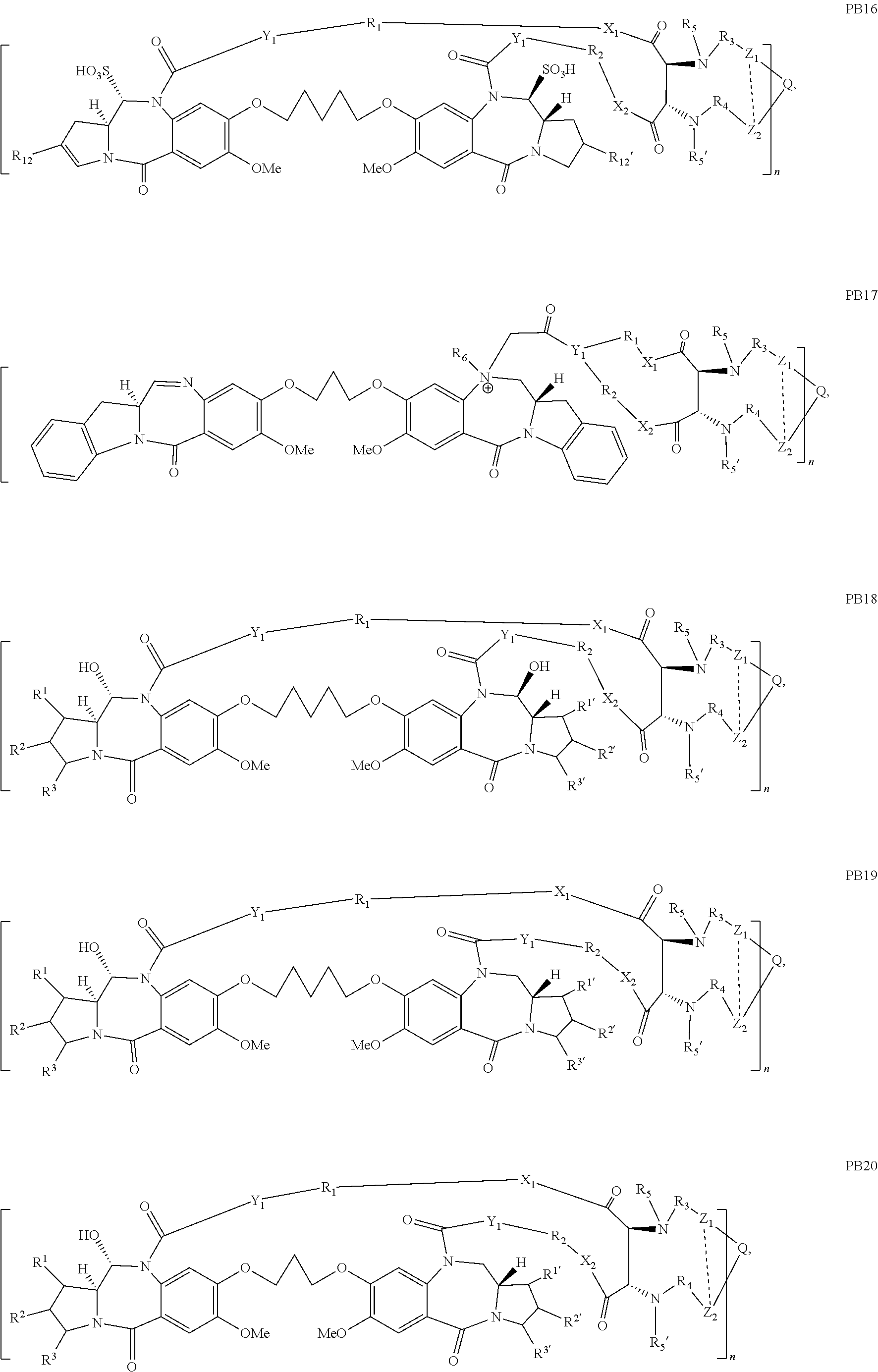

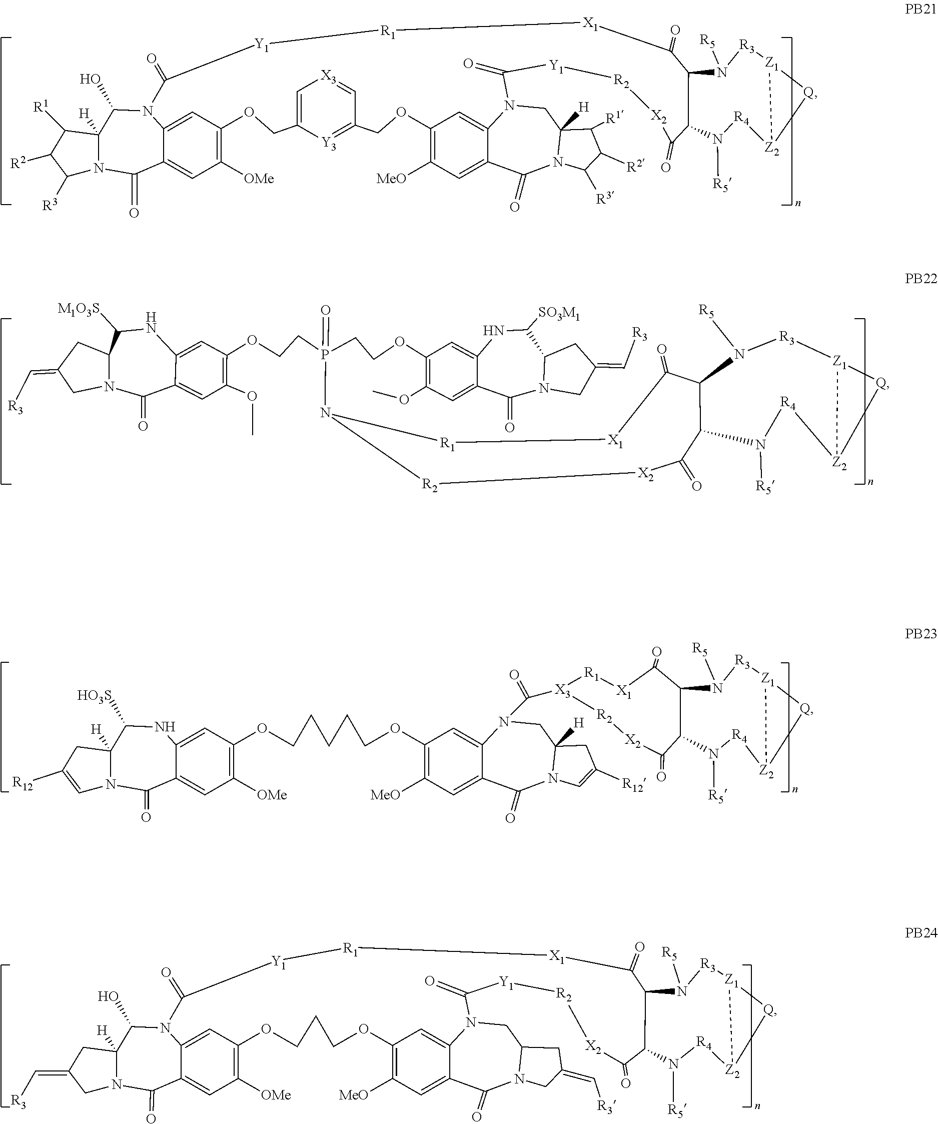

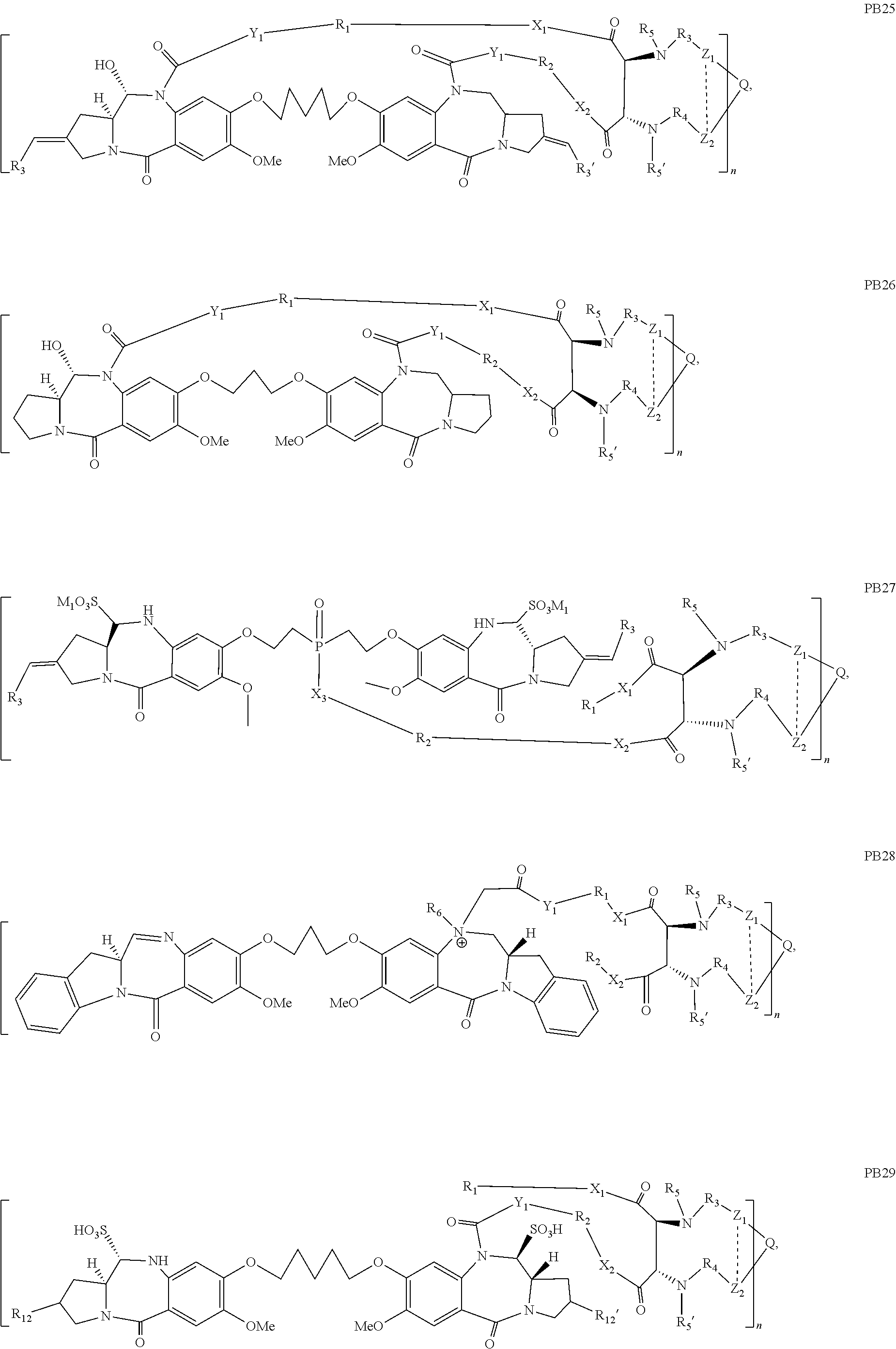

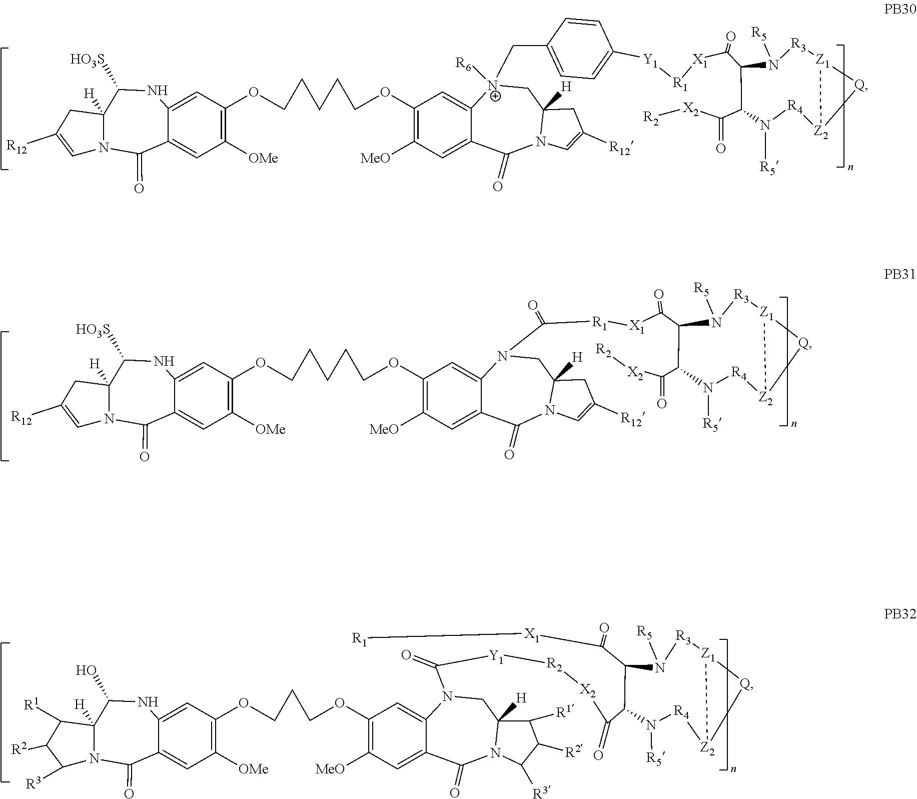

wherein “------”, Q, X₁, X₂, Y₁, Y₂, R₁, R₂, R₃, R₄, R₅, R₅′, Z₁, Z₂, and n are defined the same above; Preferably X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R¹, R², R³, R^{1′}, R^{2′}, and R^{3′} are independently H; F; Cl; ═O; ═S; OH; SH; C₁-C₈ lineal or branched alkyl, aryl, alkenyl, heteroaryl, heteroalkyl, alkylcycloalkyl, ester (COOR₅ or —OC(O)R₅), ether (OR₅), amide (CONR₅), carbamate (OCONR₅), amines (NHR₅, NR₅R₅′), heterocycloalkyl, or acyloxylamines (—C(O)NHOH, —ONHC(O)R₅); or peptides containing 1-20 natural or unnatural aminoacids, or polyethyleneoxy 15 unit of formula (OCH₂CH₂)ₚ or (OCH₂CH(CH₃))ₚ, wherein p is an integer from 1 to about 5000. The two Rs: R¹R², R²R³, R¹R³, R^{1′}R^{2′}, R^{2′}R^{3′}, or R^{1′}R^{3′} can independently form 3˜8 member cyclic ring of alkyl, aryl, heteroaryl, heteroalkyl, or alkylcycloalkyl group; X₃ and Y₃ are independently N, NH, CH₂ or CR₅, wherein R₅, R₆, R₁₂ and R₁₂′ are independently H, OH, NH₂, NH(CH₃), NHNH₂, COOH, SH, OZ₃, SZ₃, F, Cl, or C₁-C₈ lineal or branched alkyl, aryl, heteroaryl, heteroalkyl, alkylcycloalkyl, acyloxylamines; Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃.

Amatoxins which are a subgroup of at least ten toxic compounds originally found in several genera of poisonous mushrooms, most notably Amanita phalloides and several other mushroom species, are also preferred for conjugation of the present patent. These ten amatoxins, named α-Amanitin, β-Amanitin, γ-Amanitin, ε-Amanitin, Amanullin, Amanullinic acid, Amaninamide, Amanin, Proamanullin, are rigid bicyclic peptides that are synthesized as 35-amino-acid proproteins, from which the final eight amino acids are cleaved by a prolyl oligopeptidase (Litten, W. 1975 Scientific American 232 (3): 90-101; H. E. Hallen, et al 2007 Proc. Nat. Aca. Sci. USA 104, 19097-101; K. Baumann, et al, 1993 Biochemistry 32 (15): 4043-50; Karlson-Stiber C, Persson H. 2003, Toxicon 42 (4): 339-49; Horgen, P. A. et al. 1978 Arch. Microbio. 118 (3): 317-9). Amatoxins kill cells by inhibiting RNA polymerase II (Pol II), shutting down gene transcription and protein biosynthesis (Brodner, O. G. and Wieland, T. 1976 Biochemistry, 15(16): 3480-4; Fiume, L., Curr Probl Clin Biochem, 1977, 7: 23-8; Karlson-Stiber C, Persson H. 2003, Toxicon 42(4): 339-49; Chafin, D. R., Guo, H. & Price, D. H. 1995 J. Biol. Chem. 270 (32): 19114-19; Wieland (1983) Int. J. Pept. Protein Res. 22(3): 257-76). Amatoxins can be produced from collected Amanita phalloides mushrooms (Yocum, R. R. 1978 Biochemistry 17(18): 3786-9; Zhang, P. et al, 2005, FEMS Microbiol. Lett. 252(2), 223-8), or from fermentation using a basidiomycete (Muraoka, S. and Shinozawa T., 2000 J. Biosci. Bioeng. 89(1): 73-6) or from fermentation using A. fissa (Guo, X. W., et al, 2006 Wei Sheng Wu Xue Bao 46(3): 373-8), or from culturing Galerina fasciculata or Galerina helvoliceps, a strain belonging to the genus (WO/1990/009799, JP11137291). However the yields from these isolation and fermentation were quite low (less than 5 mg/L culture). Several preparations of amatoxins and their analogs have been reported in the past three decades (W. E. Savige, A. Fontana, Chem. Commun. 1976, 600-1; Zanotti, G., et al, Int J Pept Protein Res, 1981. 18(2): 162-8; Wieland, T., et al, Eur. J. Biochem. 1981, 117, 161-4; P. A. Bartlett, et al, Tetrahedron Lett. 1982, 23, 619-22; Zanotti, G., et al., Biochim Biophys Acta, 1986. 870(3): 454-62; Zanotti, G., et al., Int. J. Peptide Protein Res. 1987, 30, 323-9; Zanotti, G., et al., Int. J. Peptide Protein Res. 1987, 30, 450-9; Zanotti, G., et al., Int J Pept Protein Res, 1988. 32(1): 9-20; G. Zanotti, T. et al, Int. J. Peptide Protein Res. 1989, 34, 222-8; Zanotti, G., et al., Int J Pept Protein Res, 1990. 35(3): 263-70; Mullersman, J. E. and J. F. Preston, 3rd, Int J Pept Protein Res, 1991. 37(6): 544-51; Mullersman, J. E., et al, Int J Pept Protein Res, 1991. 38(5): 409-16; Zanotti, G., et al, Int J Pept Protein Res, 1992. 40(6): 551-8; Schmitt, W. et al, J. Am. Chem. Soc. 1996, 118, 4380-7; Anderson, M. O., et al, J. Org. Chem., 2005, 70(12): 4578-84; J. P. May, et al, J. Org. Chem. 2005, 70, 8424-30; F. Brueckner, P. Cramer, Nat. Struct. Mol. Biol. 2008, 15, 811-8; J. P. May, D. M. Perrin, Chem. Eur. J. 2008, 14, 3404-9; J. P. May, et al, Chem. Eur. J. 2008, 14, 3410-17; Q. Wang, et al, Eur. J. Org. Chem. 2002, 834-9; May, J. P. and D. M. Perrin, Biopolymers, 2007. 88(5): 714-24; May, J. P., et al., Chemistry, 2008. 14(11): 3410-7; S. De Lamo Marin, et al, Eur. J. Org. Chem. 2010, 3985-9; Pousse, G., et al., Org Lett, 2010. 12(16): 3582-5; Luo, H., et al., Chem Biol, 2014. 21(12): 1610-7; Zhao, L., et al., Chembiochem, 2015. 16(10): 1420-5) and most of these preparations were by partial synthesis. Because of their extreme potency and unique mechanism of cytotoxicity, amatoxins have been used as payloads for conjugations (Fiume, L., Lancet, 1969. 2 (7625): 853-4; Barbanti-Brodano, G. and L. Fiume, Nat New Biol, 1973. 243(130): 281-3; Bonetti, E., M. et al, Arch Toxicol, 1976. 35(1): p. 69-73; Davis, M. T., Preston, J. F. Science 1981, 213, 1385-1388; Preston, J. F., et al, Arch Biochem Biophys, 1981. 209(1): 63-71; H. Faulstich, et al, Biochemistry 1981, 20, 6498-504; Barak, L. S., et al., Proc Natl Acad Sci USA, 1981. 78(5): 3034-8; Faulstich, H. and L. Fiume, Methods Enzymol, 1985. 112: 225-37; Zhelev, Z., A. et al, Toxicon, 1987. 25(9): 981-7; Khalacheva, K., et al, Eksp Med Morfol, 1990. 29(3): 26-30; U. Bermbach, H. Faulstich, Biochemistry 1990, 29, 6839-45; Mullersman, J. E. and J. F. Preston, Int. J. Peptide Protein Res. 1991, 37, 544-51; Mullersman, J. E. and J. F. Preston, Biochem Cell Biol, 1991. 69(7): 418-27; J. Anderl, H. Echner, H. Faulstich, Beilstein J. Org. Chem. 2012, 8, 2072-84; Moldenhauer, G., et al, J. Natl. Cancer Inst. 2012, 104, 622-34; A. Moshnikova, et al; Biochemistry 2013, 52, 1171-8; Zhao, L., et al., Chembiochem, 2015. 16(10): 1420-5; Zhou, B., et al., Biosens Bioelectron, 2015. 68: 189-96; WO2014/043403, US20150218220, EP 1661584). We have been working on the conjugation of amatoxins for a while. Examples of the structures of the conjugate of the antibody-amatoxins via the bridge linker are preferred as the following structures of Am01, Am02, Am03, Am04, Am05, Am06, Am07, Am08 and Am09:

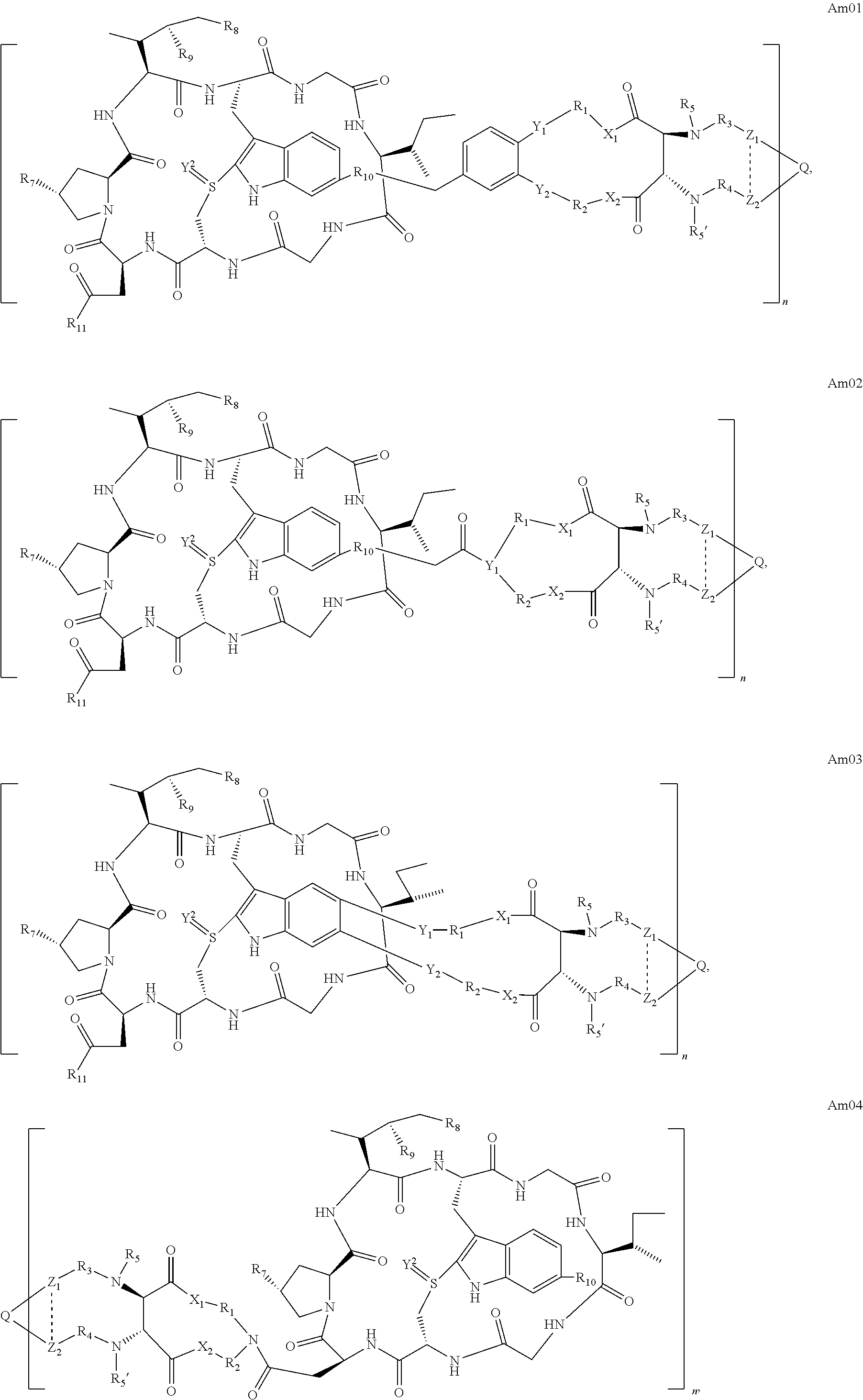

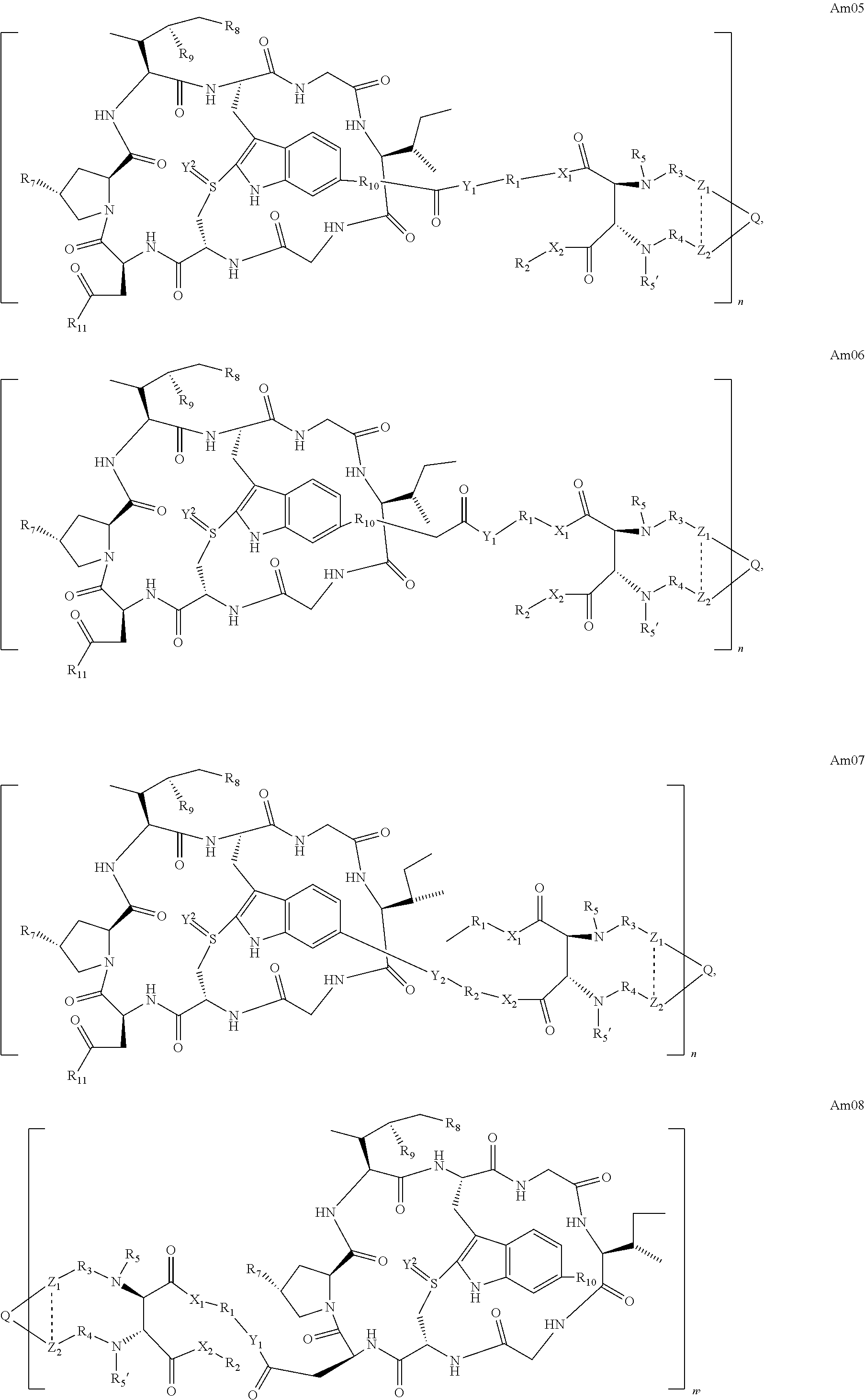

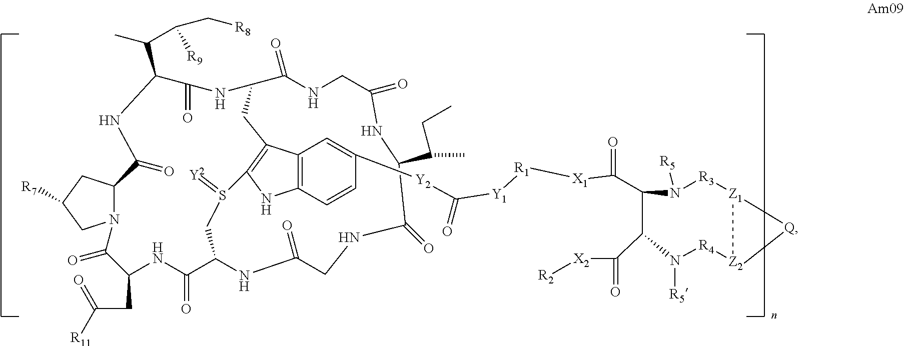

wherein “---”, X₁, X₂, Q, Y₁, Y₂, R₁, R₂, R₃, R₄, R₅, R_{5′}, Z₁, Z₂, and n are defined the same above; Preferably X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NH—NHC(O), C(O)NR₁ or absent; R₇, R₈, and R₉ are independently H, OH, OR₁, NH₂, NHR₁, C₁-C₆ alkyl, or absent; Y² is O, O₂, NR₁, NH, or absent; R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent; R₁ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, O(CH₂CH₂O)ₚCH₂CH₂COOH, NH(CH₂CH₂O)ₚCH₂—CH₂COOH, NH—Ar—COOH, NH—Ar—NH₂, O(CH₂CH₂O)ₚCH₂CH₂NHSO₃H, NH(CH₂CH₂—O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂C—H₂OPO₃H₂, OR₁—NHPO₃H₂, NH—R₁—NHPO₃H₂, or NH(CH₂CH₂O)ₚCH₂CH₂NHPO₃H₂, wherein Aa is 1-20 aminoacids; n and m₁ are independently 1-30; p is 1-5000; Z₃ is H, OH, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃.

Camptothecin (CPT) and its derivative SN-38, Topotecan, Irinotecan (CPT-11), Silatecan (DB-67, AR-67), Cositecan (BNP-1350), Etirinotecan, Exatecan, Lurtotecan, Gimatecan (ST1481), Belotecan (CKD-602), and Rubitecan are topoisomerase inhibitors that prevent DNA re-ligation and therefore causes DNA damage which results in apoptosis. So far two CPT analogues, topotecan and irinotecan have been approved and are used in cancer chemotherapy (Palakurthi, S., Expert Opin Drug Deliv. 2015; 12(12):1911-21) and some of them, such as SN-38 and Exatecan have been successfully used as payloads for ADC conjugates in the clinical trials (Ocean, A. J. et al, Cancer. 2017, 123(19): 3843-3854; Starodub, A. N., et al, Clin Cancer Res. 2015, 21(17): 3870-8; Cardillo, T. M., et al, Bioconjug Chem. 2015, 26(5): 919-31; Ogitani, Y. et al, Bioorg Med Chem Lett. 2016, 26(20): 5069-5072; Takegawa, N. et al, Int J Cancer. 2017 Oct. 15; 141(8):1682-1689. U.S. Pat. Nos. 7,591,994; 7,999,083, 8,080,250, 8,268,317; US patent applications 20130090458, 20140099258, 20150297748, 20160279259). Examples of the structures of the conjugate of the antibody-Camptothecin analogs via the bridge linker are preferred as the following structures of CP01, CP02, CP03, CP04, CP05, and CP06:

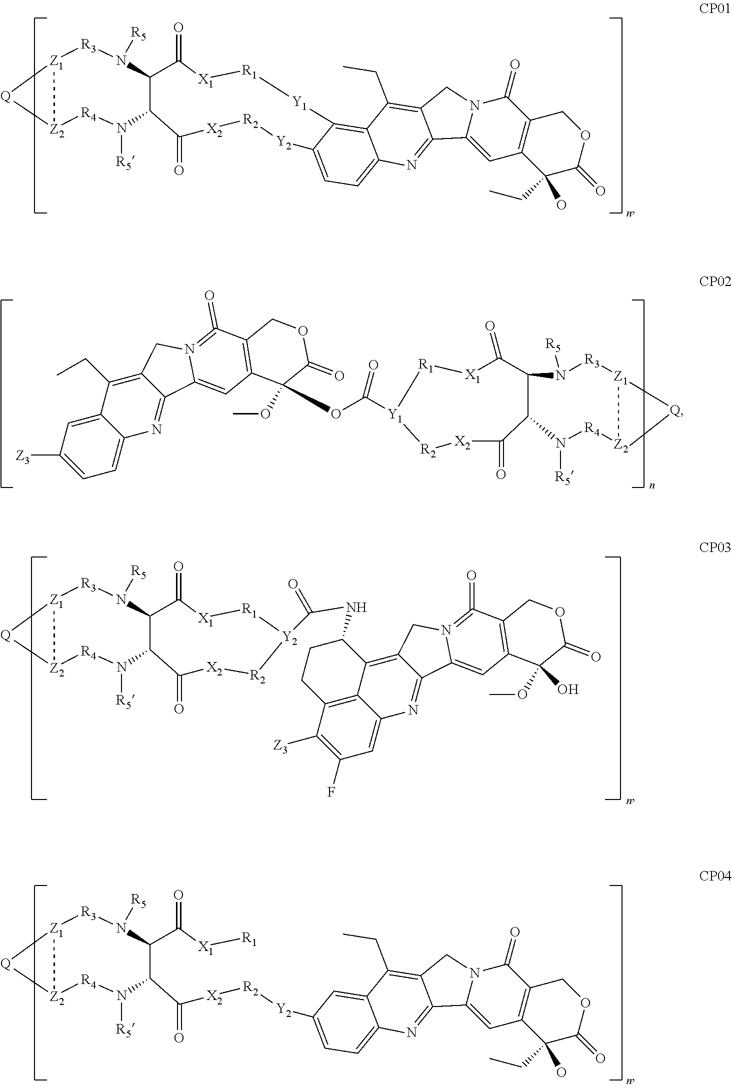

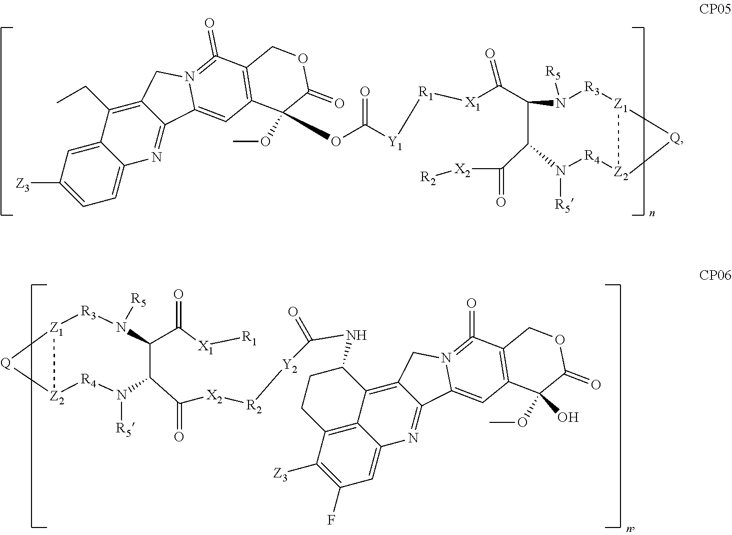

wherein “-----”, Q, X₁, X₂, Y₁, Y₂, R₁, R₂, R₃, R₄, R₅, R₅′, Z₁, Z₂, and n are defined the same above; Preferably X₁, X₂, Y₁ and Y₂ are independently O, N, NH, NHNH, NR₅, S, C(O)O, CH₂, C(O)NHNHC(O), C(O)NR₁ or absent; Z₃ is H, OH, COOR₁, NH₂, NHR₁, OR₁, CH₃, CONHR₁, NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃.

Eribulin which is binding predominantly to a small number of high affinity sites at the plus ends of existing microtubules has both cytotoxic and non-cytotoxic mechanisms of action. Its cytotoxic effects are related to its antimitotic activities, wherein apoptosis of cancer cells is induced following prolonged and irreversible mitotic blockade (Kuznetsov, G. et al, Cancer Research. 2004, 64 (16): 5760-6; Towle, M. J, et al, Cancer Research. 2010, 71 (2): 496-505). In addition to its cytotoxic, antimitotic-based mechanisms, preclinical studies in human breast cancer models have shown that eribulin also exerts complex effects on the biology of surviving cancer cells and residual tumors that appear unrelated to its antimitotic effects. Eribulin has been approved by US FDA for the treatment of metastatic breast cancer who have received at least two prior chemotherapy regimens for late-stage disease, including both anthracycline- and taxane-based chemotherapies, as well as for the treatment of liposarcoma (a specific type of soft tissue sarcoma) that cannot be removed by surgery (unresectable) or is advanced (metastatic). Eribulin has been used as payload for ADC conjugates (US20170252458). Examples of the structures of the conjugate of the antibody-Eribulins via the bridge linker are preferred as the following structures of Eb01, and Eb02.

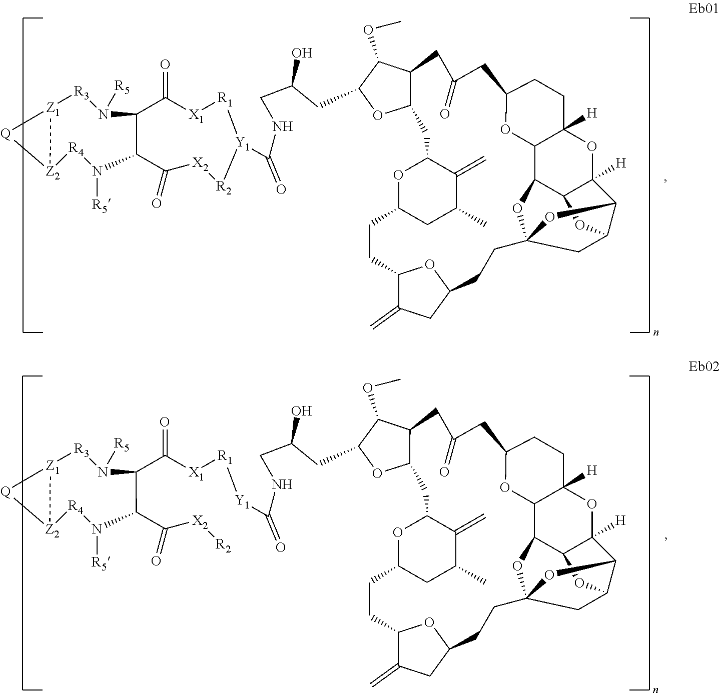

wherein “-----”, Q, X₁, X₂, Y₁, R₁, R₂, R₃, R₄, R₅, R_{5′}, Z₁, Z₂, and n are defined the same above; Preferably X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, CH₂, C(O)NHNHC(O), C(O)NR₁ or absent.

Inhibitors of nicotinamide phosphoribosyltransferase (NAMPT) are interesting ADC payloads due to their unique mechanisms of high potent activity (Sampath D, et al, Pharmacol Ther 2015; 151, 16-31). NAMPT regulates nicotinamide adenine dinucleotide (NAD) levels in cells wherein NAD plays as an essential redox cofactor to support energy and anabolic metabolism. NAD has several essential roles in metabolism. It acts as a coenzyme in redox reactions, as a donor of ADP-ribose moieties in ADP-ribosylation reactions, as a precursor of the second messenger molecule cyclic ADP-ribose, as well as acting as a substrate for bacterial DNA ligases and a group of enzymes called sirtuins that use NAD⁺ to remove acetyl groups from proteins. In addition to these metabolic functions, NAD⁺ emerges as an adenine nucleotide that can be released from cells spontaneously and by regulated mechanisms (Smyth L. M, et al, J. Biol. Chem. 2004, 279 (47), 48893-903; Billington R. A, et al, Mol Med. 2006, 12, 324-7), and can therefore have important extracellular roles (Billington R. A, et al, Mol Med. 2006, 12, 324-7). When inhibitors of NAMPT present, NAD levels decline below the level needed for metabolism resulting in energy crisis and therefore cell death. So far, clinical NAMPT inhibitor candidates FK-866, CHS-828, and GMX-1777 advanced to clinical trials but each encountered dose-limiting toxicities prior to any objective responses (Holen K., et al, Invest New Drugs 2008, 26, 45-51; Hovstadius, P., et al, Clin Cancer Res 2002, 8, 2843-50; Pishvaian, M. J., et al, J Clin Oncol 2009, 27, 3581). Thus using ADCs for targeting delivery of NAMPT inhibitors might circumvent the systemic toxicities to achieve much broader therapeutic index. Examples of the structures of the conjugate of the antibody-NAMPT inhibitors via the bridge linker are preferred as the following structures of NP01, NP02, NP03, NP04, NP05, NP06, NP07, NP08, and NP09:

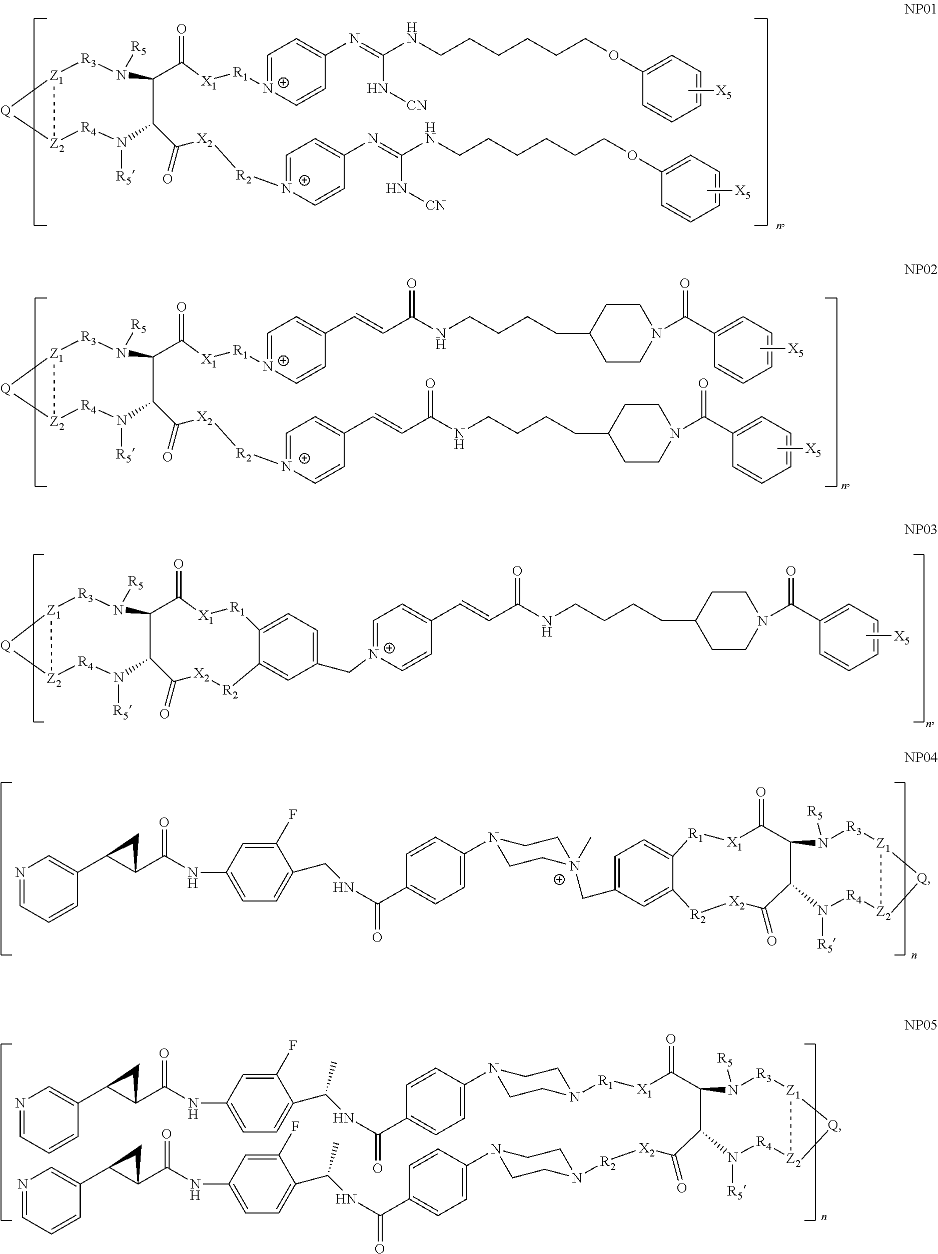

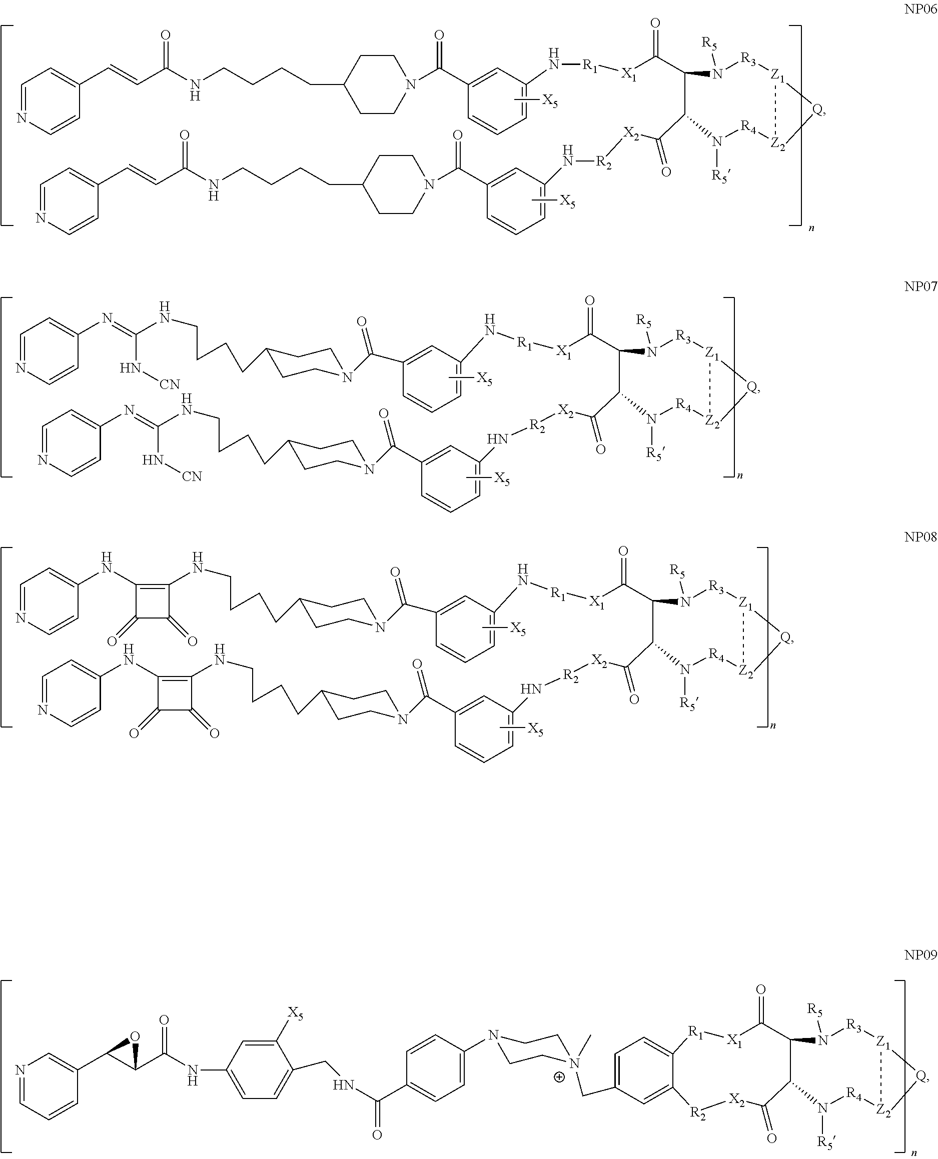

wherein “-----”, Q, X₁, X₂, Y₁, R₁, R₂, R₃, R₄, R₅, R_{5′}, Z₁, Z₂, and n are defined the same above; X₅ is F, C1, Br, I, OH, OR₁, R₁, OPO₃H₂, OSO₃H, NHR₁, OCOR₁, NHCOR₁; Preferably X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, CH₂, C(O)NHNHC(O), C(O)NR₁ or absent.

In yet another embodiment, an immunotoxin can be conjugated to a cell-binding molecule via a bis-linker of the patent. An immunotoxin herein is a macromolecular drug which is usually a cytotoxic protein derived from a bacterial or plant protein, such as Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins, etc. It also can be a highly toxic bacterial pore-forming protoxin that requires proteolytic processing for activation. An example of this protoxin is proaerolysin and its genetically modified form, topsalysin. Topsalysin is a modified recombinant protein that has been engineered to be selectively activated by an enzyme in the prostate, leading to localized cell death and tissue disruption without damaging neighboring tissue and nerves.

In yet another embodiment, cell-binding ligands or cell receptor agonists can be conjugated to a cell-binding molecule via a bis-linker of this patent. These conjugated cell-binding ligands or cell receptor agonists, in particular, antibody-receptor conjugates, can be not only to work as a targeting conductor/director to deliver the conjugate to malignant cells, but also be used to modulate or co-stimulate a desired immune response or altering signaling pathways.

In the immunotherapy, the cell-binding ligands or receptor agonists are preferred to conjugate to an antibody of TCR (T cell receptors) T cell, or of CARs (chimeric antigen receptors) T cells, or of B cell receptor (BCR), Natural killer (NK) cells, or the cytotoxic cells. Such antibody is preferably anti-CD3, CD4, CD8, CD16 (FcγRIII), CD27, CD40, CD40L, CD45RA, CD45RO, CD56, CD57, CD57bright, TNFβ, Fas ligand, MHC class I molecules (HLA-A, B, C), or NKR-P1. The cell-binding ligands or receptor agonists are selected, but not limited, from: Folate derivatives (binding to the folate receptor, a protein over-expressed in ovarian cancer and in other malignancies) (Low, P. S. et al 2008, Acc. Chem. Res. 41, 120-9); Glutamic acid urea derivatives (binding to the prostate specific membrane antigen, a surface marker of prostate cancer cells) (Hillier, S. M. et al, 2009, Cancer Res. 69, 6932-40); Somatostatin (also known as growth hormone-inhibiting hormone (GHIH) or somatotropin release-inhibiting factor (SRIF)) or somatotropin release-inhibiting hormone) and its analogues such as octreotide (Sandostatin) and lanreotide (Somatuline) (particularly for neuroendocrine tumors, GH-producing pituitary adenoma, paraganglioma, nonfunctioning pituitary adenoma, pheochromocytomas) (Ginj, M., et al, 2006, Proc. Natl. Acad. Sci. U.S.A. 103, 16436-41). In general, Somatostatin and its receptor subtypes (sst1, sst2, sst3, sst4, and sst5) have been found in many types of tumors, such as neuroendocrine tumors, in particular in GH-secreting pituitaryadenomas (Reubi J. C., Landolt, A. M. 1984 J. Clin. Endocrinol Metab 59: 1148-51; Reubi J. C., Landolt A. M. 1987 J Clin Endocrinol Metab 65: 65-73; Moyse E, et al, J Clin Endocrinol Metab 61: 98-103) and gastroenteropancreatic tumors (Reubi J. C., et al, 1987 J Clin Endocrinol Metab 65: 1127-34; Reubi, J. C, et al, 1990 Cancer Res 50: 5969-77), pheochromocytomas (Epel-baum J, et al 1995 J Clin Endocrinol Metab 80:1837-44; Reubi J. C., et al, 1992 J Clin Endocrinol Metab 74: 1082-9), neuroblastomas (Prevost G, 1996 Neuroendocrinology 63:188-197; Moertel, C. L, et al 1994 Am J Clin Path 102:752-756), medullary thyroid cancers (Reubi, J. C, et al 1991 Lab Invest 64:567-573) small cell lung cancers (Sagman U, et al, 1990 Cancer 66:2129-2133), nonneuroendocrine tumors including brain tumors such as meningiomas, medulloblastomas, or gliomas (Reubi J. C., et al 1986 J Clin Endocrinol Metab 63: 433-8; Reubi J. C., et al 1987 Cancer Res 47: 5758-64; Fruhwald, M. C, et al 1999 Pediatr Res 45: 697-708), breast carcinomas (Reubi J. C., et al 1990 Int J Cancer 46: 416-20; Srkalovic G, et al 1990 J Clin Endocrinol Metab 70: 661-669), lymphomas (Reubi J. C., et al 1992, Int J Cancer 50: 895-900), renal cell cancers (Reubi J. C., et al 1992, Cancer Res 52: 6074-6078), mesenchymal tumors (Reubi J. C., et al 1996 Cancer Res 56: 1922-31), prostatic (Reubi J. C., et al 1995, J. Clin. Endocrinol Metab 80: 2806-14; et al 1989, Prostate 14:191-208; Halmos G, et al J. Clin. Endocrinol Metab 85: 2564-71), ovarian (Halmos, G, et al, 2000 J Clin Endocrinol Metab 85: 3509-12; Reubi J. C., et al 1991 Am J Pathol 138:1267-72), gastric (Reubi J. C., et al 1999, Int J Cancer 81: 376-86; Miller, G. V, 1992 Br J Cancer 66: 391-95), hepatocellular (Kouroumalis E, et al 1998 Gut 42: 442-7; Reubi J. C., et al 1999 Gut 45: 66-774) and nasopharyngeal carcinomas (Loh K. S, et al, 2002 Virchows Arch 441: 444-8); certain Aromatic sulfonamides, specific to carbonic anhydrase IX (a marker of hypoxia and of renal cell carcinoma) (Neri, D., et al, Nat. Rev. Drug Discov. 2011, 10, 767-7); Pituitary adenylate cyclase activating peptides (PACAP) (PAC1) for pheochromocytomas and paragangliomas; Vasoactive intestinal peptides (VIP) and their receptor subtypes (VPAC1, VPAC2) for cancers of lung, stomach, colon, rectum, breast, prostate, pancreatic ducts, liver, urinary bladder and epithelial tumors; α-Melanocyte-stimulating hormone (α-MSH) receptors for various tumors; Cholecystokinin (CCK)/gastrin receptors and their receptor subtypes (CCK1 (formerly CCK-A) and CCK2 for small cell lung cancers, medullary thyroid carcinomas, astrocytomas, insulinomas and ovarian cancers; Bombesin(Pyr-Gln-Arg-Leu-Gly-Asn-Gln-Trp-Ala-Val-Gly-His-Leu-Met-NH₂)/gastrin-releasing peptide (GRP) and their receptor subtypes (BB1, GRP receptor subtype (BB2), the BB3 and BB4) for renal cell, breast, lung, gastric and prostate carcinomas, and neuroblastoma (and neuroblastoma (Ohlsson, B., et al, 1999, Scand. J. Gastroenterology 34 (12): 1224-9; Weber, H. C., 2009, Cur. Opin. Endocri. Diab. Obesity 16(1): 66-71, Gonzalez N, et al, 2008, Cur. Opin. Endocri. Diab. Obesity 15(1), 58-64); Neurotensin receptors and its receptor subtypes (NTR1, NTR2, NTR3) for small cell lung cancer, neuroblastoma, pancreatic, colonic cancer and Ewing sarcoma; Substance P receptors and their receptor subtypes (such as NK1 receptor for Glial tumors, Hennig I. M., et al 1995 Int. J. Cancer 61, 786-792); Neuropeptide Y (NPY) receptors and its receptor subtypes (Y1-Y6) for breast carcinomas; Homing Peptides include RGD (Arg-Gly-Asp), NGR (Asn-Gly-Arg), the dimeric and multimeric cyclic RGD peptides (e.g. cRGDfV) that recognize receptors (integrins) on tumor surfaces (Laakkonen P, Vuorinen K. 2010, Integr Biol (Camb). 2(7-8): 326-337; Chen K, Chen X. 2011, Theranostics. 1:189-200; Garanger E, et al, Anti-Cancer Agents Med Chem. 7 (5): 552-558; Kerr, J. S. et al, Anticancer Research, 19(2A), 959-968; Thumshirn, G, et al, 2003 Chem. Eur. J. 9, 2717-2725), and TAASGVRSMH or LTLRWVGLMS (chondroitin sulfate proteoglycan NG2 receptor) and F3 peptides (31 amino acid peptide that binds to cell surface-expressed nucleolin receptor) (Zitzmann, S., 2002 Cancer Res., 62, 18, pp. 5139-5143, Temminga, K., 2005, Drug Resistance Updates, 8, 381-402; P. Laakkonen and K. Vuorinen, 2010 Integrative Biol, 2(7-8), 326-337; M. A. Burg, 1999 Cancer Res., 59(12), 2869-2874; K. Porkka, et al 2002, Proc. Nat. Acad. Sci. USA 99(11), 7444-9); Cell Penetrating Peptides (CPPs) (Nakase I, et al, 2012, J. Control Release. 159(2), 181-188); Peptide Hormones, such as luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, e.g. buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloro-Phe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂) (Thundimadathil, J., J. Amino Acids, 2012, 967347, doi:10.1155/2012/967347; Boccon-Gibod, L.; et al, 2011, Therapeutic Advances in Urology 3(3): 127-140; Debruyne, F., 2006, Future Oncology, 2(6), 677-696; Schally A. V; Nagy, A. 1999 Eur J Endocrinol 141:1-14; Koppan M, et al 1999 Prostate 38:151-158); and Pattern Recognition Receptors (PRRs), such as Toll-like receptors (TLRs), C-type lectins and Nodlike Receptors (NLRs) (Fukata, M., et al, 2009, Semin. Immunol. 21, 242-253; Maisonneuve, C., et al, 2014, Proc. Natl. Acad. Sci. U.S.A 111, 1-6; Botos, I., et al, 2011, Structure 19, 447-459; Means, T. K., et al, 2000, Life Sci. 68, 241-258) that range in size from small molecules (imiquimod, guanisine and adenosine analogs) tolarge and complex biomacromolecules such as lipopolysaccharide (LPS), nucleic acids (CpG DNA, polyL:C) and lipopeptides (Pam3CSK4) (Kasturi, S. P., et al, 2011, Nature 470, 543-547; Lane, T., 2001, J. R. Soc. Med. 94, 316; Hotz, C., and Bourquin, C., 2012, Oncoimmunology 1, 227-228; Dudek, A. Z., et al, 2007, Clin. Cancer Res. 13, 7119-25); Calcitonin receptors which is a 32-amino-acid neuropeptide involved in the regulation of calcium levels largely through its effects on osteoclasts and on the kidney (Zaidi M, et al, 1990 Crit Rev Clin Lab Sci 28, 109-174; Gorn, A. H., et al 1995 J Clin Invest 95:2680-91); And integrin receptors and their receptor subtypes (such as α_{V}β₁, α_{V}β₃, α_{V}β₅, α_{V}β₆, α₆β₄, α₇β₁, α_{L}β₂, α_{IIb}β₃, etc.) which generally play important roles in angiogenesis are expressed on the surfaces of a variety of cells, in particular, of osteoclasts, endothelial cells and tumor cells (Ruoslahti, E. et al, 1994 Cell 77, 477-8; Albelda, S. M. et al, 1990 Cancer Res., 50, 6757-64). Short peptides, GRGDSPK and Cyclic RGD pentapeptides, such as cyclo(RGDfV) (L1) and its derives [cyclo(-N(Me)R-GDfV), cyclo(R-Sar-DfV), cyclo-(RG-N(Me)D-fV), cyclo(RGD-N(Me)f-V), cyclo(RGDf-N(Me)V-)(Cilengitide)] have shown high binding affinities of the intergrin receptors (Dechantsreiter, M. A. et al, 1999 J. Med. Chem. 42, 3033-40, Goodman, S. L., et al, 2002 J. Med. Chem. 45, 1045-51).

The cell-binding ligands or cell receptor agonists can be Ig-based and non-Ig-based protein scaffold molecules. The Ig-Based scaffolds can be selected, but not limited, from Nanobody (a derivative of VHH (camelid Ig)) (Muyldermans S., 2013 Annu Rev Biochem. 82, 775-97); Domain antibodies (dAb, a derivative of VH or VL domain) (Holt, L. J, et al, 2003, Trends Biotechnol. 21, 484-90); Bispecific T cell Engager (BiTE, a bispecific diabody) (Baeuerle, P. A, et al, 2009, Curr. Opin. Mol. Ther. 11, 22-30); Dual Affinity ReTargeting (DART, a bispecific diabody) (Moore P. A. P, et al. 2011, Blood 117(17), 4542-51); Tetravalent tandem antibodies (TandAb, a dimerized bispecific diabody) (Cochlovius, B, et al. 2000, Cancer Res. 60(16):4336-4341). The Non-Ig scaffolds can be selected, but not limited, from Anticalin (a derivative of Lipocalins) (Skerra A. 2008, FEBS J., 275(11): 2677-83; Beste G, et al, 1999 Proc. Nat. Acad. USA. 96(5):1898-903; Skerra, A. 2000 Biochim Biophys Acta, 1482(1-2): 337-50; Skerra, A. 2007, Curr Opin Biotechnol. 18(4): 295-304; Skerra, A. 2008, FEBS J. 275(11):2677-83); Adnectins (10th FN3 (Fibronectin)) (Koide, A, et al, 1998 J. Mol. Biol., 284(4):1141-51; Batori V, 2002, Protein Eng. 15(12): 1015-20; Tolcher, A. W, 2011, Clin. Cancer Res. 17(2): 363-71; Hackel, B. J, 2010, Protein Eng. Des. Sel. 23(4): 211-19); Designed Ankyrin Repeat Proteins (DARPins) (a derivative of ankrin repeat (AR) proteins) (Boersma, Y. L, et al, 2011 Curr Opin Biotechnol. 22(6): 849-57), e.g. DARPin C9, DARPin Ec4 and DARPin E69_LZ3_E01 (Winkler J, et al, 2009 Mol Cancer Ther. 8(9), 2674-83; Patricia M-K. M., et al, Clin Cancer Res. 2011; 17(1):100-10; Boersma Y. L, et al, 2011 J. Biol. Chem. 286(48), 41273-85); Avimers (a domain A/low-density lipoprotein (LDL) receptor) (Boersma Y. L, 2011 J. Biol. Chem. 286(48): 41273-41285; Silverman J, et al, 2005 Nat. Biotechnol., 23(12):1556-61).

Examples of the structures of the conjugate of the antibody-cell-binding ligands or cell receptor agonists or drugs via the bis-linker of the patent application are listed as the following: LB01 (Folate conjugate), LB02 (PMSA ligand conjugate), LB03 (PMSA ligand conjugate), LB04 (PMSA ligand conjugate), LB05 (Somatostatin conjugate), LB06 (Somatostatin conjugate), LB07 (Octreotide, a Somatostatin analog conjugate), LB08 (Lanreodde, a Somatostatin analog conjugate), LB09 (Vapreotide (Sanvar), a Somatostatin analog conjugate), LB10 (CAIX ligand conjugate), LB11 (CAIX ligand conjugate), LB12 (Gastrin releasing peptide receptor (GRPr), MBA conjugate), LB13 (luteinizing hormone-releasing hormone (LH-RH) ligand and GnRH conjugate), LB14 (luteinizing hormone-releasing hormone (LH-RH) and GnRH ligand conjugate), LB15 (GnRH antagonist, Abarelix conjugate), LB16 (cobalamin, vitamin B12 analog conjugate), LB17 (cobalamin, vitamin B12 analog conjugate), LB18 (for αᵥβ₃ integrin receptor, cyclic RGD pentapeptide conjugate), LB19 (hetero-bivalent peptide ligand conjugate for VEGF receptor), LB20 (Neuromedin B conjugate), LB21 (bombesin conjugate for a G-protein coupled receptor), LB22 (TLR₂ conjugate for a Toll-like receptor), LB23 (for an androgen receptor), LB24 (Cilengitide/cyclo(-RGDfV-) conjugate for an αᵥ intergrin receptor, LB23 (Fludrocortisone conjugate), LB25 (Rifabutin analog conjugate), LB26 (Rifabutin analog conjugate), LB27 (Rifabutin analog conjugate), LB28 (Fludrocortisone conjugate), LB29 (Dexamethasone conjugate), LB30 (fluticasone propionate conjugate), LB31 (Beclometasone dipropionate conjugate), LB32 (Triamcinolone acetonide conjugate), LB33 (Prednisone conjugate), LB34 (Prednisolone conjugate), LB35 (Methylprednisolone conjugate), LB36 (Betamethasone conjugate), LB37 (Irinotecan analog conjugate), LB38 (Crizotinib analog conjugate), LB39 (Bortezomib analog conjugate), LB40 (Carfilzomib analog conjugate), LB41 (Carfilzomib analog conjugate), LB42 (Leuprolide analog conjugate), LB43 (Triptorelin analog conjugate), LB44 (Clindamycin conjugate), LB45 (Liraglutide analog conjugate), LB46 (Semaglutide analog conjugate), LB47 (Retapamulin analog conjugate), LB48 (Indibulin analog conjugate), LB49 (Vinblastine analog conjugate), LB50 (Lixisenatide analog conjugate), LB51 (Osimertinib analog conjugate), LB52 (a neucleoside analog conjugate), LB53 (Erlotinib analog conjugate) and LB54 (Lapatinib analog conjugate) which are shown in the following structures:

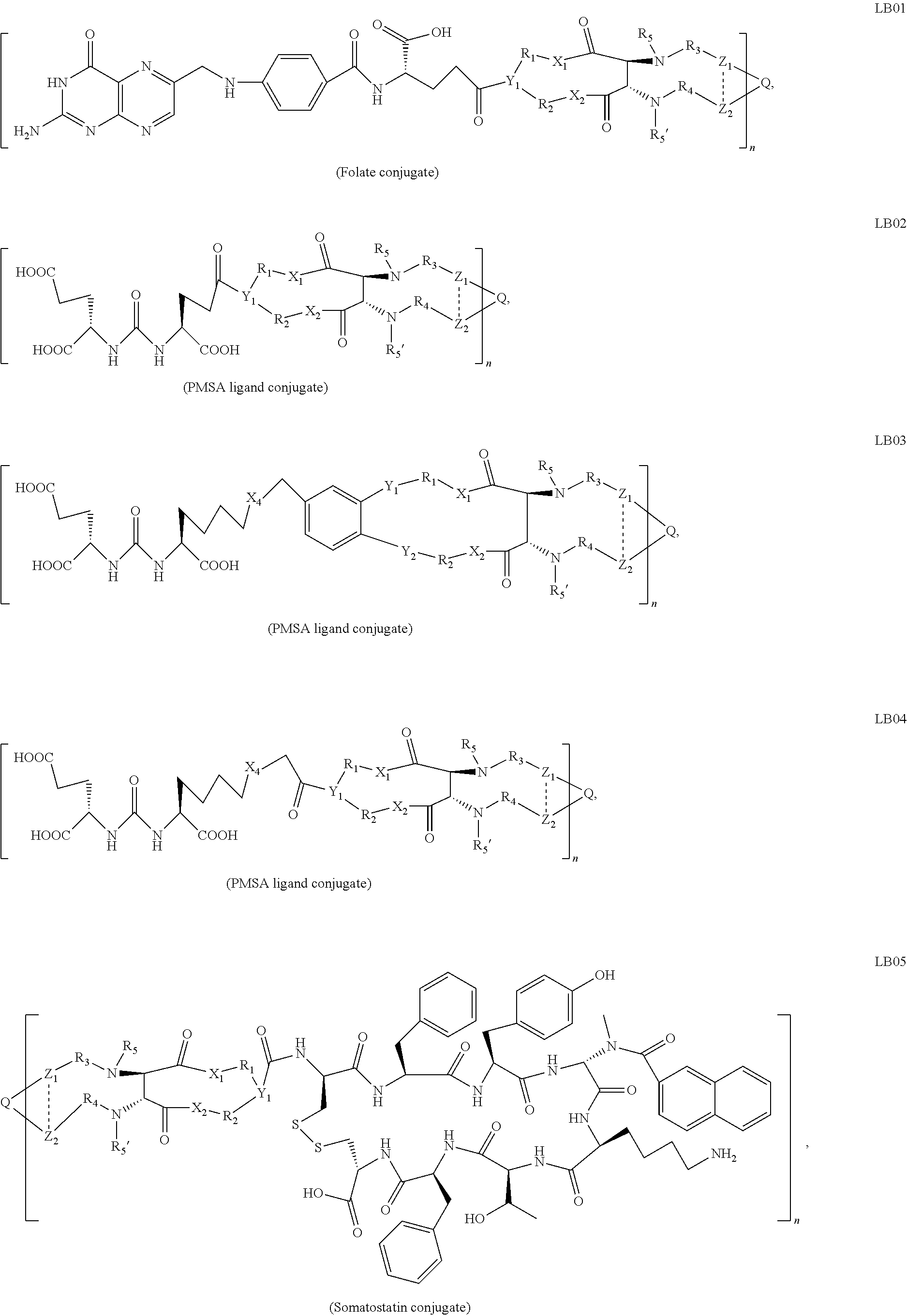

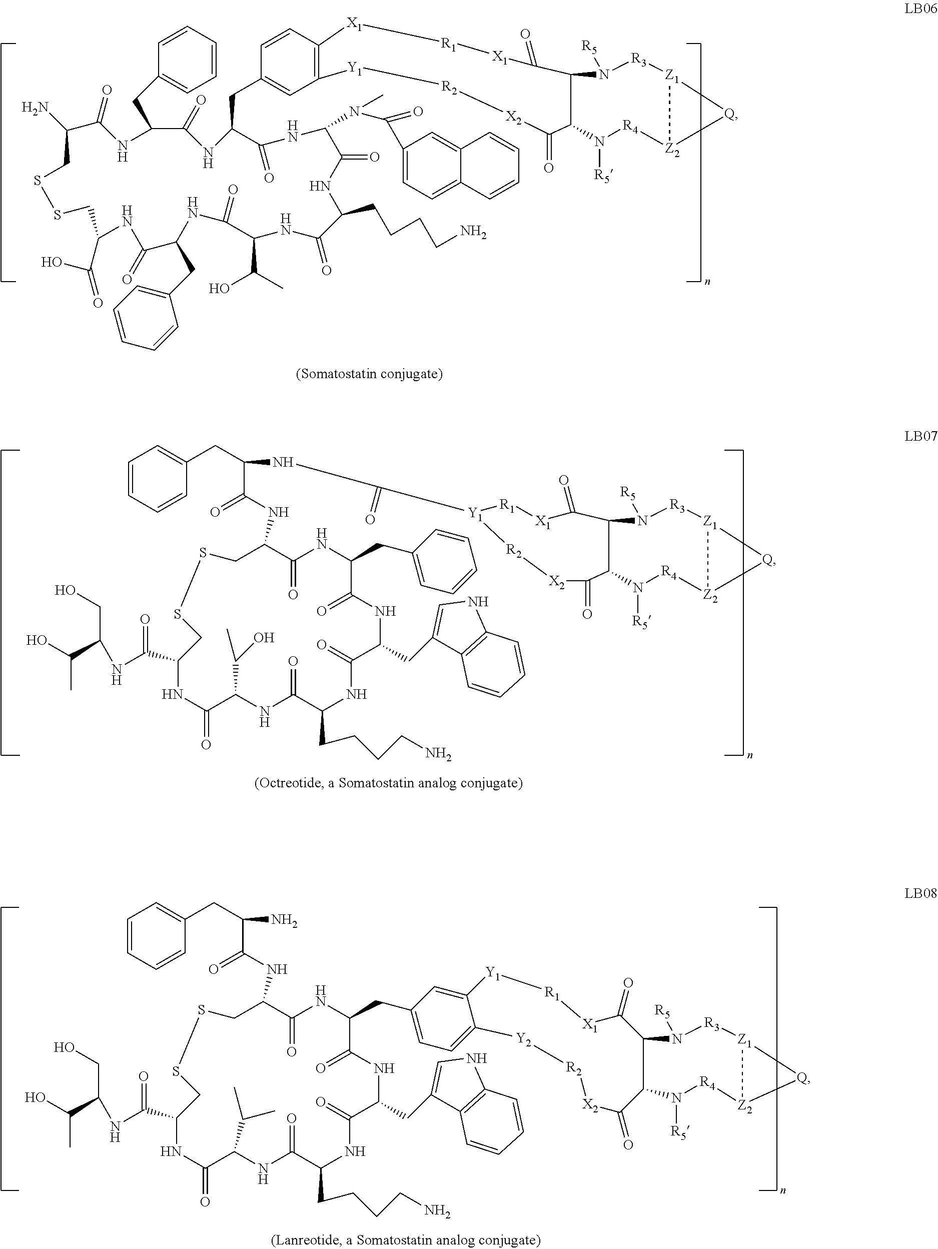

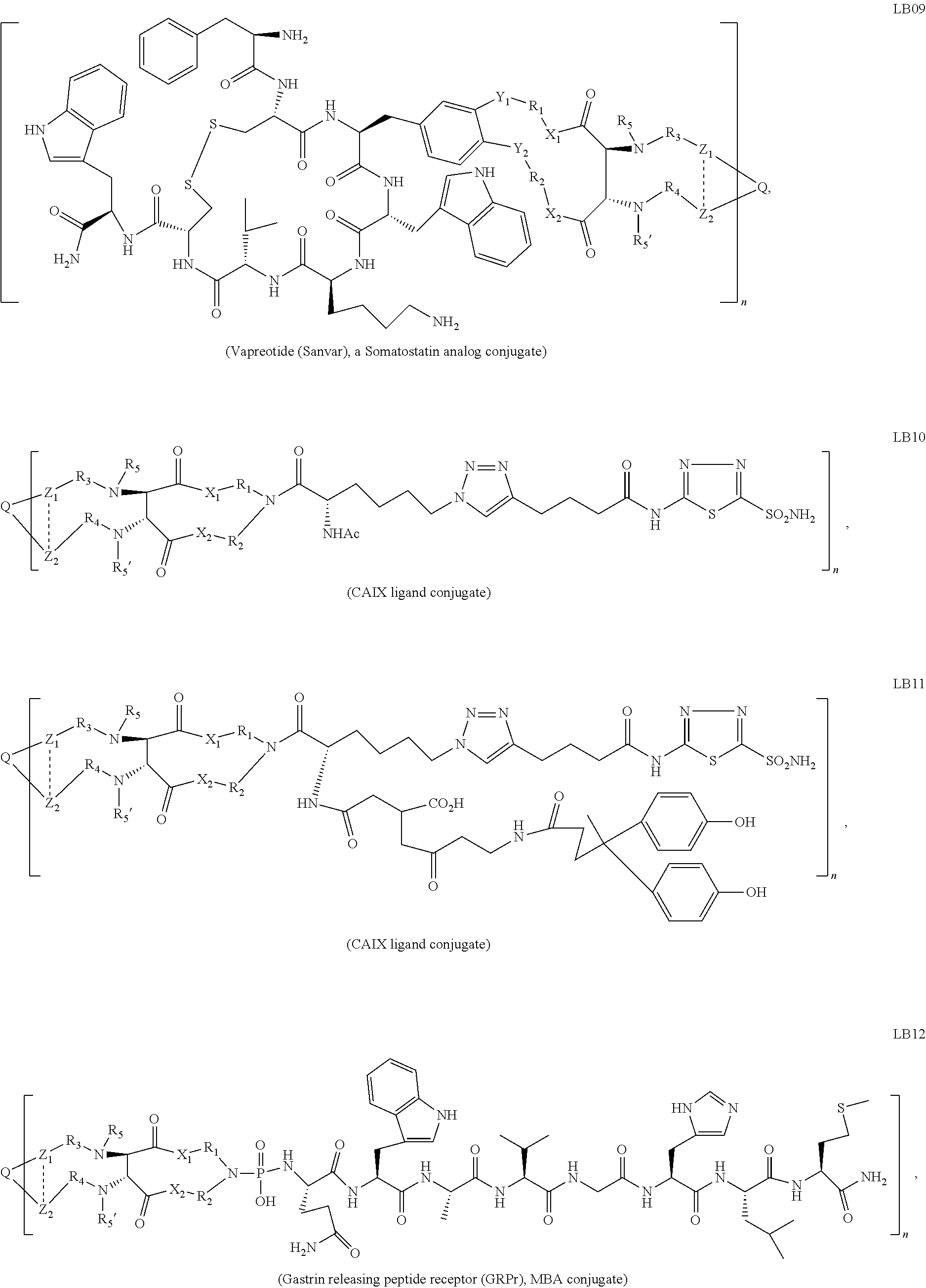

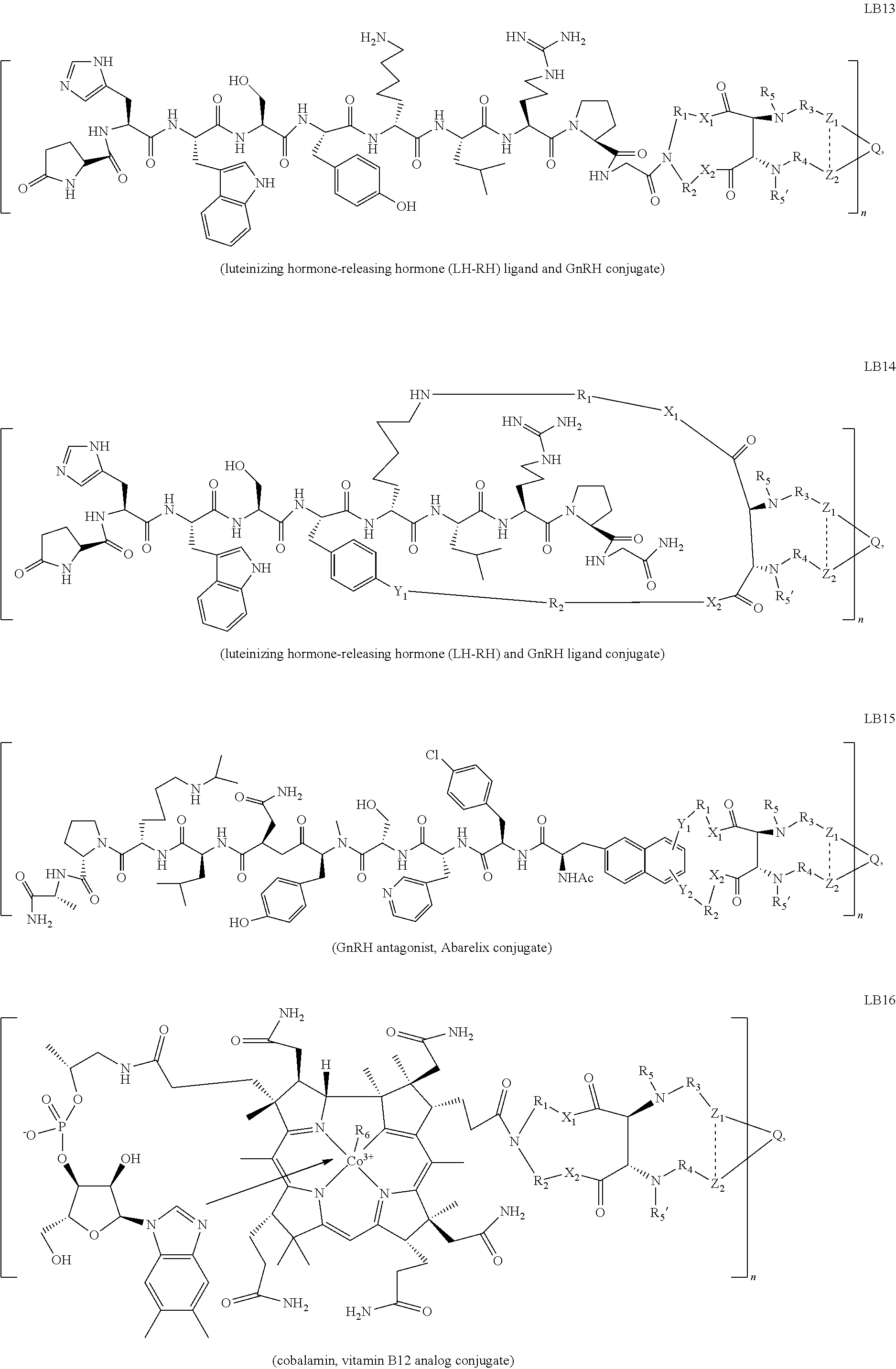

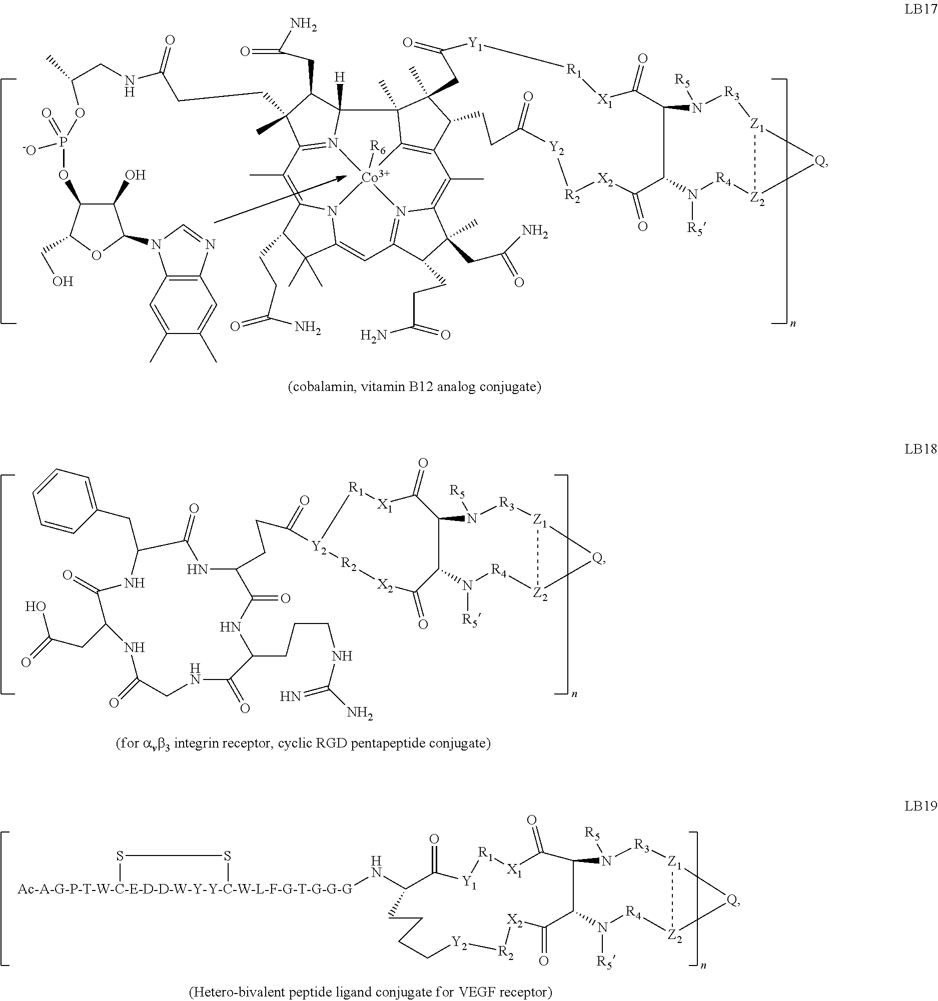

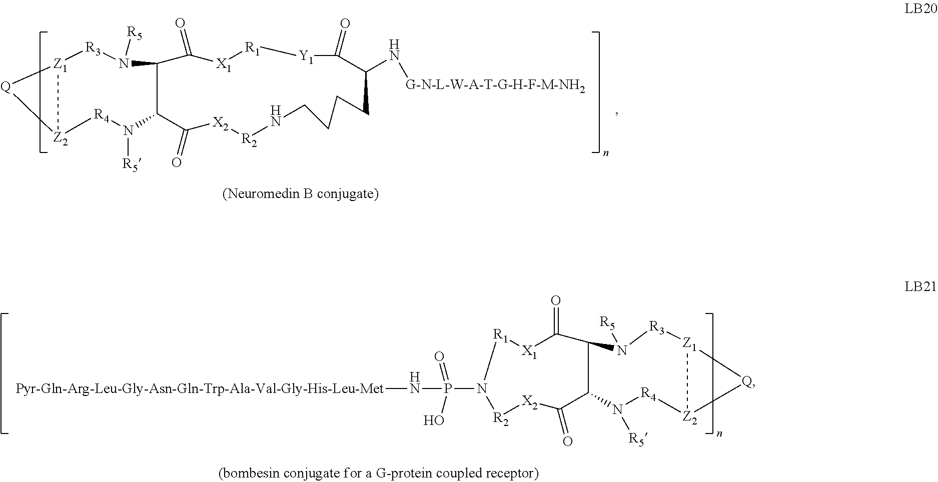

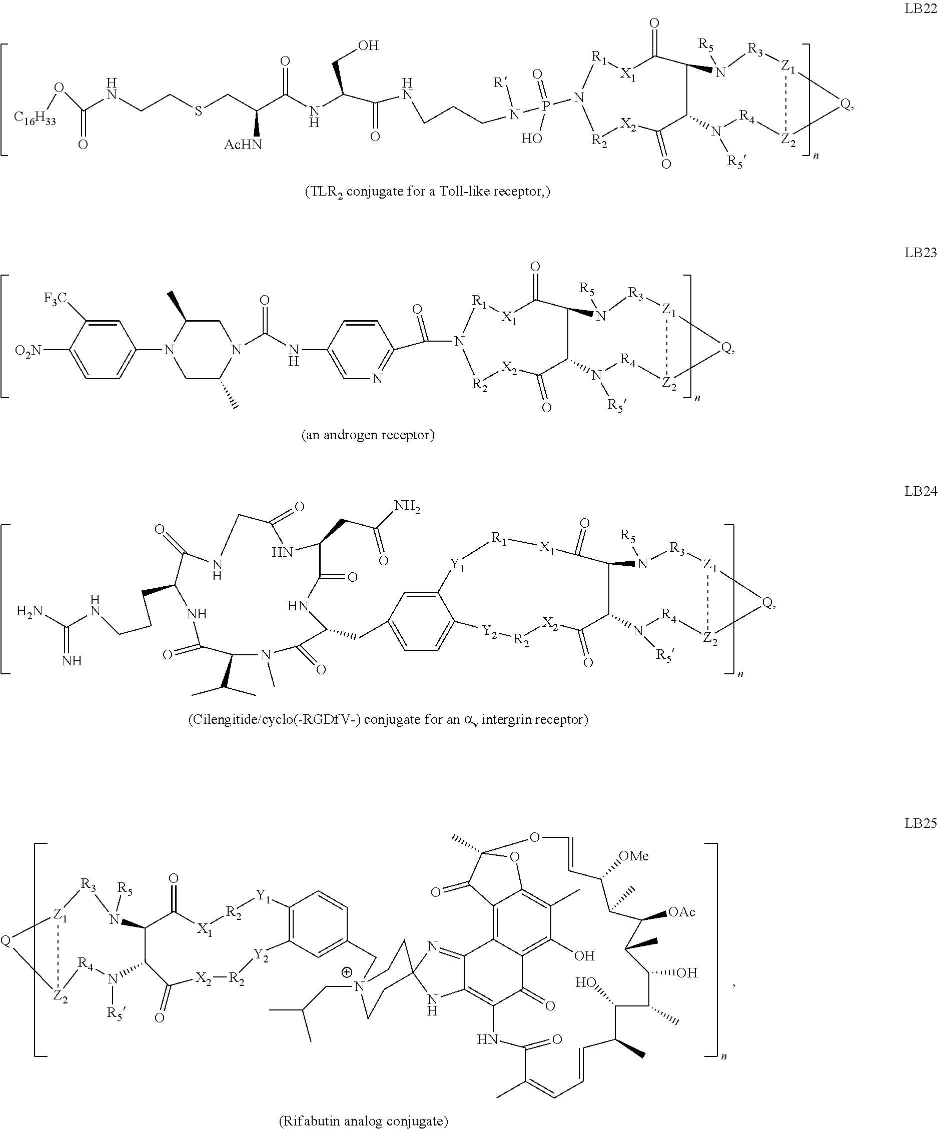

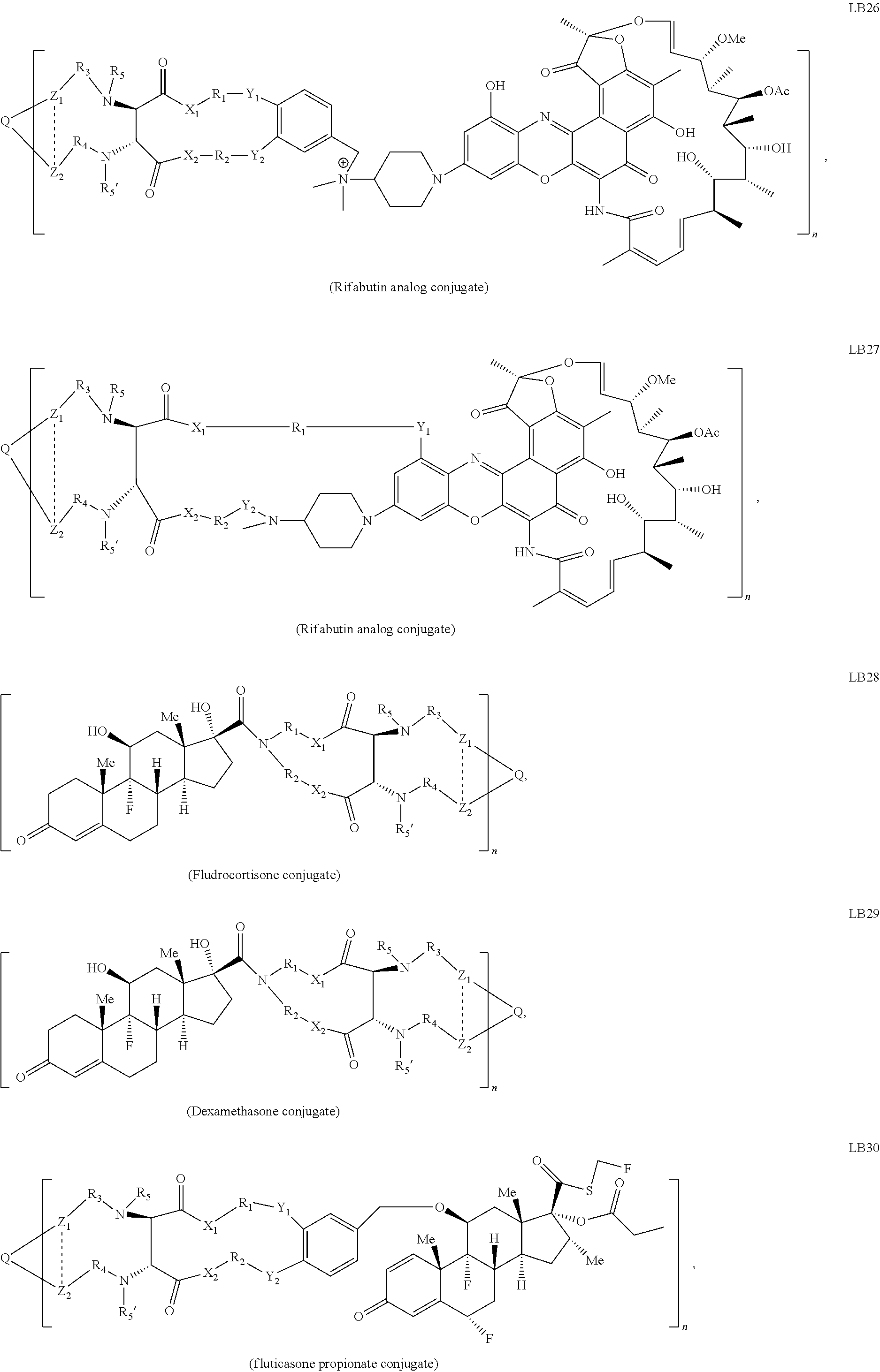

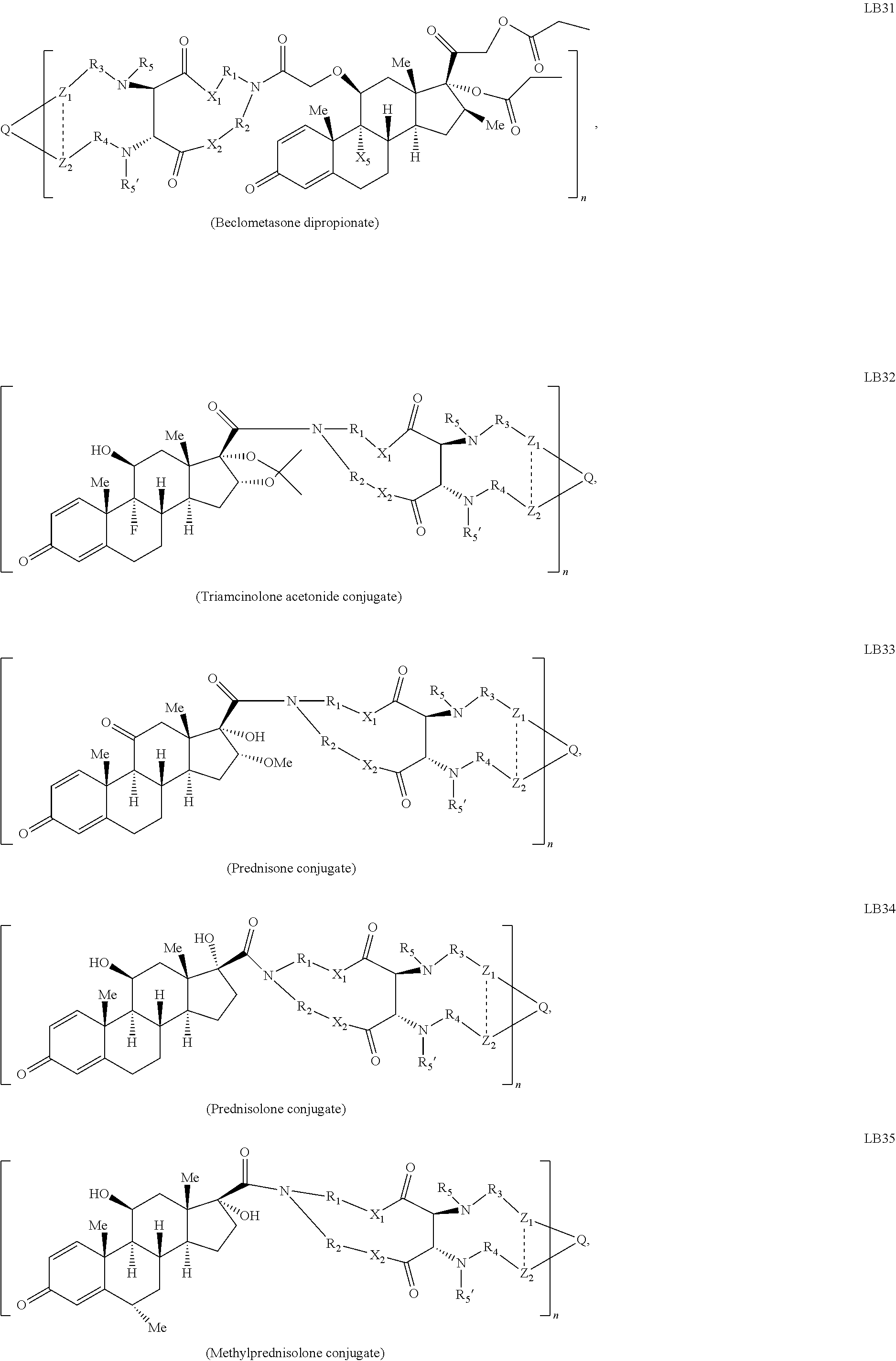

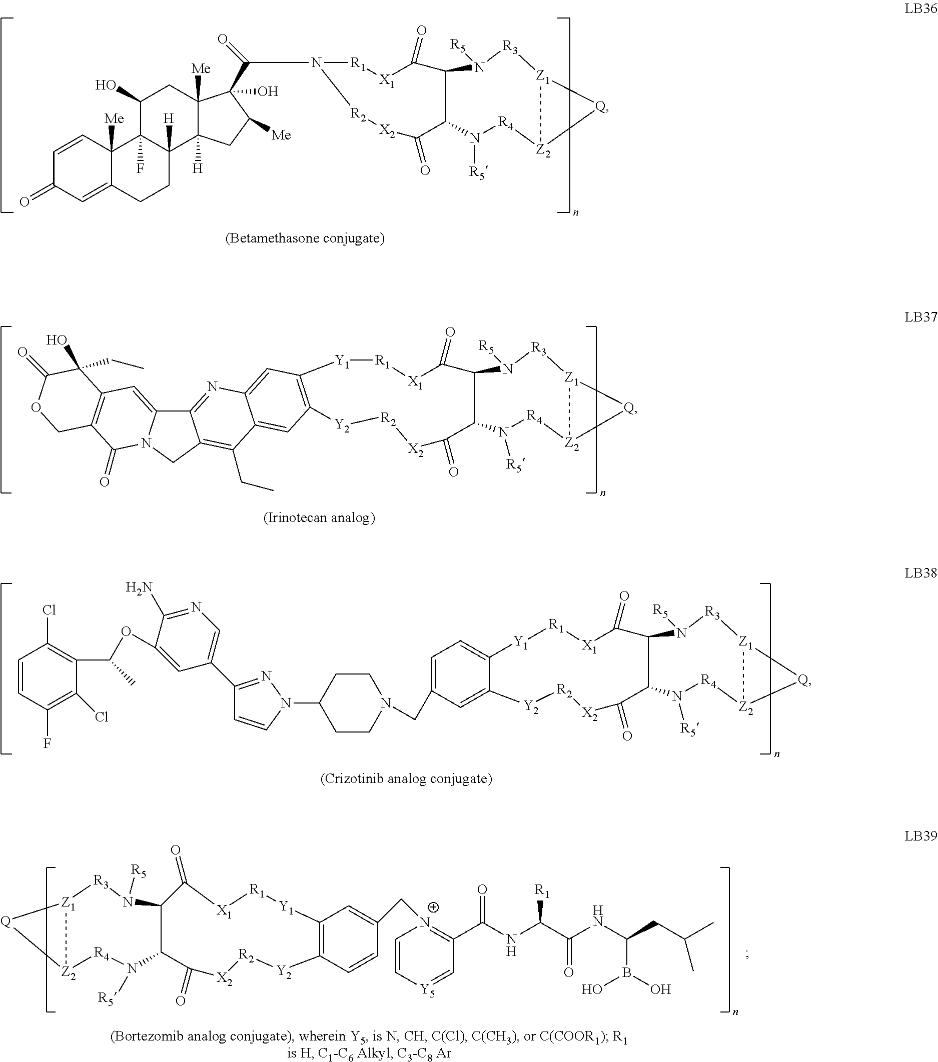

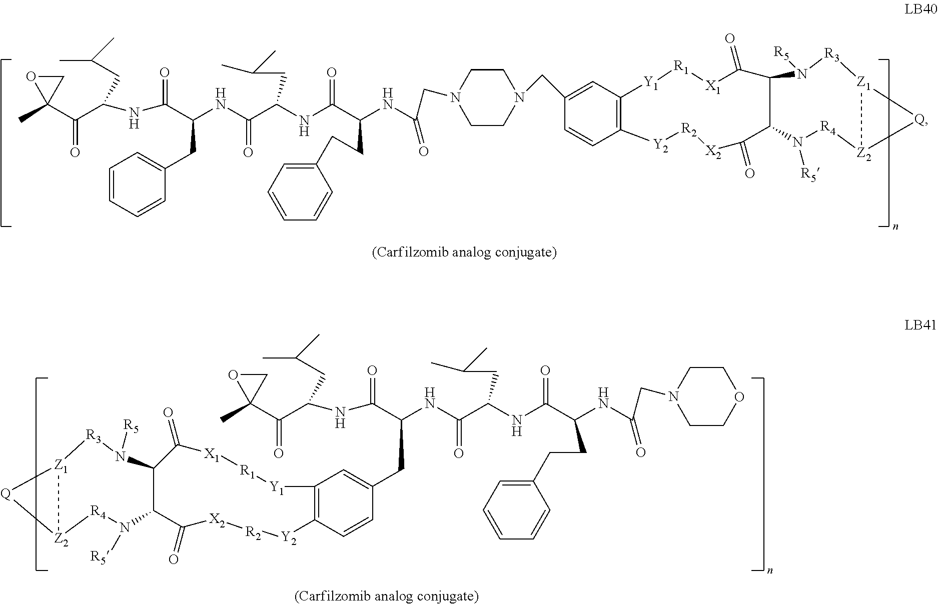

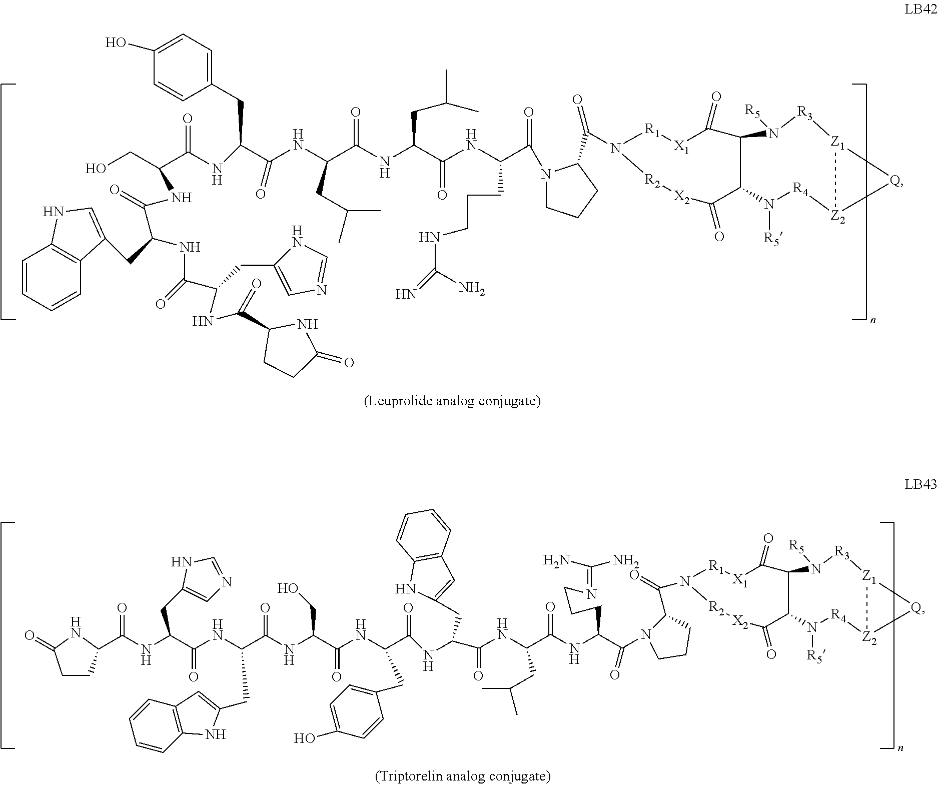

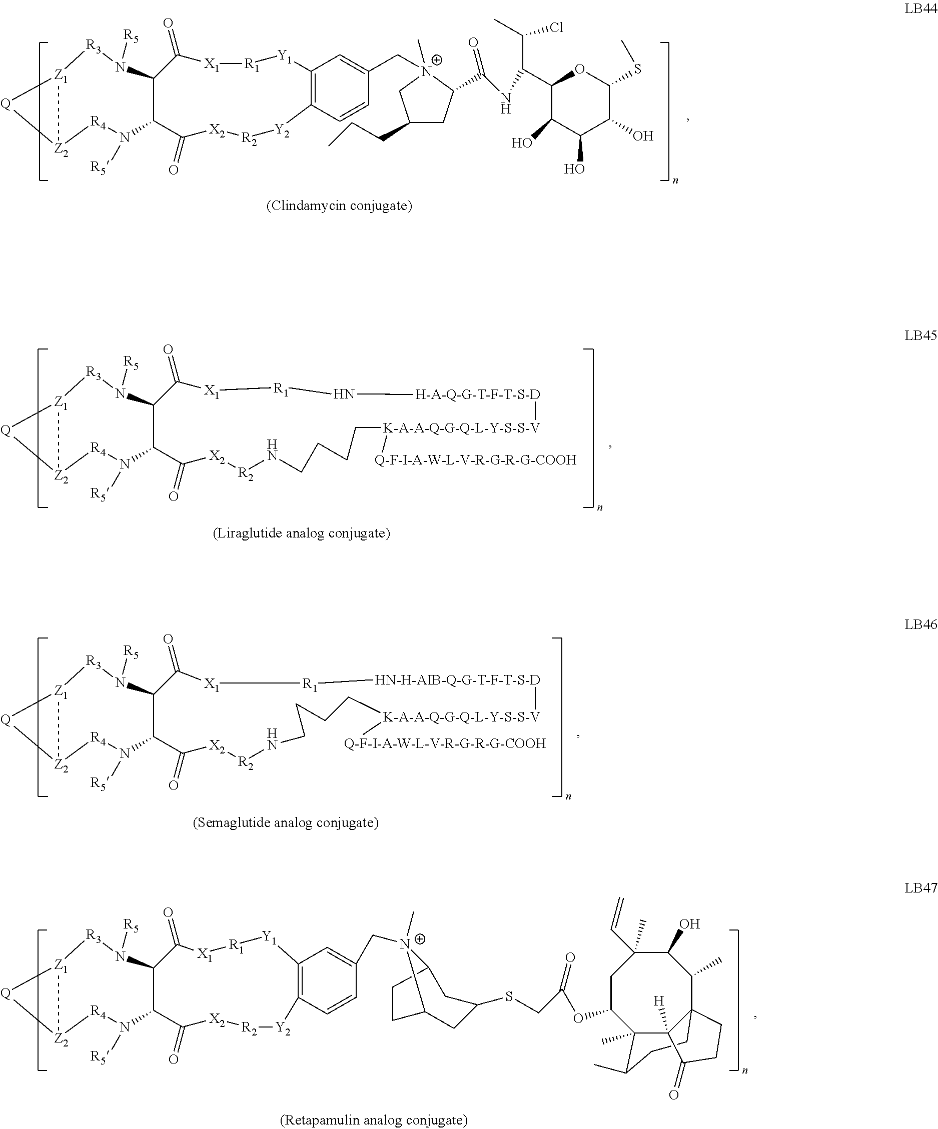

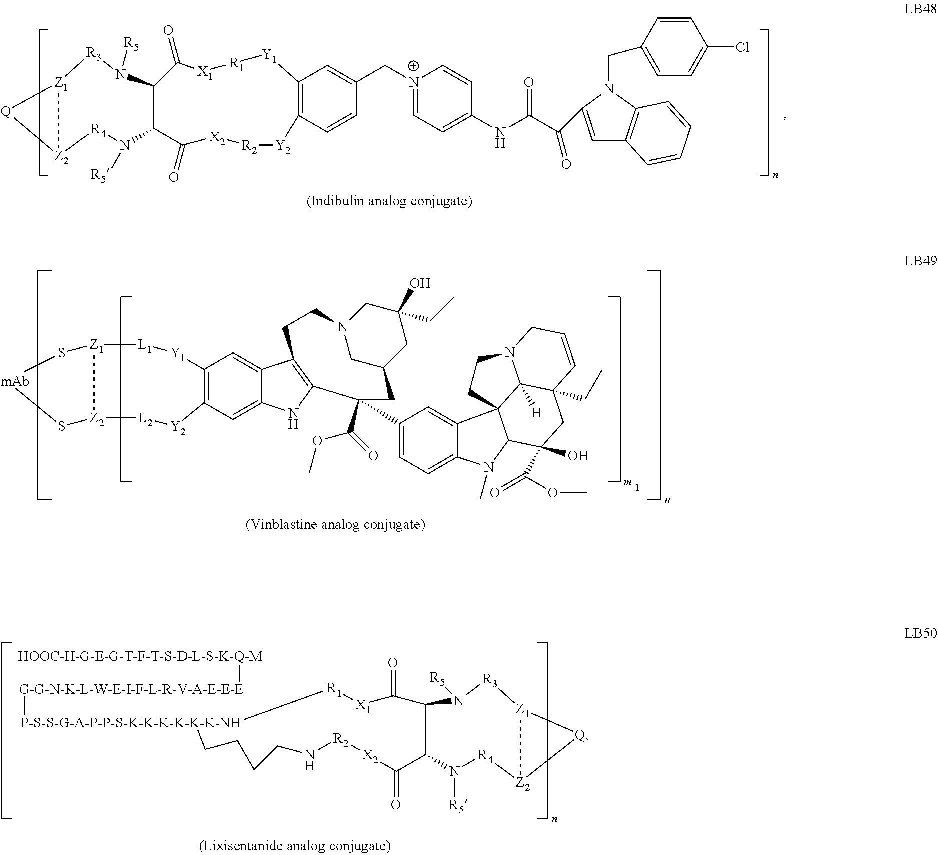

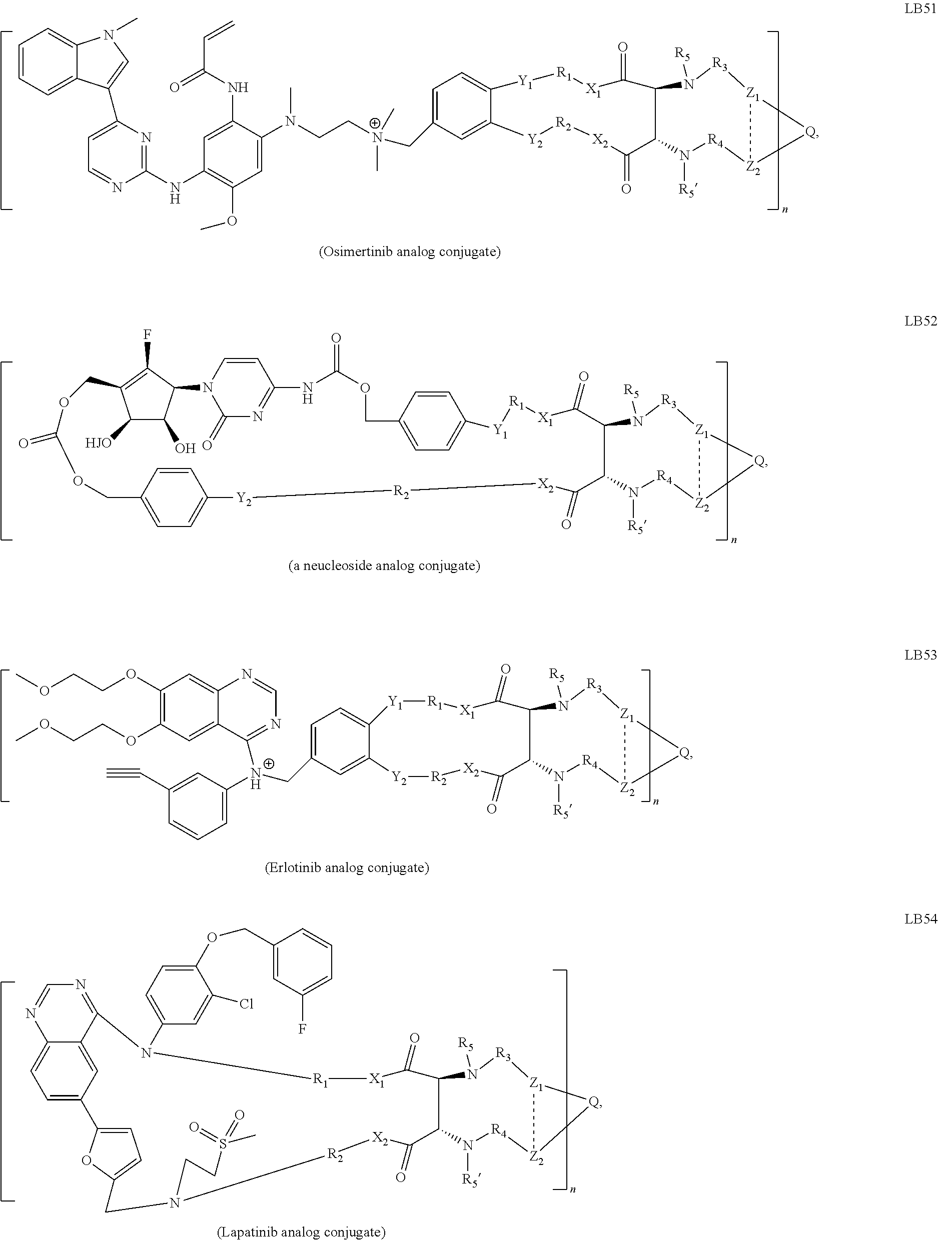

wherein “-----”, X₁, X₂, Q, Y₁, Y₂, R₁, R₂, R₃, R₄, R₅, R_{5′}, Z₁, Z₂, and n are defined the same above; Preferably X₁ X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; X₃ is CH₂, O, NH, NHC(O), NHC(O)NH, C(O), OC(O), OC(O)(NR₃), R₁, NHR₁, NR₁, C(O)R₁ or absent; X₄ is H, CH_, OH, O, C(O), C(O)NH, C(O)N(R₁), R₁, NHR₁, NR₁, C(O)R₁ or C(O)O; X₅ is H, CH₃, F, or Cl; M₁ and M₂ are independently H, Na, K, Ca, Mg, NH₄, NR₁R₂R₃; R₆ is 5′-deoxyadenosyl, Me, OH, or CN;

In yet another embodiment, one, two or more DNA, RNA, mRNA, small interfering RNA (siRNA), microRNA (miRNA), and PIWI interacting RNAs (piRNA) are preferred conjugated to a cell-binding molecule via a bis-linker of this patent. Small RNAs (siRNA, miRNA, piRNA) and long non-coding antisense RNAs are known responsible for epigenetic changes within cells (Goodchild, J (2011), Methods in molecular biology (Clifton, N.J.). 764: 1-15). DNA, RNA, mRNA, siRNA, miRNA or piRNA herein can be single or double strands with nucleotide units from 3 to 10 million and some of their nucleotide can be none natural (synthetic) forms, such as oligonucleotide with phosphorothioate linkage as example of Fomivirsen, or the nucleotides are linked with phosphorothioate linkages rather than the phosphodiester linkages of natural RNA and DNA, and the sugar parts are deoxyribose in the middle part of the molecule and 2′-O-methoxyethyl-modified ribose at the two ends as example Mipomersen, or oligonucleotide made with peptide nucleic acid (PNA), Morpholino, Phosphorothioate, Thiophosphoramidate, or with 2′-O-Methoxyethyl (MOE), 2′-O-Methyl, 2′-Fluoro, Locked Nucleic Acid (LNA), or Bicyclic Nucleic Acid (BNA) of ribose sugar, or nucleic acids are modified to remove the 2′-3′ carbon bond in the sugar ring (Whitehead, K. A.; et al (2011), Annual Review of Chemical and Biomolecular Engineering 2: 77-96; Bennett, C. F.; Swayze, E. E. (2010), Annu. Rev. Pharmacol. Toxicol. 50: 259-29). Preferably, oligonucleotide range in length is from approximately 8 to over 100 nucleotides. An example of the structure of the conjugates is displayed below:

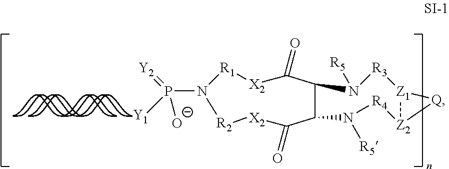

wherein “-----”, Q, Y₁, Y₂, R₁, R₂, R₃, R₄, R₅, R_{5′}, Z₁, Z₂, and n are defined the same above; Preferably X₁ X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, CH₂, C(O)NHNHC(O) and C(O)NR₁; [symbol] is single or double strands of DNA, RNA, mRNA, siRNA, miRNA, or piRNA.

In yet another embodiment, IgG antibody conjugates conjugated with one, or two, or more differently function molecules or drugs are preferred to be conjugated specifically to a pair of thiols (through reduction of the disulfide bonds) between the light chain and heavy chain, the upper disulfide bonds between the two heavy chains, and the lower disulfide bonds between the two heavy chains as shown in the following structure, ST1, ST2, ST3, ST4, ST5, or ST6:

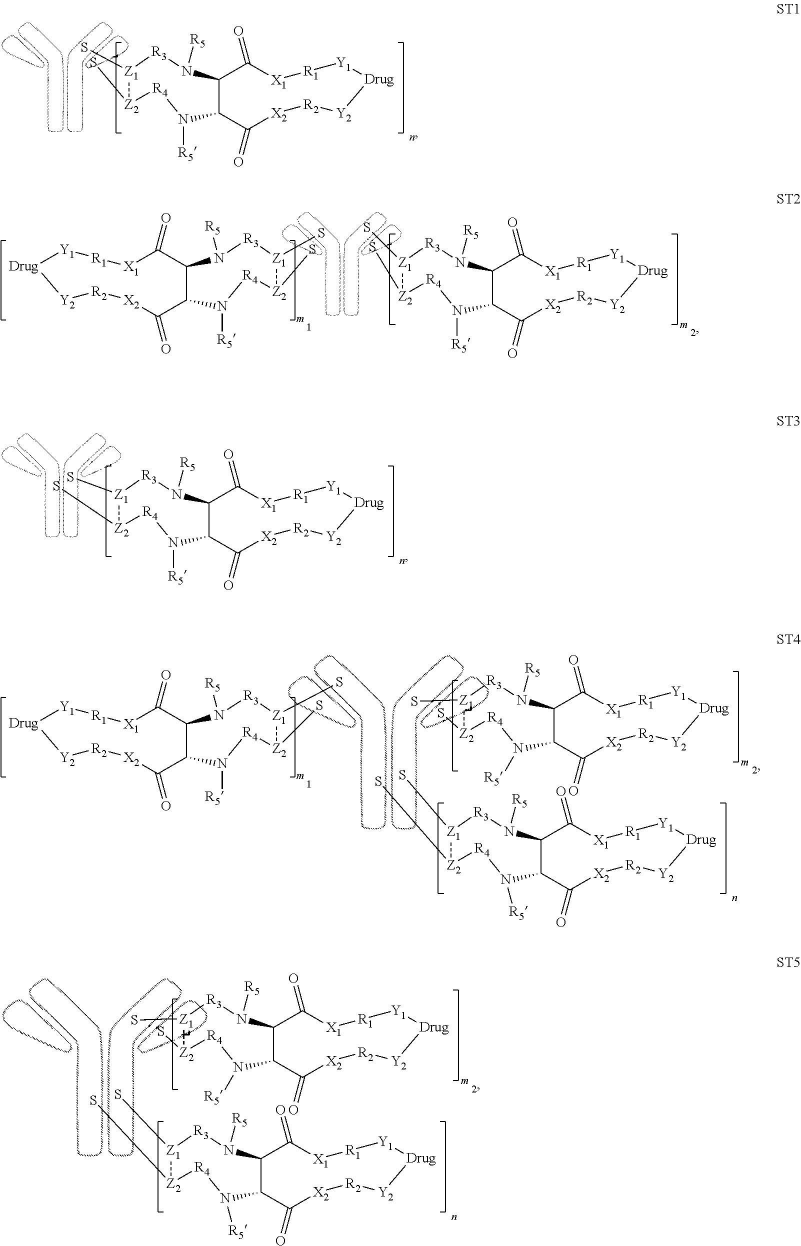

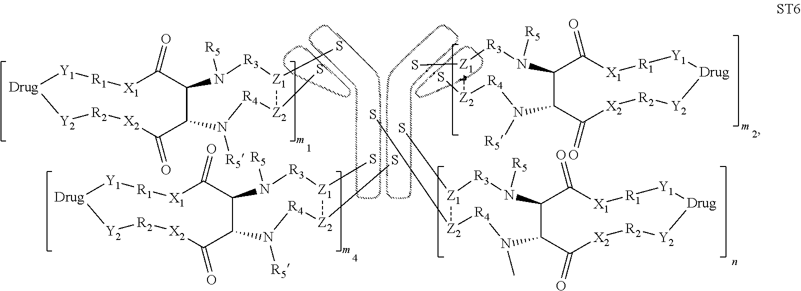

wherein “---”, Y1, Y₂, R₁, R₂, R₃, R₄, R₅, R_{5′}, Z₁, Z₂, and n are defined the same above; Preferably X₁ X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, CH₂, C(O)NHNHC(O) and C(O)NR₁; m₁, m₂, m₃, and m₄ are independently 1-30.

In addition, the drug or cytotoxic molecules Y₁, Y₂, R₁, R₂, R₃, R₄, R₅, R_{5′}, Z₁, Z₂, and n at different conjugation site of the cell-binding molecule can be different when the cytotoxic molecules containing the same or different bis-linkers are conjugated to a cell-binding molecule sequentially, or when different cytotoxic molecules containing the same or different bis-linkers are added step wisely in a conjugation reaction mixture containing a cell-binding molecule.

Formulation and Application

The conjugates of the patent application are formulated to liquid, or suitable to be lyophilized and subsequently be reconstituted to a liquid formulation. A liquid formulation comprising 0.1 g/L˜300 g/L of concentration of the conjugate active ingredient for delivery to a patient without high levels of antibody aggregation may include one or more polyols (e.g. sugars), a buffering agent with pH 4.5 to 7.5, a surfactant (e.g. polysorbate 20 or 80), an antioxidant (e.g. ascorbic acid and/or methionine), a tonicity agent (e.g. mannitol, sorbitol or NaCl), chelating agents such as EDTA; metal complexes (e.g. Zn-protein complexes); biodegradable polymers such as polyesters; a preservative (e.g. benzyl alcohol) and/or a free amino acid.

Suitable buffering agents for use in the formulations include, but are not limited to, organic acid salts such as salts of citric acid, ascorbic acid, gluconic acid, carbonic acid, tartaric acid, succinic acid, acetic acid or phthalic acid; Tris, tromethamine (tris(hydroxymethyl)-aminomethane) hydrochloride, or phosphate buffer. In addition, amino acid components can also be used as buffering agent. Such amino acid component includes without limitation arginine, glycine, glycylglycine, and histidine. The arginine buffers include arginine acetate, arginine chloride, arginine phosphate, arginine sulfate, arginine succinate, etc. In one embodiment, the arginine buffer is arginine acetate. Examples of histidine buffers include histidine chloride-arginine chloride, histidine acetate-arginine acetate, histidine phosphate-arginine phosphate, histidine sulfate-arginine sulfate, histidine succinate-argine succinate, etc. The formulations of the buffers have a pH of 4.5 to pH 7.5, preferably from about 4.5 to about 6.5, more preferably from about 5.0 to about 6.2. In some embodiments, the concentration of the organic acid salts in the buffer is from about 10 mM to about 500 mM.

A “polyol” that may optionally be included in the formulation is a substance with multiple hydroxyl groups. Polyols can be used as stabilizing excipients and/or isotonicity agents in both liquid and lyophilized formulations. Polyols can protect biopharmaceuticals from both physical and chemical degradation pathways. Preferentially excluded co-solvents increase the effective surface tension of solvent at the protein interface whereby the most energetically favorable structural conformations are those with the smallest surface areas. Polyols include sugars (reducing and nonreducing sugars), sugar alcohols and sugar acids. A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Sugar alcohols are selected from mannitol, xylitol, erythritol, maltitol, lactitol, erythritol, threitol, sorbitol and glycerol. Sugar acids include L-gluconate and its metallic salts thereof. Preferably, a nonreducing sugar: sucrose or trehalose at a concentration of about from 0.01% to 20% is chosen in the formulation, wherein trehalose being preferred over sucrose, because of the solution stability of trehalose.

A surfactant optionally in the formulations is selected from polysorbate (polysorbate 20, polysorbate 40, polysorbate 65, polysorbate 80, polysorbate 81, polysorbate 85 and the like); poloxamer (e.g. poloxamer 188, poly(ethylene oxide)-poly(propylene oxide), poloxamer 407 or polyethylene-polypropylene glycol and the like); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; and the MONAQUAT™ series (e.g. isostearyl ethylimidonium ethosulfate); polyethyl glycol, polypropyl glycol, and copolymers of ethylene and propylene glycol (e.g. Pluronics, PF68 etc.); etc. Preferred surfactants are polyoxyethylene sorbitan fatty acid esters e.g. polysorbate 20, 40, 60 or 80 (Tween 20, 40, 60 or 80). The concentration of a surfactant is range from 0.0001% to about 1.0%. In certain embodiments, the surfactant concentration is from about 0.01% to about 0.1%. In one embodiment, the surfactant concentration is about 0.02%.

A “preservative” optionally in the formulations is a compound that essentially reduces bacterial action therein. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The preservative is less than 5% in the formulation. Preferably 0.01% to 1%. In one embodiment, the preservative herein is benzyl alcohol.

Suitable free amino acids optionally for use in the formulation, but are not limited to, are arginine, lysine, histidine, ornithine, isoleucine, leucine, alanine, glycine glutamic acid or aspartic acid. The inclusion of a basic amino acid is preferred i.e. arginine, lysine and/or histidine. If a composition includes histidine then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g. a hydrochloride salt, such as arginine-HCl. The concentration of an amino acid is range from 0.0001% to about 15.0%. Preferably 0.01% to 5%.

The formulations can optionally comprise methionine or ascorbic acid as an antioxidant at a concentration of about from 0.01 mg/ml to 5 mg/ml; The formulations can optionally comprise chelating agent, e.g., EDTA, EGTA, etc., at a concentration of about from 0.01 mM to 2 mM.

The final formulation can be adjusted to the preferred pH with an adjust agent (e.g. an acid, such as HCl, H₂SO₄, acetic acid, H₃PO₄, citric acid, etc., or a base, such as NaOH, KOH, NH₃OH, ethanolamine, diethanolamine or triethanol amine, sodium phosphate, potassium phosphate, trisodium citrate, tromethamine, etc.) and the formulation should be controlled “isotonic” which is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example.

Other excipients which may be useful in either a liquid or lyophilized formulation of the patent application include, for example, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluoronic F-127, cellulose, cyclodextrin, dextran (10, 40 and/or 70 kD), polydextrose, maltodextrin, ficoll, gelatin, hydroxypropylmeth, sodium phosphate, potassium phosphate, ZnCl₂, zinc, zinc oxide, sodium citrate, trisodium citrate, tromethamine, copper, fibronectin, heparin, human serum albumin, protamine, glycerin, glycerol, EDTA, metacresol, benzyl alcohol, phenol, polyhydric alcohols, or polyalcohols, hydrogenated forms of carbohydrate having a carbonyl group reduced to a primary or secondary hydroxyl group.

Other contemplated excipients, which may be utilized in the aqueous pharmaceutical compositions of the patent application include, for example, flavoring agents, antimicrobial agents, sweeteners, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4^{th} edition, Rowe et al., Eds., American Pharmaceuticals Association (2003); and Remington: the Science and Practice of Pharmacy, 21^{th} edition, Gennaro, Ed., Lippincott Williams & Wilkins (2005).

In a further embodiment, the invention provides a method for preparing a formulation comprising the steps of: (a) lyophilizing the formulation comprising the conjugates, excipients, and a buffer system to a powder; and (b) reconstituting the lyophilized mixture of step (a) in a reconstitution medium such that the reconstituted formulation is stable. The formulation of step (a) may further comprise a stabilizer and one or more excipients selected from a group comprising bulking agent, salt, surfactant and preservative as hereinabove described. As reconstitution media several diluted organic acids or water, i.e. sterile water, bacteriostatic water for injection (BWFI) or may be used. The reconstitution medium may be selected from water, i.e. sterile water, bacteriostatic water for injection (BWFI) or the group consisting of acetic acid, propionic acid, succinic acid, sodium chloride, magnesium chloride, acidic solution of sodium chloride, acidic solution of magnesium chloride and acidic solution of arginine, in an amount from about 10 to about 250 mM.

A liquid pharmaceutical formulation of the conjugates of the patent application should exhibit a variety of pre-defined characteristics. One of the major concerns in liquid drug products is stability, as proteins/antibodies tend to form soluble and insoluble aggregates during manufacturing and storage. In addition, various chemical reactions can occur in solution (deamidation, oxidation, clipping, isomerization etc.) leading to an increase in degradation product levels and/or loss of bioactivity. Preferably, a conjugate in either liquid or loyphilizate formulation should exhibit a shelf life of more than 18 months at 0-25° C. More preferred a conjugate in either liquid or loyphilizate formulation should exhibit a shelf life of more than 24 months at 0-25° C. Most preferred liquid formulation should exhibit a shelf life of about 24 to 36 months at 2-8° C. and the loyphilizate formulation should exhibit a shelf life of about preferably up to 60 months at 2-8° C. Both liquid and loyphilizate formulations preferably exhibit a shelf life for at least two years at 0-8°, −20° C., or −70° C.

In certain embodiments, the formulation is stable following freezing (e. g., −20° C., or −70° C.) and thawing of the formulation, for example following 1, 2 or 3 cycles of freezing and thawing. Stability can be evaluated qualitatively and/or quantitatively in a variety of different ways, including evaluation of drug/antibody (protein) ratio and aggregate formation (for example using UV, size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis, or matrix-assisted laser desorption ionization/time-of-flight mass spectrometry (MALDI/TOF MS), or HPLC-MS/MS; CE-SDS or SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A stable conjugate should also “retains its biological activity” in a pharmaceutical formulation, if the biological activity of the conjugate at a given time, e. g. 12 month, within about 20%, preferably about 10% (within the errors of the assay) of the biological activity exhibited at the time the pharmaceutical formulation was prepared as determined in an antigen binding assay, and/or in vitro, cytotoxic assay, for example.

A pharmaceutical container or vessel is used to hold the pharmaceutical formulation of any of conjugates of the patent application. The vessel is a vial, bottle, pre-filled syringe, or pre-filled auto-injector syringe.

For clinical in vivo use, the conjugate via the bis-linkage of the invention will be supplied as solutions or as a lyophilized solid that can be redissolved in sterile water for injection. Examples of suitable protocols of conjugate administration are as follows. Conjugates are given daily, weekly, biweekly, triweekly, once every four weeks or monthly for 8-54 weeks as an i.v. bolus. Bolus doses are given in 50 to 1000 ml of normal saline to which human serum albumin (e.g. 0.5 to 1 mL of a concentrated solution of human serum albumin, 100 mg/mL) can optionally be added. Dosages will be about 50 μg/kg to 30 mg/kg of body weight per week, biweekly, or triweekly i.v. (range of 10 μg to 200 mg/kg per injection). 4-54 weeks after treatment, the patient may receive a second course of treatment. Specific clinical protocols with regard to route of administration, excipients, diluents, dosages, times, etc., can be determined by the skilled clinicians.

Examples of medical conditions that can be treated according to the in vivo or ex vivo methods of killing selected cell populations include malignancy of any types of cancer, autoimmune diseases, graft rejections, and infections (viral, bacterial or parasite).

The amount of a conjugate which is required to achieve the desired biological effect, will vary depending upon a number of factors, including the chemical characteristics, the potency, and the bioavailability of the conjugates, the type of disease, the species to which the patient belongs, the diseased state of the patient, the route of administration, all factors which dictate the required dose amounts, delivery and regimen to be administered.

In general terms, the conjugates via the bis-linkers of this invention may be provided in an aqueous physiological buffer solution containing 0.1 to 10% w/v conjugates for parenteral administration. Typical dose ranges are from 1 μg/kg to 0.1 g/kg of body weight daily; weekly, biweekly, triweekly, or monthly, a preferred dose range is from 0.01 mg/kg to 30 mg/kg of body weight weekly, biweekly, triweekly, or monthly, an equivalent dose in a human. The preferred dosage of drug to be administered is likely to depend on such variables as the type and extent of progression of the disease or disorder, the overall health status of the particular patient, the relative biological efficacy of the compound selected, the formulation of the compound, the route of administration (intravenous, intramuscular, or other), the pharmacokinetic properties of the conjugates by the chosen delivery route, and the speed (bolus or continuous infusion) and schedule of administrations (number of repetitions in a given period of time).

The conjugates via the linkers of the present invention are also capable of being administered in unit dose forms, wherein the term “unit dose” means a single dose which is capable of being administered to a patient, and which can be readily handled and packaged, remaining as a physically and chemically stable unit dose comprising either the active conjugate itself, or as a pharmaceutically acceptable composition, as described hereinafter. As such, typical total daily/weekly/biweekly/monthly dose ranges are from 0.01 to 100 mg/kg of body weight. By way of general guidance, unit doses for humans range from 1 mg to 3000 mg per day, or per week, per two weeks (biweekly), triweekly, or per month. Preferably the unit dose range is from 1 to 500 mg administered one to four times a month, and even more preferably from 1 mg to 100 mg, once a week, or once biweekly, or once triweekly. Conjugates provided herein can be formulated into pharmaceutical compositions by admixture with one or more pharmaceutically acceptable excipients. Such unit dose compositions may be prepared for use by oral administration, particularly in the form of tablets, simple capsules or soft gel capsules; or intranasal, particularly in the form of powders, nasal drops, or aerosols; or dermally, for example, topically in ointments, creams, lotions, gels or sprays, or via transdermal patches.

In yet another embodiment, a pharmaceutical composition comprising a therapeutically effective amount of the conjugate of Formula (I) or any conjugates described through the present patent can be administered concurrently with the other therapeutic agents such as the chemotherapeutic agent, the radiation therapy, immunotherapy agents, autoimmune disorder agents, anti-infectious agents or the other conjugates for synergistically effective treatment or prevention of a cancer, or an autoimmune disease, or an infectious disease. The synergistic agents are preferably selected from one or several of the following drugs: Abatacept, abemaciclib, Abiraterone acetate, Abraxane, Aducanumab, Acetaminophen/hydrocodone, Acalabrutinib, aducanumab, Adalimumab, ADXS31-142, ADXS-HER2, afatinib dimaleate, aldesleukin, alectinib, alemtuzumab, allitinib, Alitretinoin, ado-trastuzumab emtansine, Amphetamine/dextroamphetamine, anastrozole, apatinib, Aripiprazole, anthracyclines, Aripiprazole, Atazanavir, Atezolizumab, Atorvastatin, Avelumab, AVXS-101, Axicabtagene ciloleucel, axitinib, belinostat, BCG Live, Bevacizumab, bexarotene, blinatumomab, Bortezomib, bosutinib, brentuximab vedotin, brigatinib, Brolucizumab, Budesonide, Budesonide/formoterol, Buprenorphine, BYL719 (alpha-specific PI3K inhibitor), Cabazitaxel, Cabozantinib, capmatinib, Capecitabine, carfilzomib, chimeric antigen receptor-engineered T (CAR-T) cells, Celecoxib, ceritinib, Cetuximab, chiauranib, Chidamide, Ciclosporin, Cinacalcet, crizotinib, Cobimetinib, Cosentyx, crizotinib, Tisagenlecleucel, Dabigatran, dabrafenib, dacarbazine, daclizumab, dacomotinib, daptomycin, Daratumumab, Darbepoetin alfa, Darunavir, dasatinib, denileukin diftitox, Denosumab, Depakote, Dexlansoprazole, Dexmethylphenidate, Dexamethasone, DigniCap Cooling System, L-3,4-dihydroxyphenyl-alanine, Dinutuximab, dornase alfa, Doxycycline, Duloxetine, Duvelisib, durvalumab, elotuzumab, emicizumab, Emtricibine/Rilpivirine/Tenofovir, disoproxil fumarate, Emtricitbine/tenofovir/efavirenz, Enoxaparin, ensartinib, Enzalutamide, epitinib, Epoetin alfa, erlotinib, Esomeprazole, Eszopiclone, Etanercept, Everolimus, exemestane, everolimus, exenatide ER, Ezetimibe, Ezetimibe/simvastatin, famitinib, Fenofibrate, Filgotinib, Filgrastim, fingolimod, flumatinib, Fluticasone propionate, Fluticasone/salmeterol, fruquintinib, fulvestrant, gazyva, gefitinib, Glatiramer, Goserelin acetate, GSK2857916 (BCMA-ADC), henatinib, Icotinib, Imatinib, Ibritumomab tiuxetan, ibrutinib, icotinib, idelalisib, ifosfamide, Infliximab, imiquimod, ImmuCyst, Immuno BCG, iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, lapatinib, Ipilimumab, Ipratropium bromide/salbutamol, Ixazomib, Kanuma, Lanadelumab, Lanreotide acetate, lenalidomide, lenaliomide, lenvatinib mesylate, letrozole, Levothyroxine, Levothyroxine, Lidocaine, Linezolid, Liraglutide, Lisdexamfetamine, LN-144 (tumor-infiltrating lymphocyte), lorlatinib, lucitanib/delitinib, Memantine, Methoxy polyethylene glycol-epoetin beta, Methylphenidate, Metoprolol, Mekinist, mericitabine/Rilpivirine/Tenofovir, Modafinil, Mometasone, Mycidac-C, mycophenolic acid, Necitumumab, neratinib, Nilotinib, niraparib, Nivolumab, ofatumumab, obinutuzumab, ocrelizumab, olaparib, Olmesartan, Olmesartan/hydrochlorothiazide, Omalizumab, Omega-3 fatty acid ethyl esters, Oncorine, Oseltamivir, Osimertinib, Oxycodone, Ozanimod, palbociclib, Palivizumab, panitumumab, panobinostat, pazopanib, pembrolizumab, PD-1 antibody, PD-L1 antibody, Pemetrexed, pertuzumab, Pirfenidone, Pneumococcal conjugate vaccine, pomalidomide, Pregabalin, ProscaVax, Propranolol, puquitinib, pyrotinib, Quetiapine, Rabeprazole, radium 223 chloride, Raloxifene, Raltegravir, ramucirumab, Ranibizumab, regorafenib, ribociclib, Risankizumab, Rituximab, Rivaroxaban, romidepsin, Rosuvastatin, ruxolitinib phosphate, Salbutamol, savolitinib, semaglutide, Sevelamer, Sildenafil, siltuximab, simotinib, sipatinib/cipatinib, Siponimod, Sipuleucel-T, Sitagliptin, Sitagliptin/metformin, Solifenacin, solanezumab, Sonidegib, Sorafenib, sulfatinib, Sunitinib, tacrolimus, tacrimus, Tadalafil, tamoxifen, Tafinlar, Talimogene laherparepvec, talazoparib, Telaprevir, talazoparib, Temozolomide, temsirolimus, Tenecteplase, Tenofovir/emtricitabine, tenofovir disoproxil fumarate, Testosterone gel, tezacaftor/ivacaftor, Thalidomide, theliatinib, TICE BCG, Tiotropium bromide, Tisagenlecleucel, Tocilizumab, toremifene, trametinib, Trastuzumab, Trabectedin (ecteinascidin 743), trametinib, tremelimumab, Trifluridine/tipiracil, Tretinoin, Upadacitinib, Uro-BCG, Ustekinumab, Valoctocogene roxaparvovec, Valsartan, veliparib, vandetanib, vemurafenib, venetoclax, vismodegib, volitinib, vorinostat, ziv-aflibercept, Zostavax, and their analogs, derivatives, pharmaceutically acceptable salts, carriers, diluents, or excipients thereof, or a combination above thereof.

The drugs/cytotoxic agents used for conjugation via a bridge linker of the present patent can be any analogues and/or derivatives of drugs/molecules described in the present patent. One skilled in the art of drugs/cytotoxic agents will readily understand that each of the drugs/cytotoxic agents described herein can be modified in such a manner that the resulting compound still retains the specificity and/or activity of the starting compound. The skilled artisan will also understand that many of these compounds can be used in place of the drugs/cytotoxic agents described herein. Thus, the drugs/cytotoxic agents of the present invention include analogues and derivatives of the compounds described herein. All references cited herein and in the examples that follow are expressly incorporated by reference in their entireties.

### EXAMPLES

The invention is further described in the following examples, which are not intended to limit the scope of the invention. Cell lines described in the following examples were maintained in culture according to the conditions specified by the American Type Culture Collection (ATCC) or Deutsche Sammlung von Mikroorganismen und Zellkulturen GmbH, Braunschweig, Germany (DMSZ), or The Shanghai Cell Culture Institute of Chinese Academy of Science, unless otherwise specified. Cell culture reagents were obtained from Invitrogen Corp., unless otherwise specified. All anhydrous solvents were commercially obtained and stored in Sure-seal bottles under nitrogen. All other reagents and solvents were purchased as the highest grade available and used without further purification. The preparative HPLC separations were performed with Varain PreStar HPLC. NMR spectra were recorded on Varian Mercury 400 MHz Instrument. Chemical shifts (.delta.) are reported in parts per million (ppm) referenced to tetramethylsilane at 0.00 and coupling constants (J) are reported in Hz. The mass spectral data were acquired on a Waters Xevo QTOF mass spectrum equipped with Waters Acquity UPLC separations module and Acquity TUV detector.

### Example 1. Synthesis of methyl 2,5-dioxo-2,5-dihydro-1H-pyrrole-1-carboxylate

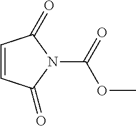

To a solution of maleimide (6.35 g, 65.4 mmol, 1.0 eq.) in EtOAc (120 mL) were added N-methyl morpholine (8.6 mL, 78.5 mmol, 1.2 eq.) and methyl chloroformate (6.0 mL, 78.5 mmol, 1.2 eq.) at 0° C. The reaction was stirred at 0° C. for 30 min and r.t. 1 h. The solid was filtered off and filtrate concentrated. The residue was dissolved in CH₂Cl₂ and filtered through a silica gel plug and eluted with CH₂Cl₂ to remove the color. The appropriate fractions were concentrated and resulted solid was triturated with 10% EtOAc/PE to give a white solid of the title compound (9.00 g, 89% yield).

### Example 2. Synthesis of (S)-3-((tert-butoxycarbonyl)amino)-2-(2,5-dioxo-2,5-dihydro-1H-pyrrol-1-yl)propanoic Acid

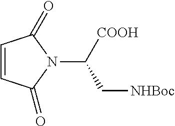 [SMILES: CC(C)(C)OC(=O)NC[C@@H](C(=O)O)N1C(=O)C=CC1=O]

To a solution of H-Dap(Boc)-OH (1.00 g, 4.9 mmol) in saturated NaHCO₃ (20 mL) at 0° C. was added methyl 2,5-dioxo-2,5-dihydro-1H-pyrrole-1-carboxylate (2.30 g, 14.7 mmol). The reaction was stirred at 0° C. for 1h, then warmed to r.t. and stirred for another hour. Then 1N KHSO₄ was added to adjust pH to ˜6 and the resulting mixture was extracted with EtOAc (2×50 mL). Combined organic layers were dried over Na₂SO₄, filtered, and concentrated to give the title compound (0.42 g, 30% yield). ESI m/z calcd for C₁₂H₁₅N₂O₆ [M−H]⁻; 283.10, found 283.10.

### Example 3. Synthesis of tert-butyl (2-(2,5-dioxo-2,5-dihydro-1H-pyrrol-1-yl)ethyl)carbamate

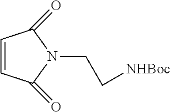 [SMILES: CC(C)(C)OC(=O)NCCN1C(=O)C=CC1=O]

A mixture of N-Boc-ethylenediamine (5.6 mL, 35.4 mmol, 1.1 eq.) and saturated NaHCO₃ (60 mL) was cooled to 0° C., to which methyl 2,5-dioxo-2,5-dihydro-1H-pyrrole-1-carboxylate (5.00 g, 32.2 mmol, 1.0 eq.) was added in portions. After stirring at 0° C. for 30 min, the reaction was warmed to r.t. and stirred for 1 h. The precipitate was collected by filtration and washed with cold water, then dissolved in EtOAc and washed with brine, dried over anhydrous Na₂SO₄ and concentrated to give the title compound as white solid (6.69 g, 87% yield).

### Example 4. Synthesis of tert-butyl (2-(1,3-dioxo-3a,4,7,7a-tetrahydro-1H-4,7-epoxyisoindol-2(3H)-yl)ethyl)carbamate

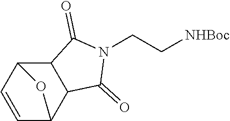 [SMILES: CC(C)(C)OC(=O)NCCN1C(=O)C2C3C=CC(O3)C2C1=O]

A solution of tert-butyl (2-(2,5-dioxo-2,5-dihydro-1H-pyrrol-1-yl)ethyl)carbamate (6.00 g, 25.0 mmol), furan (18.0 mL) in toluene (120 mL) in a high pressure tube was heated to reflux and stirred for 16 h. The colorless solution turned yellow during reaction. The mixture was then cooled to r.t. and concentrated. The resulting white solid was triturated with ethyl ether to give the title compound (6.5 g, 84% yield).

### Example 5. Synthesis of 2-(2-aminoethyl)-3a,4,7,7a-tetrahydro-1H-4,7-epoxyisoindole-1,3(2H)-dione hydrochloride

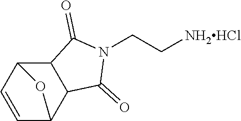

A solution of tert-butyl 2-(1,3-dioxo-3a,4,7,7a-tetrahydro-1H-4,7-epoxyisoindol-2(3H)-yl)ethyl)carbamate. (9.93 g, 32.2 mmol) was dissolved in dioxane (15 mL) and treated with concentrated HCl (15 mL) at r.t. for 3 h. The reaction was concentrated and the resulting solid was collected by filtration, with washing of the filter cake with EtOAc. The solid was dried in an oven (50° C.) overnight to give the title compound (6.94 g, 88% yield).

### Example 6. Synthesis of tert-butyl 2,8-dioxo-1,5-oxazocane-5-carboxylate

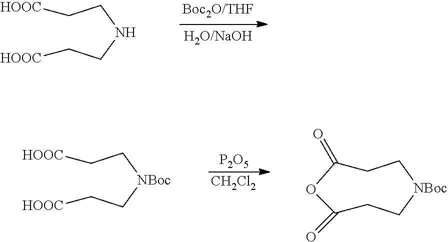

To a solution of 3,3′-azanediyl dipropanoic acid (10.00 g, 62.08 mmol) in 1.0 M NaOH (300 ml) at 4° C. was added di-tert-butyl dicarbonate (22.10 g, 101.3 mmol) in 200 ml THF in 1 h. After addition, the mixture was kept to stirring for 2 h at 4° C. The mixture was carefully acidified to pH ˜4 with 0.2 M H₃PO₄, concentrated in vacuo, extracted with CH₂Cl₂, dried over Na₂SO₄, evaporated and purified with flash SiO₂ chromatography eluted with AcOH/MeOH/CH₂Cl₂ (0.01:1:5) to afford 3,3′-((tert-butoxycarbonyl)azanediyl)dipropanoic acid (13.62 g, 84% yield). ESI MS m/z C₁₁H₁₉NO₆ [M+H]⁺, cacld. 262.27, found 262.40.

To a solution of 3,3′-((tert-butoxycarbonyl)azanediyl)dipropanoic acid (8.0 g, 30.6 mmol) in CH₂Cl₂ (500 ml) at 0° C. was added phosphorus pentoxide (8.70 g, 61.30 mmol). The mixture was stirred at 0° C. for 2 h and then r.t. for 1 h, filtered through short SiO₂ column, and rinsed the column with EtOAc/CH₂Cl₂ (1:6). The filtrate was concentrated and triturated with EtOAc/hexane to afford the title compound (5.64 g, 74% yield). ESI MS m/z C₁₁H₁₇NO₅ [M+H]⁺, cacld. 244.11, found 244.30.

### Example 7. Synthesis of tert-Butyl 3-(2-(2-(2-(tosyloxy)ethoxy)ethoxy)ethoxy)propanoate

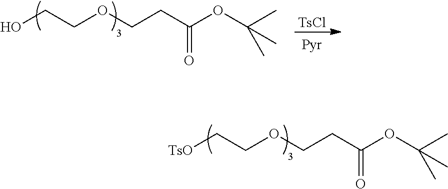

A solution of tert-Butyl 3-(2-(2-(2-hydroxyethoxy)ethoxy)ethoxy)propanoate (10.0 g, 35.95 mmol) in acetonitrile (50.0 mL) was treated with pyridine (20.0 mL). A solution of tosyl chloride (7.12 g, 37.3 mmol) in 50 mL acetonitrile was added dropwise via an addition funnel over 30 minutes. After 5 h TLC analysis revealed that the reaction was complete. The pyridine hydrochloride that had formed was filtered off and the solvent was removed. The residue was purified on silica gel by eluting from with 20% ethyl acetate in hexane to with neat ethyl acetate to give 11.2 g (76% yield) of the title compound. ¹H NMR: 1.40 (s, 9H), 2.40 (s, 3H), 2.45 (t, 2H, J=6.4 Hz), 3.52-3.68 (m, 14H), 4.11 (t, 2H, J=4.8 Hz), 7.30 (d, 2H, J=8.0 Hz), 7.75 (d, 2H, J=8.0 Hz); ESI MS m/z+C₂₀H₃₃O₈S (M+H), cacld. 433.18, found 433.30.

### Example 8. Synthesis of tert-Butyl 3-(2-(2-(2-azidoethoxy)ethoxy)ethoxy)propanoate

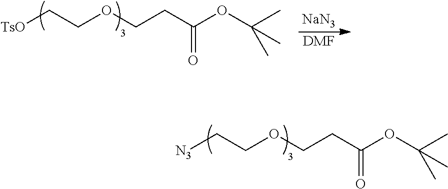

To 50 mL of DMF was added tert-butyl 3-(2-(2-(2-(tosyloxy)ethoxy)ethoxy)ethoxy)-propanoate (4.0 g, 9.25 mmol) and sodium azide (0.737 g, 11.3 mmol) with stirring. The reaction was heated to 80° C. After 4 h TLC analysis revealed that the reaction was complete. The reaction was cooled to room temperature and quenched with water (25 mL). The aqueous layer was separated and extracted into ethyl acetate (3×35 mL). The combined organic layers were dried over anhydrous magnesium sulfate, filtered, and the solvent removed in vacuo. The crude azide product (2.24 g, 98% yield, about 93% pure by HPLC) was used for next step without further purification. ¹H NMR (CDCl₃): 1.40 (s, 9H), 2.45 (t, 2H, J=6.4 Hz), 3.33 (t, 2H, J=5.2 Hz), 3.53-3.66 (m, 12H). ESI MS m/z+C₁₃H₂₆N₃O₈ (M+H), cacld. 304.18, found 304.20.

### Example 9. Synthesis of 3-(2-(2-(2-azidoethoxy)ethoxy)ethoxy)propanoic Acid

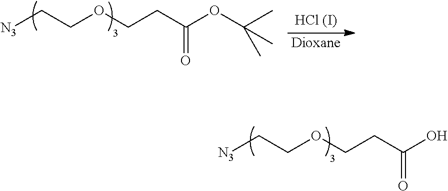

Tert-butyl 3-(2-(2-(2-azidoethoxy)ethoxy)ethoxy)propanoate (2.20 g, 7.25 mmol) in 1,4-dioxane (40 ml) was added HCl (12 M, 10 ml). The mixture was stirred for 40 min, diluted with dioxane (20 ml) and toluene (40 ml), evaporated and co-evaporated with dioxane (20 ml) and toluene (40 ml) to dryness to afford the crude title product for the next step without further production (1.88 g, 105% yield, ˜92% pure by HPLC). MS ESI m/z calcd for C₉H₁₈N₃O₅ [M+H]⁺ 248.12, found 248.40.

### Example 10. Synthesis of 13-amino-4,7,10-trioxadodecanoic Acid Tert-Butyl Ester, and 13-Amino-bis(4,7,10-trioxadodecanoic Acid Tert-Butyl Ester)

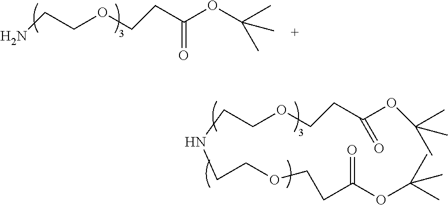

The crude azide material 3-(2-(2-(2-azidoethoxy)ethoxy)ethoxy)propanoic acid (5.0 g, ˜14.84 mmol) was dissolved in ethanol (80 mL) and 300 mg of 10% Pd/C was added. The system was evacuated under vacuum and placed under 2 atm of hydrogen gas via hydrogenation reactor with vigorous stirring. The reaction was then stirred overnight at room temperature and TLC showed that the starting materials disappeared. The crude reaction was passed through a short pad of Celite rinsing with ethanol. The solvent was removed and the amine purified on silica gel using a mixture of methanol (from 5% to 15%) and 1% triethylamine in methylene chloride as the eluant to give 13-amino-4,7,10-trioxadodecanoic acid tert-butyl ester (1.83 g, 44% yield, ESI MS m/z+C₁₃H₂₇NO₅ (M+H), cacld. 278.19, found 278.30) and 13-amino-bis(4,7,10-trioxadodecanoic acid tert-butyl ester) (2.58 g, 32% yield, ESI MS m/z+C₂₆H₅₂NO₁₀ (M+H), cacld. 538.35, found 538.40).

### Example 11. Synthesis of 3-(2-(2-(2-Aminoethoxy)ethoxy)ethoxy)propanoic Acid, HCl Salt

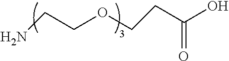

To 13-amino-4,7,10-trioxadodecanoic acid tert-butyl ester (0.80 g, 2.89 mmol) in 30 mL of dioxane was 10 ml of HCl (36%) with stirring. After 0.5 h TLC analysis revealed that the reaction was complete, the reaction mixture was evaporated, and co-evaporated with EtOH and EtOH/Toluene to form the title product in HCl salt (>90% pure, 0.640 g, 86% yield) without further purification. ESI MS m/z+C₉H₂₀NO₅ (M+H), cacld. 222.12, found 222.20.

### Example 12. 13-Amino-bis(4,7,10-trioxadodecanoic Acid, HCl Salt

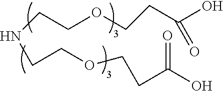

To 13-amino-bis(4,7,10-trioxadodecanoic acid tert-butyl ester) (1.00 g, 1.85 mmol) in 30 mL of dioxane was 10 ml of HCl (36%) with stirring. After 0.5 h TLC analysis revealed that the reaction was complete, the reaction mixture was evaporated, and co-evaporated with EtOH and EtOH/Toluene to form the title product in HCl salt (>90% pure, 0.71 g, 91% yield) without further purification. ESI MS m/z+C₁₈H₃₆NO₁₀ (M+H), cacld. 426.22, found 426.20.

### Example 13. Synthesis of tert-butyl 3-(2-(2-(2-hydroxyethoxy)ethoxy)ethoxy) propanoate

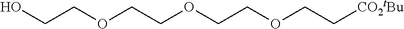 [SMILES: CC(C)(C)OC(=O)CCOCCOCCOCCO]

To a solution of 2,2′-(ethane-1,2-diylbis(oxy))diethanol (55.0 mL, 410.75 mmol, 3.0 eq.) in anhydrous THE (200 mL) was added sodium (0.1 g). The mixture was stirred until Na disappeared and then tert-butyl acrylate (20.0 mL, 137.79 mmol, 1.0 eq.) was added dropwise. The mixture was stirred overnight and then quenched by HCl solution (20.0 mL, 1N) at 0° C. THE was removed by rotary evaporation, brine (300 mL) was added and the resulting mixture was extracted with EtOAc (3×100 mL). The organic layers were washed with brine (3×300 mL), dried over anhydrous Na₂SO₄, filtered and concentrated to afford a colourless oil (30.20 g, 79.0% yield), which was used without further purification. MS ESI m/z calcd for C₁₃H₂₇O₆ [M+H]⁺ 278.1729, found 278.1730.

### Example 14. Synthesis of tert-butyl 3-(2-(2-(2-(tosyloxy)ethoxy)ethoxy)ethoxy) propanoate

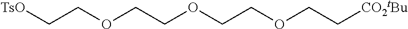 [SMILES: Cc1ccc(S(=O)(=O)OCCOCCOCCOCCC(=O)OC(C)(C)C)cc1]

To a solution of tert-butyl 3-(2-(2-(2-hydroxyethoxy)ethoxy)ethoxy) propanoate (30.20 g, 108.5 mmol, 1.0 eq.) and TsCl (41.37 g, 217.0 mmol, 2.0 eq.) in anhydrous DCM (220 mL) at 0° C. was added TEA (30.0 mL, 217.0 mmol, 2.0 eq.). The mixture was stirred at room temperature overnight, and then washed with water (3×300 mL) and brine (300 mL), dried over anhydrous Na₂SO₄, filtered, concentrated and purified by SiO₂ column chromatography (3:1 hexanes/EtOAc) to give a colourless oil (39.4 g, 84.0% yield). MS ESI m/z calcd for C₂₀H₃₃O₈S [M+H]⁺ 433.1818, found 433.2838.

### Example 15. Synthesis of tert-butyl 3-(2-(2-(2-azidoethoxy)ethoxy)ethoxy) propanoate

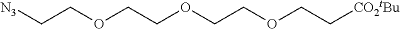 [SMILES: CC(C)(C)OC(=O)CCOCCOCCOCCN=[N+]=[N-]]

To a solution of tert-butyl 3-(2-(2-(2-(tosyloxy)ethoxy)ethoxy)ethoxy) propanoate (39.4 g, 91.1 mmol, 1.0 eq.) in anhydrous DMF (100 mL) was added NaN₃ (20.67 g, 316.6 mmol, 3.5 eq.). The mixture was stirred at room temperature overnight. Water (500 mL) was added and extracted with EtOAc (3×300 mL). The combined organic layers were washed with water (3×900 mL) and brine (900 mL), dried over anhydrous Na₂SO₄, filtered, concentrated and purified by SiO₂ column chromatography (5:1 hexanes/EtOAc) to give a light yellow oil (23.8 g, 85.53% yield). MS ESI m/z calcd for C₁₃H₂₅O₃N₅Na [M+Na]⁺ 326.2, found 326.2.

### Example 16. Synthesis of tert-butyl 3-(2-(2-(2-aminoethoxy)ethoxy)ethoxy) propanoate

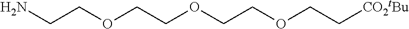 [SMILES: CC(C)(C)OC(=O)CCOCCOCCOCCN]

Raney-Ni (7.5 g, suspended in water) was washed with water (three times) and isopropyl alcohol (three times) and mixed with tert-butyl 3-(2-(2-(2-azidoethoxy)ethoxy)ethoxy) propanoate (5.0 g, 16.5 mmol) in isopropyl alcohol. The mixture was stirred under a H₂ balloon at r.t. for 16 h and then filtered over a Celite pad, with washing of the pad with isopropyl alcohol. The filtrate was concentrated and purified by column chromatography (5-25% MeOH/DCM) to give a light yellow oil (2.60 g, 57% yield). MS ESI m/z calcd for C₁₃H₂₈NO₅ [M+H]⁺ 279.19; found 279.19.

### Example 17. Synthesis of 2-(2-(dibenzylamino)ethoxy)ethanol

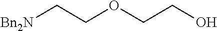

2-(2-aminoethoxy)ethanol (21.00 g, 200 mmol, 1.0 eq.) and K₂CO₃(83.00 g, 600 mmol, 3.0 eq.) in acetonitrile (350 mL) was added BnBr (57.0 mL, 480 mmol, 2.4 eq.). The mixture was refluxed overnight. Water (1 L) was added and extracted with EtOAc (3×300 mL). The combined organic layers were washed with brine (1000 mL), dried over anhydrous Na₂SO₄, filtered, concentrated and purified by SiO₂ column chromatography (4:1 hexanes/EtOAc) to give a colourless oil (50.97 g, 89.2% yield). MS ESI m/z calcd for C₁₈H₂₃NO₂Na [M+Na]⁺ 309.1729, found 309.1967.

### Example 18. Synthesis of tert-butyl 3-(2-(2-(dibenzylamino)ethoxy)ethoxy) propanoate

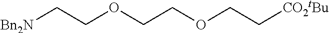

To a mixture of 2-(2-(dibenzylamino)ethoxy)ethanol (47.17 g, 165.3 mmol, 1.0 eq.), tert-butyl acrylate (72.0 mL, 495.9 mmol, 3.0 eq.) and n-Bu₄NI (6.10 g, 16.53 mmol, 0.1 eq.) in DCM (560 mL) was added sodium hydroxide solution (300 mL, 50%). The mixture was stirred overnight. The organic layer was separated and the water layer was extracted with EtOAc (3×100 mL). The organic layers were washed with water (3×300 mL) and brine (300 mL), dried over anhydrous Na₂SO₄, filtered, concentrated and purified by SiO₂ column chromatography (7:1 hexanes/EtOAc) to give a colourless oil (61.08 g, 89.4% yield). MS ESI m/z calcd for C₂₅H₃₆NO₄ [M+H]⁺ 414.2566, found 414.2384.

### Example 19. Synthesis of tert-butyl 3-(2-(2-aminoethoxy)ethoxy)propanoate

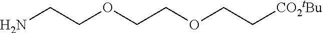 [SMILES: CC(C)(C)OC(=O)CCOCCOCCN]

To a solution of tert-butyl 3-(2-(2-(dibenzylamino)ethoxy)ethoxy) propanoate (20.00 g, 48.36 mmol, 1.0 eq.) in THE (30 mL) and MeOH (60 mL) was added Pd/C (2.00 g, 10 wt %, 50% wet) in a hydrogenation bottle. The mixture was shaken at 1 atom pressure H₂ overnight, filtered through Celite (filter aid), and the filtrate was concentrated to afford a colourless oil (10.58 g, 93.8% yield). MS ESI m/z calcd for C₁₁H₂₄NO₄ [M+H]⁺ 234.1627, found 234.1810.

### Example 20. Synthesis of tert-butyl 3-(2-(2-hydroxyethoxy)ethoxy)propanoate

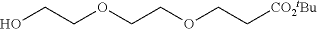 [SMILES: CC(C)(C)OC(=O)CCOCCOCCO]

To a solution of 2,2′-oxydiethanol (19.7 mL, 206.7 mmol, 3.0 eq.) in anhydrous THE (100 mL) was added sodium (0.1 g). The mixture was stirred until Na disappeared and then tert-butyl acrylate (10.0 mL, 68.9 mmol, 1.0 eq.) was added dropwise. The mixture was stirred overnight, and brine (200 mL) was added and extracted with EtOAc (3×100 mL). The organic layers were washed with brine (3×300 mL), dried over anhydrous Na₂SO₄, filtered, concentrated and purified by SiO₂ column chromatography (1:1 hexanes/EtOAc) to give to a colourless oil (8.10 g, 49.4% yield). MS ESI m/z calcd for C₁₁H₂₃O₅[M+H]⁺ 235.1467, found 235.1667.

### Example 21. Synthesis of tert-butyl 3-(2-(2-(tosyloxy)ethoxy)ethoxy)propanoate

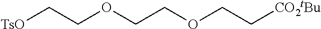 [SMILES: Cc1ccc(S(=O)(=O)OCCOCCOCCC(=O)OC(C)(C)C)cc1]

To a solution of tert-butyl 3-(2-(2-hydroxyethoxy)ethoxy)propanoate (6.24 g, 26.63 mmol, 1.0 eq.) and TsCl (10.15 g, 53.27 mmol, 2.0 eq.) in anhydrous DCM (50 mL) at 0° C. was added pyridine (4.3 mL, 53.27 mmol, 2.0 eq.). The mixture was stirred at room temperature overnight, and then washed with water (100 mL) and the water layer was extracted with DCM (3×50 mL). The combined organic layers were washed with brine (300 mL), dried over anhydrous Na₂SO₄, filtered, concentrated and purified by SiO₂ column chromatography (5:1 hexanes/EtOAc) to give a colourless oil (6.33 g, 61.3% yield). MS ESI m/z calcd for C₁₈H₂₇O₇S [M+H]⁺ 389.1556, found 389.2809.

### Example 22. Synthesis of tert-butyl 3-(2-(2-azidoethoxy)ethoxy)propanoate

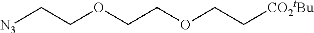 [SMILES: CC(C)(C)OC(=O)CCOCCOCCN=[N+]=[N-]]

To a solution of tert-butyl 3-(2-(2-(tosyloxy)ethoxy)ethoxy)propanoate (5.80 g, 14.93 mmol, 1.0 eq.) in anhydrous DMF (20 mL) was added NaN₃ (5.02 g, 77.22 mmol, 5.0 eq.). The mixture was stirred at room temperature overnight. Water (120 mL) was added and extracted with EtOAc (3×50 mL). The combined organic layers were washed with water (3×150 mL) and brine (150 mL), dried over anhydrous Na₂SO₄, filtered, concentrated and purified by SiO₂ column chromatography (5:1 hexanes/EtOAc) to give a colourless oil (3.73 g, 69.6% yield). MS ESI m/z calcd for C₁₁H₂₂O₃N₄Na [M+H]⁺ 260.1532, found 260.2259.

### Example 23. Synthesis of tert-butyl 3-(2-(2-aminoethoxy)ethoxy)propanoate

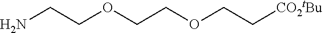 [SMILES: CC(C)(C)OC(=O)CCOCCOCCN]

tert-Butyl 3-(2-(2-azidoethoxy)ethoxy)propanoate (0.18 g, 0.69 mmol) was dissolved in MeOH (3.0 mL, with 60 μL concentrated HCl) and hydrogenated with Pd/C (10 wt %, 20 mg) under a H₂ balloon for 30 min. The catalyst was filtered through a Celite pad, with washing of the pad with MeOH. The filtrate was concentrated to give a colorless oil (0.15 g, 93% yield). MS ESI m/z calcd for C₁₁H₂₄NO₄ [M+H]⁺ 234.16; found 234.14.

### Example 24. Synthesis of 3-(2-(2-azidoethoxy)ethoxy)propanoic Acid

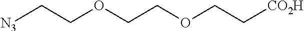 [SMILES: [N-]=[N+]=NCCOCCOCCC(=O)O]

tert-Butyl 3-(2-(2-azidoethoxy)ethoxy)propanoate (2.51 g, 9.68 mmol) dissolved in 1,4-dioxane (30 mL) was treated with 10 ml of HCl (conc.) at r.t. The mixture was stirred for 35 min, diluted with EtOH (30 ml) and toluene (30 ml) and concentrated under vacuum. The crude mixture was purified on silica gel using a mixture of methanol (from 5% to 10%) and 1% formic acid in methylene chloride as the eluant to give title compound (1.63 g, 83% yield), ESI MS m/z C₇H₁₂N₃O₄ [M−H], cacld. 202.06, found 202.30.

### Example 25. Synthesis of 2,5-dioxopyrrolidin-1-yl 3-(2-(2-azidoethoxy)ethoxy)propanoate

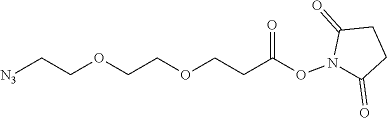 [SMILES: [N-]=[N+]=NCCOCCOCCC(=O)ON1C(=O)CCC1=O]

To 3-(2-(2-azidoethoxy)ethoxy)propanoic acid (1.60 g, 7.87 mmol) in 30 mL of dichloromethane was added NHS (1.08 g, 9.39 mmol) and EDC (3.60 g, 18.75 mmol) with stirring. After 8 h TLC analysis revealed that the reaction was complete, the reaction mixture was concentrated and purified on silica gel using a mixture of ethyl acetate (from 5% to 10%) in methylene chloride as the eluant to give title compound (1.93 g, 82% yield). ESI MS m/z C₁₁H₁₇N₄O₆ [M+H]⁺, cacld. 301.11, found 301.20.

### Example 26. Synthesis of 2,5-dioxopyrrolidin-1-yl 3-(2-(2-(2-azidoethoxy)ethoxy)ethoxy) propanoate

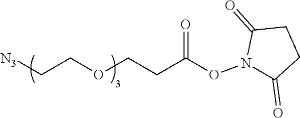

To 3-(2-(2-(2-azidoethoxy)ethoxy)ethoxy)propanoic acid (4.50 g, 18.21 mmol) in 80 mL of dichloromethane was added NHS (3.0 g, 26.08 mmol) and EDC (7.60 g, 39.58 mmol) with stirring. After 8 h TLC analysis revealed that the reaction was complete, the reaction mixture was concentrated and purified on silica gel using a mixture of ethyl acetate (from 5% to 10%) in methylene chloride as the eluant to give title compound (5.38 g, 86% yield). ESI MS m/z C₁₃H₂₀N₄O₇ [M+H]⁺, cacld. 345.13, found 345.30.

### Example 27. Synthesis of (14S,17S)-1-azido-17-(2-(tert-butoxy)-2-oxoethyl)-14-(4-((tert-butoxycarbonyl)-amino)butyl)-12,15-dioxo-3,6,9-trioxa-13,16-diazaoctadecan-18-oic Acid

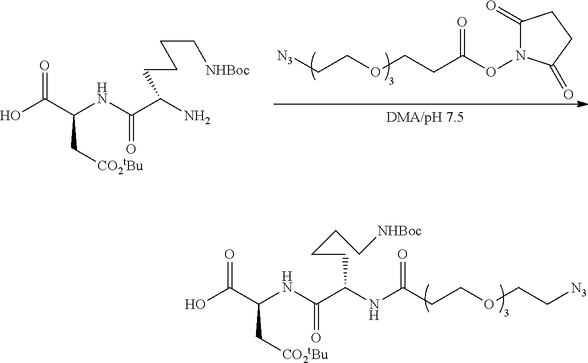

To a solution of (S)-2-((S)-2-amino-6-((tert-butoxycarbonyl)amino)hexanamido)-4-(tert-butoxy)-4-oxobutanoic acid (2.81 g, 6.73 mmol) in the mixture of DMA (70 ml) and 0.1 M NaH₂PO₄ (50 ml, pH 7.5) was added 2,5-dioxopyrrolidin-1-yl 3-(2-(2-(2-azidoethoxy)ethoxy)-ethoxy)propanoate (3.50 g, 10.17). The mixture was stirred for 4 h, evaporated in vacuo, purified on silica gel using a mixture of methanol (from 5% to 15%) in methylene chloride containing 0.5% acetic acid as the eluant to give title compound (3.35 g, 77% yield). ESI MS m/z C₂₈H₅₁N₆O₁₁ [M+H]⁺, cacld. 647.35, found 647.80.

### Example 28. Synthesis of (14S,17S)-tert-butyl 1-azido-14-(4-((tert-butoxycarbonyl)-amino)butyl)-17-((4-(hydroxymethyl)phenyl)carbamoyl)-12,15-dioxo-3,6,9-trioxa-13,16-diazanonadecan-19-oate

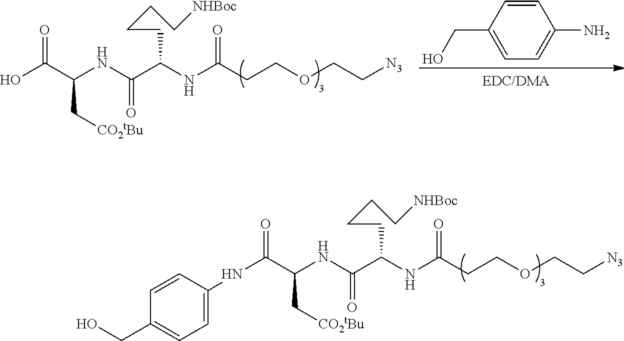

(14S,17S)-1-azido-17-(2-(tert-butoxy)-2-oxoethyl)-14-(4-((tert-butoxycarbonyl)-amino)butyl)-12,15-dioxo-3,6,9-trioxa-13,16-diazaoctadecan-18-oic acid (3.30 g, 5.10 mmol) and (4-aminophenyl)methanol (0.75 g, 6.09) in DMA (25 ml) was added EDC (2.30 g, 11.97 mmol). The mixture was stirred for overnight, evaporated in vacuo, purified on silica gel using a mixture of methanol (from 5% to 8%) in methylene chloride containing as the eluant to give title compound (3.18 g, 83% yield). ESI MS m/z C₃₅H₅₈N₇O₁₁ [M+H]⁺, cacld. 752.41, found 752.85.

### Example 29. Synthesis of (14S,17S)-tert-butyl 1-amino-14-(4-((tert-butoxycarbonyl)-amino)butyl)-17-((4-(hydroxymethyl)phenyl)carbamoyl)-12,15-dioxo-3,6,9-trioxa-13,16-diazanonadecan-19-oate

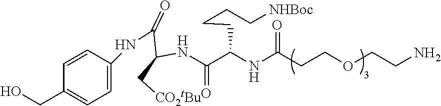

To a solution of (14S,17S)-tert-butyl 1-azido-14-(4-((tert-butoxycarbonyl)amino)butyl)-17-((4-(hydroxymethyl)phenyl)carbamoyl)-12,15-dioxo-3,6,9-trioxa-13,16-diazanonadecan-19-oate (1.50 g, 1.99 mmol) in THE (35 mL) was added Pd/C (200 mg, 10% Pd, 50% wet) in a hydrogenation bottle. The mixture was shaken at 1 atom pressure H₂ overnight, filtered through Celite (filter aid), and the filtrate was concentrated to afford the title compound (1.43 g, 99% yield) which was used immediately for the next step without further purification. ESI MS m/z C₃₅H₆₀N₅O₁₁ [M+H]⁺, cacld. 726.42, found 726.70.

### Example 30. Synthesis of (S)-15-azido-5-isopropyl-4,7-dioxo-10,13-dioxa-3,6-diazapentadecan-1-oic Acid

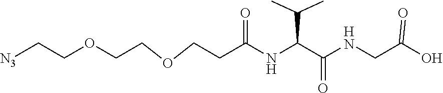 [SMILES: CC(C)[C@H](NC(=O)CCOCCOCCN=[N+]=[N-])C(=O)NCC(=O)O]

To a solution of (S)-2-(2-amino-3-methylbutanamido)acetic acid (Val-Gly) (1.01 g, 5.80 mmol) in the mixture of DMA (50 ml) and 0.1 M NaH₂PO₄ (50 ml, pH 7.5) was added 2,5-dioxopyrrolidin-1-yl 3-(2-(2-azidoethoxy)ethoxy)propanoate (1.90 g, 6.33). The mixture was stirred for 4 h, evaporated in vacuo, purified on silica gel using a mixture of methanol (from 5% to 15%) in methylene chloride containing 0.5% acetic acid as the eluant to give title compound (1.52 g, 73% yield). ESI MS m/z C₁₄H₂₆N₅O₆ [M+H]⁺, cacld. 360.18, found 360.40.

### Example 31. Synthesis of (S)-2,5-dioxopyrrolidin-1-yl 15-azido-5-isopropyl-4,7-dioxo-10,13-dioxa-3,6-diazapentadecan-1-oate

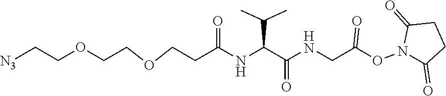 [SMILES: CC(C)[C@H](NC(=O)CCOCCOCCN=[N+]=[N-])C(=O)NCC(=O)ON1C(=O)CCC1=O]

To a solution of (S)-15-azido-5-isopropyl-4,7-dioxo-10,13-dioxa-3,6-diazapentadecan-1-oic acid (1.50 g, 4.17 mmol) in 40 mL of dichloromethane was added NHS (0.88 g, 7.65 mmol) and EDC (2.60 g, 13.54 mmol) with stirring. After 8 h TLC analysis revealed that the reaction was complete, the reaction mixture was concentrated and purified on silica gel using a mixture of ethyl acetate (from 5% to 20%) in methylene chloride as the eluant to give title compound (1.48 g, 78% yield). ESI MS m/z C₁₈H₂₉N₆O₈ [M+H]⁺, cacld. 457.20, found 457.50.

### Example 32. Synthesis of 4-(((benzyloxy)carbonyl)amino)butanoic Acid

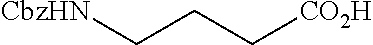 [SMILES: O=C(O)CCCNC(=O)OCc1ccccc1]

A solution of 4-aminobutyric acid (7.5 g, 75 mmol) and NaOH (6 g, 150 mmol) in H₂O (40 mL) was cooled to 0° C. and treated with a solution of CbzCl (16.1 g, 95 mmol) in THE (32 ml) dropwise. After 1 h, the reaction was allowed to warm to r.t. and stirred for 3 h. THE was removed under vacuum, the pH of the aqueous solution was adjusted to 1.5 by addition of 6 N HCl. Extracted with ethyl acetate, and the organic layer was washed with brine, dried and concentrated to give the title compound (16.4 g, 92% yield). MS ESI m/z calcd for C₁₂H₁₆NO₅ [M+H]⁺ 238.10, found 238.08.

### Example 33. Synthesis of tert-butyl 4-(((benzyloxy)carbonyl)amino)butanoate

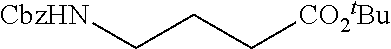 [SMILES: CC(C)(C)OC(=O)CCCNC(=O)OCc1ccccc1]

DMAP (0.8 g, 6.56 mmol) and DCC (17.1 g, 83 mmol) were added to a solution of 4-(((benzyloxy)carbonyl)amino)butanoic acid (16.4 g, 69.2 mmol) and t-BuOH (15.4 g, 208 mmol) in DCM (100 mL). After stirring at r.t. overnight, the reaction was filtered and filtrate concentrated. The residue was dissolved in ethyl acetate and the washed with 1N HCl, brine and dried over Na₂SO₄. Concentration and purification by column chromatography (10 to 50% EtOAc/hexanes) yielded the title compound (7.5 g, 37% yield). MS ESI m/z calcd for C₁₆H₂₃NO₄Na [M+Na]⁺ 316.16, found 316.13.

### Example 34. Synthesis of tert-butyl 4-aminobutanoate

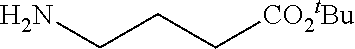 [SMILES: CC(C)(C)OC(=O)CCCN]

tert-Butyl 4-(((benzyloxy)carbonyl)amino)butanoate (560 mg, 1.91 mmol) was dissolved in MeOH (50 mL), and mixed with Pd/C catalyst (10 wt %, 100 mg) then hydrogenated (1 atm) at room temperature for 3 h. The catalyst was filtered off and all volatiles were removed under vacuum to afford the title compound (272 mg, 90% yield). MS ESI m/z calcd for C₈H₁₈NO₂ [M+H]⁺ 160.13, found 160.13.

### Example 35. Synthesis of di-tert-butyl 3,3′-(benzylazanediyl)dipropanoate

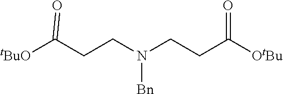 [SMILES: CC(C)(C)OC(=O)CCN(CCC(=O)OC(C)(C)C)Cc1ccccc1]

A mixture of phenylmethanamine (2.0 mL, 18.29 mmol, 1.0 eq) and tert-butyl acrylate (13.3 mL, 91.46 mmol, 5.0 eq) was refluxed at 80° C. overnight and then concentrated. The crude product was purified by SiO₂ column chromatography (20:1 hexanes/EtOAc) to give the title compound as colourless oil (5.10 g, 77% yield). ESI MS m/z: calcd for C₂₁H₃₄NO₄ [M+H]⁺ 364.2, found 364.2. ¹H NMR (400 MHz, CDCl₃) δ 7.38-7.21 (m, 5H), 3.58 (s, 2H), 2.76 (t, J=7.0 Hz, 4H), 2.38 (t, J=7.0 Hz, 4H), 1.43 (s, 17H).

### Example 36. Synthesis of di-tert-butyl 3,3′-azanediyldipropanoate

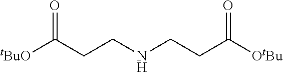 [SMILES: CC(C)(C)OC(=O)CCNCCC(=O)OC(C)(C)C]

To a solution of di-tert-butyl 3,3′-(benzylazanediyl)dipropanoate (1.37 g, 3.77 mmol, 1.0 equiv) in MeOH (10 mL) was added Pd/C (0.20 g, 10% Pd/C, 50% wet) in a hydrogenation bottle. The mixture was shaken overnight under H₂ atmosphere and then filtered through a Celite pad. The filtrate was concentrated to give the title compound as colourless oil (1.22 g, 89% yield). ESI MS m/z: calcd for C₁₄H₂₈NO₄ [M+H]⁺ 274.19, found 274.20.

### Example 37. Synthesis of tert-butyl 4-(2-(((benzyloxy)carbonyl)amino)propan amido)-butanoate

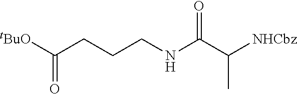 [SMILES: CC(NC(=O)OCc1ccccc1)C(=O)NCCCC(=O)OC(C)(C)C]

To a solution of tert-butyl 4-aminobutanoate (1.00 g, 6.28 mmol, 1.0 eq.) and Z-L-alaine (2.10 g, 9.42 mmol, 1.5 eq.) in anhydrous DCM (50 mL) at 0° C. were added HATU (3.10 g, 8.164 mmol, 1.3 eq.) and TEA (2.6 mL, 18.8 mmol, 3.0 eq.). The reaction was stirred at 0° C. for 10 min., then warmed to room temperature and stirred overnight. The mixture was diluted with DCM and washed with water and brine, dried over anhydrous Na₂SO₄, concentrated and purified by SiO₂ column chromatography (10:3 petroleum ether/ethyl acetate) to give the title compound as a colorless oil (1.39 g, 61% yield). ESI MS m/z: calcd for C₁₉H₂₉N₂O₅Na [M+H]⁺ 387.2, found 387.2.

### Example 38. Synthesis of tert-butyl 4-(2-aminopropanamido)butanoate

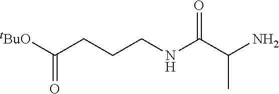 [SMILES: CC(N)C(=O)NCCCC(=O)OC(C)(C)C]

To a solution of tert-butyl 4-(2-(((benzyloxy)carbonyl)amino)propanamido) butanoate (1.39 g, 3.808 mmol, 1.0 eq.) in MeOH (12 mL) was added Pd/C (0.20 g, 10 wt %, 10% wet) in a hydrogenation bottle. The mixture was shaken for 2 h and then filtered through Celite (filter aid), concentrated to give the title compound as a light yellow oil (0.838 g, 95% yield). ESI MS m/z: calcd. for C₁₁H₂₃N₂O₃ [M+H]⁺ 231.16, found 231.15.

### Example 39. Synthesis of 3-(2(2-(dibenzylamino)ethoxy)ethoxy)propanoic Acid

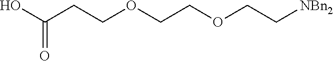

To a solution of tert-butyl 3-(2-(2-(dibenzylamino)ethoxy)ethoxy)propanoate (2.3 g, 5.59 mmol, 1.0 eq) in DCM (10 mL) at room temperature was added TFA (5 mL). After stirring for 90 min., the reaction mixture was diluted with anhydrous toluene and concentrated, this operation was repeated for three times to give the title compound as a light yellow oil (2.0 g, theoretical yield), which was directly used in the next step. ESI MS m/z calcd. for C₂₁H₂₈NO₄ [M+H]⁺ 358.19, found 358.19.

### Example 40. Synthesis of perfluorophenyl 3-(2-(2-(dibenzylamino)ethoxy) ethoxy)-propanoate

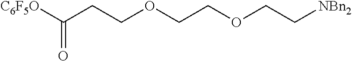

To a solution of 3-(2-(2-(dibenzylamino)ethoxy)ethoxy)propanoic acid (2.00 g, 5.59 mmol, 1.0 eq.) in anhydrous DCM (30 mL) at 0° C. was added DIPEA until pH was neutral, and then PFP (1.54 g, 8.38 mmol, 1.5 eq.) and DIC (1.04 mL, 6.70 mmol, 1.2 eq.) were added. After 10 min. the reaction was warmed to room temperature and stirred overnight. The mixture was filtered, concentrated and purified by SiO₂ column chromatography (15:1 petroleum ether/ethyl acetate) to give the title compound as colourless oil (2.10 g, 72% yield). ESI MS m/z: calcd. for C₂₇H₂₇F₅NO₄ [M+H]⁺ 524.2, found 524.2.

### Example 41. Synthesis of tert-butyl 2-benzyl-13-methyl-11,14-dioxo-1-phenyl-5,8-dioxa-2,12,15-triazanonadecan-19-oate

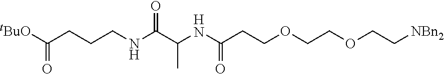

To a solution of tert-butyl 4-(2-aminopropanamido)butanoate (0.736 g, 3.2 mmol, 1.0 eq.) and perfluorophenyl 3-(2-(2-(dibenzylamino)ethoxy) ethoxy)propanoate (2.01 g, 3.84 mmol, 1.2 eq.) in anhydrous DMA (20 mL) at 0° C. was added DIPEA (1.7 mL, 9.6 mmol, 3.0 eq.). After stirring at 0° C. for 10 min. the reaction was warmed to room temperature and stirred overnight. Water (100 mL) was added and the mixture was extracted with EtOAc (3×100 mL). The combined organic layers were washed with water (3×200 mL) and brine (200 mL), dried over Na₂SO₄, filtered, concentrated and purified by SiO₂ column chromatography (25:2 DCM/MeOH) to give the title compound as a colourless oil (1.46 g, 80% yield). ESI MS m/z: calcd. for C₃₂H₄₈N₃O₆ [M+H]⁺ 570.34, found 570.33.

### Example 42. Synthesis of 2-benzyl-13-methyl-11,14-dioxo-1-phenyl-5,8-dioxa-2,12,15-triazanonadecan-19-oic Acid

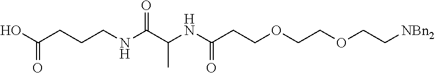

To a solution of tert-butyl 2-benzyl-13-methyl-11,14-dioxo-1-phenyl-5,8-dioxa-2,12,15-triazanonadecan-19-oate (0.057 g, 0.101 mmol, 1.0 eq) in DCM (3 mL) at room temperature was added TFA (1 mL) and stirred for 40 min. The reaction was diluted with anhydrous toluene and then concentrated. This operation was repeated three times to give the title compound as a colourless oil (0.052 g, theoretical yield), which was used directly in the next step. ESI MS m/z: calcd for C₂₈H₄₀N₃O₆ [M+H]⁺ 514.28, found 514.28.

### Example 43. Synthesis of tert-butyl 2-(2-(((benzyloxy)carbonyl)amino)propanamido)-acetate

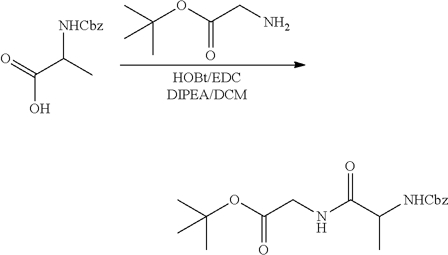

2-(((Benzyloxy)carbonyl)amino)propanoic acid (0.84 g, 5 mmol), tert-butyl 2-aminoacetate (0.66 g, 5 mmol), HOBt (0.68 g, 5 mmol), EDC (1.44 g, 7.5 mmol) were dissolved in DCM (20 ml), followed by addition of DIPEA (1.7 ml, 10 mmol). The reaction mixture was stirred at RT overnight, washed with H₂O (100 ml), and the aqueous layer was extracted with EtOAc. The organic layers were combined, dried over MgSO₄, filtered, evaporated under reduced pressure and the residue was purified on SiO₂ column to give the title product 1(0.87 g, 52%). ESI: m/z: calcd for C₁₇H₂₅N₂O₅[M+H]⁺: 337.17, found 337.17.

### Example 44. Synthesis of 2-(2-(((benzyloxy)carbonyl)amino)propanamido)acetic Acid

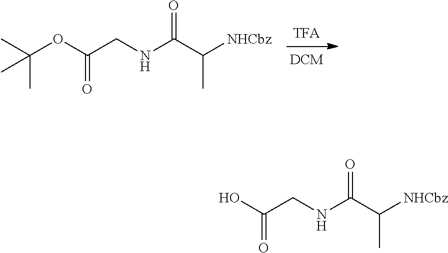 [SMILES: CC(NC(=O)OCc1ccccc1)C(=O)NCC(=O)OC(C)(C)C ; CC(NC(=O)OCc1ccccc1)C(=O)NCC(=O)O]

Tert-butyl 2-(2-(((benzyloxy)carbonyl)amino)propanamido)acetate (0.25 g, 0.74 mmol) was dissolved in DCM (30 ml), followed by addition of TFA (10 ml). The mixture was stirred at RT overnight, concentrated to afford the title compound used for the next step without further purification. ESI: m/z: calcd for C₁₃H₁₇N₂O₅ [M+H]⁺: 281.11, found 281.60.

### Example 45. Synthesis of tert-Butyl 3-(2-(2-(2-hydroxyethoxy)ethoxy)ethoxy)propanoate

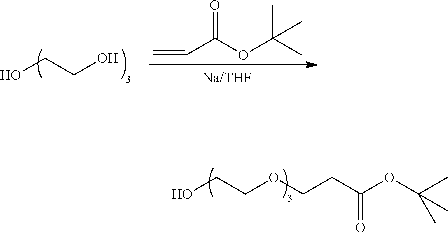

To 350 mL of anhydrous THF was added 80 mg (0.0025 mol) of sodium metal and triethylene glycol 150.1 g, 1.00 mol) with stirring. After the sodium had completely dissolved, tert-butyl acrylate (24 mL, 0.33 mol) was added. The solution was stirred for 20 h at room temperature and neutralized with 8 mL of 1.0 M HCl. The solvent was removed in vacuo and the residue was suspended in brine (250 mL) and extracted with ethyl acetate (3×125 mL). The combined organic layers were washed with brine (100 mL) then water (100 mL), dried over sodium sulfate, and the solvent was removed. The resulting colorless oil was dried under vacuum to give 69.78 g (76% yields) of the title product. ¹H NMR: 1.41 (s, 9H), 2.49 (t, 2H, J=6.4 Hz), 3.59-3.72 (m, 14H). ESI MS m/z—C₁₃H₂₅O₆(M−H), cacld. 277.17, found 277.20.

### Example 46. Synthesis of tert-butyl 2,5,8,11,14,17,20,23,26,29-decaoxahentriacontan-31-oate

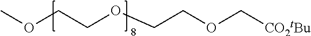

NaH (60%, 8.0 g, 200 mmol) was added to a solution of 2,5,8,11,14,17,20,23,26-nonaoxaoctacosan-28-ol (42.8 g, 100 mmol) in THE (1.0 L). After stirring at r.t. for 30 min, tert-butyl 2-bromoacetate (48.8 g, 250 mmol) was added to the mixture, and stirred at r.t. for 1 h. The mixture was then poured onto ice water, extracted with DCM, and the organic layer was washed with brine, dried over anhydrous Na₂SO₄. Purification by column chromatography (0% to 5% MeOH: DCM) yielded compound 432 as a yellow oil (32 g, 59% yield).

### Example 47. Synthesis of 2,5,8,11,14,17,20,23,26,29-decaoxahentriacontan-31-oic Acid

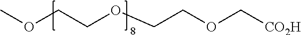

Tert-butyl 2,5,8,11,14,17,20,23,26,29-decaoxahentriacontan-31-oate (40.0 g, 73.8 mmol) was dissolved in DCM (400 mL), and then formic acid (600 mL) was added. The resulting solution was stirred at 25° C. overnight. All volatiles were removed under vacuum, which afforded the title product as a yellow oil (36.0 g, theoretical yield). ESI m/z calcd for C₂₁H₄₃O₁₂ [M+H]⁺: 487.27, found 487.24.

### Example 48. Synthesis of 2,5,8,11,14,17,20,23,26,29-decaoxahentriacontan-31-oyl chloride

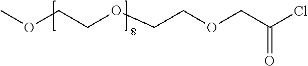

To the solution of 2,5,8,11,14,17,20,23,26,29-decaoxahentriacontan-31-oic acid (36.0 g, 73.8 mmol) dissolved in DCM (640 mL), (COCl)₂ (100 mL) and DMF (52 g, 0.74 mmol) were added. The resulting solution was stirred at r.t. for 4 h. All volatiles were removed under vacuum to yield the title product as a yellow oil.

### Example 49. Synthesis of (S)-37-(((benzyloxy)carbonyl)amino)-31-oxo-2,5,8,11,14,17,20,23,26,29-decaoxa-32-azaoctatriacontan-38-oic acid

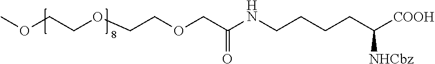

Z-L-Lys-OH (41.4 g, 147.6 mmol, Na₂CO₃ (2.4 g, 221.4 mmol) and NaOH (5.9 g, 147.6 mmol) were dissolved in water (720 mL). The mixture was cooled to 0° C., to which a solution of 2,5,8,11,14,17,20,23,26,29-decaoxahentriacontan-31-oyl chloride (37.2 g, 73.8 mmol) in THE (20 mL) was added. The resulting mixture was stirred at r.t. for 1 h. THF was removed under vacuum, and concentrated HCl was added to the aqueous solution until pH reached 3 under ice cooling. After extraction with DCM, the organic layer was washed with brine, dried over Na₂SO₄ and concentrated to give the title product as a yellow oil (55 g, 99% yield). ESI m/z calcd for C₃₅H₆₀N₂O₁₅ [M+H]⁺: 749.40, found 749.39.

### Example 50. Synthesis of (S)-tert-butyl 13-(2-(((benzyloxy)carbonyl)amino)-5-(tert-butoxy)-5-oxopentanamido)tridecanoate

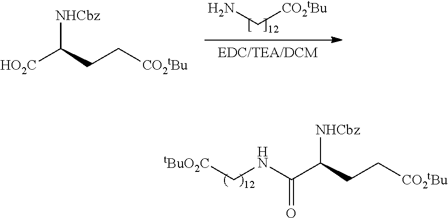

To the solution of (S)-2-(((benzyloxy)carbonyl)amino)-5-(tert-butoxy)-5-oxopentanoic acid (3.50 g, 10.38 mmol) and tert-butyl 13-aminotridecanoate (3.00 g, 10.51 mmol) in DMF (70 mL) was added EDC (10.00 g, 52.08 mmol) and TEA (1.60 mL, 11.16 mmol). The reaction was stirred at room temperature for 8 h, concentrated in vacuo, diluted with NaCl saturated water (80 ml) and EtOAc (100 ml), separated. The aqeuous layer was extracted with EtOAc (50 mL×3) and the combined organic phases were washed once with 100 mL of saturated brine, then dried over anhydrous Na₂SO₄, filtered and concentrated. The residue was purified by SiO₂ column chromatography (EtOAc/DCM, 1:15) to afford the title compound (5.45 g, 87% yield), ESI: m/z: calcd for C₃₄H₅₇N₂O₇ [M+H]⁺: 605.41, found 605.38.

### Example 51. Synthesis of (S)-tert-butyl 13-(2-amino-5-(tert-butoxy)-5-oxopentanamido)tridecanoate

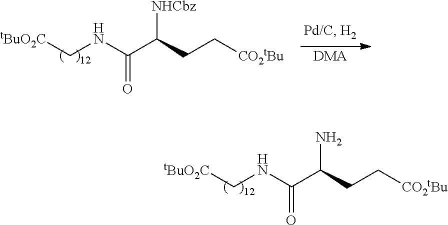

To a solution of S)-tert-butyl 13-(2-(((benzyloxy)carbonyl)amino)-5-(tert-butoxy)-5-oxopentanamido)tridecanoate (2.80 g, 4.63 mmol) in DMA (100 mL) was added 10% Pd/C (0.41 g), the mixture was stirred under hydrogen atmosphere at room temperature for 18 h. Then the Pd/C was removed by filtration through celite and the filter bed was washed with DMA. The filtrate was concentrated to afford the product as yellow foam which was used in the next step without further purification (2.19 g, 101% yield). ESI: m/z: calcd for C₂₆H₅₁N₂O₅[M+H]⁺: 471.37, found 471.80.

### Example 52. Synthesis of 2,2-dimethyl-4,17-dioxo-3,7,10,13,20,23,26-heptaoxa-16-azanonacosan-29-oic Acid

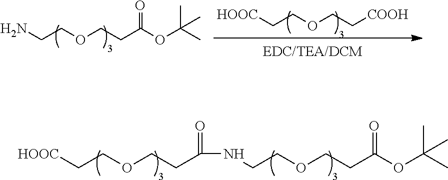

In a solution of tert-butyl 3-(2-(2-(2-aminoethoxy)ethoxy)ethoxy)propanoate (6.00 g, 21.64 mmol) and 3,3′-((oxybis(ethane-2,1-diyl))bis(oxy))dipropanoic acid (21.01 g, 84.00 mmol) in DMA (200 ml) were added EDC (18.00 g, 93.75 mmol) and DIPEA (5.00 g, 38.75 mmol). The mixture was stirred overnight, then concentrated and purified by SiO₂ column chromatography (MeOH:CH₂Cl₂=1:12 to 1:5) to give the title compound as a white oil (9.15 g, 86% yield). ESI m/z: calcd for C₂₃H₄₄NO₁₁ [M+H]⁺: 510.28, found: 510.55.

### Example 53. Synthesis of 1-benzyl 39-tert-butyl 14,26-dioxo-4,7,10,17,20,23,30,33,36-nonaoxa-13,27-diazanonatriacontane-1,39-dioate

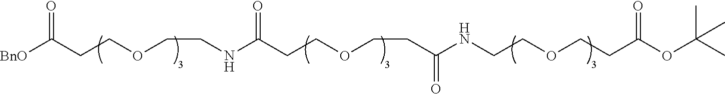

In a solution of (S)-tert-butyl 13-(2-amino-5-(tert-butoxy)-5-oxopentanamido)tridecanoate (5.11 g, 10.03 mmol) and benzyl 3-(2-(2-(2-aminoethoxy)ethoxy)ethoxy)propanoate (3.21 g, 10.31 mmol) in DMA (100 ml) were added EDC (8.02 g, 41.77 mmol) and DIPEA (3.00 g, 23.25 mmol). The mixture was stirred overnight, then concentrated and purified by SiO₂ column chromatography (EtOAc:CH₂Cl₂=1:8 to 1:3) to give the title compound as a white oil (7.01 g, 87% yield). ESI m/z: calcd for C₃₉H₆₇N₂O₁₅ [M+H]⁺: 803.44, found: 803.80.

### Example 54. Synthesis of3,16,28-trioxo-1-phenyl-2,6,9,12,19,22,25,32,35,38-decaoxa-15,29-diazahentetracontan-41-oic Acid

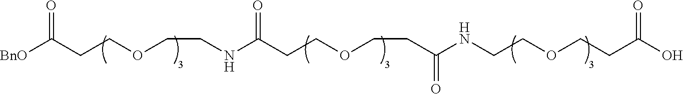

1-benzyl 39-tert-butyl 14,26-dioxo-4,7,10,17,20,23,30,33,36-nonaoxa-13,27-diazanonatriacontane-1,39-dioate (6.90 g, 8.60 mmol) was dissolved in HCOOH (50 mL) and stirred at 0-4° C. for 1 hour. The reaction mixture was diluted with toluene (50 ml), concentrated and co-evaporated with toluene twice, and the residue was placed on a vacuum pump to the title compound (6.45 g, ˜101% yield, crude product). ESI: m/z: calcd for C₃₅H₅₉N₂O₁₅ [M+H]⁺: 747.38, found 747.50.

### Example 55. Synthesis of 1-benzyl 39-(2,5-dioxopyrrolidin-1-yl) 14,26-dioxo-4,7,10,17,20,23,30,33,36-nonaoxa-13,27-diazanonatriacontane-1,39-dioate

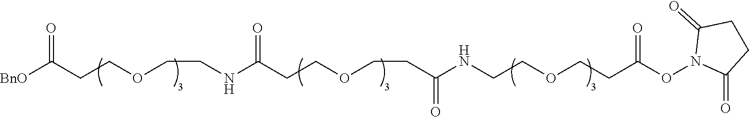

In a solution of 3,16,28-trioxo-1-phenyl-2,6,9,12,19,22,25,32,35,38-decaoxa-15,29-diazahentetracontan-41-oic acid (4.01 g, 5.37 mmol) and NHS (N-hydroxysuccinimde) (0.68 g, 5.91 mmol) in DMA (100 ml) were added EDC (1.52 g, 7.92 mmol) and DIPEA (0.50 g, 3.87 mmol). The mixture was stirred overnight, then concentrated and purified by SiO₂ column chromatography (EtOAc:CH₂Cl₂=1:8 to 1:4) to give the title compound as a white foam (4.17 g, 92% yield). ESI m/z: calcd for C₃₉H₆₂N₃O₁₇ [M+H]⁺: 844.40, found: 844.85.

### Example 56. Synthesis of (S)-47-(((benzyloxy)carbonyl)amino)-3,16,28,41-tetraoxo-1-phenyl-2,6,9,12,19,22,25,32,35,38-decaoxa-15,29,42-triazaoctatetracontan-48-oic Acid

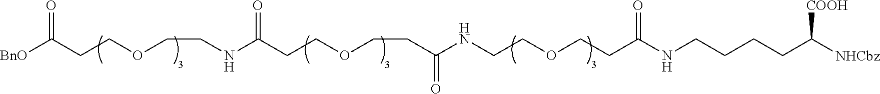

In a solution of (S)-6-amino-2-(((benzyloxy)carbonyl)amino)hexanoic acid (1.38 g, 4.92 mmol) in DMA (30 ml) and 100 mM NaH2PO4, pH 7.5 buffer (40 ml) was added 1-benzyl 39-(2,5-dioxopyrrolidin-1-yl) 14,26-dioxo-4,7,10,17,20,23,30,33,36-nonaoxa-13,27-diazanonatriacontane-1,39-dioate (4.15 g, 4.92 mmol) in 4 portions in 2 h. The mixture was stirred for 4 h, then concentrated and purified by SiO₂ column chromatography (MeOH:CH₂Cl₂=1:7 to 1:4) to give the title compound as a white foam (4.07 g, 82% yield). ESI m/z: calcd for C₄₉H₇₇N₄O₁₈ [M+H]⁺: 1009.51, found: 1009.90.

### Example 57. Synthesis of (S)-1-benzyl 51-(2-(trimethylsilyl)ethyl) 45-(((benzyloxy)-carbonyl)amino)-14,26,39,46-tetraoxo-4,7,10,17,20,23,30,33,36-nonaoxa-13,27,40,47-teraazahenpentacontane-1,51-dioate

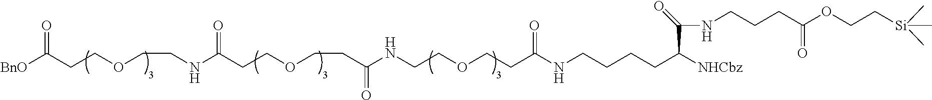

In a solution of (S)-47-(((benzyloxy)carbonyl)amino)-3,16,28,41-tetraoxo-1-phenyl-2,6,9,12,19,22,25,32,35,38-decaoxa-15,29,42-triazaoctatetracontan-48-oic acid (4.00 g, 3.96 mmol) and 2-(trimethylsilyl)ethyl 4-aminobutanoate (0.90 g, 4.43 mmol) in DMA (25 ml) was added EDC (2.03 g, 10.57 mmol). The mixture was stirred for 6 h, then concentrated and purified by SiO₂ column chromatography (MeOH:CH₂Cl₂=1:15 to 1:8) to give the title compound as a white foam (3.97 g, 84% yield). ESI m/z: calcd for C₅₈H₉₆N₅O₁₉Si [M+H]⁺: 1194.64, found: 1194.90.

### Example 58. Synthesis of 12-amino-2,2-dimethyl-6,11,18,31,43-pentaoxo-5,21,24,27,34,37,40,47,50,53-decaoxa-10,17,30,44-tetraaza-2-silahexapentacontan-56-oic Acid

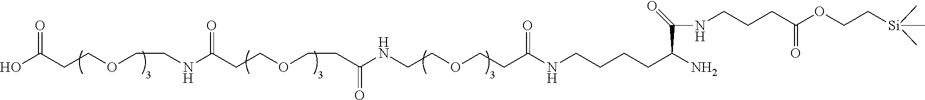

To a solution of (S)-1-benzyl 51-(2-(trimethylsilyl)ethyl) 45-(((benzyloxy)-carbonyl)amino)-14,26,39,46-tetraoxo-4,7,10,17,20,23,30,33,36-nonaoxa-13,27,40,47-tetraazahenpentacontane-1,51-dioate (3.90 g, 3.33 mmol) in MeOH (40 mL) was added Pd/C (10 wt %, 0.20 g) in a hydrogenation bottle. The mixture was shaken at 40 psi of H₂ for 2 h, filtered through Celite (filter aid), and the filtrate was concentrated to afford the title compound (3.16 g, 98% yield) which was used directly for the next step without further purification. ESI: m/z: calcd for C₄₃H₈₃N₅O₁₇Si [M+H]⁺: 970.55, found 970.70.

### Example 59. Synthesis of 2,5-dioxopyrrolidin-1-yl 4-((3aR,7R,7aS)-1,3-dioxo-3a,4,7,7a-tetrahydro-1H-4,7-epoxyisoindol-2(3H)-yl)butanoate

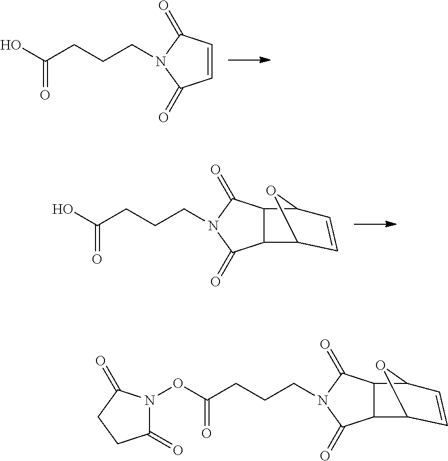 [SMILES: O=C(O)CCCN1C(=O)C=CC1=O ; O=C(O)CCCN1C(=O)C2C3C=CC(O3)C2C1=O ; O=C(CCCN1C(=O)C2C3C=CC(O3)C2C1=O)ON1C(=O)CCC1=O]

4-(2,5-dioxo-2,5-dihydro-1H-pyrrol-1-yl)butanoic acid (10.0 g, 54.62 mmol) and furan (5 ml, 68.74 mmol) in ether (90 ml) in a pressure vessel was heated at 170° C. for 6 h. Then the solution was cooled down to room temperature, concentrated in vacuo and crystalized in EtOH/Hexane to afford 4-((3aR,7R,7aS)-1,3-dioxo-3a,4,7,7a-tetrahydro-1H-4,7-epoxyisoindol-2(3H)-yl)butanoic acid (11.24 g, 44.76 mmol, 82% yield). Then the yield acid compound redisolved in CH₂Cl₂ (100 ml) was added NHS (7.00 g, 60.86 mmol) and EDC (25.00 g, 130.20 mmol). The mixture was stirred for 6 h, then concentrated and purified by SiO₂ column chromatography (EtOAc:CH₂Cl₂=1:8 to 1:5) to give the title compound as a white foam (13.57 g, 87% yield). ESI m/z: calcd for C₁₆H₁₇N₂O₇ [M+H]⁺: 349.09, found: 349.55.

### Example 60. Synthesis of 2,3-bis(2-bromoacetamido)succinyl dichloride

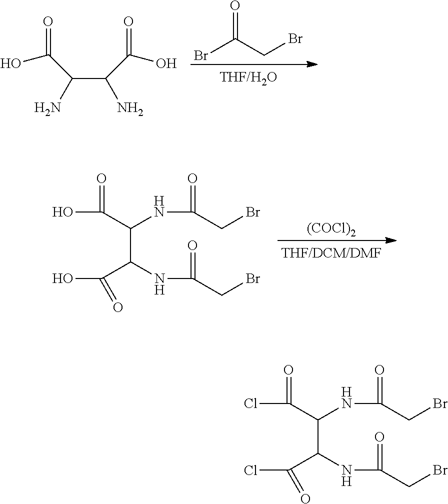

2,3-Diaminosuccinic acid (5.00 g, 33.77 mmol) in the mixture of THF/H₂O/DIPEA (125 ml/125 ml/8 ml) was added 2-bromoacetyl bromide (25.0 g, 125.09 mmol). The mixture was stirred for overnight, evaporated and purified by SiO₂ column chromatography (H₂O/CH₃CN 5:95) to afforded 2,3-bis(2-bromoacetamido)succinic acid (9.95 g, 76% yield) as light yellow oil. MS ESI m/z calcd for C₈H₁₁Br₂N₂O₆ [M+H]⁺ 388.89, found 388.68.

2,3-bis(2-bromoacetamido)succinic acid (3.50 g, 9.02 mmol) in dichloromethane (80 ml) was added oxalyl dichloride (5.80 g, 46.05 mmol) and DMF (0.01 ml). The mixture was stirred for 2.5 h, diluted with toluene, concentrated and co-evaporated with dichloroethane (2×20 ml) and toluene (2×15 ml) to dryness to afford 2,3-bis(2-bromoacetamido)succinyl dichloride as crude product (which is not stable) for the next step without further purification (3.90 g, 102% yield). MS ESI m/z calcd for C₈H₉Br₂Cl₂N₂O₄ [M+H]⁺ 424.82, found 424.90.

### Example 61. Synthesis of 2,3-bis(((benzyloxy)carbonyl)amino)succinic Acid

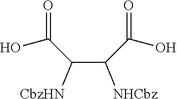 [SMILES: O=C(NC(C(=O)O)C(NC(=O)OCc1ccccc1)C(=O)O)OCc1ccccc1]

To a solution of 2,3-diaminosuccinic acid (4.05 g, 27.35 mmol) in the mixture of THF (250 ml) and NaH₂PO₄ (0.1 M, 250 ml, pH 8.0) was added benzyl carbonochloridate (15.0 g, 88.23 mmol) in 4 portions in 2 h. The mixture was stirred for another 6 h, concentrated and purified on SiO₂ column eluted with H₂O/CH₃CN (1:9) containing 1% formic acid to afford the title compound (8.65 g, 76% yield, ˜95% pure). MS ESI m/z calcd for C₂₀H₂₁N₂O₈ [M+H]⁺ 417.12, found 417.60.

### Example 62. Synthesis of bis(2,5-dioxopyrrolidin-1-yl) 2,3-bis(((benzyloxy)carbonyl)-amino)succinate

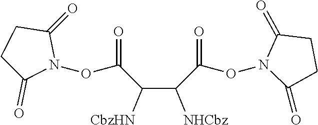 [SMILES: O=C(NC(C(=O)ON1C(=O)CCC1=O)C(NC(=O)OCc1ccccc1)C(=O)ON1C(=O)CCC1=O)OCc1ccccc1]

To a solution of 2,3-bis(((benzyloxy)carbonyl)amino)succinic acid (4.25 g, 10.21 mmol) in the mixture of DMA (70 ml) was added NHS (3.60 g, 31.30 mmol) and EDC (7.05 g, 36.72 mmol). The mixture was stirred for overnight, concentrated and purified on SiO₂ column eluted with EtOAc/CH₂Cl₂ (1:6) to afford the title compound (5.42 g, 87% yield, ˜95% pure). MS ESI m/z calcd for C₂₈H₂₇N₄O₁₂ [M+H]⁺ 611.15, found 611.60.

### Example 63. Synthesis of di-tert-butyl 4,4′-((2,3-bis(((benzyloxy)carbonyl)amino)-succinyl)bis(azanediyl))dibutanoate

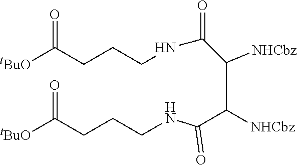 [SMILES: CC(C)(C)OC(=O)CCCNC(=O)C(NC(=O)OCc1ccccc1)C(NC(=O)OCc1ccccc1)C(=O)NCCCC(=O)OC(C)(C)C]

To a solution of 2,3-bis(((benzyloxy)carbonyl)amino)succinic acid (4.25 g, 10.21 mmol) in the mixture of DMA (70 ml) was added tert-butyl 4-aminobutanoate (3.25 g, 20.42 mmol) and EDC (7.01 g, 36.70 mmol). The mixture was stirred for overnight, concentrated and purified on SiO₂ column eluted with EtOAc/CH₂Cl₂ (1:10) to afford the title compound (6.56 g, 92% yield, ˜95% pure). MS ESI m/z calcd for C₃₆H₅₁N₄O₁₀ [M+H]⁺ 699.35, found 699.55.

### Example 64. Synthesis of di-tert-butyl 4,4′-((2,3-diaminosuccinyl)bis(azanediyl))-dibutanoate

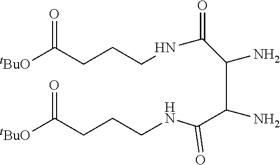 [SMILES: CC(C)(C)OC(=O)CCCNC(=O)C(N)C(N)C(=O)NCCCC(=O)OC(C)(C)C]

To a solution of di-tert-butyl 4,4′-((2,3-bis(((benzyloxy)carbonyl)amino)-succinyl)bis-(azanediyl))dibutanoate (2.50 g, 3.58 mmol) in MeOH (100 mL) was added 10% Pd/C (0.30 g, 50% wet), the mixture was stirred under hydrogen atmosphere at room temperature for 18 h. Then the Pd/C was removed by filtration through celite and the filter bed was washed with MeOH (˜70 ml). The filtrate was concentrated to afford the product as yellow foam which was used in the next step without further purification (1.55 g, 101% yield). ESI: m/z: calcd for C₂₀H₃₉N₂O₆ [M+H]⁺: 431.28, found 431.40.

### Example 65. Synthesis of di-tert-butyl 4,4′-((2,3-bis(3-(2,5-dioxo-2,5-dihydro-1H-pyrrol-1-yl)propanamido)succinyl)bis(azanediyl))dibutanoate

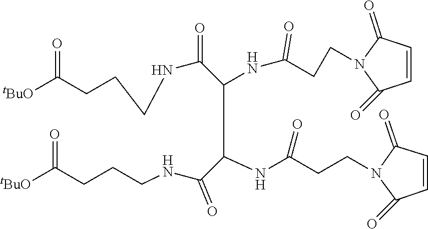 [SMILES: CC(C)(C)OC(=O)CCCNC(=O)C(NC(=O)CCN1C(=O)C=CC1=O)C(NC(=O)CCN1C(=O)C=CC1=O)C(=O)NCCCC(=O)OC(C)(C)C]

To a solution of 3-(2,5-dioxo-2,5-dihydro-1H-pyrrol-1-yl)propanoic acid (1.25 g, 7.39 mmol) in the mixture of DMA (60 ml) was added di-tert-butyl 4,4′-((2,3-diaminosuccinyl)bis(azanediyl))-dibutanoate (1.55 g, -3.58 mmol) and EDC (2.41 g, 12.61 mmol). The mixture was stirred for overnight, concentrated and purified on SiO₂ column eluted with EtOAc/CH₂Cl₂ (1:10) to afford the title compound (2.33 g, 89% yield). MS ESI m/z calcd for C₃₄H₄₉N₆O₁₂ [M+H]⁺ 733.33, found 733.50.

### Example 66. Synthesis of 4,4′-((2,3-bis(3-(2,5-dioxo-2,5-dihydro-1H-pyrrol-1-yl)propanamido)succinyl)bis(azanediyl))dibutanoic Acid

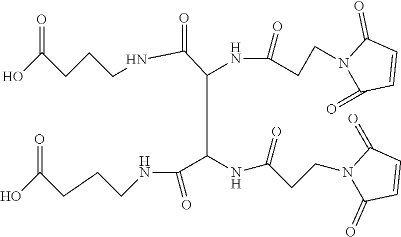 [SMILES: O=C(O)CCCNC(=O)C(NC(=O)CCN1C(=O)C=CC1=O)C(NC(=O)CCN1C(=O)C=CC1=O)C(=O)NCCCC(=O)O]

To a stirred solution of di-tert-butyl 4,4′-((2,3-bis(3-(2,5-dioxo-2,5-dihydro-1H-pyrrol-1-yl)propanamido)succinyl)bis(azanediyl))dibutanoate (2.30 g, 3.14 mmol) in 1,4-dioxane (20 ml) was added HCl (36%, 7.0 ml). The mixture was stirred for 30 min, diluted with toluene (20 ml), concentrated and purified on SiO₂ column eluted with MeOH/CH₂Cl₂ (1:10 to 1:4) to afford the title compound (1.67 g, 85% yield). MS ESI m/z calcd for C₂₆H₃₃N₆O₁₂ [M+H]⁺ 621.21, found 621.55.

### Example 67. Synthesis of di-tert-butyl 4,4′-((2,3-bis(2-(2,5-dioxo-2,5-dihydro-1H-pyrrol-1-yl)acetamido)succinyl)bis(azanediyl))dibutanoate

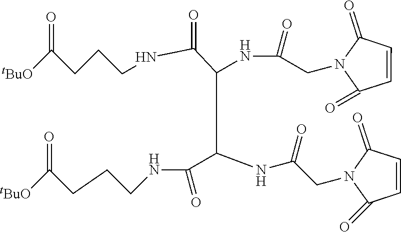 [SMILES: CC(C)(C)OC(=O)CCCNC(=O)C(NC(=O)CN1C(=O)C=CC1=O)C(NC(=O)CN1C(=O)C=CC1=O)C(=O)NCCCC(=O)OC(C)(C)C]

To a solution of 2-(2,5-dioxo-2,5-dihydro-1H-pyrrol-1-yl)acetic acid (1.12 g, 7.22 mmol) in the mixture of DMA (60 ml) was added di-tert-butyl 4,4′-((2,3-diaminosuccinyl)bis-(azanediyl))dibutanoate (1.55 g, -3.58 mmol) and EDC (2.40 g, 12.56 mmol). The mixture was stirred for overnight, concentrated and purified on SiO₂ column eluted with EtOAc/CH₂Cl₂ (1:10) to afford the title compound (2.27 g, 90% yield). MS ESI m/z calcd for C₃₂H₄₅N₆O₁₂ [M+H]⁺ 704.30, found 704.55.

### Example 68. Synthesis of 4,4′-((2,3-bis(2-(2,5-dioxo-2,5-dihydro-1H-pyrrol-1-yl)acetamido)succinyl)bis(azanediyl))dibutanoic Acid

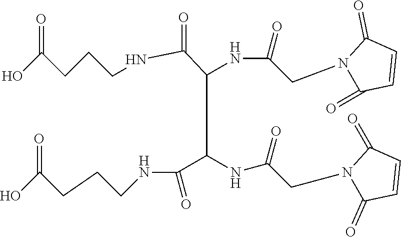 [SMILES: O=C(O)CCCNC(=O)C(NC(=O)CN1C(=O)C=CC1=O)C(NC(=O)CN1C(=O)C=CC1=O)C(=O)NCCCC(=O)O]

To a stirred solution of di-tert-butyl 4,4′-((2,3-bis(2-(2,5-dioxo-2,5-dihydro-1H-pyrrol-1-yl)acetamido)succinyl)bis(azanediyl))dibutanoate (2.20 g, 3.12 mmol) in 1,4-dioxane (20 ml) was added HCl (36%, 7.0 ml). The mixture was stirred for 30 min, diluted with toluene (20 ml), concentrated and purified on SiO₂ column eluted with MeOH/CH₂Cl₂ (1:10 to 1:4) to afford the title compound (1.67 g, 85% yield). MS ESI m/z calcd for C₂₄H₂₉N₆O₁₂ [M+H]⁺ 593.18, found 593.50.

### Example 69. Synthesis of bis(2,5-dioxopyrrolidin-1-yl) 4,4′-((2,3-bis(2-(2,5-dioxo-2,5-dihydro-1H-pyrrol-1-yl)acetamido)succinyl)bis(azanediyl))dibutanoate

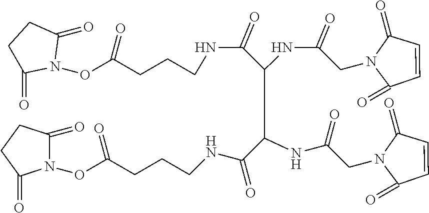 [SMILES: O=C(CN1C(=O)C=CC1=O)NC(C(=O)NCCCC(=O)ON1C(=O)CCC1=O)C(NC(=O)CN1C(=O)C=CC1=O)C(=O)NCCCC(=O)ON1C(=O)CCC1=O]

To a solution of 4,4′-((2,3-bis(2-(2,5-dioxo-2,5-dihydro-1H-pyrrol-1-yl)acetamido)-succinyl)bis(azanediyl))dibutanoic acid (1.10 g, 1.85 mmol) in the mixture of DMA (30 ml) was added NHS (1-hydroxypyrrolidine-2,5-dione) (0.55 g, 4.78 mmol) and EDC (1.25 g, 6.54 mmol). The mixture was stirred for overnight, concentrated and purified on SiO₂ column eluted with EtOAc/CH₂Cl₂ (1:10) to afford the title compound (1.28 g, 88% yield). MS ESI m/z calcd for C₃₂H₃₅N₈O₁₆ [M+H]⁺ 787.21, found 787.50.

### Example 70. Synthesis of 2,3-bis(2,5-dioxo-2,5-dihydro-1H-pyrrol-1-yl)succinic Acid

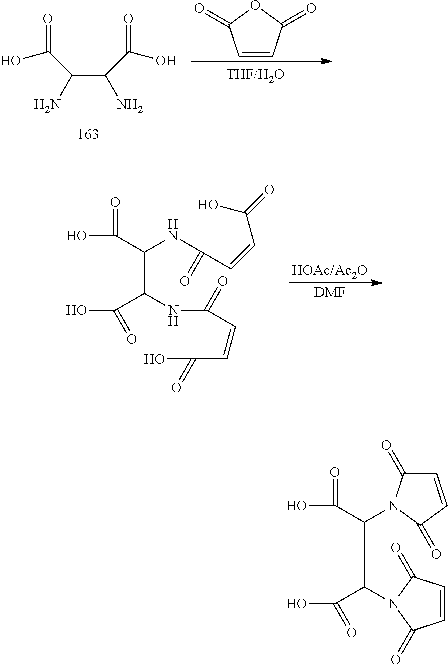

2,3-Diaminosuccinic acid (5.00 g, 33.77 mmol) in the mixture of THF/H₂O/DIPEA (125 ml/125 ml/2 ml) was added maleic anhydride (6.68 g, 68.21 mmol). The mixture was stirred for overnight, evaporated to afforded 2,3-bis((Z)-3-carboxyacrylamido)succinic acid (11.05 g, 99% yield) as a white solid. MS ESI m/z calcd for C12H₁₃N2010 [M+H]⁺ 345.05, found 345.35. 2,3-bis((Z)-3-carboxyacrylamido)succinic acid (11.05 g, 33.43 mmol) in a mixture solution of HOAc (70 ml), DMF (10 ml) and toluene (50 ml) was added acetic anhydride (30 ml). The mixture was stirred for 2 h, reflux with Dean-Stark Trap at 100° C. for 6 h, concentrated, co-evaporated with EtOH (2×40 ml) and toluene (2×40 ml), and purified on SiO₂ column eluted with H₂O/CH₃CN (1:10) to afford the title compound (7.90 g, 76% yield, ˜95% pure). MS ESI m/z calcd for Cl₂H₉N₂O₈[M+H]⁺ 309.03, found 309.30.

### Example 71. Synthesis of bis(2,5-dioxopyrrolidin-1-yl) 2,3-bis(2,5-dioxo-2,5-dihydro-1H-pyrrol-1-yl)succinate

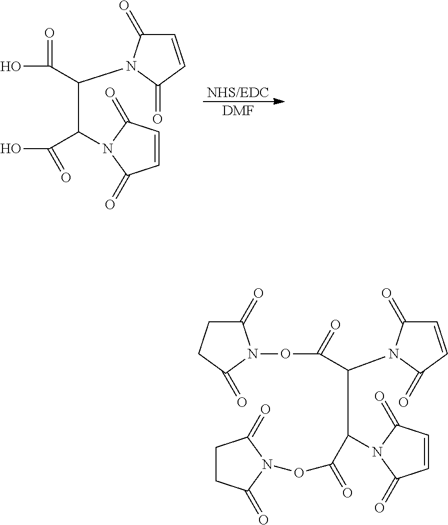

To a solution of 2,3-bis(2,5-dioxo-2,5-dihydro-1H-pyrrol-1-yl)succinic acid (4.00 g, 12.98 mmol) in the mixture of DMF (70 ml) was added NHS (3.60 g, 31.30 mmol) and EDC (7.05 g, 36.72 mmol). The mixture was stirred for overnight, concentrated and purified on SiO₂ column eluted with EtOAc/CH₂Cl₂ (1:6) to afford the title compound (5.73 g, 88% yield, ˜96% pure by HPLC). MS ESI m/z calcd for C₂₀H₁₅N₄O₁₂ [M+H]⁺ 503.06, found 503.45.

### Example 72. Synthesis of (3S,6S,39S,42S)-di-tert-butyl 6,39-bis(4-((tert-butoxycarbonyl)amino)butyl)-22,23-bis(2,5-dioxo-2,5-dihydro-1H-pyrrol-1-yl)-3,42-bis((4-(hydroxymethyl)phenyl)carbamoyl)-5,8,21,24,37,40-hexaoxo-11,14,17,28,31,34-hexaoxa-4,7,20,25,38,41-hexaazatetratetracontane-1,44-dioate

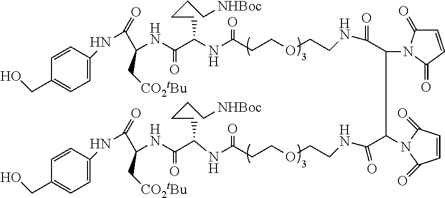

(14S,17S)-tert-butyl 1-amino-14-(4-((tert-butoxycarbonyl)amino)butyl)-17-((4-(hydroxymethyl)phenyl)carbamoyl)-12,15-dioxo-3,6,9-trioxa-13,16-diazanonadecan-19-oate (1.43 g, 1.97 mmol) and 2,3-bis(2,5-dioxo-2,5-dihydro-1H-pyrrol-1-yl)succinic acid (0.30 g, 0.97 mmol) in DMA (25 ml) was added EDC (1.30 g, 6.77 mmol). The mixture was stirred for overnight, evaporated in vacuo, purified on silica gel using a mixture of methanol (from 5% to 8%) in methylene chloride containing as the eluant to give title compound (1.33 g, 80% yield). ESI MS m/z C₈₂H₁₂₃N₁₂O₂₈ [M+H]⁺, cacld. 1722.85, found 1722.98.

### Example 73. Synthesis of tert-butyl 1-azido-14,17-dimethyl-12,15-dioxo-3,6,9-trioxa-13,16-diazaoctadecan-18-oate

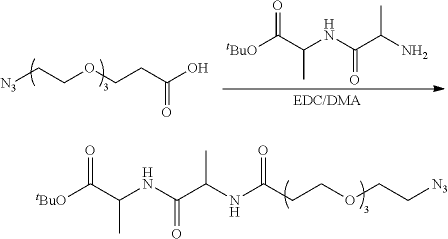

To a solution of 3-(2-(2-(2-azidoethoxy)ethoxy)ethoxy)propanoic acid (1.55 g, 6.27 mmol), tert-butyl 2-(2-aminopropanamido)propanoate (1.35 g, 6.27 mmol) in the mixture of DMA (60 ml) was added EDC (3.05 g, 15.88 mmol). The mixture was stirred for overnight, concentrated and purified on SiO₂ column eluted with EtOAc/CH₂Cl₂ (1:3) to afford the title compound (2.42 g, 86% yield, ˜95% pure by HPLC). MS ESI m/z calcd for C₁₉H₃₆N₅O₇ [M+H]⁺ 446.25, found 446.60.

### Example 74. Synthesis of 1-azido-14,17-dimethyl-12,15-dioxo-3,6,9-trioxa-13,16-diazaoctadecan-18-oic Acid

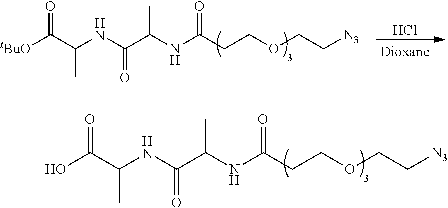

Tert-butyl 1-azido-14,17-dimethyl-12,15-dioxo-3,6,9-trioxa-13,16-diazaoctadecan-18-oate (2.20 g, 4.94 mmol) in 1,4-dioxane (40 ml) was added HCl (12 M, 10 ml). The mixture was stirred for 40 min, diluted with dioxane (20 ml) and toluene (40 ml), evaporated and co-evaporated with dioxane (20 ml) and toluene (40 ml) to dryness to afford the crude title product for the next step without further production (1.92 g, 100% yield, ˜94% pure by HPLC). MS ESI m/z calcd for C₁₅H₂₈N₅O₇[M+H]⁺ 390.19, found 390.45.

### Example 75. Synthesis of21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioic Acid

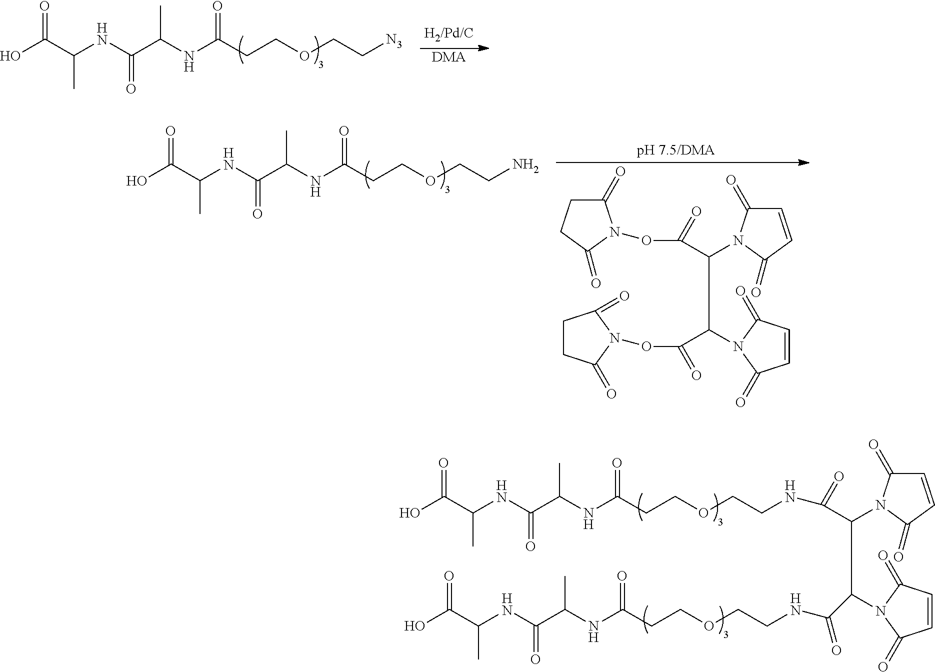

1-Azido-14,17-dimethyl-12,15-dioxo-3,6,9-trioxa-13,16-diazaoctadecan-18-oic acid (1.90 g, 4.88 mmol) in DMA (40 ml) was added Pd/C (0.20 g, 50% wet). The system was evacuated under vacuum and placed under 2 atm of hydrogen gas via hydrogenation reactor with vigorous stirring. The reaction was then stirred for 6 h at room temperature and TLC showed that the starting materials disappeared. The crude reaction was passed through a short pad of Celite rinsing with ethanol. The solvent was concentrated under reduced pressure to afford the crude product, 1-amino-14,17-dimethyl-12,15-dioxo-3,6,9-trioxa-13,16-diazaoctadecan-18-oic acid in DMA which was used for the next step directly. ESI MS m/z+C₁₅H₃₀N₃O₇ (M+H), cacld. 364.20, found 364.30.

To the amino compound in DMA (˜30 ml) was added 0.1 M NaH2PO4, pH 7.5 (20 ml), followed by addition of bis(2,5-dioxopyrrolidin-1-yl) 2,3-bis(2,5-dioxo-2,5-dihydro-1H-pyrrol-1-yl)succinate (1.30 g, 2.59 mmol). The mixture was stirred overnight, concentrated and purified on SiO₂ column eluted with 8% water on CH₃CN to afford the title compound (1.97 g, 81% yield). ESI MS m/z+C₄₂H₆₃N₈O₂₀ (M+H), cacld. 999.41, found 999.95.

### Example 76. Synthesis of bis(2,5-dioxopyrrolidin-1-yl) 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioate

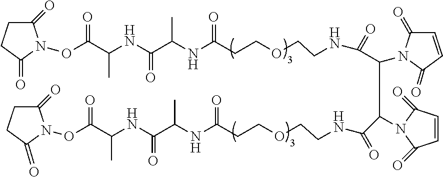

To a solution of 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioic acid (1.50 g, 1.50 mmol) in the mixture of DMA (10 ml) was added NHS (0.60 g, 5.21 mmol) and EDC (1.95 g, 10.15 mmol). The mixture was stirred for overnight, concentrated and purified on SiO₂ column eluted with EtOAc/CH₂Cl₂ (1:4 to 2:1) to afford the title compound (1.50 g, 83% yield, ˜95% pure by HPLC). MS ESI m/z calcd for C₅₀H₆₉N₁₀O₂₄ [M+H]⁺ 1193.44, found 1193.95.

### Example 77. Synthesis of (S)-tert-butyl 2-(hydroxymethyl)pyrrolidine-1-carboxylate

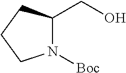 [SMILES: CC(C)(C)OC(=O)N1CCC[C@H]1CO]

Boc-L-proline (10.0 g, 46.4 mmol) dissolved in 50 mL THF was cooled to 0° C., to which BH₃ in THE (1.0 M, 46.4 mL) was added carefully. The mixture was stirred at 0° C. for 1.5 h then poured onto ice water and extracted with ethyl acetate. The organic layer was washed with brine (50 mL), dried over anhydrous Na₂SO₄, and concentrated under reduced pressure to give the title compound (8.50 g, 91% yield) as a white solid. ¹H NMR (500 MHz, CDCl₃) δ 3.94 (dd, J=4.9, 2.7 Hz, 2H), 3.60 (ddd, J=18.7, 11.9, 9.3 Hz, 2H), 3.49-3.37 (m, 1H), 3.34-3.23 (m, 1H), 2.06-1.91 (m, 1H), 1.89-1.69 (m, 2H), 1.65-1.51 (m, 1H), 1.49-0.40 (m, 9H).

### Example 78. Synthesis of (S)-tert-butyl 2-formylpyrrolidine-1-carboxylate

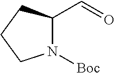 [SMILES: CC(C)(C)OC(=O)N1CCC[C@H]1C=O]

To a solution of (S)-tert-butyl 2-(hydroxymethyl)pyrrolidine-1-carboxylate (13.0 g, 64.6 mmol) in dimethyl sulfoxide (90 mL) was added triethylamine (40 mL) and the stirring was continued for 15 min. The mixture was cooled over ice bath and sulfur trioxide-pyridine complex (35.98 g, 226 mmol) was added in portions over a 40 min period. The reaction was warmed to r.t. and stirred for 2.5 h. After addition of ice (250 g), the mixture was extracted with dichloromethane (150 mL×3). The organic phase was washed with 50% citric acid solution (150 mL), water (150 mL), saturated sodium bicarbonate solution (150 mL), and brine (150 mL), dried over anhydrous Na2SO4. Removal of solvent in vacuo yielded the title aldehyde (10.4 g, 81% yield) as a dense oil which was used without further purification. ¹H NMR (500 MHz, CDCl₃) δ 9.45 (s, 1H), 4.04 (s, 1H), 3.53 (dd, J=14.4, 8.0 Hz, 2H), 2.00-1.82 (m, 4H), 1.44 (d, J=22.6 Hz, 9H).

### Example 79. Synthesis of (4R,5S)-4-methyl-5-phenyl-3-propionyloxazolidin-2-one

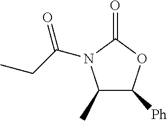 [SMILES: CCC(=O)N1C(=O)O[C@@H](c2ccccc2)[C@H]1C]

n-Butyllithium in hexane (21.6 mL, 2.2 M, 47.43 mmol) was added dropwise at −78° C. to a stirred solution of 4-methyl-5-phenyloxazolidin-2-one (8.0 g, 45.17 mmol) in THE (100 mL) under N₂. The solution was maintained at −78° C. for 1 h then propionyl chloride (4.4 mL, 50.59 mmol) was added slowly. The reaction mixture was warmed to −50° C., stirred for 2 h then quenched by addition of a saturated solution of ammonium chloride (100 mL). The organic solvent was removed in vacuo and the resultant solution was extracted with ethyl acetate (3×100 mL). The organic layer was washed with saturated sodium bicarbonate solution (100 mL) and brine (100 mL), dried over Na₂SO₄, filtered and concentrated in vacuo. The residue was purified by column chromatography (20% ethyl acetate/hexanes) to afford the title compound as a dense oil (10.5 g, 98% yield). 1H NMR (500 MHz, CDCl₃) δ 7.45-7.34 (m, 3H), 7.30 (d, J=7.0 Hz, 2H), 5.67 (d, J=7.3 Hz, 1H), 4.82-4.70 (m, 1H), 2.97 (dd, J=19.0, 7.4 Hz, 2H), 1.19 (t, J=7.4 Hz, 3H), 0.90 (d, J=6.6 Hz, 3H).

### Example 80. Synthesis of (S)-tert-butyl 2-((1R,2R)-1-hydroxy-2-methyl-3-((4R,5S)-4-methyl-2-oxo-5-phenyloxazolidin-3-yl)-3-oxopropyl)pyrrolidine-1-carboxylate

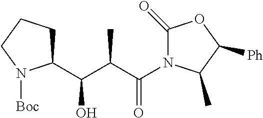 [SMILES: C[C@@H]1[C@H](c2ccccc2)OC(=O)N1C(=O)[C@H](C)[C@@H](O)[C@@H]1CCCN1C(=O)OC(C)(C)C]

To a solution of (4R,5S)-4-methyl-5-phenyl-3-propionyloxazolidin-2-one (9.40 g, 40.4 mmol) in dichloromethane (60 mL) was added Et₃N (6.45 mL, 46.64 mmol) at 0° C., followed by 1M dibutylboron triflate in dichloromethane (42 mL, 42 mmol). The mixture was stirred at 0° C. for 45 min, cooled to −70° C., (S)-tert-butyl 2-formylpyrrolidine-1-carboxylate (4.58 g, 22.97 mmol) in dichloromethane (40 mL) was then added slowly over a 30 min period. The reaction was stirred at −70° C. for 2 h, 0° C. 1 h, and r.t. 15 min, and then quenched with phosphate buffer solution (pH 7, 38 mL). After the addition of MeOH-30% H₂O₂(2:1, 100 mL) at below 10° C. and stirring for 20 min, water (100 mL) was added and the mixture was concentrated in vacuo. More water (200 mL) was added to the residue and the mixture was extracted with ethyl acetate (3×100 mL). The organic layer was washed with 1N KHSO₄ (100 mL), sodium bicarbonate solution (100 mL) and brine (100 mL), dried over anhydrous Na₂SO₄ and concentrated in vacuo. The residue was purified by flash column chromatography (10%-50% ethyl acetate/hexanes) to afford the title compound as a white solid (7.10 g, 71% yield). ¹H NMR (500 MHz, CDCl₃) δ 7.39 (dt, J=23.4, 7.1 Hz, 3H), 7.30 (d, J=7.5 Hz, 2H), 5.67 (d, J=7.1 Hz, 1H), 4.84-4.67 (m, 1H), 4.08-3.93 (m, 3H), 3.92-3.84 (m, 1H), 3.50 (d, J=9.0 Hz, 1H), 3.24 (d, J=6.7 Hz, 1H), 2.15 (s, 1H), 1.89 (dd, J=22.4, 14.8 Hz, 3H), 1.48 (d, J=21.5 Hz, 9H), 1.33 (d, J=6.9 Hz, 3H), 0.88 (d, J=6.4 Hz, 3H).

### Example 81. Synthesis of (S)-tert-butyl 2-((1R,2R)-1-methoxy-2-methyl-3-((4R,5S)-4-methyl-2-oxo-5-phenyloxazolidin-3-yl)-3-oxopropyl)pyrrolidine-1-carboxylate

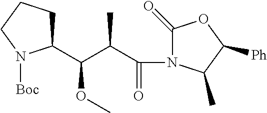 [SMILES: CO[C@H]([C@@H](C)C(=O)N1C(=O)O[C@@H](c2ccccc2)[C@H]1C)[C@@H]1CCCN1C(=O)OC(C)(C)C]

To a mixture of (S)-tert-butyl 2-((1R,2R)-1-hydroxy-2-methyl-3-((4R,5S)-4-methyl-2-oxo-5-phenyloxazolidin-3-yl)-3-oxopropyl)pyrrolidine-1-carboxylate (5.1 g 11.9 mmol) and molecular sieves (4 Å, 5 g) was added anhydrous dichloroethane (30 mL) under N₂. The mixture was stirred at room temperature for 20 min and cooled to 0° C. Proton sponge (6.62 g, 30.9 mmol) was added, followed by trimethyloxonium tetrafluoroborate (4.40 g, 29.7 mmol). Stirring was continued for 2 h at 0° C. and 48 h at r.t. The reaction mixture was filtrated and the filtrate was concentrated and purified by column chromatography (20-70% ethyl acetate/hexanes) to afford the title compound as a white solid (1.80 g, 35% yield). ¹H NMR (500 MHz, CDCl₃) δ 7.46-7.27 (m, 5H), 5.65 (s, 1H), 4.69 (s, 1H), 3.92 (s, 1H), 3.83 (s, 1H), 3.48 (s, 3H), 3.17 (s, 2H), 2.02-1.68 (m, 5H), 1.48 (d, J=22.3 Hz, 9H), 1.32 (t, J=6.0 Hz, 3H), 0.91-0.84 (m, 3H).

### Example 82. Synthesis of (2R,3R)-3-((S)-1-(tert-butoxycarbonyl)pyrrolidin-2-yl)-3-methoxy-2-methylpropanoic Acid

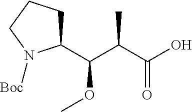 [SMILES: CO[C@H]([C@@H](C)C(=O)O)[C@@H]1CCCN1C(=O)OC(C)(C)C]

To a solution of (S)-tert-butyl 2-((1R,2R)-1-methoxy-2-methyl-3-((4R,5S)-4-methyl-2-oxo-5-phenyloxazolidin-3-yl)-3-oxopropyl)pyrrolidine-1-carboxylate (1.80 g, 4.03 mmol) in THE (30 mL) and H₂O (7.5 mL), 30% H₂O₂(1.44 mL, 14.4 mmol) was added over a 5 min period at 0° C., followed by a solution of LiOH (0.27 g, 6.45 mmol) in water (5 mL). After stirring at 0° C. for 3 h, 1 N sodium sulfite (15.7 mL) was added and the mixture was allowed to warm to r.t. and stirred overnight. THE was removed in vacuo and the aqueous phase was wash with dichloromethane (3×50 mL) to remove the oxazolidinone auxiliary. The aqueous phase was acidified to pH 3 with 1N HCl and extracted with ethyl acetate (3×50 mL). The organic layer was washed with brine (50 mL), dried over Na2SO4, filtered and concentrated in vacuo to afford the title compound as a colorless oil (1.15 g, 98% yield). ¹H NMR (500 MHz, CDCl₃) δ 3.99-3.74 (m, 2H), 3.44 (d, J=2.6 Hz, 3H), 3.23 (s, 1H), 2.60-2.45 (m, 1H), 1.92 (tt, J=56.0, 31.5 Hz, 3H), 1.79-1.69 (m, 1H), 1.58-1.39 (m, 9H), 1.30-1.24 (m, 3H).

### Example 83. Synthesis of (2R,3R)-methyl 3-methoxy-2-methyl-3-((S)-pyrrolidin-2-yl)propanoate

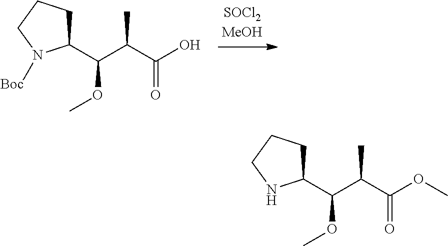 [SMILES: CO[C@H]([C@@H](C)C(=O)O)[C@@H]1CCCN1C(=O)OC(C)(C)C ; COC(=O)[C@H](C)[C@@H](OC)[C@@H]1CCCN1]

To a solution of (2R,3R)-3-((S)-1-(tert-butoxycarbonyl)pyrrolidin-2-yl)-3-methoxy-2-methylpropanoic acid. (0.86 g, 2.99 mmol) in MeOH (10 mL) was added thionyl chloride (1.08 mL, 14.95 mmol) slowly at 0° C. The reaction was then warmed to room temperature and stirred overnight. The mixture was concentrated in vacuo and co-evaporation with toluene giving the title compound (0.71 g, 100% yield) as a white solid, which was immediately used for the next step without further purification. HRMS (ESI) m/z calcd. for C₁₀H₂₀NO₃ [M+H]⁺: 202.14, found: 202.14.

### Example 84. Synthesis of (4S,5S)-ethyl 4-((tert-butoxycarbonyl)amino)-5-methyl-3-oxo heptanoate

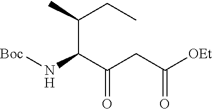 [SMILES: CCOC(=O)CC(=O)[C@@H](NC(=O)OC(C)(C)C)[C@@H](C)CC]

To an ice-cooled solution of N-Boc-L-isoleucine (4.55 g, 19.67 mmol) in THE (20 mL) was added 1,1′-carbonyldiimidazole (3.51 g, 21.63 mmol). After evolution of gas ceased, the resultant mixture was stirred at r.t. for 3.5 h.

A solution of freshly prepared isopropylmagnesium bromide in THE (123 mmol, 30 mL) was added dropwise to a pre-cooled (0° C.) solution of ethyl hydrogen malonate (6.50 g, 49.2 mmol) at such a rate to keep the internal temperature below 5° C. The mixture was stirred at r.t. for 1.5 h. This solution of the magnesium enolate was then cooled over an ice-water bath, followed by the gradual addition of the imidazolide solution over a 1 h period via a double-ended needle at 0° C. The resultant mixture was stirred at 0° C. for 30 min then r.t. 64 h. The reaction mixture was quenched by addition of 10% aqueous citric acid (5 mL), and acidified to pH 3 with an additional 10% aqueous citric acid (110 mL). The mixture was extracted with ethyl acetate (3×150 mL). The organic extracts were washed with water (50 mL), saturated aqueous sodium hydrogen carbonate (50 mL), and saturated aqueous sodium chloride (50 mL), dried over Na₂SO₄, and concentrated in vacuo. The residue was purified by column chromatography on silica gel using ethyl acetate/hexane (1:4) as an eluent to give the title compound (5.50 g, 93% yield). ¹H NMR (500 MHz, CDCl₃) δ 5.04 (d, J=7.8 Hz, 1H), 4.20 (p, J=7.0 Hz, 3H), 3.52 (t, J=10.7 Hz, 2H), 1.96 (d, J=3.7 Hz, 1H), 1.69 (s, 2H), 1.44 (s, 9H), 1.28 (dd, J=7.1, 2.9 Hz, 3H), 0.98 (t, J=6.9 Hz, 3H), 0.92-0.86 (m, 3H).

### Example 85. Synthesis of (3R,4S,5S)-ethyl 4-((tert-butoxycarbonyl)amino)-3-hydroxy-5-methylheptanoate

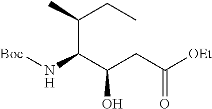 [SMILES: CCOC(=O)C[C@@H](O)[C@@H](NC(=O)OC(C)(C)C)[C@@H](C)CC]

To a solution of (4S,5S)-ethyl 4-((tert-butoxycarbonyl)amino)-5-methyl-3-oxo heptanoate (5.90 g, 19.83 mmol) in ethanol (6 mL) at −60° C. was added sodium borohydride (3.77 g, 99.2 mmol) in one portion. The reaction mixture was stirred for 5.5 h below −55° C. then quenched with 10% aqueous citric acid (100 mL). The resultant solution was acidified to pH 2 with an additional 10% aqueous citric acid, followed by extraction with ethyl acetate (3×100 mL). The organic extracts were washed with saturated aqueous sodium chloride (100 mL), dried over Na₂SO₄, and concentrated in vacuo. The residue was purified by column chromatography (10-50% ethyl acetate/hexane) to give pure the title compound as diastereomer (2.20 g, 37% yield) and a mixture of two diastereomers (2.0 g, 34% yield, about 9:1 ratio). ¹H NMR (500 MHz, CDCl₃) δ 4.41 (d, J=9.3 Hz, 1H), 4.17 (tt, J=7.1, 3.6 Hz, 2H), 4.00 (t, J=6.9 Hz, 1H), 3.55 (dd, J=11.7, 9.3 Hz, 1H), 2.56-2.51 (m, 2H), 2.44 (dd, J=16.4, 9.0 Hz, 1H), 1.79 (d, J=3.8 Hz, 1H), 1.60-1.53 (m, 1H), 1.43 (s, 9H), 1.27 (dd, J=9.3, 5.0 Hz, 3H), 1.03-0.91 (m, 7H).

### Example 86. Synthesis of (3R,4S,5S)-4-((tert-butoxycarbonyl)amino)-3-hydroxy-5-methyl heptanoic Acid

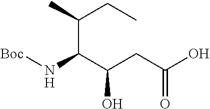 [SMILES: CC[C@H](C)[C@H](NC(=O)OC(C)(C)C)[C@H](O)CC(=O)O]

To a solution of (3R,4S,5S)-ethyl 4-((tert-butoxycarbonyl)amino)-3-hydroxy-5-methylheptanoate (2.20 g, 7.20 mmol) in ethanol (22 mL) was added 1 N aqueous sodium hydroxide (7.57 mL, 7.57 mmol). The mixture was stirred at 0° C. for 30 min then r.t. 2 h. The resultant solution was acidified to pH 4 by addition of 1 N aqueous hydrochloric acid, which was then extracted with ethyl acetate (3×50 mL). The organic extracts were washed with 1 N aqueous potassium hydrogen sulfate (50 mL), and saturated aqueous sodium chloride (50 mL), dried over Na₂SO₄, and concentrated in vacuo to give the compound (1.90 g, 95% yield). ¹H NMR (500 MHz, CDCl₃) δ 4.50 (d, J=8.7 Hz, 1H), 4.07 (d, J=5.5 Hz, 1H), 3.59 (d, J=8.3 Hz, 1H), 2.56-2.45 (m, 2H), 1.76-1.65 (m, 1H), 1.56 (d, J=7.1 Hz, 1H), 1.45 (s, 9H), 1.26 (t, J=7.1 Hz, 3H), 0.93 (dd, J=14.4, 7.1 Hz, 6H).

### Example 87. Synthesis of (3R,4S,5S)-4-((tert-butoxycarbonyl)(methyl)amino)-3-methoxy-5-methylheptanoic Acid

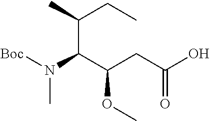 [SMILES: CC[C@H](C)[C@@H]([C@@H](CC(=O)O)OC)N(C)C(=O)OC(C)(C)C]

To a solution of (3R,4S,5S)-4-((tert-butoxycarbonyl)amino)-3-hydroxy-5-methyl heptanoic acid (1.90 g, 6.9 mmol) in THE (40 mL) was added sodium hydride (60% oil suspension, 1.93 g, 48.3 mmol) at 0° C. After stirring for 1h, methyl iodide (6.6 mL, 103.5 mmol) was added. The stirring was continued at 0° C. for 40 h before saturated aqueous sodium hydrogen carbonate (50 mL) was added, followed by water (100 mL). The mixture was washed with diethyl ether (2×50 mL) and the aqueous layer was acidified to pH 3 by 1 N aqueous potassium hydrogen sulfate, then extracted with ethyl acetate (3×50 mL). The combined organic extracts were washed with 5% aqueous sodium thiosulfate (50 mL) and saturated aqueous sodium chloride (50 mL), dried over Na₂SO₄, and concentrated in vacuo to give the title compound (1.00 g, 48% yield). ¹H NMR (500 MHz, CDCl₃) δ 3.95 (d, J=75.4 Hz, 2H), 3.42 (d, J=4.4 Hz, 3H), 2.71 (s, 3H), 2.62 (s, 1H), 2.56-2.47 (m, 2H), 1.79 (s, 1H), 1.47 (s, 1H), 1.45 (d, J=3.3 Hz, 9H), 1.13-1.05 (m, 1H), 0.96 (d, J=6.7 Hz, 3H), 0.89 (td, J=7.2, 2.5 Hz, 3H).

### Example 88. Synthesis of Boc-N-Me-L-Val-OH

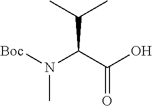 [SMILES: CC(C)[C@@H](C(=O)O)N(C)C(=O)OC(C)(C)C]

To a solution of Boc-L-Val-OH (2.00 g, 9.2 mmol) and methyl iodide (5.74 mL, 92 mmol) in anhydrous THF (40 mL) was added sodium hydride (3.68 g, 92 mmol) at 0° C. The reaction mixture was stirred at 0° C. for 1.5 h, then warmed to r.t. and stirred for 24 h. The reaction was quenched by ice water (50 mL). After addition of water (100 mL), the reaction mixture was washed with ethyl acetate (3×50 mL) and the aqueous solution was acidified to pH 3 then extracted with ethyl acetate (3×50 mL). The combined organic phase was dried over Na₂SO₄ and concentrated to afford Boc-N-Me-Val-OH (2.00 g, 94% yield) as a white solid. ¹H NMR (500 MHz, CDCl₃) δ 4.10 (d, J=10.0 Hz, 1H), 2.87 (s, 3H), 2.37-2.13 (m, 1H), 1.44 (d, J=26.7 Hz, 9H), 1.02 (d, J=6.5 Hz, 3H), 0.90 (t, J=8.6 Hz, 3H).

### Example 89. Synthesis of (2R,3R)-methyl 3-((S)-1-((3R,4S,5S)-4-((tert-butoxycarbonyl)-(methyl)amino)-3-methoxy-5-methylheptanoyl)pyrrolidin-2-yl)-3-methoxy-2-methylpropanoate

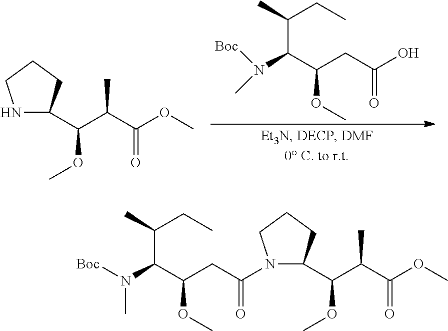 [SMILES: COC(=O)[C@H](C)[C@@H](OC)[C@@H]1CCCN1 ; CC[C@H](C)[C@@H]([C@@H](CC(=O)O)OC)N(C)C(=O)OC(C)(C)C ; CC[C@H](C)[C@@H]([C@@H](CC(=O)N1CCC[C@H]1[C@H](OC)[C@@H](C)C(=O)OC)OC)N(C)C(=O)OC(C)(C)C]

To a solution of (2R,3R)-methyl 3-methoxy-2-methyl-3-((S)-pyrrolidin-2-yl)propanoate (0.71 g, 2.99 mmol) and (3R,4S,5S)-4-((tert-butoxycarbonyl)(methyl)amino)-3-methoxy-5-methylheptanoic acid (1 g, 3.29 mmol) in DMF (10 mL) at 0° C. was added diethyl cyanophosphonate (545 μL, 3.59 mmol), followed by addition of Et₃N (1.25 mL, 8.99 mmol). The reaction mixture was stirred at 0° C. for 2h, then warmed to room temperature and stirred overnight. The reaction mixture was diluted with ethyl acetate (50 mL), washed with 1 N aqueous potassium hydrogen sulfate (20 mL), water (20 mL), saturated aqueous sodium hydrogen carbonate (20 mL), and saturated aqueous sodium chloride (20 mL), dried over sodium sulfate, and concentrated in vacuo. The residue was purified on silica gel column chromatography eluted with ethyl acetate/hexane (1:5 to 2:1) to afford the title (0.9 g, 62% yield) as a white solid. HRMS (ESI) m/z calcd. for C₂₅H₄₆N₂O₇ [M+H]⁺: 487.33, found: 487.32.

### Example 90. Synthesis of (S)-tert-butyl 2-((1R,2R)-1-methoxy-3-(((S)-1-methoxy-1-oxo-3-phenylpropan-2-yl)amino)-2-methyl-3-oxopropyl)pyrrolidine-1-carboxylate

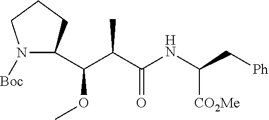 [SMILES: COC(=O)[C@H](Cc1ccccc1)NC(=O)[C@H](C)[C@@H](OC)[C@@H]1CCCN1C(=O)OC(C)(C)C]

To a solution of (2R,3R)-3-((S)-1-(tert-butoxycarbonyl)pyrrolidin-2-yl) -3-methoxy-2-methylpropanoic acid (100 mg, 0.347 mmol) and L-phenylalanine methyl ester hydrochloride (107.8 mg, 0.500 mmol) in DMF (5 mL) at 0° C. was added diethyl cyanophosphonate (75.6 μL, 0.451 mmol), followed by Et₃N (131 μL, 0.94 mmol). The reaction mixture was stirred at 0° C. for 2 h, then warmed to r.t. and stirred overnight. The reaction mixture was then diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over Na₂SO₄, and concentrated in vacuo. The residue was purified by column chromatography (15-75% ethyl acetate/hexanes) to afford the title compound (130 mg, 83% yield) as a white solid. ¹H NMR (500 MHz, CDCl₃) δ 7.28 (dd, J=7.9, 6.5 Hz, 2H), 7.23 (t, J=7.3 Hz, 1H), 7.16 (s, 2H), 4.81 (s, 1H), 3.98-3.56 (m, 5H), 3.50 (s, 1H), 3.37 (d, J=2.9 Hz, 3H), 3.17 (dd, J=13.9, 5.4 Hz, 2H), 3.04 (dd, J=14.0, 7.7 Hz, 1H), 2.34 (s, 1H), 1.81-1.69 (m, 2H), 1.65 (s, 3H), 1.51-1.40 (m, 9H), 1.16 (d, J=7.0 Hz, 3H).

### Example 91. General Procedure for the Removal of the Boc Function with Trifluoroacetic Acid

To a solution of the N-Boc amino acid (1.0 mmol) in methylene chloride (2.5 mL) was added trifluoroacetic acid (1.0 mL). After being stirred at room temperature for 1-3 h, the reaction mixture was concentrated in vacuo. Co-evaporation with toluene gave the deprotected product, which was used without any further purification.

### Example 92. Synthesis of (2R,3R)-methyl 3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanoate

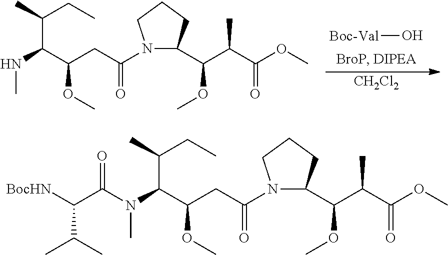

To a solution of the deprotected product from (2R,3R)-methyl 3-methoxy-3-((S)-1-((3R,4S,5S)-3-methoxy-5-methyl-4-(methylamino)heptanoyl)pyrrolidin-2-yl)-2-methylpropanoate (715 mg, 1.85 mmol) and Boc-Val-OH (1.2 g, 5.56 mmol) in DCM (20 mL) at 0° C. was added BroP (1.08 g, 2.78 mmol), followed by addition of diisopropylethylamine (1.13 mL, 6.48 mmol). The mixture was shielded from light and stirred at 0° C. for 30 min then at r.t. for 48h. The reaction mixture was diluted with ethyl acetate (50 mL), washed with 1 N aqueous potassium hydrogen sulfate (20 mL), water (20 mL), saturated aqueous sodium hydrogen carbonate (20 mL), and saturated aqueous sodium chloride (20 mL), dried over Na₂SO₄ and concentrated in vacuo. The residue was purified on silica gel column chromatography eluted with ethyl acetate/hexane (1:5 to 4:1) to afford the title compound (0.92 g, 85% yield) as a white solid. HRMS (ESI) m/z calcd. for C₃₀H₅₅N₃O₈ [M+H]⁺: 586.40, found: 586.37.

### Example 93. Synthesis of (2R,3R)-methyl 3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoate

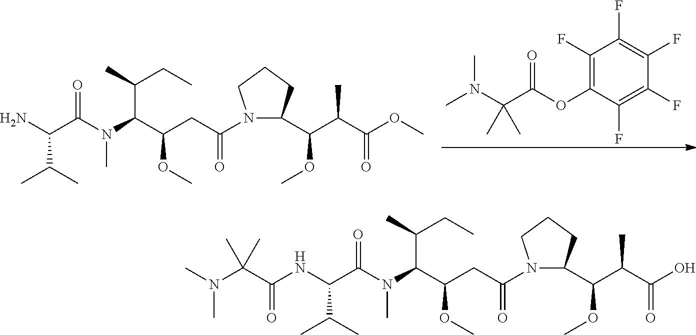

To a solution of the deprotected product from (2R,3R)-methyl 3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanoate (50 mg, 0.085 mmol) and perfluorophenyl 2-(dimethylamino)-2-methylpropanoate (74.5 mg, 0.25 mmol) in DMF (2 ml) at 0° C. was added DIPEA (44 μL, 0.255 mmol). The reaction mixture was warmed to RT and stirred 2h. The reaction mixture was diluted with ethyl acetate (30 mL), washed with water (10 mL), and saturated aqueous sodium chloride (10 mL), dried over sodium sulfate, and concentrated in vacuo. The residue was purified on silica gel column chromatography eluted with ethyl acetate/hexane (1:5 to 5:1) to afford the title compound (50 mg, 100% yield). HRMS (ESI) m/z calcd. for C₃₁H₅₈N₄O₇ [M+H]⁺: 599, found: 599.

### Example 94. Synthesis of (2R,3R)-3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoic Acid

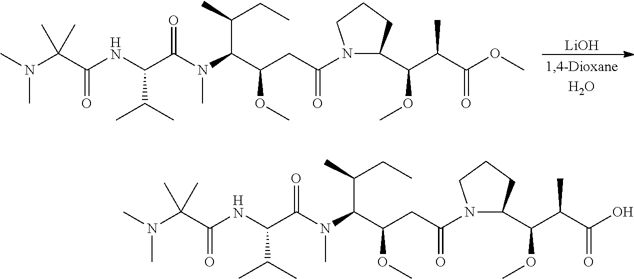

To a solution of (2R,3R)-methyl 3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoate (50 mg, 0.0836 mmol) in 1,4-Dioxane (3 mL) at 0-4° C. was added a solution of lithium hydroxide (14 mg, 0.334 mmol) in water (3 mL) drop by drop in 5 min. The reaction mixture was warmed to RT and stirred 2h. The mixture was acidified to pH 7 with 1N HCl and concentrated under vacuum, and then used for the next step without further purification. HRMS (ESI) m/z calcd. for C₃₀H₅₇N₄O₇ [M+H]⁺: 585.41, found: 585.80.

### Example 95. Synthesis of (2R,3R)-perfluorophenyl 3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoate

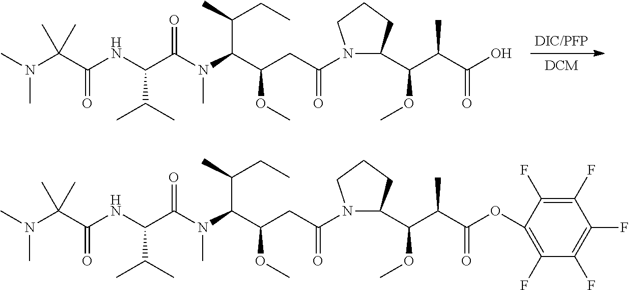

To a solution of (2R,3R)-3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoic acid (0.0836 mmol) and PFP (18.5 mg, 0.1 mmol) in DCM (2 mL) was added DIC (12.7 mg, 0.1 mmol) at 0° C. The mixture was warmed to RT and stirred overnight. The reaction mixture was concentrated under vacuum and used for the next step without further purification. HRMS (ESI) m/z calcd. for C₃₆H₅₆F5N₄O₇[M+H]⁺: 751.40, found: 751.70.

### Example 96. Synthesis of (S)-methyl 2-((tert-butoxycarbonyl)amino)-3-(4-hydroxy-3-nitrophenyl)propanoate

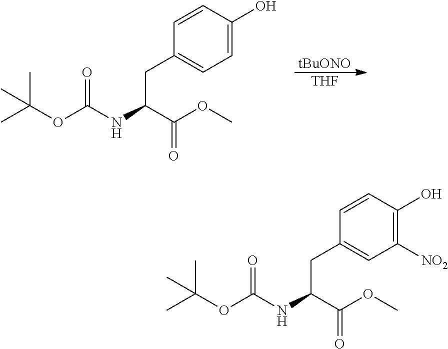 [SMILES: "tBuONO" ; COC(=O)[C@H](Cc1ccc(O)cc1)NC(=O)OC(C)(C)C ; COC(=O)[C@H](Cc1ccc(O)c([N+](=O)[O-])c1)NC(=O)OC(C)(C)C ; "THF"]

To a solution of Boc-L-Tyrosine methyl ester (5 g, 16.9 mmol) in THE (50 mL) was added tert-Butyl nitrite (10 mL, 84.6 mmol), then the reaction mixture was stirred for 5h at RT. The reaction mixture was concentrated and purified by column chromatography on silica gel using ethyl acetate/hexane (1:10 to 1:5) to afford the compound (4.5 g, 78% yield) as a yellow solid. HRMS (ESI) m/z calcd. for C₁₅H₂₁N₂O₇ [M+H]⁺: 341.13, found: 341.30.

### Example 97. Synthesis of (S)-methyl 3-(3-amino-4-hydroxyphenyl)-2-((tert-butoxycarbonyl)amino)propanoate

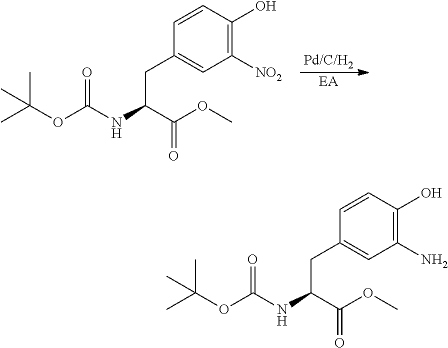 [SMILES: COC(=O)[C@H](Cc1ccc(O)c([N+](=O)[O-])c1)NC(=O)OC(C)(C)C ; "Pd/C/H2" ; "EA" ; COC(=O)[C@H](Cc1ccc(O)c(N)c1)NC(=O)OC(C)(C)C]

To a solution of (S)-methyl 3-(3-amino-4-hydroxyphenyl)-2-(tert-butoxycarbonylamino)propanoate (2 g, 6.44 mmol) in ethyl acetate (20 mL) was added Pd/C (0.2 g) and stirred for 2h under hydrogen atmosphere. The mixture was filtered and the filtrate was concentrated under vacuum to afford the title compound (1.7 g, 95% yield) as a white solid. HRMS (ESI) m/z calcd. for C₁₅H₂₃N₂O₅ [M+H]⁺: 311.15, found: 311.30.

### Example 98. Synthesis of (2S)-methyl 3-(8,9-bis(3-(2,5-dioxo-2,5-dihydro-1H-pyrrol-1-yl)propanamido)-2,7,10,15-tetraoxo-3,4,5,6,7,8,9,10,11,12,13,14,15,16-tetradecahydro-2H-benzo[b][1,4,9,14]oxatriazacyclooctadecin-18-yl)-2-((tert-butoxycarbonyl)amino)propanoate

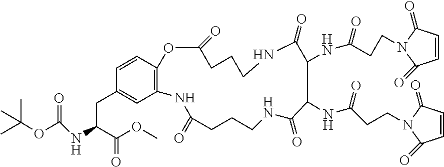 [SMILES: COC(=O)[C@H](Cc1ccc2c(c1)NC(=O)CCCNC(=O)C(NC(=O)CCN1C(=O)C=CC1=O)C(NC(=O)CCN1C(=O)C=CC1=O)C(=O)NCCCC(=O)O2)NC(=O)OC(C)(C)C]

To a solution of4,4′-((2,3-bis(2-(2,5-dioxo-2,5-dihydro-1H-pyrrol-1-yl)acetamido)-succinyl)bis(azanediyl))dibutanoic acid (108.0 mg, 0.182 mmol) and (S)-methyl 3-(3-amino-4-hydroxyphenyl)-2-(tert-butoxycarbonylamino)propanoate (56.6 mg, 0.182 mmol) in DMF (5 mL) at 0° C. was added EDC (130 mg, 0.678 mmol), followed by addition of DIPEA (64 μL, 0.365 mmol). The reaction mixture was warmed to RT and stirred overnight. The mixture was diluted with ethyl acetate (30 mL), washed with water (10 mL) and saturated aqueous sodium chloride (10 mL), dried over sodium sulfate and concentrated in vacuo. The residue was purified on silica gel column chromatography eluted with DCM/MeOH (20:1 to 10:1) to afford the title compound (110.6 mg, 68% yield). HRMS (ESI) m/z calcd. for C₄₁H₅₁N₈O₁₅ [M+H]⁺: 895.34, found: 895.30.

### Example 99. Synthesis of (2S)-methyl 2-amino-3-(8,9-bis(3-(2,5-dioxo-2,5-dihydro-1H-pyrrol-1-yl)propanamido)-2,7,10,15-tetraoxo-3,4,5,6,7,8,9,10,11,12,13,14,15,16-tetradecahydro-2H-benzo[b][1,4,9,14]oxatriazacyclooctadecin-18-yl)propanoate

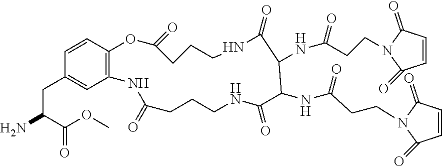 [SMILES: COC(=O)[C@@H](N)Cc1ccc2c(c1)NC(=O)CCCNC(=O)C(NC(=O)CCN1C(=O)C=CC1=O)C(NC(=O)CCN1C(=O)C=CC1=O)C(=O)NCCCC(=O)O2]

To a solution of (2S)-methyl 3-(8,9-bis(3-(2,5-dioxo-2,5-dihydro-1H-pyrrol-1-yl)propanamido)-2,7,10,15-tetraoxo-3,4,5,6,7,8,9,10,11,12,13,14,15,16-tetradecahydro-2H-benzo[b][1,4,9,14]oxatriazacyclooctadecin-18-yl)-2-((tert-butoxycarbonyl)amino)propanoate (100.2 mg, 0.112 mmol) in DCM (6 mL) was added TFA (2 mL) at 0° C. The reaction mixture was warmed to RT and stirred 30 min., diluted with toluene, concentrated, co-evaporated with toluene, and then used for the next step without further purification. HRMS (ESI) m/z calcd. for C₃₆H₄₃N₈O₁₃ [M+H]⁺: 795.29, found: 795.45.

### Example 100. Synthesis of (2S)-methyl 3-(8,9-bis(3-(2,5-dioxo-2,5-dihydro-1H-pyrrol-1-yl)propanamido)-2,7,10,15-tetraoxo-3,4,5,6,7,8,9,10,11,12,13,14,15,16-tetradecahydro-2H-benzo[b][1,4,9,14]oxatriazacyclooctadecin-18-yl)-2-((2R,3R)-3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanamido)propanoate (A-01)

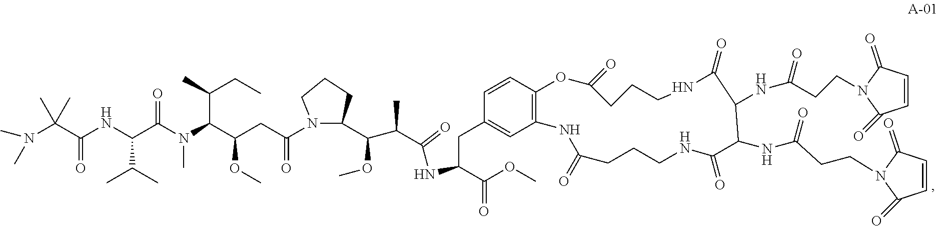 [SMILES: CC[C@H](C)[C@@H]([C@@H](CC(=O)N1CCC[C@H]1[C@H](OC)[C@@H](C)C(=O)N[C@@H](Cc1ccc2c(c1)NC(=O)CCCNC(=O)C(NC(=O)CCN1C(=O)C=CC1=O)C(NC(=O)CCN1C(=O)C=CC1=O)C(=O)NCCCC(=O)O2)C(=O)OC)OC)N(C)C(=O)[C@@H](NC(=O)C(C)(C)N(C)C)C(C)C]

To a solution of (2R,3R)-perfluorophenyl 3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-2-methylpropanoate (20 mg, 0.027 mmol) and (2S)-methyl 2-amino-3-(8,9-bis(3-(2,5-dioxo-2,5-dihydro-1H-pyrrol-1-yl)propanamido)-2,7,10,15-tetraoxo-3,4,5,6,7,8,9,10,11,12,13,14,15,16-tetradecahydro-2H-benzo[b][1,4,9,14]oxatriaza-cyclooctadecin-18-yl)propanoate (31.7 mg, 0.04 mmol) in DMA (2 mL) was added DIPEA (9 μL, 0.053 mmol) at 0° C. The reaction mixture was warmed to RT and stirred for 30 min. The mixture was concentrated under vacuum and purified by prep-HPLC (C-18, 250 mm×10 mm, eluted with H₂O/CH₃CN (9 ml/min, from 90% water to 40% water in 40 min) to afford the title compound (16 mg, 43% yield). HRMS (ESI) m/z calcd. for C₆₆H₉₇N₁₂O₁₉ [M+H]⁺: 1361.69 found: 1361.50.

### Example 101. Synthesis of (S)-methyl 2-((2R,3R)-3-((S)-1-((3R,4S,5S)-4-((tert-butoxycarbonyl)(methyl)amino)-3-methoxy-5-methylheptanoyl)pyrrolidin-2-yl)-3-methoxy-2-methylpropanamido)-3-phenylpropanoate

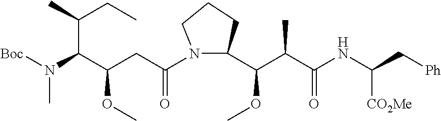 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)OC(C)(C)C]

To a solution of the Boc-deprotected product of (S)-tert-butyl 2-((1R,2R)-1-methoxy-3-(((S)-1-methoxy-1-oxo-3-phenylpropan-2-yl)amino)-2-methyl-3-oxopropyl)pyrrolidine-1-carboxylate (0.29 mmol) and (3R,4S,5S)-4-((tert-butoxycarbonyl)(methyl)amino)-3-methoxy-5-methylheptanoic acid (96.6 mg, 0.318 mmol) in DMF (5 mL) at 0° C. was added diethyl cyanophosphonate (58 μL, 0.347 mmol), followed by Et₃N (109 μL, 0.78 mmol). The reaction mixture was stirred at 0° C. for 2 h, then warmed to r.t. and stirred overnight. The reaction mixture was diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over Na2SO₄ and concentrated in vacuo. The residue was purified by column chromatography (15-75% ethyl acetate/hexanes) to afford the title compound (150 mg, 81% yield) as a white solid. LC-MS (ESI) m/z calcd. for C₃₄H₅₅N₃O₈ [M+H]⁺: 634.40, found: 634.40.

### Example 102. Synthesis of (S)-methyl 2-((2R,3R)-3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanamido)-3-phenylpropanoate

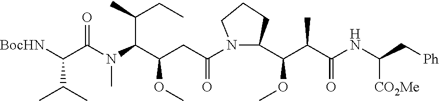 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)OC(C)(C)C)C(C)C]

To a solution of the Boc-deprotected product of (S)-methyl 2-((2R,3R)-3-((S)-1-((3R,4S,5S)-4-((tert-butoxycarbonyl)(methyl)amino)-3-methoxy-5-methylheptanoyl)-pyrrolidin-2-yl)-3-methoxy-2-methylpropanamido)-3-phenylpropanoate (0.118 mmol) and Boc-Val-OH (51.8 mg, 0.236 mmol) in DCM (5 mL) at 0° C. was added BroP (70.1 mg, 0.184 mmol), followed by diisopropylethylamine (70 μL, 0.425 mmol). The mixture was shielded from light and stirred at 0° C. for 30 min then at r.t. for 2 days. The reaction mixture was diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over Na2SO4 and concentrated in vacuo. The residue was purified by column chromatography (20-100% ethyl acetate/hexanes) to afford the title compound (67 mg, 77% yield) as a white solid. LC-MS (ESI) m/z calcd. for C₃₉H₆₄N₄O₉ [M+H]⁺: 733.47, found: 733.46.

### Example 103. Synthesis of (S)-methyl 2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapentadecan-15-oyl)pyrrolidin-2-yl)-3-methoxy-2-methylpropanamido)-3-phenylpropanoate

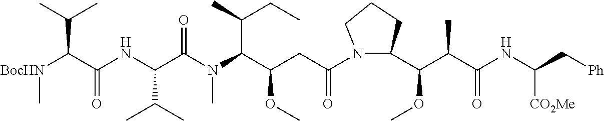

To a solution of the Boc-deprotected product of (S)-methyl 2-((2R,3R)-3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanamido)-3-phenylpropanoate (0.091 mmol) and Boc-N-Me-Val-OH (127 mg, 0.548 mmol) in DMF (5 mL) at 0° C. was added diethyl cyanophosphonate (18.2 μL, 0.114 mmol), followed by N-methylmorpholine (59 μL, 0.548 mmol). The reaction mixture was stirred at 0° C. for 2 h, then warmed to r.t. and stirred overnight. The reaction mixture was diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over sodium sulfate, and concentrated in vacuo. The residue was purified by column chromatography (20-100% ethyl acetate/hexanes) to afford the title compound (30 mg, 39% yield) as a white solid. LC-MS (ESI) m/z calcd. for C₄₅H₇₅N₅O₁₀ [M+H]⁺: 846.55, found: 846.56.

### Example 104. Synthesis of (S)-methyl 2-((2R,3R)-3-((S)-1-((3R,4S,5S)-4-((S)—N,3-dimethyl-2-((S)-3-methyl-2-(methylamino)butanamido)butanamido)-3-methoxy-5-methyl-heptanoyl)pyrrolidin-2-yl)-3-methoxy-2-methylpropanamido)-3-phenylpropanoate

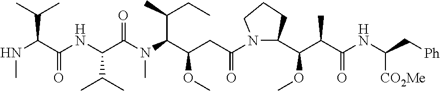 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@@H](NC)C(C)C)C(C)C]

To a solution of (S)-methyl 2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapentadecan-15-oyl)pyrrolidin-2-yl)-3-methoxy-2-methylpropanamido)-3-phenylpropanoate (75.0 mg, 0.0886 mmol) in methylene chloride (5 mL) was added trifluoroacetic acid (2 mL) at room temperature. After being stirred at room temperature for 1 h, the reaction mixture was concentrated in vacuo. Co-evaporation with toluene gave the deprotected title product, which was used without further purification.

### Example 105. Synthesis of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-pyrrolidin-2-yl)-3-methoxy-2-methylpropanamido)-3-phenylpropanoic Acid

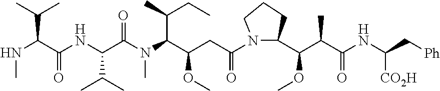 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

(S)-Methyl 2-((2R,3R)-3-((S)-1-((3R,4S,5S)-4-((S)—N,3-dimethyl-2-((S)-3-methyl-2-(methylamino)butanamido)butanamido)-3-methoxy-5-methyl-heptanoyl)pyrrolidin-2-yl)-3-methoxy-2-methylpropanamido)-3-phenylpropanoate (25 mg, 0.030 mmol) in the mixture of conc. HCl (0.3 ml) and 1,4-dioxane (0.9 ml) was stirred at r.t. for 35 min. The mixture was diluted with EtOH (1.0 ml) and toluene (1.0 ml), concentrated and co-evaporated with EtOH/toluene (2:1) to afford the title compound as a white solid (22 mg, ˜100% yield), which was used in the next step without further purification. LC-MS (ESI) m/z calcd. for C₃₉H₆₆N₅O₈ [M+H]⁺: 732.48, found: 732.60.

### Example 106. Synthesis of (2S)-2-((2R,3R)-3-((2S)-1-((11S,14S,17S)-1-azido-17-((R)-sec-butyl)-11,14-diisopropyl-18-methoxy-10,16-dimethyl-9,12,15-trioxo-3,6-dioxa-10,13,16-triazaicosan-20-oyl)pyrrolidin-2-yl)-3-methoxy-2-methylpropanamido)-3-phenylpropanoic Acid

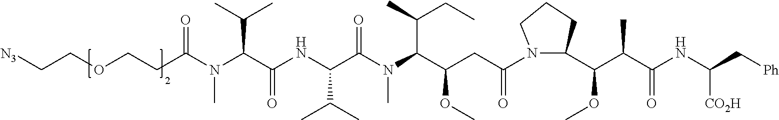

To the crude (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-pyrrolidin-2-yl)-3-methoxy-2-methylpropanamido)-3-phenylpropanoic acid (22 mg, 0.030 mmol) in a mixture of DMA (0.8 ml) and NaH₂PO₄ buffer solution (pH 7.5, 1.0 M, 0.7 ml) was added 2,5-dioxopyrrolidin-1-yl 3-(2-(2-azidoethoxy)ethoxy)propanoate (18.0 mg, 0.060 mmol) in four portions in 2 h. The mixture was stirred overnight, concentrated and purified on SiO₂ column chromatography (CH₃OH/CH₂Cl₂/HOAc 1:8:0.01) to afford the title compound (22.5 mg, 82% yield). LC-MS (ESI) m/z calcd. for C₄₆H₇₇N₈O₁₁ [M+H]⁺: 917.56, found: 917.60.

### Example 107. Synthesis of (2S)-2-((2R,3R)-3-((2S)-1-((11S,14S,17S)-1-amino-17-((R)-sec-butyl)-11,14-diisopropyl-18-methoxy-10,16-dimethyl-9,12,15-trioxo-3,6-dioxa-10,13,16-triazaicosan-20-oyl)pyrrolidin-2-yl)-3-methoxy-2-methylpropanamido)-3-phenylpropanoic Acid

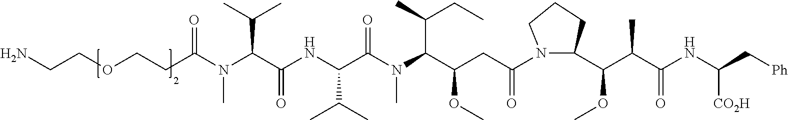

To (2S)-2-((2R,3R)-3-((2S)-1-((11S,14S,17S)-1-azido-17-((R)-sec-butyl)-11,14-diisopropyl-18-methoxy-10,16-dimethyl-9,12,15-trioxo-3,6-dioxa-10,13,16-triazaicosan-20-oyl)pyrrolidin-2-yl)-3-methoxy-2-methylpropanamido)-3-phenylpropanoic acid (22.0 mg, 0.024 mmol) in methanol (5 ml) in a hydrogenation bottle was added Pd/C (5 mg, 10% Pd, 50% wet). After air was vacuumed out and 25 psi H₂ was conducted in, the mixture was shaken for 4 h, filtered through Celite. The filtrate was concentrated to afford the crude title product (˜20 mg, 92% yield), which was used in the next step without further purification. ESI MS m/z+C₄₆H₇₉N₆O₁₁ (M+H), cacld. 891.57, found 891.60.

### Example 108. Synthesis of (S)-2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapenta-decan-15-oyl)pyrrolidin-2-yl)-3-methoxy-2-methylpropanamido)-3-phenylpropanoic Acid

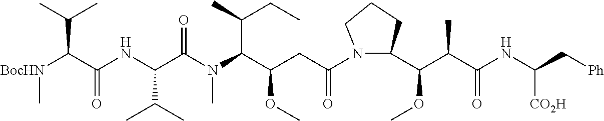

To a solution of (S)-methyl 2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapentadecan-15-oyl)pyrrolidin-2-yl)-3-methoxy-2-methylpropanamido)-3-phenylpropanoate (30 mg, 0.035 mmol) in THE (1.0 ml) was added LiOH in water (1.0M, 0.8 ml). The mixture was stirred at r.t. for 35 min, neutralized with 0.5 M H₃PO₄ to pH 6, concentrated and purified on SiO₂ column chromatography (CH₃OH/CH₂Cl₂/HOAc 1:10:0.01) to afford the title compound (25.0 mg, 85% yield). LC-MS (ESI) m/z calcd. for C₄₄H₇₄N₅O₁₀ [M+H]⁺: 832.54, found: 832.60.

### Example 109. Synthesis of (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)-pyrrolidin-2-yl)-3-methoxy-2-methylpropanamido)-3-phenylpropanoic Acid

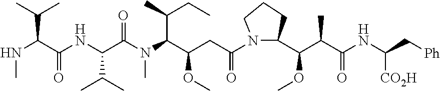 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

To a solution of (S)-2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapenta-decan-15-oyl)pyrrolidin-2-yl)-3-methoxy-2-methylpropanamido)-3-phenylpropanoic acid (25 mg, 0.030 mmol) in dioxane (2.0 ml) was added HCl (12.0M, 0.6 ml). The mixture was stirred at r.t. for 30 min, diluted with dioxane (4 ml) and toluene (4 ml), concentrated and purified on C-18 HPLC column chromatography eluted with MeOH and water (L200 mm×ϕ20 mm, v=9 ml/min, from 5% methanol to 40% methanol in 40 min) to afford the title compound (20.0 mg, 90% yield). LC-MS (ESI) m/z calcd. for C₃₉H₆₆N₅O₈ [M+H]⁺: 732.48, found: 732.90.

### Example 110. Synthesis of (S)-methyl 2-((2R,3R)-3-((S)-1-((5S,8S,11S,14S,15R)-14-((S)-sec-butyl)-8,11-diisopropyl-15-methoxy-5,7,13-trimethyl-3,6,9,12-tetraoxo-1-phenyl-2-oxa-4,7,10,13-tetraazaheptadecan-17-oyl)pyrrolidin-2-yl)-3-methoxy-2-methylpropanamido)-3-phenvlpropanoate

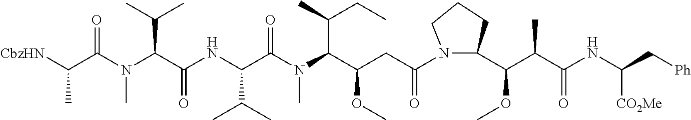 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)C(=O)[C@H](C)NC(=O)OCc1ccccc1)C(C)C]

To a solution of MMAF-OMe (0.132 g, 0.178 mmol, 1.0 eq.) and Z-L-Alanine (0.119 g, 0.533 mmol, 3.0 eq.) in anhydrous DCM (10 mL) at 0° C. was added HATU (0.135 g, 0.356 mmol, 2.0 eq.) and NMM (0.12 mL, 1.07 mmol, 6.0 eq.) in sequence. The reaction was stirred at 0° C. for 10 minutes, then warmed to room temperature and stirred overnight. The mixture was diluted with DCM and washed with water and brine, dried over anhydrous Na₂SO₄, concentrated and purified by SiO₂ column chromatography (20:1 DCM/MeOH) to give the title compound as a white foamy solid (0.148 g, 88% yield). ESI MS m/z: calcd for C₅₁H₇₉N₆O₁₁ [M+H]⁺ 951.6, found 951.6.

### Example 111. Synthesis of (S)-methyl 2-((2R,3R)-3-((S)-1-((3R,4S,5S)-4-((S)-2-((S)-2-((S)-2-amino-N-methylpropanamido)-3-methylbutanamido)-N,3-dimethylbutanamido)-3-methoxy-5-methylheptanoyl)pyrrolidin-2-yl)-3-methoxy-2-methylpropanamido)-3-phenylpropanoate

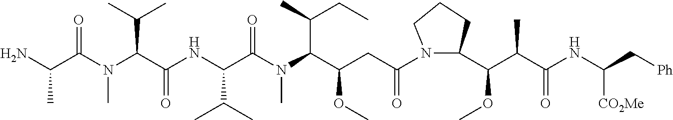 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)C(=O)[C@H](C)N)C(C)C]

To a solution of (S)-methyl 2-((2R,3R)-3-((S)-1-((5S,8S,11S,14S,15R)-14-((S)-sec-butyl)-8,11-diisopropyl-15-methoxy-5,7,13-trimethyl-3,6,9,12-tetraoxo-1-phenyl-2-oxa-4,7,10,13-tetraazaheptadecan-17-oyl)pyrrolidin-2-yl)-3-methoxy-2-methylpropanamido)-3-phenylpropanoate (0.148 g, 0.156 mmol, 1.0 equiv) in MeOH (5 mL) was added Pd/C (0.100 g, 10% Pd/C, 50% wet) in a hydrogenation bottle. The mixture was shaken for 5 h then filtered through a Celite pad. The filtrate was concentrated to give the title compound as a white foamy solid (0.122 g, 96% yield). ESI MS m/z: calcd for C₄₃H₇₃N₆O₉ [M+H]⁺ 817.5, found 817.5.

### Example 112. Synthesis of (2S)-methyl 2-((2R,3R)-3-((2S)-1-((46S,49S,52S,55S,56R)-55-((S)-sec-butyl)-37,38-bis(2-(2,5-dioxo-2,5-dihydro-1H-pyrrol-1-yl)acetamido)-1-hydroxy-49,52-diisopropyl-56-methoxy-46,48,54-trimethyl-31,36,39,44,47,50,53-heptaoxo-3,6,9,12,15,18,21,24,27-nonaoxa-30,35,40,45,48,51,54-heptaazaoctapentacontan-58-oyl)pyrrolidin-2-yl)-3-methoxy-2-methylpropanamido)-3-phenylpropanoate (A-02)

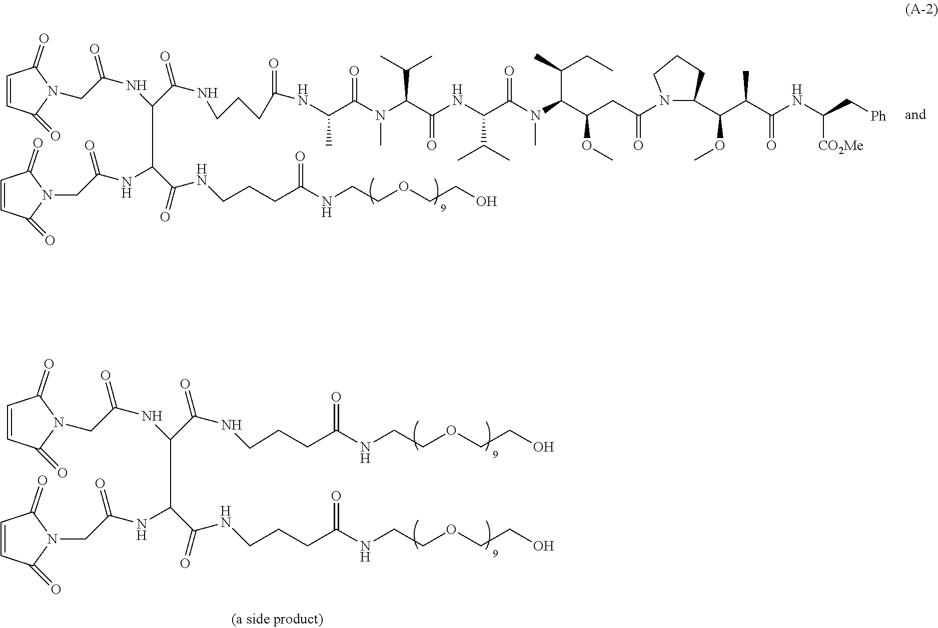

To a solution of (S)-methyl 2-((2R,3R)-3-((S)-1-((3R,4S,5S)-4-((S)-2-((S)-2-((S)-2-amino-N-methylpropanamido)-3-methylbutanamido)-N,3-dimethylbutanamido)-3-methoxy-5-methyl-heptanoyl)pyrrolidin-2-yl)-3-methoxy-2-methylpropanamido)-3-phenylpropanoate (0.122 g, 0.149 mmol, 1.0 eq.) and 4,4′-((2,3-bis(2-(2,5-dioxo-2,5-dihydro-1H-pyrrol-1-yl)acetamido)-succinyl)bis(azanediyl))dibutanoic acid (0.177 g, 0.298 mmol, 4.0 eq.) in anhydrous DMA (10 mL) was added HATU (0.270 g, 0.712 mmol) and NMM (0.030 mL, 0.267 mmol). The reaction was stirred for 2 h, then 29-amino-3,6,9,12,15,18,21,24,27-nonaoxanonacosan-1-ol (0.205 mg, 0.448 mmol) was added in. The reaction mixture was continued to stir overnight. The mixture was concentrated in vacuo and purified by SiO₂ column chromatography (10:1 to 5:1, DCM/MeOH) to give the title compound (A-2) as a white foamy solid (0.128 g, 47% yield, ESI MS m/z: calcd for C₈₇H₁₄₀N₁₃O₂₉ [M+H]⁺ 1830.98, found 1830.70), and a side product, 2,3-bis(2-(2,5-dioxo-2,5-dihydro-1H-pyrrol-1-yl)acetamido)-N¹,N⁴-bis(1-hydroxy-31-oxo-3,6,9,12,15,18,21,24,27-nonaoxa-30-azatetratriacontan-34-yl)succinamide (84 mg, 38% yield, ESI MS m/z: calcd for C₆₄H₁₁₁N₈O₃₀ [M+H]⁺ 1471.73, found 1471.95).

### Example 113. Synthesis of (2S)-2-((2R,3R)-3-((2S)-1-((56S,59S,62S,63R)-62-((S)-sec-butyl)-37,38-bis(2-(2,5-dioxo-2,5-dihydro-1H-pyrrol-1-yl)acetamido)-1-hydroxy-56,59-diisopropyl-63-methoxy-55,61-dimethyl-31,36,39,44,54,57,60-heptaoxo-3,6,9,12,15,18,21,24,27,48,51-undecaoxa-30,35,40,45,55,58,61-heptaazapentahexacontan-65-oyl)pyrrolidin-2-yl)-3-methoxy-2-methylpropanamido)-3-phenylpropanoic Acid (A-3)

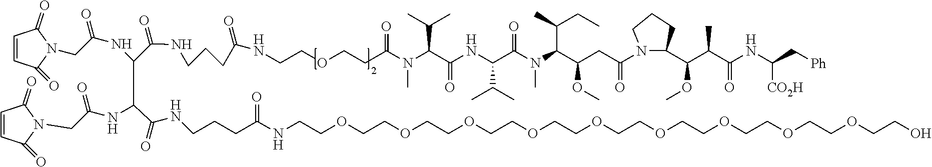

To a solution of (2S)-2-((2R,3R)-3-((2S)-1-((11S,14S,17S)-1-amino-17-((R)-sec-butyl)-11,14-diisopropyl-18-methoxy-10,16-dimethyl-9,12,15-trioxo-3,6-dioxa-10,13,16-triazaicosan-20-oyl)pyrrolidin-2-yl)-3-methoxy-2-methylpropanamido)-3-phenylpropanoic acid (0.155 g, 0.174 mmol, 1.0 eq.) in a mixture solution of DMA (10 ml) and PBS buffer (10 ml, 0.1 M NaH₂PO₄, pH 5.0) was added bis(2,5-dioxopyrrolidin-1-yl) 4,4′-((2,3-bis(2-(2,5-dioxo-2,5-dihydro-1H-pyrrol-1-yl)acetamido)succinyl)bis(azanediyl))dibutanoate (0.275 g, 0.349 mmol, 4.0 eq.). The mixture was stirred for 4 h, then then 29-amino-3,6,9,12,15,18,21,24,27-nonaoxanonacosan-1-ol (0.205 mg, 0.448 mmol) was added in. The reaction mixture was adjusted to pH 7.5 with NaHCO₃ (sat) and continued to stir overnight. The mixture was concentrated in vacuo and purified by reverse phase HPLC (250 (L) mm x 20(d) mm, C₁₈ column, 10-80% acetonitrile/water in 40 min, v=10 ml/min) to afford the title compound (142.1 mg, 43% yield, ESI MS m/z: calcd for C₉₀H₁₄₆N₁₃O₃₁ [M+H]⁺ 1905.02, found 1905.80) and a side product, 2,3-bis(2-(2,5-dioxo-2,5-dihydro-1H-pyrrol-1-yl)acetamido)-N¹,N⁴-bis(1-hydroxy-31-oxo-3,6,9,12,15,18,21,24,27-nonaoxa-30-azatetratriacontan-34-yl)succinamide (89 mg, 35% yield, ESI MS m/z: calcd for C₆₄H₁₁₁N₈O₃₀ [M+H]⁺ 1471.73, found 1471.95).

### Example 114. Synthesis of (2S,2'S)-2,2′-(((2R,2′R,3R,3′R)-3,3′-((2S,2'S)-1,1′-((3R,4S,7S,10S,47S,50S,53S,54R)-4,53-di((S)-sec-butyl)-28,29-bis(2-(2,5-dioxo-2,5-dihydro-1H-pyrrol-1-yl)acetamido)-7,10,47,50-tetraisopropyl-3,54-dimethoxy-5,11,46,52-tetramethyl-6,9,12,22,27,30,35,45,48,51-decaoxo-15,18,39,42-tetraoxa-5,8,11,21,26,31,36,46,49,52-decaazahexapentacontane-1,56-dioyl)bis(pyrrolidine-2,1-diyl))bis(3-methoxy-2-methylpropanoyl))bis(azanediyl))bis(3-phenylpropanoic Acid) (A-04)

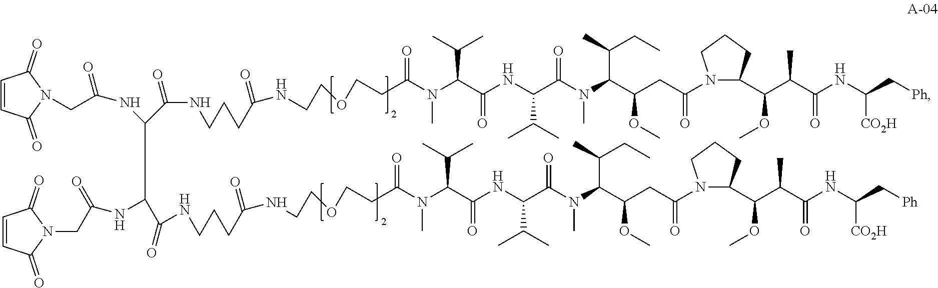

To a solution of (2S)-2-((2R,3R)-3-((2S)-1-((11S,14S,17S)-1-amino-17-((R)-sec-butyl)-11,14-diisopropyl-18-methoxy-10,16-dimethyl-9,12,15-trioxo-3,6-dioxa-10,13,16-triazaicosan-20-oyl)pyrrolidin-2-yl)-3-methoxy-2-methylpropanamido)-3-phenylpropanoic acid (0.155 g, 0.174 mmol, 1.0 eq.) in a mixture solution of DMA (10 ml) and PBS buffer (10 ml, 0.1 M NaH₂PO₄, pH 7.5) was added bis(2,5-dioxopyrrolidin-1-yl) 4,4′-((2,3-bis(2-(2,5-dioxo-2,5-dihydro-1H-pyrrol-1-yl)acetamido)succinyl)bis(azanediyl))dibutanoate (0.068 g, 0.087 mmol, 1.0 eq.). The mixture was stirred for 8 h, concentrated in vacuo and purified by reverse phase HPLC (250 (L) mm x 20(d) mm, C₁₈ column, 10-80% acetonitrile/water in 40 min, v=10 ml/min) to afford the title compound (138.1 mg, 68% yield). ESI MS m/z: calcd for C₁₁₆H₁₈₁N₁₈O₃₂ [M+H]⁺ 2338.30, found 2338.90.

### Example 115. Synthesis of (S,E)-2-methyl-N-(3-methylbutan-2-ylidene)propane-2-sulfonamide

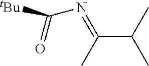

To a solution of (S)-2-methylpropane-2-sulfinamide (100 g, 0.825 mol, 1.0 eq.) in 1 L THF was added Ti(OEt)₄ (345 mL, 1.82 mol, 2.2 eq.) and 3-methyl-2-butanone (81 mL, 0.825 mol, 1.0 eq.) under N₂ at r.t. The reaction mixture was refluxed for 16 h, then cooled to r.t. and poured onto iced water. The mixture was filtered and the filter cake was washed with EtOAc. The organic layer was separated, dried over anhydrous Na₂SO₄ and concentrated to give a residue which was purified by vacuum distillation (15-20 torr, 95° C.) to afforded the title product (141 g, 90% yield) as a yellow oil. 1H NMR (500 MHz, CDCl₃) δ 2.54-2.44 (m, 1H), 2.25 (s, 3H), 1.17 (s, 9H), 1.06 (dd, J=6.9, 5.1 Hz, 6H). MS ESI m/z calcd for C₉H₁₉NaNOS [M+Na]⁺212.12; found 212.11.

### Example 116. Synthesis of (2S,3S)-2-azido-3-methylpentanoic Acid

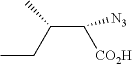

To a solution of NaN₃ (20.0 g, 308 mmol) in a mixture of water (50 mL) and dichloromethane (80 mL), cooled at 0° C., Tf₂O (10 mL, 59.2 mmol, 2.0 eq.) was added slowly. After addition, the reaction was stirred at 0° C. for 2 h, then the organic phase was separated and the aqueous phase was extracted with dichloromethane (2×40 mL). The combined organic phases were washed with saturated NaHCO₃ solution and used as is. The dichloromethane solution of triflyl azide was added to a mixture of (L)-isoleucine (4.04 g, 30.8 mmol, 1.0 eq.), K₂CO₃ (6.39 g, 46.2 mmol, 1.5 eq.), CuSO₄.5H₂O (77.4 mg, 0.31 mmol, 0.01 eq.) in water (100 ml) and methanol (200 ml). The mixture was stirred at r.t. for 16 h. The organic solvents were removed under reduced pressure and the aqueous phase was diluted with water (250 mL) and acidified to pH 6 with concentrated HCl and diluted with phosphate buffer (0.25 M, pH 6.2, 250 mL). The aqueous layer was washed with EtOAc (5×100 mL) to remove the sulfonamide by-product, and then acidified to pH 2 with concentrated HCl, extracted with EtOAc (3×150 mL). The combined organic layers were dried over anhydrous Na₂SO₄, filtered and concentrated to give the title product (4.90 g, 99% yield) as colorless oil. ¹H NMR (500 MHz, CDCl₃) δ 12.01 (s, 1H), 3.82 (d, J=5.9 Hz, 1H), 2.00 (ddd, J=10.6, 8.6, 5.5 Hz, 1H), 1.54 (dqd, J=14.8, 7.5, 4.4 Hz, 1H), 1.36-1.24 (m, 1H), 1.08-0.99 (m, 3H), 0.97-0.87 (m, 3H).

### Example 117. Synthesis of D-N-Methyl Pipecolinic Acid

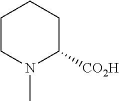

To a solution of D-pipecolinic acid (10.0 g, 77.4 mmol, 1.0 eq.) in methanol (100 mL) was added formaldehyde (37% aqueous solution, 30.8 mL, 154.8 mmol, 2.0 eq.), followed by Pd/C (10 wt %, 1.0 g). The reaction mixture was stirred under H₂ (1 atm) overnight, and then filtered through Celite, with washing of the filter pad with methanol. The filtrate was concentrated under reduced pressure to afford the title compound (10.0 g, 90% yield) as a white solid.

### Example 118. Synthesis of (R)-perfluorophenyl 1-methylpiperidine-2-carboxylate

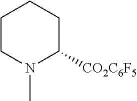

To a solution of D-N-methyl pipecolinic acid (2.65 g, 18.5 mmol) in EtOAc (50 mL) were added pentafluorophenol (3.75 g, 20.4 mmol) and DCC (4.21 g, 20.4 mmol). The reaction mixture was stirred at r.t. for 16 h, and then filtered over Celite. The filter pad was washed with 10 mL of EtOAc. The filtrate was used for the next step without further purification or concentration. MS ESI m/z calcd for C₁₃H₁₃F₅NO₂ [M+H]⁺ 309.08; found 309.60.

### Example 119. Synthesis of perfluorophenyl 2-(dimethylamino)-2-methylpropanoate

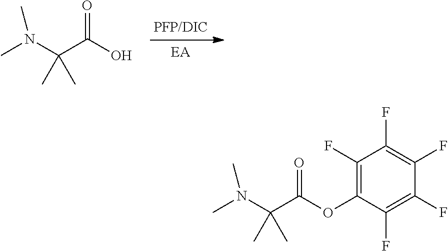

To a solution of 2-(dimethylamino)-2-methylpropanoic acid (5.00 g, 38.10 mmol) in ethyl acetate (200 ml) at 0° C. was added 2,3,4,5,6-pentafluorophenol (10.4 g, 57.0 mmol) followed by addition of DIC (8.8 mL, 57.0 mmol). The reaction mixture was warmed to RT, stirred overnight and filtered. The filtrate was concentrated to afford the title compound (12.0 g, >100% yield) which was used for the next step without further purification. MS ESI m/z calcd for C₁₂H₁₃F₅NO₂ [M+H]⁺ 298.08; found 298.60.

### Example 120. Synthesis of 2,2-diethoxyethanethioamide

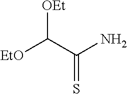

2,2-diethoxyacetonitrile (100 g, 0.774 mol, 1.0 eq.) was mixed with (NH₄)₂S aqueous solution (48%, 143 mL, 1.05 mol, 1.36 eq.) in methanol (1.5 L) at room temperature. After stirring for 16 h, the reaction mixture was concentrated and the residue was taken up in dichloromethane, washed with saturated NaHCO₃ solution and brine, dried over anhydrous Na₂SO₄ and concentrated. The residue was triturated with a solvent mixture of petroleum ether and dichloromethane. After filtration, the desired title product as a white solid was collected (100 g, 79% yield). ¹H NMR (500 MHz, CDCl₃) δ 7.81 (d, J=71.1 Hz, 2H), 5.03 (s, 1H), 3.73 (dq, J=9.4, 7.1 Hz, 2H), 3.64 (dq, J=9.4, 7.0 Hz, 2H), 1.25 (t, J=7.1 Hz, 6H).

### Example 121. Synthesis of ethyl 2-(diethoxymethyl)thiazole-4-carboxylate

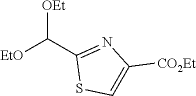 [SMILES: CCOC(=O)c1csc(C(OCC)OCC)n1]

90 g of molecular sieves (3A) was added to a mixture of 2,2-diethoxyethanethioamide (100 g, 0.61 mol, 1.0 eq.) and ethyl bromopyruvate (142 mL, 1.1 mol, 1.8 eq.) in 1 L EtOH. The mixture was refluxed (internal temperature about 60° C.) for 1h, then ethanol was removed on rotovap and the residue was taken up in dichloromethane. The solid was filtered off and the filtrate was concentrated and purified by column chromatography (PE/EtOAc 5:1-3:1) to give the title (thiazole carboxylate) compound (130 g, 82% yield) as a yellow oil.

### Example 122. Synthesis of ethyl 2-formylthiazole-4-carboxylate

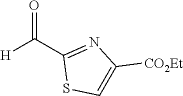 [SMILES: CCOC(=O)c1csc(C=O)n1]

To a solution of 2-(diethoxymethyl)thiazole-4-carboxylate (130 g, 0.50 mol) in acetone (1.3 L) was added 2 N HCl (85 mL, 0.165 mol, 0.33 eq.). The reaction mixture was refluxed (internal temperature about 60° C.), monitored by TLC analysis until starting material was completely consumed (about 1-2 h). Acetone was removed under reduced pressure and the residue was taken up in dichloromethane (1.3 L), washed with saturated NaHCO₃ solution, water and brine, and then dried over anhydrous Na₂SO₄. The solution was filtered and concentrated under reduced pressure. The crude product was purified by recrystallization from petreolum ether and diethyl ether to afford the title compound as a white solid (40 g, 43% yield). ¹H NMR (500 MHz, CDCl₃) δ 10.08-10.06 (m, 1H), 8.53-8.50 (m, 1H), 4.49 (q, J=7.1 Hz, 2H), 1.44 (t, J=7.1 Hz, 3H). MS ESI m/z calcd for C₇H₈NO₃S [M+H]⁺ 186.01; found 186.01.

### Example 123. Synthesis of ethyl 2-((R,E)-3-(((S)-tert-butylsulfinyl)imino)-1-hydroxy-4-methylpentyl)thiazole-4-carboxylate

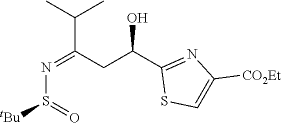 [SMILES: CCOC(=O)c1csc([C@H](O)C/C(=N\[S@@](=O)C(C)(C)C)C(C)C)n1]

To a solution of diisopropylamine (121 mL, 0.86 mol, 4.0 eq.) in dry THE (300 mL) was added n-butyllithium (2.5 M, 302 mL, 0.76 mol 3.5 eq.) at −78° C. under N₂. The reaction mixture was warmed to 0° C. over 30 min and then cooled back to −78°. (S,E)-2-methyl-N-(3-methylbutan-2-ylidene)propane-2-sulfonamide (57 g, 0.3 mol, 1.4 eq.) in THE (200 mL) was added. The reaction mixture was stirred for 1 h before ClTi(OⁱPr)₃ (168.5 g, 0.645 mol, 3.0 eq.) in THE (350 mL) was added dropwise. After stirring for 1 h, ethyl 2-formylthiazole-4-carboxylate (40 g, 0.215 mol, 1.0 eq.) dissolved in THE (175 mL) was added dropwise and the resulting reaction mixture was stirred for 2 h. The completion of the reaction was indicated by TLC analysis. The reaction was quenched by a mixture of acetic acid and THE (v/v 1:4, 200 mL), then poured onto iced water, extracted with EtOAc (4×500 mL). The organic phase was washed with water and brine, dried over anhydrous Na₂SO₄, filtered and concentrated. The residue was purified by column chromatography (DCM/EtOAc/PE 2:1:2) to afforded the title compound (60 g, 74% yield) as a colorless oil. ¹H NMR (500 MHz, CDCl₃) δ 8.13 (s, 1H), 6.63 (d, J=8.2 Hz, 1H), 5.20-5.11 (m, 1H), 4.43 (q, J=7.0 Hz, 2H), 3.42-3.28 (m, 2H), 2.89 (dt, J=13.1, 6.5 Hz, 1H), 1.42 (t, J=7.1 Hz, 3H), 1.33 (s, 9H), 1.25-1.22 (m, 6H). MS ESI m/z calcd for C₁₆H₂₆NaN₂O₄S₂ [M+Na]⁺ 397.13, found 397.11.

### Example 124. Synthesis of ethyl 2-((1R,3R)-3-((S)-1,1-dimethylethylsulfinamido)-1-hydroxy-4-methylpentyl)thiazole-4-carboxylate

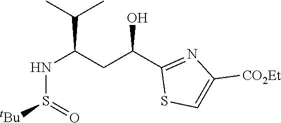 [SMILES: CCOC(=O)c1csc([C@H](O)C[C@@H](N[S@@](=O)C(C)(C)C)C(C)C)n1]

A solution of ethyl 2-((R,E)-3-(((S)-tert-butylsulfinyl)imino)-1-hydroxy-4-methylpentyl) thiazole-4-carboxylate (23.5 g, 62.7 mmol) dissolved in THE (200 mL) was cooled to −45° C. Ti(OEt)₄ (42.9 mL, 188 mmol, 3.0 eq.) was added slowly. After the completion of addition, the mixture was stirred for 1 h, before NaBH₄ (4.75 g, 126 mmol, 2.0 eq.) was added in portions. The reaction mixture was stirred at −45° C. for 3 h. TLC analysis showed some starting material still remained. The reaction was quenched with HOAc/THF (v/v 1:4, 25 mL), followed by EtOH (25 mL). The reaction mixture was poured onto ice (100 g) and warmed to r.t. After filtration over Celite, the organic phase was separated and washed with water and brine, dried over anhydrous Na₂SO₄, filtered, and concentrated. The residue was purified by column chromatography (EtOAc/PE 1:1) to deliver the title product (16.7 g, 71% yield) as a white solid. ¹H NMR (500 MHz, CDCl₃) δ 8.10 (s, 1H), 5.51 (d, J=5.8 Hz, 1H), 5.23-5.15 (m, 1H), 4.41 (q, J=7.0 Hz, 2H), 3.48-3.40 (m, 1H), 3.37 (d, J=8.3 Hz, 1H), 2.29 (t, J=13.0 Hz, 1H), 1.95-1.87 (m, 1H), 1.73-1.67 (m, 1H), 1.40 (t, J=7.1 Hz, 3H), 1.29 (s, 9H), 0.93 (d, J=7.3 Hz, 3H), 0.90 (d, J=7.2 Hz, 3H). MS ESI m/z calcd for C₁₆H₂₈NaN₂O₄S₂ [M+Na]⁺ 399.15, found 399.14.

### Example 125. Synthesis of ethyl 2-((1R,3R)-3-amino-1-hydroxy-4-methylpentyl)thiazole-4-carboxylate hydrochloride

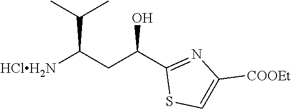

To a solution of ethyl 2-((1R,3R)-3-((S)-1,1-dimethylethylsulfinamido)-1-hydroxy-4-methylpentyl)thiazole-4-carboxylate (6.00 g, 16.0 mmol, 1.0 eq.) in ethanol (40 mL) was added 4 N HCl in dioxane (40 mL) slowly at 0° C. The reaction was allowed to warm to r.t. and stirred for 2.5 h then concentrated and triturated with petreolum ether. A white solid title compound (4.54 g, 92% yield) was collected and used in the next step.

### Example 126. Synthesis of ethyl 2-((1R,3R)-3-((2S,3S)-2-azido-3-methylpentanamido)-1-hydroxy-4-methylpentyl)thiazole-4-carboxylate

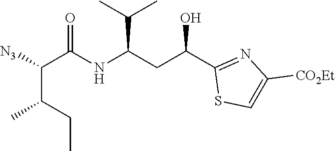 [SMILES: CCOC(=O)c1csc([C@H](O)C[C@@H](NC(=O)[C@@H](N=[N+]=[N-])[C@@H](C)CC)C(C)C)n1]

(2S,3S)-2-azido-3-methylpentanoic (5.03 g, 28.8 mmol, 2.0 eq.) was dissolved in THE (120 mL) and cooled to 0° C., to which NMM (6.2 mL, 56.0 mmol, 4.0 eq.) and isobutylchloroformate (3.7 mL, 28.8 mmol, 2.0 eq.) were added in sequence. The reaction was stirred at 0° C. for 30 min and r.t. 1.0 h, and then cooled back to 0° C. Ethyl 2-((1R,3R)-3-amino-1-hydroxy-4-methylpentyl)thiazole-4-carboxylate hydrochloride (4.54 g, 14.7 mmol, 1.0 eq.) was added in portions. After stirring at 0° C. for 30 min, the reaction was warmed to r.t. and stirred for 2 h. Water was added at 0° C. to quenched the reaction and the resulting mixture was extracted with ethyl acetate for three times. The combined organic layers were washed with 1N HCl, saturated NaHCO₃ and brine, dried over anhydrous Na₂SO₄, filtered and concentrated. The residue was purified by column chromatography (0-30% EtOAc/PE) to give a white solid title compound (4.55 g, 74% yield).

### Example 127. Synthesis of ethyl 2-((1R,3R)-3-((2S,3S)-2-azido-3-methylpentanamido)-4-methyl-1-((triethylsilyl)oxy)pentyl)thiazole-4-carboxylate

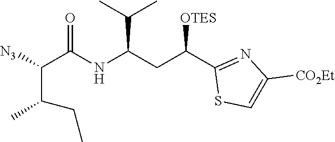

To a solution of ethyl 2-((1R,3R)-3-((2S,3S)-2-azido-3-methylpentanamido)-1-hydroxy-4-methylpentyl)thiazole-4-carboxylate (5.30 g, 12.8 mmol, 1.0 eq.) in CH₂Cl₂ (50 mL) was added imidazole (1.75 g, 25.6 mmol, 2.0 eq.), followed by chlorotriethylsilane (4.3 mL, 25.6 mmol, 2.0 eq.) at 0° C. The reaction mixture was allowed to warm to r.t. over 1 hour and stirred for an additional hour. Brine was added to the reaction mixture, the organic layer was separated and the aqueous layer was extracted with EtOAc. The combined organic phases were dried, filtered, concentrated under reduced pressure, and purified by column chromatography with a gradient of 15-35% EtOAc in petreolum ether to afford the title product (6.70 g, 99% yield) as a white solid. ¹H NMR (500 MHz, CDCl₃) δ 8.12 (s, 1H), 6.75 (d, J=8.0 Hz, 1H), 5.20-5.12 (m, 1H), 4.44 (q, J=7.0 Hz, 2H), 4.06-3.97 (m, 1H), 3.87 (d, J=3.8 Hz, 1H), 2.14 (d, J=3.8 Hz, 1H), 2.01-1.91 (m, 3H), 1.42 (t, J=7.1 Hz, 3H), 1.34-1.25 (m, 2H), 1.06 (d, J=6.8 Hz, 3H), 1.00-0.93 (m, 18H), 0.88 (dd, J=19.1, 6.8 Hz, 6H). MS ESI m/z calcd for C₂₄H₄₄N₅O₄SSi [M+H]⁺ 526.28, found 526.28.

### Example 128. Synthesis of ethyl 2-((1R,3R)-3-((2S,3S)-2-azido-N,3-dimethyl pentanamido)-4-methyl-1-((triethylsilyl)oxy)pentyl)thiazole-4-carboxylate

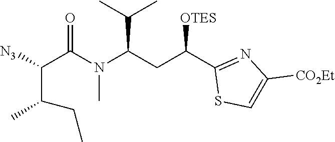

A solution of ethyl 2-((1R,3R)-3-((2S,3S)-2-azido-3-methylpentanamido)-4-methyl-1-((triethylsilyl)oxy)pentyl)thiazole-4-carboxylate (5.20 g, 9.9 mmol, 1.0 eq.) in THE (50 mL) was cooled to −45° C. and KHMDS (1M in toluene, 23.8 mL, 23.8 mmol, 2.4 eq.) was added. The resulting mixture was stirred at −45° C. for 20 min, followed by addition of methyl iodide (1.85 mL, 29.7 mmol, 3.0 eq.). The reaction mixture was warmed to r.t. over 4.5 h, then the reaction was quenched with EtOH (10 mL). The crude product was diluted with EtOAc (250 mL) and washed with brine (100 mL). The aqueous layer was extracted with EtOAc (3×50 ml). The organic layers were dried, filtered, concentrated and purified on column chromatography with a gradient of 15-35% EtOAc in petreolum ether to afford the title product (3.33 g, 63% yield) as a light yellow oil. ¹H NMR (500 MHz, CDCl₃) δ 8.09 (s, 1H), 4.95 (d, J=6.6 Hz, 1H), 4.41 (q, J=7.1 Hz, 2H), 3.56 (d, J=9.5 Hz, 1H), 2.98 (s, 3H), 2.27-2.06 (m, 4H), 1.83-1.70 (m, 2H), 1.41 (t, J=7.2 Hz, 3H), 1.29 (ddd, J=8.9, 6.8, 1.6 Hz, 3H), 1.01 (d, J=6.6 Hz, 3H), 0.96 (dt, J=8.0, 2.9 Hz, 15H), 0.92 (d, J=6.6 Hz, 3H), 0.90 (d, J=6.7 Hz, 3H). MS ESI m/z calcd for C₂₅H₄₆N₅O₄SSi [M+H]⁺ 540.30, found 540.30.

### Example 129. Synthesis of ethyl 2-((3S,6R,8R)-3-((S)-sec-butyl)-10,10-diethyl-6-isopropyl-5-methyl-1-((R)-1-methylpiperidin-2-yl)-1,4-dioxo-9-oxa-2,5-diaza-10-siladodecan-8-yl)thiazole-4-carboxylate

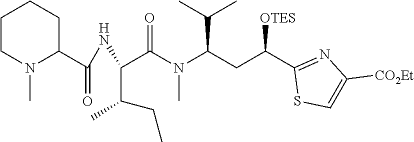

Dry Pd/C (10 wt %, 300 mg) and ethyl 2-((1R,3R)-3-((2S,3S)-2-azido-N,3-dimethyl pentanamido)-4-methyl-1-((triethylsilyl)oxy)pentyl)thiazole-4-carboxylate (3.33 g, 6.61 mmol) were added to (R)-perfluorophenyl 1-methylpiperidine-2-carboxylate in EtOAc. The reaction mixture was stirred under hydrogen atmosphere for 27 h, and then filtered through a plug of Celite, with washing of the filter pad with EtOAc. The combined organic portions were concentrated and purified by column chromatography with a gradient of 0-5% methanol in EtOAc to deliver the title product (3.90 g, 86% yield). MS ESI m/z calcd for C₃₂H₅₉N₄O₅SSi [M+H]⁺ 639.39, found 639.39.

### Example 130. Synthesis of ethyl 2-((1R,3R)-3-((2S,3S)—N,3-dimethyl-2-((R)-1-methyl piperidine-2-carboxamido)pentanamido)-1-hydroxy-4-methylpentyl)thiazole-4-carboxylate

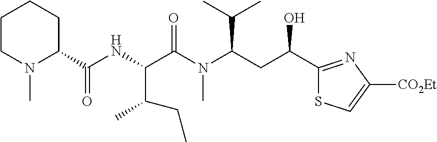 [SMILES: CCOC(=O)c1csc([C@H](O)C[C@H](C(C)C)N(C)C(=O)[C@@H](NC(=O)[C@H]2CCCCN2C)[C@@H](C)CC)n1]

Ethyl 2-((3S,6R,8R)-3-((S)-sec-butyl)-10,10-diethyl-6-isopropyl-5-methyl-1-((R)-1-methylpiperidin-2-yl)-1,4-dioxo-9-oxa-2,5-diaza-10-siladodecan-8-yl)thiazole-4-carboxylate (3.90 g, 6.1 mmol) was dissolved in deoxygenated AcOH/water/THF (v/v/v 3:1:1, 100 mL), and stirred at r.t. for 48 h. The reaction was then concentrated and purified on SiO₂ column chromatography (2:98 to 15:85 MeOH/EtOAc) to afford the title compound (2.50 g, 72% yield over 2 steps). MS ESI m/z calcd for C₂₆H₄₅N₄O₅S [M+H]⁺ 525.30, found 525.33.

### Example 131. Synthesis of 2-((1R,3R)-3-((2S,3S)—N,3-dimethyl-2-((R)-1-methylpiperidine-2-carboxamido)pentanamido)-1-hydroxy-4-methylpentyl)thiazole-4-carboxylic Acid

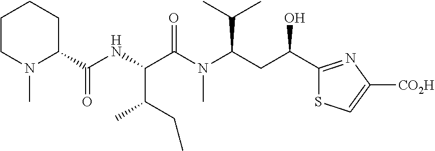 [SMILES: CC[C@H](C)[C@H](NC(=O)[C@H]1CCCCN1C)C(=O)N(C)[C@H](C[C@@H](O)c1nc(C(=O)O)cs1)C(C)C]

An aqueous solution of LiOH (0.4 N, 47.7 mL, 19.1 mmol, 4.0 eq.) was added to a solution of ethyl 2-((1R,3R)-3-((2S,3S)—N,3-dimethyl-2-((R)-1-methyl piperidine-2-carboxamido)-pentanamido)-1-hydroxy-4-methylpentyl)thiazole-4-carboxylate (2.50 g, 4.76 mmol, 1.0 eq.) in dioxane (47.7 mL) at 0° C. The reaction mixture was stirred at r.t. for 2 h and then concentrated. SiO₂ column chromatographic purification (100% CH₂Cl₂ then CH₂Cl₂/MeOH/NH₄OH 80:20:1) afforded the title compound (2.36 g, 99% yield) as an amorphous solid. MS ESI m/z calcd for C₂₄H₄₁N₄O₅S [M+H]⁺ 497.27, found 497.28.

### Example 132. Synthesis of 2-((1R,3R)-1-acetoxy-3-((2S,3S)—N,3-dimethyl-2-((R)-1-methylpiperidine-2-carboxamido)pentanamido)-4-methylpentyl)thiazole-4-carboxylic Acid

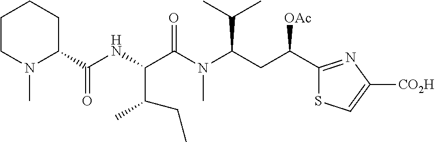 [SMILES: CC[C@H](C)[C@H](NC(=O)[C@H]1CCCCN1C)C(=O)N(C)[C@H](C[C@@H](OC(C)=O)c1nc(C(=O)O)cs1)C(C)C]

To a solution of 2-((1R,3R)-3-((2S,3S)—N,3-dimethyl-2-((R)-1-methylpiperidine-2-carboxamido)pentanamido)-1-hydroxy-4-methylpentyl)thiazole-4-carboxylic acid (2.36 g, 4.75 mmol) in pyridine (50 mL) at 0° C., acetic anhydride (2.25 mL, 24 mmol) was added slowly. The reaction mixture was warmed to r.t. over 2 h and stirred at r.t. for 24 h. The reaction was concentrated and the residue was purified on reverse phase HPLC (C₁₈ column, 50 mm (d)×250 (mm), 50 ml/min, 10-90% acetonitrile/water in 45 min) to afford the title compound (2.25 g, 88% yield) as an amorphous white solid. MS ESI m/z calcd for C₂₆H₄₃N₄O₆S [M+H]⁺ 539.28, found 539.28.

### Example 133. Synthesis of (1R,3R)-3-((2S,3S)—N,3-dimethyl-2-((R)-1-methylpiperidine-2-carboxamido)pentanamido)-4-methyl-1-(4-(perfluorobenzoyl)thiazol-2-yl)pentyl Acetate

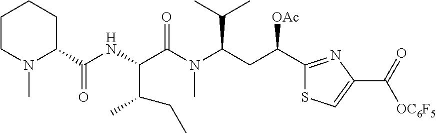

To a solution of 2-((1R,3R)-1-acetoxy-3-((2S,3S)—N,3-dimethyl-2-((R)-1-methyl-piperidine-2-carboxamido)pentanamido)-4-methylpentyl)thiazole-4-carboxylic acid (860 mg, 1.60 mmol, 1.0 eq.) in dichloromethane (20 mL) was added pentafluorophenol (440 mg, 2.40 mmol, 1.5 eq.) and N,N′-diisopropylcarbodiimide (220 mg, 1.75 mmol, 1.1 eq.) at 0° C. The reaction mixture was warmed to room temperature and stirred overnight. After the solvent was removed under reduced pressure, the reaction mixture was diluted with EtOAc (20 mL) then filtered over Celite. The filtrate was concentrated and purified on SiO₂ column chromatography (1:10 to 1:3 EtOAc/DCM) to afford the title compound (935.3 mg, 82% yield), which was used directly for the next step. MS ESI m/z calcd for C₃₂H₄₂F₅N₄O₆S [M+H]⁺ 704.28, found 704.60.

### Example 134. Synthesis of ethyl 2-((6S,9R,11R)-6-((S)-sec-butyl)-13,13-diethyl-9-isopropyl-2,3,3,8-tetramethyl-4,7-dioxo-12-oxa-2,5,8-triaza-13-silapentadecan-11-yl)thiazole-4-carboxylate

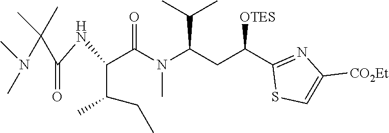

Dry Pd/C (10 wt %, 300 mg) and ethyl 2-((1R,3R)-3-((2S,3S)-2-azido-N,3-dimethyl pentanamido)-4-methyl-1-((triethylsilyl)oxy)pentyl)thiazole-4-carboxylate (3.33 g, 6.16 mmol) were added to perfluorophenyl 2-(dimethylamino)-2-methylpropanoate (˜2.75 g, 1.5 eq crude) in EtOAc. The reaction mixture was stirred under hydrogen atmosphere for 27 h, and then filtered through a plug of Celite, with washing of the filter pad with EtOAc. The combined organic portions were concentrated and purified by column chromatography with a gradient of 0-5% methanol in EtOAc to deliver the title product (3.24 g, 84% yield). MS ESI m/z calcd for C₃₁H₅₉N₄O₅SSi [M+H]⁺ 626.39, found 626.95.

### Example 135. Synthesis of ethyl 2-((1R,3R)-3-((2S,3S)-2-(2-(dimethylamino)-2-methylpropanamido)-N,3-dimethylpentanamido)-1-hydroxy-4-methylpentyl)thiazole-4-carboxylate

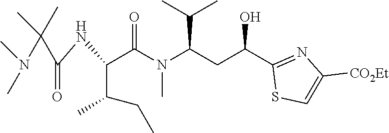 [SMILES: CCOC(=O)c1csc([C@H](O)C[C@H](C(C)C)N(C)C(=O)[C@@H](NC(=O)C(C)(C)N(C)C)[C@@H](C)CC)n1]

Ethyl 2-((6S,9R,11R)-6-((S)-sec-butyl)-13,13-diethyl-9-isopropyl-2,3,3,8-tetramethyl-4,7-dioxo-12-oxa-2,5,8-triaza-13-silapentadecan-11-yl)thiazole-4-carboxylate (3.20 g, 5.11 mmol) was dissolved in deoxygenated AcOH/water/THF (v/v/v 3:1:1, 100 mL), and stirred at r.t. for 48 h. The reaction was then concentrated and purified on SiO₂ column chromatography (2:98 to 15:85 MeOH/EtOAc) to afford the title compound (2.33 g, 89% yield). MS ESI m/z calcd for C₂₅H₄₅N₄O₅S [M+H]⁺ 512.30, found 512.45.

### Example 136. Synthesis of 2-((1R,3R)-3-((2S,3S)-2-(2-(dimethylamino)-2-methylpropanamido)-N,3-dimethylpentanamido)-1-hydroxy-4-methylpentyl)thiazole-4-carboxylic Acid

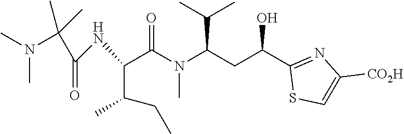 [SMILES: CC[C@H](C)[C@H](NC(=O)C(C)(C)N(C)C)C(=O)N(C)[C@H](C[C@@H](O)c1nc(C(=O)O)cs1)C(C)C]

An aqueous solution of LiOH (0.4 N, 47.7 mL, 19.1 mmol, 4.0 eq.) was added to a solution of ethyl 2-((1R,3R)-3-((2S,3S)-2-(2-(dimethylamino)-2-methylpropanamido)-N,3-dimethylpentanamido)-1-hydroxy-4-methylpentyl)thiazole-4-carboxylate (2.30 g, 4.50 mmol, 1.0 eq.) in dioxane (50 mL) at 0° C. The reaction mixture was stirred at r.t. for 2 h and then concentrated. SiO₂ column chromatographic purification (100% CH₂Cl₂ then CH₂Cl₂/MeOH/NH₄OH 80:20:1) afforded the title compound (2.13 g, 98% yield) as an amorphous solid. MS ESI m/z calcd for C₂₃H₄₁N₄O₅S [M+H]⁺ 485.27, found 485.55.

### Example 137. Synthesis of 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylic Acid

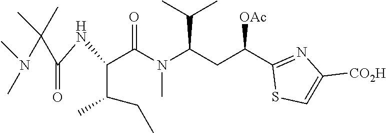 [SMILES: CC[C@H](C)[C@H](NC(=O)C(C)(C)N(C)C)C(=O)N(C)[C@H](C[C@@H](OC(C)=O)c1nc(C(=O)O)cs1)C(C)C]

To a solution of 2-((1R,3R)-3-((2S,3S)-2-(2-(dimethylamino)-2-methylpropanamido)-N,3-dimethylpentanamido)-1-hydroxy-4-methylpentyl)thiazole-4-carboxylic acid (2.10 g, 4.33 mmol) in pyridine (50 mL) at 0° C., acetic anhydride (2.25 mL, 24 mmol) was added slowly. The reaction mixture was warmed to r.t. over 2 h and stirred at r.t. for 24 h. The reaction was concentrated and the residue was purified on reverse phase HPLC (C₁s column, 50 mm (d)×250 (mm), 50 ml/min, 10-90% acetonitrile/water in 45 min) to afford the title compound (1.95 g, 86% yield) as an amorphous white solid. MS ESI m/z calcd for C₂₅H₄₃N₄O₆S [M+H]⁺ 526.28, found 526.80.

### Example 138. Synthesis of perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate

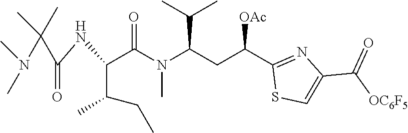

To a solution of2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylic acid (1.90 g, 3.61 mmol, 1.0 eq.) in dichloromethane (70 mL) was added pentafluorophenol (1.00 g, 5.43 mmol, 1.5 eq.) and N,N′-diisopropylcarbodiimide (512 mg, 3.96 mmol, 1.1 eq.) at 0° C. The reaction mixture was warmed to room temperature and stirred overnight. After the solvent was removed under reduced pressure, the reaction mixture was diluted with EtOAc (80 mL) then filtered over Celite. The filtrate was concentrated and purified on SiO₂ column chromatography (1:10 to 1:3 EtOAc/DCM) to afford the title compound (2.09 g, 84% yield), which was used directly for the next step. MS ESI m/z calcd for C₃₁H₄₂F₅N₄O₆S [M+H]⁺ 693.27, found 693.60.

### Example 139. Synthesis of tert-butyl 2-(triphenylphosphoranylidene)propanoate

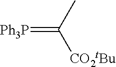

A mixture of tert-butyl-2-bromopropanoate (15.5 g, 74.1 mmol, 1.0 eq.) and triphenyl phosphine (19.4 g, 74.1 mmol, 1.0 eq.) in dry acetonitrile (45 mL) was stirred at room temperature for 18 h. Acetonitrile was removed under reduced pressure and toluene was added to crash out a white precipitate. Toluene was then decanted off and the white solid was dissolved in dichloromethane (100 mL) and transferred to a separatory funnel. 10% NaOH (100 mL) was added to the funnel, and the organic layer immediately turned yellow after shaking. The organic layer was separated and the aqueous layer was extracted with dichloromethane (30 mL) once. The dichloromethane layers were combined and washed with brine (50 mL) once, then dried over Na₂SO₄, filtered and concentrated, giving the ylide as a yellow solid (16.8 g, 58%).

### Example 140. Synthesis of (S)-methyl 3-(4-(benzyloxy)phenyl)-2-((tert-butoxy carbonyl)amino)propanoate

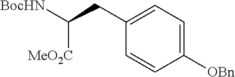 [SMILES: COC(=O)[C@H](Cc1ccc(OCc2ccccc2)cc1)NC(=O)OC(C)(C)C]

To a mixture of Boc-L-Tyr-OMe (20.0 g, 67.7 mmol, 1.0 eq.), K₂CO₃ (14.0 g, 101.6 mmol, 1.5 eq.) and KI (1.12 g, 6.77 mmol, 0.1 eq.) in acetone (100 mL) was added BnBr (10.5 mL, 81.3 mmol, 1.2 eq.) slowly. The mixture was then refluxed overnight. Water (250 mL) was added and the reaction mixture was extracted with EtOAc (3×100 mL). The combined organic layers were washed with brine (300 mL), dried over anhydrous Na₂SO₄, filtered, concentrated and purified by SiO₂ column chromatography (4:1 hexanes/EtOAc) to give a white solid title compound (26.12 g, 99% yield). ¹H NMR (500 MHz, CDCl₃) δ 7.44-7.41 (m, 2H), 7.41-7.36 (m, 2H), 7.35-7.30 (m, 1H), 7.04 (d, J=8.5 Hz, 2H), 6.93-6.89 (m, 2H), 5.04 (s, 2H), 4.97 (d, J=7.7 Hz, 1H), 4.55 (d, J=6.9 Hz, 1H), 3.71 (s, 3H), 3.03 (dd, J=14.4, 5.7 Hz, 2H), 1.44 (d, J=18.6 Hz, 10H). MS ESI m/z calcd for C₂₂H₂₇NO₅Na [M+Na]⁺ 408.18, found 408.11.

### Example 141. Synthesis of (S)-tert-butyl (1-(4-(benzyloxy)phenyl)-3-oxopropan-2-yl)carbamate

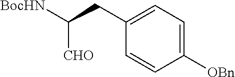 [SMILES: CC(C)(C)OC(=O)N[C@H](C=O)Cc1ccc(OCc2ccccc2)cc1]

To a solution of (S)-methyl 3-(4-(benzyloxy)phenyl)-2-((tert-butoxy carbonyl)amino)-propanoate (26.1 g, 67.8 mmol, 1.0 eq.) in anhydrous dichloromethane (450 mL) at −78° C. was added DIBAL (1.0 M in hexanes, 163 mL, 2.2 eq.) in 1 h. The mixture was stirred at −78° C. for 3 h and then quenched with 50 mL of ethanol. 1N HCl was added dropwise until pH 4 was reached. The resulting mixture was allowed to warm to 0° C. Layers were separated and the aqueous layer was further extracted with EtOAc (3×100 mL). The combined organic solution was washed with brine, dried over anhydrous Na₂SO₄, and concentrated. Trituration with PE/EtOAc and filtration gave a white solid title compound (18.3 g, 76% yield). MS ESI m/z calcd for C₂₂H₂₇NO₅Na [M+Na]⁺ 378.11, found 378.11.

### Example 142. Synthesis of (S,Z)-tert-butyl 5-(4-(benzyloxy)phenyl)-4-((tert-but oxycarbonyl)amino)-2-methylpent-2-enoate

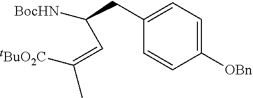 [SMILES: C/C(=C/[C@H](Cc1ccc(OCc2ccccc2)cc1)NC(=O)OC(C)(C)C)C(=O)OC(C)(C)C]

(S)-tert-Butyl (1-(4-(benzyloxy)phenyl)-3-oxopropan-2-yl)carbamate (0.84 g, 2 mmol, 1.0 eq.) was dissolved in dry dichloromethane (50 mL), to which tert-butyl 2-(triphenyl-phosphoranylidene)propanoate (1.6 g, 4 mmol, 2.0 eq.) was added and the solution was stirred at r.t. for 1.5 h as determined complete by TLC. Purification by column chromatography (10-50% EtOAc/hexanes) afforded the title compound (1.16 g, 98% yield).

### Example 143. Synthesis of (4R)-tert-butyl 4-((tert-butoxycarbonyl)amino)-5-(4-hydroxyphenyl)-2-methylpentanoate

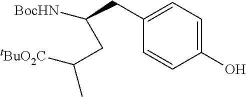 [SMILES: CC(C[C@H](Cc1ccc(O)cc1)NC(=O)OC(C)(C)C)C(=O)OC(C)(C)C]

(S,Z)-tert-Butyl 5-(4-(benzyloxy)phenyl)-4-((tert-but oxycarbonyl)amino)-2-methylpent-2-enoate (467 mg, 1 mmol) was dissolved in methanol (30 mL) and hydrogenated (1 atm) with Pd/C catalyst (10 wt %, 250 mg) at r.t. overnight. The catalyst was filtered off and the filtrate were concentrated under reduced pressure to afford the title compound (379 mg, 99% yield).

### Example 144. Synthesis of (4R)-tert-butyl 4-((tert-butoxycarbonyl)amino)-5-(4-hydroxy-3-nitrophenyl)-2-methylpentanoate

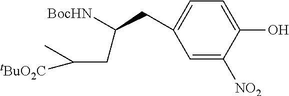 [SMILES: CC(C[C@H](Cc1ccc(O)c([N+](=O)[O-])c1)NC(=O)OC(C)(C)C)C(=O)OC(C)(C)C]

(4R)-tert-Butyl 4-((tert-butoxycarbonyl)amino)-5-(4-hydroxyphenyl)-2-methylpentanoate (379 mg, 1 mmol, 1.0 eq.) was dissolved in THE (20 mL), to which a solution of tert-butyl nitrite (315 mg, 3 mmol, 3.0 eq.) in THE (2 mL) was added. The reaction was stirred at r.t. for 3 h and then poured onto water, extracted with EtOAc (2×50 mL) and the combined organic phases were washed with brine (50 mL), dried over anhydrous Na₂SO₄, filtered and concentrated. Purification by column chromatography (10-50% EtOAc/hexanes) afforded the title compound (300 mg, 71% yield).

### Example 145. Synthesis of (4R)-tert-butyl 5-(3-amino-4-hydroxyphenyl)-4-((tert-butoxycarbonyl)amino)-2-methylpentanoate

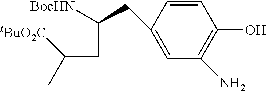 [SMILES: CC(C[C@H](Cc1ccc(O)c(N)c1)NC(=O)OC(C)(C)C)C(=O)OC(C)(C)C]

(4R)-Tert-butyl 4-((tert-butoxycarbonyl)amino)-5-(4-hydroxy-3-nitrophenyl)-2-methylpentanoate (200 mg, 0.47 mmol) was dissolved in EtOAc (30 mL) and mixed with palladium catalyst (10% on carbon, 100 mg), then hydrogenated (1 atm) at r.t. for 2 h. The catalyst was filtered off and all volatiles were removed under vacuum, which afforded the title compound (185 mg, 99%).

Alternatively, (4R)-tert-butyl 4-((tert-butoxycarbonyl)amino)-5-(4-hydroxy-3-nitrophenyl)-2-methylpentanoate (56 mg, 0.132 mmol) was dissolved in EtOAc (20 mL) and mixed with Pd/C catalyst (10 wt %, 50 mg) and hydrogenated (1 atm) at r.t. for 3 h. The catalyst was filtered off and all volatiles were removed under vacuum to afford the title compound (52 mg, 99% yield). MS ESI m/z calcd for C₂₁H₃₅N₂O₅ [M+H]⁺ 395.25, found 395.26.

### Example 146. Synthesis of (4R)-tert-butyl 4-((tert-butoxycarbonyl)amino)-5-(4-((tert-butyldimethylsilyl)oxy)-3-nitrophenyl)-2-methylpentanoate

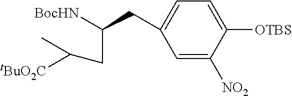 [SMILES: CC(C[C@H](Cc1ccc(O[Si](C)(C)C(C)(C)C)c([N+](=O)[O-])c1)NC(=O)OC(C)(C)C)C(=O)OC(C)(C)C]

To a solution of (4R)-tert-butyl 4-((tert-butoxycarbonyl)amino)-5-(4-hydroxy-3-nitrophenyl)-2-methylpentanoate (424 mg, 1 mmol) in DCM (20 mL), imidazole (408 mg, 6 mmol) and tert-butylchlorodimethylsilane (602 mg, 4 mmol) were added. The resulting solution was stirred at r.t. for 3 h. Afterwards, the reaction mixture was washed with brine (50 mL), dried over anhydrous Na₂SO₄, concentrated and purified by column chromatography (10% to 30% EtOAc/hexanes) to yield the title compound (344 mg, 64% yield).

### Example 147. Synthesis of (4R)-tert-butyl 5-(3-amino-4-((tert-butyldimethylsilyl) oxy)phenyl)-4-((tert-butoxycarbonyl)amino)-2-methylpentanoaten

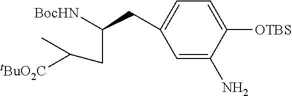 [SMILES: CC(C[C@H](Cc1ccc(O[Si](C)(C)C(C)(C)C)c(N)c1)NC(=O)OC(C)(C)C)C(=O)OC(C)(C)C]

(4R)-tert-Butyl 4-((tert-butoxycarbonyl)amino)-5-(4-((tert-butyldimethylsilyl)oxy)-3-nitrophenyl)-2-methylpentanoate (200 mg, 0.37 mmol) was dissolved in EtOAc (30 mL), mixed with palladium catalyst (10 wt % on carbon, 100 mg) and hydrogenated (1 atm) at r.t. for 2 h. The catalyst was filtered off and all volatiles were removed under vacuum to afford the title compound (187 mg, 99% yield).

### Example 148. Synthesis of 2-(1-azido-14,17-dimethyl-12,15-dioxo-3,6,9-trioxa-13,16-diazaoctadecanamido)-4-((2R)-5-(tert-butoxy)-2-((tert-butoxycarbonyl)amino)-4-methyl-5-oxopentyl)phenyl 1-azido-14,17-dimethyl-12,15-dioxo-3,6,9-trioxa-13,16-diazaoctadecan-18-oate

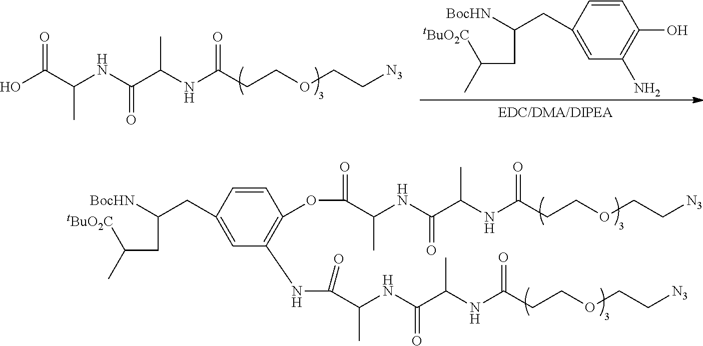

To a solution of 1-azido-14,17-dimethyl-12,15-dioxo-3,6,9-trioxa-13,16-diazaoctadecan-18-oic acid (1.50 g, 3.85 mmol) and (4R)-tert-butyl 5-(3-amino-4-hydroxyphenyl)-4-((tert-butoxycarbonyl)amino)-2-methylpentanoate (0.75 g, 1.90 mmol) in DMA (40 ml) was added EDC (2.05 g, 10.67 mmol) and DIPEA (0.70 ml, 4.0 mmol). The mixture was stirred for overnight, concentrated and purified on SiO₂ column eluted with EtOAc/CH₂Cl₂ (1:5 to 1:1) to afford the title compound (2.01 g, 82% yield, ˜95% pure by HPLC). MS ESI m/z calcd for C₅₁H₈₅N₁₂O₁₇ [M+H]⁺ 1137.61, found 1137.90.

### Example 149. Synthesis of (4R)-tert-butyl 5-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxa-heptaazacyclohexatetracontin-46-yl)-4-((tert-butoxycarbonyl)amino)-2-methylpentanoate

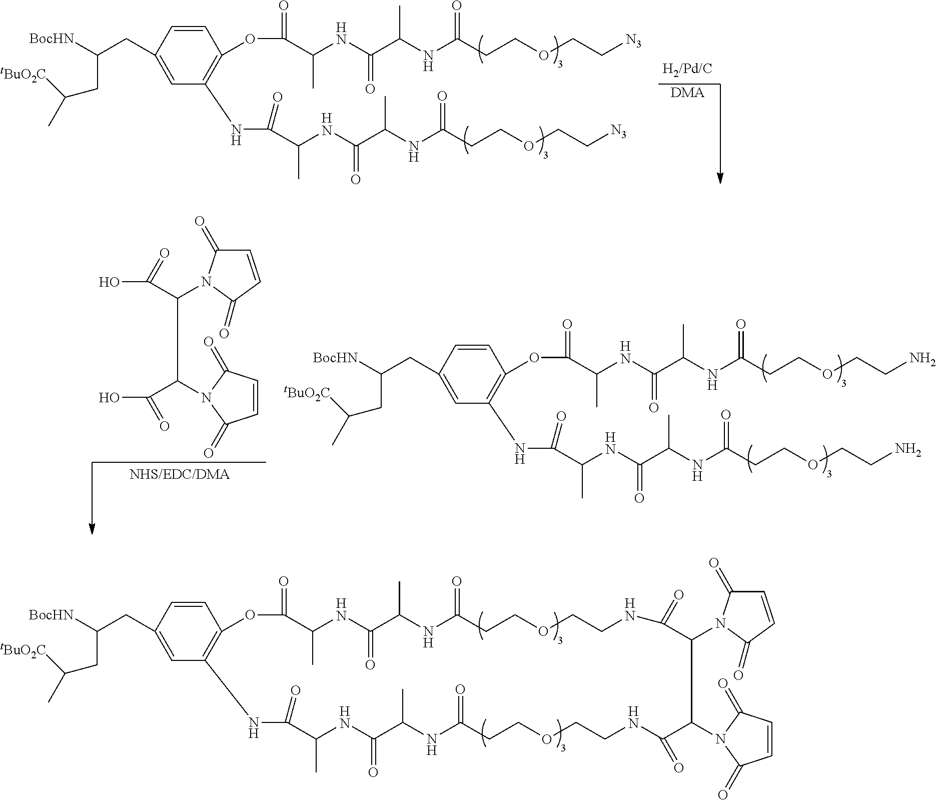

2-(1-Azido-14,17-dimethyl-12,15-dioxo-3,6,9-trioxa-13,16-diazaoctadecanamido)-4-((2R)-5-(tert-butoxy)-2-((tert-butoxycarbonyl)amino)-4-methyl-5-oxopentyl)phenyl 1-azido-14,17-dimethyl-12,15-dioxo-3,6,9-trioxa-13,16-diazaoctadecan-18-oate (900 mg, 0.79 mmol) was dissolved in EtOAc (30 mL), mixed with palladium catalyst (10 wt % on carbon, 100 mg) and hydrogenated (1 atm) at r.t. for 4 h. The catalyst was filtered off and all volatiles were removed under vacuum to afford 2-(1-amino-14,17-dimethyl-12,15-dioxo-3,6,9-trioxa-13,16-diazaoctadecanamido)-4-((2R)-5-(tert-butoxy)-2-((tert-butoxycarbonyl)amino)-4-methyl-5-oxopentyl)phenyl 1-amino-14,17-dimethyl-12,15-dioxo-3,6,9-trioxa-13,16-diazaoctadecan-18-oate (815 mg, 96% yield) which was used immediately without further purification. MS ESI m/z calcd for C₅₁H₈₈N₈O₁₇ [M+H]⁺ 1085.62, found 1085.95.

The diamino compound (810 mg, 0.75 mmol) and 2,3-bis(2,5-dioxo-2,5-dihydro-1H-pyrrol-1-yl)succinic acid (231 mg, 0.75 mmol) in DMA (10 ml) was added EDC (1.25 g, 6.51 mmol) and DIPEA (0.35 ml, 2.0 mmol). The mixture was stirred for overnight, concentrated and purified on SiO₂ column eluted with EtOAc/CH₂Cl₂ (1:5 to 1:1) to afford the title compound (844 mg, 83% yield, ˜95% pure by HPLC). MS ESI m/z calcd for C₆₃H₉₂N₁₀O₂₃ [M+H]⁺ 1357.63, found 1357.95.

### Example 150. Synthesis of (2R)-1-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaazacyclohexatetracontin-46-yl)-4-carboxypentan-2-aminium

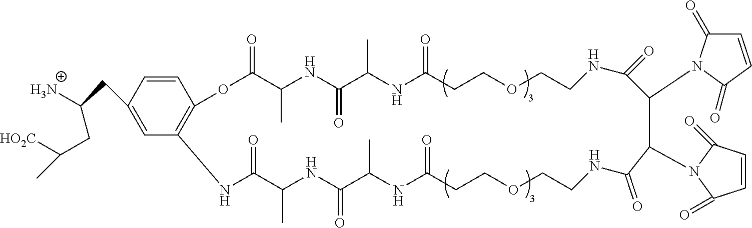

(4R)-Tert-butyl 5-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxa-heptaazacyclohexatetracontin-46-yl)-4-((tert-butoxycarbonyl)-amino)-2-methylpentanoate (840 mg, 0.62 mmol) was dissolved in the mixture of CH₂Cl₂ (6 ml) and TFA (4 ml). The mixture was stirred for overnight, diluted with toluene (10 ml), concentrated to afford the title compound (7.43 g, 100% yield, ˜91% pure by HPLC) which was used for the next step without further purification. MS ESI m/z calcd for C₅₄H₇₆N₁₀O₂₁ [M+H]⁺ 1200.51, found 1200.95.

### Example 151. Synthesis of (4R)-4-(2-((1R,3R)-1-acetoxy-3-((2S,3S)—N,3-dimethyl-2-((R)-1-methylpiperidine-2-carboxamido)pentanamido)-4-methylpentyl)thiazole-4-carboxamido)-5-(3-(3-(2-(2-azidoethoxy)ethoxy)propanamido)-4-hydroxyphenyl)-2-methylpentanoic acid

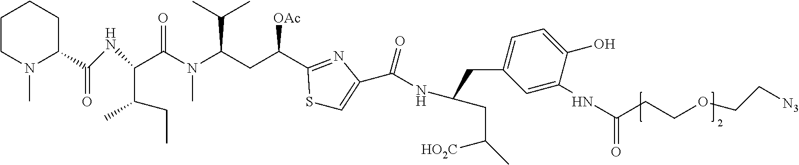

To a solution of (4R)-4-(2-((1R,3R)-1-acetoxy-3-((2S,3S)—N,3-dimethyl-2-((R) -1-methylpiperidine-2-carboxamido)pentanamido)-4-methylpentyl)thiazole-4-carboxamido)-5-(3-amino-4-hydroxyphenyl)-2-methylpentanoic acid (Huang Y. et al, Med Chem. #44, 249^{th} ACS National Meeting, Denver, Colo., Mar. 22˜26, 2015; WO2014009774) (100 mg, 0.131 mmol) in the mixture of DMA (10 ml) and NaH₂PO₄ buffer solution (pH 7.5, 1.0 M, 0.7 ml) was added 2,5-dioxopyrrolidin-1-yl 3-(2-(2-azidoethoxy)ethoxy)propanoate (80.0 mg, 0.266 mmol) in four portions in 2 h. The mixture was stirred overnight, concentrated and purified on C₁₈ preparative HPLC (3.0×25 cm, 25 ml/min), eluted with from 80% water/methanol to 10% water/methanol in 45 min to afford the title compound (101.5 mg, 82% yield). LC-MS (ESI) m/z calcd. for C₄₅H₇₀N₉O₁₁S [M+H]⁺: 944.48, found: 944.70.

### Example 152. Synthesis of (4R)-4-(2-((1R,3R)-1-acetoxy-3-((2S,3S)—N,3-dimethyl-2-((R)-1-methyl-piperidine-2-carboxamido)pentanamido)-4-methylpentyl)thiazole-4-carboxamido)-5-(3-(3-(2-(2-aminoethoxy)ethoxy)propanamido)-4-hydroxyphenyl)-2-methylpentanoic Acid

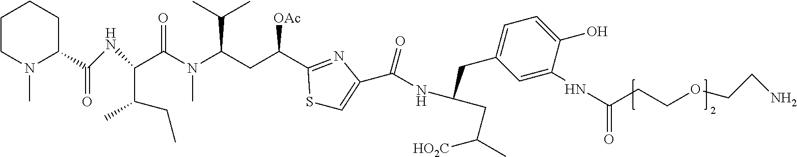

To a solution of (4R)-4-(2-((1R,3R)-1-acetoxy-3-((2S,3S)—N,3-dimethyl-2-((R)-1-methylpiperidine-2-carboxamido)pentanamido)-4-methylpentyl)thiazole-4-carboxamido)-5-(3-(3-(2-(2-azidoethoxy)ethoxy)propanamido)-4-hydroxyphenyl)-2-methylpentanoic acid (100.0 mg, 0.106 mmol) in methanol (25 ml) containing 0.1% HCl in a hydrogenation bottle was added Pd/C (25 mg, 10% Pd, 50% wet). After air was vacuumed out in the vessel and 35 psi H₂ was conducted in, the mixture was shaken for 4 h, filtered through Celite. The filtrate was concentrated and purified on C₁₈ preparative HPLC (3.0×25 cm, 25 ml/min), eluted with from 85% water/methanol to 15% water/methanol in 45 min to afford the title compound (77.5 mg, 79% yield). LC-MS (ESI) m/z calcd. for C₄₅H₇₂N₇O₁₁S [M+H]⁺: 918.49, found: 918.60.

### Example 153. Synthesis of (4R)-tert-butyl 5-(4-acetoxy-3-nitrophenyl)-4-((tert-butoxycarbonyl)amino)-2-methylpentanoate

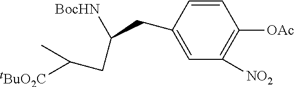 [SMILES: CC(=O)Oc1ccc(C[C@@H](CC(C)C(=O)OC(C)(C)C)NC(=O)OC(C)(C)C)cc1[N+](=O)[O-]]

To a solution of compound 190 (107.1 mg, 0.252 mmol) in dichloromethane (4.0 mL) at 0° C. was added acetic anhydride (0.11 mL, 1.17 mmol) and triethylamine (0.16 mL) in sequence. The reaction was then warmed to r.t. and stirred for 1 h, diluted with dichloromethane and washed with water and brine, dried over anhydrous Na₂SO₄, filtered and concentrated. The residue was purified by column chromatography (0-15% EA/PE) to give a colorless oil (120.3 mg, theoretical yield). MS ESI m/z calcd for C₂₃H₃₅N₂O₈ [M+H]⁺ 467.23, found 467.23.

### Example 154. Synthesis of (4R)-tert-butyl 5-(4-acetoxy-3-aminophenyl)-4-((tert-butoxycarbonyl)amino)-2-methylpentanoate

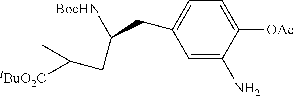 [SMILES: CC(=O)Oc1ccc(C[C@@H](CC(C)C(=O)OC(C)(C)C)NC(=O)OC(C)(C)C)cc1N]

(4R)-Tert-butyl 5-(4-acetoxy-3-nitrophenyl)-4-((tert-butoxycarbonyl)amino)-2-methylpentanoate (120.3 mg, 0.258 mmol) was dissolved in ethyl acetate (5 mL) and acetic acid (0.5 mL). To which Pd/C (10 wt %, 10 mg) was added and the mixture was stirred under H₂ balloon at r.t. for 30 min before filtration through a Celite pad with washing of the pad with ethyl acetate. The filtrate was concentrated and purified by column chromatography (0-25% EA/PE) to give a yellow oil (120.9 mg, theoretical yield). MS ESI m/z calcd for C₂₃H₃₇N₂O₆ [M+H]⁺ 437.26, found 437.28.

### Example 155. Synthesis of (4R)-ethyl 5-(3-(4-(((benzyloxy)carbonyl)amino) butanamido)-4-((tert-butyldimethylsilyl)oxy)phenyl)-4-((tert-butoxycarbonyl)amino)-2-methylpentanoate

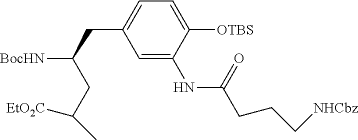 [SMILES: CCOC(=O)C(C)C[C@H](Cc1ccc(O[Si](C)(C)C(C)(C)C)c(NC(=O)CCCNC(=O)OCc2ccccc2)c1)NC(=O)OC(C)(C)C]

2,5-dioxopyrrolidin-1-yl 4-(((benzyloxy)carbonyl)amino)butanoate (0.396 g, 1.2 mmol) and (4R)-ethyl 5-(3-amino-4-hydroxyphenyl)-4-((tert-butoxycarbonyl) amino)-2-methylpentanoate (0.44 g, 1.2 mmol) were dissolved in EtOH (10 mL), and phosphate buffer solution (pH=7.5, 0.1M, 2 ml) was added. The reaction mixture was stirred at r.t. overnight and then the solvent was removed under reduced pressure and the residue purified by SiO₂ column chromatography to give the title product (0.485 g, 70%). ESI: m/z: calcd for C₃₁H₄₄N₃O₈ [M+H]⁺:586.31, found 586.31.

### Example 156. Synthesis of (4R)-ethyl 5-(3-(4-aminobutanamido)-4-((tert-butyl dimethylsilyl)oxy)phenyl)-4-((tert-butoxycarbonyl)amino)-2-methylpentanoate

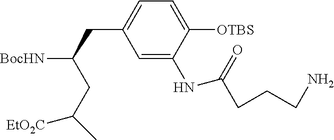 [SMILES: CCOC(=O)C(C)C[C@H](Cc1ccc(O[Si](C)(C)C(C)(C)C)c(NC(=O)CCCN)c1)NC(=O)OC(C)(C)C]

(4R)-ethyl 5-(3-(4-(((benzyloxy)carbonyl)amino) butanamido)-4-((tert-butyldimethyl-silyl)oxy)phenyl)-4-((tert-butoxycarbonyl)amino)-2-methylpentanoate (0.35 g, 0.5 mmol) was dissolved in MeOH (5 ml), and Pd/C (10 wt %, 35 mg) was then added. The reaction mixture was stirred at r.t. under H₂ balloon overnight, then filtered through Celite and the filtrate was concentrated under reduced pressure to give the title product (0.22 g, 79% yield). ESI MS m/z: calcd for C₂₉H₅₂N₃O₆Si [M+H]⁺:566.35, found 566.35.

### Example 157. Synthesis of (2R,3S)-2,3-bis(((benzyloxy)carbonyl)amino)succinic Acid

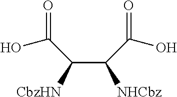 [SMILES: O=C(N[C@H](C(=O)O)[C@@H](NC(=O)OCc1ccccc1)C(=O)O)OCc1ccccc1]

To a solution of (2R,3S)-2,3-diaminosuccinic acid (4.03 g, 27.30 mmol) in the mixture of THE (250 ml) and NaH₂PO₄ (0.1 M, 250 ml, pH 8.0) was added benzyl carbonochloridate (15.0 g, 88.23 mmol) in 4 portions in 2 h. The mixture was stirred for another 6 h, concentrated and purified on SiO₂ column eluted with H₂O/CH₃CN (1:9) containing 1% formic acid to afford the title compound (8.63 g, 75% yield). MS ESI m/z calcd for C₂₀H₂₁N₂O₈ [M+H]⁺ 417.12, found 417.50.

### Example 158. Synthesis of (2R,3S)-bis(2,5-dioxopyrrolidin-1-yl) 2,3-bis(((benzyloxy)-carbonyl)amino)succinate

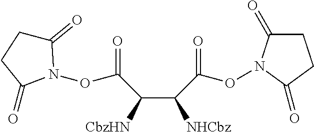 [SMILES: O=C(N[C@H](C(=O)ON1C(=O)CCC1=O)[C@@H](NC(=O)OCc1ccccc1)C(=O)ON1C(=O)CCC1=O)OCc1ccccc1]

To a solution of (2R,3S)-2,3-bis(((benzyloxy)carbonyl)amino)succinic acid (4.25 g, 10.21 mmol) in the mixture of DMA (70 ml) was added NHS (3.60 g, 31.30 mmol) and EDC (7.00 g, 36.65 mmol). The mixture was stirred for overnight, concentrated and purified on SiO₂ column eluted with EtOAc/CH₂Cl₂ (1:6) to afford the title compound (5.48 g, 88% yield). MS ESI m/z calcd for C₂₈H₂₇N₄O₁₂ [M+H]⁺ 611.15, found 611.45.

### Example 159. Synthesis of di-tert-butyl 4,4′-(((2R,3S)-2,3-bis(((benzyloxy)carbonyl)-amino)succinyl)bis(azanediyl))dibutanoate

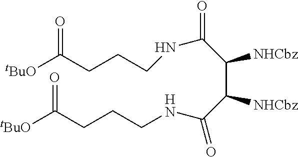 [SMILES: CC(C)(C)OC(=O)CCCNC(=O)[C@@H](NC(=O)OCc1ccccc1)[C@@H](NC(=O)OCc1ccccc1)C(=O)NCCCC(=O)OC(C)(C)C]

To a solution of (2R,3S)-2,3-bis(((benzyloxy)carbonyl)amino)succinic acid (4.25 g, 10.21 mmol) in the mixture of DMA (70 ml) was added tert-butyl 4-aminobutanoate (3.25 g, 20.42 mmol) and EDC (7.00 g, 36.65 mmol). The mixture was stirred for overnight, concentrated and purified on SiO₂ column eluted with EtOAc/CH₂Cl₂ (1:10) to afford the title compound (6.50 g, 91% yield). MS ESI m/z calcd for C₃₆H₅₁N₄O₁₀ [M+H]⁺ 699.35, found 699.55.

### Example 160. Synthesis of di-tert-butyl 4,4′-(((2R,3S)-2,3-diaminosuccinyl)-bis(azanediyl))dibutanoate

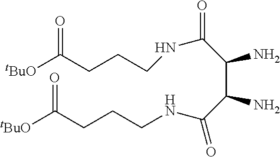 [SMILES: CC(C)(C)OC(=O)CCCNC(=O)[C@@H](N)[C@@H](N)C(=O)NCCCC(=O)OC(C)(C)C]

To a solution of di-tert-butyl 4,4′-(((2R,3S)-2,3-bis(((benzyloxy)carbonyl)amino)-succinyl)bis(azanediyl))dibutanoate (2.50 g, 3.58 mmol) in MeOH (100 mL) was added 10% Pd/C (0.30 g, 50% wet), the mixture was stirred under hydrogen atmosphere at room temperature for 18 h. Then the Pd/C was removed by filtration through celite and the filter bed was washed with MeOH (˜70 ml). The filtrate was concentrated to afford the product as yellow foam which was used in the next step without further purification (1.54 g, 100% yield). ESI: m/z: calcd for C₂₀H₃₉N₂O₆ [M+H]⁺: 431.28, found 431.50.

### Example 161. Synthesis of di-tert-butyl 4,4′-(((2R,3S)-2,3-bis(3-(2,5-dioxo-2,5-dihydro-1H-pyrrol-1-yl)propanamido)succinyl)bis(azanediyl))dibutanoate

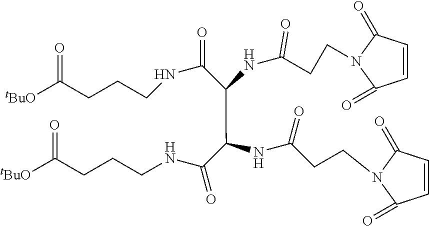 [SMILES: CC(C)(C)OC(=O)CCCNC(=O)[C@@H](NC(=O)CCN1C(=O)C=CC1=O)[C@@H](NC(=O)CCN1C(=O)C=CC1=O)C(=O)NCCCC(=O)OC(C)(C)C]

To a solution of 3-(2,5-dioxo-2,5-dihydro-1H-pyrrol-1-yl)propanoic acid (1.25 g, 7.39 mmol) in the mixture of DMA (60 ml) was added di-tert-butyl 4,4′-(((2R,3S)-2,3-diaminosuccinyl)-bis(azanediyl))dibutanoate (1.54 g, -3.57 mmol) and EDC (2.40 g, 12.56 mmol). The mixture was stirred for overnight, concentrated and purified on SiO₂ column eluted with EtOAc/CH₂Cl₂ (1:10) to afford the title compound (2.35 g, 90% yield). MS ESI m/z calcd for C₃₄H₄₉N₆O₁₂ [M+H]⁺ 733.33, found 733.60.

### Example 162. Synthesis of 4,4′-(((2R,3S)-2,3-bis(3-(2,5-dioxo-2,5-dihydro-1H-pyrrol-1-yl)propanamido)succinyl)bis(azanediyl))dibutanoic Acid

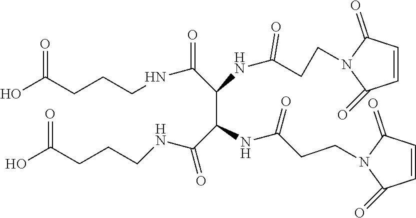 [SMILES: O=C(O)CCCNC(=O)[C@@H](NC(=O)CCN1C(=O)C=CC1=O)[C@@H](NC(=O)CCN1C(=O)C=CC1=O)C(=O)NCCCC(=O)O]

To a stirred solution of di-tert-butyl 4,4′-(((2R,3S)-2,3-bis(3-(2,5-dioxo-2,5-dihydro-1H-pyrrol-1-yl)propanamido)succinyl)bis(azanediyl))dibutanoate (2.30 g, 3.14 mmol) in 1,4-dioxane (20 ml) was added HCl (36%, 7.0 ml). The mixture was stirred for 30 min, diluted with toluene (20 ml), concentrated and purified on SiO₂ column eluted with MeOH/CH₂Cl₂ (1:10 to 1:4) to afford the title compound (1.69 g, 86% yield). MS ESI m/z calcd for C₂₆H₃₃N₆O₁₂ [M+H]⁺ 621.21, found 621.70.

### Example 163. Synthesis of di-tert-butyl 4,4′-(((2R,3S)-2,3-bis(2-(2,5-dioxo-2,5-dihydro-1H-pyrrol-1-yl)acetamido)succinyl)bis(azanediyl))dibutanoate

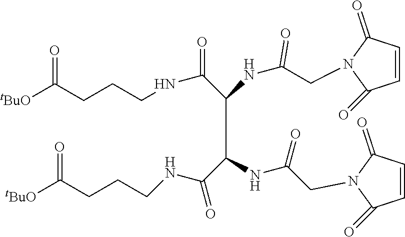 [SMILES: CC(C)(C)OC(=O)CCCNC(=O)[C@@H](NC(=O)CN1C(=O)C=CC1=O)[C@@H](NC(=O)CN1C(=O)C=CC1=O)C(=O)NCCCC(=O)OC(C)(C)C]

To a solution of 2-(2,5-dioxo-, -dihydro-1H-pyrrol-1-yl)acetic acid (1.12 g, 7.22 mmol) in the mixture of DMA (60 ml) was added di-tert-butyl 4,4′-(((2R,3S)-2,3-diaminosuccinyl)-bis(azanediyl))dibutanoate (1.54 g, -3.58 mmol) and EDC (2.40 g, 12.56 mmol). The mixture was stirred for overnight, concentrated and purified on SiO₂ column eluted with EtOAc/CH₂Cl₂ (1:10) to afford the title compound (2.29 g, 91% yield). MS ESI m/z calcd for C₃₂H₄₅N₆O₁₂ [M+H]⁺ 704.30, found 704.60.

### Example 164. Synthesis of 4,4′-(((2R,3S)-2,3-bis(2-(2,5-dioxo-2,5-dihydro-1H-pyrrol-1-yl)acetamido)succinyl)bis(azanediyl))dibutanoic Acid

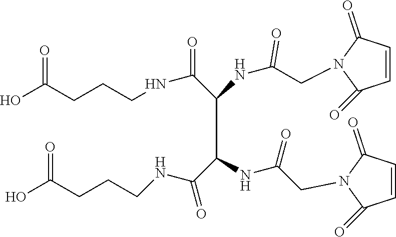 [SMILES: O=C(O)CCCNC(=O)[C@@H](NC(=O)CN1C(=O)C=CC1=O)[C@@H](NC(=O)CN1C(=O)C=CC1=O)C(=O)NCCCC(=O)O]

To a stirred solution of di-tert-butyl 4,4′-(((2R,3S)-2,3-bis(2-(2,5-dioxo-2,5-dihydro-1H-pyrrol-1-yl)acetamido)succinyl)bis(azanediyl))dibutanoate (2.20 g, 3.12 mmol) in 1,4-dioxane (20 ml) was added HCl (36%, 7.0 ml). The mixture was stirred for 30 min, diluted with toluene (20 ml), concentrated and purified on SiO₂ column eluted with MeOH/CH₂Cl₂ (1:10 to 1:4) to afford the title compound (1.69 g, 86% yield). MS ESI m/z calcd for C₂₄H₂₉N₆O₁₂ [M+H]⁺ 593.18, found 593.40.

### Example 165. Synthesis of bis(2,5-dioxopyrrolidin-1-yl) 4,4′-(((2R,3S)-2,3-bis(2-(2,5-dioxo-2,5-dihydro-1H-pyrrol-1-yl)acetamido)succinyl)bis(azanediyl))dibutanoate

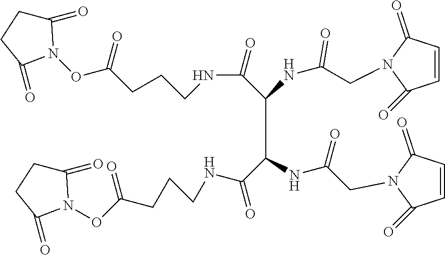 [SMILES: O=C(CN1C(=O)C=CC1=O)N[C@H](C(=O)NCCCC(=O)ON1C(=O)CCC1=O)[C@@H](NC(=O)CN1C(=O)C=CC1=O)C(=O)NCCCC(=O)ON1C(=O)CCC1=O]

To a solution of 4,4′-(((2R,3S)-2,3-bis(2-(2,5-dioxo-2,5-dihydro-1H-pyrrol-1-yl)acetamido)succinyl)bis(azanediyl))dibutanoic acid (1.10 g, 1.85 mmol) in the mixture of DMA (30 ml) was added NHS (1-hydroxypyrrolidine-2,5-dione) (0.55 g, 4.78 mmol) and EDC (1.25 g, 6.54 mmol). The mixture was stirred for overnight, concentrated and purified on SiO₂ column eluted with EtOAc/CH₂Cl₂ (1:10) to afford the title compound (1.30 g, 90% yield). MS ESI m/z calcd for C₃₂H₃₅N₈O₁₆ [M+H]⁺ 787.21, found 787.60.

### Example 166. Synthesis of (2S,3S)-2,3-bis(2,5-dioxo-2,5-dihydro-1H-pyrrol-1-yl)succinic Acid

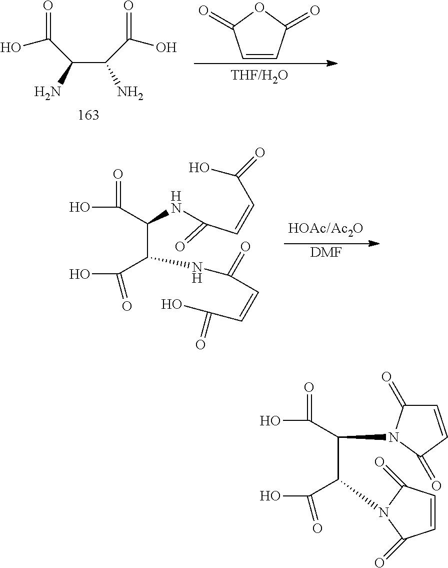

(2R,3R)-2,3-diaminosuccinic acid (5.00 g, 33.77 mmol) in the mixture of THF/H₂O/DIPEA (125 ml/125 ml/2 ml) was added maleic anhydride (6.68 g, 68.21 mmol). The mixture was stirred for overnight, evaporated to afforded (2S,3S)-2,3-bis((Z)-3-carboxyacrylamido)succinic acid (11.05 g, 99% yield) as a white solid. MS ESI m/z calcd for C₁₂H₁₃N₂O₁₀ [M+H]⁺ 345.05, found 345.35.

(2S,3S)-2,3-bis((Z)-3-carboxyacrylamido)succinic acid (11.05 g, 33.43 mmol) in a mixture solution of HOAc (70 ml), DMF (10 ml) and toluene (50 ml) was added acetic anhydride (30 ml). The mixture was stirred for 2 h, reflux with Dean-Stark Trap at 100° C. for 6 h, concentrated, co-evaporated with EtOH (2×40 ml) and toluene (2×40 ml), and purified on SiO₂ column eluted with H₂O/CH₃CN (1:10) to afford the title compound (8.10 g, 78% yield). MS ESI m/z calcd for Cl₂H₉N₂O₈[M+H]⁺ 309.03, found 309.50.

### Example 167. Synthesis of (2S,3S)-bis(2,5-dioxopyrrolidin-1-yl) 2,3-bis(2,5-dioxo-2,5-dihydro-1H-pyrrol-1-yl)succinate

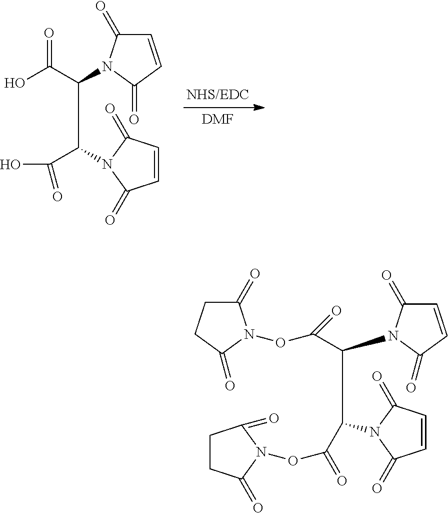

To a solution of (2S,3S)-2,3-bis(2,5-dioxo-2,5-dihydro-1H-pyrrol-1-yl)succinic acid (4.00 g, 12.98 mmol) in the mixture of DMF (70 ml) was added NHS (3.60 g, 31.30 mmol) and EDC (7.00 g, 36.65 mmol). The mixture was stirred for overnight, concentrated and purified on SiO₂ column eluted with EtOAc/CH₂Cl₂ (1:6) to afford the title compound (5.79 g, 89% yield, ˜96% pure by HPLC). MS ESI m/z calcd for C₂₀H₁₅N₄O₁₂ [M+H]⁺ 503.06, found 503.60.

### Example 168. Synthesis of (4R)-tert-butyl 5-(3-(4-(((benzyloxy)carbonyl)amino)-butanamido)-4-hydroxyphenyl)-4-((tert-butoxycarbonyl)amino)-2-methylpentanoate

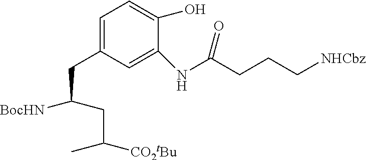 [SMILES: CC(C[C@H](Cc1ccc(O)c(NC(=O)CCCNC(=O)OCc2ccccc2)c1)NC(=O)OC(C)(C)C)C(=O)OC(C)(C)C]

HATU (39.9 g, 105 mmol) was added to a solution of 4-(((benzyloxy)carbonyl)amino) butanoic acid (26.1 g, 110 mmol) in DMF (300 mL). After stirring at r.t. for 30 min, the mixture was added to a solution of (4R)-tert-butyl 5-(3-amino-4-hydroxyphenyl)-4-((tert-butoxycarbonyl)amino)-2-methylpentanoate (39.4 g, 100 mmol) and TEA (20.2 g, 200 mmol) in DMF (300 mL).The resulting mixture was stirred at r.t. for 2 h. Water was then added, extracted with EtOAc, the organic layer was washed with brine, dried over Na₂SO₄. Purification by column chromatography (20% to 70% EA/PE) yielded the title product as a white solid (45 g, 73% yield). ESI m/z calcd for C₃₃H₄₈N₃O₈ [M+H]⁺: 614.34, found 614.15.

### Example 169. Synthesis of (4R)-tert-butyl 5-(3-(4-aminobutanamido)-4-hydroxyphenyl)-4-((tert-butoxycarbonyl)amino)-2-methylpentanoate

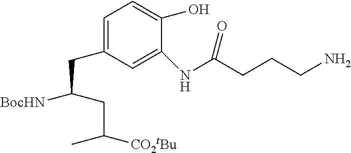 [SMILES: CC(C[C@H](Cc1ccc(O)c(NC(=O)CCCN)c1)NC(=O)OC(C)(C)C)C(=O)OC(C)(C)C]

(4R)-Tert-butyl 5-(3-(4-(((benzyloxy)carbonyl)amino)-butanamido)-4-hydroxyphenyl)-4-((tert-butoxycarbonyl)amino)-2-methylpentanoate (100 g, 163 mmol) was dissolved in methanol (500 mL) and hydrogenated (1 atm) with Pd/C catalyst (10 wt %, 10 g) at r.t. overnight. The catalyst was filtered off and the filtrate were concentrated under reduced pressure to afford the title compound (75.8 g, 97% yield) as a brown foamy solid. ¹H NMR (400 MHz, CDCl3) δ 7.11 (s, 1H), 6.83 (d, J=10.3 Hz, 2H), 5.04-4.52 (m, 6H), 3.90-3.56 (m, 1H), 2.81 (d, J=5.3 Hz, 2H), 2.63 (dd, J=12.5, 6.1 Hz, 2H), 2.54-2.26 (dd, J=14.0, 7.6 Hz, 4H), 1.94-1.64 (m, 3H), 1.44-1.36 (m, 18H), 1.08 (d, J=6.9 Hz, 3H). ESI m/z calcd for C₂₅H₄₂N₃O₆ [M+H]⁺: 480.30, found 480.59.

### Example 170. Synthesis of (4R)-tert-butyl 5-(3-((S)-37-(((benzyloxy)carbonyl)amino)-31,38-dioxo-2,5,8,11,14,17,20,23,26,29-decaoxa-32,39-diazatritetracontanamido)-4-hydroxyphenyl)-4-((tert-butoxycarbonyl)amino)-2-methylpentanoate

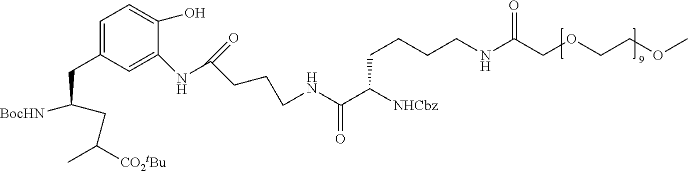

To a solution of (4R)-tert-butyl 5-(3-(4-aminobutanamido)-4-hydroxyphenyl)-4-((tert-butoxycarbonyl)amino)-2-methylpentanoate (130 g, 174 mmol, 1.1 eq.) in DMF (500 mL) were added TEA (66 mL, 474 mmol, 3 eq.) and HATU (72 g, 190 mmol, 1.2 eq.) in sequence at 0° C. Then the reaction mixture was warmed to r.t and stirred for 2 h. A solution of (S)-37-(((benzyloxy)carbonyl)amino)-31-oxo-2,5,8,11,14,17,20,23,26,29-decaoxa-32-azaoctatriacontan-38-oic acid (75.8 g, 158 mmol, 1.0 eq) in DMF (500 mL) was added to the above solution at 0° C., and the reaction mixture was stirred at RT for 1 h. The reaction mixture was poured into water (4 L), the aqueous layer was extracted with EtOAc (3×500 mL), and the organic layers were combined and washed with brine (2 L), dried over Na₂SO₄, concentrated and the crude title product (190 g) was used in the next step directly. ESI: m/z: calcd for C₆₀H₁₀₀N₅O₂₀ [M+H]⁺: 1210.69, found 1210.69.

### Example 171. Synthesis of (4R)-tert-butyl 5-(3-((S)-37-amino-31,38-dioxo-2,5,8,11,14,17,20,23,26,29-decaoxa-32,39-diazatritetracontanamido)-4-hydroxyphenyl)-4-((tert-butoxycarbonyl)amino)-2-methylpentanoate

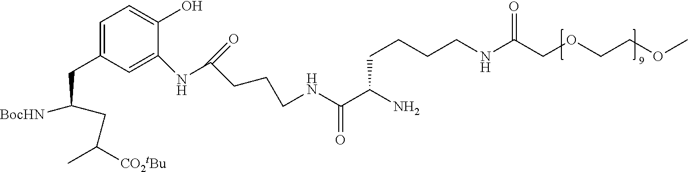

The crude product of (4R)-tert-butyl 5-(3-((S)-37-(((benzyloxy)carbonyl)amino)-31,38-dioxo-2,5,8,11,14,17,20,23,26,29-decaoxa-32,39-diazatritetracontanamido)-4-hydroxyphenyl)-4-((tert-butoxycarbonyl)amino)-2-methylpentanoate (190 g) was dissolved in methanol (900 mL) and hydrogenated (1 atm) with Pd/C catalyst (10 wt %, 19 g) at r.t. overnight. The catalyst was filtered off and the filtrate were concentrated under reduced pressure, and the crude compound was purified by SiO₂ column with a gradient of DCM/MeOH to give the title product (105 g, 62% yield over two steps) as a brown oil. ESI m/z calcd for C₅₂H₉₅N₅O₁₈ [M+H]⁺: 1077.65, found 1077.65.

### Example 172. Synthesis of 2-((6S,9S,12R,14R)-9-((S)-sec-butyl)-14-hydroxy-6,12-diisopropyl-2,2,5,11-tetramethyl-4,7,10-trioxo-3-oxa-5,8,11-triazatetradecan-14-yl)thiazole-4-carboxylic Acid

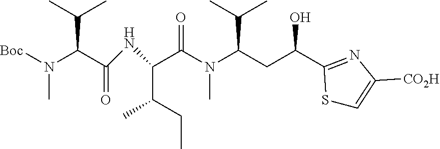 [SMILES: CC[C@H](C)[C@H](NC(=O)[C@H](C(C)C)N(C)C(=O)OC(C)(C)C)C(=O)N(C)[C@H](C[C@@H](O)c1nc(C(=O)O)cs1)C(C)C]

To a solution of Boc-Me-L-Val-OH (33 mg, 0.14 mmol) in EtOAc was added pentafluorophenol (39 mg, 0.21 mmol) and DCC (32 mg, 0.154 mmol). The reaction mixture was stirred at r.t. for 16 h and then filtered over a Celite pad, with washing of the pad with EtOAc. The filtrate was concentrated and re-dissolved in DMA (2 mL), and then 2-((1R,3R)-3-((2S,3S)-2-amino-N,3-dimethylpentanamido)-1-hydroxy-4-methylpentyl)thiazole-4-carboxylic acid (52 mg, 0.14 mmol) and DIPEA (48.5 μL, 0.28 mmol) were added. The reaction mixture was stirred at r.t. for 24 h and then concentrated and purified by reverse phase HPLC (C₁₈ column, 10-100% acetonitrile/water) to afford the title compound (40.2 mg, 49% yield). ESI MS m/z: calcd for C₂₈H₄₉N₄O₇S [M+H]⁺: 585.32, found 585.32.

### Example 173. Synthesis of 2-((6S,9S,12R,14R)-9-((S)-sec-butyl)-6,12-di-isopropyl-2,2,5,11-tetramethyl-4,7,10,16-tetraoxo-3,15-dioxa-5,8,11-triazaheptadecan-14-yl)thiazole-4-carboxylic Acid

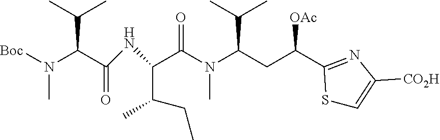 [SMILES: CC[C@H](C)[C@H](NC(=O)[C@H](C(C)C)N(C)C(=O)OC(C)(C)C)C(=O)N(C)[C@H](C[C@@H](OC(C)=O)c1nc(C(=O)O)cs1)C(C)C]

2-((6S,9S,12R,14R)-9-((S)-sec-butyl)-14-hydroxy-6,12-diisopropyl-2,2,5,11-tetramethyl-4,7,10-trioxo-3-oxa-5,8,11-triazatetradecan-14-yl)thiazole-4-carboxylic acid (40 mg, 0.069 mmol) was dissolved in pyridine (8 mL), to which acetic anhydride (20.4 mg, 0.2 mmol) was added at 0° C. and the reaction was allowed to warm to r.t. and stirred overnight. The mixture was concentrated and the residue purified by SiO₂ column chromatography with a gradient of DCM/MeOH to give the title product (48.1 mg, ˜100% yield). ESI MS m/z: calcd for C₃₀H₅₁N₄O₈S [M+H]⁺ 627.33, found 627.33.

### Example 174. Synthesis of (4R)-4-(2-((6S,9S,12R,14R)-9-((S)-sec-butyl)-6,12-diisopropyl-2,2,5,11-tetramethyl-4,7,10,16-tetraoxo-3,15-dioxa-5,8,11-triazaheptadecan-14-yl)thiazole-4-carboxamido)-2-methyl-5-phenylpentanoic Acid

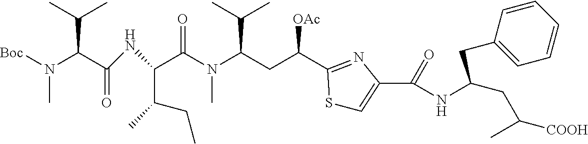 [SMILES: CC[C@H](C)[C@H](NC(=O)[C@H](C(C)C)N(C)C(=O)OC(C)(C)C)C(=O)N(C)[C@H](C[C@@H](OC(C)=O)c1nc(C(=O)N[C@@H](Cc2ccccc2)CC(C)C(=O)O)cs1)C(C)C]

To a solution of 2-((6S,9S,12R,14R)-9-((S)-sec-butyl)-6,12-di-isopropyl-2,2,5,11-tetramethyl-4,7,10,16-tetraoxo-3,15-dioxa-5,8,11-triazaheptadecan-14-yl)thiazole-4-carboxylic acid (48.1 mg, 0.077 mmol) in EtOAc was added pentafluorophenol (21.2 mg, 0.115 mmol) and DCC (17.4 mg, 0.085 mmol). The reaction mixture was stirred at r.t. for 16 h and then filtered over a Celite pad, with washing of the pad with EtOAc. The filtrate was concentrated and re-dissolved in DMA (4 mL), and then (4R)-4-amino-2-methyl-5-phenylpentanoic acid (20.7 mg, 0.1 mmol) and DIPEA (26.8 μL, 0.154 mmol) were added. The reaction mixture was stirred at r.t. for 24 h and then concentrated and purified by reverse phase HPLC (C₁₈ column, 10-100% acetonitrile/water) to afford the title compound (63 mg, -100% yield). ESI MS m/z: calcd for C₄₂H₆₆N₅O₉S [M+H]⁺ 816.45, found 816.45.

### Example 175. Synthesis of (4R)-4-(2-((3S,6S,9R,11R)-6-((S)-sec-butyl)-3,9-diisopropyl-8-methyl-4,7,13-trioxo-12-oxa-2,5,8-triazatetradecan-11-yl)thiazole-4-carboxamido)-2-methyl-5-phenylpentanoic Acid Hydrochloride Salt

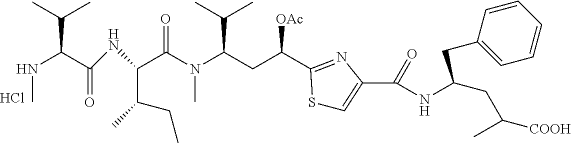 [SMILES: Cl ; CC[C@H](C)[C@H](NC(=O)[C@@H](NC)C(C)C)C(=O)N(C)[C@H](C[C@@H](OC(C)=O)c1nc(C(=O)N[C@@H](Cc2ccccc2)CC(C)C(=O)O)cs1)C(C)C]

(4R)-4-(2-((6S,9S,12R,14R)-9-((S)-sec-butyl)-6,12-diisopropyl-2,2,5,11-tetramethyl-4,7,10,16-tetraoxo-3,15-dioxa-5,8,11-triazaheptadecan-14-yl)thiazole-4-carboxamido)-2-methyl-5-phenylpentanoic acid (60 mg, 0.073 mmol) in ethyl acetate (3 ml) and hydrogen chloride (0.8 ml, 12 M). The mixture was stirred for 30 min and diluted with toluene (5 ml) and dioxane (5 ml). The mixture was evaporated and co-evaporated with dioxane (5 ml) and toluene (5 ml) to dryness. The yielded crude title product (57.1 mg, 103% yield) was used for the next step without further purification. ESI MS m/z: calcd for C₃₇H₅₈N₅O₇S [M+H]⁺ 716.40, found 716.60.

### Example 176. Synthesis of (4R)-tert-butyl-5-(3-(2-(2-(((benzyloxy)carbonyl)amino)-propanamido)acetamido)-4-hydroxyphenyl)-4-((tert-butoxycarbonyl)amino)-2-methylpentanoate

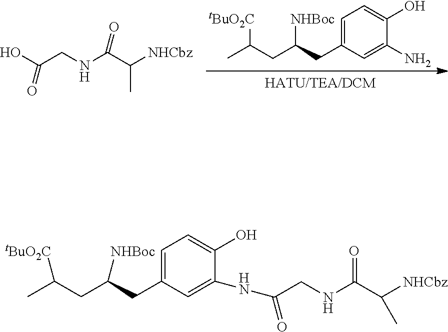 [SMILES: CC(NC(=O)OCc1ccccc1)C(=O)NCC(=O)O ; CC(C[C@H](Cc1ccc(O)c(N)c1)NC(=O)OC(C)(C)C)C(=O)OC(C)(C)C ; CC(C[C@H](Cc1ccc(O)c(NC(=O)CNC(=O)C(C)NC(=O)OCc2ccccc2)c1)NC(=O)OC(C)(C)C)C(=O)OC(C)(C)C]

2-(2-(((benzyloxy)carbonyl)amino)propanamido)acetic acid (0.2 g, 0.7 mmol), (4R)-tert-butyl-5-(3-amino-4-hydroxyphenyl)-4-((tert-butoxycarbonyl)amino)-2-methylpentanoate (0.19 g, 0.48 mmol), and HATU (0.18 g, 0.48 mmol) were dissolved in DCM (20 ml), followed by addition of TEA (134 ul, 0.96 mmol). The reaction mixture was stirred at RT overnight, concentrated under reduced pressure and the residue was purified on SiO₂ column to give the title product (0.3 g, 95%). ESI: m/z: calcd for C₃₄H₄₉N₄O₉ [M+H]⁺:657.34, found 657.34.

### Example 177. Synthesis of (4R)-tert-butyl-5-(3-(2-(2-aminopropanamido)acetamido)-4-hydroxyphenyl)-4-((tert-butoxycarbonyl)amino)-2-methylpentanoate

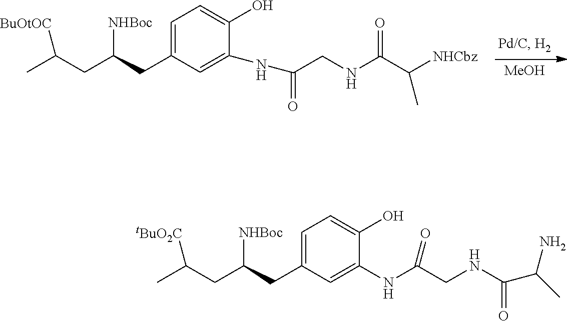

In a hydrogenation bottle, Pd/C (0.1 g, 33 wt %, 50% wet) was added to a solution of (4R)-tert-butyl-5-(3-(2-(2-(((benzyloxy)carbonyl)amino)propanamido)acetamido)-4-hydroxyphenyl)-4-((tert-butoxycarbonyl)amino)-2-methylpentanoate (0.3 g, 0.46 mmol) in MeOH (10 mL). The mixture was shaken overnight under 1 atm H₂ then filtered through Celite (filter aid), and the filtrate was concentrated to afford the title compound (0.21 g, 87%) used for next step without further purification. ESI: m/z: calcd for C₂₆H₄₃N₄O₇ [M+H]⁺:523.31, found 523.31.

### Example 178. Synthesis of 2-carboxy-N,N,N-trimethylpropan-2-aminium bromide

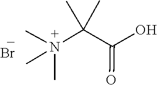

To a solution of 2-bromo-2-methylpropanoic acid (3.00 g, 17.9 mmol) in THE (30 mL) was added trimethylamine (1M solution in THF, 17.9 mL, 35.9 mmol). The reaction mixture was stirred overnight at r.t. The precipitate was collected by filtration and washed with EA to give the title compound (4.00 g, theoretical yield) as a white solid. ESI m/z calcd for C₇H₁₆NO₂ [M+H]⁺: 146, found 146.

### Example 179. Synthesis of N,N,N,2-tetramethyl-1-oxo-1-(perfluorophenoxy)propan-2-aminium bromide

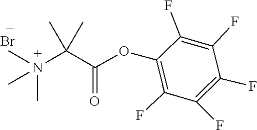

To a solution of 2-carboxy-N,N,N-trimethylpropan-2-aminium bromide (1.55 g, 6.9 mmol) and PFP (2.50 g, 13.8 mmol) in DCM (20 mL) was added DCC (2.80 g, 13.8 mmol). The reaction mixture was stirred at r.t. overnight. The reaction was filtered and the filtrate was concentrated under vacuum to give the title compound as a colorless oil, which was used directly in the next step. ESI m: calcd for C₁₃H₁₅F₅NO₂ [M+H]⁺: 312, found 312.

### Example 180. Synthesis of (5R,7R,10S)-10-(sec-butyl)-5-(4-(ethoxycarbonyl)thiazol-2-yl)-3,3-diethyl-7-isopropyl-N,N,N,8,13-pentamethyl-9,12-dioxo-4-oxa-8,11-diaza-3-silatetradecan-13-aminium

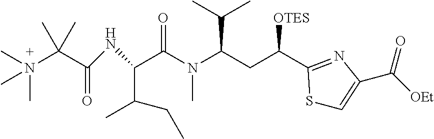

To a solution of ethyl 2-((1R,3R)-3-((2S)-2-amino-N,3-dimethylpentanamido)-4-methyl-1-((triethylsilyl)oxy)pentyl)thiazole-4-carboxylate (1.78 g, 3.4 mmol) and N,N,N,2-tetramethyl-1-oxo-1-(perfluorophenoxy)propan-2-aminium bromide (6.9 mmol) in DMF (20 mL) was added DIPEA (1.8 mL, 10.4 mmol) at 0° C. The reaction mixture was warmed to r.t. and stirred for 1h, then concentrated under vacuum and purified by silica column (100:1 to 5:1 DCM/MeOH) to give the title compound (1.20 g, 54% yield) as a foamy solid. ESI m/z calcd for C₃₂H₆₁N₄O₅SSi [M+H]⁺: 642, found 642.

### Example 181. Synthesis of 1-(((2S)-1-(((1R,3R)-1-(4-(ethoxycarbonyl)thiazol-2-yl)-1-hydroxy-4-methylpentan-3-yl)(methyl)amino)-3-methyl-1-oxopentan-2-yl)amino)-N,N,N,2-tetramethyl-1-oxopropan-2-aminium

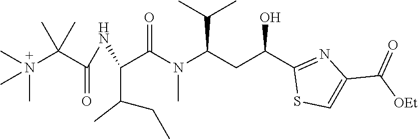

(5R,7R,10S)-10-(sec-butyl)-5-(4-(ethoxycarbonyl)thiazol-2-yl)-3,3-diethyl-7-isopropyl-N,N,N,8,13-pentamethyl-9,12-dioxo-4-oxa-8,11-diaza-3-silatetradecan-13-aminium (1.20 g, 1.86 mmol) was dissolved in AcOH/THF/H₂O (v/v/v 3:1:1, 20 mL) and stirred overnight. The reaction was then concentrated under vacuum, and used for the next step without further purification. ESI m/z calcd for C₂₆H₄₇N₄O₅S [M+H]⁺: 527, found 527.

### Example 182. Synthesis of 1-(((2S)-1-(((1R,3R)-1-(4-carboxythiazol-2-yl)-1-hydroxy-4-methylpentan-3-yl)(methyl)amino)-3-methyl-1-oxopentan-2-yl)amino)-N,N,N,2-tetramethyl-1-oxopropan-2-aminium

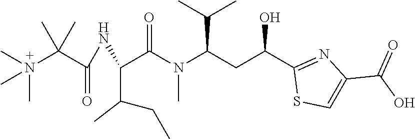

To a solution of 1-(((2S)-1-(((1R,3R)-1-(4-(ethoxycarbonyl)thiazol-2-yl)-1-hydroxy-4-methylpentan-3-yl)(methyl)amino)-3-methyl-1-oxopentan-2-yl)amino)-N,N,N,2-tetramethyl-1-oxopropan-2-aminium (1.86 mmol) in 1,4-dioxane (10 mL) was added 1N NaOH (9.3 mL). And the reaction mixture was stirred for 2 h and concentrated under vacuum. The residue was diluted with water (10 mL) and 1N HCl was added to adjust pH to ˜4. The mixture was concentrated under vacuum to give the title compound as a white solid. ESI m/z calcd for C₂₄H₄₃N₄O₅S [M+H]⁺: 499, found 499.

### Example 183. Synthesis of 1-(((2S)-1-(((1R,3R)-1-acetoxy-1-(4-carboxythiazol-2-yl)-4-methylpentan-3-yl)(methyl)amino)-3-methyl-1-oxopentan-2-yl)amino)-N,N,N,2-tetramethyl-1-oxopropan-2-aminium

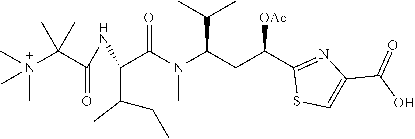

To a solution of 1-(((2S)-1-(((1R,3R)-1-(4-carboxythiazol-2-yl)-1-hydroxy-4-methylpentan-3-yl)(methyl)amino)-3-methyl-1-oxopentan-2-yl)amino)-N,N,N,2-tetramethyl-1-oxopropan-2-aminium (1.86 mmol) in pyridine (10 mL) was added acetic anhydride (884 μL, 9.36 mmol) at 0° C. Then the reaction mixture was warmed to r.t. and stirred overnight. The reaction was concentrated under vacuum and then diluted with H₂O (20 mL) and washed with EA (3×10 mL). The aqueous layer was concentrated under vacuum to give the title compound as a yellow solid. ESI m/z calcd for C₂₆H₄₅N₄O₆S [M+H]⁺: 541, found 541.

### Example 184. Synthesis of 1-(((2S)-1-(((1R,3R)-1-acetoxy-4-methyl-1-(4-((perfluorophenoxy)carbonyl)thiazol-2-yl)pentan-3-yl)(methyl)amino)-3-methyl-1-oxopentan-2-yl)amino)-N,N,N,2-tetramethyl-1-oxopropan-2-aminium

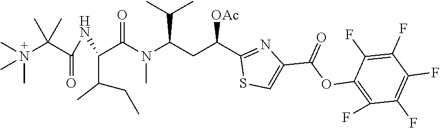

To a solution of 1-(((2S)-1-(((1R,3R)-1-acetoxy-1-(4-carboxythiazol-2-yl)-4-methylpentan-3-yl)(methyl)amino)-3-methyl-1-oxopentan-2-yl)amino)-N,N,N,2-tetramethyl-1-oxopropan-2-aminium (150 mg, 0.277 mmol) and pentafluorophenol (76.5 mg, 0.415 mmol) in DCM (2 mL) was added EDCI (63.7 mg, 0.33 mmol). The reaction mixture was stirred for 3 h and concentrated under vacuum to give the title compound as a yellow oil. ESI m/z calcd for C₃₂H₄₄F₅N₄O₆S [M+H]⁺:707, found 707.

### Example 185. Synthesis of (S)-4-isopropyl-3-propionyloxazolidin-2-one

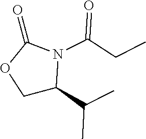

To a solution of (S)-4-isopropyloxazolidin-2-one (400 g, 3.09 mol, 1.0 eq.) in anhydrous THF (8 L) at about −70° C. was added n-BuLi (2.5 M in hexanes, 1.36 L, 3.4 mol, 1.1 eq.) under N₂. The mixture was stirred at −70° C. for 1 h, and then propionyl chloride (315 g, 3.4 mol, 1.1 eq.) was added slowly. After the addition was completed, the mixture was stirred at −70° C. for another 1 h, and gradually warmed to r.t. The reaction mixture was added to ice-cold saturated ammonium chloride solution (7 L) and extracted with EtOAc (3×2 L). The combined organic layers were washed with water (2 L) and brine (2 L), dried over anhydrous Na₂SO₄, filtered, concentrated and purified by column chromatography (3 kg silica gel, pure petroleum ether to 5:1 petroleum ether/EtOAc) to give the title compound as a colorless oil (500 g, 87% yield). MS ESI m/z calcd for C₉H₁₆NO₃ [M+H]⁺ 186.10, found 186.10.

### Example 186. Synthesis of (S)-methyl 3-(4-(benzyloxy)phenyl)-2-((tert-butoxycarbonyl)amino)propanoate

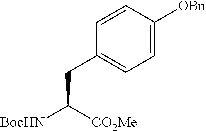 [SMILES: COC(=O)[C@H](Cc1ccc(OCc2ccccc2)cc1)NC(=O)OC(C)(C)C]

To a mixture of Boc-L-Tyr-OMe 900 g, 3.05 mol, 1.0 eq.), K₂CO₃ (632 g, 4.58 mol, 1.5 eq.) and KI (20 g, 0.150 mol, 0.05 eq.) in acetonitrile (3 L) was added benzyl bromide (547 g, 3.20 mol, 1.05 eq.) slowly. The mixture was then refluxed and monitored by TLC. After 4h, the reaction was cooled to r.t. and filtered. The filtrate was concentrated and diluted with water (3 L) and EtOAc (3.5 L), the organic phase was separated and the aqueous phase extracted with EtOAc (2×1.5 L). The combined organic layers were washed with brine (2×3 L), dried over anhydrous Na₂SO₄, filtered, concentrated. The crude products from 4 batches of 900 g and one batch of 400 g starting material were combined and weighed 5.4 kg, and then triturated with petroleum ether in 18 batches (4 L petroleum ether per batch). The solid was collected and filtrate was concentrated and purified by SiO₂ column chromatography (4:1 hexanes/EtOAc). All crops were combined to give the title compound total 4.85 kg of white solid (93% yield). ¹H NMR (500 MHz, CDCl₃) δ 7.43 (d, J=7.0 Hz, 2H), 7.38 (t, J=7.4 Hz, 2H), 7.32 (t, J=7.2 Hz, 1H), 7.04 (d, J=8.5 Hz, 2H), 6.91 (d, J=8.6 Hz, 2H), 5.04 (s, 2H), 4.55 (d, J=6.9 Hz, 1H), 3.71 (s, 3H), 3.03 (qd, J=14.0, 5.8 Hz, 2H), 1.43 (s, 9H). ESI: m/z: calcd for C₂₂H₂₈NO₅ [M+H]⁺: 386.19, found 386.19.

### Example 187. Synthesis of (S)-tert-butyl (1-(4-(benzyloxy)phenyl)-3-oxopropan-2-yl)carbamate

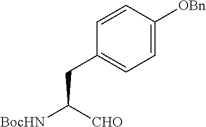 [SMILES: CC(C)(C)OC(=O)N[C@H](C=O)Cc1ccc(OCc2ccccc2)cc1]

To a solution of (S)-methyl 3-(4-(benzyloxy)phenyl)-2-((tert-butoxycarbonyl)amino)propanoate (288 g, 0.74 mol, 1.0 eq.) in anhydrous dichloromethane (2 L) at −78° C. was added DIBAL (1.5 M in toluene, 1.0 L, 2.0 eq.) slowly. After the addition was completed, the stirring was continued for 2 h. And the reaction mixture was poured onto ice water (2 L). 2N HCl (2 L) was added to dissolve the formed white precipitate. The organic phase was separated and aqueous phase extracted with dichloromethane (2×500 mL). The combined organic phase was washed with 2 N HCl (500 mL) and water (500 mL), dried over anhydrous Na₂SO₄, filtered and concentrated. The crude product was dissolved in dichloromethane (1 L) and loaded onto a collum (1 kg silica gel) and eluted with dichloromethane. The elution solution was concentrated and trituration with PE/EtOAc to give white solid of the title compound (152 g, 57% yield). 1H NMR (500 MHz, CDCl3) δ 9.65 (s, 1H), 7.45 (d, J=7.1 Hz, 2H), 7.41 (t, J=7.4 Hz, 2H), 7.35 (t, J=7.1 Hz, 1H), 7.11 (d, J=8.6 Hz, 2H), 6.95 (d, J=8.6 Hz, 2H), 5.07 (s, 2H), 4.42 (dd, J=12.4, 6.1 Hz, 1H), 3.09 (d, J=6.2 Hz, 2H), 1.46 (s, 9H). ESI: m/z: calcd for C₂₁H₂₆NO₄ [M+H]⁺: 356.18, found 356.19. The over-reduced product alcohol was also collected from the collum (65 g).

### Example 188. Synthesis of tert-butyl ((2S,3S,4S)-1-(4-(benzyloxy)phenyl)-3-hydroxy-5-((S)-4-isopropyl-2-oxooxazolidin-3-yl)-4-methyl-5-oxopentan-2-yl)carbamate

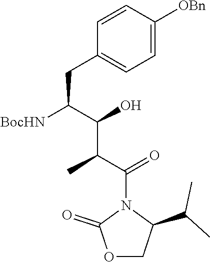 [SMILES: CC(C)[C@H]1COC(=O)N1C(=O)[C@@H](C)[C@H](O)[C@H](Cc1ccc(OCc2ccccc2)cc1)NC(=O)OC(C)(C)C]

To a solution of (S)-4-isopropyl-3-propionyloxazolidin-2-one (92.6 g, 0.50 mol, 1.1 eq.) in anhydrous dichloromethane (1.5 L) was added DIPEA (70.5 g, 0.54 mol, 1.2 eq.) at r.t. The mixture was cooled to −10° C. and n-Bu₂BOTf (1.0 M in dichloromethane, 500 mL, 1.1 eq.) was added under N₂. The temperature of reaction mixture was maintained below 0° C. during addition. The reaction was then stirred at 0° C. for 1 h and then cooled to −78° C., to which a solution of (S)-4-isopropyl-3-propionyloxazolidin-2-one (161 g, 0.45 mol, 1.0 eq.) in dichloromethane (1 L) was added dropwise. The temperature of reaction mixture was maintained below 0° C. during addition. The mixture was stirred at −78° C. for 2 h and then warmed slowly to room temperature and stirred overnight. PBS (0.1M, pH 7.0, 2 L) was added. After phase separation, the aqueous phase was further extracted with dichloromethane (2×500 mL). The combined organic layers were dried over anhydrous Na₂SO₄, filtered and concentrated. The crude product was re-dissolved in methanol (2 L) and cooled to 0° C., then treated with H₂O₂(30% aqueous solution, 500 mL) and stirred for 1 h. The methanol was removed by rotary evaporation and water (3 L) was added. The resulting mixture was extracted with dichloromethane (3×800 mL). The combined organic layers were washed with water (500 mL), saturated NaHCO₃ (500 mL) and brine (500 mL), dried over anhydrous Na₂SO₄, filtered and concentrated. The residue was mixed with 400 g silica gel and purified by column chromatography (2 kg silica gel, pure PE to 5:1 PE/EtOAc) to give the title compound as a foamy solid (150 g, 61% yield). ¹H NMR (400 MHz, CDCl₃) δ 7.36 (ddd, J=24.2, 14.2, 7.1 Hz, 5H), 7.12 (d, J=8.4 Hz, 2H), 6.90 (d, J=8.5 Hz, 2H), 5.02 (s, 2H), 4.69 (d, J=9.0 Hz, 1H), 4.45 (d, J=4.1 Hz, 1H), 4.33 (t, J=8.4 Hz, 1H), 4.15 (d, J=8.6 Hz, 1H), 3.90 (dd, J=16.6, 8.0 Hz, 1H), 3.85-3.77 (m, 2H), 2.81 (d, J=7.6 Hz, 2H), 2.27 (dd, J=11.4, 6.7 Hz, 1H), 1.35 (s, 9H), 0.89 (dd, J=14.3, 6.9 Hz, 6H). MS ESI m/z calcd for C₃₀H₄₁N₂O₇ [M+H]⁺ 541.28, found 541.30.

### Example 189. Synthesis of O-((2S,3S,4S)-5-(4-(benzyloxy)phenyl)-4-((tert-butoxycarbonyl)amino)-1-((S)-4-isopropyl-2-oxooxazolidin-3-yl)-2-methyl-1-oxopentan-3-yl) 1H-imidazole-1-carbothioate

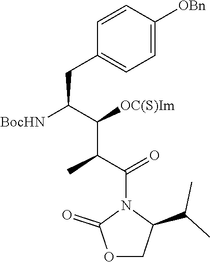

A mixture of tert-butyl ((2S,3S,4S)-1-(4-(benzyloxy)phenyl)-3-hydroxy-5-((S)-4-isopropyl-2-oxooxazolidin-3-yl)-4-methyl-5-oxopentan-2-yl)carbamate (200 g, 0.37 mol, 1.0 eq.) and 1,1′-thiocarbonyldiimidazole (198 g, 1.11 mol, 3.0 eq.) in anhydrous THE (3.5 L) was refluxed for 8 h. After which, more 1,1′-thiocarbonyldiimidazole (65 g, 0.37 mol, 1.0 eq.) was added and the mixture was refluxed overnight. THE was removed by rotary evaporation and the residue was mixed with 500 g silica gel and purified by column chromatography (2 kg silica gel, pure PE to 3:1 PE/EtOAc) to give the title compound as a yellow foam (170 g, 83% yield). ¹H NMR (400 MHz, CDCl₃) δ 8.41 (s, 1H), 7.67 (s, 1H), 7.36 (dt, J=16.0, 6.9 Hz, 6H), 7.09 (s, 1H), 7.05 (d, J=8.4 Hz, 2H), 6.86 (d, J=8.4 Hz, 2H), 6.32 (d, J=9.5 Hz, 1H), 5.01 (s, 2H), 4.56-4.43 (m, 2H), 4.32 (ddd, J=16.2, 15.6, 7.8 Hz, 3H), 4.19 (d, J=8.7 Hz, 1H), 2.96 (dd, J=14.6, 4.4 Hz, 1H), 2.49 (dd, J=14.5, 10.5 Hz, 1H), 2.29 (td, J=13.4, 6.7 Hz, 1H), 1.73 (s, 1H), 1.29 (s, 9H), 0.91 (dd, J=13.9, 6.9 Hz, 6H). MS ESI m/z calcd for C₃₄H₄₃N₄O₇S [M+H]⁺ 651.27, found 651.39.

### Example 190. Synthesis of tert-butyl ((2R,4S)-1-(4-(benzyloxy)phenyl)-5-((S)-4-isopropyl-2-oxooxazolidin-3-yl)-4-methyl-5-oxopentan-2-yl)carbamate

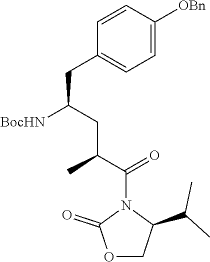 [SMILES: CC(C)[C@H]1COC(=O)N1C(=O)[C@@H](C)C[C@H](Cc1ccc(OCc2ccccc2)cc1)NC(=O)OC(C)(C)C]

To a solution of O-((2S,3S,4S)-5-(4-(benzyloxy)phenyl)-4-((tert-butoxycarbonyl)amino)-1-((S)-4-isopropyl-2-oxooxazolidin-3-yl)-2-methyl-1-oxopentan-3-yl) 1H-imidazole-1-carbothioate (210 g, 0.323 mol, 1.0 eq.) in anhydrous toluene (3 L) was added n-Bu₃SnH (182 g, 0.646 mol, 2.0 eq.) and azodiisobutyronitrile (0.5 g, 3.23 mmol, 0.1 eq.) in sequence. The mixture was refluxed for 1.0 h and then concentrated. The residue was mixed with 500 g silica gel and purified by column chromatography (2 kg silica gel, pure PE to 5:1 PE/EtOAc) to give the title compound as a white foam (141 g, 83% yield). ¹H NMR (400 MHz, CDCl₃) δ 7.36 (ddd, J=24.5, 14.5, 7.1 Hz, 5H), 7.08 (d, J=8.5 Hz, 2H), 6.90 (d, J=8.5 Hz, 2H), 5.04 (d, J=5.1 Hz, 2H), 4.48 (d, J=4.2 Hz, 1H), 4.33 (t, J=8.4 Hz, 1H), 4.22 (d, J=9.7 Hz, 1H), 4.15 (d, J=8.8 Hz, 1H), 3.81 (s, 2H), 2.73 (dd, J=14.1, 5.9 Hz, 1H), 2.61 (dd, J=14.0, 7.2 Hz, 1H), 2.29 (dq, J=13.5, 6.8 Hz, 1H), 2.11-2.00 (m, 1H), 1.60 (dd, J=15.2, 6.2 Hz, 2H), 1.35 (s, 9H), 1.20 (d, J=6.9 Hz, 3H), 0.89 (dd, J=14.0, 6.9 Hz, 6H). MS ESI m/z calcd for C₃₀H₄₁N₂O₆ [M+H]⁺ 525.28, found 525.37.

### Example 191. Synthesis of (2S,4R)-5-(4-(benzyloxy)phenyl)-4-((tert-butoxycarbonyl)amino)-2-methylpentanoic Acid

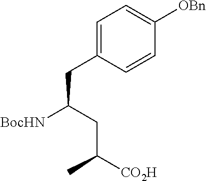 [SMILES: C[C@@H](C[C@H](Cc1ccc(OCc2ccccc2)cc1)NC(=O)OC(C)(C)C)C(=O)O]

To a solution of tert-butyl ((2R,4S)-1-(4-(benzyloxy)phenyl)-5-((S)-4-isopropyl-2-oxooxazolidin-3-yl)-4-methyl-5-oxopentan-2-yl)carbamate (208 g, 0.39 mol, 1.0 equiv) in THE (2.1 L) and water (700 mL) were added LiOH (23.7 g, 0.99 mmol, 2.5 eq.) in H₂O₂(30% aqueous solution, 336 mL, 2.97 mol, 7.6 eq.) at 0° C. After stirring at 0° C. for 3 h, sodium bisulfite solution (1.5 M, 2 L) was added to quench the reaction and 2 N HCl was added dropwise until pH 4 was reached. The reaction mixture was then extracted with EtOAc (3×800 mL). The EtOAc solution was washed with water (500 mL) and brine (500 mL), dried over anhydrous Na2SO4, filtered and concentrated. The residue was mixed with silica gel (400 g) and purified by column chromatography (2 kg silica gel, pure PE to 3:1 PE/EtOAc) to give the title compound as a white solid (158 g, 96% yield). ¹H NMR (400 MHz, CDCl₃) δ 7.46-7.28 (m, 5H), 7.07 (d, J=7.7 Hz, 2H), 6.91 (d, J=7.8 Hz, 2H), 5.04 (s, 2H), 4.52 (d, J=8.5 Hz, 1H), 3.87 (d, J=41.8 Hz, 1H), 2.82-2.43 (m, 3H), 1.85 (t, J=12.2 Hz, 1H), 1.41 (s, 9H), 1.17 (d, J=6.9 Hz, 3H). MS ESI m/z calcd for C₂₄H₃₂NO₅ [M+H]⁺ 414.22, found 414.21.

### Example 192. Synthesis of (2S,4R)-4-((tert-butoxycarbonyl)amino)-5-(4-hydroxyphenyl)-2-methylpentanoic Acid

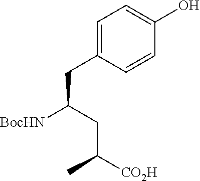 [SMILES: C[C@@H](C[C@H](Cc1ccc(O)cc1)NC(=O)OC(C)(C)C)C(=O)O]

A mixture of (2S,4R)-5-(4-(benzyloxy)phenyl)-4-((tert-butoxycarbonyl)amino)-2-methylpentanoic acid (158 g, 0.38 mol, 1.0 eq.) and Pd/C (10%, 15 g) in methanol (1.5 L) was hydrogenated under 1 atm H₂ pressure for 16 h and then filtered through Celite (filter aid). The filtrate was concentrated to afford the title compound as a white solid (123 g, >100% yield). ¹H NMR (400 MHz, CDCl₃) δ 7.00 (d, J=7.5 Hz, 2H), 6.80 (s, 2H), 4.51 (d, J=9.0 Hz, 1H), 3.88 (s, 1H), 2.66 (dd, J=65.6, 22.6 Hz, 4H), 1.88 (t, J=12.2 Hz, 1H), 1.42 (s, 9H), 1.14 (d, J=6.6 Hz, 3H). MS ESI m/z calcd for C₁₇H₂₆NO₅ [M+H]⁺: 324.17, found 324.16.

### Example 193. Synthesis of (2S,4R)-4-((tert-butoxycarbonyl)amino)-5-(4-hydroxy-3-nitrophenyl)-2-methylpentanoic Acid

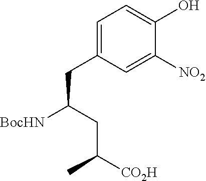 [SMILES: C[C@@H](C[C@H](Cc1ccc(O)c([N+](=O)[O-])c1)NC(=O)OC(C)(C)C)C(=O)O]

To a solution of (2S,4R)-4-((tert-butoxycarbonyl)amino)-5-(4-hydroxyphenyl)-2-methylpentanoic acid (113 g, 0.35 mol, 1.0 eq.) in THE (1.5 L) was added t-BuONO (360 g, 3.5 mol, 10.0 eq.) dropwise and stirred at r.t. for 3 h then mixed with silica gel (300 g) and concentrated, loaded on a column (1.5 kg silica gel) and eluted with pure PE, 5:1 PE/EtOAc and 2:1 PE/EtOAc to give the title compound as a yellow solid (85 g, 61% yield). ¹H NMR (400 MHz, DMSO) δ 12.00 (s, 1H), 10.68 (s, 1H), 7.67 (s, 1H), 7.34 (d, J=8.4 Hz, 1H), 7.03 (d, J=8.4 Hz, 1H), 6.69 (d, J=8.9 Hz, 1H), 3.56 (d, J=3.8 Hz, 1H), 2.67 (dd, J=13.5, 5.1 Hz, 1H), 2.41 (dd, J=13.8, 6.6 Hz, 1H), 1.78-1.65 (m, 1H), 1.27 (s, 9H), 1.18 (s, 1H), 1.05 (d, J=7.1 Hz, 3H). MS ESI m/z calcd for C₁₇H₂₅N₂O₇ [M+H]⁺ 369.15, found 369.14.

### Example 194. Synthesis of (2S,4R)-5-(3-amino-4-hydroxyphenyl)-4-((tert-butoxycarbonyl)amino)-2-methylpentanoic Acid

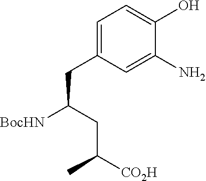 [SMILES: C[C@@H](C[C@H](Cc1ccc(O)c(N)c1)NC(=O)OC(C)(C)C)C(=O)O]

A mixture of (2S,4R)-4-((tert-butoxycarbonyl)amino)-5-(4-hydroxy-3-nitrophenyl)-2-methylpentanoic acid (51.6 g, 0.14 mol, 1.0 eq.) and Pd/C (10 wt %, 5 g) in methanol (500 mL) was hydrogenated (1 atm H₂) at r.t. for 2 h, and then filtered through Celite (filter aid). The filtrate was concentrated to afford the title compound as a brown foam (43.8 g, 93% yield). MS ESI m/z calcd for C₁₇H₂₇N₂O₅ [M+H]⁺ 339.18, found 339.17.

### Example 195. Synthesis of 4-(((benzyloxy)carbonyl)amino)butanoic Acid

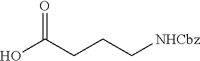 [SMILES: O=C(O)CCCNC(=O)OCc1ccccc1]

To a solution of NaOH (23.3 g, 0.58 mol, 2.0 eq) in water (140 mL) was added 4-aminobutanoic acid (30.0 g, 0.29 mol, 1.0 eq) and THE (60 mL) at −20° C., then CbzCl (54 mL, 0.38 mol, 1.3 eq) in THE (57 mL) was added dropwise. The reaction mixture was stirred at room temperature for 4 h, then concentrated and washed with EtOAc (4×100 mL). Concentrated hydrochloric acid was added to the aqueous solution until pH 3 was reached. The solution was extracted with EA (4×150 mL, 2×100 mL), and the combined organic phase was washed with brine, dried over anhydrous sodium sulfate, filtered, and concentrated to give the title compound as a white solid (48.3 g, 70.3%). ESI m/z: calcd for C₁₂H₁₆NO₄ [M+H]⁺ 238.1, found 238.1.

### Example 196. Synthesis of tert-butyl 4-(((benzyloxy)carbonyl)amino)butanoate

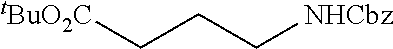 [SMILES: CC(C)(C)OC(=O)CCCNC(=O)OCc1ccccc1]

To a solution of 4-(((benzyloxy)carbonyl)amino)butanoic acid (48.0 g, 0.2 mol, 1.0 eq.) and t-BuOH (58.0 mL, 0.6 mol, 3.0 eq.) in anhydrous dichloromethane (480 mL) were added DCC (50.0 g, 0.24 mol, 1.2 eq.) and DMAP (2.5 g, 0.02 mol, 0.1 eq.) at 0° C., and the mixture then was warmed to room temperature and stirred overnight. The solid was filtered off and the filtrate was concentrated, then diluted with EtOAc (400 mL) and washed with 5% NaHCO₃ solution and brine, dried over anhydrous sodium sulfate, filtered, then concentrated. The residue was purified by SiO₂ column chromatography (PE/EtOAc=5:1) to give the title compound as a colorless oil (32.8 g, 55.1%). ESI m/z: calcd for C₁₆H₂₄NO₄ [M+H]⁺ 294.2, found 294.2.

### Example 197. Synthesis of tert-butyl 4-aminobutanoate

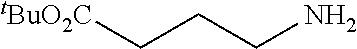 [SMILES: CC(C)(C)OC(=O)CCCN]

To a solution of tert-butyl 4-(((benzyloxy)carbonyl)amino)butanoate (29.0 g, 0.099 mol, 1.0 eq.) in MeOH (100 mL) was added Pd/C (2.9 g, 10% Pd/C, 50% wet) in a hydrogenation bottle. The mixture was shaken under 1 atm H₂ overnight. The reaction mixture was filtered, and the filtrate was concentrated to give the title compound as a colorless oil (13.8 g, 83.7% yield). ESI m/z: calcd for C₈H₁₈NO₂ [M+H]⁺ 160.1, found 160.1.

### Example 198. Synthesis of tert-butyl 2,5,8,11,14,17,20,23,26-nonaoxaoctacosan-28-oate

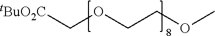

NaH (60%, 24 g, 600 mmol) was added to a solution of octaethylene glycol monomethyl ether (115 g, 300 mmol) in THE (3.0 L). After stirring at r.t. for 1 h, tert-butyl 2-bromoacetate (146 g, 750 mmol) was added to the mixture, and stirred at r.t. for 1 h. The mixture was then diluted with dichloromethane (4 L) and poured onto ice water (2 kg). The organic phase was separated and aqueous phase was extracted with dichloromethane (1 L). The combined organic phases were washed with water, dried over anhydrous Na₂SO₄. Purification by column chromatography (20% EtOAc/PE, then pure DCM to 5% MeOH/DCM) yielded the title compound as a yellow oil (108 g, 72% yield).

### Example 199. Synthesis of 2,5,8,11,14,17,20,23,26-nonaoxaoctacosan-28-oic Acid

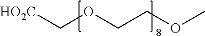

Tert-butyl 2,5,8,11,14,17,20,23,26-nonaoxaoctacosan-28-oate (210 g, 422 mmol) was dissolved in dichloromethane (400 mL) anhydrous formic acid (1 L). The resulting solution was stirred at r.t. overnight. All volatiles were removed under vacuum, which afforded the title compound as a yellow oil (200 g, >100% yield).

### Example 200. Synthesis of 2,5,8,11,14,17,20,23,26-nonaoxaoctacosan-28-oyl chloride

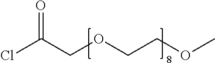

To the solution of 2,5,8,11,14,17,20,23,26-nonaoxaoctacosan-28-oic acid (198 g, 422 mmol) dissolved in dichloromethane (2.6 L), (COCl)₂ (275 mL) and DMF (0.5 mL) were added at r.t. The resulting solution was stirred at r.t. for 3 h. All volatiles were removed under vacuum to yield the title compound as a yellow oil (210 g, >100% yield).

### Example 201. Synthesis of (S)-34-(((benzyloxy)carbonyl)amino)-28-oxo-2,5,8,11,14,17,20,23,26-nonaoxa-29-azapentatriacontan-35-oic Acid

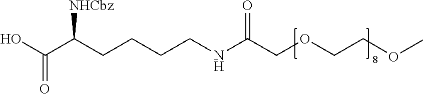

Z-L-Lys-OH (236 g, 844 mmol), Na₂CO₃ (89.5 g, 844 mmol) and NaOH (33.8 g, 844 mmol) were dissolved in water (1.6 L). The mixture was cooled under 0° C. using ice salt bath, to which a solution of 2,5,8,11,14,17,20,23,26-nonaoxaoctacosan-28-oyl chloride (210 g, 422 mmol) in THE (160 mL) was added. The resulting mixture was stirred at r.t. for 1 h, and then diluted with EtOAc (1 L). The aqueous layer was separated, to which concentrated HCl was added under ice cooling until pH 3 was reached. After extraction with DCM, the organic layer was washed with brine, dried over Na₂SO₄ and concentrated to give the title compound as a yellow oil (290 g, 97% yield).

### Example 202. Synthesis of (S)-perfluorophenyl 34-(((benzyloxy)carbonyl)amino)-28-oxo-2,5,8,11,14,17,20,23,26-nonaoxa-29-azapentatriacontan-35-oate

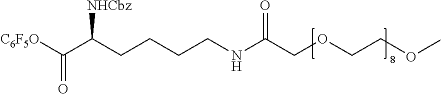

To a solution of (S)-34-(((benzyloxy)carbonyl)amino)-28-oxo-2,5,8,11,14,17,20,23,26-nonaoxa-29-azapentatriacontan-35-oic acid (183 g, 260 mmol) in dichloromethane (2 L) was added pentafluorophenol (95.4 g, 520 mmol) and DIC (131 g, 1.04 mol). The reaction was stirred at r.t. for 1 h, and then concentrated to give crude the title product (430 g).

### Example 203. Synthesis of (S)-tert-butyl 34-(((benzyloxy)carbonyl)amino)-28,35-dioxo-2,5,8,11,14,17,20,23,26-nonaoxa-29,36-diazatetracontan-40-oate

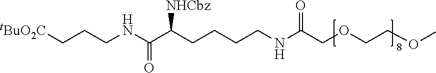

To a solution of tert-butyl 4-aminobutanoate (62.0 g, 390 mmol) in DMF (1.5 L) was added DIPEA (134 g, 1.04 mol) at 0° C. (S)-perfluorophenyl 34-(((benzyloxy)carbonyl)-amino)-28-oxo-2,5,8,11,14,17,20,23,26-nonaoxa-29-azapentatriacontan-35-oate (430 g, crude) was then added at 10-20° C. and the resulting mixture was stirred at r.t. for 1 h. DMF was removed under vacuum and the residue was diluted with dichloromethane, washed with water. The aqueous phase was back-extracted with dichloromethane. The combined organic phase was washed with 0.2 N HCl and brine, dried over anhydrous Na₂SO₄, filtered and concentrated. Column chromatography (25% EtOAc/PE to pure EtOAc, then 0 to 5% MeOH/DCM) gave the title compound as a yellow oil (180 g, 82% yield).

### Example 204. Synthesis of (S)-tert-butyl 34-amino-28,35-dioxo-2,5,8,11,14,17,20,23,26-nonaoxa-29,36-diazatetracontan-40-oate

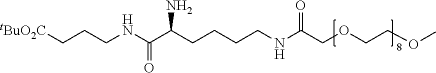

To a solution of (S)-tert-butyl 34-(((benzyloxy)carbonyl)amino)-28,35-dioxo-2,5,8,11,14,17,20,23,26-nonaoxa-29,36-diazatetracontan-40-oate (78.0 g, 92.3 mmol, 1.0 eq.) in MeOH (500 mL) was added Pd/C (13 g, 10% Pd/C, 50% wet). The mixture was hydrogenated under 1 atm H₂ at r.t. overnight, then filtered and concentrated. The residue was purified by column chromatography (0 to 20% MeOH/DCM) to give the title compound as a greenish yellow oil (70.2 g, 92% yield).

### Example 205. Synthesis of 11-(benzyloxy)-11-oxoundecanoic Acid

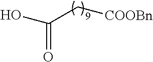

To a solution of undecanedioic acid (1.73 g, 8 mmol) in DMF (30 mL) were added K₂CO₃(1.1 g, 8 mmol) and BnBr (1.36 g, 8 mmol). The mixture was stirred at r.t. overnight, then concentrated and purified by column chromatography (PE/EtOAc) to afford the title compound (1.1 g, 45% yield). ESI m/z: calcd for C₁₈H₂₇O₄ [M+H]⁺: 307.18, found 307.15.

### Example 206. Synthesis of 3-(2-(2-(dibenzylamino)ethoxy)ethoxy)propanoic Acid

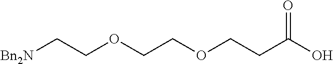

To a solution of tert-butyl 3-(2-(2-(dibenzylamino)ethoxy)ethoxy)propanoate (2.00 g, 4.84 mmol) in DCM (5 mL) was added HCO₂H (5 mL). The reaction was stirred at room temperature overnight, then concentrated to dryness and co-evaporated twice with DCM, and the residue was placed on a pump to give the title compound (1.72 g, ˜100% yield). ESI m/z calcd for C₂₁H₂₇NO₄ [M+H]⁺: 358.19, found 358.19.

### Example 207. Synthesis of tert-butyl 2-benzyl-11-oxo-1-phenyl-5,8,15,18-tetraoxa-2,12-diazahenicosan-21-oate

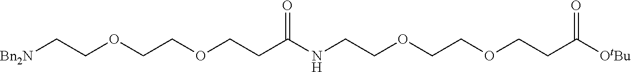

To a solution of 3-(2-(2-(dibenzylamino)ethoxy)ethoxy)propanoic acid (1.12 g, 4.83 mmol) and tert-butyl 3-(2-(2-aminoethoxy)ethoxy)propanoate (1.72 g, 4.83 mmol) in DCM (30 mL) were added HATU (1.83 g, 4.83 mmol) and TEA (0.68 mL, 4.83 mmol) at 0° C. The reaction was warmed to r.t. and stirred for 1 h, then diluted with 50 mL DCM and poured into a separatory funnel containing 50 mL of water. The organic phase was separated, and washed with brine (50 mL), dried over anhydrous Na₂SO₄, filtered and concentrated. The residue was purified by column chromatography (MeOH/DCM) to afford the title compound (2.21 g, 80% yield). ESI m/z calcd for C₃₂H₄₈N₂O₇[M+H]⁺: 573.35, found 573.35.

### Example 208. Synthesis of tert-butyl 1-amino-9-oxo-3,6,13,16-tetraoxa-10-azanonadecan-19-oate

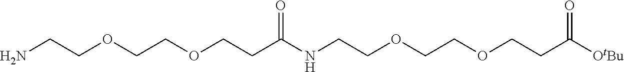 [SMILES: CC(C)(C)OC(=O)CCOCCOCCNC(=O)CCOCCOCCN]

To a solution of tert-butyl 2-benzyl-11-oxo-1-phenyl-5,8,15,18-tetraoxa-2,12-diazahenicosan-21-oate (2.21 g, 3.86 mmol) in MeOH (20 mL) was added Pd/C (10 wt %, 0.2 g) in a hydrogenation bottle. The mixture was stirred under 1 atm H₂ overnight, filtered through Celite (filter aid), and the filtrate was concentrated to afford the title compound (1.5 g, ˜100% yield). ESI m/z calcd for C₁₈H₃₆N₂O₇ [M+H]⁺: 393.25, found 393.25.

### Example 209. Synthesis of 31-benzyl 1-tert-butyl 11,21-dioxo-4,7,14,17-tetraoxa-10,20-diazahentriacontane-1,31-dioate

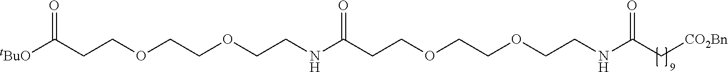

To a solution of tert-butyl 1-amino-9-oxo-3,6,13,16-tetraoxa-10-azanonadecan-19-oate (1.50 g, 3.86 mmol) and 11-(benzyloxy)-11-oxoundecanoic acid (1.10 g, 3.6 mmol) in DCM (50 mL) were added HATU (1.48 g, 3.9 mmol) and TEA (0.55 mL, 3.9 mmol) at 0° C. The reaction mixture was stirred at r.t. for 1 h, then diluted with 50 mL DCM and poured into a separatory funnel containing 50 mL of water. The organic phase was separated, washed with brine (50 mL), dried over anhydrous Na₂SO₄, filtered and concentrated. The residue was purified by column chromatography (MeOH/DCM) to afford the title compound (1.50 g, 61% yield). ESI m/z calcd for C₃₆H₆₁N₂O₁₀ [M+H]⁺: 681.42, found 681.42.

### Example 210. Synthesis of 3,13,23-trioxo-1-phenyl-2,17,20,27,30-pentaoxa-14,24-diazatritriacontan-33-oic Acid

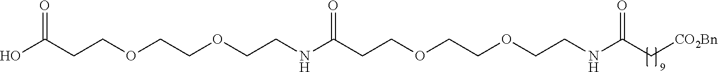

To a solution of 31-benzyl 1-tert-butyl 11,21-dioxo-4,7,14,17-tetraoxa-10,20-diazahentriacontane-1,31-dioate (1.50 g, 2.2 mmol) in DCM (1 mL) was added TFA (3 mL). The reaction was stirred at room temperature for 1 h, then concentrated to dryness and co-evaporated twice with DCM, and the residue was placed on a pump to give the title compound (0.09 g, 2.2 mmol, crude product). ESI m/z: calcd for C₃₂H₅₃N₂O₁₀ [M+H]⁺: 625.36, found 625.35.

### Example 211. Synthesis of (S)-39-(((benzyloxy)carbonyl)amino)-3,13,23,33-tetraoxo-1-phenyl-2,17,20,27,30-pentaoxa-14,24,34-triazatetracontan-40-oic Acid

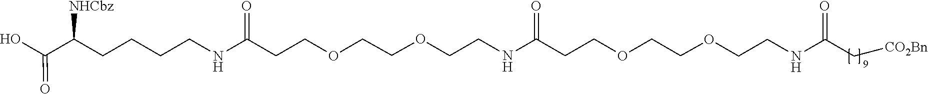

To a solution of3,13,23-trioxo-1-phenyl-2,17,20,27,30-pentaoxa-14,24-diazatritriacontan-33-oic acid (1.50 g, 2.20 mmol) and Z-Lys-OH (0.62 g, 2.20 mmol) in DCM (50 mL) were added HATU (0.84 g, 2.20 mmol) and TEA (0.31 mL, 2.20 mmol) at 0° C. The reaction mixture was stirred at r.t. for 1h, then diluted with 50 mL DCM and poured into a separatory funnel containing 100 mL of water. The organic phase was separated, and washed with brine (100 mL), dried over anhydrous Na₂SO₄, filtered and concentrated. The residue was purified by column chromatography (MeOH/DCM) to afford the title compound (1.00 g, 53% yield). ESI m/z calcd for C₄₆H₇₁N₄O₁₃ [M+H]⁺: 887.49, found 887.50.

### Example 212. Synthesis of (S)-benzyl 5-((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-3,11,21,31-tetraoxo-1-phenyl-2,14,17,24,27-pentaoxa-4,10,20,30-tetraazahentetracontan-41-oate

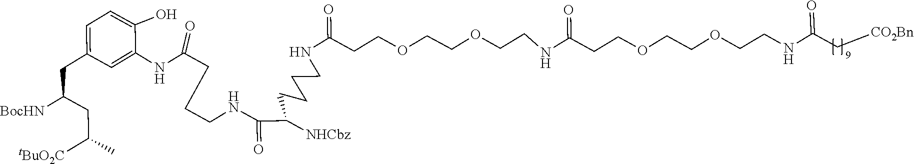

To a solution of (S)-39-(((benzyloxy)carbonyl)amino)-3,13,23,33-tetraoxo-1-phenyl-2,17,20,27,30-pentaoxa-14,24,34-triazatetracontan-40-oic acid (0.50 g, 0.56 mmol) in DMF (5 mL) was added HATU (0.21 g, 0.56 mmol) and the reaction was stirred at room temperature for 30 min. After that, a solution of (2S,4R)-tert-butyl 5-(3-(4-aminobutanamido)-4-hydroxyphenyl)-4-((tert-butoxycarbonyl)amino)-2-methylpentanoate (0.27 g, 0.56 mmol) in DMF (5 mL) and TEA (85 μL, 0.6 mmol) were added in sequence at 0° C., and the reaction was stirred for 1 h. The reaction mixture was poured into a separatory funnel containing 100 mL of water and extracted with 50 mL of EtOAc twice. The organic phase was washed once with 100 mL of brine, dried over anhydrous Na2SO4, filtered and concentrated. The residue was purified by column chromatography (MeOH/DCM) to afford the title compound (0.40 g, 55% yield). ESI m/z: calcd for C₇₁H₁₁₀N₇O₁₈ [M+H]⁺: 1348.78, found 1348.78.

### Example 213. Synthesis of (S)-benzyl 5-((5-((2R,4S)-5-(tert-butoxy)-2-((tert-butoxycarbonyl)amino)-4-methyl-5-oxopentyl)-2-hydroxyphenyl)carbamoyl)-3,11,21,31-tetraoxo-1-phenyl-2,14,17,24,27-pentaoxa-4,10,20,30-tetraazahentetracontan-41-oate

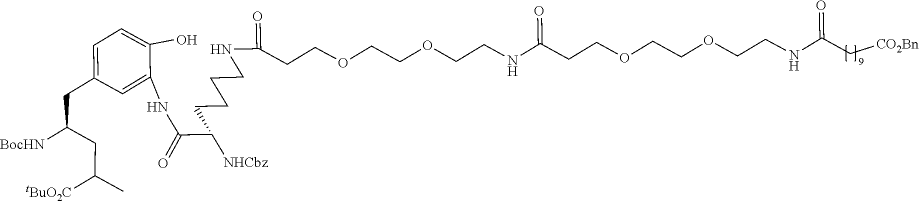

To a solution of (S)-39-(((benzyloxy)carbonyl)amino)-3,13,23,33-tetraoxo-1-phenyl-2,17,20,27,30-pentaoxa-14,24,34-triazatetracontan-40-oic acid (0.50 g, 0.56 mmol) in DMF (5 mL) was added HATU (0.21 g, 0.56 mmol) and the reaction was stirred at room temperature for 30 min. After that, a solution of (2S,4R)-tert-butyl 5-(3-amino-4-hydroxyphenyl)-4-((tert-butoxycarbonyl)amino)-2-methylpentanoate (0.22 g, 0.56 mmol) in DMF (5 mL) and TEA (85 μL, 0.60 mmol) were added at 0° C. After stirring for 1 h, the reaction mixture was poured into a separatory funnel containing 100 mL of water and extracted with 50 mL of EtOAc twice. The organic phase was separated and washed with 100 mL of brine, dried over anhydrous Na₂SO₄, filtered and concentrated. The residue was purified by column chromatography (MeOH/DCM) to afford the title compound (0.20 g, 26% yield). ESI m/z: calcd for C₆₇H₁₀₃N₆O₁₇ [M+H]⁺: 1263.73, found 1263.73.

### Example 214. Synthesis of di-tert-butyl 3,3′-((oxybis(ethane-2,1-diyl))bis(oxy))dipropanoate

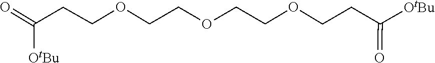 [SMILES: CC(C)(C)OC(=O)CCOCCOCCOCCC(=O)OC(C)(C)C]

To a solution of diethylene glycol (20 g, 0.188 mol) in THE (200 mL) was added Na (0.43 g, 0.018 mol). After stirring at r.t. for 1 h, tert-butyl acrylate (48 g, 0.376 mol) was added and the reaction mixture was stirred at r.t. for 2 days. The reaction was concentrated under vacuum and purified by column chromatography to afford the title compound (34 g, 50% yield). ESI m/z calcd for C₁₈H₃₅O₇[M+H]⁺: 363.23, found 363.23.

### Example 215. Synthesis of 3,3′-((oxybis(ethane-2,1-diyl))bis(oxy))dipropanoic Acid

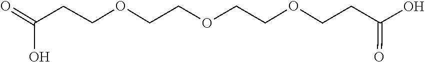 [SMILES: O=C(O)CCOCCOCCOCCC(=O)O]

Di-tert-butyl 3,3′-((oxybis(ethane-2,1-diyl))bis(oxy))dipropanoate (34 g, 0.093 mol) was dissolved in formic acid (100 mL) at room temperature and stirred overnight. The reaction was concentrated under vacuum to afford the title compound. ESI m/z calcd for C₁₀H₁₉O₇[M+H]⁺: 251.11, found 251.11.

### Example 216. Synthesis of2,2-dimethyl-4,14,24-trioxo-3,7,10,17,20,27,30,33-octaoxa-13,23-diazahexatriacontan-36-oic Acid

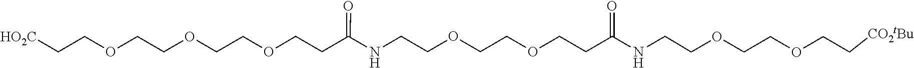 [SMILES: CC(C)(C)OC(=O)CCOCCOCCNC(=O)CCOCCOCCNC(=O)CCOCCOCCOCCC(=O)O]

To a solution of tert-butyl 1-amino-9-oxo-3,6,13,16-tetraoxa-10-azanonadecan-19-oate (1.50 g, 3.82 mmol) and 3,3′-((oxybis(ethane-2,1-diyl))bis(oxy))dipropanoic acid (1.90 g, 7.64 mmol) in DMF (10 mL) were added HATU (1.45 g, 3.82 mmol) and DIPEA (0.66 mL, 3.82 mmol) at 0° C. The reaction mixture was warmed to r.t. and stirred for 1 h, then diluted with DCM (80 mL), washed with water (10 mL), dried over sodium sulfate, filtered, concentrated and purified by silica gel column chromatography to afford the title compound as a colorless liquid (1.75 g, 75% yield). ESI m/z calcd for C₂₈H₅₃N₂O₁₃ [M+H]⁺: 625.35, found 625.35.

### Example 217. Synthesis of 1-tert-butyl 33-(2,5-dioxopyrrolidin-1-yl) 11,21-dioxo-4,7,14,17,24,27,30-heptaoxa-10,20-diazatritriacontane-1,33-dioate

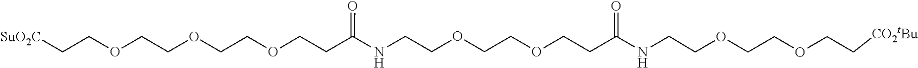

To a solution of2,2-dimethyl-4,14,24-trioxo-3,7,10,17,20,27,30,33-octaoxa-13,23-diazahexatriacontan-36-oic acid (1.75 g, 2.8 mmol) in DCM (20 mL) were added EDCI (1.07 g, 5.6 mmol) and NHS (0.64 g, 5.6 mmol) at 0° C. The reaction was warmed to room temperature and stirred overnight, then diluted with DCM (80 mL), washed with water (10 mL), dried over sodium sulfate, filtered and concentrated under vacuum to afford the title compound (2.00 g, ˜100% yield). ESI m/z calcd for C₃₂H₅₆N₃O₁₅ [M+H]⁺: 722.36, found 722.36.

### Example 218. Synthesis of (S)-42-(((benzyloxy)carbonyl)amino)-2,2-dimethyl-4,14,24,36-tetraoxo-3,7,10,17,20,27,30,33-octaoxa-13,23,37-triazatritetracontan-43-oic Acid

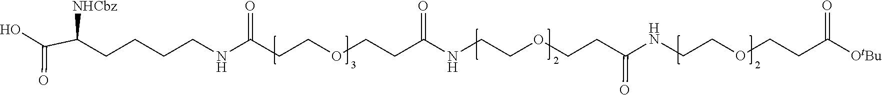

To a solution of N-α-Cbz-L-lysine (1.17 g, 4.2 mmol) in water (10 mL) was added sodium bicarbonate (0.47 g, 5.6 mmol), and the reaction mixture was cooled to 5° C., and 1-tert-butyl 33-(2,5-dioxopyrrolidin-1-yl) 11,21-dioxo-4,7,14,17,24,27,30-heptaoxa-10,20-diazatritriacontane-1,33-dioate (2.00 g, 2.8 mmol) dissolved in 1,4-Dioxane (10 mL) was added. The reaction was warmed to r.t. and stirred for 1 h, then acidified to pH 3 by addition of 1 N HCl, extracted with DCM (50 mL×3). The organic extracts were washed with water (20 mL), dried over sodium sulfate, filtered and concentrated to afford the title product (2.3 g, 92% yield). ESI m/z calcd for C₄₂H₇₁N₄O₁₆ [M+H]⁺: 887.48, found 887.48.

### Example 219. Synthesis of (S)-tert-butyl 5-((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-3,11,23,33-tetraoxo-1-phenyl-2,14,17,20,27,30,37,40-octaoxa-4,10,24,34-tetraazatritetracontan-43-oate

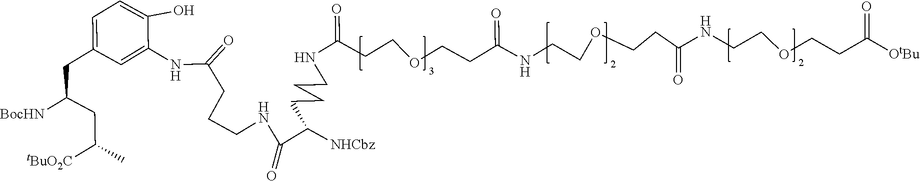

To a solution of (2S,4R)-tert-butyl 5-(3-(4-aminobutanamido)-4-hydroxyphenyl)-4-((tert-butoxycarbonyl)amino)-2-methylpentanoate (1.87 g, 3.9 mmol) and (S)-42-(((benzyloxy)-carbonyl)amino)-2,2-dimethyl-4,14,24,36-tetraoxo-3,7,10,17,20,27,30,33-octaoxa-13,23,37-triazatritetracontan-43-oic acid (2.3 g, 2.59 mmol) in dichloromethane (30 mL) were added HATU (0.98 g, 2.59 mmol) and DIPEA (450 μL, 2.59 mmol) at 0° C. The reaction mixture was warmed to r.t. and stirred for 1 h, then concentrated under vacuum and purified by silica gel column chromatography to afford the title compound (2.4 g, 70% yield). ESI m/z calcd for C₆₇H₁₁₀N₇O₂₁ [M+H]⁺: 1348.77, found 1348.77.

### Example 220. Synthesis of (S)-43-benzyl 1-tert-butyl 7-(((benzyloxy)carbonyl)amino)-6,13,23,33-tetraoxo-16,19,26,29-tetraoxa-5,12,22,32-tetraazatritetracontane-1,43-dioate

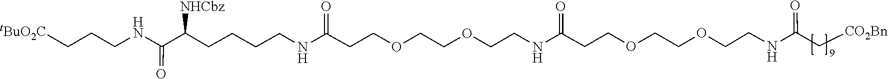

(S)-39-(((benzyloxy)carbonyl)amino)-3,13,23,33-tetraoxo-1-phenyl-2,17,20,27,30-pentaoxa-14,24,34-triazatetracontan-40-oic acid (200 mg, 0.225 mmol) was dissolved in DMF (5 mL) and cooled to 0° C., tert-butyl 4-Aminobutanoate (71.8 mg, 0.45 mmol) and EDC (86.2 mg, 0.45 mmol) were added in sequence. The reaction was warmed to r.t. and stirred overnight, poured into ice-water, and extraction with DCM (3×10 mL). The combined organic phase was washed with water (5 mL), brine (5 mL), dried over anhydrous Na₂SO₄, filtered and concentrated to give the title compound (231 mg, 100% yield). ESI m/z calcd for C₅₄H₈₆N₅O₁₄ [M+H]⁺:1028.61, found: 1028.61.

### Example 221. Synthesis of (S)-43-benzyl 1-(2-((S)-39-(((benzyloxy)carbonyl)amino)-3,13,23,33,40-pentaoxo-1-phenyl-2,17,20,27,30-pentaoxa-14,24,34,41-tetraazapentatetracontanamido)-4-((2R,4S)-5-(tert-butoxy)-2-((tert-butoxycarbonyl)amino)-4-methyl-5-oxopentyl)phenyl) 7-(((benzyloxy)carbonyl)amino)-6,13,23,33-tetraoxo-16,19,26,29-tetranoxa-5, 12,22,32-tetraazatritetracontane-1,43-dioate

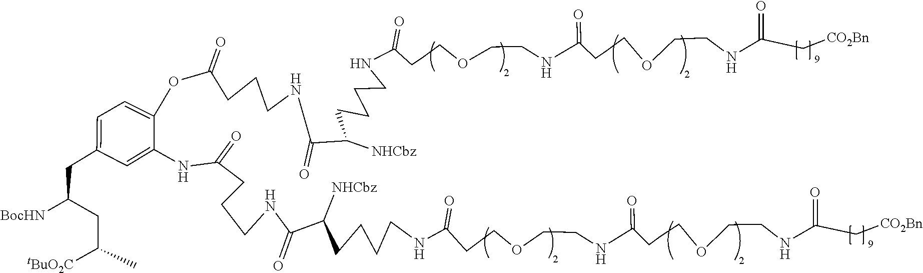

(S)-43-Benzyl 1-tert-butyl 7-(((benzyloxy)carbonyl)amino)-6,13,23,33-tetraoxo-16,19,26,29-tetraoxa-5,12,22,32-tetraazatritetracontane-1,43-dioate (231 mg, 0.225 mmol) was dissolved in DCM (3 mL) and treated with TFA (3 mL) at r.t. for 1 h. The reaction was concentrated and re-dissolved in DMF (5 mL) and cooled to 0° C., (2S,4R)-tert-butyl 5-(3-amino-4-hydroxyphenyl)-4-((tert-butoxycarbonyl)amino)-2-methylpentanoate (44 mg, 0.112 mmol), HATU (86 mg, 0.225 mmol) and DIPEA (39 μL, 0.225 mmol) were added in sequence. The reaction was warmed to r.t. and stirred overnight, poured into ice-water, and extraction with DCM (3×10 mL). The combined organic phase was washed with 1 N HCl (5 mL), water (5 mL), brine (5 mL), dried over anhydrous Na₂SO₄, filtered and concentrated, purified by silica gel column chromatography (0-5% MeOH/DCm) to give a white foam (209 mg, 81% yield). ESI m/z calcd for C₁₂₁H₁₈₅N₁₂O₃₁ [M+H]⁺: 2302.32, found: 2302.80.

### Example 222. Synthesis of (S)-7-amino-1-((2-(((R)-7-amino-42-carboxy-6,13,23,33-tetraoxo-16,19,26,29-tetraoxa-5,12,22,32-tetraazadotetracontan-1-oyl)oxy)-5-((2R,4S)-5-(tert-butoxy)-2-((tert-butoxycarbonyl)amino)-4-methyl-5-oxopentyl)phenyl)amino)-1,6,13,23,33-pentaoxo-16,19,26,29-tetraoxa-5,12,22,32-tetraazatritetracontan-43-oic Acid

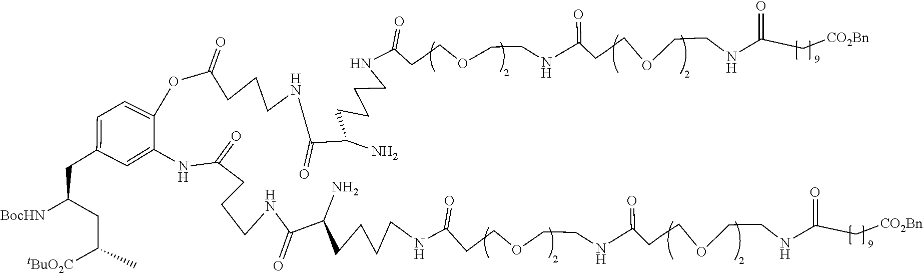

(S)-43-Benzyl 1-(2-((S)-39-(((benzyloxy)carbonyl)amino)-3,13,23,33,40-pentaoxo-1-phenyl-2,17,20,27,30-pentaoxa-14,24,34,41-tetraazapentatetracontanamido)-4-((2R,4S)-5-(tert-butoxy)-2-((tert-butoxycarbonyl)amino)-4-methyl-5-oxopentyl)phenyl) 7-(((benzyloxy)-carbonyl)amino)-6,13,23,33-tetraoxo-16,19,26,29-tetraoxa-5,12,22,32-tetraazatritetracontane-1,43-dioate (206 mg, 0.089 mmol) was dissolved in MeOH (5 mL) and mixed Pd/C (10 wt %, 20 mg), hydrogenated under 1 atm H₂ pressure overnight. The mixture was then filtered through Celite (filter aid), and the filtrate was concentrated to afford the title compound (166 mg, 100% yield). ESI m/z calcd for C₉₁H₁₆₁N₁₂O₂₇ [M+H]⁺: 1854.15, found 1854.80.

### Example 223. Synthesis of 1,1′-((8R,27S)-36-((2R,4S)-5-(tert-butoxy)-2-((tert-butoxycarbonyl)amino)-4-methyl-5-oxopentyl)-17,18-bis(2-(2,5-dioxo-2,5-dihydro-1H-pyrrol-1-yl)acetyl)-2,7,10,15,20,25,28,33-octaoxo-3,4,5,6,7,8,9,10,11,12,13,14,15,16,17,18,19,20,21,22,23,24,25,26,27,28,29,30,31,32,33,34-dotriacontahydro-2H-benzo[b][1,4,9,12,17,20,21,24,29,32]oxanonaazacyclohexatriacontine-8,27-diyl)bis(6,16,26-trioxo-9,12,19,22-tetraoxa-5,15,25-triazahexatriacontan-36-oic acid)

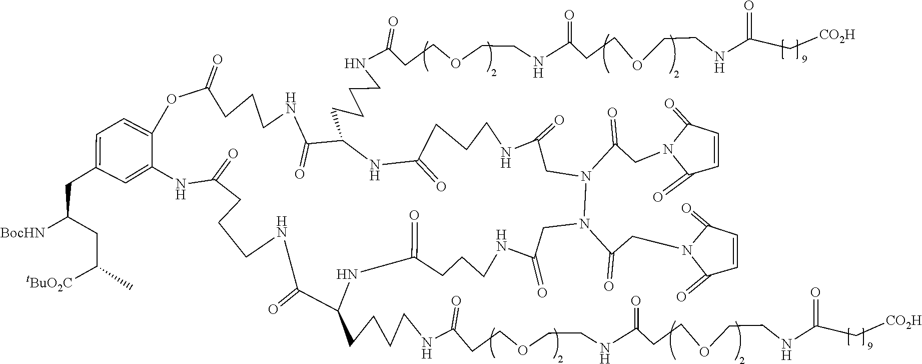

To a solution of (S)-7-amino-1-((2-(((R)-7-amino-42-carboxy-6,13,23,33-tetraoxo-16,19,26,29-tetraoxa-5,12,22,32-tetraazadotetracontan-1-oyl)oxy)-5-((2R,4S)-5-(tert-butoxy)-2-((tert-butoxycarbonyl)amino)-4-methyl-5-oxopentyl)phenyl)amino)-1,6,13,23,33-pentaoxo-16,19,26,29-tetraoxa-5,12,22,32-tetraazatritetracontan-43-oic acid (165 mg, 0.089 mmol) in ethanol (10 mL) were added bis(2,5-dioxopyrrolidin-1-yl) 4,4′-((2,2′-(1,2-bis(2-(2,5-dioxo-2,5-dihydro-1H-pyrrol-1-yl)acetyl)hydrazine-1,2-diyl)bis(acetyl))bis(azanediyl))dibutanoate (70 mg, 0.089 mmol) and phosphate buffer (0.5M, pH 7.5, 3 mL) at 0° C. The reaction was stirred at R.T. overnight and then concentrated and purified by silica gel column chromatography (0-6% MeOH/DCM) to give the title compound 666 (130 mg, 62% yield). ESI m/z calcd for C₁₁₅H₁₈₅N₁₈O₃₇ [M+H]⁺: 2410.31, found: 2410.60.

### Example 224. Synthesis of 1,1′(2-8R,27S)-36-((2R,4S)-2-amino-4-carboxypentyl)-17,18-bis(2-(2,5-dioxo-2,5-dihydro-1H-pyrrol-1-yl)acetyl)-2,7,10,15,20,25,28,33-octaoxo-3,4,5,6,7,8,9,10,11,12,13,14,15,16,17,18,19,20,21,22,23,24,25,26,27,28,29,30,31,32,33,34-dotriacontahydro-2H-benzo[b][1,4,9,12,17,20,21,24,29,32]oxanonaazacyclohexatriacontine-8,27-diyl)bis(6,16,26-trioxo-9,12,19,22-tetraoxa-5,15,25-triazahexatriacontan-36-oic Acid)

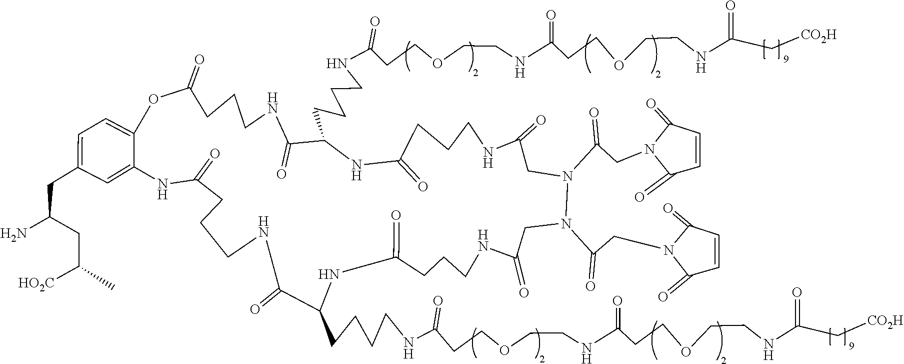

1,1′-((8R,27S)-36-((2R,4S)-5-(tert-butoxy)-2-((tert-butoxycarbonyl)amino)-4-methyl-5-oxopentyl)-17,18-bis(2-(2,5-dioxo-2,5-dihydro-1H-pyrrol-1-yl)acetyl)-2,7,10,15,20,25,28,33-octaoxo-3,4,5,6,7,8,9,10,11,12,13,14,15,16,17,18,19,20,21,22,23,24,25,26,27,28,29,30,31,32,33,34-dotriacontahydro-2H-benzo[b][1,4,9,12,17,20,21,24,29,32]oxanonaazacyclohexa-triacontine-8,27-diyl)bis(6,16,26-trioxo-9,12,19,22-tetraoxa-5,15,25-triazahexatriacontan-36-oic acid) (128 mg, 0.053 mmol) was dissolved in DCM (3 mL) and treated with TFA (3 mL) at r.t. for 2 h. The reaction was concentrated and co-evaporated with DCM for three times to give the title compound (120 mg, 100% yield). ESI m/z calcd for C₁₀₆H₁₆₉N₁₈O₃₅ [M+H]⁺: 2254.19, found: 2254.30.

### Example 225. Synthesis of 1,1′-((8R,27S)-36-((2R,4S)-2-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-4-carboxypentyl)-17,18-bis(2-(2,5-dioxo-2,5-dihydro-1H-pyrrol-1-yl)acetyl)-2,7,10,15,20,25,28,33-octaoxo-3,4,5,6,7,8,9,10,11,12,13,14,15,16,17,18,19,20,21,22,23,24,25,26,27,28,29,30,31,32,33,34-dotriacontahydro-2H-benzo[b][1,4,9,12,17,20,21,24,29,32]-oxanonaazacyclohexatriacontine-8,27-diyl)bis(6,16,26-trioxo-9,12,19,22-tetraoxa-5,15,25-triazahexatriacontan-36-oic Acid) (B-01)

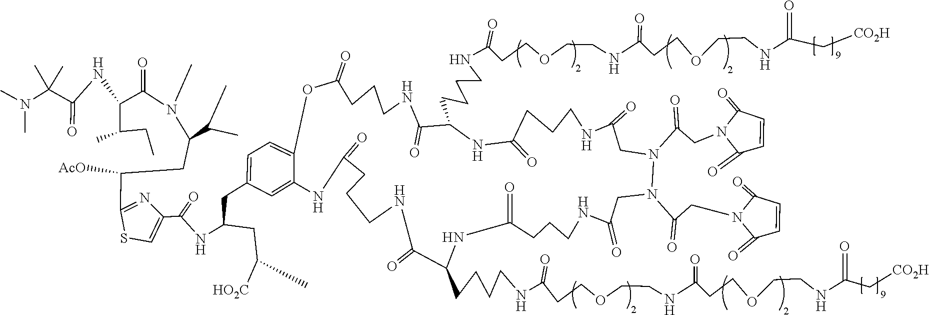

1,1′-((8R,27S)-36-((2R,4S)-2-amino-4-carboxypentyl)-17,18-bis(2-(2,5-dioxo-2,5-dihydro-1H-pyrrol-1-yl)acetyl)-2,7,10,15,20,25,28,33-octaoxo-3,4,5,6,7,8,9,10,11,12,13,14,15,16,17,18, 19,20,21,22,23,24,25,26,27,28,29,30,31,32,33,34-dotriacontahydro-2H-benzo[b][1,4,9,12,17,20,21,24,29,32]oxanonaazacyclohexatriacontine-8,27-diyl)bis(6,16,26-trioxo-9,12,19,22-tetraoxa-5,15,25-triazahexatriacontan-36-oic acid) (120 mg, 0.053 mmol) and compound 41a (36.6 mg, 0.053 mmol) were dissolved in DMA (5 mL) and cooled to 0° C. DIPEA (18 μL, 0.106 mmol) was added and the reaction was warmed to r.t. and stirred for 1 h. After the reaction mixture was concentrated, the residue was purified by prep-HPLC (C₁₈, 10-90% acetonitrile/water) to give the title compound (B-1) (70 mg, 49% yield). ESI m/z calcd for C₁₃₁H₂₀₉N₂₂O₄₀S [M+H]⁺: 2762.46, found: 2762.85.

### Example 226. Synthesis of (7S,10R,11S,14S)-di-tert-butyl 10,11-bis(((benzyloxy)-carbonyl)amino)-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioate

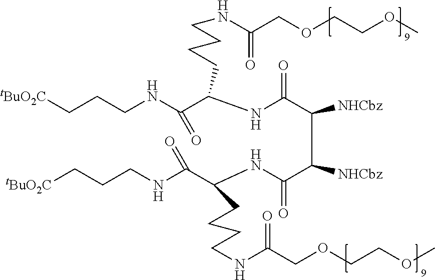

A mixture of (S)-tert-butyl 37-(((benzyloxy)carbonyl)amino)-31,38-dioxo-2,5,8,11,14,17,20,23,26,29-decaoxa-32,39-diazatritetracontan-43-oate (5.98 g, 6.73 mmol) and Pd/C (10 wt %, 0.6 g) in methanol (30 mL) was hydrogenated under 1 atm H₂ pressure overnight and then filtered through Celite (filter aid). The filtrate was concentrated and re-dissolved in THF (60 mL), (2R,3S)-2,3-bis(((benzyloxy)carbonyl)amino)succinic acid (1.01 g, 2.42 mmol) and HOBt (817 mg, 6.05 mmol) were added at 0° C. DCC (1.25 g, 6.05 mmol) and DIPEA (2.1 mL, 12.10 mmol) were added in sequence. The reaction was stirred at r.t. overnight, then diluted with EtOAc (400 mL), and washed with 0.1N HCl, saturated sodium bicarbonate and brine, dried over anhydrous Na2SO₄, filtered, concentrated and purified by SiO₂ column chromatography (24:1 DCM/MeOH) to give the title compound (5.65 g, 49% yield). MS ESI m/z calcd for C₉₀H₁₅₄N₈O₃₄ [M+H]⁺ 1892.06, found 1892.60.

### Example 227. Synthesis of (7S,10R,11S,14S)-di-tert-butyl 10,11-diamino-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioate

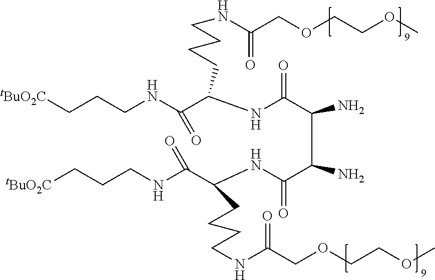

A mixture of (7S,10R,11S,14S)-di-tert-butyl 10,11-bis(((benzyloxy)-carbonyl)amino)-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioate (3.71 g, 1.96 mmol) and Pd/C (10 wt %, 0.40 g) in methanol (50 mL) was hydrogenated under 1 atm H₂ pressure overnight and then filtered through Celite (filter aid). The filtrate was concentrated to afford the title compound (3.18 g, 100% yield). MS ESI m/z calcd for C₇₄H₁₄₂N₈O₃₀ [M+H]⁺ 1623.98, found 1624.50.

### Example 228. Synthesis of (7S,10R,11S,14S)-10,11-bis(4-(2,5-dioxo-2,5-dihydro-1H-pyrrol-1-yl)butanamido)-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioic Acid

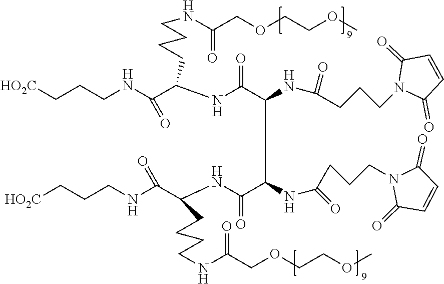

To a solution of (75,10R,11S,14S)-di-tert-butyl 10,11-diamino-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioate (315 mg, 0.194 mmol) in DMA (10 mL) were added EDC (150 mg, 0.785 mmol) and 4-maleido-butanoic acid (72 mg, 0.57 mmol). The mixture was stirred at room temperature for 12 h, concentrated and purified by SiO₂ column chromatography (1:4 MeOH/DCM) to give an oil (329 mg, 87% yield), which was dissolved in dichloromethane (25 mL) and treated with TFA (5 mL) at r.t. for 1h, and then concentrated to afford the title compound (309 mg, 99% yield). MS ESI m/z calcd for C₈₂H₁₄₀N₁₀O₃ [M+H]⁺ 1841.94, found 1842.50.

### Example 229. Synthesis of (2S,4R)-tert-butyl 5-((8S,11S,12R,15S)-11,12-bis(4-(2,5-dioxo-2,5-dihydro-1H-pyrrol-1-yl)butanamido)-2,7,10,13,16,21-hexaoxo-8,15-bis(31-oxo-2,5,8,11,14,17,20,23,26,29-decaoxa-32-azahexatriacontan-36-yl)-3,4,5,6,7,8,9,10,11,12,13,14,15,16,17,18,19,20,21,22-icosahydro-2H-benzo[b][1,4,9,12,17,20]oxapentaazacyclotetracosin-24-yl)-4-((tert-butoxycarbonyl)amino)-2-methylpentanoate

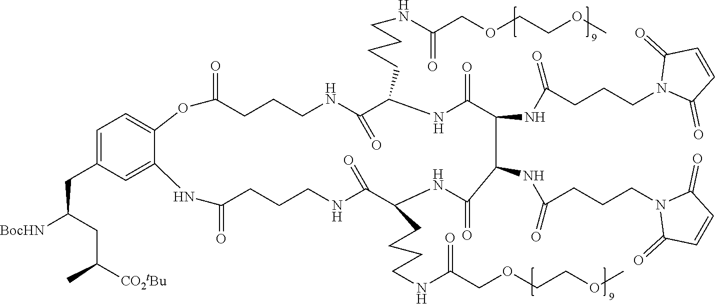

A mixture solution of (7S,10R,11S,14S)-10,11-bis(4-(2,5-dioxo-2,5-dihydro-1H-pyrrol-1-yl)butanamido)-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioic acid (154 mg, 0.0837 mmol) and (2S,4R)-tert-butyl 5-(3-amino-4-hydroxyphenyl)-4-((tert-butoxycarbonyl)amino)-2-methylpentanoate (33 mg, 0.0837 mmol) in DMF (6 mL) was cooled to 0° C. and HATU (64 mg, 0.167 mmol) and TEA (46 μL, 0.335 mmol) were added in sequence. The reaction was stirred for 1 h then diluted with water (100 mL), and extracted with EtOAc (3×100 mL). The EtOAc solution was washed with brine, dried over anhydrous Na₂SO₄, filtered, concentrated and purified by SiO₂ column chromatography (6:1 DCM/MeOH) to give the title compound (95 mg, 52% yield). MS ESI m/z calcd for C₁₀₃H₁₇₀N₁₂O₃₉ [M+H]⁺ 2200.17, found 2200.90.

### Example 230. Synthesis of (2S,4S)-5-((8S,11S,12R,15S)-11,12-bis(4-(2,5-dioxo-2,5-dihydro-1H-pyrrol-1-yl)butanamido)-2,7,10,13,16,21-hexaoxo-8,15-bis(31-oxo-2,5,8,11,14,17,20,23,26,29-decaoxa-32-azahexatriacontan-36-yl)-3,4,5,6,7,8,9,10,11,12,13,14,15,16,17,18,19,20,21,22-icosahydro-2H-benzo[b][1,4,9,12,17,20]oxapentaazacyclotetracosin-24-yl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic Acid (B-02)

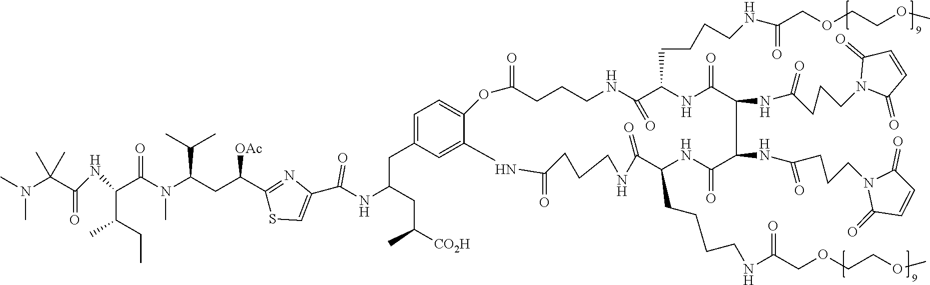

To a solution of (2S,4R)-tert-butyl 5-((8S,11S,12R,15S)-11,12-bis(4-(2,5-dioxo-2,5-dihydro-1H-pyrrol-1-yl)butanamido)-2,7,10,13,16,21-hexaoxo-8,15-bis(31-oxo-2,5,8,11,14,17,20,23,26,29-decaoxa-32-azahexatriacontan-36-yl)-3,4,5,6,7,8,9,10,11,12,13,14,15,16,17,18,19,20,21,22-icosahydro-2H-benzo[b][1,4,9,12,17,20]oxapentaazacyclotetracosin-24-yl)-4-((tert-butoxycarbonyl)amino)-2-methylpentanoate (98 mg, 0.045 mmol) in dichloromethane (3 mL) was added TFA (6 mL). The reaction mixture was stirred at r.t. for 1 h, and then concentrated and re-dissolved in DMA (1 mL), 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (31 mg, 0.045 mmol) and DIPEA (12 μL, 0.068 mmol) were added. The reaction mixture was stirred at r.t. for 90 min, then concentrated and purified by reverse phase HPLC (C₁₈ column, 10-100% acetonitrile/water) to afford the title compound (B-2) (36.2 mg, 62% yield). MS ESI m/z calcd for C₁₁₉H₁₉₄N₁₆O₄₂S [M+H]⁺ 1276.66, found 1276.65.

### Example 231. Synthesis of (S)-11-(5-(tert-butoxy)-2-((tert-butoxycarbonyl)amino)-5-oxopentanamido)undecanoic Acid

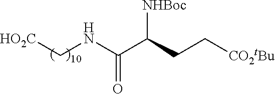

To a solution of Boc-Glu(OtBu)-OH (0.50 g, 1.65 mmol) in DMF (10 mL) were added HATU (0.69 g, 1.82 mmol) and TEA (0.26 mL, 1.82 mmol). After stirring for 30 min, a solution of 11-aminoundecanoic acid (0.33 g, 1.65 mmol) in DMF (10 mL) was added and the reaction was stirred at r.t. for 1h, then poured into a separatory funnel containing 200 mL of 1N HCl and extracted with DCM (3×50 mL). The organic phase was washed once with 100 mL of brine, then dried over anhydrous Na₂SO₄, filtered and concentrated. The residue was purified by column chromatography (MeOH/DCM) to afford the title compound (1.0 g, >100% yield). ESI: m/z: calcd for C₂₅H₄₇N₂O₇ [M+H]⁺: 487.33, found 487.34.

### Example 232. Synthesis of (S)-11-(2-amino-4-carboxybutanamido)undecanoic Acid

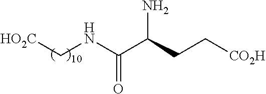

To a solution of (S)-11-(5-(tert-butoxy)-2-((tert-butoxycarbonyl)amino)-5-oxopentanamido)undecanoic acid (1.0 g, -2.05 mmol) in DCM (20 mL) was added TFA (5 mL). The reaction was stirred at room temperature for 30 min, then concentrated to dryness and dried twice with DCM. Finally, placed on a vacuum pump give the title compound (0.68 g, -2.06 mmol, ˜100% yield). ESI: m/z: calcd for C₁₆H₃₁N₂O₅ [M+H]⁺: 331.22, found 331.22.

### Example 233. Synthesis of (2S,4R)-tert-butyl 5-(3-(2-(((benzyloxy)carbonyl)amino)-3-methylbutanamido)-4-hydroxyphenyl)-4-((tert-butoxycarbonyl)amino)-2-methylpentanoate

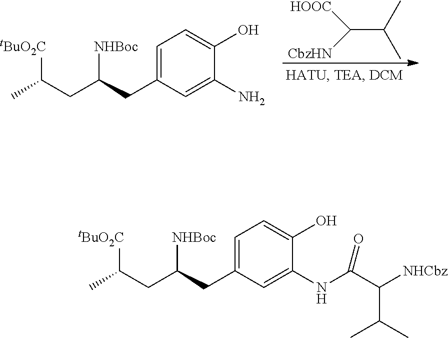 [SMILES: C[C@@H](C[C@H](Cc1ccc(O)c(N)c1)NC(=O)OC(C)(C)C)C(=O)OC(C)(C)C ; CC(C)C(NC(=O)OCc1ccccc1)C(=O)O ; CC(C)C(NC(=O)OCc1ccccc1)C(=O)Nc1cc(C[C@@H](C[C@H](C)C(=O)OC(C)(C)C)NC(=O)OC(C)(C)C)ccc1O]

(2S,4R)-tert-butyl 5-(3-amino-4-hydroxyphenyl)-4-((tert-butoxycarbonyl)amino)-2-methylpentanoate (0.2 g, 0.51 mmol), 2-(((benzyloxy)carbonyl)amino)-3-methylbutanoic acid (0.13 g, 0.51 mmol), HATU (0.20 g, 0.51 mmol) were dissolved in DCM (20 ml), followed by TEA (110 ul, 0.8 mmol) was added. The reaction mixture was stirred at RT overnight. Then the solvent was removed under reduced pressure and purified by SiO₂ column to give the title product (0.30 g, 91%). ESI: m/z: calcd for C₃₄H₅₀N₃O₈[M+H]⁺: 628.35, found 628.45.

### Example 234. Synthesis of (2S,4R)-tert-butyl 5-(3-(2-amino-3-methylbutanamido)-4-hydroxyphenyl)-4-((tert-butoxycarbonyl)amino)-2-methylpentanoate

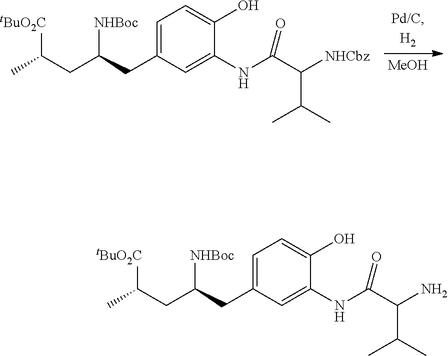 [SMILES: "MeOH" ; CC(C)C(NC(=O)OCc1ccccc1)C(=O)Nc1cc(C[C@@H](C[C@H](C)C(=O)OC(C)(C)C)NC(=O)OC(C)(C)C)ccc1O ; CC(C)C(N)C(=O)Nc1cc(C[C@@H](C[C@H](C)C(=O)OC(C)(C)C)NC(=O)OC(C)(C)C)ccc1O]

In a hydrogenation bottle, Pd/C (0.1 g, 33 wt %, 50% wet) was added to a solution (2S,4R)-tert-butyl 5-(3-(2-(((benzyloxy)carbonyl)amino)-3-methylbutanamido)-4-hydroxyphenyl)-4-((tert-butoxycarbonyl)amino)-2-methylpentanoate (0.29 g, 0.46 mmol) in MeOH (10 mL). The mixture was shaken overnight under 1 atm H₂, then filtered through Celite (filter aid). The filtrate was concentrated to afford the title compound (0.23 g, -100%) and used for next step without further purification. ESI: m/z: calcd for C₂₆H₄₄N₃O₆ [M+H]⁺:494.64, found 494.75.

### Example 235. Synthesis of (2S,4R)-tert-butyl 5-(3-(2-(2-(((benzyloxy)carbonyl)amino)propanamido)-3-methylbutanamido)-4-hydroxyphenyl)-4-((tert-butoxycarbonyl)amino)-2-methylpentanoate

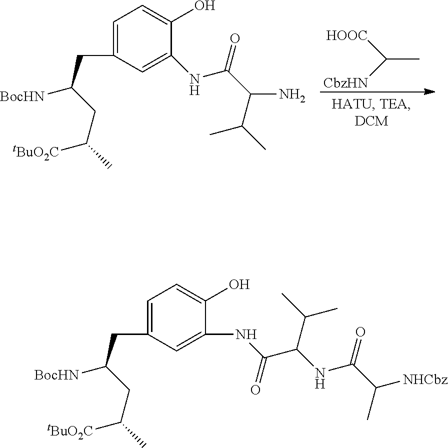 [SMILES: CC(C)C(N)C(=O)Nc1cc(C[C@@H](C[C@H](C)C(=O)OC(C)(C)C)NC(=O)OC(C)(C)C)ccc1O ; CC(NC(=O)OCc1ccccc1)C(=O)O ; CC(NC(=O)OCc1ccccc1)C(=O)NC(C(=O)Nc1cc(C[C@@H](C[C@H](C)C(=O)OC(C)(C)C)NC(=O)OC(C)(C)C)ccc1O)C(C)C]

(2S,4R)-tert-butyl 5-(3-(2-amino-3-methylbutanamido)-4-hydroxyphenyl)-4-((tert-butoxycarbonyl)amino)-2-methylpentanoate (0.23 g, 0.46 mmol), 2-(((benzyloxy)carbonyl)-aminopropanoic acid (0.10 g, 0.46 mmol) and HATU (0.18 g, 0.46 mmol) were dissolved in DCM (20 ml), followed by addition of TEA (110 ul, 0.8 mmol). The reaction mixture was stirred at RT overnight, concentrated under reduced pressure and purified on SiO₂ column to give the title product (0.3 g, 95%). ESI: m/z: calcd for C₃₇H₅₅N₄O₉ [M+H]⁺: 699.39, found 699.50.

### Example 236. Synthesis of (2S,4R)-tert-butyl 5-(3-(2-(2-aminopropanamido)-3-methylbutanamido)-4-hydroxyphenyl)-4-((tert-butoxycarbonyl)amino)-2-methylpentanoate

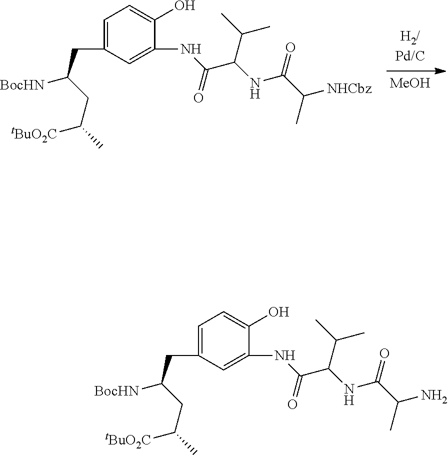 [SMILES: "MeOH" ; CC(NC(=O)OCc1ccccc1)C(=O)NC(C(=O)Nc1cc(C[C@@H](C[C@H](C)C(=O)OC(C)(C)C)NC(=O)OC(C)(C)C)ccc1O)C(C)C ; CC(N)C(=O)NC(C(=O)Nc1cc(C[C@@H](C[C@H](C)C(=O)OC(C)(C)C)NC(=O)OC(C)(C)C)ccc1O)C(C)C]

In a hydrogenation bottle, Pd/C (0.1 g, 33 wt %, 50% wet) was added to a solution of (2S,4R)-tert-butyl 5-(3-(2-(2-(((benzyloxy)carbonyl)amino)propanamido)-3-methylbutanamido)-4-hydroxyphenyl)-4-((tert-butoxycarbonyl)amino)-2-methylpentanoate (0.3 g, 0.43 mmol) in MeOH (10 mL). The mixture was shaken overnight under 1 atm H₂ then filtered through Celite (filter aid), the filtrate was concentrated to afford the title compound (0.22 g, 93%) which was used for the next step without further purification. ESI: m/z: calcd for C₂₉H₄₉N₄O₇ [M+H]⁺:565.35, found 565.60.

### Example 237. Synthesis of (2S,4R)-tert-butyl 5-((3S,6S,14R,15S)-14,15-bis(2-(2,5-dioxo-2,5-dihydro-1H-pyrrol-1-yl)acetamido)-3-isopropyl-6-methyl-2,5,8,13,16,21-hexaoxo-2,3,4,5,6,7,8,9,10,11,12,13,14,15,16,17,18,19,20,21-icosahydro-1H-benzo[b][1,4,7,10,15,20]oxapenta-azacyclotetracosin-25-yl)-4-((tert-butoxycarbonyl)amino)-2-methylpentanoate

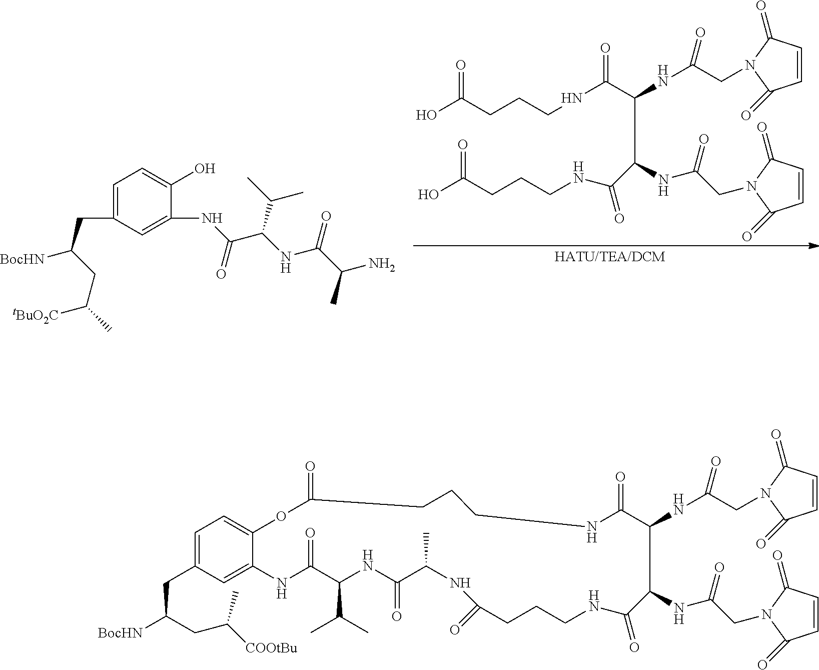

(2S,4R)-tert-butyl 5-(3-((S)-2-((S)-2-aminopropanamido)-3-methylbutanamido)-4-hydroxyphenyl)-4-((tert-butoxycarbonyl)amino)-2-methylpentanoate (0.150 g, 0.27 mmol), 4,4′-(((2R,3S)-2,3-bis(2-(2,5-dioxo-2,5-dihydro-1H-pyrrol-1-yl)acetamido)succinyl)bis(azanediyl))-dibutanoic acid (0.160 g, 0.270 mmol), HATU (0.402 g, 1.080 mmol) were dissolved in DCM (30 ml), followed by addition of TEA (55 ul, 0.4 mmol). The reaction mixture was stirred at RT overnight, concentrated under reduced pressure and purified on SiO₂ column (eluted with EtOAc/DCM, 1:10 to 1:5) to give the title product (0.187 g, 62%). ESI: m/z: calcd for C₅₃H₇₃N₁₀O₁₇ [M+H]⁺: 1121.51, found 1121.75.

### Example 238. Synthesis of (2S,4R)-4-amino-5-((3S,6S,14R,15S)-14,15-bis(2-(2,5-dioxo-2,5-dihydro-1H-pyrrol-1-yl)acetamido)-3-isopropyl-6-methyl-2,5,8,13,16,21-hexaoxo-2,3,4,5,6,7,8,9,10,11,12,13,14,15,16,17,18,19,20,21-icosahydro-1H-benzo[b][1,4,7,10,15,20]-oxapentaazacyclotetracosin-25-yl)-2-methylpentanoic Acid

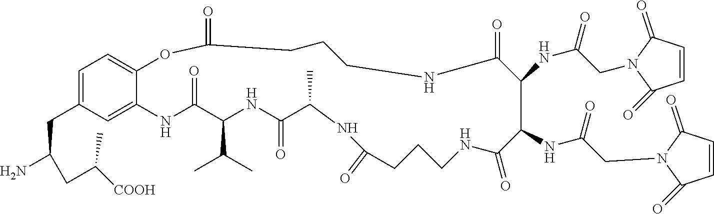 [SMILES: CC(C)[C@@H]1NC(=O)[C@H](C)NC(=O)CCCNC(=O)[C@H](NC(=O)CN2C(=O)C=CC2=O)[C@H](NC(=O)CN2C(=O)C=CC2=O)C(=O)NCCCC(=O)Oc2ccc(C[C@H](N)C[C@H](C)C(=O)O)cc2NC1=O]

(2S,4R)-tert-butyl 5-((3S,6S,14R,15S)-14,15-bis(2-(2,5-dioxo-2,5-dihydro-1H-pyrrol-1-yl)acetamido)-3-isopropyl-6-methyl-2,5,8,13,16,21-hexaoxo-2,3,4,5,6,7,8,9,10,11,12,13,14,15,16,17,18,19,20,21-icosahydro-1H-benzo[b][1,4,7,10,15,20]oxapenta-azacyclotetracosin-25-yl)-4-((tert-butoxycarbonyl)amino)-2-methylpentanoate (0.175 g, 0.156 mmol) was dissolved in DCM (6 ml), followed by addition of TFA (2 ml). The reaction mixture was stirred at RT for 2h, diluted with toluene (8 ml), concentrated to afford the title compound (150 mg, 100% yield) for the next step without further purification. ESI: m/z: calcd for C₄₄H₅₇N₁₀O₁₅ [M+H]⁺: 965.39, found 965.70.

### Example 239. Synthesis of 1-(((2S)-1-(((1R,3R)-1-acetoxy-1-(4-(((2R,4S)-1-((3S,6S,14R,15S)-14,15-bis(2-(2,5-dioxo-2,5-dihydro-1H-pyrrol-1-yl)acetamido)-3-isopropyl-6-methyl-2,5,8,13,16,21-hexaoxo-2,3,4,5,6,7,8,9,10,11,12,13,14,15,16,17,18,19,20,21-icosahydro-1H-benzo[b][1,4,7,10,15,20]oxapentaazacyclotetracosin-25-yl)-4-carboxypentan-2-yl)carbamoyl)thiazol-2-yl)-4-methylpentan-3-yl)(methyl)amino)-3-methyl-1-oxopentan-2-yl)amino)-N,N,N,2-tetramethyl-1-oxopropan-2-aminium (B-03)

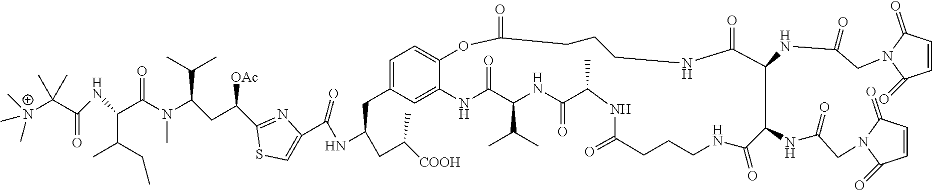 [SMILES: CCC(C)[C@H](NC(=O)C(C)(C)[N+](C)(C)C)C(=O)N(C)[C@H](C[C@@H](OC(C)=O)c1nc(C(=O)N[C@@H](Cc2ccc3c(c2)NC(=O)[C@H](C(C)C)NC(=O)[C@H](C)NC(=O)CCCNC(=O)[C@H](NC(=O)CN2C(=O)C=CC2=O)[C@H](NC(=O)CN2C(=O)C=CC2=O)C(=O)NCCCC(=O)O3)C[C@H](C)C(=O)O)cs1)C(C)C]

To the solution of (2S,4R)-4-amino-5-((3S,6S,14R,15S)-14,15-bis(2-(2,5-dioxo-2,5-dihydro-1H-pyrrol-1-yl)acetamido)-3-isopropyl-6-methyl-2,5,8,13,16,21-hexaoxo-2,3,4,5,6,7,8,9,10,11,12,13,14,15,16,17,18,19,20,21-icosahydro-1H-benzo[b][1,4,7,10,15,20]-oxapentaaza-cyclotetracosin-25-yl)-2-methylpentanoic acid (˜50 mg, 0.051 mmol) in DMA (4 ml) was added 1-(((2S)-1-(((1R,3R)-1-acetoxy-4-methyl-1-(4-((perfluorophenoxy)carbonyl)thiazol-2-yl)pentan-3-yl)(methyl)amino)-3-methyl-1-oxopentan-2-yl)amino)-N,N,N,2-tetramethyl-1-oxopropan-2-aminium (37 mg, 0.052 mmol) and DIPEA (3.4 ul, 0.02 mmol). The reaction mixture was stirred overnight, concentrated and purified on HPLC with a gradient of MeCN/H₂O (10% MeCN to 70% MeCN in 45 min, C-18 column, 10 mm (d)×250 mm (1), 9 ml/min) to give the title product (37.1 mg, 49% yield). ESI: m/z: calcd for C₇₀H₉₉N₁₄O₂₀S [M]⁺: 1487.69, found 1487.45.

### Example 240. Synthesis of (4R)-5-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaazacyclohexatetracontin-46-yl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic Acid (B-04)

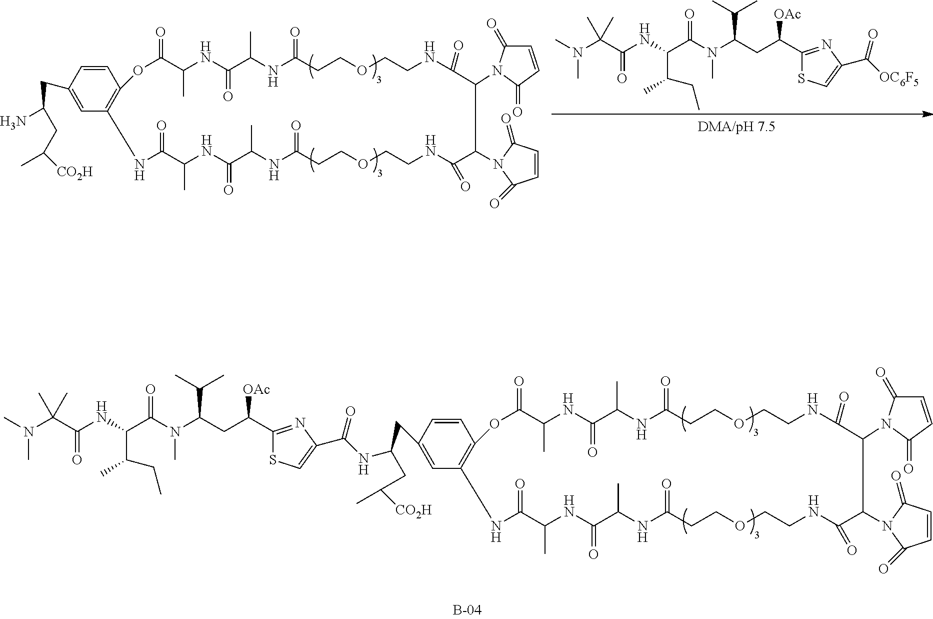

To the solution of (2R)-1-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaazacyclohexatetracontin-46-yl)-4-carboxypentan-2-aminium TFA salt (60 mg, 0.050 mmol) in DMA (15 ml) was added the pentafluo-actived acid compound (44 mg, 0.06 mmol) and 0.1 M NaH₂PO₄, pH 7.5, 8.0 m₁. The reaction mixture was stirred overnight, concentrated and purified on HPLC with a gradient of MeCN/H₂O (10% MeCN to 70% MeCN in 45 min, C-18 column, 10 mm (d)×250 mm (1), 8 ml/min) to give the title product B-4 (44 mg, 52% yield). ESI: m/z: calcd for C₇₉H₁₁₇N₁₄O₂₆S [M+H]⁺: 1709.79, found 1709.55.

### Example 241. Synthesis of (1R,3R)-1-(4-(((2R)-5-((2-aminoethyl)amino)-1-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaaza-cyclohexatetracontin-46-yl)-4-methyl-5-oxopentan-2-yl)carbamoyl)thiazol-2-yl)-3-((2S,3S)-2-(2-(dimethylamino)-2-methylpropanamido)-N,3-dimethylpentanamido)-4-methylpentyl acetate (B-5)

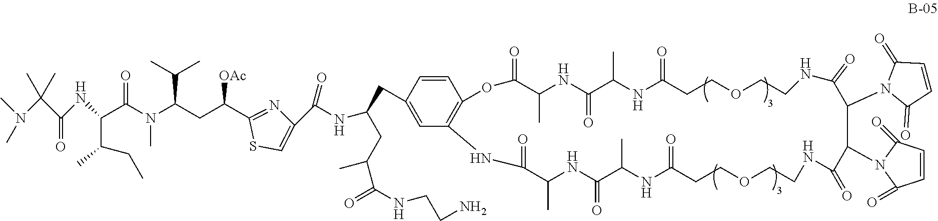

Compound B-4 (22.0 mg, 0.0129 mmol) in DMA (1 ml) was added EDC (15.0 mg, 0.078 mmol), ethane-1,2-diamine hydrochloride salt (8.0 mg, 0.060 mmol) and DIPEA (0.010 ml, 0.060 mmol). The mixture was stirred for overnight, concentrated, and purified by reverse phase HPLC (250 (L) mm x 10(d) mm, C₁₈ column, 10-100% acetonitrile/water in 40 min, v=8 ml/min) to afford the title compound (14.0 mg, 62% yield). ESI MS m/z: calcd for C₈₁H₁₂₃N₁₆O₂₅S [M+H]⁺ 1751.85, found 1751.20.

### Example 242. Synthesis of (1R,3R)-1-(4-(((28R)-1-amino-29-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaaza-cyclohexatetracontin-46-yl)-26-methyl-25-oxo-3,6,9,12,15,18,21-heptaoxa-24-azanonacosan-28-yl)carbamoyl)thiazol-2-yl)-3-((2S,3S)-2-(2-(dimethylamino)-2-methylpropanamido)-N,3-dimethylpentanamido)-4-methylpentyl acetate (B-06)

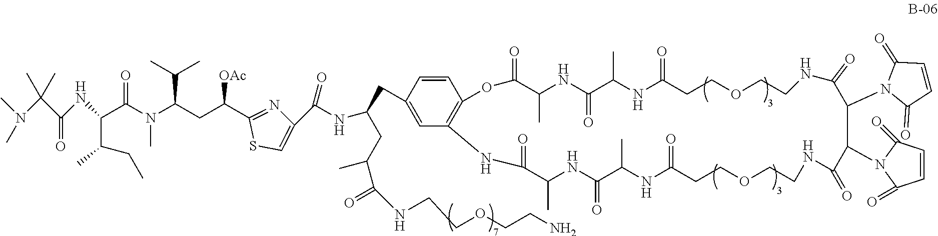

Compound B-4 (22.0 mg, 0.0129 mmol) in DMA (1 ml) was added EDC (15.0 mg, 0.078 mmol), 3,6,9,12,15,18,21-heptaoxatricosane-1,23-diamine hydrochloride salt (26.0 mg, 0.059 mmol) and DIPEA (0.010 ml, 0.060 mmol). The mixture was stirred for overnight, concentrated, and purified by reverse phase HPLC (250 (L) mm x 10(d) mm, C₁₈ column, 10-100% acetonitrile/water in 40 min, v=8 ml/min) to afford the title compound (14.5 mg, 55% yield). ESI MS m/z: calcd for C₉₅H₁₅₁N₁₆O₃₂S [M+H]⁺ 2060.03, found 2060.80.

### Example 243. Synthesis of (1R,3R)-1-(4-(((28R)-29-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaaza-cyclohexatetracontin-46-yl)-1-hydroxy-26-methyl-25-oxo-3,6,9,12,15,18,21-heptaoxa-24-azanonacosan-28-yl)carbamoyl)thiazol-2-yl)-3-((2S,3S)-2-(2-(dimethylamino)-2-methylpropanamido)-N,3-dimethylpentanamido)-4-methylpentyl acetate (B-07)

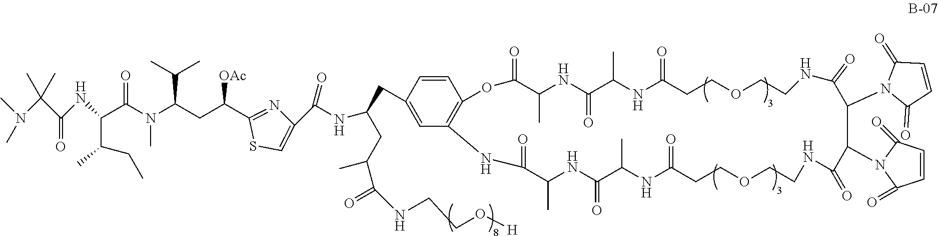

Compound B-4 (22.0 mg, 0.0129 mmol) in DMA (1 ml) was added EDC (15.0 mg, 0.078 mmol) and 23-amino-3,6,9,12,15,18,21-heptaoxatricosan-1-ol (22.0 mg, 0.059 mmol). The mixture was stirred for overnight, concentrated, and purified by reverse phase HPLC (250 (L) mm×10(d) mm, C₁₈ column, 10-100% acetonitrile/water in 40 min, v=8 ml/min) to afford the title compound (B-7) (14.1 mg, 53% yield). ESI MS m/z: calcd for C₉₅H₁₅₀N₁₅O₃₃S [M+H]⁺ 2061.02, found 2061.74.

### Example 244. Synthesis of (2S)-tert-butyl 2-((4R)-5-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxahepta-azacyclohexatetracontin-46-yl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanamido)-6-((tert-butoxycarbonyl)amino)hexanoate (B-08)

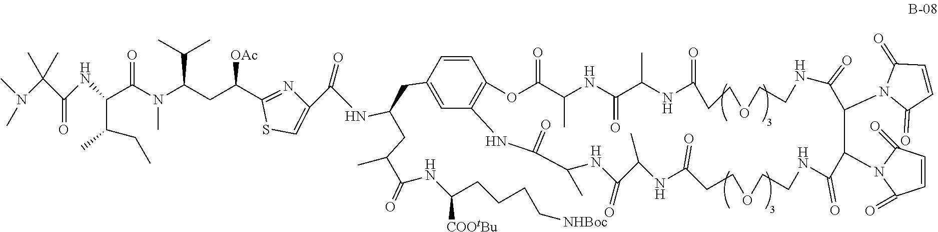

Compound B-4 (25.0 mg, 0.0146 mmol) in DMA (1 ml) was added EDC (15.0 mg, 0.078 mmol) and (S)-tert-butyl 2-amino-6-((tert-butoxycarbonyl)amino)hexanoate (9.0 mg, 0.030 mmol). The mixture was stirred for overnight, concentrated, and purified by reverse phase HPLC (250 (L) mm x 10(d) mm, C₁₈ column, 10-100% acetonitrile/water in 40 min, v=8 ml/min) to afford the title compound (20.5 mg, 71% yield). ESI MS m/z: calcd for C₉₄H₁₄₄N₁₆O₂₉S [M+H]⁺ 1994.00, found 1994.85.

### Example 245. Synthesis of (2S)-6-amino-2-((4R)-5-(22,23-bis(2,5-dioxo-2,5-dihydro-1H-pyrrol-1-yl)-3,6,39,42-tetramethyl-2,5,8,21,24,37,40,43-octaoxo-3,4,5,6,7,8,9,10,12,13,15,16,18,19,20,21,22,23,24,25,26,27,29,30,32,33,35,36,37,38,39,40,41,42,43,44-hexatriacontahydro-2H-benzo[b][1,14,17,20,31,34,37,4,7,10,23,28,41,44]heptaoxaheptaaza-cyclohexatetracontin-46-yl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanamido)hexanoic Acid (B-09)

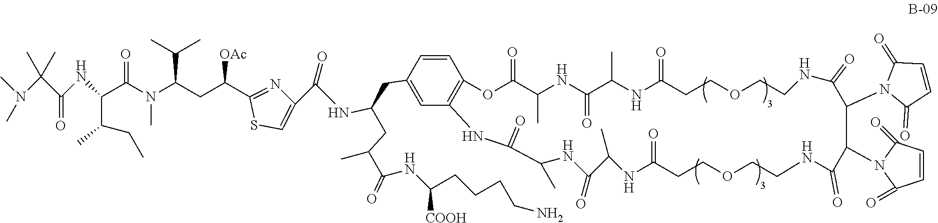

Compound B-8 (20.0 mg, 0.010 mmol) was dissolved in DCM (1 ml), followed by addition of TFA (1 ml). The reaction mixture was stirred at RT for 2h, then concentrated, and purified by reverse phase HPLC (250 (L) mm x 10(d) mm, C₁₈ column, 10-100% acetonitrile/water in 40 min, v=8 ml/min) to afford the title compound (13.5 mg, 73% yield). ESI: m/z: calcd for C₈₅H₁₂₉N₁₆O₂₇S [M+H]⁺: 1837.89, found 1838.20.

### Example 246. Synthesis of (S)-tert-butyl 39-amino-45-((5-((2R,4S)-5-(tert-butoxy)-2-((tert-butoxycarbonyl)amino)-4-methyl-5-oxopentyl)-2-hydroxyphenyl)amino)-11,21,33,40,45-pentaoxo-4,7,14,17,24,27,30-heptaoxa-10,20,34,41-tetraazapentatetracontan-1-oate

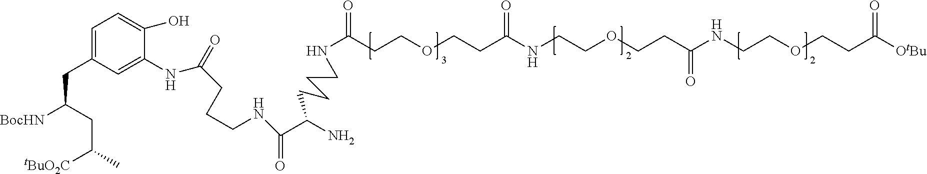

(S)-tert-butyl 5-((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-3,11,23,33-tetraoxo-1-phenyl-2,14,17,20,27,30,37,40-octaoxa-4,10,24,34-tetraazatritetracontan-43-oate (1.00 g, 0.742 mmol) in methanol (50 ml), was added Pd/C (10 wt %, 20 mg), then conducted with hydrogenated under 1 atm H₂ pressure with shanking overnight. The mixture was then filtered through Celite (filter aid), and the filtrate was concentrated to afford the title compound (900 mg, 100% yield). ESI m/z calcd for C₅₉H₁₀₄N₇O₁₉ [M+H]⁺: 1214.73, found 1214.90.

### Example 247. Synthesis of (42S,50S,51R)-42-((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-50,51-bis(3-(2,5-dioxo-2,5-dihydro-1H-pyrrol-1-yl)propanamido)-2,2-dimethyl-4,14,24,36,44,49,52-heptaoxo-3,7,10,17,20,27,30,33-octaoxa-13,23,37,43,48,53-hexaazaheptapentacontan-57-oic Acid and tert-butyl 38-((8S,16R,17S)-27-((2R,4S)-5-(tert-butoxy)-2-((tert-butoxycarbonyl)-amino)-4-methyl-5-oxopentyl)-16,17-bis(3-(2,5-dioxo-2,5-dihydro-1H-pyrrol-1-yl)propanamido)-2,7,10,15,18,23-hexaoxo-2,3,4,5,6,7,8,9,10,11,12,13,14,15,16,17,18,19,20,21,22,23-docosahydro-1H-benzo[b][1,4,9,12,17,22]oxapentaazacyclohexacosin-8-yl)-11,21,33-trioxo-4,7,14,17,24,27,30-heptaoxa-10,20,34-triazaoctatriacontan-1-oate

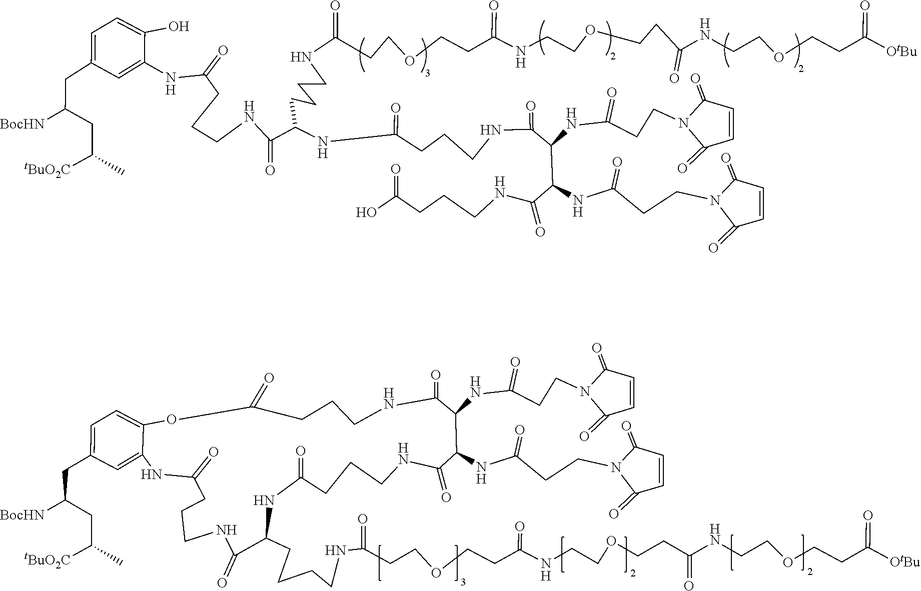

(S)-tert-butyl 39-amino-45-((5-((2R,4S)-5-(tert-butoxy)-2-((tert-butoxycarbonyl)amino)-4-methyl-5-oxopentyl)-2-hydroxyphenyl)amino)-11,21,33,40,45-pentaoxo-4,7,14,17,24,27,30-heptaoxa-10,20,34,41-tetraazapentatetracontan-1-oate (450 mg, 0.370 mmol) and 4,4′-(((2R,3S)-2,3-bis(3-(2,5-dioxo-2,5-dihydro-1H-pyrrol-1-yl)propanamido)succinyl)bis(azanediyl))dibutanoic acid (230 mg, 0.370 mmol) in DMA (40 ml) were added EDC (300 mg, 1.570 mmol) and DIPEA (100 mg, 0.775 mmol). The reaction mixture was stirred at RT overnight, concentrated under reduced pressure and purified on SiO₂ column (eluted with EtOAc/DCM, 1:10 to 1:5) to give (42S,50S,51R)-42-((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-50,51-bis(3-(2,5-dioxo-2,5-dihydro-1H-pyrrol-1-yl)propanamido)-2,2-dimethyl-4,14,24,36,44,49,52-heptaoxo-3,7,10,17,20,27,30,33-octaoxa-13,23,37,43,48,53-hexaazaheptapentacontan-57-oic acid (0.221 g, 33% yield). ESI: m/z: calcd for C₈₅H₁₃₄N₁₃O₃₀ [M+H]⁺: 1816.93, found 1817.25; and tert-butyl 38-((8S,16R,17S)-27-((2R,4S)-5-(tert-butoxy)-2-((tert-butoxycarbonyl)-amino)-4-methyl-5-oxopentyl)-16,17-bis(3-(2,5-dioxo-2,5-dihydro-1H-pyrrol-1-yl)propanamido)-2,7,10,15,18,23-hexaoxo-2,3,4,5,6,7,8,9,10,11,12,13,14,15,16,17,18,19,20,21,22,23-docosahydro-1H-benzo[b][1,4,9,12,17,22]oxapenta-azacyclohexacosin-8-yl)-11,21,33-trioxo-4,7,14,17,24,27,30-heptaoxa-10,20,34-triazaoctatriacontan-1-oate (0.260 g, 39% yield). ESI: m/z: calcd for C₈₅H₁₃₂N₁₃O₂₉ [M+H]⁺: 1797.92, found 1798.20.

### Example 248. Synthesis of (39S,47R,48S,56S)-di-tert-butyl 39,56-bis((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-47,48-bis(3-(2,5-dioxo-2,5-dihydro-1H-pyrrol-1-yl)propanamido)-11,21,33,41,46,49,54,62,74,84-decaoxo-4,7,14,17,24,27,30,65,68,71,78,81,88,91-tetradecaoxa-10,20,34,40,45,50,55,61,75,85-decaazatetranonacontane-1,94-dioate

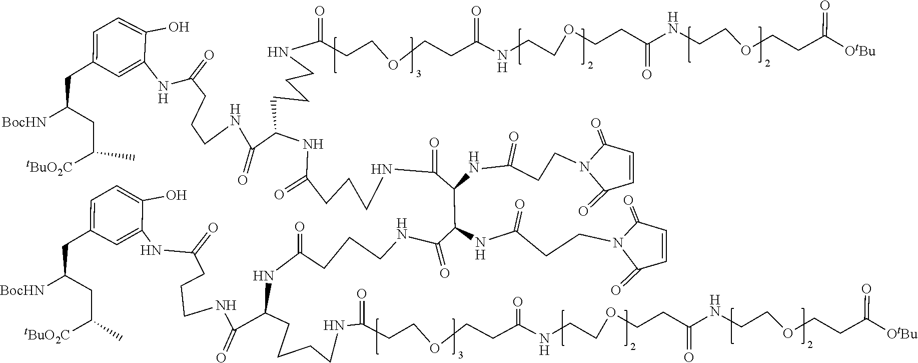

(S)-tert-butyl 39-amino-45-((5-((2R,4S)-5-(tert-butoxy)-2-((tert-butoxycarbonyl)amino)-4-methyl-5-oxopentyl)-2-hydroxyphenyl)amino)-11,21,33,40,45-pentaoxo-4,7,14,17,24,27,30-heptaoxa-10,20,34,41-tetraazapentatetracontan-1-oate (450 mg, 0.370 mmol) and 4,4′-(((2R,3S)-2,3-bis(3-(2,5-dioxo-2,5-dihydro-1H-pyrrol-1-yl)propanamido)succinyl)bis(azanediyl))dibutanoic acid (115 mg, 0.185 mmol) in DMA (40 ml) were added EDC (300 mg, 1.570 mmol). The reaction mixture was stirred at RT overnight, concentrated under reduced pressure and purified on SiO₂ column (eluted with EtOAc/DCM, 1:10 to 1:3) to give the title compound (0.378 g, 6800 yield). ESI: m/z: calcd for C₁₄₄H₂₃₅N₂₀O₄₈ [M+H]⁺: 3012.65, found 3012.95.

### Example 249. Synthesis of (33R,34S,42S)-tert-butyl 42-((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-33,34-bis(3-(2,5-dioxo-2,5-dihydro-1H-pyrrol-1-yl)propanamido)-27,32,35,40,48,60,70-heptaoxo-2,5,8,11,14,17,20,23,51,54,57,64,67,74,77-pentadecaoxa-26,31,36,41,47,61,71-heptaazaoctacontan-80-oate

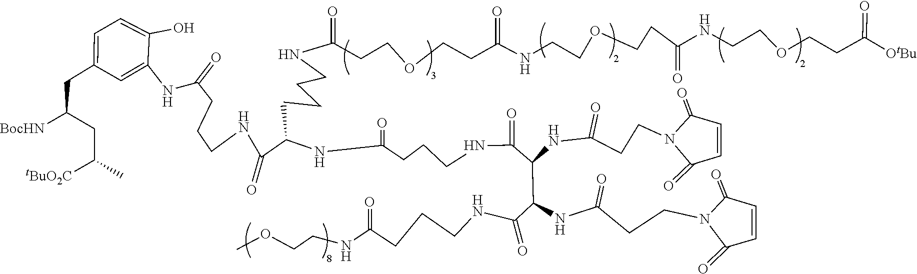

(42S,50S,51R)-42-((4-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-50,51-bis(3-(2,5-dioxo-2,5-dihydro-1H-pyrrol-1-yl)propanamido)-2,2-dimethyl-4,14,24,36,44,49,52-heptaoxo-3,7,10,17,20,27,30,33-octaoxa-13,23,37,43,48,53-hexaazaheptapentacontan-57-oic acid (100 mg, 0.055 mmol) in DMA (30 ml) were added 2,5,8,11,14,17,20,23-octaoxapentacosan-25-amine, HCl salt (30 mg, 0.071 mmol) and EDC (25 mg, 0.130 mmol). The reaction mixture was stirred at RT overnight, concentrated under reduced pressure and purified on SiO₂ column (eluted with EtOAc/DCM, 1:8 to 1:3) to give the title compound (92.2 mg, 76% yield). ESI: m/z: calcd for C₁₀₂H₁₆₉N₁₄O₃₇ [M+H]⁺: 2182.17, found 2182.95.

### Example 250. Synthesis of (38S,46S,47R)-38-((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-46,47-bis(3-(2,5-dioxo-2,5-dihydro-1H-pyrrol-1-yl)propanamido)-32,40,45,48-tetraoxo-2,5,8,11,14,17,20,23,26,29-decaoxa-33,39,44,49-tetraazatripentacontan-53-oic acid and (2S,4R)-tert-butyl 5-((8S,16R,17S)-16,17-bis(3-(2,5-dioxo-2,5-dihydro-1H-pyrrol-1-yl)propanamido)-2,7,10,15,18,23-hexaoxo-8-(30-oxo-2,5,9,12,15,18,21,24,27-nonaoxa-31-azapentatriacontan-35-yl)-2,3,4,5,6,7,8,9,10,11,12,13,14,15,16,17,18,19,20,21,22,23-docosahydro-1H-benzo[b][1,4,9,12,17,22]-oxapentaazacyclohexacosin-27-yl)-4-((tert-butoxycarbonyl)amino)-2-methylpentanoate

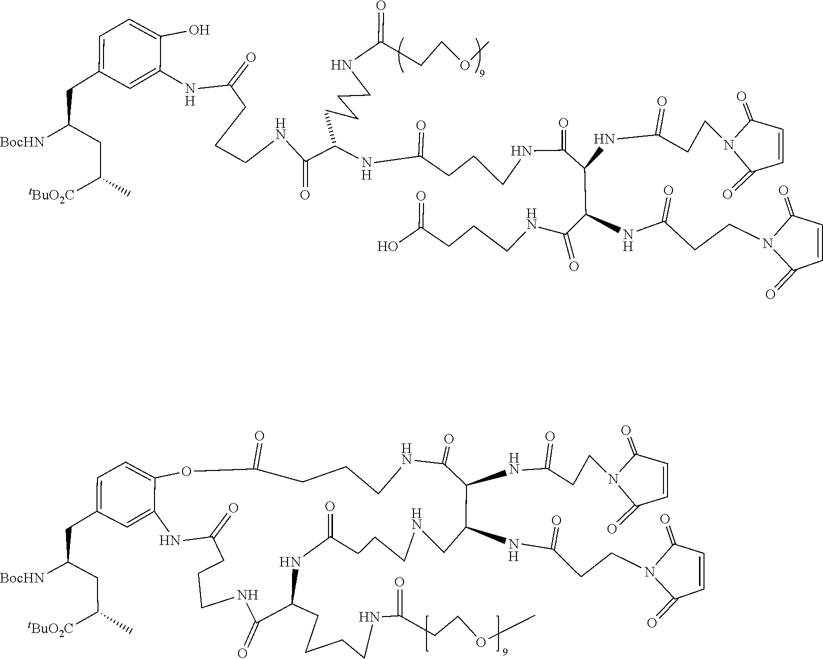

(2S,4R)-tert-butyl 5-(3-((S)-36-amino-30,37-dioxo-2,5,9,12,15,18,21,24,27-nonaoxa-31,38-diazadotetracontanamido)-4-hydroxyphenyl)-4-((tert-butoxycarbonyl)amino)-2-methylpentanoate (400 mg, 0.377 mmol) and 4,4′-(((2R,3S)-2,3-bis(3-(2,5-dioxo-2,5-dihydro-1H-pyrrol-1-yl)propanamido)succinyl)bis(azanediyl))dibutanoic acid (234 mg, 0.377 mmol) in DMA (50 ml) were added EDC (300 mg, 1.570 mmol) and DIPEA (100 mg, 0.775 mmol). The reaction mixture was stirred at RT overnight, concentrated under reduced pressure and purified on SiO₂ column (eluted with EtOAc/DCM, 1:10 to 1:5) to afford (38S,46S,47R)-38-((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-46,47-bis(3-(2,5-dioxo-2,5-dihydro-1H-pyrrol-1-yl)propanamido)-32,40,45,48-tetraoxo-2,5,8,11,14,17,20,23,26,29-decaoxa-33,39,44,49-tetraazatripentacontan-53-oic acid (0.192 g, 31% yield). ESI: m/z: calcd for C₇₈H₁₂₄N₁₁O₂₈ [M+H]⁺: 1662.85, found 1662.60; and (2S,4R)-tert-butyl 5-((8S,16R,17S)-16,17-bis(3-(2,5-dioxo-2,5-dihydro-1H-pyrrol-1-yl)propanamido)-2,7,10,15,18,23-hexaoxo-8-(30-oxo-2,5,9,12,15,18,21,24,27-nonaoxa-31-azapentatriacontan-35-yl)-2,3,4,5,6,7,8,9,10,11,12,13,14,15,16,17,18,19,20,21,22,23-docosahydro-1H-benzo[b][1,4,9,12,17,22]-oxapentaazacyclohexacosin-27-yl)-4-((tert-butoxycarbonyl)amino)-2-methylpentanoate (0.260 g, 39% yield). ESI: m/z: calcd for C₇₈H₁₂₂N₁₁O₂₇ [M+H]⁺: 1644.84, found 1645.25.

### Example 251. Synthesis of (2S,2'S,4R,4′R)-di-tert-butyl 5,5′-((((7S,15R,16S,24S)-15,16-bis(3-(2,5-dioxo-2,5-dihydro-1H-pyrrol-1-yl)propanamido)-6,9,14,17,22,25-hexaoxo-7,24-bis(30-oxo-2,5,9,12,15,18,21,24,27-nonaoxa-31-azapentatriacontan-35-yl)-5,8,13,18,23,26-hexaazatriacontane-1,30-dioyl)bis(azanediyl))bis(4-hydroxy-3,1-phenylene))bis(4-((tert-butoxycarbonyl)amino)-2-methylpentanoate)

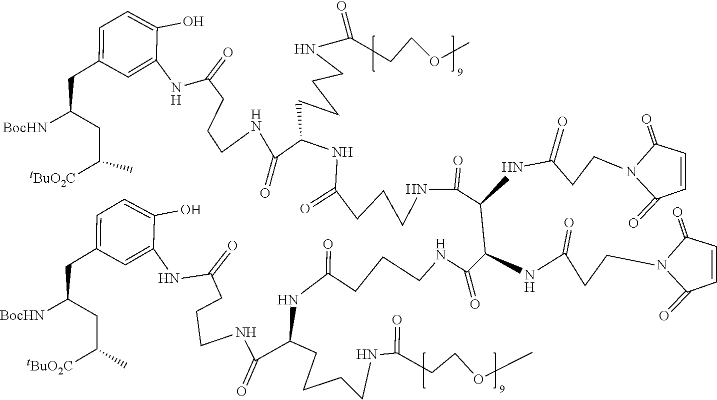

(2S,4R)-tert-butyl 5-(3-((S)-36-amino-30,37-dioxo-2,5,9,12,15,18,21,24,27-nonaoxa-31,38-diazadotetracontanamido)-4-hydroxyphenyl)-4-((tert-butoxycarbonyl)amino)-2-methylpentanoate (400 mg, 0.377 mmol) and 4,4′-(((2R,3S)-2,3-bis(3-(2,5-dioxo-2,5-dihydro-1H-pyrrol-1-yl)propanamido)succinyl)bis(azanediyl))dibutanoic acid (115 mg, 0.185 mmol) in DMA (50 ml) were added EDC (300 mg, 1.570 mmol). The reaction mixture was stirred at RT overnight, concentrated under reduced pressure and purified on SiO₂ column (eluted with EtOAc/DCM, 1:10 to 1:3) to give the title compound (0.325 g, 65% yield). ESI: m/z: calcd for C₁₃₀H₂₁₅N₁₆O₄₄ [M+H]⁺: 2704.50, found 2704.90.

### Example 252. Synthesis of (2S,4R)-tert-butyl 5-(3-((34R,35S,43S)-34,35-bis(3-(2,5-dioxo-2,5-dihydro-1H-pyrrol-1-yl)propanamido)-1-hydroxy-28,33,36,41,44-pentaoxo-43-(32-oxo-2,5,8,11,14,17,20,23,26,29-decaoxa-33-azaheptatriacontan-37-yl)-3,6,9,12,15,18,21,24-octaoxa-27,32,37,42,45-pentaazanonatetracontanamido)-4-hydroxyphenyl)-4-((tert-butoxycarbonyl)amino)-2-methylpentanoate

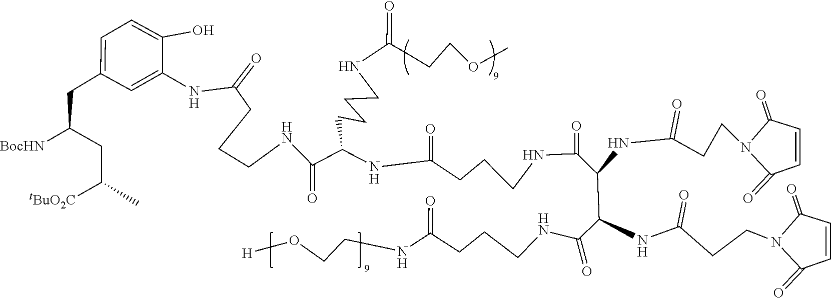

(38S,46S,47R)-38-((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-46,47-bis(3-(2,5-dioxo-2,5-dihydro-1H-pyrrol-1-yl)propanamido)-32,40,45,48-tetraoxo-2,5,8,11,14,17,20,23,26,29-decaoxa-33,39,44,49-tetraazatripentacontan-53-oic acid (100 mg, 0.060 mmol) in DMA (30 ml) were added 26-amino-3,6,9,12,15,18,21,24-octaoxahexacosan-1-ol, HCl salt (31 mg, 0.069 mmol) and EDC (35 mg, 0.183 mmol). The reaction mixture was stirred at RT overnight, concentrated under reduced pressure and purified on SiO₂ column (eluted with EtOAc/DCM, 1:8 to 1:3) to give the title compound (86.5 mg, 69% yield). ESI: m/z: calcd for C₉₇H₁₆₃N₁₂O₃₇ [M+H]⁺: 2088.12, found 2088.85.

### Example 253. Synthesis of (39S,47R,48S)-39-((4-((5-((2R,4S)-2-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-4-carboxypentyl)-2-hydroxyphenyl)amino)-4-oxobutyl)carbamoyl)-47,48-bis(3-(2,5-dioxo-2,5-dihydro-1H-pyrrol-1-yl)propanamido)-11,21,33,41,46,49-hexaoxo-4,7,14,17,24,27,30-heptaoxa-10,20,34,40,45,50-hexaazatetrapentacontane-1,54-dioic acid (B-10)

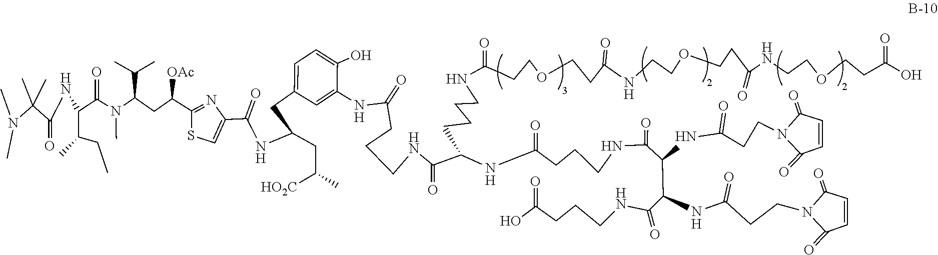

(42S,50S,51R)-42-((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-50,51-bis(3-(2,5-dioxo-2,5-dihydro-1H-pyrrol-1-yl)propanamido)-2,2-dimethyl-4,14,24,36,44,49,52-heptaoxo-3,7,10,17,20,27,30,33-octaoxa-13,23,37,43,48,53-hexaazaheptapentacontan-57-oic acid (0.120 g, 0.066 mmol) was dissolved in DCM (6 ml), followed by addition of TFA (2 ml). The reaction mixture was stirred at RT for 45 min, diluted with toluene (8 ml), concentrated to afford (39S,47R,48S)-39-((4-((5-((2R,4S)-2-amino-4-carboxypentyl)-2-hydroxyphenyl)amino)-4-oxobutyl)carbamoyl)-47,48-bis(3-(2,5-dioxo-2,5-dihydro-1H-pyrrol-1-yl)propanamido)-11,21,33,41,46,49-hexaoxo-4,7,14,17,24,27,30-heptaoxa-10,20,34,40,45,50-hexaazatetrapenta-contane-1,54-dioic acid, TFA salt (106 mg, ˜100% yield) for the next step without further purification. Then the compound in DMA (15 ml) was added perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (46 mg, 0.066 mmol) and DIPEA (10 ul, 0.055 mmol). The reaction mixture was stirred overnight, concentrated and purified on HPLC with a gradient of MeCN/H₂O (10% MeCN to 70% MeCN in 45 min, C-18 column, 20 mm (d)×250 mm (1), 9 ml/min) to give the title product (64.1 mg, 46% yield). ESI: m/z: calcd for C₉₇H₁₅₀N₁₇O₃₃S [M+H]⁺: 2113.02, found 2113.80.

### Example 254. Synthesis of 38-((8S,16R,17S)-27-((2R,4S)-2-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-4-carboxypentyl)-16,17-bis(3-(2,5-dioxo-2,5-dihydro-1H-pyrrol-1-yl)propan-amido)-2,7,10,15,18,23-hexaoxo-2,3,4,5,6,7,8,9,10,11,12,13,14,15,16,17,18,19,20,21,22,23-docosahydro-1H-benzo[b][1,4,9,12,17,22]oxapentaazacyclohexacosin-8-yl)-11,21,33-trioxo-4,7,14,17,24,27,30-heptaoxa-10,20,34-triazaoctatriacontan-1-oic Acid (B-11)

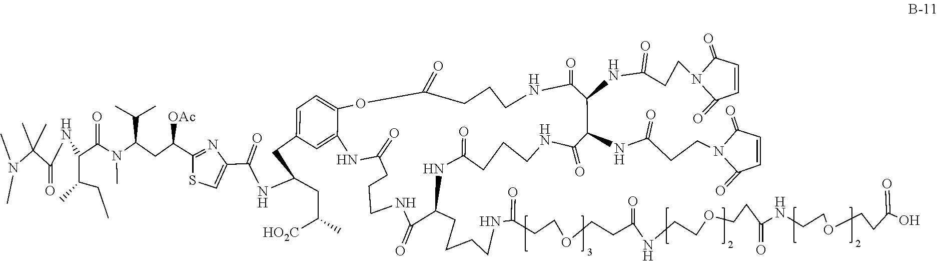

Tert-butyl 38-((8S,16R,17S)-27-((2R,4S)-5-(tert-butoxy)-2-((tert-butoxycarbonyl)-amino)-4-methyl-5-oxopentyl)-16,17-bis(3-(2,5-dioxo-2,5-dihydro-1H-pyrrol-1-yl)propanamido)-2,7,10,15,18,23-hexaoxo-2,3,4,5,6,7,8,9,10,11,12,13,14,15,16,17,18,19,20,21,22,23-docosahydro-1H-benzo[b][1,4,9,12,17,22]oxapenta-azacyclohexacosin-8-yl)-11,21,33-trioxo-4,7,14,17,24,27,30-heptaoxa-10,20,34-triazaoctatriacontan-1-oate (0.150 g, 0.083 mmol) was dissolved in DCM (6 ml), followed by addition of TFA (2 ml). The reaction mixture was stirred at RT for 45 min, diluted with toluene (8 ml), concentrated to afford 38-((8S,16R,17S)-27-((2R,4S)-2-amino-4-carboxypentyl)-16,17-bis(3-(2,5-dioxo-2,5-dihydro-1H-pyrrol-1-yl)propanamido)-2,7,10,15,18,23-hexaoxo-2,3,4,5,6,7,8,9,10,11,12,13,14,15,16,17,18,19,20,21,22,23-docosahydro-1H-benzo[b][1,4,9,12,17,22]oxapentaazacyclohexacosin-8-yl)-11,21,33-trioxo-4,7,14,17,24,27,30-heptaoxa-10,20,34-triazaoctatriacontan-1-oic acid, TFA salt (135 mg, ˜101% yield) for the next step without further purification. Then the compound in DMA (15 ml) was added perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (60 mg, 0.084 mmol) and DIPEA (10 ul, 0.055 mmol). The reaction mixture was stirred overnight, concentrated and purified on HPLC with a gradient of MeCN/H₂O (10% MeCN to 70% MeCN in 45 min, C-18 column, 20 mm (d)×250 mm (1), 9 ml/min) to give the title product (81.6 mg, 47% yield). ESI: m/z: calcd for C₉₇H₁₄₉N₁₇O₃₂S [M+H]⁺: 2095.01, found 2095.65.

### Example 255. Synthesis of Compound B-12 Structure Shown Below

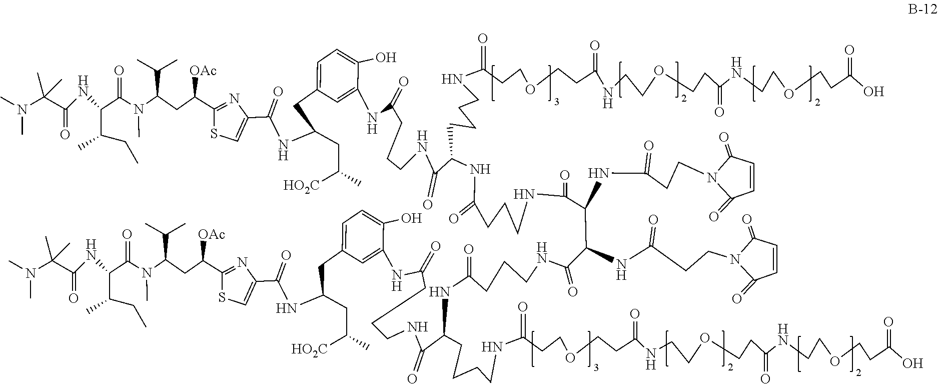

(39S,47R,48S,56S)-di-tert-butyl 39,56-bis((4-((5-((2R,4S)-5-(tert-butoxy)-2-((tert-butoxycarbonyl)amino)-4-methyl-5-oxopentyl)-2-hydroxyphenyl)amino)-4-oxobutyl)carbamoyl)-47,48-bis(3-(2,5-dioxo-2,5-dihydro-1H-pyrrol-1-yl)propanamido)-11,21,33,41,46,49,54,62,74,84-decaoxo-4,7,14,17,24,27,30,65,68,71,78,81,88,91-tetradecaoxa-10,20,34,40,45,50,55,61,75,85-decaazatetranonacontane-1,94-dioate (175 mg, 0.058 mmol) was dissolved in DCM (6 ml), followed by addition of TFA (2 ml). The reaction mixture was stirred at RT for 45 min, diluted with toluene (8 ml), concentrated to afford (39S,47R,48S,56S)-39,56-bis((4-((5-((2R,4S)-2-amino-4-carboxypentyl)-2-hydroxyphenyl)amino)-4-oxobutyl)carbamoyl)-47,48-bis(3-(2,5-dioxo-2,5-dihydro-1H-pyrrol-1-yl)propanamido)-11,21,33,41,46,49,54,62,74,84-decaoxo-4,7,14,17,24,27,30,65,68,71,78,81,88,91-tetradecaoxa-10,20,34,40,45,50,55,61,75,85-decaazatetranonacontane-1,94-dioic acid, TFA salt (151 mg, 99% yield). Then the compound in DMA (15 ml) was added perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (85 mg, 0.123 mmol) and DIPEA (18 ul, 0.103 mmol). The reaction mixture was stirred overnight, concentrated and purified on HPLC with a gradient of MeCN/H₂O (10% MeCN to 70% MeCN in 45 min, C-18 column, 20 mm (d)×250 mm (1), 9 ml/min) to give the title product (81.6 mg, 47% yield). ESI: m/z: calcd for C₁₆₈H₂₆₇N₂₈O₅₄S₂[M+H]⁺: 3604.84, found 3604.80.

### Example 256. Synthesis of (36S,44S,45R)-36-((4-((5-((2R,4S)-2-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-4-carboxypentyl)-2-hydroxyphenyl)amino)-4-oxobutyl)carbamoyl)-44,45-bis(3-(2,5-dioxo-2,5-dihydro-1H-pyrrol-1-yl)propanamido)-30,38,43,46-tetraoxo-2,5,9,12,15,18,21,24,27-nonaoxa-31,37,42,47-tetraazahenpentacontan-51-oic Acid (B-13)

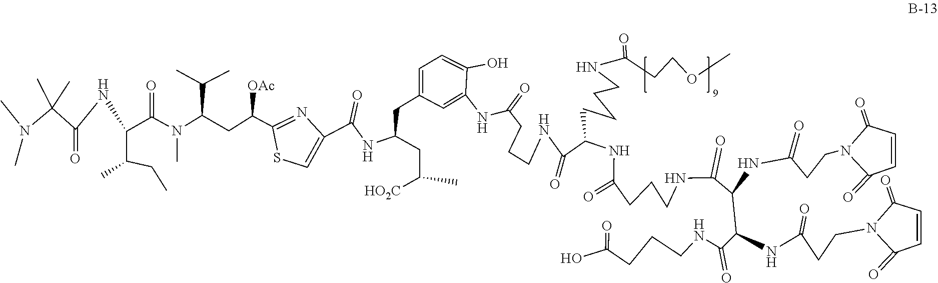

(2S,4R)-tert-butyl 5-((8S,16R,17S)-16,17-bis(3-(2,5-dioxo-2,5-dihydro-1H-pyrrol-1-yl)propanamido)-2,7,10,15,18,23-hexaoxo-8-(30-oxo-2,5,9,12,15,18,21,24,27-nonaoxa-31-azapentatriacontan-35-yl)-2,3,4,5,6,7,8,9,10,11,12,13,14,15,16,17,18,19,20,21,22,23-docosahydro-1H-benzo[b][1,4,9,12,17,22]-oxapentaazacyclohexacosin-27-yl)-4-((tert-butoxycarbonyl)amino)-2-methylpentanoate (0.175 g, 0.152 mmol) was dissolved in DCM (6 ml), followed by addition of TFA (2 ml). The reaction mixture was stirred at RT for 1h, diluted with toluene (8 ml), concentrated to afford (36S,44R,45S)-36-((4-((5-((2R,4S)-2-amino-4-carboxypentyl)-2-hydroxyphenyl)amino)-4-oxobutyl)carbamoyl)-44,45-bis(3-(2,5-dioxo-2,5-dihydro-1H-pyrrol-1-yl)propanamido)-30,38,43,46-tetraoxo-2,5,9,12,15,18,21,24,27-nonaoxa-31,37,42,47-tetraazahenpentacontan-51-oic acid (230 mg, 101% yield) for the next step without further purification. Then the compound in DMA (15 ml) was added perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (106 mg, 0.152 mmol) and DIPEA (20 ul, 0.115 mmol). The reaction mixture was stirred overnight, concentrated and purified on HPLC with a gradient of MeCN/H₂O (10% MeCN to 70% MeCN in 45 min, C-18 column, 20 mm (d)×250 mm (1), 9 ml/min) to give the title product (149.1 mg, 49% yield). ESI: m/z: calcd for C₉₄H₁₄₈N₁₅O₃₁S [M+H]⁺: 2015.01, found 2015.65.

### Example 257. Synthesis of (2S,4R)-5-(3-((34R,35S,43S)-34,35-bis(3-(2,5-dioxo-2,5-dihydro-1H-pyrrol-1-yl)propanamido)-1-hydroxy-28,33,36,41,44-pentaoxo-43-(32-oxo-2,5,8,11,14,17,20,23,26,29-decaoxa-33-azaheptatriacontan-37-yl)-3,6,9,12,15,18,21,24-octaoxa-27,32,37,42,45-pentaazanonatetracontanamido)-4-hydroxyphenyl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic Acid (B-14)

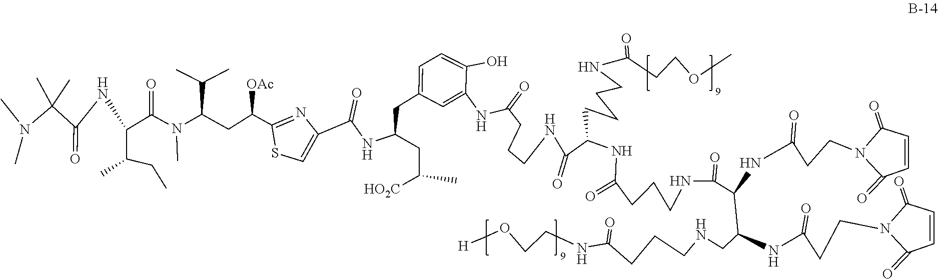

(2S,4R)-tert-butyl 5-(3-((34R,35S,43S)-34,35-bis(3-(2,5-dioxo-2,5-dihydro-1H-pyrrol-1-yl)propanamido)-1-hydroxy-28,33,36,41,44-pentaoxo-43-(32-oxo-2,5,8,11,14,17,20,23,26,29-decaoxa-33-azaheptatriacontan-37-yl)-3,6,9,12,15,18,21,24-octaoxa-27,32,37,42,45-pentaazanonatetracontanamido)-4-hydroxyphenyl)-4-((tert-butoxycarbonyl)amino)-2-methylpentanoate (0.085 g, 0.040 mmol) was dissolved in DCM (6 ml), followed by addition of TFA (2 ml). The reaction mixture was stirred at RT for 1h, diluted with toluene (8 ml), concentrated to afford 2,5,8,11,14,17,20,23,26,29-decaoxa-33-azaheptatriacontan-37-yl)-3,6,9,12,15,18,21,24-octaoxa-27,32,37,42,45-pentaazanonatetracontanamido)-4-hydroxyphenyl)-2-methylpentanoic acid, TFA salt (78 mg, 100% yield) for the next step without further purification. Then the compound in DMA (15 ml) was added perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (40 mg, 0.056 mmol) and DIPEA (7 ul, 0.040 mmol). The reaction mixture was stirred overnight, concentrated and purified on HPLC with a gradient of MeCN/H₂O (10% MeCN to 70% MeCN in 45 min, C-18 column, 20 mm (d)×250 mm (1), 9 ml/min) to give the title product (51.3 mg, 52% yield). ESI: m/z: calcd for C₁₁₃H₁₈₇N₁₆O₄₀S [M+H]⁺: 2440.27, found 2440.90.

### Example 258. Synthesis of (2S,4R)-5-((8S,16R,17S)-16,17-bis(3-(2,5-dioxo-2,5-dihydro-1H-pyrrol-1-yl)propanamido)-2,7,10,15,18,23-hexaoxo-8-(30-oxo-2,5,9,12,15,18,21,24,27-nonaoxa-31-azapentatriacontan-35-yl)-2,3,4,5,6,7,8,9,10,11,12,13,14,15,16,17,18,19,20,21,22,23-docosahydro-1H-benzo[b][1,4,9,12,17,22]oxapentaazacyclohexacosin-27-yl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic Acid (B-15)

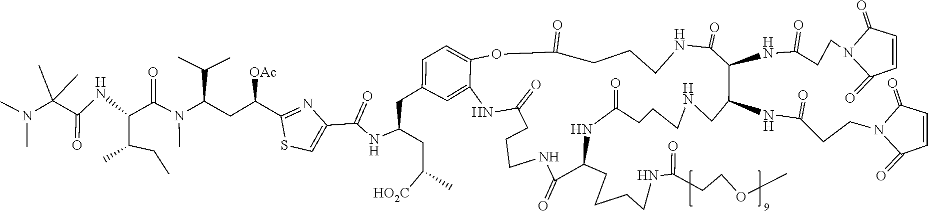

(2S,4R)-tert-butyl 5-((8S,16R,17S)-16,17-bis(3-(2,5-dioxo-2,5-dihydro-1H-pyrrol-1-yl)propanamido)-2,7,10,15,18,23-hexaoxo-8-(30-oxo-2,5,9,12,15,18,21,24,27-nonaoxa-31-azapentatriacontan-35-yl)-2,3,4,5,6,7,8,9,10,11,12,13,14,15,16,17,18,19,20,21,22,23-docosahydro-1H-benzo[b][1,4,9,12,17,22]-oxapentaazacyclohexacosin-27-yl)-4-((tert-butoxycarbonyl)amino)-2-methylpentanoate (0.145 g, 0.0882 mmol) was dissolved in DCM (6 ml), followed by addition of TFA (2 ml). The reaction mixture was stirred at RT for 1h, diluted with toluene (8 ml), concentrated to afford (2S,4R)-4-amino-5-((8S,16R,17S)-16,17-bis(3-(2,5-dioxo-2,5-dihydro-1H-pyrrol-1-yl)propanamido)-2,7,10,15,18,23-hexaoxo-8-(30-oxo-2,5,9,12,15,18,21,24,27-nonaoxa-31-azapentatriacontan-35-yl)-2,3,4,5,6,7,8,9,10,11,12,13,14,15,16,17,18,19,20,21,22,23-docosahydro-1H-benzo[b][1,4,9,12,17,22]oxapentaaza-cyclohexacosin-27-yl)-2-methylpentanoic acid, TFA salt (133 mg, 101% yield) for the next step without further purification. Then the compound in DMA (15 ml) was added perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (62 mg, 0.0885 mmol) and DIPEA (15 ul, 0.086 mmol). The reaction mixture was stirred overnight, concentrated and purified on HPLC with a gradient of MeCN/H₂O (10% MeCN to 70% MeCN in 45 min, C-18 column, 20 mm (d)×250 mm (1), 9 ml/min) to give the title product (83.1 mg, 47% yield). ESI: m/z: calcd for C₉₄H₁₄₆N₁₅O₃₀S [M+H]⁺: 1997.00, found 1997.60.

### Example 259. Synthesis of (S,S,R,R,2S,2′S,4R,4′R)-5,5′-((((7S,15R,16S,24S)-15,16-bis(3-(2,5-dioxo-2,5-dihydro-1H-pyrrol-1-yl)propanamido)-6,9,14,17,22,25-hexaoxo-7,24-bis(30-oxo-2,5,9,12,15,18,21,24,27-nonaoxa-31-azapentatriacontan-35-yl)-5,8,13,18,23,26-hexaazatriacontane-1,30-dioyl)bis(azanediyl))bis(4-hydroxy-3,1-phenylene))bis(4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid) (B-16)

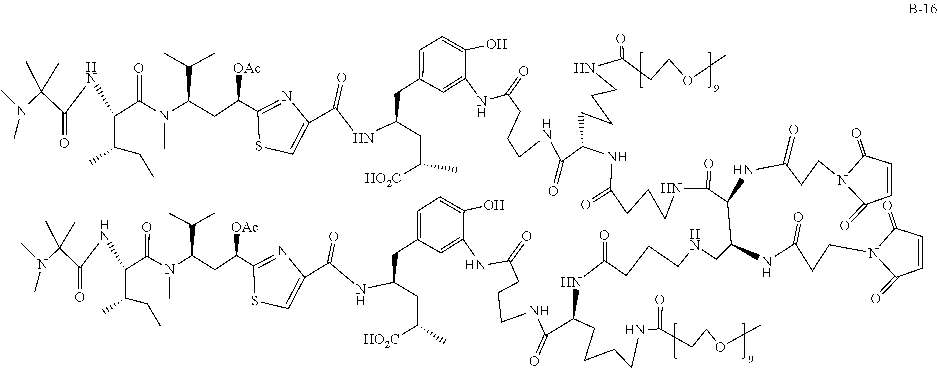

(2S,2'S,4R,4′R)-di-tert-butyl 5,5′-((((7S,15R,16S,24S)-15,16-bis(3-(2,5-dioxo-2,5-dihydro-1H-pyrrol-1-yl)propanamido)-6,9,14,17,22,25-hexaoxo-7,24-bis(30-oxo-2,5,9,12,15,18,21,24,27-nonaoxa-31-azapentatriacontan-35-yl)-5,8,13,18,23,26-hexaazatriacontane-1,30-dioyl)bis-(azanediyl))bis(4-hydroxy-3,1-phenylene))bis(4-((tert-butoxycarbonyl)amino)-2-methylpentanoate) (0.175 g, 0.0647 mmol) was dissolved in DCM (6 ml), followed by addition of TFA (2 ml). The reaction mixture was stirred at RT for 1h, diluted with toluene (8 ml), concentrated to afford (2S,2'S,4R,4′R)-5,5′-((((7S,15R,16S,24S)-15,16-bis(3-(2,5-dioxo-2,5-dihydro-1H-pyrrol-1-yl)propanamido)-6,9,14,17,22,25-hexaoxo-7,24-bis(30-oxo-2,5,9,12,15,18,21,24,27-nonaoxa-31-azapentatriacontan-35-yl)-5,8,13,18,23,26-hexaazatriacontane-1,30-dioyl)bis(azanediyl))bis(4-hydroxy-3,1-phenylene))bis(4-amino-2-methylpentanoic acid) (155 mg, 100% yield) for the next step without further purification. Then the compound in DMA (15 ml) was added perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (46 mg, 0.065 mmol) and DIPEA (10 ul, 0.0575 mmol). The reaction mixture was stirred overnight, concentrated and purified on HPLC with a gradient of MeCN/H₂O (10% MeCN to 70% MeCN in 45 min, C-18 column, 20 mm (d)×250 mm (1), 9 ml/min) to give the title product (105.3 mg, 48% yield). ESI: m/z: calcd for C₁₆₂H₂₆₃N₂₄O₅₀S₂ [M+H]⁺: 3408.81, found 3408.60.

### Example 260. Synthesis of (2S,4R)-methyl 4-hydroxypyrrolidine-2-carboxylate hydrochloric

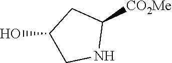

To a solution of trans-4-hydroxy-L-proline (15.0 g, 114.3 mmol) in dry methanol (250 mL) was added thionyl chloride (17 mL, 231 mmol) dropwise at 0 to 4° C. The resulting mixture was stirred for at r.t. overnight, concentrated, crystallized with EtOH/hexane to provide the title compound (18.0 g, 87% yield). ESI MS m/z 168.2 ([M+Na]⁺).

### Example 261. Synthesis of (2S,4R)-1-tert-butyl 2-methyl 4-hydroxypyrrolidine-1,2-dicarboxylate

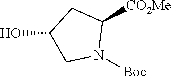 [SMILES: COC(=O)[C@@H]1C[C@@H](O)CN1C(=O)OC(C)(C)C]

To a solution of trans-4-hydroxy-L-proline methyl ester (18.0 g, 107.0 mmol) in the mixture of MeOH (150 ml) and sodium bicarbonate solution (2.0 M, 350 ml) was added Boc₂O (30.0 g, 137.6 mmol) in three portions in 4 h. After stirring for an additional 4 h, the reaction was concentrated to ˜350 ml and extracted with EtOAc (4×80 mL). The combined organic layers were washed with brine (100 mL), dried (MgSO₄), filtered, concentrated and purified by SiO₂ column chromatography (1:1 hexanes/EtOAc) to give the title compound (22.54 g, 86% yield). ESI MS m/z 268.2 ([M+Na]⁺).

### Example 262. Synthesis of (S)-1-tert-butyl 2-methyl 4-oxopyrrolidine-1,2-dicarboxylate

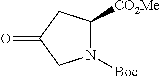 [SMILES: COC(=O)[C@@H]1CC(=O)CN1C(=O)OC(C)(C)C]

The title compound prepared through Dess-Martin oxidation was described in: Franco Manfre et al. J. Org. Chem. 1992, 57, 2060-2065. Alternatively Swern oxidation procedure is as following: To a solution of (COCl)₂ (13.0 ml, 74.38 mmol) in CH₂Cl₂ (350 ml) cooled to −78° C. was added dry DMSO (26.0 mL). The solution was stirred at −78° C. for 15 min and then (2S,4R)-1-tert-butyl 2-methyl 4-hydroxypyrrolidine-1,2-dicarboxylate (8.0 g, 32.63 mmol) in CH₂Cl₂ (100 ml) was added. After stirring at −78° C. for 2 h, triethylamine (50 ml, 180.3 mmol) was added dropwise, and the reaction solution was warmed to room temperature. The mixture was diluted with aq. NaH₂PO₄ solution (1.0 M, 400 ml) and phases separated. The aqueous layer was extracted with CH₂Cl₂ (2×60 ml). The organic layers were combined, dried over MgSO₄, filtered, concentrated and purified by SiO₂ column chromatography (7:3 hexanes/EtOAc) to give the title compound (6.73 g, 85% yield). ESI MS m/z 266.2 ([M+Na]⁺).

### Example 263. Synthesis of (S)-1-tert-butyl 2-methyl 4-methylenepyrrolidine-1,2-dicarboxylate

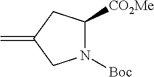 [SMILES: C=C1C[C@@H](C(=O)OC)N(C(=O)OC(C)(C)C)C1]

To a suspension of methyltriphenylphosphonium bromide (19.62 g, 55.11 mmol) in THE (150 mL) at 0° C. was added potassium-t-butoxide (6.20 g, 55.30 mmol) in anhydrous THE (80 mL). After stirring at 0° C. for 2 h, the resulting yellow ylide was added to a solution of (S)-1-tert-butyl 2-methyl 4-oxopyrrolidine-1,2-dicarboxylate (6.70 g, 27.55 mmol) in THE (40 mL). After stirring at r.t. for 1 h, the reaction mixture was concentrated, diluted with EtOAc (200 mL), washed with H₂O (150 mL), brine (150 mL), dried over MgSO₄, concentrated and purified on SiO₂ column chromatography (9:1 hexanes/EtOAc) to yield the title compound (5.77 g, 87% yield). EI MS m/z 264 ([M+Na]⁺).

### Example 264. Synthesis of (S)-methyl 4-methylenepyrrolidine-2-carboxylate hydrochloride

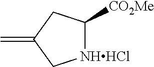

To a solution of (S)-1-tert-butyl 2-methyl 4-methylenepyrrolidine-1,2-dicarboxylate (5.70 g, 23.63 mmol) in EtOAc (40 ml) at 4° C. was added HCl (12 M, 10 ml). The mixture was stirred for 1 h, diluted with toluene (50 ml), concentrated, and crystallized with EtOH/hexane to yield the title compound as HCl salt (3.85 g, 92% yield). EI MS m/z 142.2 ([M+H]⁺).

### Example 265. Synthesis of (S)-tert-butyl 2-(hydroxymethyl)-4-methylenepyrrolidine-1-carboxylate

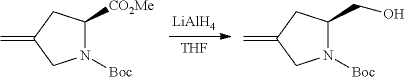 [SMILES: C=C1C[C@@H](C(=O)OC)N(C(=O)OC(C)(C)C)C1 ; "LiAlH4" ; C=C1C[C@@H](CO)N(C(=O)OC(C)(C)C)C1 ; "THF"]

To a solution of (S)-1-tert-butyl 2-methyl 4-methylenepyrrolidine-1,2-dicarboxylate. (5.20 g, 21.56 mmol) in anhydrous THE (100 mL) at 0° C. was added LiAlH₄ (15 ml, 2M in THF). After stirring at 0° C. for 4 h, the reaction was quenched by addition of methanol (5 ml) and water (20 ml). The reaction mixture was neutralized with 1 M HCl to pH 7, diluted with EtOAc (80 ml), filtered through Celite, separated and the aqueous layer was extracted with EtOAc. The organic layers were combined, dried over Na₂SO₄, concentrated and purified on SiO₂ column chromatography (1:5 EtOAc/DCM) to yield the title compound (3.77 g, 82% yield). EI MS m/z 236.40 ([M+Na]⁺).

### Example 266. Synthesis of (S)-(4-methylenepyrrolidin-2-yl)methanol, hydrochloride salt

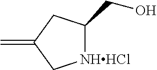

To a solution of (S)-tert-butyl 2-(hydroxymethyl)-4-methylenepyrrolidine-1-carboxylate (3.70 g, 17.36 mmol) in EtOAc (30 ml) at 4° C. was added HCl (12 M, 10 ml). The mixture was stirred for 1 h, diluted with toluene (50 ml), concentrated, and crystallized with EtOH/hexane to yield the title compound as HCl salt (2.43 g, 94% yield). EI MS m/z 115.1 ([M+H]⁺).

### Example 267. Synthesis of 4-(benzyloxy)-3-methoxybenzoic acid

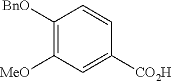 [SMILES: COc1cc(C(=O)O)ccc1OCc1ccccc1]

To a mixture of 4-hydroxy-3-methoxybenzoic acid (50.0 g, 297.5 mmol) in ethanol (350 ml) and aq. NaOH solution (2.0 M, 350 ml) was added BnBr (140.0 g, 823.5 mmol). The mixture was stirred at 65° C. for 8 h, concentrated, co-evaporated with water (2×400 ml) and concentrated to ˜400 ml, acidified to pH 3.0 with 6 N HCl. The solid was collected by filtration, crystallized with EtOH, dried at 45° C. under vacuum to afford the title compound (63.6 g, 83% yield). ESI MS m/z 281.2 ([M+Na]⁺).

### Example 268. Synthesis of 4-(benzyloxy)-5-methoxy-2-nitrobenzoic Acid

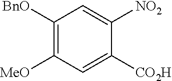 [SMILES: COc1cc(C(=O)O)c([N+](=O)[O-])cc1OCc1ccccc1]

To a solution of 4-(benzyloxy)-3-methoxybenzoic acid (63.5 g, 246.0 mmol) in CH₂Cl₂ (400 ml) and HOAc (100 ml) was added HNO₃ (fuming, 25.0 ml, 528.5 mmol). The mixture was stirred for 6 h, concentrated, crystallized with EtOH, dried at 40° C. under vacuum to afford the title compound (63.3 g, 85% yield). ESI MS m/z 326.1 ([M+Na]⁺).

### Example 269. Synthesis of (S)-(4-(benzyloxy)-5-methoxy-2-nitrophenyl)(2-(hydroxymethyl)-4-methylenepyrrolidin-1-yl)methanone

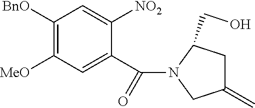 [SMILES: C=C1C[C@@H](CO)N(C(=O)c2cc(OC)c(OCc3ccccc3)cc2[N+](=O)[O-])C1]

A catalytic amount of DMF (30 μl) was added to a solution of 4-(benzyloxy)-5-methoxy-2-nitrobenzoic acid (2.70 g, 8.91 mmol) and oxalyl chloride (2.0 mL, 22.50 mmol) in anhydrous CH₂Cl₂ (70 mL) and the resulting mixture was stirred at room temperature for 2 h. Excess CH₂C12 and oxalyl chloride was removed with rotavap. The acetyl chloride was re-suspended in fresh CH₂Cl₂ (70 mL) and was added slowly to a pre-mixed solution of (S)-(4-methylenepyrrolidin-2-yl)methanol, hydrochloride salt (1.32 g, 8.91 mmol) and Et₃N (6 mL) in CH₂Cl₂ at 0° C. under N₂ atmosphere. The reaction mixture was allowed to warm to r.t. and stirring was continued for 8 h. After removal of CH₂Cl₂ and Et₃N, the residue was partitioned between H₂O and EtOAc (70/70 mL). The aqueous layer was further extracted with EtOAc (2×60 mL). The combined organic layers were washed with brine (40 mL), dried (MgSO₄) and concentrated. Purification of the residue with flash chromatography (silica gel, 2:8 hexanes/EtOAc) yielded the title compound (2.80 g, 79% yield). EI MS m/z 421.2 ([M+Na]⁺).

### Example 270. Synthesis of (S)-(4-(benzyloxy)-5-methoxy-2-nitrophenyl)(2-(((tert-butyldimethylsilyl)oxy)methyl)-4-methylenepyrrolidin-1-yl)methanone

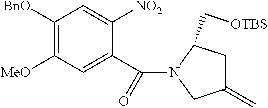 [SMILES: C=C1C[C@@H](CO[Si](C)(C)C(C)(C)C)N(C(=O)c2cc(OC)c(OCc3ccccc3)cc2[N+](=O)[O-])C1]

(S)-(4-(Benzyloxy)-5-methoxy-2-nitrophenyl)(2-(hydroxymethyl)-4-methylenepyrrolidin-1-yl)methanone (2.78 g, 8.52 mmol) in the mixture of DCM (10 ml) and pyridine (10 ml) was added tert-butylchlorodimethylsilane (2.50 g, 16.66 mmol). The mixture was stirred for overnight, concentrated and purified on SiO₂ column eluted with EtOAc/CH₂Cl₂ (1:6) to afford the title compound (3.62 g, 83% yield, ˜95% pure). MS ESI m/z calcd for C27H₃₇N₂O₆Si [M+H]⁺ 513.23, found 513.65.

### Example 271. Synthesis of (S)-(4-hydroxy-5-methoxy-2-nitrophenyl)(2-(hydroxymethyl)-4-methylenepyrrolidin-1-yl)methanone

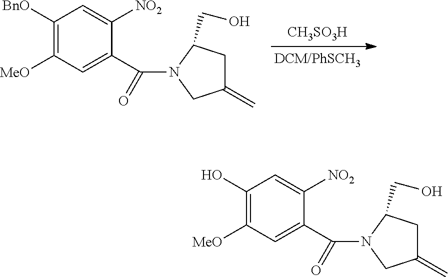

(S)-(4-(Benzyloxy)-5-methoxy-2-nitrophenyl)(2-(hydroxymethyl)-4-methylenepyrrolidin-1-yl)methanone (2.80 g, 7.03 mmol) in the mixture of DCM (30 ml) and CH₃SO₃H (8 ml) was added PhSCH₃ (2.00 g, 14.06 mmol). The mixture was stirred for 0.5 h, diluted with DCM (40 ml), neutralized with carefully addition of 0.1 M Na₂CO₃ solution. The mixture was separated and the aqueous solution was extracted with DCM (2×10 ml). The organic layers were combined, dried over Na₂SO₄, concentrated and purified on SiO₂ column eluted with MeOH/CH₂Cl₂ (1:15 to 1:6) to afford the title compound (1.84 g, 85% yield, ˜95% pure). MS ESI m/z calcd for C₁₄H₁₇N₂O₆ [M+H]⁺ 309.10, found 309.30.

### Example 272. Synthesis of (S)-((pentane-1,5-diylbis(oxy))bis(5-methoxy-2-nitro-4,1-phenylene))bis(((S)-2-(hydroxymethyl)-4-methylenepyrrolidin-1-yl)methanone)

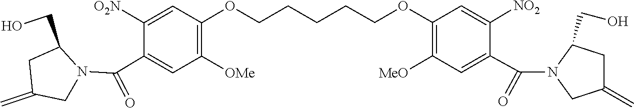

(S)-(4-hydroxy-5-methoxy-2-nitrophenyl)(2-(hydroxymethyl)-4-methylenepyrrolidin-1-yl)methanone (0.801 g, 2.60 mmol) in butanone (10 ml) was added Cs₂CO₃, (2.50 g, 7.67 mmol), followed by addition of 1,5-diiodopentane (415 mmol, 1.28 mmol). The mixture was stirred for 26 h, concentrated and purified on SiO₂ column eluted with MeOH/CH₂Cl₂ (1:15 to 1:5) to afford the title compound (0.675 g, 77% yield, ˜95% pure). MS ESI m/z calcd for C₃₃H₄₁N₄O₁₂ [M+H]⁺ 685.26, found 685.60.

### Example 273. Synthesis of (S)-((pentane-1,5-diylbis(oxy))bis(2-amino-5-methoxy-4,1-phenylene))bis(((S-2-(hydroxymethyl)-4-methylenepyrrolidin-1-yl)methanone)

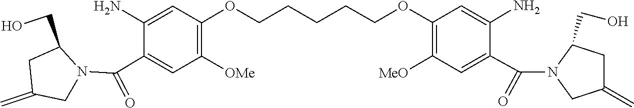

(S)-((pentane-1,5-diylbis(oxy))bis(5-methoxy-2-nitro-4,1-phenylene))bis(((S)-2-(hydroxymethyl)-4-methylenepyrrolidin-1-yl)methanone) (0.670 g, 0.98 mmol) in CH₃OH (10 ml) was added Na₂S₂O₄ (1.01 g, 5.80 mmol) in H₂O (8 ml). The mixture was stirred at room temperature for 30 h. The reaction mixture was evaporated and co-evaporated with DMA (2×10 mL) and EtOH (2×10 ml)under high vacuum to dryness to afford the title compound (total weight 1.63 g) containing inorganic salts which was used directly for the next step reaction (without further separation). EIMS m/z 647.32 ([M+Na]⁺).

### Example 274. Synthesis of C-01 (a PBD Dimer Analog Having a Bis-Linker)

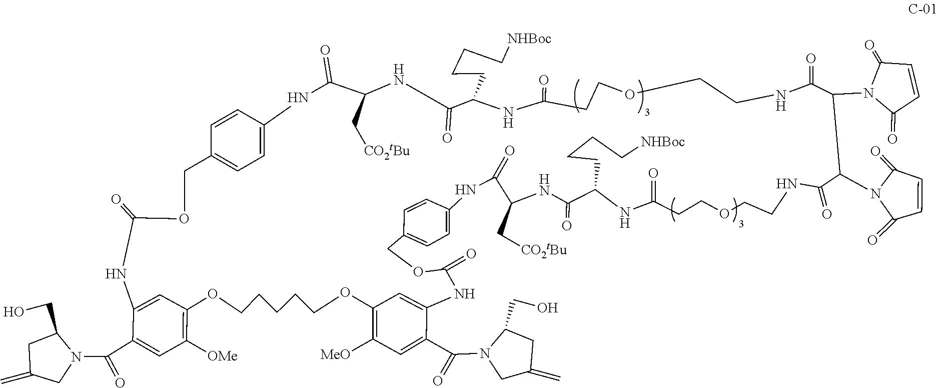

(3S,6S,39S,42S)-di-tert-butyl 6,39-bis(4-((tert-butoxycarbonyl)amino)butyl)-22,23-bis(2,5-dioxo-2,5-dihydro-1H-pyrrol-1-yl)-3,42-bis((4-(hydroxymethyl)phenyl)carbamoyl)-5,8,21,24,37,40-hexaoxo-11,14,17,28,31,34-hexaoxa-4,7,20,25,38,41-hexaazatetratetracontane-1,44-dioate (0.840 g, 0.488 mmol) in THE (8 mL) containing pyridine (0.100 ml, 1.24 mmol) at 0° C. was added dropwise of a solution of triphosgene (0.290 mg, 0.977 mmol) in THE (3.0 mL). The reaction mixture was stirred at 0° C. for 15 min then was used directly in the next step.

(S)-((pentane-1,5-diylbis(oxy))bis(2-amino-5-methoxy-4,1-phenylene))bis(((S)-2-(hydroxymethyl)-4-methylenepyrrolidin-1-yl)methanone) containing inorganic salts (0.842 mg, −0.49 mmol) was suspended in EtOH (10 ml) at 0° C. was added the trichloride in THE prepared above. The mixture was stirred at 0° C. for 4 h, then warmed to RT for 1 h, concentrated, and purified by reverse phase HPLC (250 (L) mm x 10(d) mm, C₁₈ column, 10-80% acetonitrile/water in 40 min, v=8 ml/min) to afford the C-01 compound (561.1 mg, 48% yield in three steps). ESI MS m/z: calcd for C₁₁₇H₁₆₃N₁₆O₃₈ [M+H]⁺ 2400.12, found 2400.90.

### Example 275. Synthesis of C-02 (a PBD Dimer Analog Having a Bis-Linker)

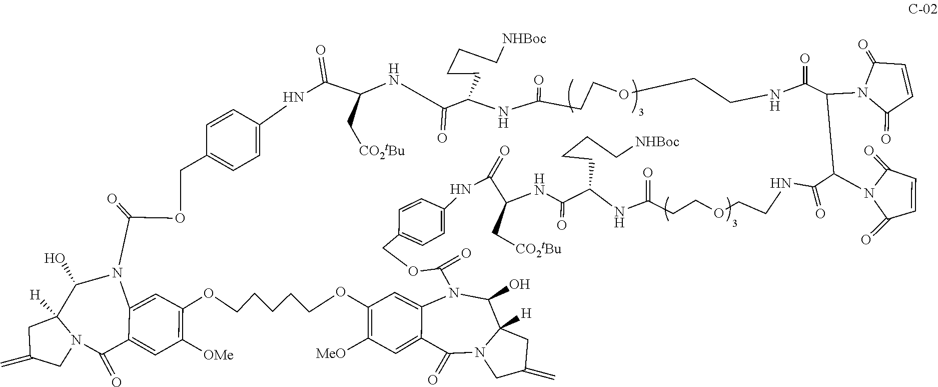

Dess-Martin periodinane (138.0 mg, 0.329 mmol) was added to a solution of compound C-01 (132.0 mg, 0.055 mmol) in DCM (5.0 mL) at 0° C. The reaction mixture was warmed to RT and was stirred for 2 h. A saturated solution of NaHCO₃/Na₂SO₃ (5.0 mL/5.0 mL) was then added and the mixture was extracted with DCM (3×25 mL). The combined organic layers were washed with NaHCO₃/Na₂SO₃ (5.0 mL/5.0 mL), brine (10 mL), dried over Na₂SO₄, filtered, concentrated and purified by reverse phase HPLC (250 (L) mm x 10(d) mm, C₁₈ column, 10-80% acetonitrile/water in 40 min, v=8 ml/min) to afford the title compound (103.1 mg, 78% yield) as a foam. ESI MS m/z: calcd for C₁₁₇H₁₅₈N₁₆O₃₈ [M+H]⁺ 2396.09, found 2396.65.

### Example 276. Synthesis of C-03 (a PBD Dimer Analog Having a Bis-Linker)

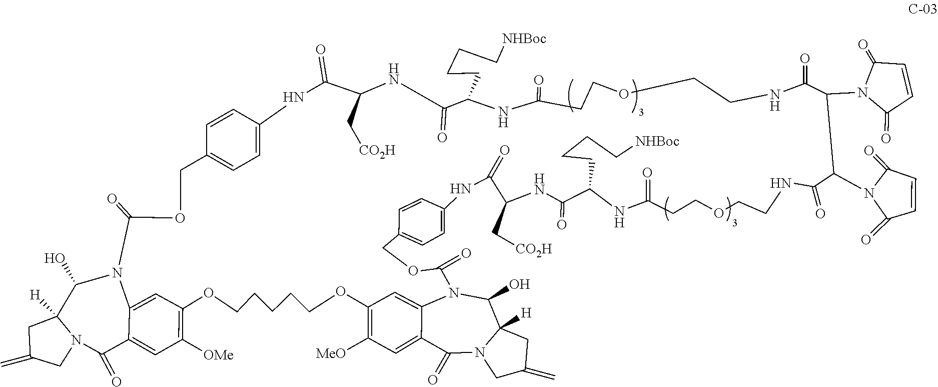

C-02 compound (55.0 mg, 0.023 mmol) was dissolved in DCM (3 ml), followed by addition of TFA (3 ml). The reaction mixture was stirred at RT for 2 h, then concentrated, and co-evaporated with DCM/toluene to dryness to afford the crude product C-3 (48.0 mg, 100% yield, 92% pure by HPLC) which was further purified by reverse phase HPLC (250 (L) mm x 10(d) mm, C₁₈ column, 5-60% acetonitrile/water in 40 min, v=8 ml/min) to afford the pure product C-03 (42.1 mg, 88% yield, 96% pure) as a foam. ESI MS m/z: calcd for C₉₉H₁₂₆N₁₆O₃₄ [M+H]⁺ 2083.86, found 2084.35.

### Example 277. Synthesis of C-04 (a PBD Dimer Analog Having a Bis-Linker)

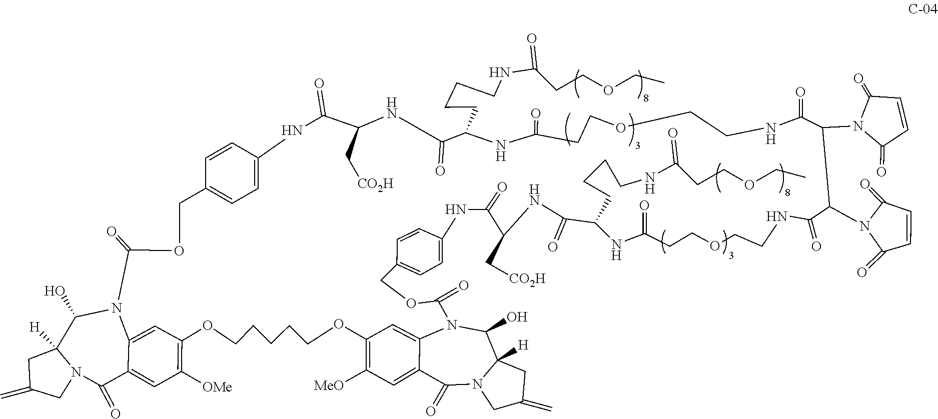

C-03 compound (35.0 mg, 0.017 mmol) was dissolved in a mixture solution of THF (3 ml) and 0.1 M, NaH₂PO₄ (3 ml), pH 7.5, followed by addition of N-succinimidyl 2,5,8,11,14,17,20,23-octaoxahexacosan-26-oate (43.0 mg, 0.084 mmol) in 4 portions in 2 h. The reaction mixture was then continued to stir at RT for 4 h, and co-evaporated with DMF (10 ml) to dryness to afford the crude product C-4 which was further purified by reverse phase HPLC (250 (L) mm x 20(d) mm, C₁₈ column, 20-60% acetonitrile/water in 40 min, v=8 ml/min) to afford the pure product C-04 (39.4 mg, 81% yield, 96% pure) as a foam. ESI MS m/z: calcd for C₁₃₅H₁₉₅N₁₆O₅₂ [M+H]⁺ 2872.30, found 2871.65.

### Example 278. Synthesis of C-05 (a PBD Dimer Analog Having a Bis-Linker)

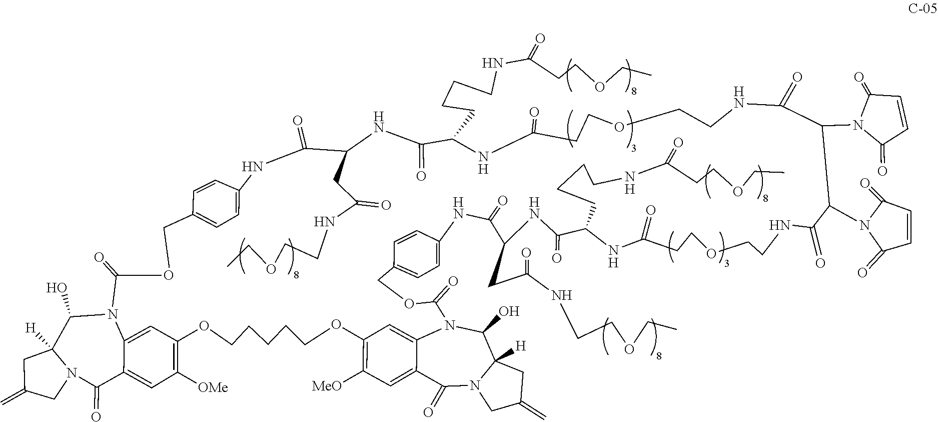

To a solution of C-04 compound (35.0 mg, 0.012 mmol) and 2,5,8,11,14,17,20,23-octaoxapentacosan-25-amine (15.1 mg, 0.0394 mmol) in dry DMA (2 ml) was added EDC (30.0 mg, 0.156 mmol). The reaction mixture was stirred at RT for 14 h, concentrated, purified by reverse phase HPLC (250 (L) mm x 20(d) mm, C₁₈ column, 20-60% acetonitrile/water in 40 min, v=8 ml/min) to afford the pure product C-05 (31.2 mg, 77% yield, 97% pure by HPLC) as a foam. ESI MS m/z: calcd for C₁₆₁H₂₄₉N₁₈O₆₂ [M+H]⁺ 3426.68, found 3427.21.

### Example 279. Synthesis of (S)-methyl 1-(4-(benzyloxy)-5-methoxy-2-nitrobenzoyl)-4-methylenepyrrolidine-2-carboxylate

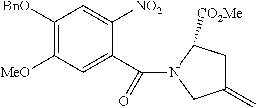 [SMILES: C=C1C[C@@H](C(=O)OC)N(C(=O)c2cc(OC)c(OCc3ccccc3)cc2[N+](=O)[O-])C1]

A catalytic amount of DMF (30 μl) was added to a solution of 4-(benzyloxy)-5-methoxy-2-nitrobenzoic acid (2.70 g, 8.91 mmol) and oxalyl chloride (2.0 mL, 22.50 mmol) in anhydrous CH₂Cl₂ (70 mL) and the resulting mixture was stirred at room temperature for 2 h. Excess CH₂Cl₂ and oxalyl chloride was removed with rotavap. The acetyl chloride was re-suspended in fresh CH₂Cl₂ (70 mL) and was added slowly to a pre-mixed solution of (S)-methyl 4-methylenepyrrolidine-2-carboxylate hydrochloride (1.58 g, 8.91 mmol) and Et₃N (6 mL) in CH₂Cl₂ at 0° C. under N₂ atmosphere. The reaction mixture was allowed to warm to r.t. and stirring was continued for 8 h. After removal of CH₂Cl₂ and Et₃N, the residue was partitioned between H₂O and EtOAc (70/70 mL). The aqueous layer was further extracted with EtOAc (2×60 mL). The combined organic layers were washed with brine (40 mL), dried (MgSO₄) and concentrated. Purification of the residue with flash chromatography (silica gel, 2:8 hexanes/EtOAc) yielded the title compound (2.88 g, 76% yield). EI MS m/z 449.1 ([M+Na]⁺).

### Example 280. Synthesis of (S)-1-(4-(benzyloxy)-5-methoxy-2-nitrobenzoyl)-4-methylenepyrrolidine-2-carbaldehyde

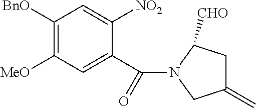 [SMILES: C=C1C[C@@H](C=O)N(C(=O)c2cc(OC)c(OCc3ccccc3)cc2[N+](=O)[O-])C1]

To a vigorously stirred solution of (S)-methyl 1-(4-(benzyloxy)-5-methoxy-2-nitro benzoyl)-4-methylenepyrrolidine-2-carboxylate (2.80 g, 6.57 mmol) in anhydrous CH₂Cl₂ (60 mL) was added DIBAL-H (1N in CH₂Cl₂, 10 mL) dropwise at −78° C. under N₂ atmosphere. After the mixture was stirred for an additional 90 min, excess reagent was decomposed by addition of 2 ml of methanol, followed by 5% HCl (10 mL). The resulting mixture was allowed to warm to 0° C. Layers were separated and the aqueous layer was further extracted with CH₂Cl₂ (3×50 mL). Combined organic layers were washed with brine, dried (MgSO₄) and concentrated. Purification of the residue with flash chromatography (silica gel, 95:5 CHCl₃/MeOH) yielded the title compound (2.19 g, 84% yield). EIMS m/z 419.1 ([M+Na]⁺).

### Example 281. Synthesis of (S)-8-(benzyloxy)-7-methoxy-2-methylene-2,3-dihydro-1H-benzo[e]-pyrrolo[1,2-a]azepin-5(11aH)-one

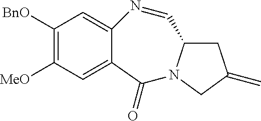 [SMILES: C=C1C[C@H]2C=Nc3cc(OCc4ccccc4)c(OC)cc3C(=O)N2C1]

A mixture of (S)-1-(4-(benzyloxy)-5-methoxy-2-nitrobenzoyl)-4-methylenepyrrolidine-2-carbaldehyde (2.18 g, 5.50 mmol) and Na₂S₂O₄ (8.0 g, 45.97 mmol) in THE (60 ml) and H₂O (40 ml) was stirred at room temperature for 20 h. Solvents were removed under high vacuum. The residue was re-suspended in MeOH (60 mL), and HCl (6M) was added dropwise until pH ˜ 2 was reached. The resulting mixture was stirred at r.t. for 1 h. The reaction was worked-up by removing most of MeOH, then diluted with EtOAc (100 mL). The EtOAc solution was washed with sat. NaHCO₃, brine, dried (MgSO₄), and concentrated. Purification of the residue with flash chromatography (silica gel, 97:3 CHCl₃/MeOH) yielded the title compound (1.52 g, 80%). EIMS m/z 372.1 ([M+Na]⁺).

### Example 282. Synthesis of (S)-8-hydroxy-7-methoxy-2-methylene-2,3-dihydro-1H-benzo[e]-pyrrolo[1,2-a]azepin-5(11aH)-one

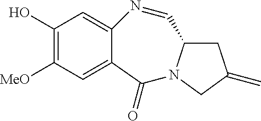 [SMILES: C=C1C[C@H]2C=Nc3cc(O)c(OC)cc3C(=O)N2C1]

To a solution of (S)-8-(benzyloxy)-7-methoxy-2-methylene-2,3-dihydro-1H-benzo[e]-pyrrolo[1,2-a]azepin-5(11aH)-one (1.50 g, 4.32 mmol) in 70 ml of CH₂C2was added 25 ml of CH₃SO₃H at 0° C. The mixture was stirred at 0° C. for 10 min then r.t. for 2 h, diluted with CH₂Cl₂, pH adjusted with cold 1.0 N NaHCO₃ to 4 and filtered. The aqueous layer was extracted with CH₂Cl₂ (3×60 ml). The organic layers were combined, dried over Na₂SO₄, filtered, evaporated and purified on SiO₂ column chromatography (CH₃OH/CH₂Cl₂ 1:15) to afford 811 mg (73% yield) of the title product. EIMS m/z 281.1 ([M+Na]⁺).

### Example 283. Synthesis of (11aS,11a'S)-8,8′-(pentane-1,5-diylbis(oxy))bis(7-methoxy-2-methylene-2,3-dihydro-1H-benzo[e]pyrrolo[1,2-a][1,4]diazepin-5(11aH)-one)

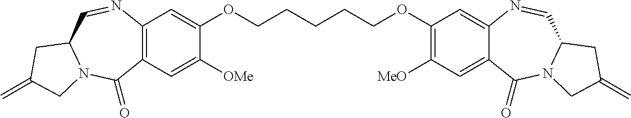

To a stirred suspended solution of Cs₂CO₃ (0.761 g, 2.33 mmol) in butanone (8 ml) were added (S)-8-hydroxy-7-methoxy-2-methylene-2,3-dihydro-1H-benzo[e]-pyrrolo[1,2-a]azepin-5(11aH)-one (401 mg, 1.55 mmol) and 1,5-diiodopentane (240 mg, 0.740 mmol). The mixture was stirred at r.t. overnight, concentrated, and purified on SiO₂ chromatography (EtOAc/CH₂Cl₂ 1:10) to afford 337 mg (78% yield) of the title product. EIMS m/z 607.2 ([M+Na]⁺).

### Example 284. Synthesis of (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-methylene-2,3-dihydro-1H-benzo[e]pyrrolo[1,2-a][1,4]diazepin-5(11aH)-one

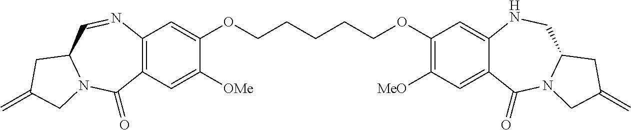

To a solution of (11aS,11a'S)-8,8′-(pentane-1,5-diylbis(oxy))bis(7-methoxy-2-methylene-2,3-dihydro-1H-benzo[e]pyrrolo[1,2-a][1,4]diazepin-5(11aH)-one) (150 mg, 0.256 mmol) in anhydrous dichloromethane (1 mL) and absolute ethanol (1.5 mL) was added sodium borohydride in methoxyethyl ether (85 μl, 0.5 M, 0.042 mmol) at 0° C. The ice bath was removed after 5 minutes and the mixture was stirred at room temperature for 3 hours, then cooled to 0° C., quenched with saturated ammonium chloride, diluted with dichloromethane, and phases separated. The organic layer was washed with brine, dried over anhydrous Na₂SO₄, filtered through Celite and concentrated. The residue was purified by reverse phase HPLC (C₁₈ column, acetonitrile/water). The corresponding fractions were extracted with dichloromethane and concentrated to afford the title compound (64.7 mg, 43%), MS m/z 609.2 ([M+Na]⁺), 625.3 ([M+K]⁺) and 627.2 ([M+Na+H₂O]⁺); the fully reduced compound was obtained (16.5 mg, 11%), MS m/z 611.2 ([M+Na]⁺), 627.2 ([M+K]⁺), 629.2 ([M+Na+H₂O]⁺); and the unreacted starting material was also recovered (10.2 mg, 7%), MS m/z 607.2 ([M+Na]⁺), 625.2 ([M+Na+H₂O]⁺).

### Example 285. Synthesis of (S)-8-((5-(((S)-10-(3-(2-(2-azidoethoxy)ethoxy) propanoyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-2,3-dihydro-1H-benzo[e]pyrrolo[1,2-a][1,4]diazepin-5(11aH)-one

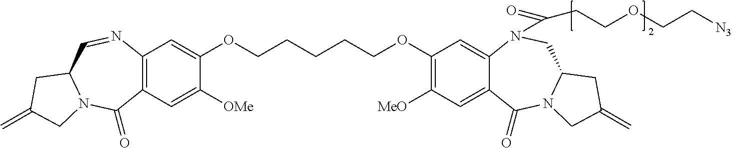

To the mixture of (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-methylene-2,3-dihydro-1H-benzo[e]pyrrolo[1,2-a][1,4]diazepin-5(11aH)-one (60.0 mg, 0.102 mmol) and 2,5-dioxopyrrolidin-1-yl 3-(2-(2-azidoethoxy)ethoxy)propanoate (40.5 mg, 0.134 mmol) in dichloromethane (5 ml) was added EDC (100.5 mg, 0.520 mmol). The mixture was stirred at r.t. overnight, concentrated and purified on SiO₂ column chromatography (EtOAc/CH₂Cl₂, 1:6) to afford 63.1 mg (81% yield) of the title product. ESI MS m/z C₄₀H₅₀N₇O₉ [M+H]⁺, cacld. 772.36, found 772.30.

### Example 286. Synthesis of (S)-8-((5-(((S)-10-(3-(2-(2-aminoethoxy)ethoxy) propanoyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-2,3-dihydro-1H-benzo[e]pyrrolo[1,2-a][1,4]diazepin-5(11aH)-one

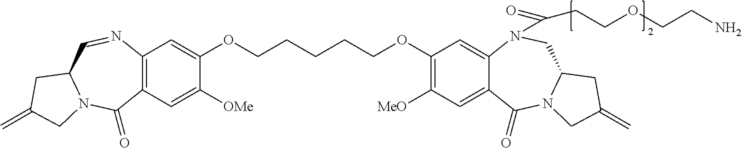

To a solution of (S)-8-((5-(((S)-10-(3-(2-(2-azidoethoxy)ethoxy) propanoyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-2,3-dihydro-1H-benzo[e]pyrrolo[1,2-a][1,4]diazepin-5(11aH)-one (60 mg, 0.078 mmol) in the mixture of THF (5 ml) and NaH₂PO₄ buffer solution (pH 7.5, 1.0 M, 0.7 ml) was added PPh₃ (70 mg, 0.267 mmol). The mixture was stirred at r.t. overnight, concentrated and purified on C₁₈ preparative HPLC, eluted with water/CH₃CN (from 90% water to 35% water in 35 min) to afford 45.1 mg (79% yield) of the title product after drying under high vacuum. ESI MS m/z C₄₀H₅₂N₅O₉ [M+H]⁺, cacld. 746.37, found 746.50.

### Example 287. Synthesis of (S)—N-(2-((S)-8-((5-(((11S,11aS)-10-((S)-15-azido-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)-oxy)-7-methoxy-2-methylene-5-oxo-2,3,11,11a-tetrahydro-1H-benzo[e]pyrrolo[1,2-a][1,4]diazepin-10(5H)-yl)-2-oxoethyl)-2-(3-(2-(2-azidoethoxy)ethoxy)propanamido)-3-methylbutanamide

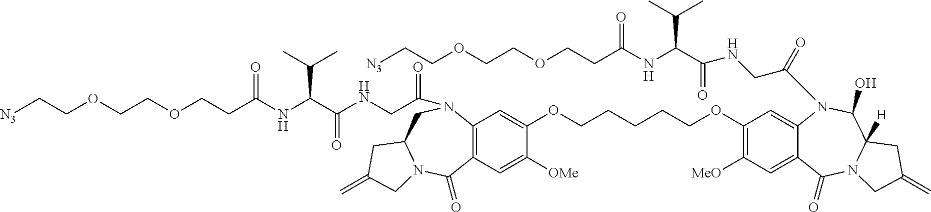 [SMILES: C=C1C[C@H]2CN(C(=O)CNC(=O)[C@@H](NC(=O)CCOCCOCCN=[N+]=[N-])C(C)C)c3cc(OCCCCCOc4cc5c(cc4OC)C(=O)N4CC(=C)C[C@H]4[C@H](O)N5C(=O)CNC(=O)[C@@H](NC(=O)CCOCCOCCN=[N+]=[N-])C(C)C)c(OC)cc3C(=O)N2C1]

To the mixture of (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-methylene-2,3-dihydro-1H-benzo[e]pyrrolo[1,2-a][1,4]diazepin-5(11aH)-one (60.0 mg, 0.102 mmol) and (S)-15-azido-5-isopropyl-4,7-dioxo-10,13-dioxa-3,6-diazapentadecan-1-oic acid (90.2 mg, 0.25 mmol) in DMA (8 ml) was added BrOP (240.2 mg, 0.618 mmol). The mixture was stirred at r.t. overnight, concentrated and purified on SiO₂ column chromatography (CH₃OH/CH₂Cl₂, 1:10 to 1:5) to afford 97.1 mg (74% yield) of the title product. ESI MS m/z C₆₁H₈₇N₁₄O₁₇ [M+H]⁺, cacld. 1287.63, found 1287.95.

### Example 288. Synthesis of (S)—N-(2-((S)-8-((5-(((11S,11aS)-10-((S)-15-amino-5-iso-propyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,11,11a-tetrahydro-1H-benzo[e]-pyrrolo[1,2-a][1,4]diazepin-10(5H)-yl)-2-oxoethyl)-2-(3-(2-(2-aminoethoxy)ethoxy)-propanamido)-3-methylbutanamide (C-06)

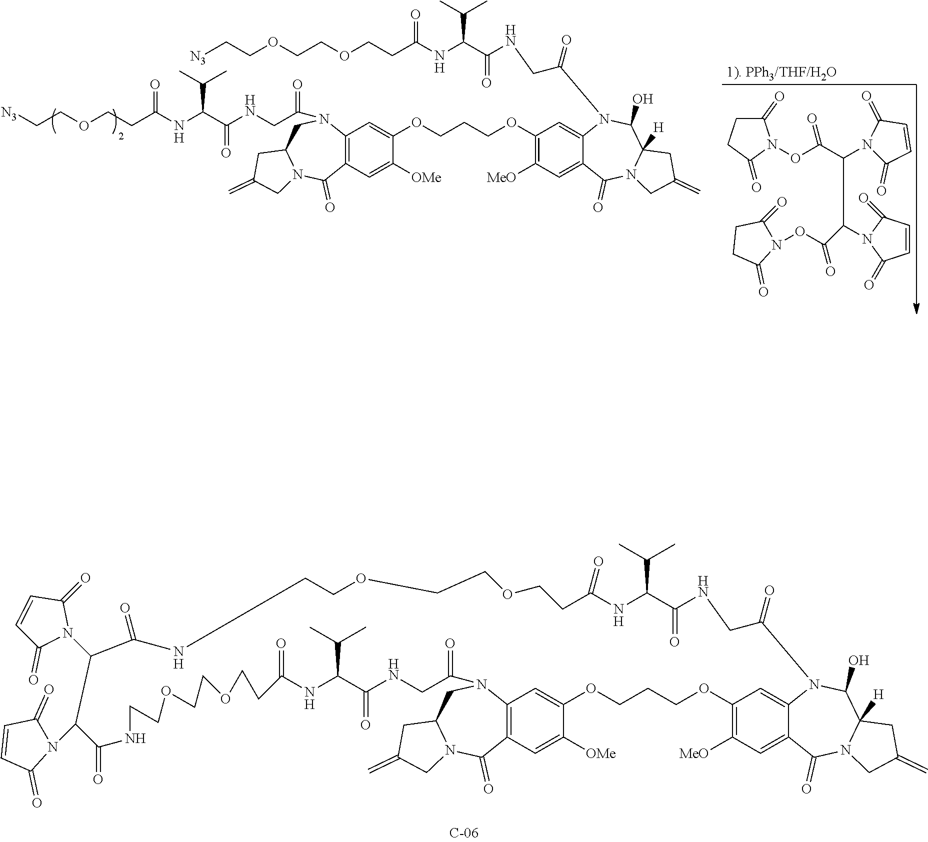

To a solution of (S)—N-(2-((S)-8-((5-(((11S,11aS)-10-((S)-15-azido-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)-oxy)-7-methoxy-2-methylene-5-oxo-2,3,11,11a-tetrahydro-1H-benzo[e]pyrrolo[1,2-a][1,4]diazepin-10(5H)-yl)-2-oxoethyl)-2-(3-(2-(2-azidoethoxy)ethoxy)propanamido)-3-methylbutanamide (85 mg, 0.066 mmol) in the mixture of THF (5 ml) and NaH₂PO₄ buffer solution (pH 7.5, 1.0 M, 0.7 ml) was added PPh₃ (100 mg, 0.381 mmol). The mixture was stirred at r.t. overnight. After confirmed by LC-MS to form (S)—N-(2-((S)-8-((5-(((11S,11aS)-10-((S)-15-amino-5-isopropyl-4,7-dioxo-10,13-dioxa-3,6-diazapentadecan-1-oyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-7-methoxy-2-methylene-5-oxo-2,3,11,11a-tetrahydro-1H-benzo[e]pyrrolo[1,2-a][1,4]diazepin-10(5H)-yl)-2-oxoethyl)-2-(3-(2-(2-aminoethoxy)ethoxy)propanamido)-3-methylbutanamide (ESI MS m/z C₆₁H₉₀N₁₀O₁₇ [M+Na]⁺, cacld. 1257.66, found 1257.90), bis(2,5-dioxopyrrolidin-1-yl) 2,3-bis(2,5-dioxo-2,5-dihydro-1H-pyrrol-1-yl)succinate (33 mg, 0.066 mmol) was added. The mixture was continued to stir for 4 h, concentrated and purified on C₁₈ preparative HPLC, eluted with water/CH₃CN (from 90% water to 30% water in 35 min) to afford 40.1 mg (40% yield) of the title product C-4 after drying under high vacuum. ESI MS m/z C₇₃H₉₅N₁₂O₂₃ [M+H]⁺, cacld. 1507.66, found 1507.90.

### Example 289. Synthesis of 4,4′-(pentane-1,5-diylbis(oxy))bis(3-methoxybenzoic acid)

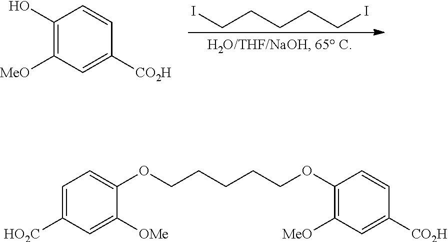 [SMILES: COc1cc(C(=O)O)ccc1O ; ICCCCCI ; COc1cc(C(=O)O)ccc1OCCCCCOc1ccc(C(=O)O)cc1OC]

A solution of diiodopropane (19.0 g, 58.6 mmol) in THE (75 mL) was added dropwise over a period of 4 hours to a vigorously stirred solution of vanillic acid (20.0 g, 119 mmol) in THE (150 mL) and aqueous NaOH (340 mL) at 65° C. in the absence of light (foil-wrapped flask). After heating at reflux for 48 hours in the dark, the solution was cooled and the THF removed by evaporation in vacuo. The residue was extracted with EA, The aqueous layer was separated and acidified to pH 2 with conc. HCl. The resultant precipitate collected by filtration, washed, dried and recrystallised from glacial acetic acid to afford the corresponding bis-carboxylic acid (14.0 g, 34.7 mmol). White solid, yield (60%).

### Example 290. Synthesis of4,4′-(pentane-1,5-diylbis(oxy))bis(5-methoxy-2-nitrobenzoic acid)

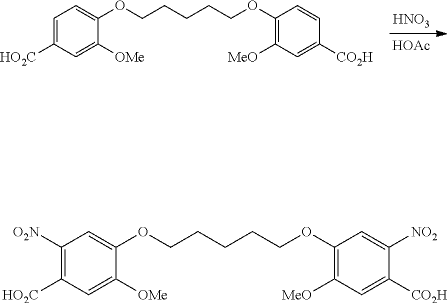 [SMILES: COc1cc(C(=O)O)ccc1OCCCCCOc1ccc(C(=O)O)cc1OC ; "HNO3" ; COc1cc(C(=O)O)c([N+](=O)[O-])cc1OCCCCCOc1cc([N+](=O)[O-])c(C(=O)O)cc1OC ; "HOAc"]

To a suspension of 4,4′-(pentane-1,5-diylbis(oxy))bis(3-methoxybenzoic acid) (18.0 g, 66.8 mmol) in HOAc (80 mL, 1800 mmol) was added HNO₃ (80 mL, 1778 mmol) dropwise at room temperature. After 2 h of stirring, the mixture was poured into 100 g ice and extracted with EA (2×200 mL). The organic layer was separated and washed with H₂O (2×100 mL), then 4N NaOH (400 mL) was added. After extracted with EA (2×100 mL), the basic aqueous layer was separated and acidified to pH 2 with conc. HCl. The mixture was extracted with EA (2×250 mL). The combined organic extract was washed with brine, dried, filtered and concentrated. The residue was purified by flash chromatography (DCM/MeOH=4/1) to give 4,4′-(pentane-1,5-diylbis(oxy))bis(5-methoxy-2-nitrobenzoic acid) (6.1 g, 12.3 mmol) as a pale yellow solid in 18% yield. R_{f} 0.3 (DCM/MeOH=3/1)

### Example 291. Synthesis of (S)-((pentane-1,5-diylbis(oxy))bis(5-methoxy-2-nitro-4,1-phenylene))bis(((S)-2-(hydroxymethyl)pyrrolidin-1-yl)methanone)

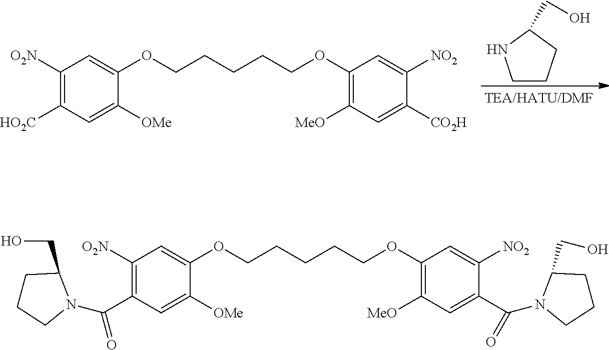 [SMILES: COc1cc(C(=O)O)c([N+](=O)[O-])cc1OCCCCCOc1cc([N+](=O)[O-])c(C(=O)O)cc1OC ; OC[C@@H]1CCCN1 ; COc1cc(C(=O)N2CCC[C@H]2CO)c([N+](=O)[O-])cc1OCCCCCOc1cc([N+](=O)[O-])c(C(=O)N2CCC[C@H]2CO)cc1OC]

To a solution of 4,4′-(pentane-1,5-diylbis(oxy))bis(5-methoxy-2-nitrobenzoic acid) (5.0 g, 10.0 mmol) and L-(+)-Prolinol (2.25 g, 22.3 mmol) in DMF (100 mL) was added TEA (4.0 g) at room temperature. After 10 min of stirring, HATU (10.77 g, 28.3 mmol) was added. The mixture was stirred at room temperature overnight. After completion of conversion, the mixture was diluted with H₂O (100 mL) and extracted with EA (2×100 mL) and DCM (2×50 mL), the combined organic extract was washed with brine, dried, filtered and concentrated. The residue was purified by chromatography (DCM/MeOH=15/1) to give (S)-((pentane-1,5-diylbis(oxy))bis(5-methoxy-2-nitro-4,1-phenylene))bis(((S)-2-(hydroxymethyl)pyrrolidin-1-yl)methanone) (6.0 g, 9.1 mmol) as a white foam in 91% yield.

### Example 292. Synthesis of (S)-((pentane-1,5-diylbis(oxy))bis(2-amino-5-methoxy-4,1-phenylene))bis(((S)-2-(hydroxymethyl)pyrrolidin-1-yl)methanone)

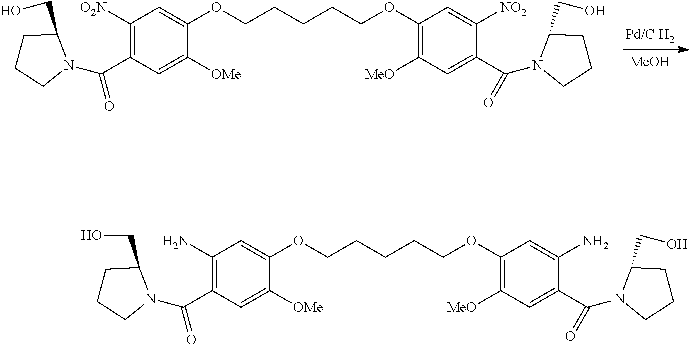 [SMILES: "MeOH" ; COc1cc(C(=O)N2CCC[C@H]2CO)c([N+](=O)[O-])cc1OCCCCCOc1cc([N+](=O)[O-])c(C(=O)N2CCC[C@H]2CO)cc1OC ; COc1cc(C(=O)N2CCC[C@H]2CO)c(N)cc1OCCCCCOc1cc(N)c(C(=O)N2CCC[C@H]2CO)cc1OC]

To a solution of (S)-((pentane-1,5-diylbis(oxy))bis(5-methoxy-2-nitro-4,1-phenylene))-bis(((S)-2-(hydroxymethyl)pyrrolidin-1-yl)methanone) (6.0 g, 9.1 mmol) in MeOH (100 mL) was added 10% Pd/C (2.4 g), the mixture was stirred under hydrogen atmosphere at room temperature overnight. After 14 h of stirring, the Pd/C was removed by filtration and washed with MeOH. The filtrate was concentrated and the residue was purified by chromatography (DCM/MeOH=10/1) to give (S)-((pentane-1,5-diylbis(oxy))bis(2-amino-5-methoxy-4,1-phenylene))bis(((S)-2-(hydroxymethyl)pyrrolidin-1-yl)methanone) (3.54 g, 5.9 mmol) as a white foam in 65% yield.

### Example 293. Synthesis of bis(4-((S)-2-((S)-2-(((allyloxy)carbonyl)amino)-3-methylbutanamido)propanamido)benzyl) ((S)-(pentane-1,5-diylbis(oxy))bis(2-((S)-2-(hydroxymethyl)pyrrolidine-1-carbonyl)-4-methoxy-5,1-phenylene))dicarbamate

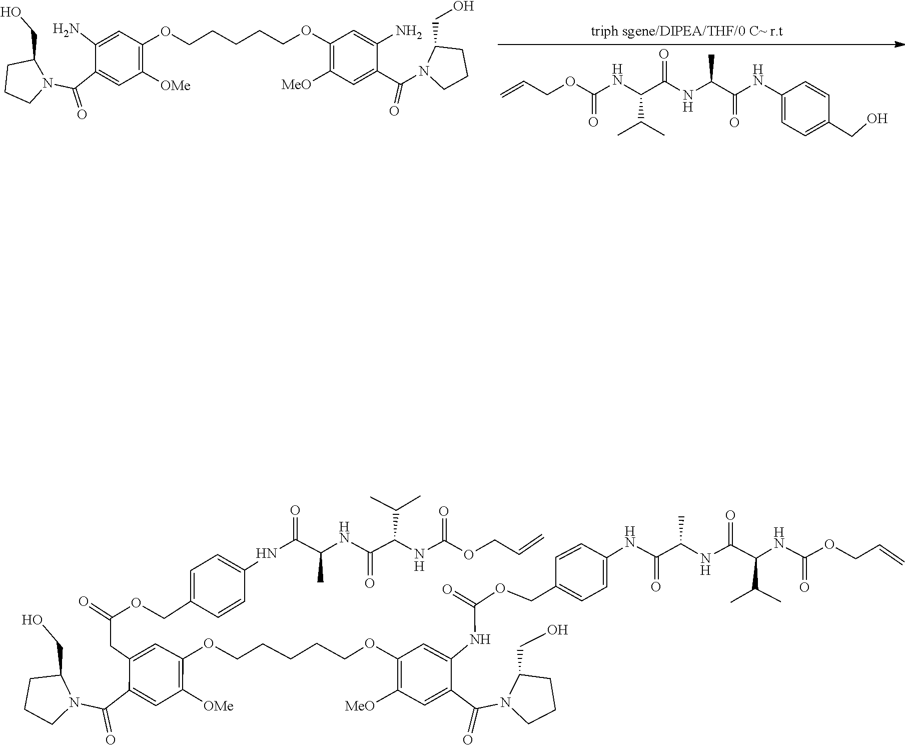

To a solution of allyl ((S)-1-(((S)-1-((4-(hydroxymethyl)phenyl)amino)-1-oxopropan-2-yl)amino)-3-methyl-1-oxobutan-2-yl)carbamate (8.0 g, 21.3 mmol) in dry THF (300 mL) was added DIPEA (5.5 g, 40.3 mmol) and a solution of triphosgene (3.2 g, 10.8 mmol) in dry THF (50 mL) at 5° C. After 15 min of stirring, the solution was recooled to 5° C. and a mixture of (S)-((pentane-1,5-diylbis(oxy))bis(2-amino-5-methoxy-4,1-phenylene))bis (((S)-2-(hydroxymethyl)-pyrrolidin-1-yl)methanone) (3.2 g, 5.3 mmol) and DIPEA (2.75 g, 21.6 mmol) in dry THE (150 mL) was added. The resultant solution was allowed to warm to room temperature and stirred overnight. The THF removed by evaporation in vacuo. The residue was purified by chromatography (DCM/MeOH=20/1) to give bis(4-((S)-2-((S)-2-(((allyloxy)carbonyl)amino)-3-methylbutanamido) propanamido)-benzyl)((S)-(pentane-1,5-diylbis(oxy))bis(2-((S)-2-(hydroxymethyl)pyrrolidine-1-carbonyl)-4-methoxy-5,1-phenylene))dicarbamate (7.0 g, 4.97 mmol) as a yellow foam in 94% yield.

### Example 294. Synthesis of (11S,11aS,11'S,11a'S)-bis(4-((S)-2-((S)-2-(((allyloxy)carbonyl)-amino)-3-methylbutanamido)propanamido)benzyl) 8,8′-(pentane-1,5-diylbis(oxy))bis(11-hydroxy-7-methoxy-5-oxo-2,3,11,11a-tetrahydro-1H-benzo[e]pyrrolo[1,2-a][1,4]diazepine-10(5H)-carboxylate)

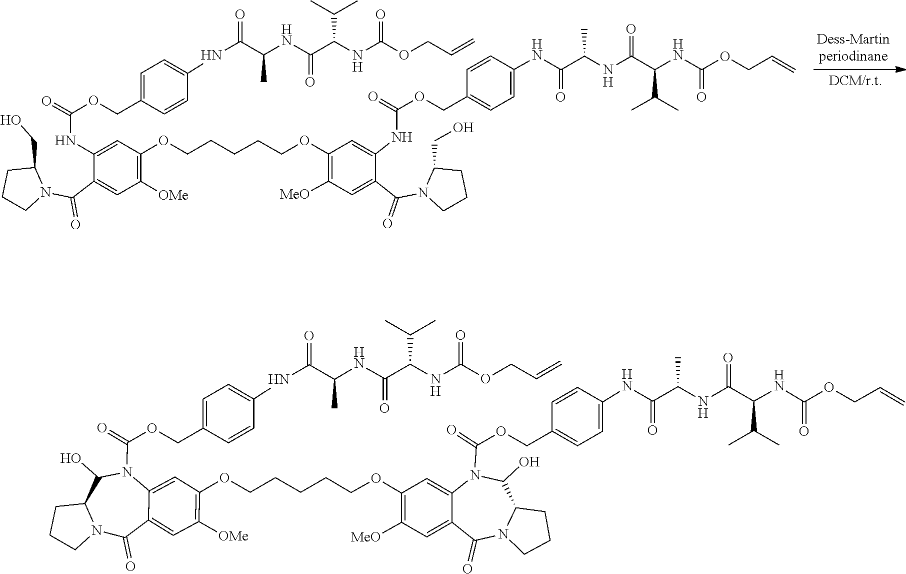

To a solution of bis(4-((S)-2-((S)-2-(((allyloxy)carbonyl)amino)-3-methyl butanamido) propanamido)benzyl)((S)-(pentane-1,5-diylbis(oxy))bis(2-((S)-2-(hydroxy-methyl)pyrrolidine-1-carbonyl)-4-methoxy-5,1-phenylene))dicarbamate (300 mg, 0.21 mmol) in dry DCM (15 mL) was added DMP (280 mg, 0.66 mmol) under nitrogen at room temperature. After completion of conversion, the reaction solution was added aqueous Na₂SO₃ and followed by aqueous NaHCO₃, the mixture was stirred for further 15 minutes and extracted with DCM (3×20 mL). The combined organic extract was washed with brine, dried, filtered and concentrated. The residue was purified by chromatography (DCM/MeOH=20/1) to give (11S,11aS,11'S,11′S)-bis(4-((S)-2-((S)-2-(((allyloxy)carbonyl)amino)-3-methylbutanamido)propanamido)benzyl)8,8′-(pentane-1,5-diylbis(oxy))bis(11-hydroxy-7-methoxy-5-oxo-2,3,11,11a-tetrahydro-1H-benzo[e]pyrrolo[1,2-a][1,4]diazepine-10(5H)-carboxylate) (270 mg, 0.19 mmol) as a off-white foam in 92% yield.

### Example 295. Synthesis of (11S,11aS,11'S,11a′S)-bis(4-((S)-2-((S)-2-(((allyloxy)carbonyl)-amino)-3-methylbutanamido)propanamido)benzyl) 8,8′-(pentane-1,5-diylbis(oxy))bis(11-hydroxy-7-methoxy-5-oxo-2,3,11,11a-tetrahydro-1H-benzo[e]pyrrolo[1,2-a][1,4]diazepine-10(5H)-carboxylate)

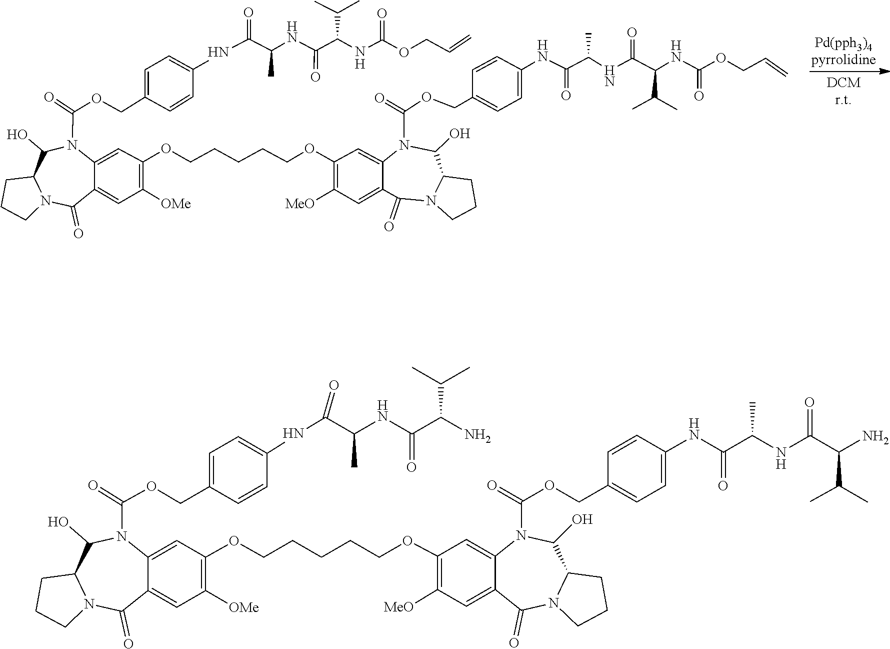

To a solution of (11S,11aS,11'S,11a'S)-bis(4-((S)-2-((S)-2-(((allyloxy)carbonyl) amino)-3-methylbutanamido)propanamido)benzyl)8,8′-(pentane-1,5-diylbis(oxy))bis(11-hydroxy-7-methoxy-5-oxo-2,3,11,11a-tetrahydro-1H-benzo[e]pyrrolo[1,2-a][1,4]diazepine-10(5H)-carboxylate) (774 mg, 0.55 mmol) and pyrrolidine (196 mg, 2.76 mmol) in dry DCM (8 mL) was added Pd(pph₃)₄ (76 mg, 0.066 mmol). The reaction was flushed with argon and stirred for 2h at room temperature, after which the reaction was diluted with DCM and washed sequentially with saturated aqueous NH₄Cl and brine. The organic phase was dried over Na₂SO₄, filtered and concentrated. The residue was purified by chromatography (DCM/MeOH=6/1) to give (11S,11aS,11'S,11a'S)-bis(4-((S)-2-((S)-2-(((allyloxy) carbonyl)amino)-3-methyl-butanamido)propanamido)benzyl)8,8′-(pentane-1,5-diylbis(oxy))bis(11-hydroxy-7-methoxy-5-oxo-2,3,11,11a-tetrahydro-1H-benzo[e]-pyrrolo[1,2-a][1,4]diazepine-10(5H)-carboxylate) (420 mg, 0.34 mmol) as an off-white solid in 62% yield.

### Example 296. Synthesis of (S)-2-(((allyloxy)carbonyl)amino)-3-methylbutanoic Acid

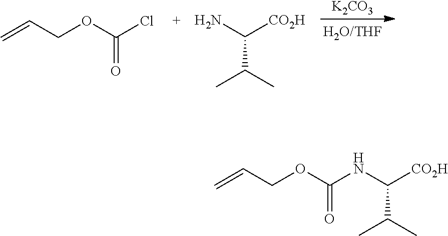

Allyl chloroformate (24.8 g, 205 mmol) was added dropwise to a stirred solution of L-valine (20 g, 171 mmol) and K₂CO₃ (35.4 g, 257 mmol) in H₂O (250 mL) and THF (250 mL). The reaction mixture was stirred at room temperature overnight, then the solvent was concentrated under reduced pressure and the remaining solution extracted with diethyl ether (100 mL). The aqueous portion was acidified to pH 2 with conc. HCl and extracted with DCM (3×200 mL). The combined organics were washed with brine, dried over Na₂SO₄, filtered and concentrated to afford the product (35 g, 174 mmol). White solid, yield (100%).

### Example 297. Synthesis of (S)-2,5-dioxopyrrolidin-1-yl 2-(((allyloxy)carbonyl)amino)-3-methylbutanoate

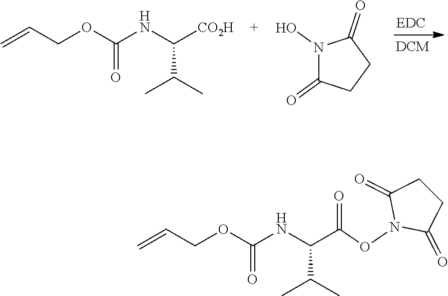 [SMILES: C=CCOC(=O)N[C@H](C(=O)O)C(C)C ; O=C1CCC(=O)N1O ; C=CCOC(=O)N[C@H](C(=O)ON1C(=O)CCC1=O)C(C)C]

To a stirred solution of (S)-2-(((allyloxy)carbonyl)amino)-3-methylbutanoic acid (35 g, 174 mmol) in dry DCM (500 mL) was added EDC (66.9 g, 348 mmol) and N-hydroxysuccinimide (30 g, 261 mmol) at room temperature. After 14 h of stirring, the reaction was diluted with DCM and washed with water and brine. The organic phase was dried over Na₂SO₄, filtered and concentrated to afforded the product (54.5 g) which was used in the next step without further purification. Yield: (100%) viscous colourless oil. R_{f}=0.5 (PE/EA=2/1)

### Example 298. Synthesis of (S)-2-((S)-2-(((allyloxy)carbonyl)amino)-3-methylbutanamido)-propanoic Acid

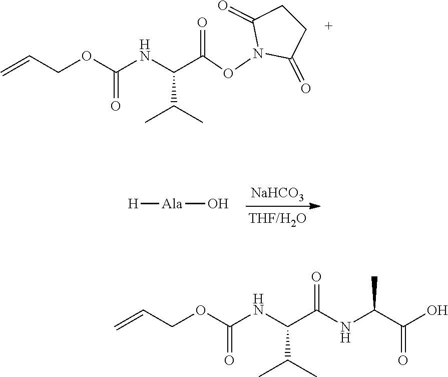

To a solution of H-Ala-OH (15.7 g, 176 mmol) and NaHCO₃ (15.5 g, 185 mmol) in THE (200 mL) and H₂O (200 mL) was added a solution of (S)-2,5-dioxopyrrolidin-1-yl 2-(((allyloxy)-carbonyl)amino)-3-methylbutanoate (50 g, 168 mmol) in THE (100 mL) at room temperature. After 72 hours of stirring, the THF was evaporated under reduced pressure. The residue was acidified to pH 3 with citric acid and extracted with EA (3×350 mL), the combined extracts was washed with brine, dried, filtered and concentrated to give a white solid. Trituration with diethyl ether (excess) afforded the pure product as a white powder (25.2 g, 93 mmol, 55%).

### Example 299. Synthesis of allyl ((S)-1-(((S)-1-((4-(hydroxymethyl)phenyl)amino)-1-oxopropan-2-yl)amino)-3-methyl-1-oxobutan-2-yl)carbamate

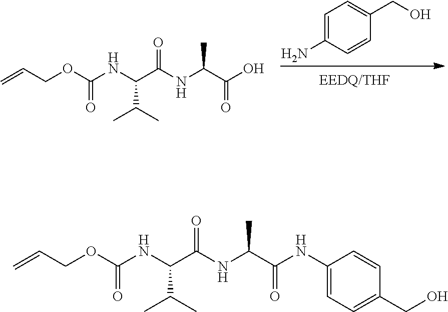 [SMILES: C=CCOC(=O)N[C@H](C(=O)N[C@@H](C)C(=O)O)C(C)C ; Nc1ccc(CO)cc1 ; C=CCOC(=O)N[C@H](C(=O)N[C@@H](C)C(=O)Nc1ccc(CO)cc1)C(C)C]

To a solution of (S)-2-((S)-2-(((allyloxy)carbonyl)amino)-3-methylbutanamido)-propanoic acid (25.2 g, 92.6 mmol) and p-aminobenzyl alcohol (12.0 g, 97.6 mmol) in THF (300 mL) was added EEDQ (24.0 g, 97.2 mmol) at room temperature. After 18 hours of stirring, the solvent was evaporated under reduced pressure to give a pale brown solid. The solid was triturated with diethyl ether and filtered, washing with an excess of diethyl ether. This afforded the product as a white solid (40 g, 106 mmol, 100%).

### Example 300. Synthesis of 4-(((benzyloxy)carbonyl)amino)butanoic Acid

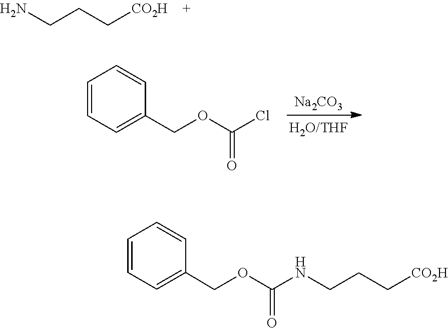

Na₂CO₃ (41.1 g, 387 mmol) was added to a solution of 4-aminobutanoic acid (20 g, 193 mmol) in H₂O (300 mL) at 5° C. After 10 min of stirring, a solution of CbzCl (33.2 mL, 232 mmol) in THF (100 mL) was added dropwise. The reaction was allowed to warm to room temperature and stirred overnight. After completion of conversion, the mixture was diluted with H₂O (100 mL) and extracted with EA (2×100 mL). The aqueous layer was acidified to pH 2 with conc. HCl and extracted with EA (3×100 mL). The combined organics were washed with brine, dried over Na₂SO₄, filtered and concentrated to give a white solid. Trituration with PE (excess) afforded the pure product as a white powder (31.6 g, 70%).

### Example 301. Synthesis of tert-butyl 4-(((benzyloxy)carbonyl)amino)butanoate

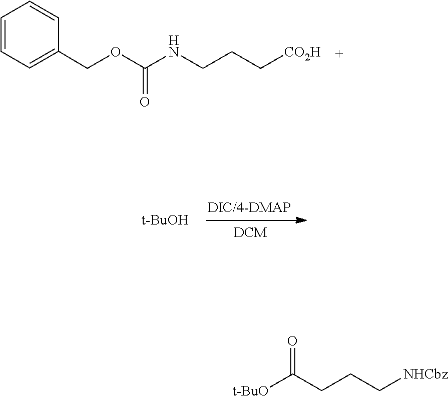 [SMILES: "t-BuOH" ; O=C(O)CCCNC(=O)OCc1ccccc1 ; CC(C)(C)OC(=O)CCCNC(=O)OCc1ccccc1]

To a stirred solution of 4-(((benzyloxy)carbonyl)amino)butanoic acid (5.9 g, 24.9 mmol) and tert-Butanol (14.7 g, 199 mmol) in dry DCM (250 mL) was added 4-DMAP (0.61 g, 5 mmol) and DIC (4.7 g, 37.3 mmol) at 0° C. After 16 h of stirring, the reaction was filtered and extracted with DCM (2×200 mL). The combined organic extract was washed with 1N HCl and brine, dried over Na₂SO₄, filtered and concentrated. The residue was purified by chromatography (100% DCM) to give tert-butyl 4-(((benzyloxy)carbonyl)amino)butanoate (4.26 g, 14.5 mmol, 58%) viscous colourless oil.

### Example 302. Synthesis of tert-butyl 4-aminobutanoate

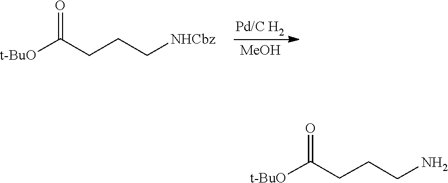 [SMILES: "MeOH" ; CC(C)(C)OC(=O)CCCNC(=O)OCc1ccccc1 ; CC(C)(C)OC(=O)CCCN]

To a solution of tert-butyl 4-(((benzyloxy)carbonyl)amino)butanoate (1.69 g, 5.77 mmol) in MeOH (40 mL) was added 10% Pd/C (400 mg), the mixture was stirred under hydrogen atmosphere at room temperature overnight. After 14 h of stirring, the Pd/C was removed by filtration and washed with MeOH. The filtrate was concentrated to afford the product which was used in the next step without further purification (897 mg, 5.64 mmol). colorless liquid, yield (98%).

### Example 303. Synthesis of (2R,3S)-2,3-bis(benzylamino)succinic Acid

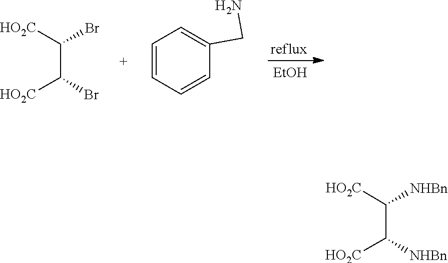

To a solution of meso-2,3-dibromosuccinic acid (50 g, 181 mmol) in EtOH (400 mL) was added benzylamine (150 mL) dropwise. After completion of addition, the mixture was heated to 90° C. and stirred overnight. The mixture was cooled to room temperature and diluted with H₂O. 6N HCl was added until pH 4 to give white precipitates. The precipitates were filtered, rinsed with H₂O and dried to give (2R,3S)-2,3-bis(benzylamino)succinic acid (50 g, 152 mmol, 84%).

### Example 304. Synthesis of (2R,3S)-2,3-diaminosuccinic Acid

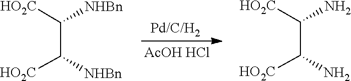

To a solution of (2R,3S)-2,3-bis(benzylamino)succinic acid (18 g, 55 mmol) in AcOH (100 mL) and HCl (100 mL) was added 10% Pd/C (3 g), the mixture was stirred under hydrogen atmosphere at 50° C. overnight. After 48 h of stirring, the Pd/C was removed by filtration and washed with H₂O. The filtrate was concentrated and the residue was dissolved in 1N NaOH (200 mL). AcOH was added until pH 5 to give white precipitates. The precipitates were filtered, rinsed with H₂O and dried to give (2R,3S)-2,3-diaminosuccinic acid (8.7 g, 58.8 g, 100%).

### Example 305. Synthesis of 2,3-bis(((benzyloxy)carbonyl)amino)succinic Acid

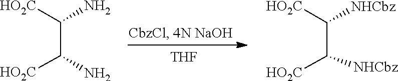 [SMILES: N[C@H](C(=O)O)[C@@H](N)C(=O)O ; O=C(N[C@H](C(=O)O)[C@@H](NC(=O)OCc1ccccc1)C(=O)O)OCc1ccccc1 ; "THF"]

To a solution of (2R,3S)-2,3-diaminosuccinic acid (31.74 g, 214 mmol) in THE (220 mL) and 4N NaOH (214 mL) was added CbzCl (61 mL, 428 mmol) dropwise at 0° C. After completion of addition, the mixture was allowed to warm to room temperature and stirred for 2 h. The reaction was diluted with H₂O (1600 mL) and extracted with EA (2×15600 mL). The aqueous layer was separated and acidified with conc.HCl until pH 2 was reached. The resultant solution was stirred for 1 h and standed at 5° C. to give white precipitates. The precipitates were filtered, rinsed with H₂O and dried to give 2,3-bis(((benzyloxy)carbonyl)amino)succinic acid (52.2 g, 125 mmol, 59%).

### Example 306. Synthesis of dibenzyl ((3R,4S)-2,5-dioxotetrahydrofuran-3,4-diyl)dicarbamate

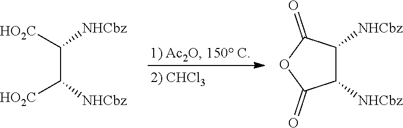 [SMILES: O=C(N[C@H](C(=O)O)[C@@H](NC(=O)OCc1ccccc1)C(=O)O)OCc1ccccc1 ; O=C(N[C@@H]1C(=O)OC(=O)[C@@H]1NC(=O)OCc1ccccc1)OCc1ccccc1]

The solution of 2,3-bis(((benzyloxy)carbonyl)amino)succinic acid (5.0 g, 12 mmol) in Ac₂O (37.5 mL) was refluxed for 20 min, cooled and concentrated to give resulting anhydride. The diastereomeric mixture was treat with CHCl₃ (37 mL), the insoluble meso-isomer was filtered and washed with PE to give crystals of dibenzyl ((3R,4S)-2,5-dioxotetrahydrofuran-3,4-diyl)dicarbamate (2.0 g, 5 mmol, 42%)

### Example 307. Synthesis of di-tert-butyl 4,4′-(((2R,3S)-2,3-bis(((benzyloxy)carbonyl)-amino)succinyl)bis(azanediyl))dibutanoate

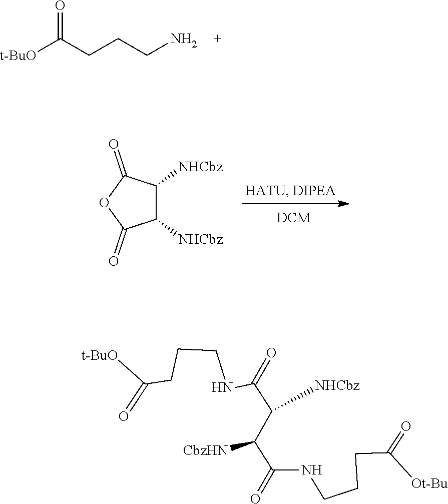 [SMILES: CC(C)(C)OC(=O)CCCN ; O=C(N[C@@H]1C(=O)OC(=O)[C@@H]1NC(=O)OCc1ccccc1)OCc1ccccc1 ; CC(C)(C)OC(=O)CCCNC(=O)[C@@H](NC(=O)OCc1ccccc1)[C@@H](NC(=O)OCc1ccccc1)C(=O)NCCCC(=O)OC(C)(C)C]

To a solution of dibenzyl ((3R,4S)-2,5-dioxotetrahydrofuran-3,4-diyl)dicarbamate (2.03 g, 5.1 mmol) and tert-butyl 4-aminobutanoate (1.79 g, 11.3 mmol) in DMF (45 mL) was added DIPEA (1.98 g, 15.3 mmol) at 0° C. After 5 min of stirring, HATU (4.66 g, 12.3 mmol) was added. The mixture was allowed to warm to room temperature and stirred for 2 h. After completion of conversion, the mixture was diluted with H₂O (90 mL) and extracted with EA (2×200 mL) and DCM (2×90 mL), the combined organic extract was washed with brine and dried over Na₂SO₄. The majority of solvent was removed under reduced pressure and a white solid was precipitated, which was collected and dried to give di-tert-butyl 4,4′-(((2R,3S)-2,3-bis (((benzyloxy)carbonyl)amino)succinyl)bis(azanediyl))dibutanoate (2.8 g, 4.0 mmol) as a white solid in 80% yield.

### Example 308. Synthesis of di-tert-butyl 4,4′-(((2R,3S)-2,3-diaminosuccinyl)bis-(azanediyl))dibutanoate

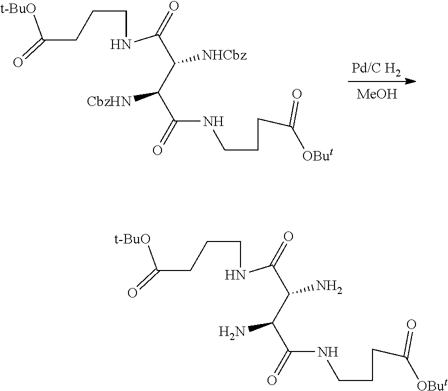

To a solution of 4,4′-(((2R,3S)-2,3-bis(((benzyloxy)carbonyl)amino)succinyl)bis-(azanediyl))dibutanoate (2.8 g, 4.0 mmol) in MeOH (100 mL) was added 10% Pd/C (1.1 g), the mixture was stirred under hydrogen atmosphere at room temperature overnight. After 18 h of stirring, the Pd/C was removed by filtration and washed with MeOH. The filtrate was concentrated to afford the product which was used in the next step without further purification (940 mg, 2.2 mmol). colorless liquid, yield (55%).

### Example 309. Synthesis of di-tert-butyl 4,4′-(((2R,3S)-2,3-bis(4-(2,5-dioxo-2,5-dihydro-1H-pyrrol-1-yl)butanamido)succinyl)bis(azanediyl))dibutanoate

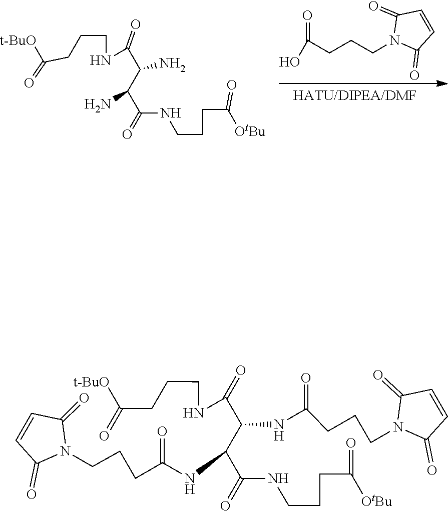 [SMILES: CC(C)(C)OC(=O)CCCNC(=O)[C@@H](N)[C@@H](N)C(=O)NCCCC(=O)OC(C)(C)C ; O=C(O)CCCN1C(=O)C=CC1=O ; CC(C)(C)OC(=O)CCCNC(=O)[C@@H](NC(=O)CCCN1C(=O)C=CC1=O)[C@@H](NC(=O)CCCN1C(=O)C=CC1=O)C(=O)NCCCC(=O)OC(C)(C)C]

To a solution of di-tert-butyl 4,4′-(((2R,3S)-2,3-diaminosuccinyl)bis(azanediyl))-dibutanoate (940 mg, 2.19 mmol) and 4-(2,5-dioxo-2,5-dihydro-1H-pyrrol-1-yl)butanoic acid (840 mg, 4.59 mmol) in DMF (25 mL) was added DIPEA (1.13 g, 8.76 mmol) at 0° C. After 5 min of stirring, HATU (1.74 g, 4.58 mmol) was added. The mixture was allowed to warm to room temperature and stirred for 1 h. After completion of conversion, the mixture was diluted with H₂O (50 mL) and extracted with EA (2×100 mL) and DCM (2×50 mL), the combined organic extracts were washed with brine and dried over Na₂SO₄. The majority of solvent was removed under reduced pressure and a white solid was precipitated, which was collected and dried to give di-tert-butyl 4,4′-(((2R,3 S)-2,3-bis(4-(2,5-dioxo-2,5-dihydro-1H-pyrrol-1-yl)butanamido)succinyl)bis-(azanediyl))dibutanoate (1.36 g, 1.79 mmol) as a white solid in 82% yield.

### Example 310. Synthesis of 4,4′-(((2R,3S)-2,3-bis(4-(2,5-dioxo-2,5-dihydro-1H-pyrrol-1-yl)butanamido)succinyl) bis(azanediyl))dibutanoic Acid

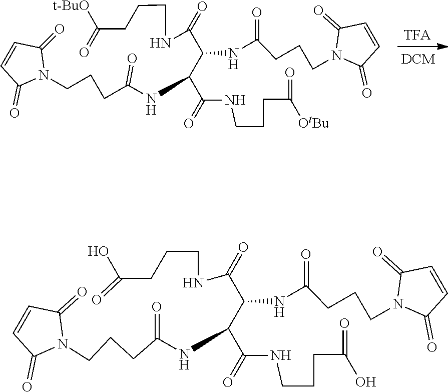 [SMILES: CC(C)(C)OC(=O)CCCNC(=O)[C@@H](NC(=O)CCCN1C(=O)C=CC1=O)[C@@H](NC(=O)CCCN1C(=O)C=CC1=O)C(=O)NCCCC(=O)OC(C)(C)C ; O=C(O)CCCNC(=O)[C@@H](NC(=O)CCCN1C(=O)C=CC1=O)[C@@H](NC(=O)CCCN1C(=O)C=CC1=O)C(=O)NCCCC(=O)O]

To a solution of di-tert-butyl 4,4′-(((2R,3S)-2,3-bis(4-(2,5-dioxo-2,5-dihydro-1H-pyrrol-1-yl)butanamido)succinyl)bis(azanediyl))dibutanoate (1.36 g, 1.79 mmol) in DCM (15 mL) was added TFA (30 mL) at room temperature 0° C. After 18 h of stirring, the reaction was concentrated and the residue was dissolved in dry toluene. The solvent was removed by evaporation in vacuo to give white precipitates which was used in the next step without further purification (1.3 mg, 2.0 mmol). yield (100%).

### Example 311. Synthesis of PBD Product C-07

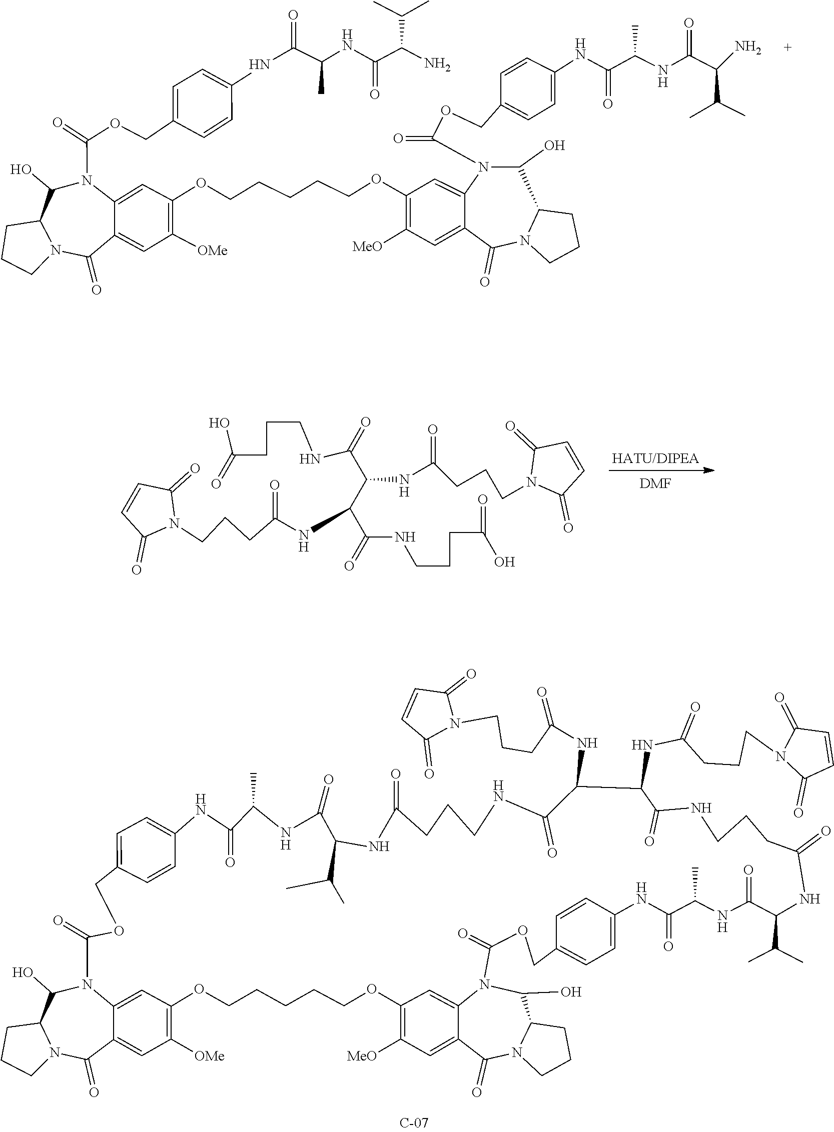

To a solution of (11S,11aS,11′S,11a′S)-bis(4-((S)-2-((S)-2-(((allyloxy)carbonyl) amino)-3-methylbutanamido)propanamido)benzyl)8,8′-(pentane-1,5-diylbis(oxy))bis(11-hydroxy-7-methoxy-5-oxo-2,3,11,11a-tetrahydro-1H-benzo[e]pyrrolo[1,2-a][1,4]diazepine-10(5H)-carboxylate) (215 mg, 0.17 mmol) and 4,4′-(((2R,3S)-2,3-bis(4-(2,5-dioxo-2,5-dihydro-1H-pyrrol-1-yl)butanamido)succinyl)bis(azanediyl))dibutanoic acid (115 mg, 0.18 mmol) in DMF (18 mL) was added DIPEA (90 mg, 0.70 mmol) at 0 TC. After 5 min of stirring, HATU (132 mg, 0.35 mmol) was added. The mixture was allowed to warm to room temperature and stirred overnight. After completion of conversion, the mixture was diluted with H₂ (2 mL) and extracted with EA (2×40 mL) and DCM (2×20 mL), the combined organic extract was washed with brine, dried, filtered and concentrated. The residue was purified by pre-HPLC to give PBD product C-07 (10 mg) as a white powder. ESI MS m/z C₉₁H₁₁₅N₁₆O₂₆ [M+H]⁺, cacld. 1847.81, found 1847.60.

### Example 312. Synthesis of di-tert-butyl 4,4′-(((2R,3S)-2,3-bis(4-(2,5-dioxo-2,5-dihydro-1H-pyrrol-1-yl)butanamido)succinyl)bis(azanediyl))dibutanoate

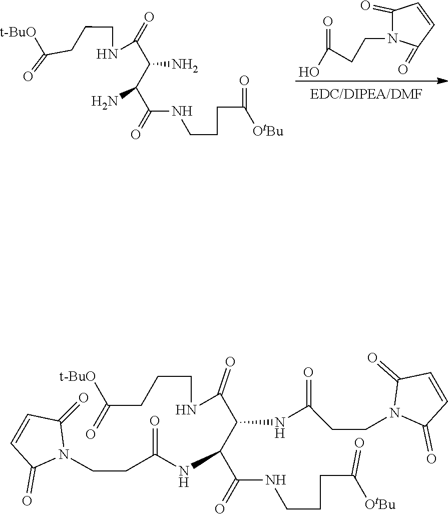 [SMILES: CC(C)(C)OC(=O)CCCNC(=O)[C@@H](N)[C@@H](N)C(=O)NCCCC(=O)OC(C)(C)C ; O=C(O)CCN1C(=O)C=CC1=O ; CC(C)(C)OC(=O)CCCNC(=O)[C@@H](NC(=O)CCN1C(=O)C=CC1=O)[C@@H](NC(=O)CCN1C(=O)C=CC1=O)C(=O)NCCCC(=O)OC(C)(C)C]

To a solution of di-tert-butyl 4,4′-(((2R,3S)-2,3-diaminosuccinyl)bis(azanediyl))-dibutanoate (900 mg, 2.09 mmol) and 3-(2,5-dioxo-2,5-dihydro-1H-pyrrol-1-yl)propanoic acid (840 mg, 4.97 mmol) in DMF (25 mL) was added DIPEA (0.93 g, 7.21 mmol) at 0° C. After 5 min of stirring, EDC (1.74 g, 9.06 mmol) was added. The mixture was allowed to warm to room temperature and stirred for 1 h. After completion of conversion, the mixture was diluted with H₂O (50 mL) and extracted with EA (2×100 mL) and DCM (2×50 mL), the combined organic extracts were washed with brine and dried over Na₂SO₄. The majority of solvent was removed under reduced pressure and a white solid was precipitated, which was collected and dried to give di-tert-butyl 4,4′-(((2R,3 S)-2,3-bis(3-(2,5-dioxo-2,5-dihydro-1H-pyrrol-1-yl)propanamido)succinyl)bis-(azanediyl))dibutanoate (1.27 g, 1.79 mmol) as a white solid in 83% yield. ESI MS m/z+C₃₄H₄₉N₆O₁₂, cacld. 733.33 (M+H), found 733.55.

### Example 313. Synthesis of4,4′-(((2R,3S)-2,3-bis(3-(2,5-dioxo-2,5-dihydro-1H-pyrrol-1-yl)propanamido)succinyl)bis(azanediyl))dibutanoic Acid

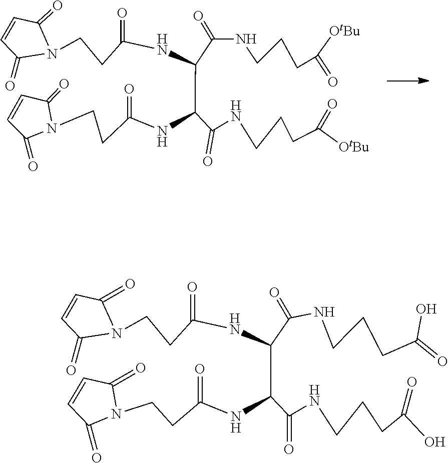 [SMILES: CC(C)(C)OC(=O)CCCNC(=O)[C@@H](NC(=O)CCN1C(=O)C=CC1=O)[C@@H](NC(=O)CCN1C(=O)C=CC1=O)C(=O)NCCCC(=O)OC(C)(C)C ; O=C(O)CCCNC(=O)[C@@H](NC(=O)CCN1C(=O)C=CC1=O)[C@@H](NC(=O)CCN1C(=O)C=CC1=O)C(=O)NCCCC(=O)O]

Di-tert-butyl 4,4′-(((2R,3S)-2,3-bis(4-(2,5-dioxo-2,5-dihydro-1H-pyrrol-1-yl)butanamido)succinyl)bis(azanediyl))dibutanoate (502.0 mg, 0.685 mmol) in 1,4-dioxane (8 ml) at 4° C. was added conc. HCl (3 ml). The mixture was then stirred at RT for 30 min, diluted with 1,4-dioxane (8 ml), concentrated, co-evaporated with dioxane/toluene (1:1, 2×10 ml) to dryness and crystallized with EtOH/Hexane to afford the title compound (289.0 g, 68% yield). ESI MS m/z+C₂₆H₃₃N₆O₁₂, cacld. 621.21 (M+H), found 621.55.

### Example 314. Synthesis of allyl ((S)-3-methyl-1-(((S)-1-((4-((((4-nitrophenoxy)carbonyl)-oxy)methyl)phenyl)amino)-1-oxopropan-2-yl)amino)-1-oxobutan-2-yl)carbamate

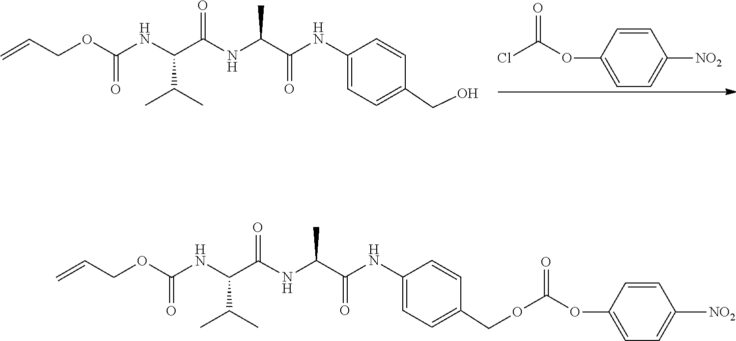 [SMILES: C=CCOC(=O)N[C@H](C(=O)N[C@@H](C)C(=O)Nc1ccc(CO)cc1)C(C)C ; O=C(Cl)Oc1ccc([N+](=O)[O-])cc1 ; C=CCOC(=O)N[C@H](C(=O)N[C@@H](C)C(=O)Nc1ccc(COC(=O)Oc2ccc([N+](=O)[O-])cc2)cc1)C(C)C]

Aly ((S)-1-(((S)-1-((4-(hydroxymethyl)phenyl)amino)-1-oxopropan-2-yl)amino)-3-methyl-1-oxobutan-2-yl)carbamate (2.21 g, 5.86 mmol) in the mixture of dry pyridine (5 ml) and CH₂Cl₂ (20 ml) was added 4-nitrophenyl carbonochloridate (1.82 g, 9.05 mmol). The mixture was stirred at RT for 8 hour, concentrated and purified on SiO₂ column eluted with EtOAc/CH₂Cl₂ (1:12) to afford the title compound (2.63 g, 83% yield). MS ESI m/z calcd for C₂₆H₃₁N₄O₉ [M+H]⁺ 543.21, found 543.60.

### Example 315. Synthesis of (11aS,11a'S)-bis(4-((S)-2-((S)-2-(((allyloxy)carbonyl)amino)-3-methylbutanamido)propanamido)benzyl) 8,8′-(pentane-1,5-diylbis(oxy))bis(7-methoxy-2-methylene-5-oxo-2,3,11,11a-tetrahydro-1H-benzo[e]pyrrolo[1,2-a][1,4]diazepine-10(5H)-carboxylate)

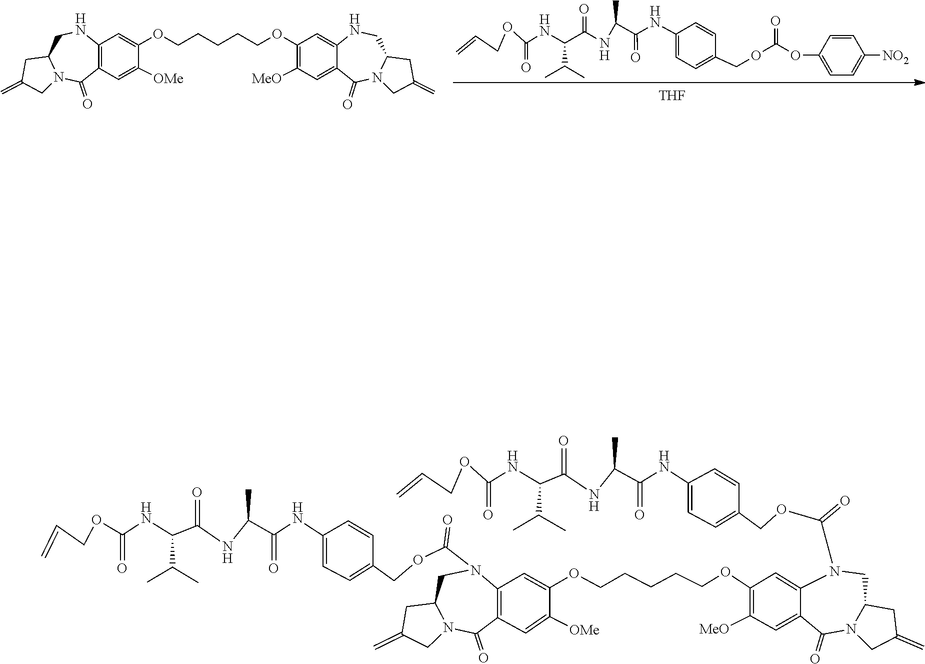

(11aS,11a'S)-8,8′-(Pentane-1,5-diylbis(oxy))bis(7-methoxy-2-methylene-2,3,11,11a-tetrahydro-1H-benzo[e]pyrrolo[1,2-a][1,4]diazepin-5(10H)-one) (288.2 mg, 0.490 mmol) in dry CH₃CN (5 ml) was added allyl ((S)-3-methyl-1-(((S)-1-((4-((((4-nitrophenoxy)carbonyl)oxy)-methyl)phenyl)amino)-1-oxopropan-2-yl)amino)-1-oxobutan-2-yl)carbamate (770.2 mg, 1.420 mmol) and DIPEA (2 ml). The mixture was stirred at 45° C. for 8 h, concentrated and purified on SiO₂ column eluted with EtOAc/CH₂Cl₂ (1:8) to afford the title compound (492.0 mg, 72% yield). MS ESI m/z calcd for C₇₃H₉₁N₁₀O₁₈ [M+H]⁺ 1395.64, found 1395.95.

### Example 316. Synthesis of (11aS,11a'S)-bis(4-((S)-2-((S)-2-amino-3-methylbutanamido)-propanamido)benzyl) 8,8′-(pentane-1,5-diylbis(oxy))bis(7-methoxy-2-methylene-5-oxo-2,3,11,11a-tetrahydro-1H-benzo[e]pyrrolo[1,2-a][1,4]diazepine-10(5H)-carboxylate)

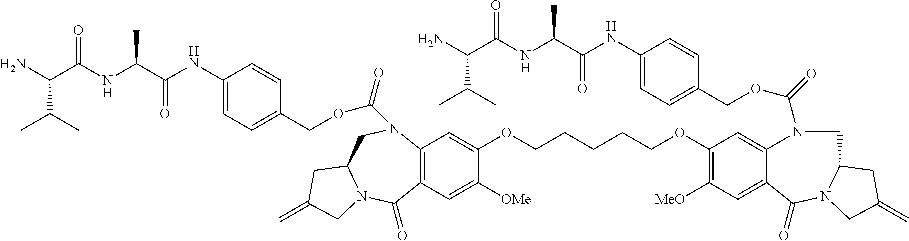 [SMILES: C=C1C[C@H]2CN(C(=O)OCc3ccc(NC(=O)[C@H](C)NC(=O)[C@@H](N)C(C)C)cc3)c3cc(OCCCCCOc4cc5c(cc4OC)C(=O)N4CC(=C)C[C@H]4CN5C(=O)OCc4ccc(NC(=O)[C@H](C)NC(=O)[C@@H](N)C(C)C)cc4)c(OC)cc3C(=O)N2C1]

To a solution of (11aS,11a'S)-bis(4-((S)-2-((S)-2-(((allyloxy)carbonyl)amino)-3-methylbutanamido)propanamido)benzyl) 8,8′-(pentane-1,5-diylbis(oxy))bis(7-methoxy-2-methylene-5-oxo-2,3,11,11a-tetrahydro-1H-benzo[e]pyrrolo[1,2-a][1,4]diazepine-10(5H)-carboxylate) (274.2 mg, 0.197 mmol) and pyrrolidine (49 mg, 6.90 mmol) in dry DCM (5 mL) was added Pd(pph₃)₄ (152.0 mg, 0.132 mmol). The reaction was flushed with argon and stirred for 2h at room temperature, after which the reaction was diluted with DCM and washed sequentially with saturated aqueous NH₄Cl and brine. The organic phase was dried over Na₂SO₄, filtered and concentrated. The residue was purified by chromatography (DCM/MeOH/Et₃N=6/1/0.02) to give the title compound (166.7 mg, 69% yield) as an off-white solid. MS ESI m/z calcd for C₆₅H₈₃N₁₀O₁₄ [M+H]⁺ 1227.60, found 1227.93.

### Example 317. Synthesis of PBD Product C-08

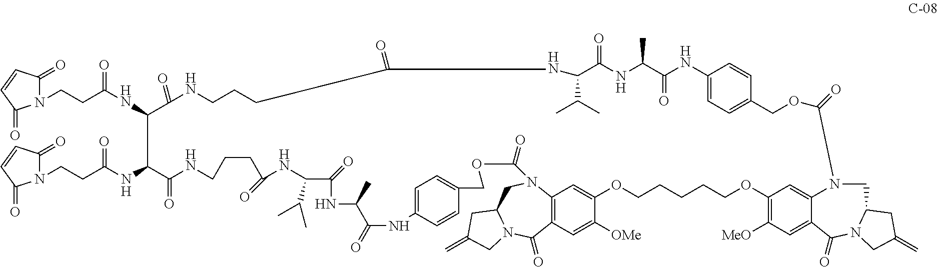 [SMILES: C=C1C[C@H]2CN3C(=O)OCc4ccc(cc4)NC(=O)[C@H](C)NC(=O)[C@H](C(C)C)NC(=O)CCCNC(=O)[C@@H](NC(=O)CCN4C(=O)C=CC4=O)[C@@H](NC(=O)CCN4C(=O)C=CC4=O)C(=O)NCCCC(=O)N[C@@H](C(C)C)C(=O)N[C@@H](C)C(=O)Nc4ccc(cc4)COC(=O)N4C[C@@H]5CC(=C)CN5C(=O)c5cc(OC)c(cc54)OCCCCCOc4cc3c(cc4OC)C(=O)N2C1]

(11aS,11a′S)-Bis(4-((S)-2-((S)-2-amino-3-methylbutanamido)-propanamido)benzyl) 8,8′-(pentane-1,5-diylbis(oxy))bis(7-methoxy-2-methylene-5-oxo-2,3,11,11a-tetrahydro-1H-benzo[e]pyrrolo[1,2-a][1,4]diazepine-10(5H)-carboxylate) (151.1 mg, 0.123 mmol) and 4,4′-(((2R,3S)-2,3-bis(3-(2,5-dioxo-2,5-dihydro-1H-pyrrol-1-yl)propanamido)succinyl)bis-(azanediyl))dibutanoic acid (77.1 mg, 0.124 mmol) in DMA (5 ml) was added EDC (95.2 mg, 0.496 mmol). The mixture was stirred at RT for 8 h, concentrated and purified on C-18 HPLC C18 3 μm column (25×4 cm) using gradient elution with a mixture of (A) acetonitrile and (B) water/0.1% formic acid (gradient: 15% A: 85% B up to 25% A: 75% B over 5 minutes, 35% A:65% B for 15 minutes, 60% A: 40% B down to 50% A: 50% B over 15 minute, 15% A: 85% B for 5 minutes) with a 8 mL/minute flow rate. The fractions containing the title compound were pooled, evaporated and dried in a desiccator with P₂O₅ to afford the C-8 PBD compound (149.2 mg, 67% yield). MS ESI m/z calcd for C₉₁H₁₁₁N₁₆O₂₄ [M+H]⁺ 1811.79, found 1812.35.

### Example 318. Synthesis of (S)-(4-(benzyloxy)-5-methoxy-2-nitrophenyl)(2-(hydroxyl-ethyl)pyrrolidin-1-yl)methanone

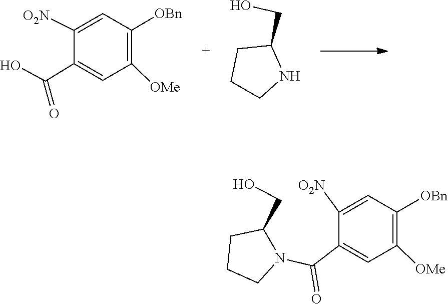 [SMILES: COc1cc(C(=O)O)c([N+](=O)[O-])cc1OCc1ccccc1 ; OC[C@@H]1CCCN1 ; COc1cc(C(=O)N2CCC[C@H]2CO)c([N+](=O)[O-])cc1OCc1ccccc1]

4-(benzyloxy)-5-methoxy-2-nitrobenzoic acid (10.20 g, 33.65 mmol) and (S)-pyrrolidin-2-ylmethanol (3.85 g, 38.09 mmol) in dry DMF (150 ml) was added EDC (19.50 g, 101.56 mmol). The mixture was stirred at RT for overnight, concentrated and purified on SiO₂ column eluted with EtOAc/CH₂Cl₂ (1:4) to afford the title compound (11.56 g, 89% yield). MS ESI m/z calcd for C₂₀H₂₃N₂O₆ [M+H]⁺ 387.15, found 387.65.

### Example 319. Synthesis of (S)-1-(4-(benzyloxy)-5-methoxy-2-nitrobenzoyl)pyrrolidine-2-carbaldehyde

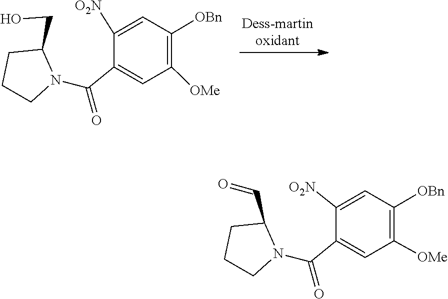 [SMILES: COc1cc(C(=O)N2CCC[C@H]2CO)c([N+](=O)[O-])cc1OCc1ccccc1 ; COc1cc(C(=O)N2CCC[C@H]2C=O)c([N+](=O)[O-])cc1OCc1ccccc1]

To a solution of (S)-(4-(benzyloxy)-5-methoxy-2-nitrophenyl)(2-(hydroxymethyl)-pyrrolidin-1-yl)methanone (3.80 g, 9.84 mmol) in dry DCM (15 mL) was added Dess-Martin periodinane (DMP) (5.80 g, 13.67 mmol) under nitrogen at room temperature. After completion of conversion, the reaction solution was added aqueous Na₂SO₃ and followed by aqueous NaHCO₃, the mixture was stirred for further 15 minutes and extracted with DCM (3×20 mL). The combined organic extract was washed with brine, dried, filtered and concentrated. The residue was purified by SiO₂ chromatography (DCM/EtOAc=4/1) to give the title compound (3.13 g, 83% yield) as an off-white foam. MS ESI m/z calcd for C₂₀H₂₁N₂O₆ [M+H]⁺ 385.13, found 385.60, 404.75 [M+H₂O+H]⁺.

### Example 320. Synthesis of 8-hydroxy-7-methoxy-2,3,11,11a-tetrahydro-1H-benzo[e]pyrrolo[1,2-a][1,4]diazepin-5(10H)-one

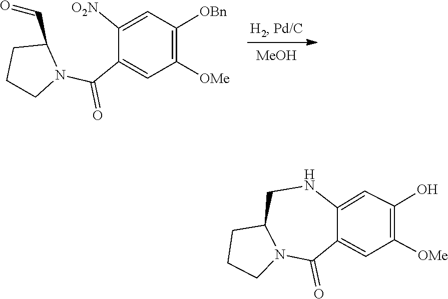 [SMILES: "MeOH" ; COc1cc(C(=O)N2CCC[C@H]2C=O)c([N+](=O)[O-])cc1OCc1ccccc1 ; COc1cc2c(cc1O)NC[C@@H]1CCCN1C2=O]

To a solution of S)-1-(4-(benzyloxy)-5-methoxy-2-nitrobenzoyl)pyrrolidine-2-carbaldehyde (3.00 g, 7.80 mmol) in methanol (75 mL) was Pd/C (10% Pd, 50% wet, 250 mg) in a hydrogenation shaker. After air in the shaker was vacuumed out, hydrogen (5 Psi) was conducted in. The reaction vessel was shaked overnight and filtered through Celite. The filtrant was concentrated and purified by SiO₂ chromatography (DCM/MeOH/Et₃N=4/1/0.05) to give the title compound (1.66 g, 86% yield) as a off-white foam. MS ESI m/z calcd for C₁₃H₁₇N₂O₃ [M+H]⁺ 249.12, found 249.50.

### Example 321. Synthesis of 4-((14S,17S)-1-azido-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl 8-hydroxy-7-methoxy-5-oxo-2,3,11,11a-tetrahydro-1H-benzo[e]pyrrolo[1,2-a][1,4]diazepine-10(5H)-carboxylate

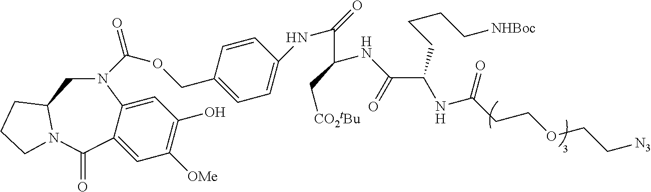

To a solution of (14S,17S)-tert-butyl 1-azido-14-(4-((tert-butoxycarbonyl)amino)butyl)-17-((4-(hydroxymethyl)phenyl)carbamoyl)-12,15-dioxo-3,6,9-trioxa-13,16-diazanonadecan-19-oate (10.15 g, 13.50 mmol) in dry THF (300 mL) was added DIPEA (3.15 g, 24.41 mmol) and a solution of triphosgene (5.15 g, 17.36 mmol) in dry THE (50 mL) at 4-8° C. After 15 min of stirring, the solution was recooled to 4-8° C. and then added dropwise to a solution of 8-hydroxy-7-methoxy-2,3,11,11a-tetrahydro-1H-benzo[e]pyrrolo[1,2-a][1,4]diazepin-5(10H)-one (2.92 g, 11.76 mmol) in mixture of THF (100 mL) at 4-8° C. in 45 min. The resultant solution was allowed to warm to room temperature and stirred overnight. The mixture was diluted with toluene (50 ml), evaporated in vacuo and purified by SiO₂ chromatography (DCM/MeOH=15/1) to give the title compound (10.02 g, 82% yield) as a yellow foam. MS ESI m/z calcd for C₅₀H₇₄N₉O₁₅ [M+H]⁺ 1040.52, found 1040.90.

### Example 322. Synthesis of (S)-4-((14S,17S)-1-azido-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl 8-(3-iodopropoxy)-7-methoxy-5-oxo-2,3,11,11a-tetrahydro-1H-benzo[e]pyrrolo[1,2-a][1,4]diazepine-10(5H)-carboxylate

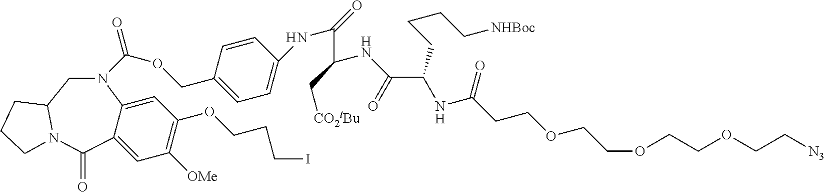 [SMILES: COc1cc2c(cc1OCCCI)N(C(=O)OCc1ccc(NC(=O)[C@H](CC(=O)OC(C)(C)C)NC(=O)[C@H](CCCCNC(=O)OC(C)(C)C)NC(=O)CCOCCOCCOCCN=[N+]=[N-])cc1)CC1CCCN1C2=O]

To a solution of4-((14S,17S)-1-azido-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl 8-hydroxy-7-methoxy-5-oxo-2,3,11,11a-tetrahydro-1H-benzo[e]pyrrolo[1,2-a][1,4]diazepine-10(5H)-carboxylate (2.02 g, 1.94 mmol) in butanone (50 ml) was added Cs₂CO₃ (2.50 g, 7.67 mmol) and 1,3-diiodopropane (2.50 g, 8.45 mmol). The mixture was stirred at 45° C. under dark for 36 h, concentrated and purified on SiO₂ column eluted with EtOAc/CH₂Cl₂ (1:5) to afford the title compound (2.08 g, 90% yield). MS ESI m/z calcd for C₅₂H₇₇IN₉O₁₅ [M+H]⁺ 1194.45, found 1194.95.

### Example 323. Synthesis of (S)-2-((S)-1-azido-14-methyl-12-oxo-3,6,9-trioxa-13-azapentadecanamido)-N-(4-(hydroxymethyl)phenyl)propanamide

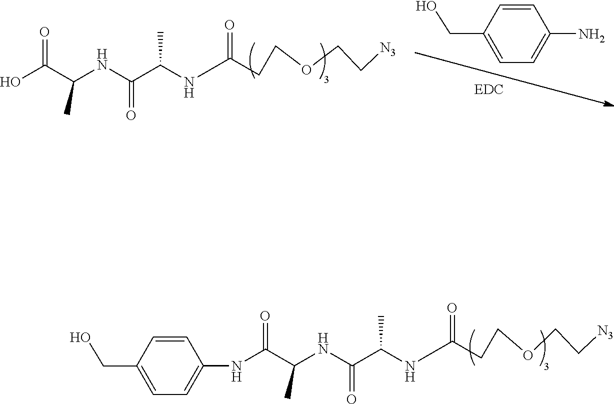

To a solution of (14S,17S)-1-azido-14,17-dimethyl-12,15-dioxo-3,6,9-trioxa-13,16-diazaoctadecan-18-oic acid (3.02 g, 7.75 mmol) and (4-aminophenyl)methanol (1.05 g, 8.53 mmol) in DMA was added EDC (4.90 g, 25.52 mmol). The mixture was stirred at RT for 14 h, concentrated and purified on SiO₂ column eluted with EtOAc/CH₂Cl₂ (1:8 to 1:3) to afford the title compound (3.52 g, 92% yield). MS ESI m/z calcd for C₂₂H₃₅IN₆O₇[M+H]⁺ 495.25, found 495.60.

### Example 324. Synthesis of (11R,11aS)-4-((14S,17S)-1-azido-14,17-dimethyl-12,15-dioxo-3,6,9-trioxa-13,16-diazaoctadecanamido)benzyl 8-(benzyloxy)-11-hydroxy-7-methoxy-2-methylene-5-oxo-2,3,11,11a-tetrahydro-1H-benzo[e]pyrrolo[1,2-a][1,4]diazepine-10(5H)-carboxylate

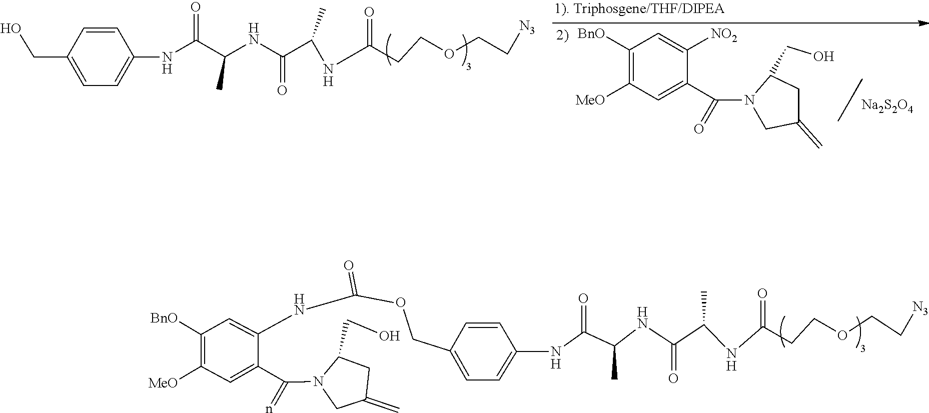

A mixture of (S)-(4-(benzyloxy)-5-methoxy-2-nitrophenyl)(2-(hydroxymethyl)-4-methylene-pyrrolidin-1-yl)methanone (3.90 g, 9.80 mmol) and Na₂S₂O₄ (6.0 g, 34.47 mmol) in THE (60 ml) and H₂O (40 ml) was stirred at room temperature for 20 h, adjusted pH to 10 with Na₂CO₃, concentrated, purified on C-18 short column eluted with H₂O/MeOH/Et₃N (from 99.4/0.5/0.2 to 50/49.8/0.2). The fractions containing the reduced amino product were pooled, concentrated, diluted with THE (50 ml), then cooled to 4-8° C. Separately to a solution of 2-(1-azido-14-methyl-12-oxo-3,6,9-trioxa-13-azapentadecanamido)-N-(4-(hydroxy-methyl)phenyl)-propanamide (6.70 g, 13.56 mmol) in dry THF (150 mL) was added DIPEA (3.50 g, 27.12 mmol) and a solution of triphosgene (4.10 g, 13.80 mmol) in dry THE (20 mL) at 4-8° C. After 15 min of stirring at 4-8° C., the solution was added dropwise to the above amino solution at 4-8° C. for 45 min. The mixture was warmed to RT and continued to stir for 2 h, concentrated, extracted with CH₂Cl₂ (3×30 ml), dried over Na₂SO₄, evaporated and purified on SiO₂ column eluted with EtOAc/CH₂Cl₂ (1:10 to 1:5) to afford the title compound (7.23 g, 83% yield in two steps). MS ESI m/z calcd for C₄₅H₅₇IN₈O₁₂ [M+H]⁺ 889.40, found 889.90.

### Example 325. Synthesis of (11S,11aS)-4-((14S,17S)-1-azido-14,17-dimethyl-12,15-dioxo-3,6,9-trioxa-13,16-diazaoctadecanamido)benzyl 8-(benzyloxy)-11-hydroxy-7-methoxy-2-methylene-5-oxo-2,3,11,11a-tetrahydro-1H-benzo[e]pyrrolo[1,2-a][1,4]diazepine-10(5H)-carboxylate

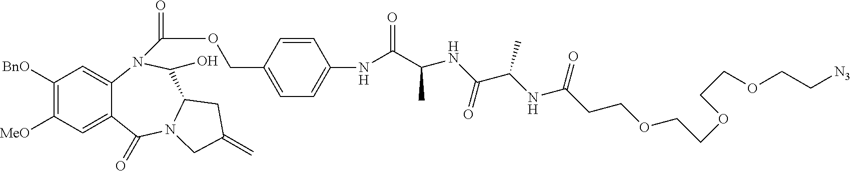

To a solution of (11R,11aS)-4-((14S,17S)-1-azido-14,17-dimethyl-12,15-dioxo-3,6,9-trioxa-13,16-diazaoctadecanamido)benzyl 8-(benzyloxy)-11-hydroxy-7-methoxy-2-methylene-5-oxo-2,3,11,11a-tetrahydro-1H-benzo[e]pyrrolo[1,2-a][1,4]diazepine-10(5H)-carboxylate (3.80 g, 4.27 mmol) in dry DCM (40 mL) was added Dess-Martin periodinane (DMP) (2.80 g, 6.60 mmol) under nitrogen at room temperature. After completion of conversion, the reaction solution was added aqueous Na2SO3 and followed by aqueous NaHCO₃, the mixture was stirred for further 15 minutes and extracted with DCM (3×20 mL). The combined organic extract was washed with brine, dried, filtered and concentrated. The residue was purified by SiO₂ chromatography (DCM/EtOAc=5/1 to 2:1) to give the title compound (2.99 g, 79% yield) as an off-white foam. MS ESI m/z calcd for C₄₄H₅₅N₈O₁₂ [M+H]⁺ 886.39, found 886.80.

### Example 326. Synthesis of (11S,11aS)-4-((14S,17S)-1-azido-14,17-dimethyl-12,15-dioxo-3,6,9-trioxa-13,16-diazaoctadecanamido)benzyl 8,11-dihydroxy-7-methoxy-2-methylene-5-oxo-2,3,11,11a-tetrahydro-1H-benzo[e]pyrrolo[1,2-a][1,4]diazepine-10(5H)-carboxylate

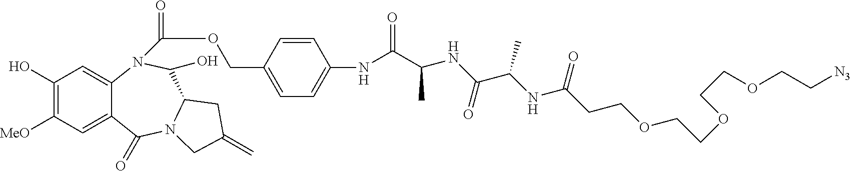

To a solution of (11S,11aS)-4-((14S,17S)-1-azido-14,17-dimethyl-12,15-dioxo-3,6,9-trioxa-13,16-diazaoctadecanamido)benzyl 8-(benzyloxy)-11-hydroxy-7-methoxy-2-methylene-5-oxo-2,3,11,11a-tetrahydro-1H-benzo[e]pyrrolo[1,2-a][1,4]diazepine-10(5H)-carboxylate (2.90 g, 3.27 mmol) in 40 ml of CH₂C12 was added 15 ml of CH₃SO₃H at 0° C. The mixture was stirred at 0° C. for 10 min then r.t. for 1 h, diluted with CH₂Cl₂, pH adjusted with cold 1.0 N NaHCO₃ to 4 and filtered. The aqueous layer was extracted with CH₂Cl₂(3×60 ml). The organic layers were combined, dried over Na₂SO₄, filtered, evaporated and purified on SiO₂ column chromatography (CH₃OH/CH₂Cl₂ 1:15 to 1:5) to afford 1.95 g (75% yield) of the title product. MS ESI m/z calcd for C₃₇H₄₈IN₈O₁₂ [M+H]⁺ 797.34, found 797.90.

### Example 327. Synthesis of (11S,11aS)-4-((14S,17S)-1-azido-14,17-dimethyl-12,15-dioxo-3,6,9-trioxa-13,16-diazaoctadecanamido)benzyl 8-(3-(((S)-10-(((4-((14S,17S)-1-azido-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate

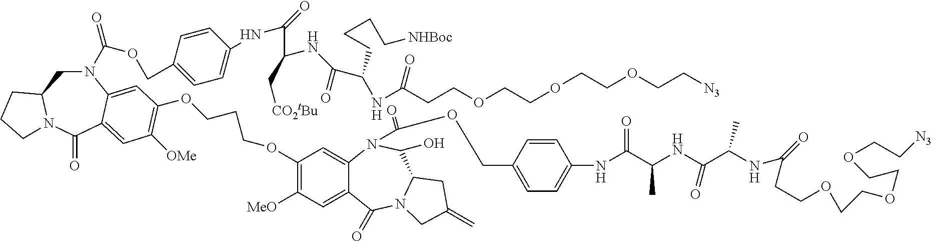

To a solution of (11S,11aS)-4-((14S,17S)-1-azido-14,17-dimethyl-12,15-dioxo-3,6,9-trioxa-13,16-diazaoctadecanamido)benzyl 8,11-dihydroxy-7-methoxy-2-methylene-5-oxo-2,3,11,11a-tetrahydro-1H-benzo[e]pyrrolo[1,2-a][1,4]diazepine-10(5H)-carboxylate (402 mg, 0.504 mmol) and (S)-4-((14S,17S)-1-azido-17-(2-(tert-butoxy)-2-oxoethyl)-14-(4-((tert-butoxycarbonyl)amino)-butyl)-12,15-dioxo-3,6,9-trioxa-13,16-diazaoctadecanamido)benzyl 8-(3-iodopropoxy)-7-methoxy-5-oxo-2,3,11,11a-tetrahydro-1H-benzo[e]pyrrolo[1,2-a][1,4]diazepine-10(5H)-carboxylate (650 mg, 0.544 mmol) in butanone (50 ml) was added Cs₂CO₃ (0.50 g, 1.53 mmol). The mixture was stirred at 45° C. under dark for 36 h, concentrated and purified on SiO₂ column eluted with EtOAc/CH₂Cl₂ (1:8 to 1:3) to afford the title compound (809 mg, 86% yield). MS ESI m/z calcd for C₈₉H₁₂₄N₁₇O₂₇ [M+H]⁺ 1862.89, found 1863.45.

### Example 328. Synthesis of (11S,11aS)-4-((14S,17S)-1-amino-14,17-dimethyl-12,15-dioxo-3,6,9-trioxa-13,16-diazaoctadecanamido)benzyl 8-(3-(((S)-10-(((4-((14S,17S)-1-amino-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate

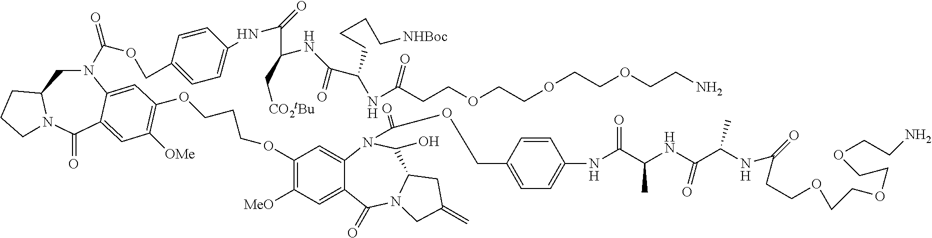

(11S,11 aS)-4-((14S,17S)-1-Azido-14,17-dimethyl-12,15-dioxo-3,6,9-trioxa-13,16-diazaoctadecanamido)benzyl 8-(3-(((S)-10-(((4-((14S,17S)-1-azido-17-(2-(tert-butoxy)-2-oxoethyl)-14-(4-((tert-butoxycarbonyl)amino)butyl)-12,15-dioxo-3,6,9-trioxa-13,16-diazaoctadecanamido)-benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate (750 mg, 0.402 mmol) in THE (8 ml) was added Me₃P (1.0 M in toluene, 2.0 ml, 2.0 mmol) at 0-4° C. under N₂. After stirred for 5 min, the ice bath was removed and the reaction mixture was stirred at RT for 2 h. Then, water (1 ml) was added and the mixture was stirred for 10 min. The mixture was diluted with 1,4-dioxane (10 ml), concentrated and co-evaporated with dioxane/toluene to dryness to yield the crude amino product (725 mg, -99% yield) which was used directly for next step without further purification. MS ESI m/z calcd for C₈₉H₁₂₈N₁₃O₂₇ [M+H]⁺ 1810.90, found 1811.50.

### Example 329. Synthesis of an Asymmetrically Cross-Linked PBD Dimer C-09

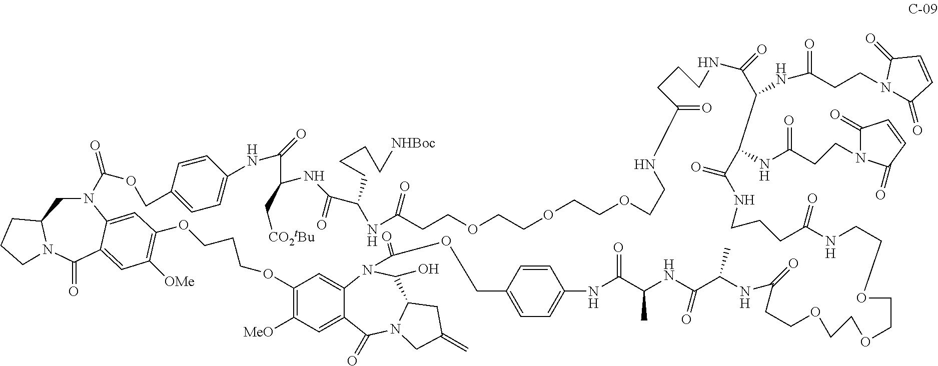

To the above crude amino compound ((11S,11aS)-4-((14S,17S)-1-amino-14,17-dimethyl-12,15-dioxo-3,6,9-trioxa-13,16-diazaoctadecanamido)benzyl 8-(3-(((S)-10-(((4-((14S,17S)-1-amino-17-(2-(tert-butoxy)-2-oxoethyl)-14-(4-((tert-butoxycarbonyl)amino)butyl)-12,15-dioxo-3,6,9-trioxa-13,16-diazaoctadecanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)propoxy)-11-hydroxy-7-methoxy-2-methylene-5-oxo-2,3,11,11a-tetrahydro-1H-benzo[e]pyrrolo[1,2-a][1,4]diazepine-10(5H)-carboxylate) in dry DMA (8 ml) was added 4,4′-(((2R,3S)-2,3-bis(3-(2,5-dioxo-2,5-dihydro-1H-pyrrol-1-yl)propanamido)succinyl)bis(azanediyl))dibutanoic acid (248.0 mg, 0.400 mmol) and EDC (500.0 mg, 2.60 mmol). The mixture was stirred for 24 h, concentrated and purified on C₁₈ preparative HPLC (17% C18, 250 mm×50 mm), eluted with water/CH₃CN (from 800% water to 300% water in 40 min, 9 ml/min) to afford 488.1 mg (51% yield) of the C-9 product after drying under high vacuum. ESI MS m/z C₁₁₅H₁₅₆N₁₉O₃₇ [M+H]⁺, cacld. 2395.08, found 2395.90.

### Example 330. Synthesis of an Unsymmetrically Cross-Linked PBD Dimer C-10

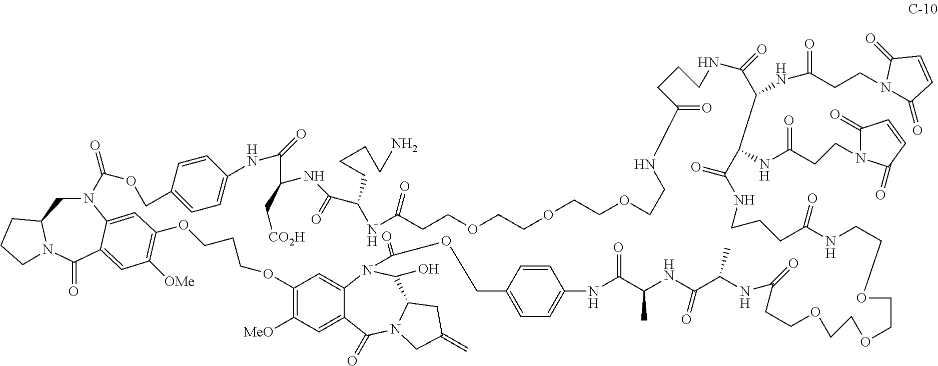

C-09 compound (465.0 mg, 0.194 mmol) was dissolved in DCM (4 m followed by addition of TFA (2 ml) at 0-4° C. The reaction mixture was then stirred at RT for 1 h, diluted with toluene (5 ml), then concentrated, and co-evaporated with DCM/toluene to dryness to afford the crude product C-3 (48.0 mg, 100% yield, 92% pure by HPLC) which was further purified by reverse phase HPLC (250 (L) mm x 20(d) mm, C₁₈ column, 5-60% acetonitrile/water in 40 min, v=8 ml/min) to afford the pure product C-10 (373.1 mg, 85% yield, 96% pure) as a foam. ESI MS m/z: calcd for C₁₀₆H₁₄₀N₁₉O₃₅ [M+H]⁺ 2238.97, found 2239.50.

### Example 331. Synthesis of an Asymmetrically Cross-Linked PBD Dimer C-11

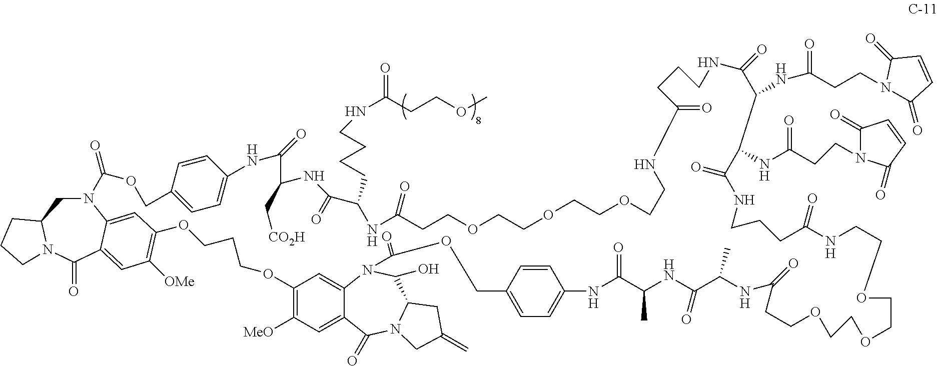

C-10 compound (235.0 mg, 0.105 mmol) was dissolved in a mixture solution of THF (3 ml) and 0.1 M, NaH₂PO₄ (3 ml), pH 7.5, followed by addition of N-succinimidyl 2,5,8,11,14,17,20,23-octaoxahexacosan-26-oate (43.0 mg, 0.084 mmol) in 4 portions in 2 h. The reaction mixture was then continued to stir at RT for 4 h, and co-evaporated with DMF (10 ml) to dryness to afford the crude product C-11 which was further purified by reverse phase HPLC (250 (L) mm x 50(d) mm, C₁₈ column, 20-60% acetonitrile/water in 40 min, v=8 ml/min) to afford the pure product C-11 (215.5 mg, 78% yield, 95% pure) as a foam. ESI MS m/z: calcd for C₁₂₄H₁₇₄N₁₉O₄₄ [M+H]⁺ 2633.20, found 2633.85.

### Example 332. Synthesis of an Asymmetrically Cross-Linked PBD Dimer C-12

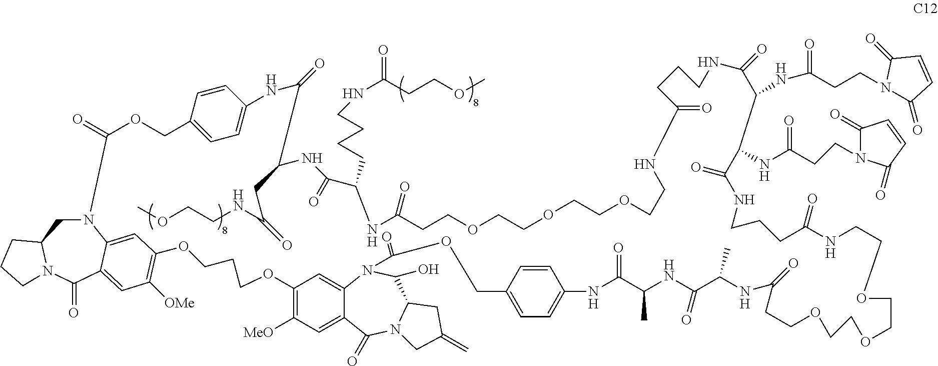

To a solution of C-11 compound (65.0 mg, 0.0246 mmol) and 2,5,8,11,14,17,20,23-octaoxapentacosan-25-amine (15.1 mg, 0.0394 mmol) in dry DMA (2 ml) was added EDC (30.0 mg, 0.156 mmol). The reaction mixture was stirred at RT for 15 h, concentrated, purified by reverse phase HPLC (250 (L) mm x 30 (d) mm, C₁s column, 20-60% acetonitrile/water in 40 min, v=8 ml/min) to afford the pure product C-12 (60.2 mg, 81% yield, 95% pure by HPLC) as a foam. ESI MS m/z: calcd for C₁₄₁H₂₀₉N₂₀O₅[M+H]⁺ 2998.43, found 2999.40.

### Example 333. Synthesis of Nitro-α-Amanitin

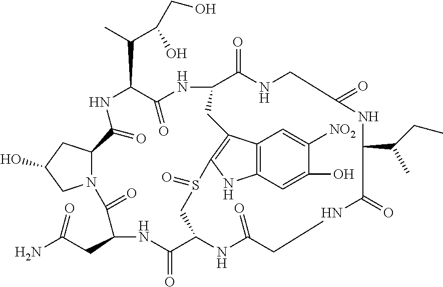 [SMILES: CC[C@H](C)[C@H]1NC(=O)CNC(=O)[C@@H]2Cc3c([nH]c4cc(O)c([N+](=O)[O-])cc34)S(=O)C[C@H](NC(=O)CNC1=O)C(=O)N[C@@H](CC(N)=O)C(=O)N1C[C@H](O)C[C@H]1C(=O)N[C@@H](C(C)[C@@H](O)CO)C(=O)N2]

To a solution of a-amanitin (15.0 mg, 0.0163 mmol, PCT/IB2016/052246) in acetic acid (0.5 mL) and CH₂C₂(1 mL) was added 70% HNO₃ (0.3 mL) at 0° C. The reaction was stirred at 0° C. for 1 h then room temperature 2 h. After water (5 mL) and DMA (4 ml) were, the reaction mixture was concentrated and purified by prep-HPLC (H₂O/MeCN) to give a light yellow solid (9.8 mg, 62% yield). ESI MS m/z: calcd for C₃₉H₅₄N₁₁O₁₆S [M+H]⁺ 963.34, found 964.95.

### Example 334. Synthesis of Nitro-α-Amanitin

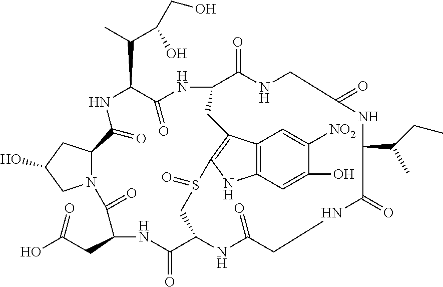 [SMILES: CC[C@H](C)[C@H]1NC(=O)CNC(=O)[C@@H]2Cc3c([nH]c4cc(O)c([N+](=O)[O-])cc34)S(=O)C[C@H](NC(=O)CNC1=O)C(=O)N[C@@H](CC(=O)O)C(=O)N1C[C@H](O)C[C@H]1C(=O)N[C@@H](C(C)[C@@H](O)CO)C(=O)N2]

To a solution of P-amanitin (15.0 mg, 0.0163 mmol, PCT/IB2016/052246) in acetic acid (0.5 mL) and CH₂Cl₂ (1 mL) was added 70% HNO₃ (0.3 mL) at 0° C. The reaction was stirred at 0° C. for 1 h then room temperature 2 h. After water (5 mL) and DMA (4 ml) were added, the reaction mixture was concentrated and purified by prep-HPLC (H₂O/MeCN) to give a light yellow solid (9.8 mg, 62% yield). ESI MS m/z: calcd for C₃₉H₅₃N₁₀O₁₇S [M+H]⁺ 965.32, found 965.86.

### Example 335. Synthesis of a Conjugatable a-Amanitin Analogs, D-01 and D-02

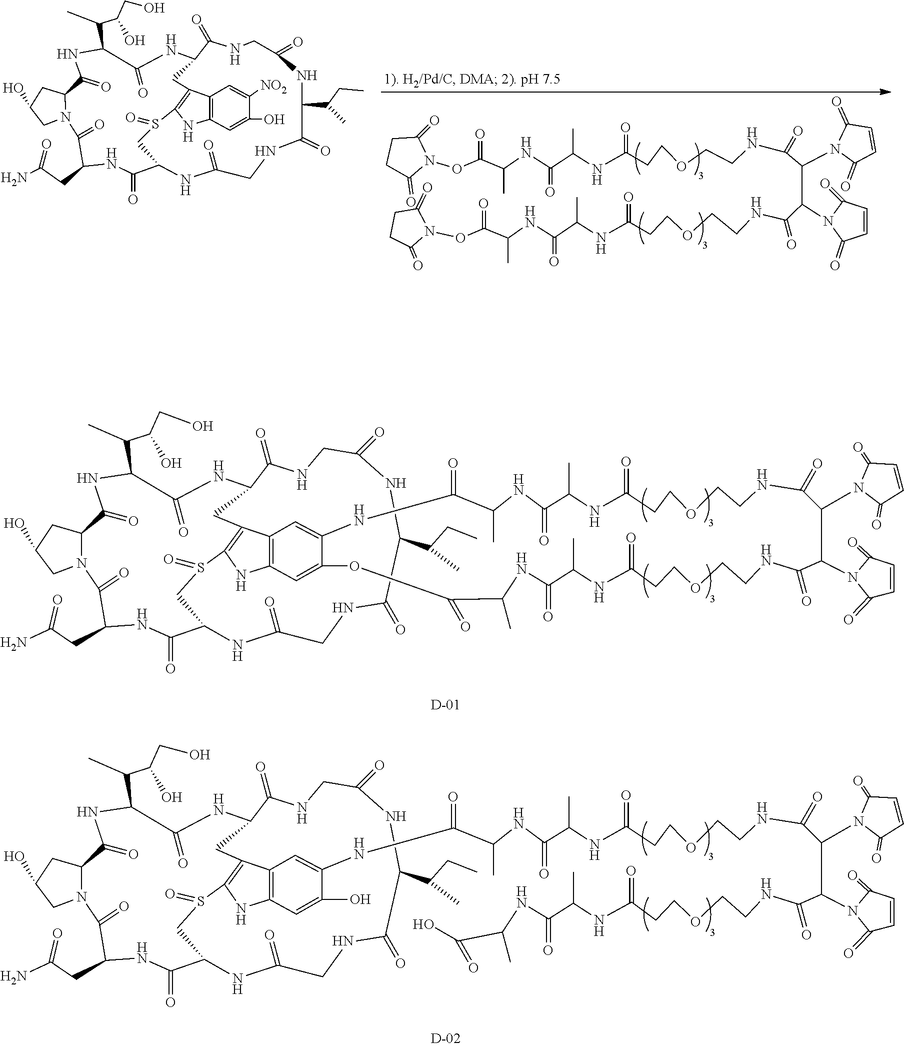

To a solution of nitro-ax-amanitin (9.0 mg, 0.0093 mmol) in DMA (1 ml)) was added Pd/C (3 mg, 50% wet), then hydrogenated (1 atm) at room temperature for 6 h. The catalyst was filtered off, followed by addition of 0.5 ml, 0.1 M NaH2PO4, pH 7.5 and bis(2,5-dioxopyrrolidin-1-yl) 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioate (11.0 mg, 0.0092 mmol). The mixture was stirred at r.t. overnight, concentrated, and purified on C₁₈ preparative HPLC with elution of water/CH₃CN (from 95% water to 25% water in 45 min). The fractions of HPLC collection containing each of product were pooled, concentrated and dried under high vacuum to afford product D-01 (6.1 mg, 35% yield), ESI MS m/z C₈₁H₁₁₄N₁₉O₃₂S [M+H]⁺, cacld. 1896.75, found 1897.20; and product D-02 (4.9 mg, 27% yield), ESI MS m/z C₈₁H₁₁₆N₁₉O₃₃S [M+H]⁺, cacld. 1914.76, found 1914.40.

### Example 336. Synthesis of a Conjugatable β-Amanitin Analogs D-03 and D-04

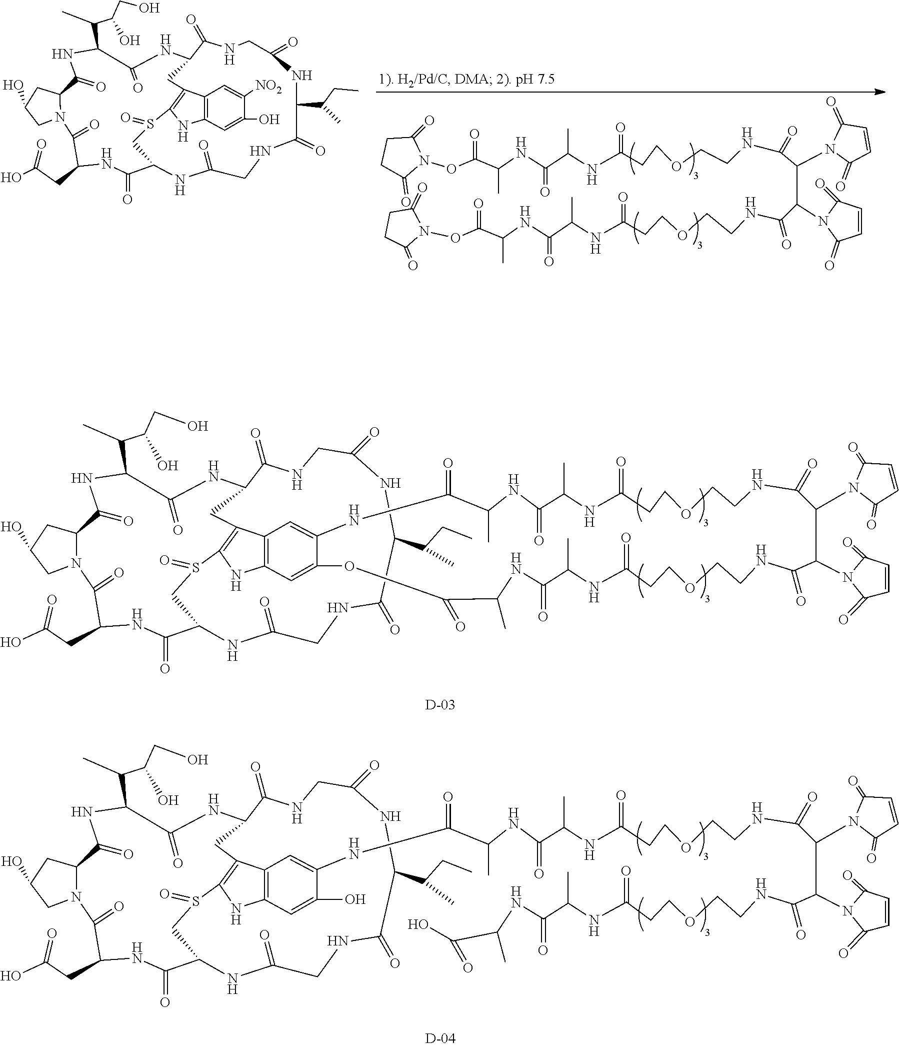

To a solution of nitro-β-amanitin (9.0 mg, 0.0093 mmol) in DMA (1 ml)) was added Pd/C (3 mg, 50% wet), then hydrogenate (1 atm) at room temperature for 6 h. The catalyst was filtered off, followed by addition of 0.5 ml, 0.1 M NaH2PO4, pH 7.5 and bis(2,5-dioxopyrrolidin-1-yl) 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioate (11.0 mg, 0.0092 mmol). The mixture was stirred at r.t. overnight, concentrated, and purified on C₁₈ preparative HPLC with elution of water/CH₃CN (from 95% water to 25% water in 45 min). The fractions of HPLC collection containing each of product were pooled, concentrated and dried under high vacuum to afford product D-03 (7.0 mg, 40% yield), ESI MS m/z C₈₁H₁₁₃N₁₈O₃₃S [M+H]⁺, cacld. 1896.74, found 1897.20; and product D-04 (4.7 mg, 25% yield), ESI MS m/z C₈₁H₁₁₅N₁₈O₃₄S [M+H]⁺, cacld. 1915.75, found 1916.30.

### Example 337. Synthesis of a Conjugatable α-Amanitin Analog (D-05) Having a Bis-Linker

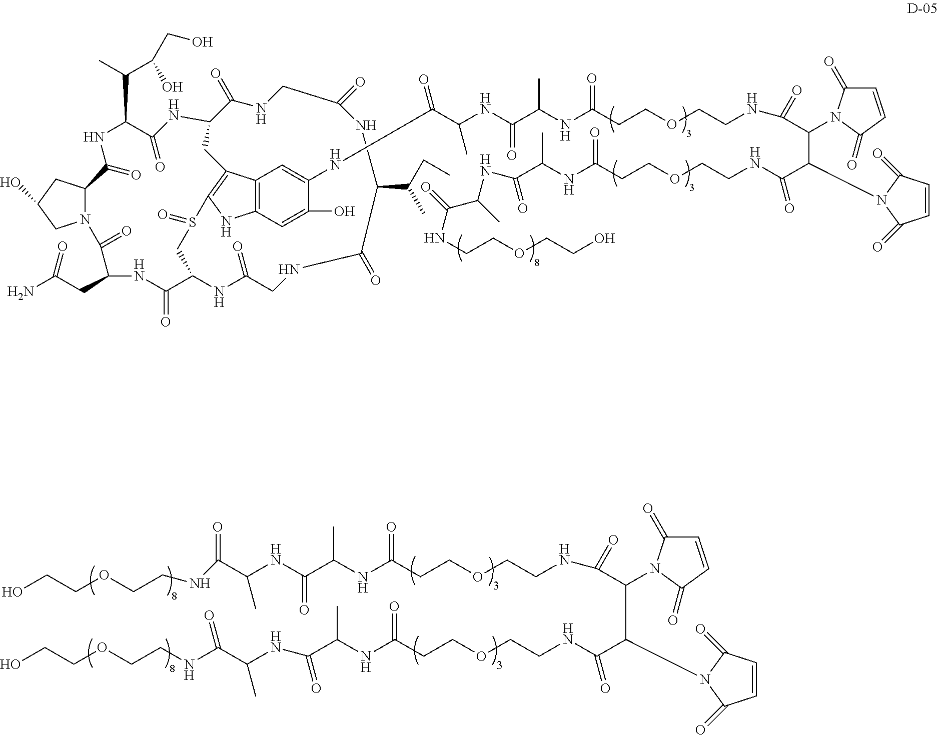

To a solution of nitro-α-amanitin (9.0 mg, 0.0093 mmol) in dry DMA (1 ml)) was added P-423,C1 Pd/C (3 mg, 50% wet), then hydrogenated (1 atm) at room temperature for 6 h. The catalyst was filtered off, washed with DMA (1 ml) and then bis(2,5-dioxopyrrolidin-1-yl) 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioate (40.0 mg, 0.033 mmol) and DIPEA (2 μl, 0.011 mmol). The mixture was stirred at RT for 4h. followed by addition of 26-amino-3,6,9,12,15,18,21,24-octaoxahexacosan-1-ol (30.0 mg, 0.072 mmol). The mixture was continued to stir overnight, concentrated and purified on C₁₈ preparative HPLC, eluted with water/CH₃CN (from 95% water to 30% water in 45 min) to afford (14.5 mg 69% yield) of the title product D-05 after drying under high vacuum. ESI MS m/z C₉₉H₁₅₃N₂₀O₄₁S [M+H]⁺, cacld. 2310.01, found 2310.90; and the side product, 2,3-bis(2,5-dioxo-2,5-dihydro-1H-pyrrol-1-yl)-N1,N4-bis(1-hydroxy-29,32-dimethyl-28,31,34-trioxo-3,6,9,12,15,18,21,24,37,40,43-undecaoxa-27,30,33-triazapentatetracontan-45-yl)succinamide (24.3 mg, 0.013 mmol), ESI MS m/z C₇₈H₁₃₆N₁₀O₃₆ [M+H]⁺, cacld. 1789.91, found 1790.20.

### Example 338. Synthesis of a Conjugatable β-Amanitin Analog (D-06) Having a Bis-Linker

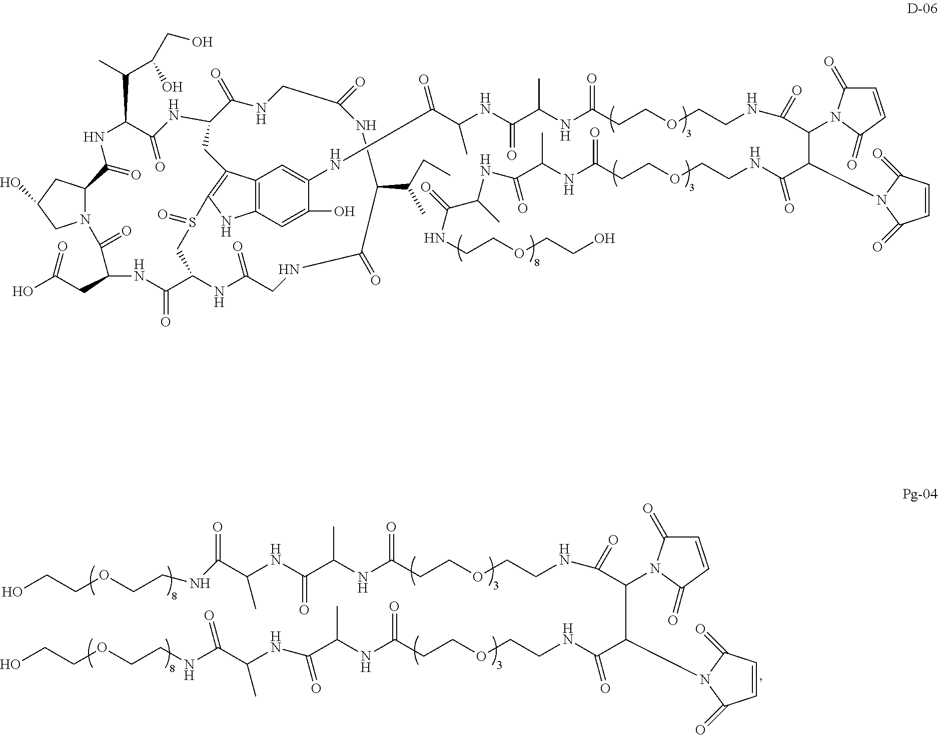

To a solution of nitro-o-amanitin (9.0 mg, 0.0093 mmol) in dry DMA (1 ml)) was added Pd/C (3 mg, 50% wet), then hydrogenated (1 atm) at room temperature for 6 h. The catalyst was filtered off, washed with DMA (1 ml) and then bis(2,5-dioxopyrrolidin-1-yl) 21,22-bis(2,5-dioxo-2,5-dihydro-1H-pyrrol-1-yl)-2,5,38,41-tetramethyl-4,7,20,23,36,39-hexaoxo-10,13,16,27,30,33-hexaoxa-3,6,19,24,37,40-hexaazadotetracontane-1,42-dioate (40.0 mg, 0.033 mmol) and DIPEA (2 μl, 0.011 mmol). The mixture was stirred at RT for 4h. followed by addition of 26-amino-3,6,9,12,15,18,21,24-octaoxahexacosan-1-ol (30.0 mg, 0.072 mmol). The mixture was continued to stir overnight, concentrated and purified on C₁₈ preparative HPLC, eluted with water/CH₃CN (from 95% water to 30% water in 45 min) to afford (14.9 mg 69% yield) of the title product D-06 after drying under high vacuum. ESI MS m/z C₉₉H₁₅₂N₁₉O₄₂S [M+H]⁺, cacld. 2311.00, found 2311.90; and the side product Pg-04, 2,3-bis(2,5-dioxo-2,5-dihydro-1H-pyrrol-1-yl)-N1,N4-bis(1-hydroxy-29,32-dimethyl-28,31,34-trioxo-3,6,9,12,15,18,21,24,37,40,43-undecaoxa-27,30,33-triazapentatetracontan-45-yl)succinamide (24.8 mg, 0.013 mmol), ESI MS m/z C₇₈H₁₃₆N₁₀O₃₆ [M+H]⁺, cacld. 1789.91, found 1790.20.

### Example 339. General Method of Preparation of Conjugate

To a mixture of 2.0 mL of 10 mg/ml a her2 antibody in pH 6.0-8.0, were added of 0.70˜2.0 mL PBS buffer of 100 mM NaH₂PO₄, pH 6.5˜8.5 buffers, TCEP (16-20 μL, 20 mM in water) and the compound A-01, A-02, A-03, A-04, B-01, B-02, B-03, B-04, B-05, B-06, B-07, B-08, B-09, B-10, B-11, B-12, B-13, B-14, B-15, B-16, C-02, C-03, C-04, C-05, C-06, C-07, C-08, C-09, C-10, C-11, C-12, D-01, D-02, D-03, D-04, D-05 or D-06 (28-32 μL, 20 mM in DMA) independently. The mixture was incubated at RT for 4-18 h, then DHAA (135 μL, 50 mM) was added in. After continuous incubation at RT overnight, the mixture was purified on G-25 column eluted with 100 mM NaH₂PO₄, 50 mM NaCl pH 6.0-7.5 buffer to afford 12.8-18.1 mg of the conjugate compound Aa-01, Aa-02, Aa-03, Aa-04, Ba-01, Ba-02, Ba-03, Ba-04, Ba-05, Ba-06, Ba-07, Ba-08, Ba-09, Ba-10, Ba-11, Ba-12, Ba-13, Ba-14, Ba-15, Ba-16, Ca-02, Ca-03, Ca-04, Ca-05, Ca-06, Ca-07, Ca-08, Ca-09, Ca-10, Ca-11, Ca-12, Da-01, Da-02, Da-03, Da-04, Da-05 or Da-06 (75%˜90% yield) accordingly in 14.4-15.5 ml buffer. The drug/antibody ratio (DAR) was 3.1-4.2 for conjugate which was determined via UPLC-QTOF mass spectrum. It was 94˜99% monomer analyzed by SEC HPLC (Tosoh Bioscience, Tskgel G3000SW, 7.8 mm ID x 30 cm, 0.5 ml/min, 100 min) and a single band measured by SDS-PAGE gel. The conjugate structures are displayed below:

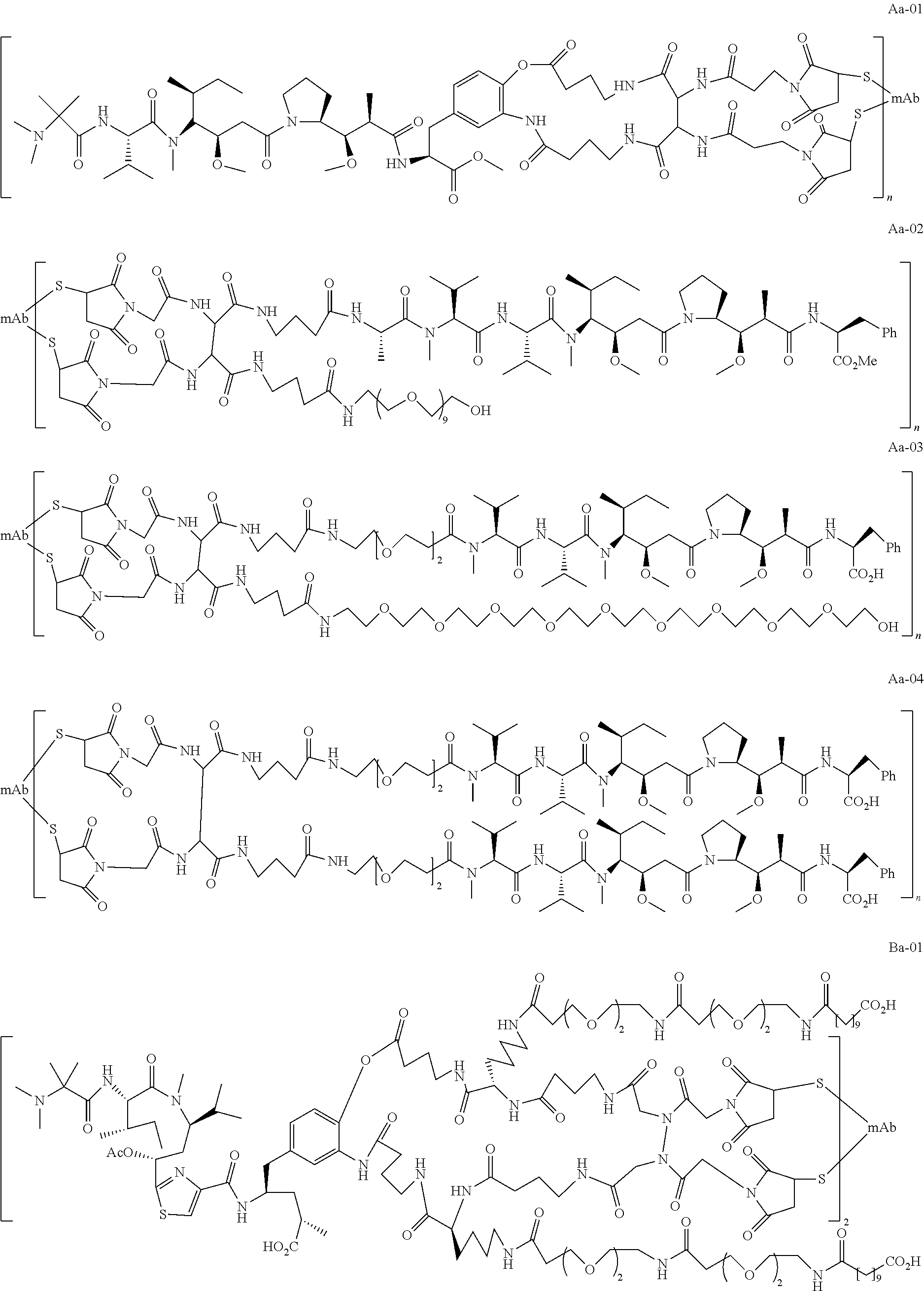

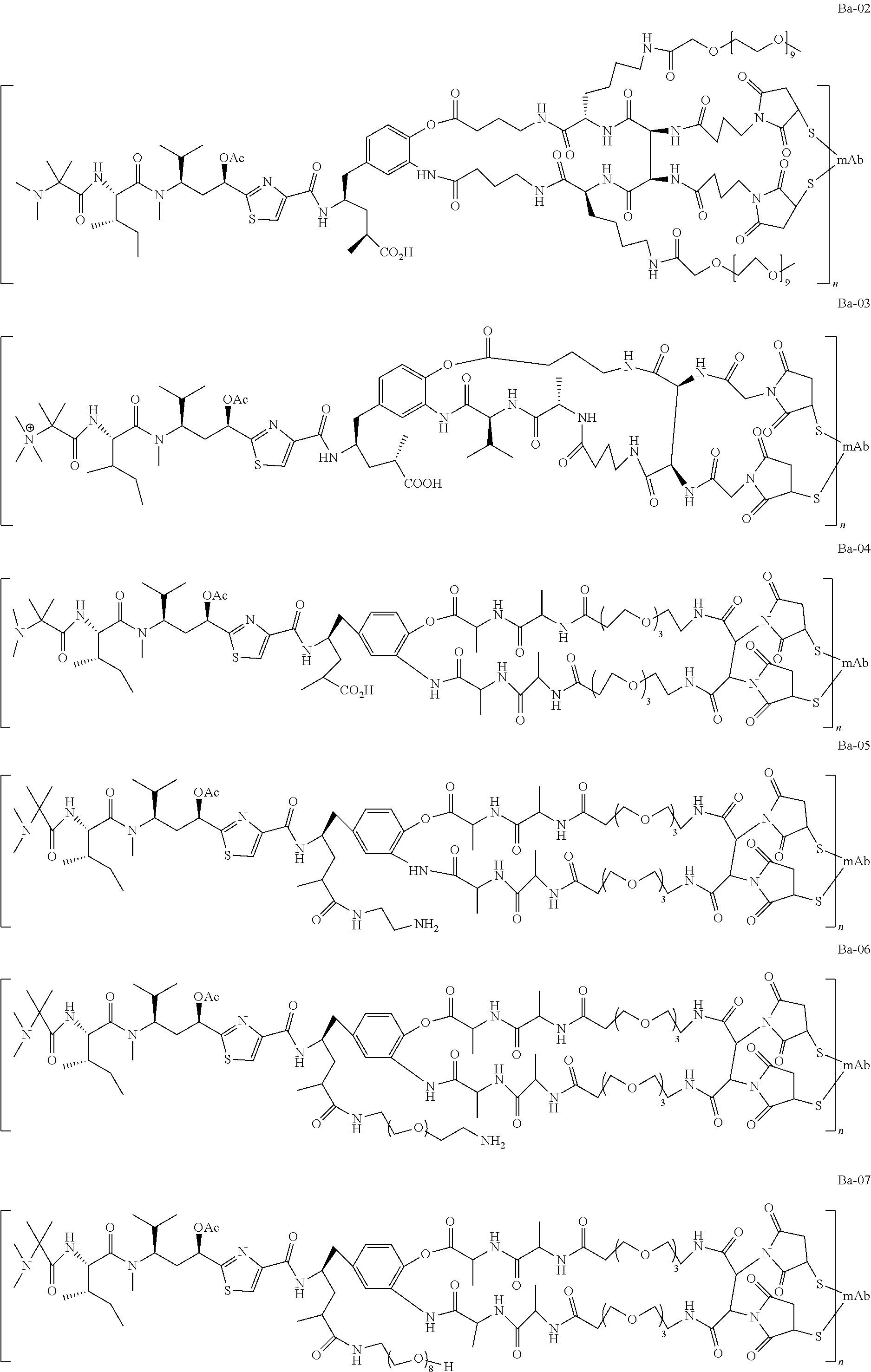

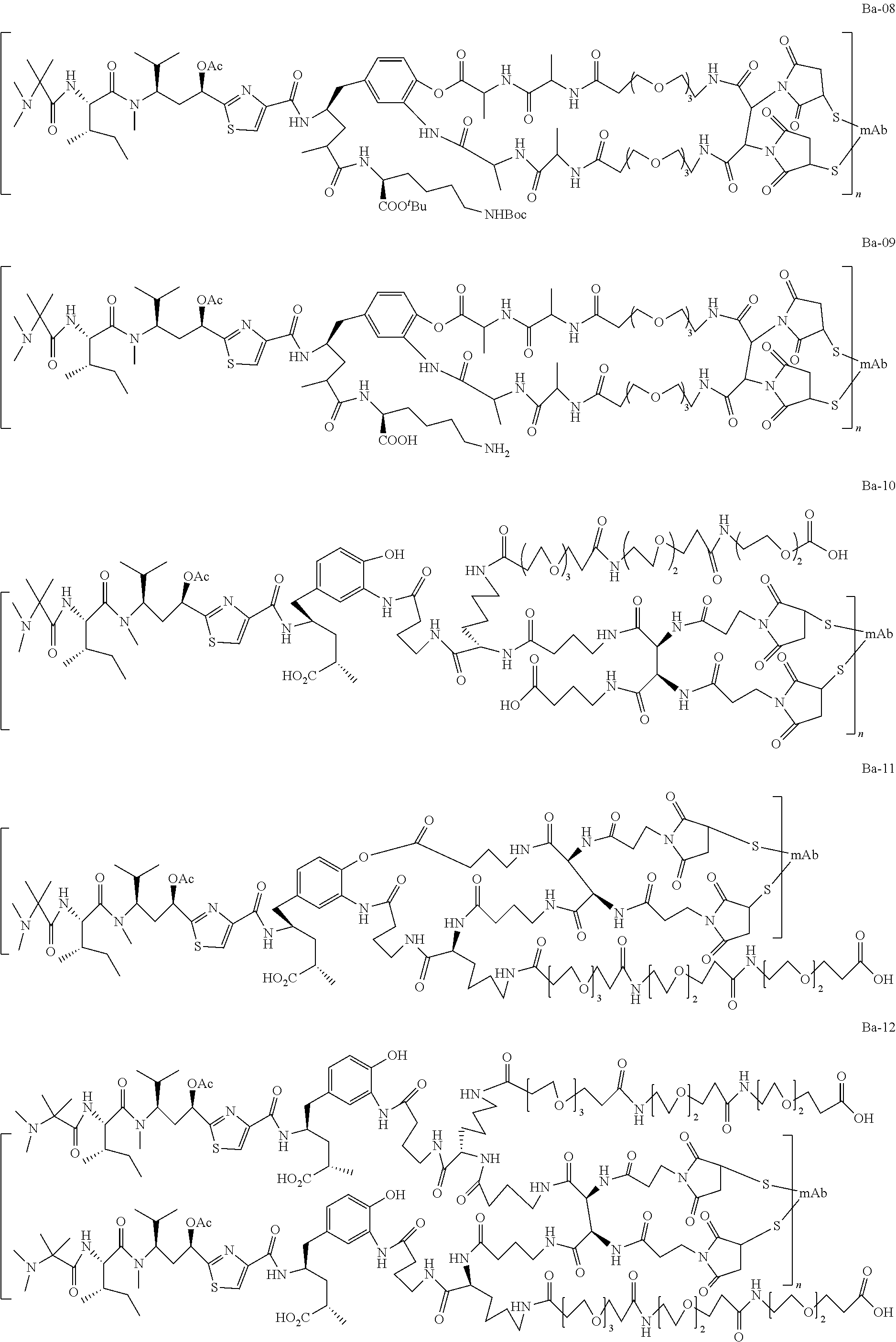

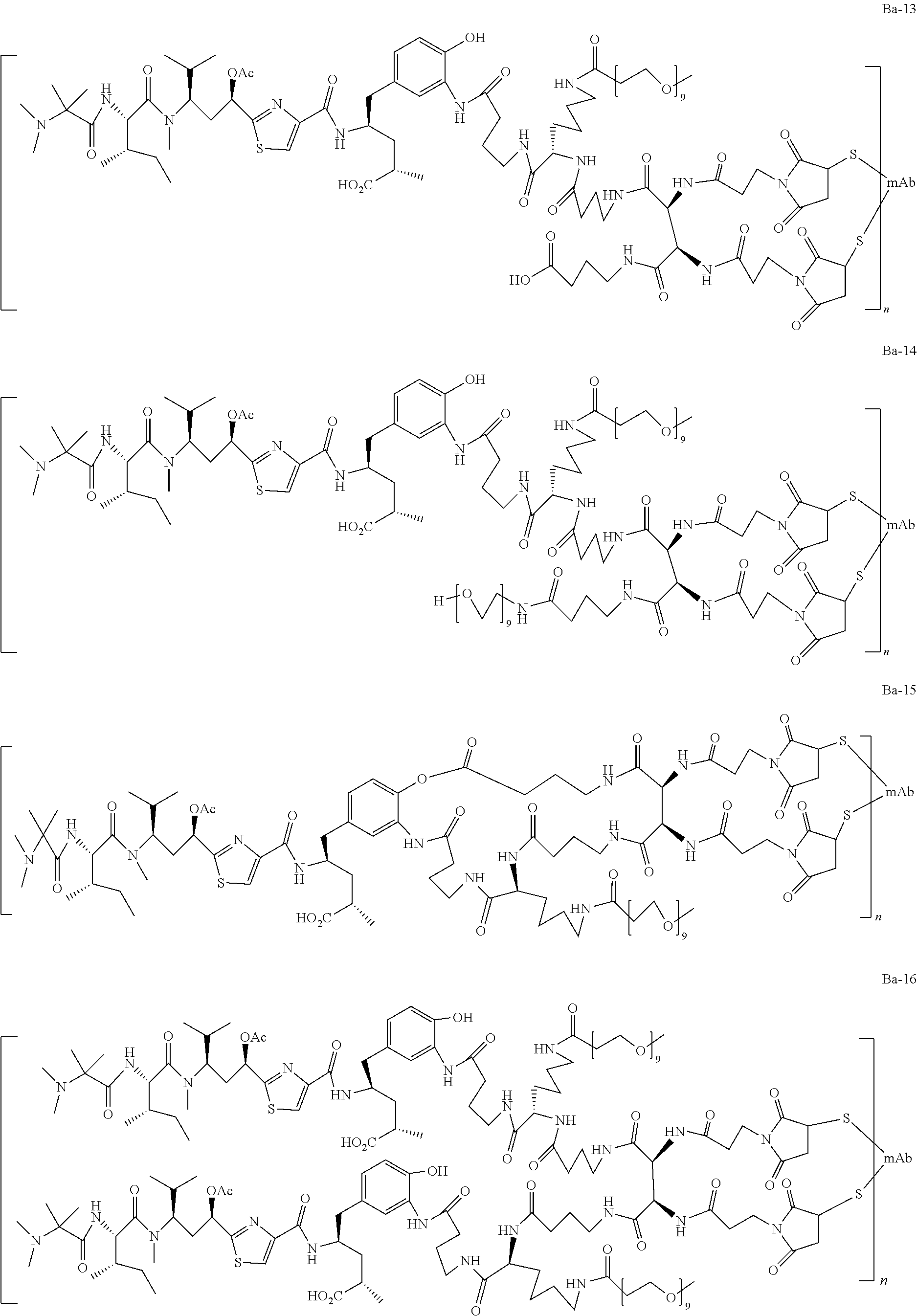

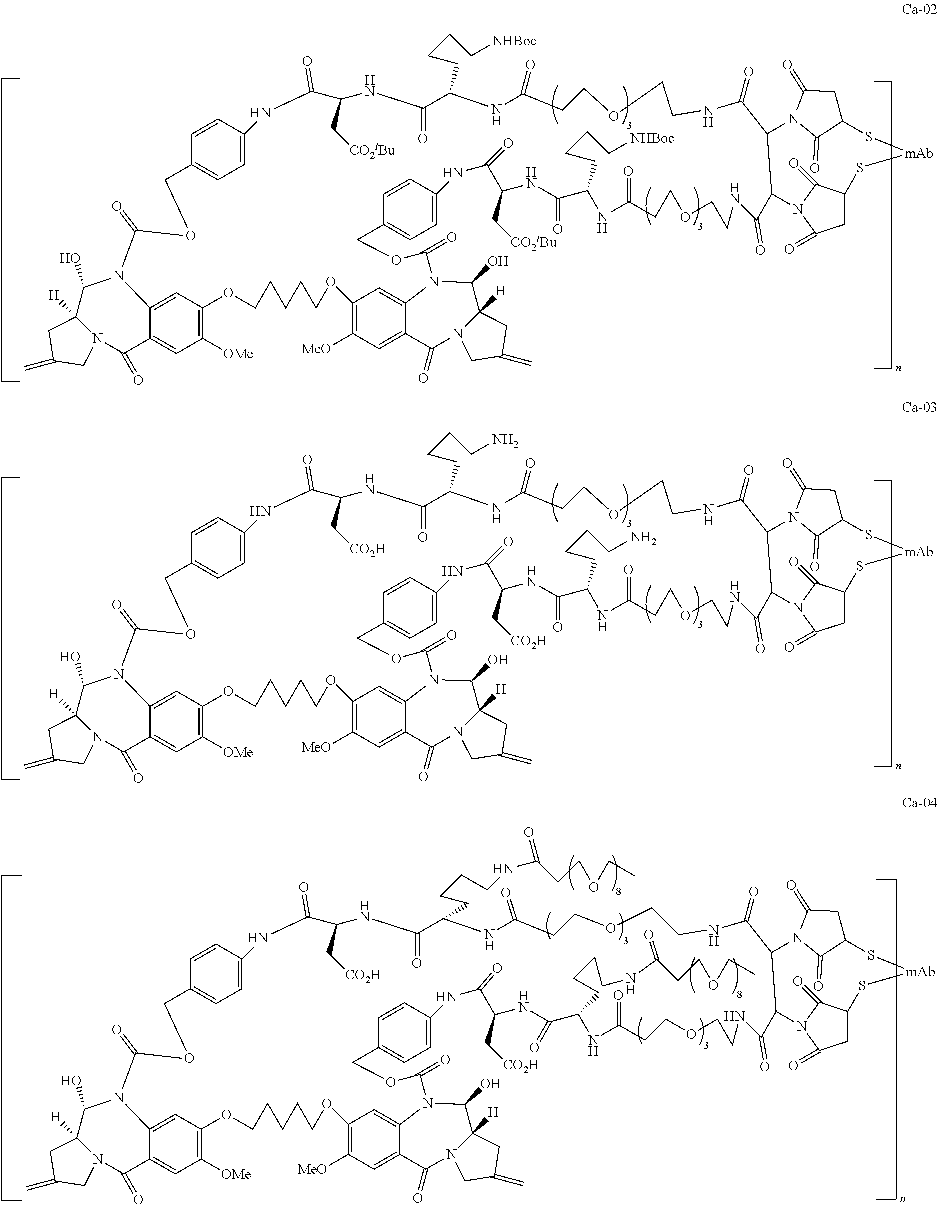

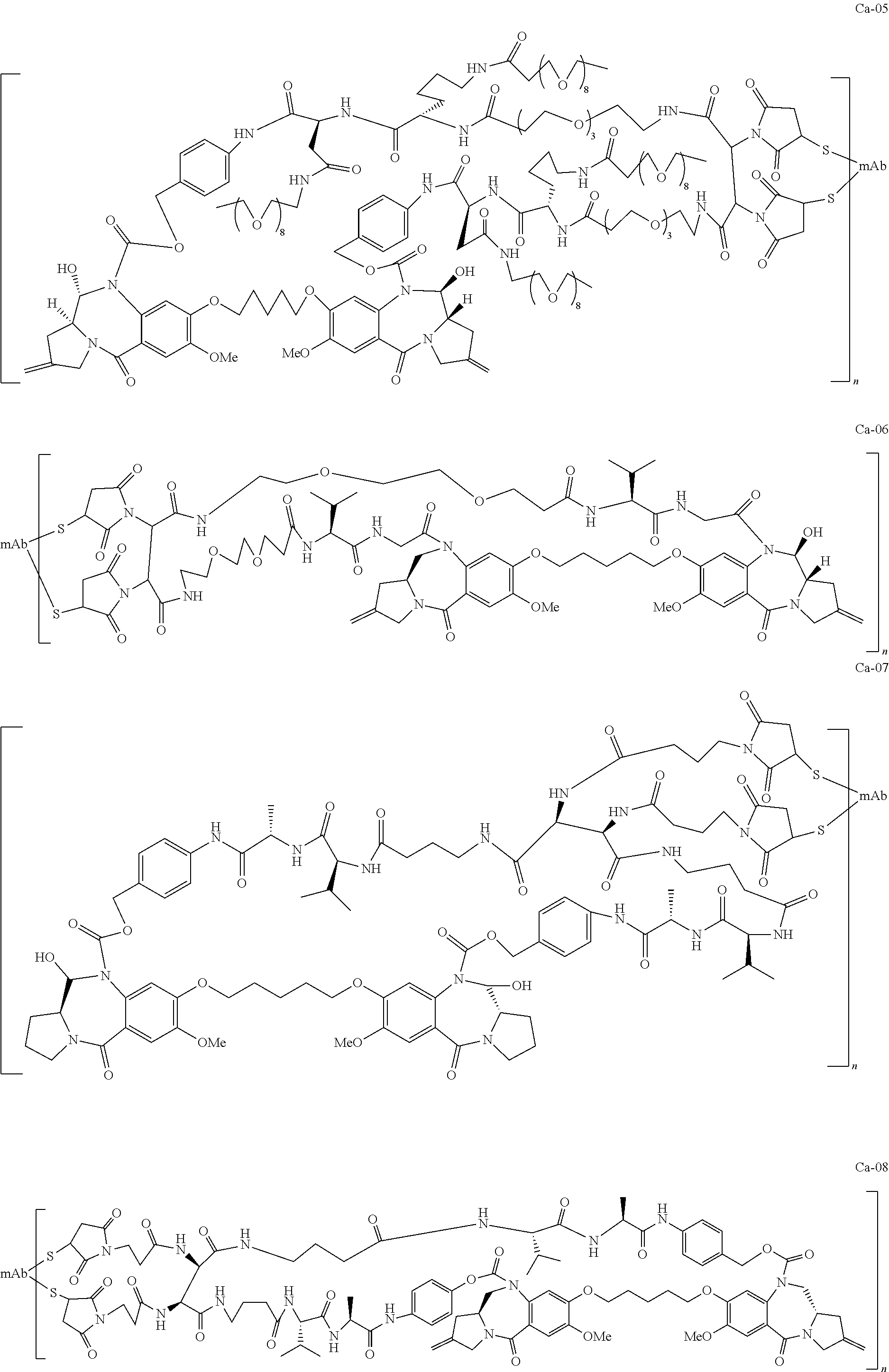

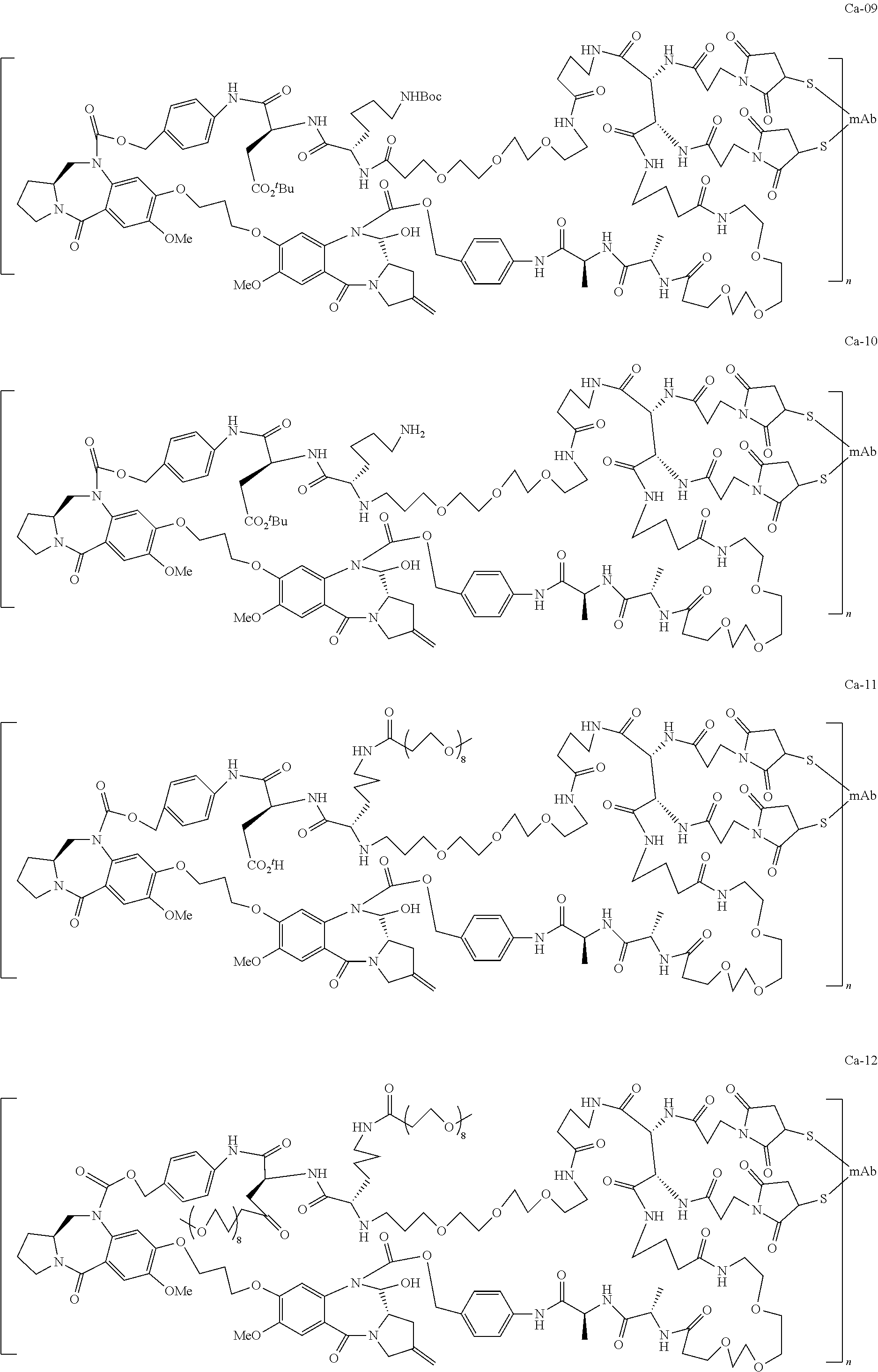

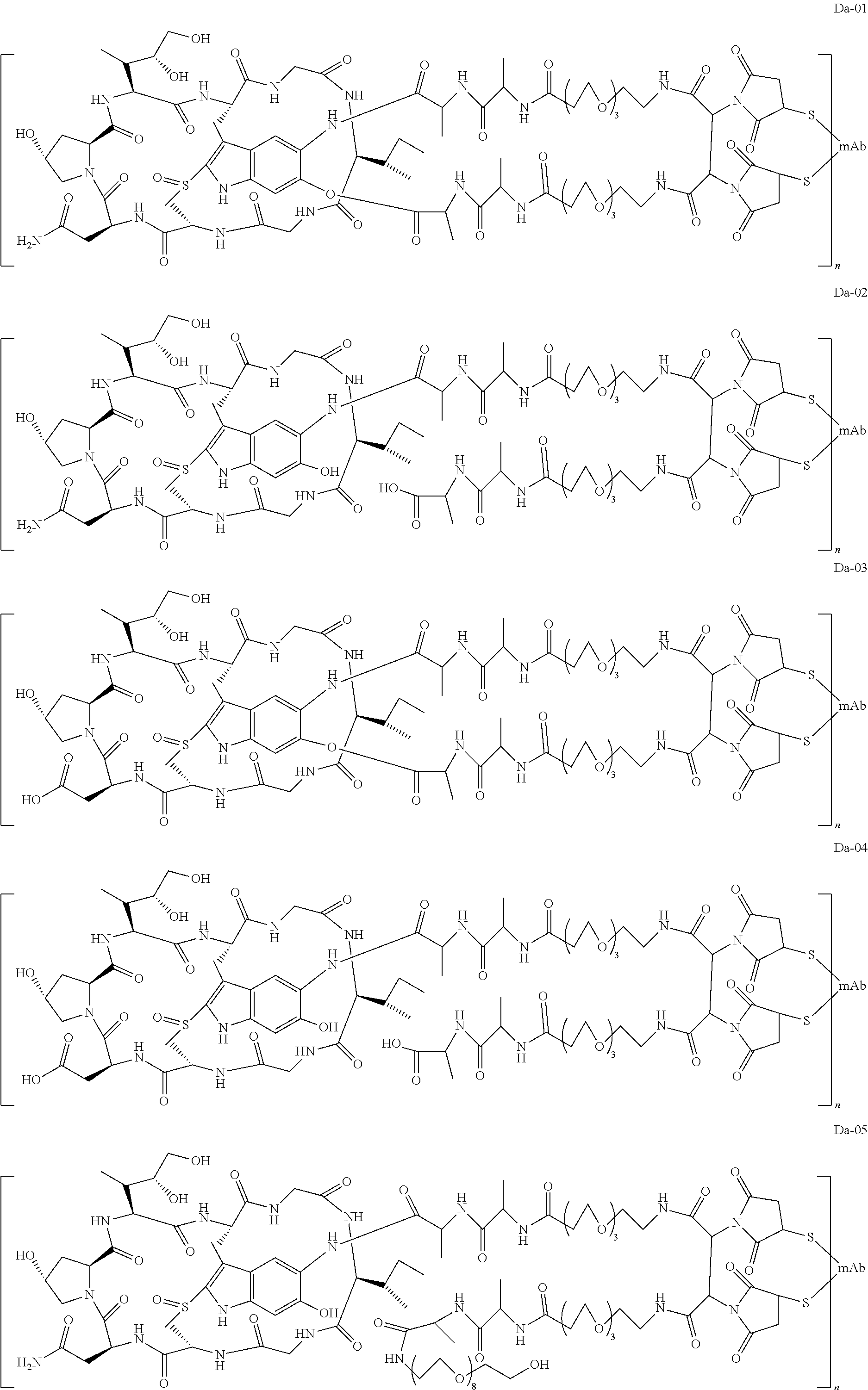

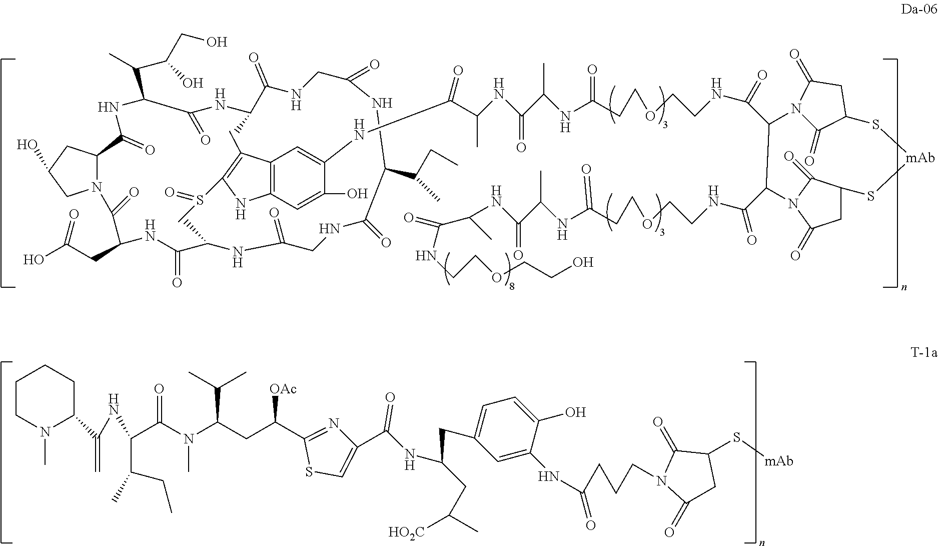

wherein n=2.0˜4.5

Example 340. In vitro cytotoxicity evaluation of conjugate Aa-01, Aa-02, Aa-03, Aa-04, Ba-01, Ba-02, Ba-03, Ba-04, Ba-05, Ba-06, Ba-07, Ba-08, Ba-09, Ba-10, Ba-11, Ba-12, Ba-13, Ba-14, Ba-15, Ba-16, Ca-02, Ca-03, Ca-04, Ca-05, Ca-06, Ca-07, Ca-08, Ca-09, Ca-10, Ca-11, Ca-12, Da-01, Da-02, Da-03, Da-04, Da-05 or Da-06 in comparison with T-DM1.

The cell line used in the cytotoxicity assays was NCI-N87, a human gastric carcinoma cell line; The cells were grown in RPMI-1640 with 100% FBS. To run the assay, the cells (180 μl, 6000 cells) were added to each well in a 96-well plate and incubated for 24 hours at 37° C. with 5% CO₂. Next, the cells were treated with test compounds (20 μl) at various concentrations in appropriate cell culture medium (total volume, 0.2 mL). The control wells contain cells and the medium but lack the test compounds. The plates were incubated for 120 hours at 37° C. with 50 CO₂. MTT (5 mg/ml) was then added to the wells (20 μl) and the plates were incubated for 1.5 hr at 37° C. The medium was carefully removed and DMSO (180 μl) was added afterward. After it was shaken for 15 min, the absorbance was measured at 490 nm and 570 nm with a reference filter of 620 nm. The inhibition % was calculated according to the following equation: inhibition %=[1−(assay-blank)/(control-blank)]×100.

The cytotoxicity results of IC₅₀ and IC₉₀:

| | DAR (drug | N87 cell (Ag+) | | N87 cell (Ag+) | |
|---|---|---|---|---|---|
| | ratio) | IC₅₀ (nM) | | IC₉₀ (nM) | |
| Conjugate Aa-01 | 4.0 | 0.97 | nM | 1.45 | nM |
| Conjugate Aa-02 | 3.7 | 0.36 | nM | 0.91 | nM |
| Conjugate Aa-03 | 3.8 | 0.67 | nM | 1.17 | nM |
| Conjugate Aa-04 | 4.1 | 0.34 | nM | 0.71 | nM |
| Conjugate Ba-01 | 3.9 | 0.34 | nM | 0.68 | nM |
| Conjugate Ba-02 | 3.6 | 0.78 | nM | 1.45 | nM |
| Conjugate Ba-03 | 3.9 | 0.14 | nM | 0.28 | nM |
| Conjugate Ba-04 | 3.8 | 0.42 | nM | 1.10 | nM |
| Conjugate Ba-05 | 3.8 | 0.83 | nM | 1.46 | nM |
| Conjugate Ba-06 | 3.8 | 0.72 | nM | 1.82 | nM |
| Conjugate Ba-07 | 3.7 | 0.93 | nM | 1.93 | nM |
| Conjugate Ba-08 | 3.2 | 0.22 | nM | 0.72 | nM |
| Conjugate Ba-09 | 3.6 | 0.45 | nM | 0.78 | nM |
| Conjugate Ba-10 | 3.9 | 0.19 | nM | 0.69 | nM |
| Conjugate Ba-11 | 3.8 | 0.13 | nM | 0.76 | nM |
| Conjugate Ba-12 | 3.8 | 0.22 | nM | 0.78 | nM |
| Conjugate Ba-13 | 3.6 | 0.29 | nM | 0.93 | nM |
| Conjugate Ba-14 | 3.8 | 0.28 | nM | 0.81 | nM |
| Conjugate Ba-15 | 3.8 | 0.83 | nM | 1.26 | nM |
| Conjugate Ba-16 | 3.8 | 0.22 | nM | 0.82 | nM |
| Conjugate Ca-02 | 3.7 | 0.93 | nM | 1.93 | nM |
| Conjugate Ca-03 | 4.1 | 0.07 | nM | 0.12 | nM |
| Conjugate Ca-04 | 3.8 | 0.039 | nM | 0.22 | nM |
| Conjugate Ca-05 | 4.0 | 0.10 | nM | 0.82 | nM |
| Conjugate Ca-06 | 3.8 | 1.13 | nM | 3.48 | nM |
| Conjugate Ca-07 | 3.8 | 0.021 | nM | 0.088 | nM |
| Conjugate Ca-08 | 3.8 | 61.36 | nM | 98.6 | nM |
| Conjugate Ca-09 | 2.1 | 0.51 | nM | 1.252 | nM |
| Conjugate Ca-10 | 3.9 | 0.13 | nM | 0.72 | nM |
| Conjugate Ca-11 | 3.8 | 0.43 | nM | 1.10 | nM |
| Conjugate Ca-12 | 3.6 | 0.71 | nM | 1.32 | nM |
| Conjugate Da-01 | 3.8 | 0.04 | nM | 0.087 | nM |
| Conjugate Da-02 | 3.8 | 0.038 | nM | 0.12 | nM |
| Conjugate Da-03 | 3.9 | 0.033 | nM | 0.072 | nM |
| Conjugate Da-04 | 3.8 | 0.047 | nM | 1.31 | nM |
| Conjugate Da-05 | 3.8 | 0.21 | nM | 0.82 | nM |
| Conjugate Da-06 | 3.9 | 0.34 | nM | 0.73 | nM |
| Conjugate T-1a | 3.8 | 0.25 | nM | 0.51 | nM |
| T-DM1 | 3.5 | 0.12 | nM | 0.26 | nM |

### Example 341. Antitumor Activity In Vivo (BALB/c Nude Mice Bearing NCI-N87 Xenograft Tumor)

The in vivo efficacy of conjugates Ba-12, Ba-14, Ba-16, Ca-03, Ca-04, Ca-05, Ca-06, Ca-07, Ca-10, Ca-11, Ca-12, along with T-DM1 were evaluated in a human gastric carcinoma N-87 cell line tumor xenograft models. Five-week-old female BALB/c Nude mice (104 animals) were inoculated subcutaneously in the area under the right shoulder with N-87 carcinoma cells (5×10⁶ cells/mouse) in 0.1 mL of serum-free medium. The tumors were grown for 8 days to an average size of 130 mm3. The animals were then randomly divided into 13 groups (8 animals per group). The first group of mice served as the control group and was treated with the phosphate-buffered saline (PBS) vehicle. 12 groups were treated with conjugates Ba-12, Ba-14, Ba-16, Ca-03, Ca-04, Ca-05, Ca-06, Ca-07, Ca-10, Ca-11, Ca-12, and T-DM1 respectively at dose of 3 mg/Kg administered intravenously. The remaining 2 groups were treated with conjugate C-3a and D-1a respectively at dose of 1 mg/Kg administered intravenously. Three dimensions of the tumor were measured every 4 days and the tumor volumes were calculated using the formula tumor volume=½ (length width x height). The weight of the animals was also measured at the same time. A mouse was sacrificed when any one of the following criteria was met: (1) loss of body weight of more than 20% from pretreatment weight, (2) tumor volume larger than 2000 mm³, (3) too sick to reach food and water, or (4) skin necrosis. A mouse was considered to be tumor-free if no tumor was palpable.

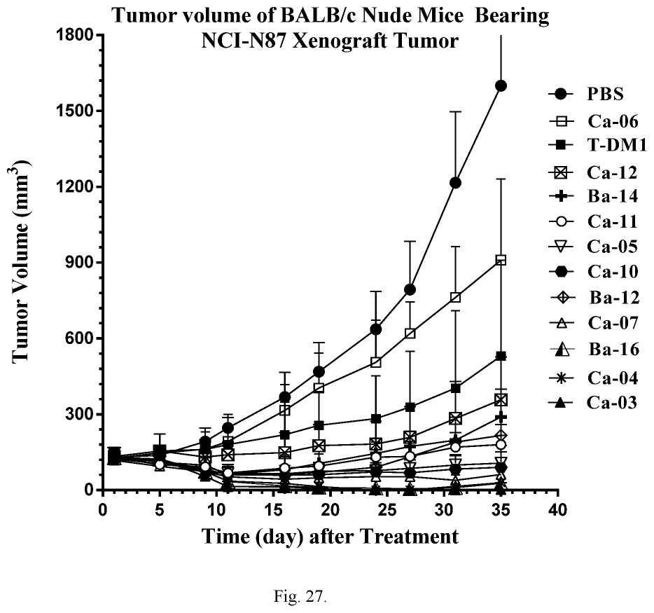

The results were plotted in FIG. 27. All the 12 conjugates did not cause the animal body weight loss. And the animals at control group were sacrificed at day 35 due to the tumor volume larger than 2000 mm³ and some of them were too sick. Here 12 conjugates except Ca-06 tested demonstrated better anti-tumor activity than T-DM1. All 6/6 animals at the groups of compounds Ca-04 and Ca-3 had completely no tumor measurable at day 14 till day 30. In contrast T-DM1 at dose of 3 mg/Kg was not able to eliminate the tumors.

1-47. (canceled)

48. A conjugate compound having a stereoisomeric structure of 2,3-diaminosuccinyl group represented by Formula (Ia), (Ib), (Ic), (IIa), (IIb), (IIc), (IIIa), (IIIb), (IIIc), (IVa), (IVb) or (IVc) below 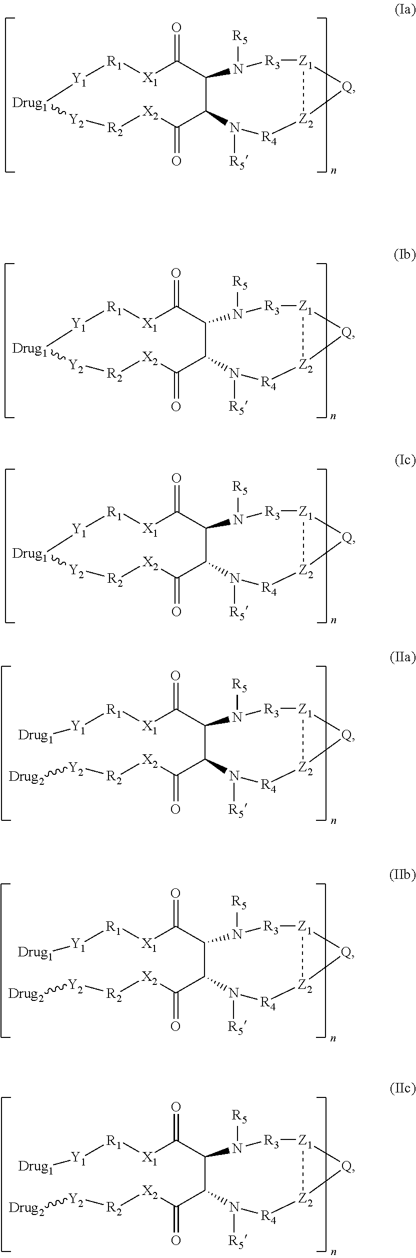 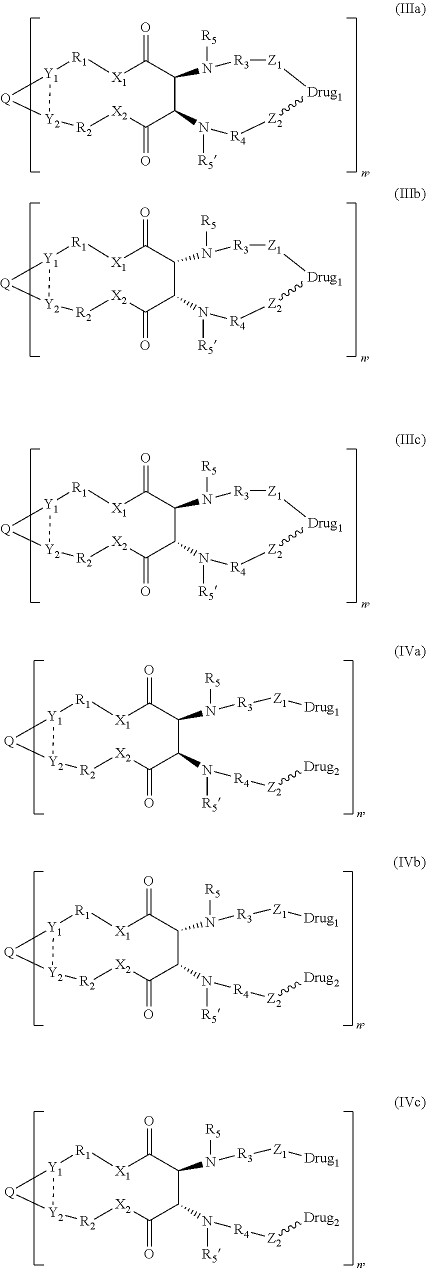
wherein
“ [symbol] ” represents a single bond;
“ [symbol] ” is optionally either a single bond, or absent;
“-----” optionally either a single bond, or a double bond, or can optionally be absent;
n is 1 to 30 independently;
Q is a cell-binding agent that links to R₃ and R₄, of a molecule that binds to, complexes with, or reacts with a moiety of a cell population sought to be therapeutically or otherwise biologically modified; the cell-binding agent is an immunotherapeutic protein, an antibody, a single chain antibody; an antibody fragment that binds to the target cell; a monoclonal antibody; a single chain monoclonal antibody; or a monoclonal antibody fragment that binds the target cell; a chimeric antibody; a chimeric antibody fragment that binds to the target cell; a domain antibody; a domain antibody fragment that binds to the target cell; adnectins that mimic antibodies; DARPins; a lymphokine; a hormone; a vitamin; a growth factor; a colony stimulating factor; or a nutrient-transport molecule (a transferrin); a binding peptide having over four aminoacids, or protein, or antibody, or small cell-binding molecule or ligand attached on albumin, polymers, dendrimers, liposomes, nanoparticles, vesicles, or (viral) capsids;
Drug₁ and Drug₂ are each a cytotoxic agent that is a therapeutic drug, or an immunotherapeutic molecule, or a function molecule for enhancement of binding or stabilization of the cell-binding agent, or a cell-surface receptor binding ligand, or for inhibition of cell proliferation, or for monitoring, detection or study of a cell-binding molecule action, an analog, or prodrug, or a pharmaceutically acceptable salt, hydrate, or hydrated salt, or a crystalline structure, or an optical isomer, racemate, diastereomer or enantiomer, of immunotherapeutic compound, a chemotherapeutic compound, an antibody (probody) or an antibody (probody) fragment, or siRNA or DNA molecule, or a cell surface binding ligand;
X₁ and X₂ are the same or different, and independently selected from the group consisting of NH; NHNH; N(R₁); N(R₁)N(R₂); O; S; S—S, O—NH, O—N(R₁), CH₂—NH, CH₂—N(R₁), CH═NH, CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NH)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); C₁-C₆ alkyl; C₂-C₈ alkenyl, heteroalkyl, alkylcycloalkyl, or heterocycloalkyl; and C₃-C₈ aryl, Ar-alkyl, heterocyclic, carbocyclic, cycloalkyl, heteroalkylcycloalkyl, alkylcarbonyl, or heteroaryl;
Y₁, Y₂, Z₁ and Z₂ are, the same or different, and independently a function group that link to a cell-binding molecule Q, or Drug, or Drug₂, in a form of a disulfide, ether, ester, thioether, thioester, peptide, hydrazone, carbamate, carbonate, amine (secondary, tertiary, or quarter), imine, cycloheteroalkyane, heteroaromatic, alkyloxime or amide bond; Y₁, Y₂, Z₁ and Z₂ independently have one of the following structures: C(O)CH, C(O)C, C(O)CH₂, ArCH₂, C(O), NH, NHNH, N(R₁), N(R₁)N(R₂), O, S, S—S, O—NH, O—N(R₁), CH₂—NH. CH₂—N(R₁), CH═NH. CH═N(R₁), S(O), S(O₂), P(O)(OH), S(O)NH, S(O₂)NH, P(O)(OH)NH, NHS(O)NH, NHS(O₂)NH, NHP(O)(OH)NH, N(R₁)S(O)N(R₂), N(R₁)S(O₂)N(R₂), N(R₁)P(O)(OH)N(R₂), OS(O)NH, OS(O₂)NH, OP(O)(OH)NH, C(O), C(NH), C(NR₁), C(O)NH, C(NH)NH, C(NR₁)NH, OC(O)NH, OC(NH)NH; OC(NR₁)NH, NHC(O)NH; NHC(NH)NH; NHC(NR₁)NH, C(O)NH, C(NR₁)NH, OC(O)N(R₁), OC(NH)N(R₁), OC(NR₁)N(R₁), NHC(O)N(R₁), NHC(NH)N(R₁), NHC(NR₁)N(R₁), N(R₁)C(O)N(R₁), N(R₁)C(NH)N(R₁), N(R₁)C(NR₁)N(R₁); or C₁-C₈ alkyl, C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl;
R₁, R₂, R₃, and R₄ are, the same or different, independently selected from the group consisting of O, NH, S, NHNH, N(R₅), N(R₃)N(R₃′), polyethyleneoxy unit of formula (OCH₂CH₂)ₚOR₅, or (OCH₂CH—(CH₃))ₚOR₅, or NH(CH₂CH₂O)ₚR₅, or NH(CH₂CH(CH₃)O)ₚR₅, or N[(CH₂CH₂O)ₚR₅]—[(CH₂CH₂O)_{p′}R_{5′}], or (OCH₂CH₂)ₚCOOR₅, or CH₂CH₂(OCH₂CH₂)ₚCOOR₅, wherein p and p′ are independently an integer selected from 0 to about 1000, or combination thereof; C₁-C₈ alkyl; C₂-C₈ heteroalkyl, alkylcycloalkyl, heterocycloalkyl, esters, ether, or amide; C₃-C₈ aryl, Ar-alkyl, heterocyclic, carbocyclic, cycloalkyl, heteroalkylcycloalkyl, alkylcarbonyl, or heteroaryl; or 1-24 amino acids; wherein R₅ and R_{5′} are independently H; C₁-C₈ alkyl; C₂-C₈ heteroalkyl, alkylcycloalkyl, or heterocycloalkyl; C₃-C₈ aryl, Ar-alkyl, heterocyclic; C₂-C₈ carbon atoms of esters, ether, or amide; or 1-24 amino acids;
or R₁, R₂, R₃, and R₄ may optionally be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1-8 natural or unnatural amino acid unites; the natural aminoacid being selected from aspartic acid, glutamic acid, arginine, histidine, lysine, serine, threonine, asparagine, glutamine, cysteine, selenocysteine, tyrosine, phenylalanine, glycine, proline, tryptophan, and alanine;
or R₁, R₂, R₃, and R₄ may independently contain one or more of the following hydrophilic structures: 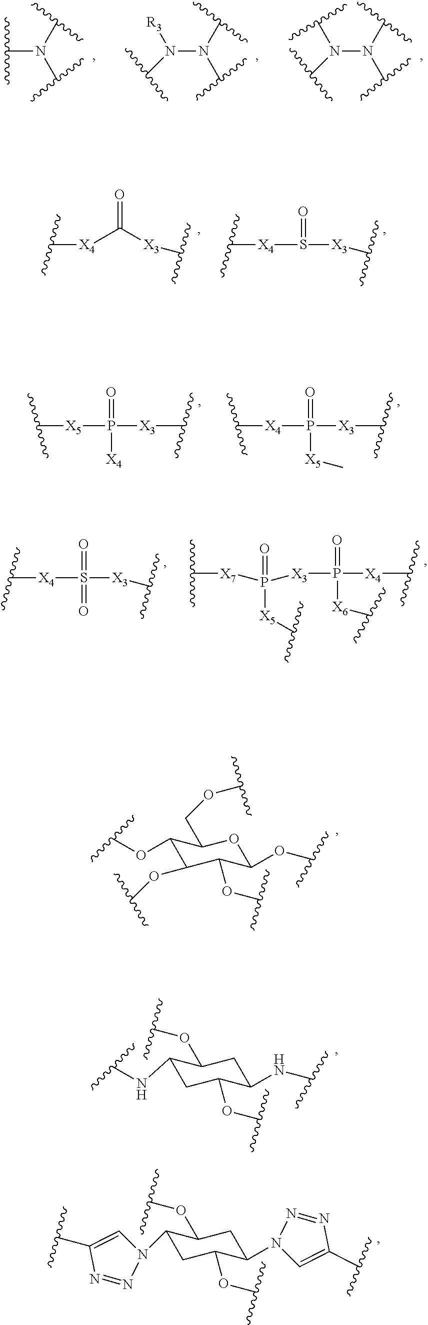 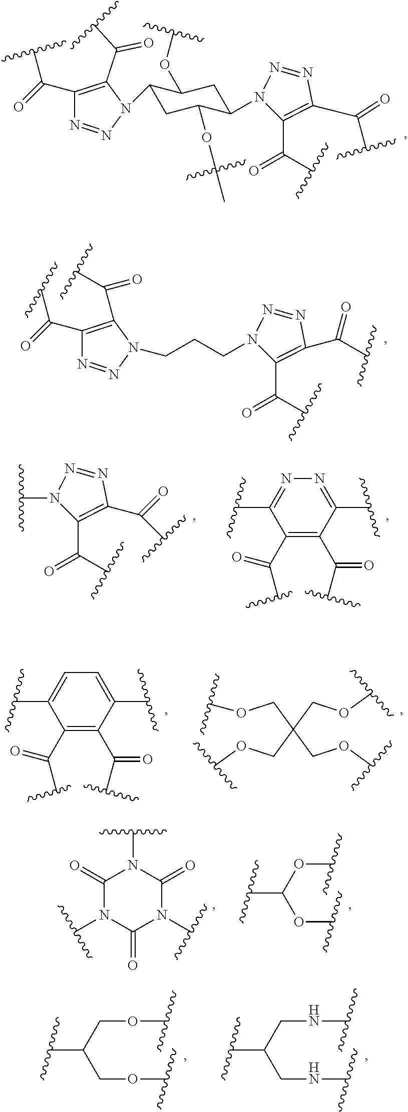 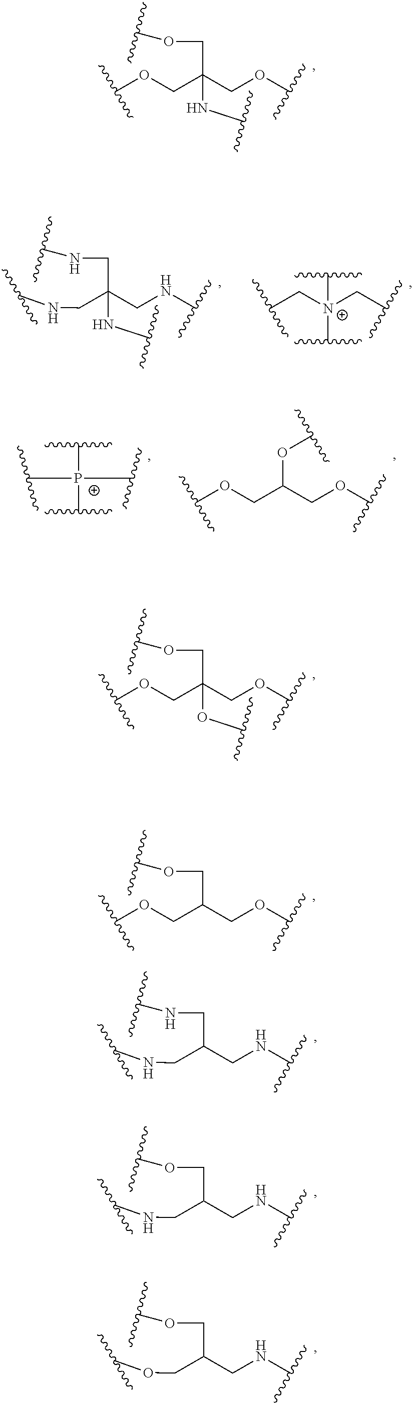
wherein [symbol] is the site of linkage; X₃, X₄, X₅, X₆, and X₇, are independently NH; NHNH; N(R₅); N(R₅)N(R₅); O; S; C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, heteroaryl; or 1-8 amino acid; wherein R₅ and R_{5′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, heteroalkylcycloalkyl, alkylcarbonyl, or heteroaryl; C₁—C ester, ether, or amide; or polyethyleneoxy having formula (OCH₂CH₂)ₚ or (OCH₂CH(CH₃))ₚ, wherein p is an integer from 0 to about 5000, or combination above thereof;
or R₁, R₂, R₃, R₄, Y₁, Y₂, Z₁, and Z₂ are independently contain a self-immolative or a non-self-immolative component, peptidic units, a hydrazone bond, a disulfide, an ester, an oxime, an amide, or a thioether bond; the self-immolative unit includes, aromatic compounds that are electronically similar to the para-aminobenzylcarbamoyl (PAB) groups such as 2-aminoimidazol-5-methanol derivatives, heterocyclic PAB analogs, beta-glucuronide, and ortho or para-aminobenzylacetals;
the self-immolative linker component has one of the following structures: 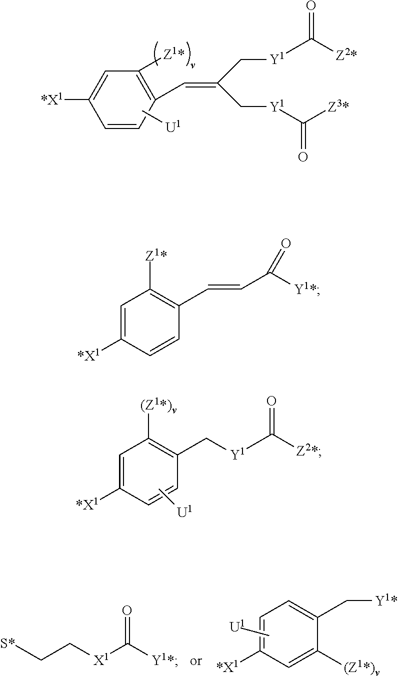
wherein the (*) atom is the point of attachment of additional spacer or releasable linker units, or the cytotoxic agent, and/or the binding molecule (CBA); X¹, Y¹, Z² and Z³ are independently NH, O, or S; Z¹ is independently H, NHR₅, OR₁, SR₅, COX₁R₅, wherein X₁ and R₅ are defined above; v is 0 or 1; U¹ is independently H, OH, C₁-C₆ alkyl, (OCH₂CH₂)ₙ, F, Cl, Br, I, OR₅, SR₅, NR₅R_{5′}, N═NR₅, N═R₅, NR₅R_{5′}, NO₂, SOR₅R_{5′}, SO₂R₅, SO₃R₅, OSO₃R₅, PR₅R_{5′}, POR₅R_{5′}, PO₂R₅R_{5′}, OPO(OR₅)(OR_{5′}), or OCH₂PO(OR₅(OR_{5′}), wherein R₅ and R_{5′} are independently selected from H, C₁-C₈ alkyl; C₂-C₈ alkenyl, alkynyl, heteroalkyl, or amino acid; C₃-C₈ aryl, heterocyclic, carbocyclic, cycloalkyl, heterocycloalkyl, heteroaralkyl, alkylcarbonyl, or glycoside; or pharmaceutical cation salts;
the non-self-immolative linker component is one of the following structures: 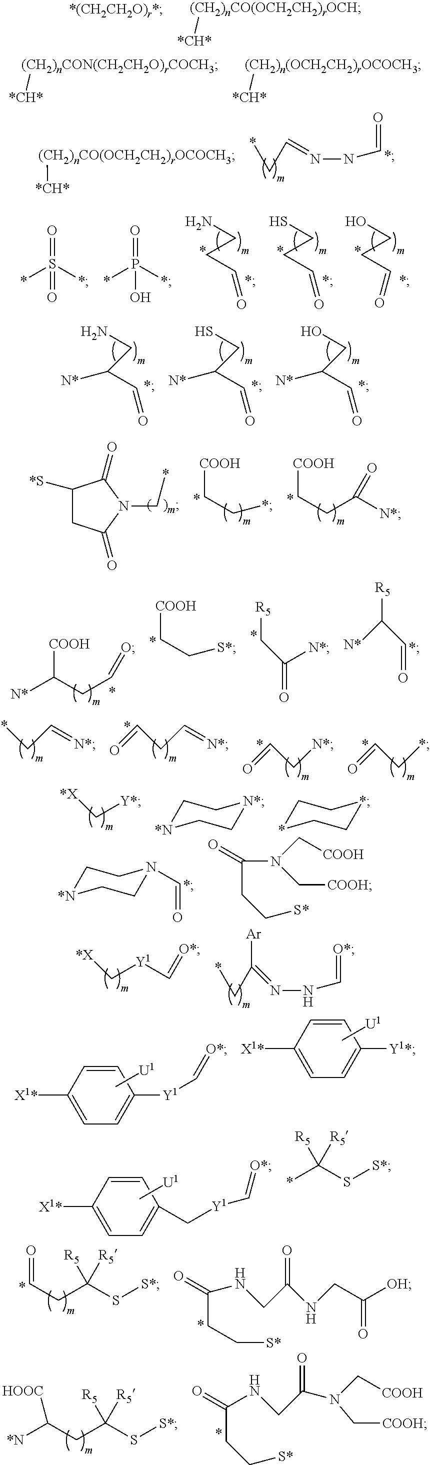 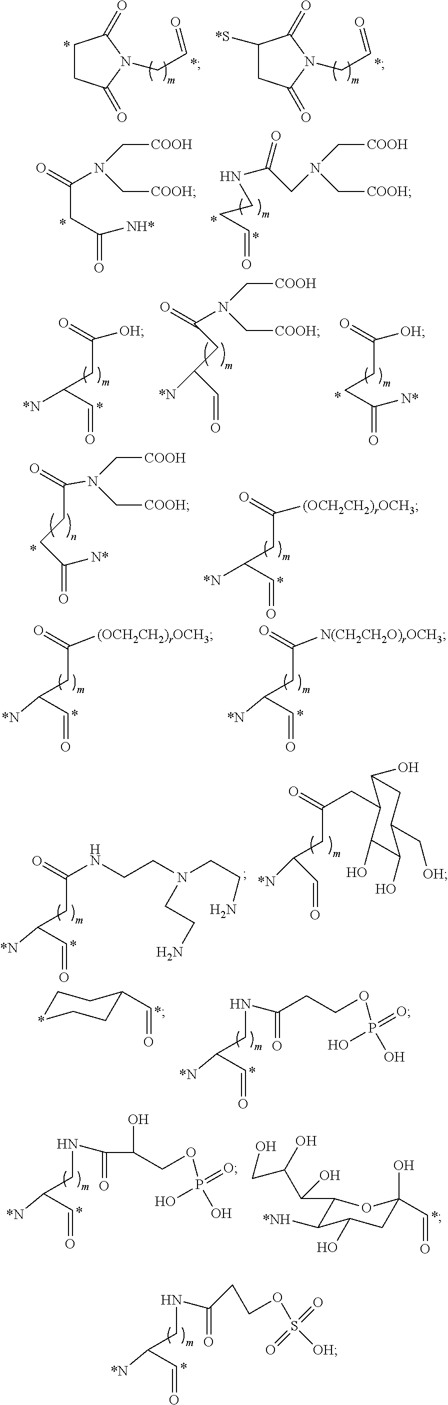 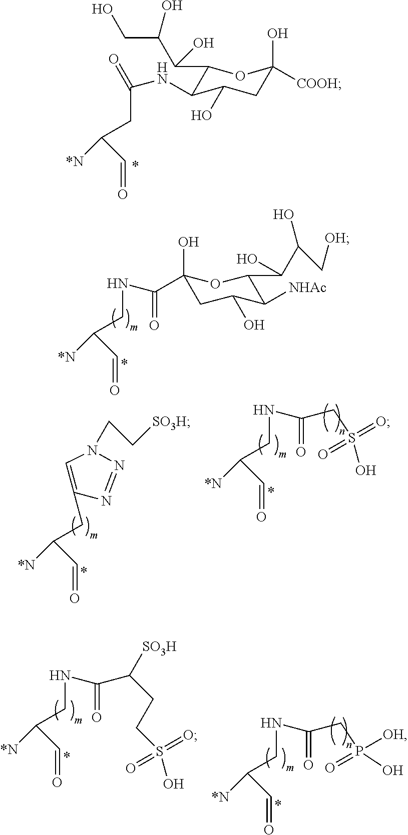
wherein the (*) atom is a point of attachment of additional spacer or releasable linkers, the cytotoxic agent, and/or the binding molecule; X¹, Y¹, U¹, R₅, R_{5′} are defined as above; r is 0-100; m and n are 0-20 independently;
or R₁, R₂, R₃, and R₄ may independently contain a releasable linker component which includes at least one bond that can be broken under physiological conditions, and is a pH-labile, acid-labile, base-labile, oxidatively labile, metabolically labile, biochemically labile or enzyme-labile bond having one of the following structures:
—(CR₅R₆)ₘ(Aa)ᵣ(CR₇R₈)ₙ(OCH₂CH₂)ᵣ, —(CR₅R₆)ₘ(CR₇R₈)ₙ(Aa)ᵣ(OCH₂CH₂)ₜ—, - (Aa)ᵣ (CR₅R₆)ₘ(CR₇R₈)ₙ(OCH₂CH₂)ᵣ, —(CR₅R₆)ₘ(CR₇R₈)ₙ(OCH₂CH₂)ᵣ(Aa)ₜ, —(CR₅R₆)ₘ—(CR₇═CR₈)(CR₉R₁₀)ₙ(Aa)ₜ(OCH₂CH₂)ᵣ, —(CR₅R₆)ₘ(NR₁₁CO)(Aa)ₜ(CR₉R₁₀)ₙ₋(OCH₂CH₂)ᵣ, —(CR₅R₆)ₘ(Aa)ₜ(NR₁₁CO)(CR₉R₁₀)ₙ(OCH₂CH₂)ᵣ, —(CR₅R₆)ₘ(OCO)(Aa)ₜ(CR₉R₁₀)ₙ₋(OCH₂CH₂)ᵣ, —(CR₅R₆)ₘ(OCNR₇)(Aa)ₜ(CR₉R₁₀)ₙ(OCH₂CH₂)ᵣ, —(CR₅R₆)ₘ(CO)(Aa)ₜ-(CR₉R₁₀)ₙ(OCH₂CH₂)ᵣ, —(CR₅R₆)ₘ(NR₁₁CO)(Aa)ₜ(CR₉R₁₀)ᵣ(OCH₂CH₂)ᵣ, —(CR₅R₆)ₘ—(OCO)(Aa)ₜ(CR₉R₁₀)ₙ₋(OCH₂CH₂)ᵣ, —(CR₅R₆)ₘ(OCNR₇)(Aa)ₜ(CR₉R₁₀)ₙ(OCH₂CH₂)ᵣ, —(CR₅R₆)ₘ(CO)(Aa)ₜ(CR₉R₁₀)ₙ—(OCH₂CH₂)ᵣ, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CR₅R₆)ₙ-oxazolyl-CO(Aa)ₜ(CR₇R₈)ₙ—, —(CR₅R₆)ₘ-thiazolyl-CO(Aa)ₜ(CCR₇R₈)ₙ—, —(CR₅R₆)ₜ₋thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ₋(CR₇R₈)ₙ—, —(CR₅R₆)ₜpiperazino-CO(Aa)ₜ(CR₇R₈)ₙ—, —(CR₅R₆)ᵣ—N-methylpiperazin-CO(Aa)ₜ₋(CR₇R₈)ₙ—, —(CR₅R₆)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazolyl(Aa)ᵣ, —(CR₅R₆)ₘ-morpholino-(Aa)ᵣ, —(CR₅R₆)ₘ-piperazino-(Aa)ᵣ, —(CR₅R₆)ₘ—N-methylpiperazino-(Aa)ᵣ, —K(CR₅R₆)ₘ(Aa)ᵣ(CR₇R₈)ₙ(OCH₂CH₂)ᵣ, —K(CR₅R₆)ₘ(CR₇R₈)ₙ(Aa)ᵣ(OCH₂CH₂)ₜ—, —K(Aa)ᵣ (CR₅R₆)ₘ(CR₇R₈)ₙ(OCH₂CH₂)ₜ, —K(CR₅R₆)ₘ(CR₇R₈)ₙ(OCH₂CH₂)ᵣ(Aa)ᵣ, —K(CR₅R₆)ₘ—(CR₇═CR₈)(CR₉R₁₀)ₙ(Aa)ₜ(OCH₂CH₂)ᵣ, —K(CR₅R₆)ₘ(NR₁₁CO)(Aa)ₜ(CR₉R₁₀)ₙ(OCH₂CH₂)ᵣ, —K(CR₅R₆)ₘ(Aa)ₜ(NR₁₁CO)(CR₉R₁₀)ₙ(OCH₂CH₂)ᵣ, —K(CR₅R₆)ₘ(OCO)(Aa)ₜ(CR₉R₁₀)ₙ₋(OCH₂CH₂)ᵣ, —K(CR₅R₆)ₘ(OCNR₇)(Aa)ₜ(CR₉R₁₀)ₙ(OCH₂CH₂)ᵣ, —K(CR₅R₆)ₘ(CO)(Aa)ₜ-(CR₉R₁₀)ₙ(OCH₂CH₂)ᵣ, —K(CR₅R₆)ₘ(NR₁₁CO)(Aa)ₜ(CR₉R₁₀)ₙ(OCH₂CH₂)ᵣ, —K(CR₅R₆)ₘ—(OCO)(Aa)ₜ(CR₉R₁₀)ₙ(OCH₂CH₂)ᵣ, —K(CR₅R₆)ₘ(OCNR₇)(Aa)ₜ(CR₉R₁₀)ₙ(OCH₂CH₂)ᵣ, —K—(CR₅R₆)ₘ(CO)(Aa)ₜ(CR₉R₁₀)ₙ(OCH₂CH₂)ᵣ, —K(CR₅R₆)ₘ-phenyl-CO(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)ₜimidazolyl-CO—(CR₇R₈)ₙ—, —K(CR₅R₆)ₜmorpholino-CO(Aa)ₜ(CR₇R₈)ₙ—, —K(CR₅R₆)ₜpiperazino-CO(Aa)ₜ(CR₇R₈)ₙ—, —K(CR₅R₆)ₜ—N-methylpiperazinCO(Aa)ₜ(CR₇R₈)ₙ—, —K(CR₅R₆)ₘ(Aa)ₜphenyl, —K—(CR₅R₆)ₘ-(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘN-methylpiperazino(Aa)ᵣ;
wherein Aa, m, and n are described above; t and r are 0-100 independently; R₃, R₄, R₅, R₆, R₇, and R₈ are independently chosen from H; halide; C₁-C₈ alkyl; C₂-C₈ aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—NH—, O, S, Se, B, Het (heterocyclic or heteroaromatic ring having C₃-C₈), or peptides containing 1-20 amino acids;
or R₁, R₂, R₃, and R₄, are independently linear alkyl having from 1-18 carbon atoms, or polyethyleneoxy unit having formula (OCH₂CH₂)ₚ, p=1-5000, or a peptide containing 1-20 units of aminoacids (L or D form), or combination above;
or Y₁, Y₂, R₁, R₂, R₃, R₄, Z₁ or Z₂ may independently be composed of one or more following components: 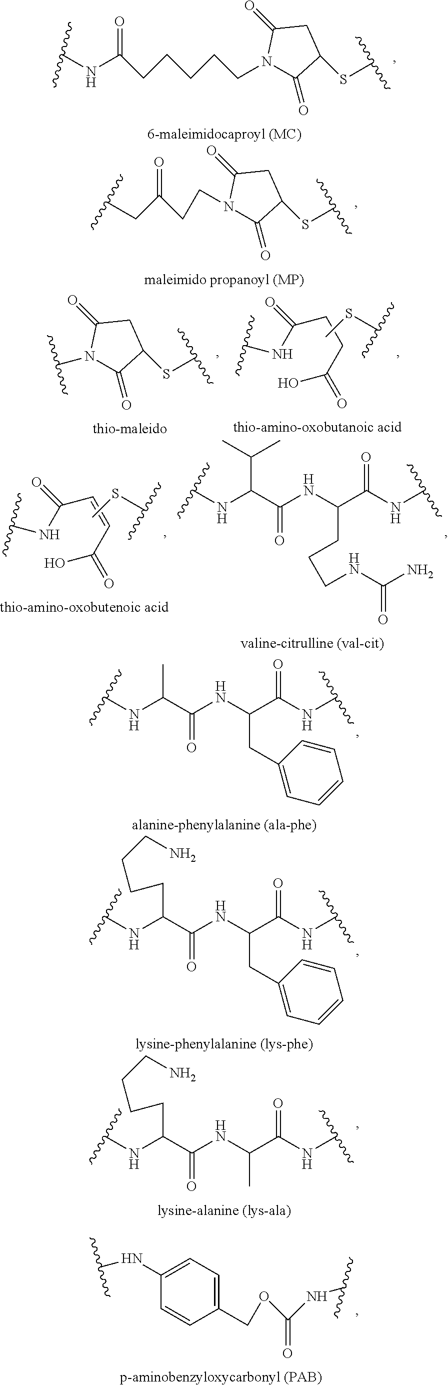 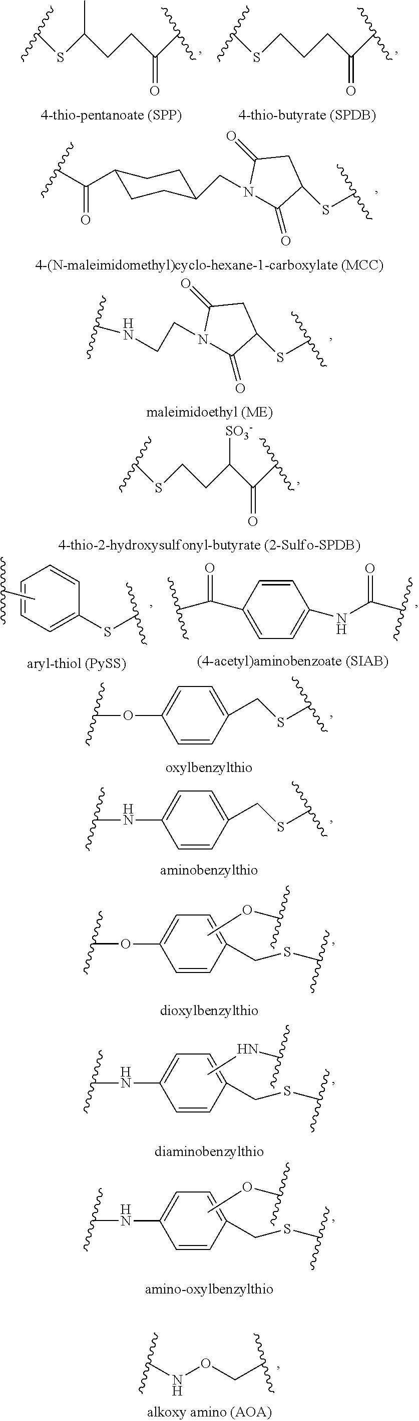 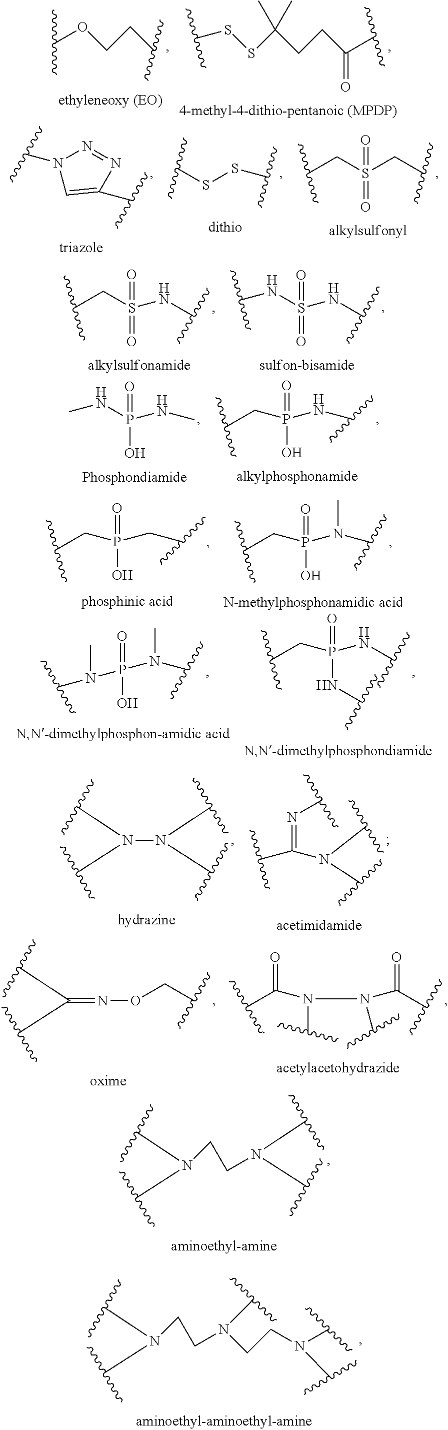
and L- or D-, natural or unnatural peptides containing 1-20 amino acids; wherein a connecting bond in the middle of atoms means that it can connect either neighbor carbon atom bonds; wavy line is the site wherein another bond can be connected to;
or Y₁, Y₂, R₁, R₂, R₃, R₄, Z₁ or Z₂ can be independently absent, provided that Y₁, Y₂, R₁, R₂, R₃, R₄, Z₁ and Z₂ may not be absent at the same time.

49. The conjugate compound according to claim 48, which is represented by Formula (I-01), (I-02), (I-03), (I-04), (I-05), (I-06), (I-07), (I-08), (I-09), (I-10), (I-11), (I-12), (I-13), (I-14), (I-15), (1-16), (I-17), (I-18), (I-19), (I-20), (I-21), (I-22), (I-23), (II-01), (II-02), (II-03), (II-04), (II-05), (II-06), (II-07), (II-08), (II-09), (II-10), (II-11), (II-12), (II-13), (II-14), (II-15), (II-16), (II-17), (II-18), (III-01), (III-02), (III-03), (III-04), (III-05), (III-06), (III-07), (III-08), (III-09), (III-10), (III-11), (III-12), (III-13), (III-14), (III-15), (III-16), (III-17), (III-18), (III-19), (III-20), (IV-01), (IV-02), (IV-03), (IV-04), (IV-05), (IV-06), (IV-07), (IV-08), (IV-09), (IV-10), (IV-11), (IV-12), (IV-13), (IV-14), (IV-15), (IV-16), (IV-17), (IV-18), (IV-19), or (IV-20) below: 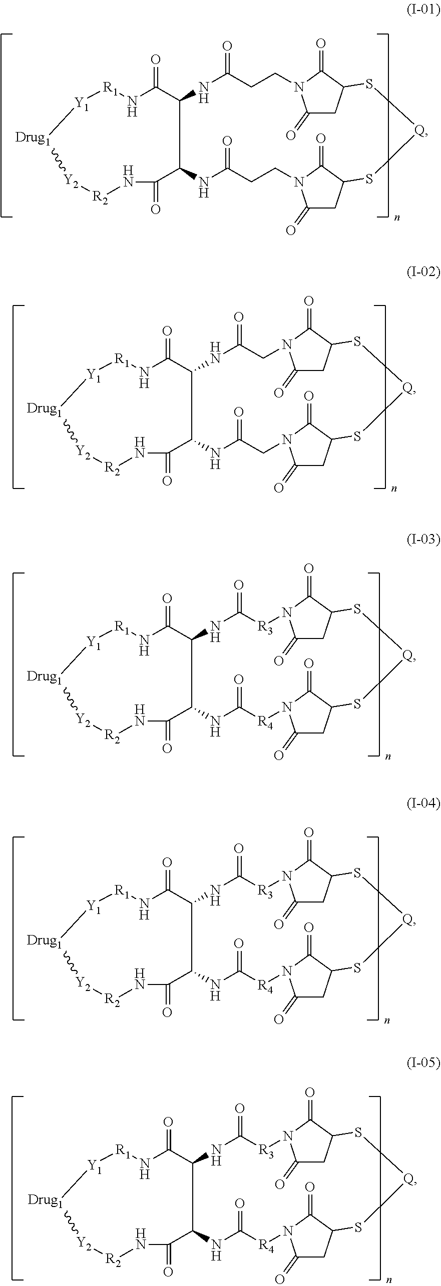 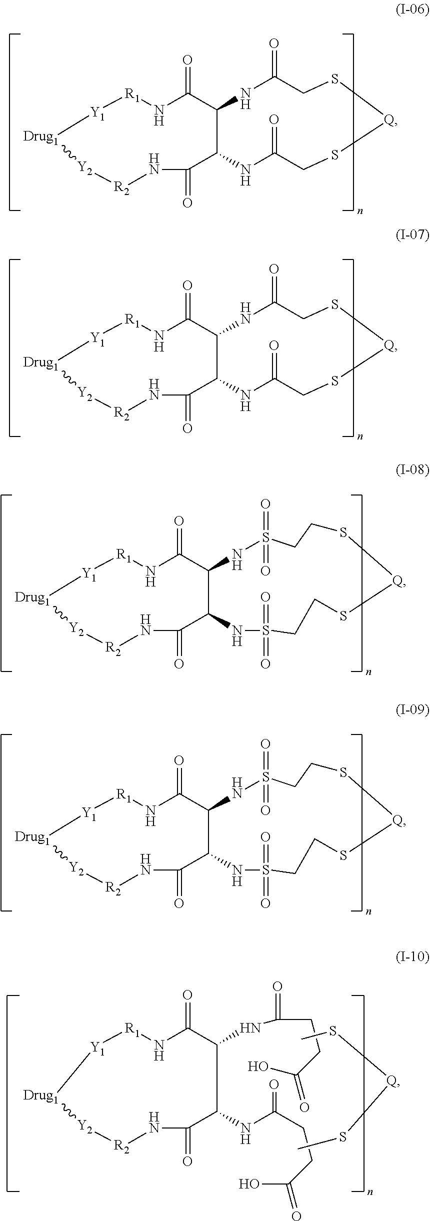 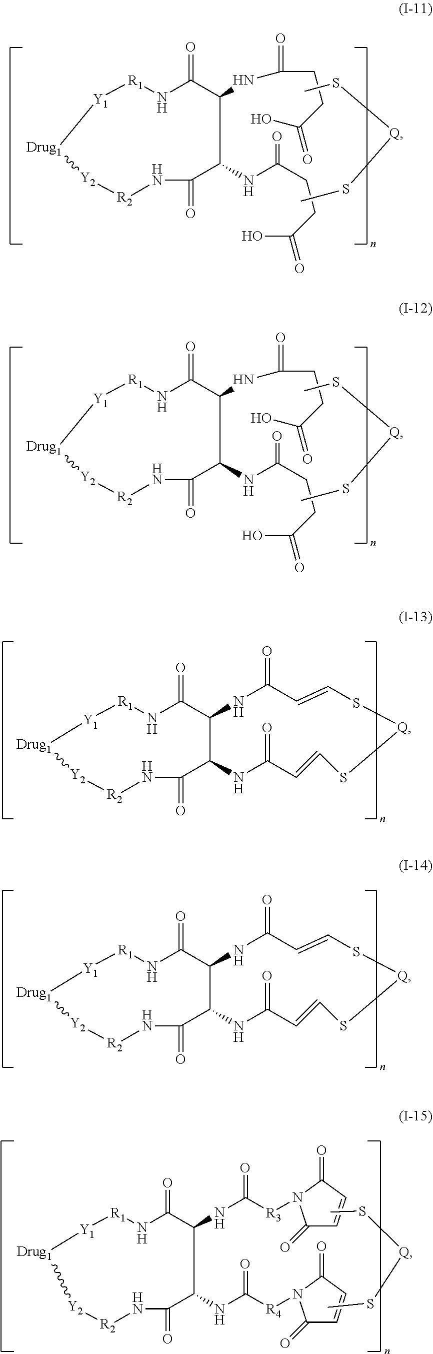 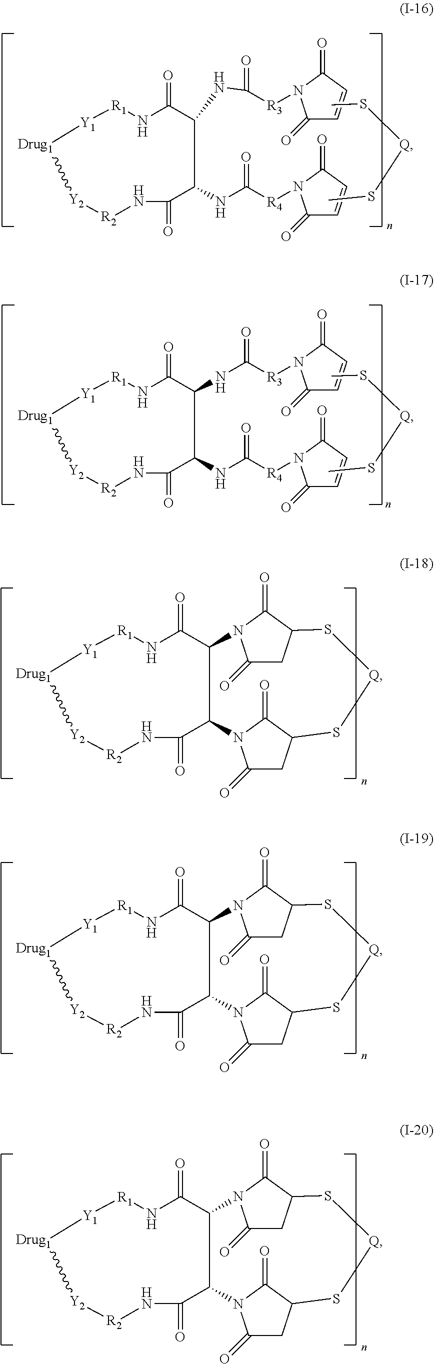 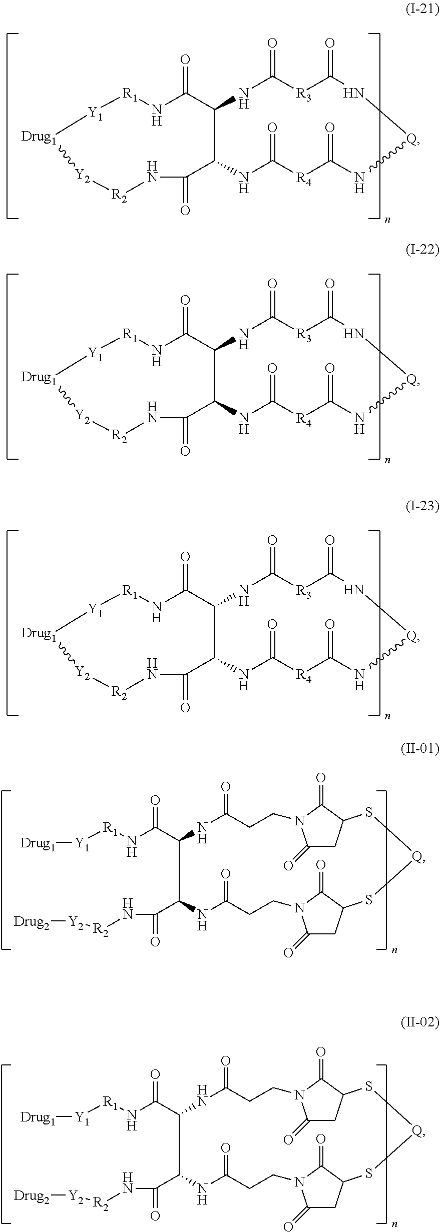 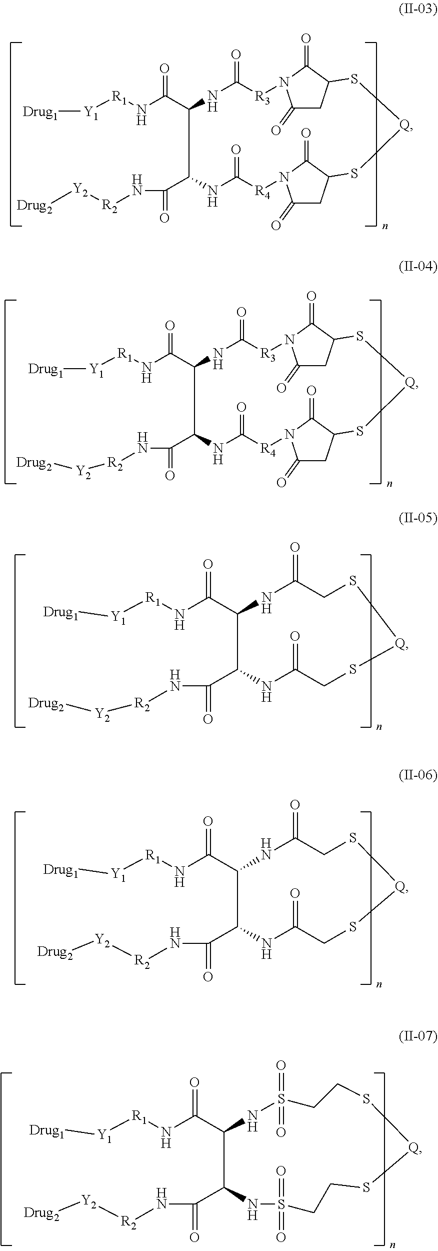 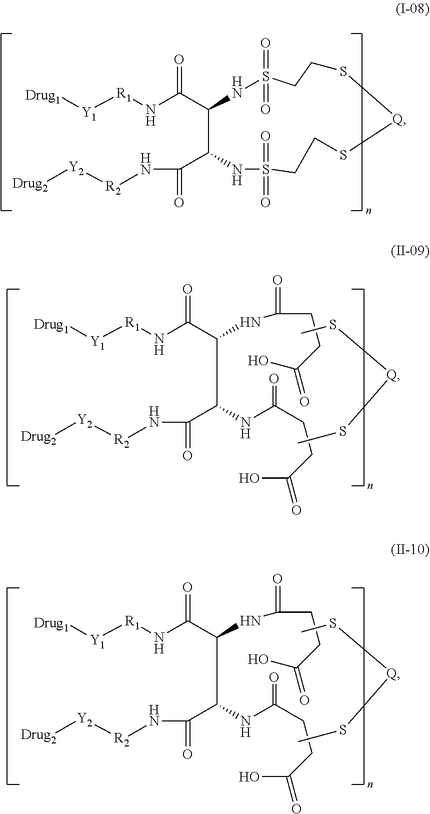 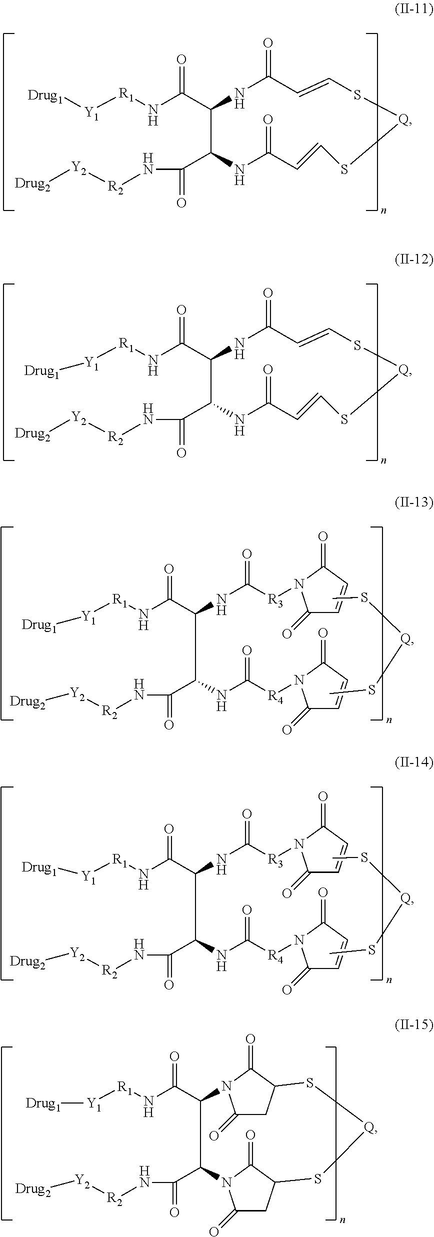 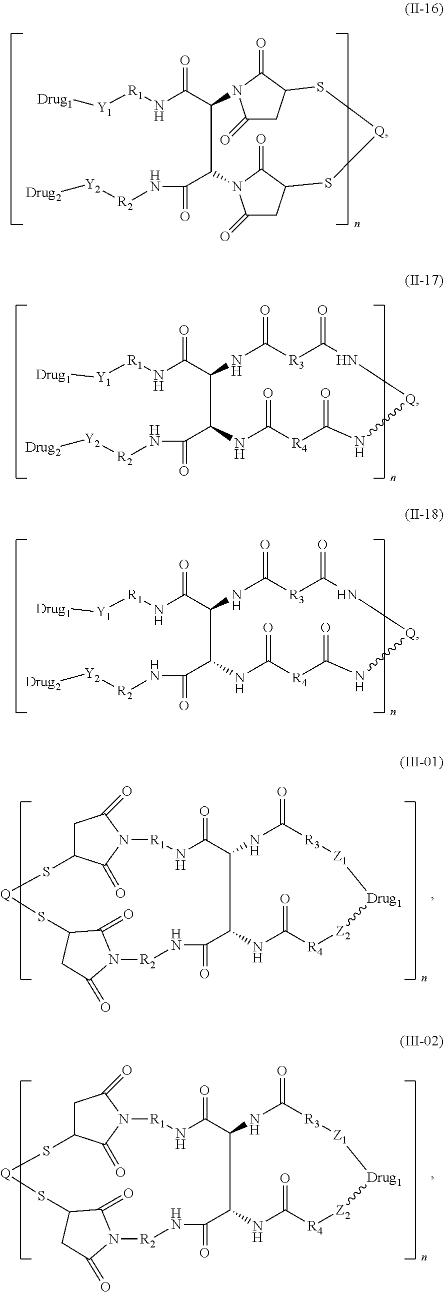 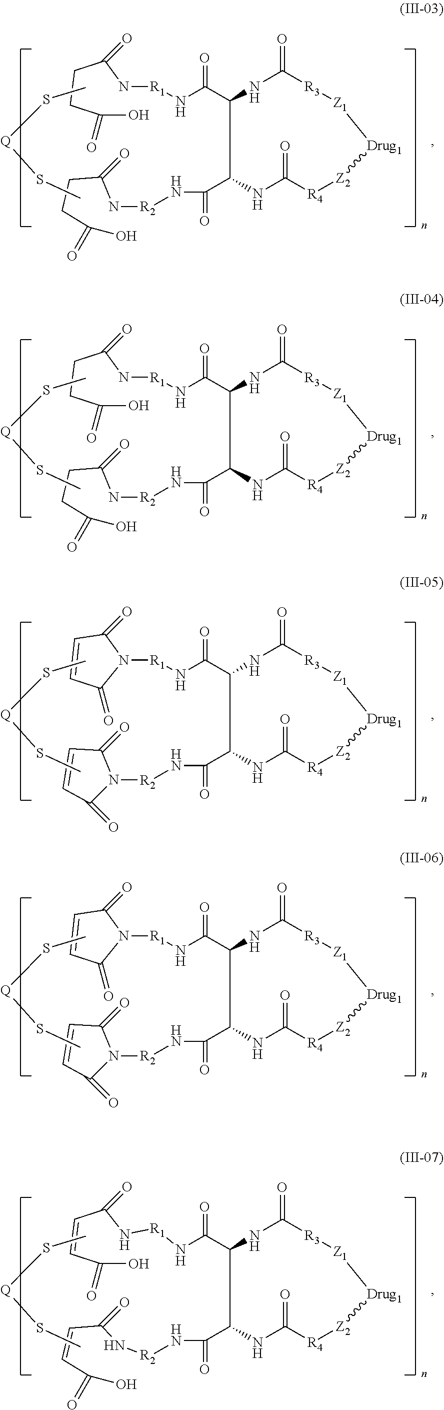 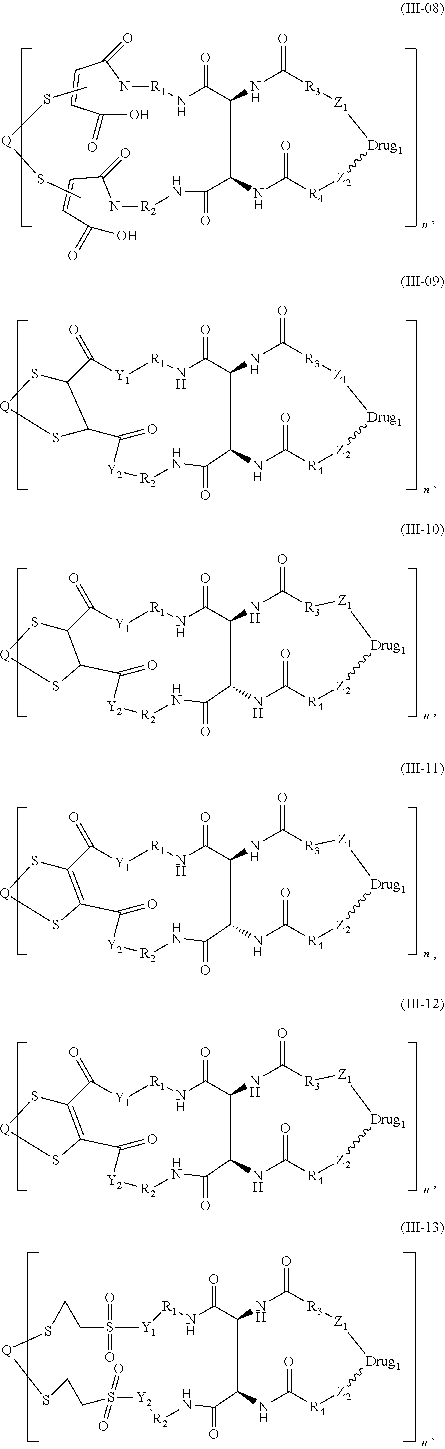 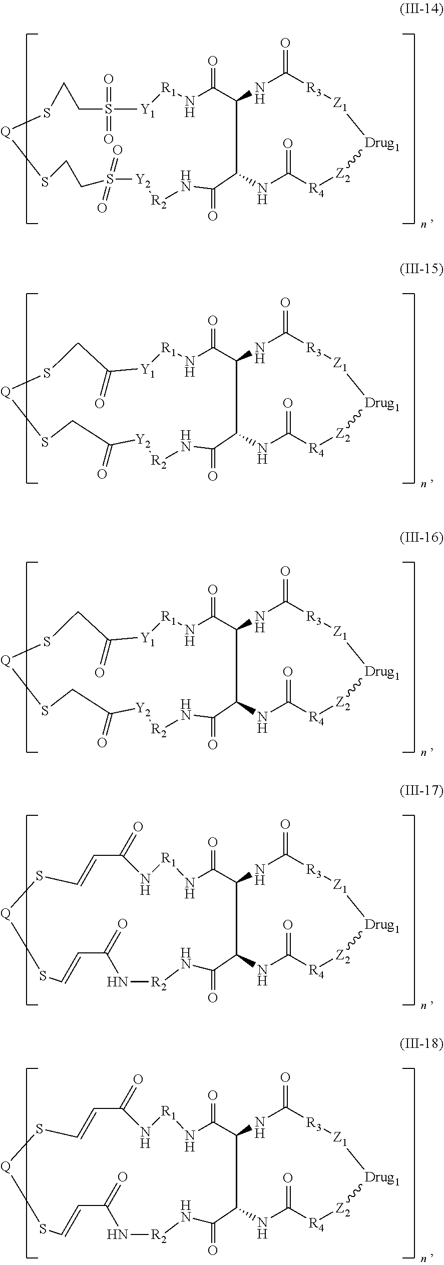 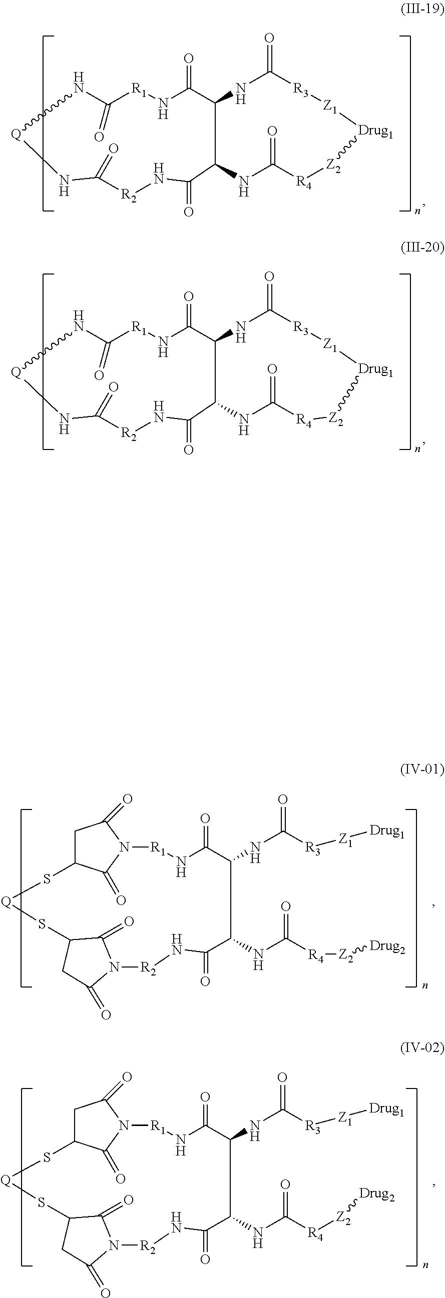 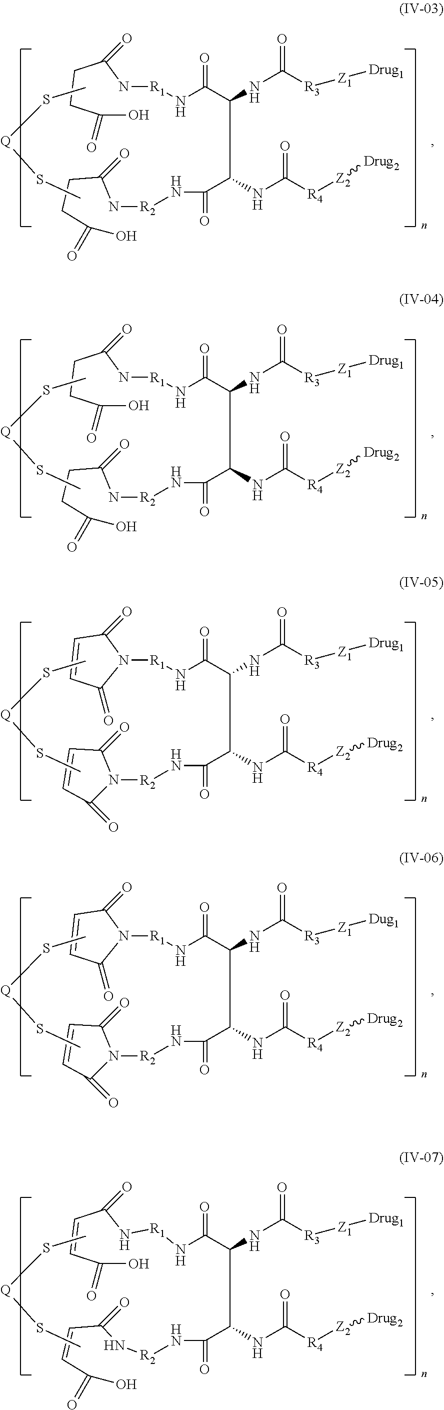 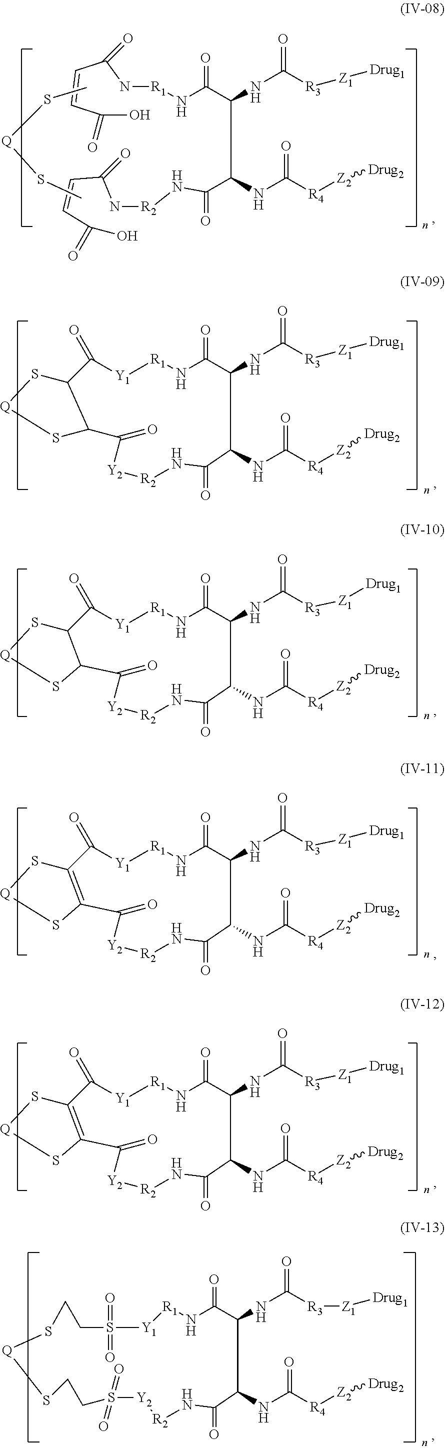 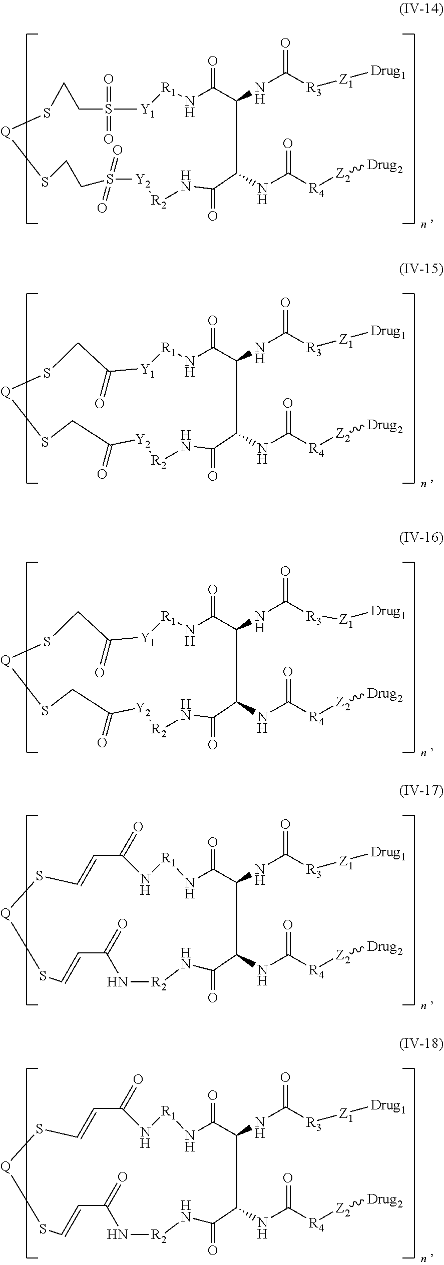 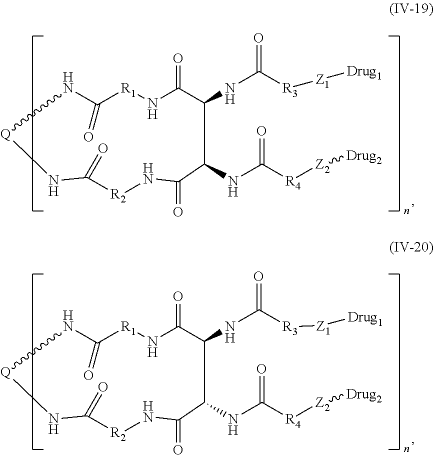
wherein “-----”, “ [symbol] ”, Q, X₁, X₂, Y₁, Y₂, R₁, R₂, R₃, R₄, R₅, R_{5′}, Z₁, Z₂, Drug₁ and Drug₂ are defined the same as in claim 48, or one of Drug₁ and Drug₂ can be independently absent but may not be absent at the same time.

50. A method for preparing the conjugate compound according to claim 49, wherein the method comprises reacting Lv₁ and/or Lv₂ in a stereoisomeric compound presented by Formula (Va), (Vb), (Vc), (VIa), (VIb), (VIc), (VIIa), (VIIb), (VIIc), (VIIIa), (VIIIb) or (VIIIc) with two or more function groups of a cell-binding molecule simultaneously or sequentially: 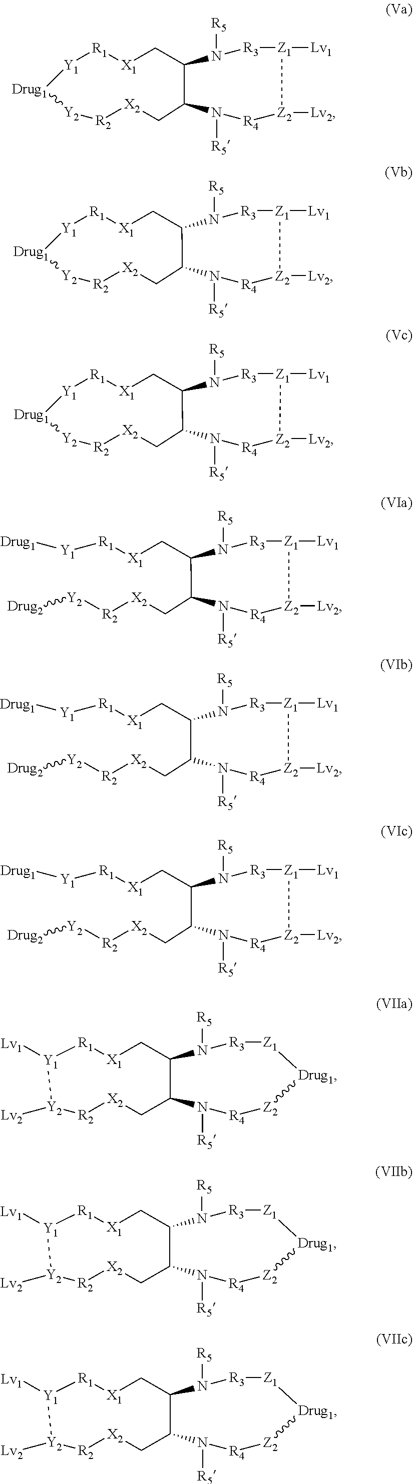 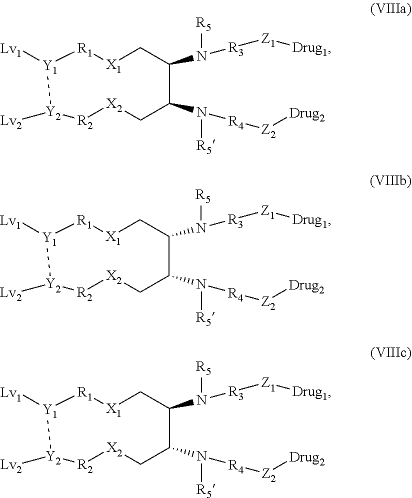
“-----” is a single bond, or a double bond, or a triple bond, or absent; provided that when ----- represents a triple bond, Lv₁ and Lv₂ are absent;
“ [symbol] ”, “ [symbol] ”, Drug₁, Drug₂, n, X₁, X₂, Y₁, Y₂, R₁, R₂, R₃, R₄, R₅, R_{5′}, Z₁, and Z₂ are defined the same as in claim 49;
Lv₁ and Lv₂ represent the same or different leaving group that can be reacted with a thiol, amine, carboxylic acid, selenol, phenol or hydroxyl group on a cell-binding molecule; Lv₁ and Lv₂ are independently selected from the group consisting of OH; F; Cl; Br; I; nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, acetyl anhydride, formyl anhydride; or an intermediate molecule generated with a condensation reagent for peptide coupling reactions, or for Mitsunobu reactions, which is selected from the group consisting of EDC (N-(3-Dimethylaminopropyl)-N′-ethylcarbodiimide), DCC (Dicyclohexyl-carbodiimide), N,N′-Diisopropylcarbodiimide (DIC), N-Cyclohexyl-N′-(2-morpholino-ethyl)carbodiimide metho-p-toluenesulfonate (CMC or CME-CDI), 1,1′-Carbonyldiimi-dazole (CDI), TBTU (O-(Benzotriazol-1-yl)-N,N,N′,N′-tetramethyluronium tetrafluoroborate), N,N,N′,N′-Tetramethyl-O-(1H-benzotriazol-1-yl)-uronium hexafluorophosphate (HBTU), (Benzotriazol-1-yloxy)tris(dimethylamino)-phosphonium hexafluorophosphate (BOP), (Benzotriazol-1-yloxy)tripyrrolidinophosphonium hexafluorophosphate (PyBOP), Diethyl cyanophosphonate (DEPC), Chloro-N,N,N′,N′-tetramethylformamidiniumhexafluorophosphate, 1-[Bis(dimethylamino)methylene]-1H-1,2,3-triazolo[4,5-b]pyridinium 3-oxid hexafluorophosphate (HATU), 1-[(Dimethylamino)(morpholino)methylene]-1H-[1,2,3]triazolo[4,5-b]pyridine-1-ium 3-oxide hexafluoro-phosphate (HDMA), 2-Chloro-1,3-dimethyl-imidazolidinium hexafluorophosphate (CIP), Chlorotripyrrolidinophosphonium hexafluorophosphate (PyCloP), Fluoro-N,N,N′,N′-bis(tetramethylene)formamidinium hexafluorophosphate (BTFFH), N,N,N′,N′-Tetramethyl-S-(1-oxido-2-pyridyl)thiuronium hexafluorophosphate, O-(2-Oxo-1(2H)pyridyl)-N,N,N′,N′-tetramethyluronium tetrafluoroborate (TPTU), S-(1-Oxido-2-pyridyl)-N,N,N′,N′-tetramethylthiuronium tetrafluoroborate, O-[(Ethoxycarbonyl)-cyanomethylenamino]-N,N,N′,N′-tetramethyluronium hexafluorophosphate (HOTU), (1-Cyano-2-ethoxy-2-oxoethylidenaminooxy) dimethylamino-morpholino-carbenium hexafluorophosphate (COMU), O-(Benzotriazol-1-yl)-N,N,N′,N′-bis(tetramethylene)uronium hexafluorophosphate (HBPyU), N-Benzyl-N′-cyclohexyl-carbodiimide (with or without polymer-bound), Dipyrrolidino(N-succinimidyl-oxy)carbenium hexafluoro-phosphate (HSPyU), Chlorodipyrrolidinocarbenium hexafluorophosphate (PyCIU), 2-Chloro-1,3-dimethylimidazolidinium tetrafluoroborate (CIB), (Benzotriazol-1-yloxy)dipiperidino-carbenium hexafluorophosphate (HBPipU), O-(6-Chlorobenzotriazol-1-yl)-N,N,N′,N′-tetramethyluronium tetrafluoroborate (TCTU), Bromotris(dimethylamino)-phosphonium hexafluorophosphate (BroP), Propylphosphonic anhydride (PPACA, T3P®), 2-Morpholinoethyl isocyanide (MEI), N,N,N′,N′-Tetramethyl-O—(N-succinimidyl)uronium hexafluorophosphate (HSTU), 2-Bromo-1-ethyl-pyridinium tetrafluoroborate (BEP), O-[(Ethoxycarbonyl)cyano-methylenamino]-N,N,N′,N′-tetra-methyluronium tetrafluoroborate (TOTU), 4-(4,6-Dimethoxy-1,3,5-triazin-2-yl)-4-methylmorpholiniumchloride (MMTM, DMTMM), N,N,N′,N′-Tetramethyl-O—(N-succinimidyl)uronium tetrafluoroborate (TSTU), O-(3,4-Dihydro-4-oxo-1,2,3-benzotriazin-3-yl)-N,N,N′,N′-tetramethyluronium tetrafluoro-borate (TDBTU),1,1′-(Azodicarbonyl)-dipiperidine (ADD), Di-(4-chlorobenzyl)azodicarboxylate (DCAD), Di-tert-butyl azodicarboxylate (DBAD),Diisopropyl azodicarboxylate (DIAD), and Diethyl azodicarboxylate (DEAD), or Lv₁ and Lv₂ can be an anhydride, formed by acid themselves or formed with other C₁-C₈ acid anhydrides;
or Lv₁ and Lv₂ can be independently, a halide (fluoride, chloride, bromide, and iodide), methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluorophenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or carbon-oxygen), or one of the following structure: 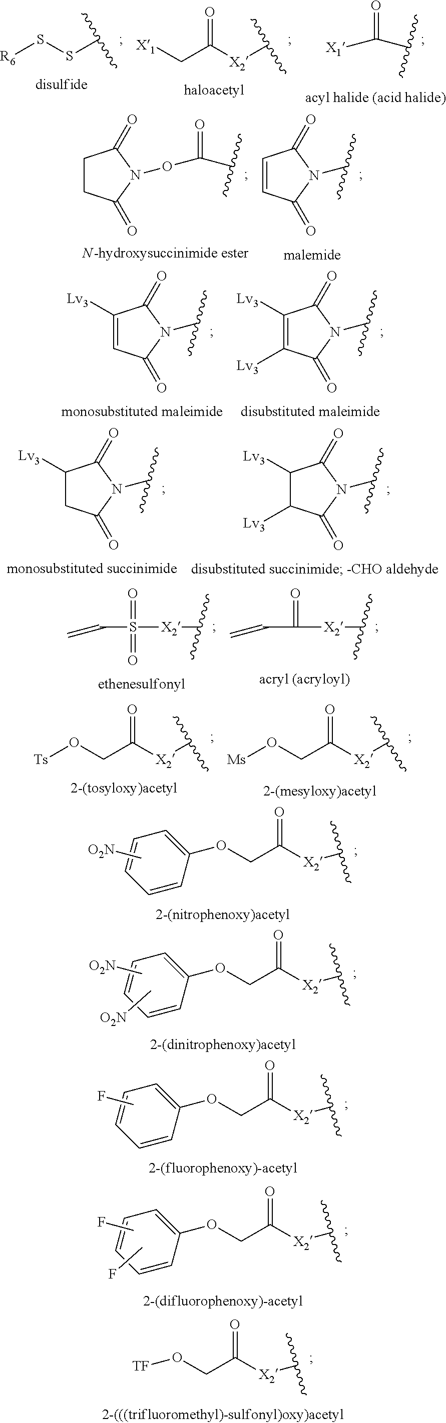 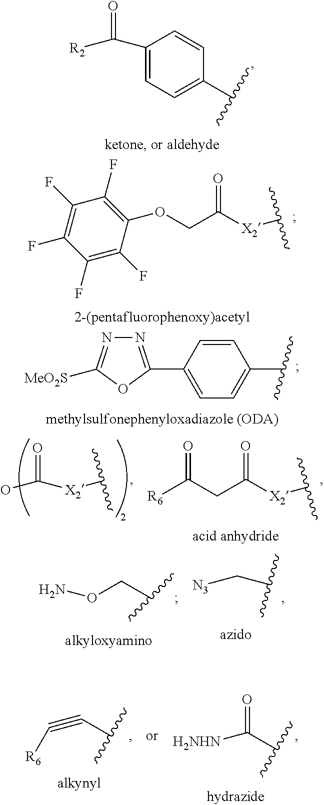
wherein X₁′ is F, Cl, Br, I or Lv₃; X₂′ is O, NH, N(R₁), or CH₂; R₃ is independently H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from the group consisting of F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; and 2-ethyl-5-phenylisoxazolium-3′-sulfonate, R, and R₂ are defined above.

51. The method according to claim 50, wherein the stereoisomeric compound has a structure represented by Formula (V-01), (V-02), (V-03), (V-04), (V-05), (V-06), (V-07), (V-08), (V-09), (V-10), (V-11), (V-12), (V-13), (V-14), (V-15), (V-16), (V-17), (V-18), (V-19), (V-20), (V-21), (V-22), (V-23), (VI-01), (VI-02), (VI-03), (VI-04), (VI-05), (VI-06), (VI-07), (VI-08), (VI-09), (VI-10), (VI-11), (VI-12), (VI-13), (VI-14), (VI-15), (VI-16), (VI-17), (VI-18), (VII-01), (VII-02), (VII-03), (VII-04), (VII-05), (VII-06), (VII-07), (VII-08), (VII-09), (VII-10), (VII-11), (VII-12), (VII-13), (VII-14), (VII-15), (VII-16), (VII-17), (VII-18), (VII-19), (VII-20), (VIII-01), (VIII-02), (VIII-03), (VIII-04), (VIII-05), (VIII-06), (VIII-07), (VIII-08), (VIII-09), (VIII-10), (VIII-11), (VIII-12), (VIII-13), (VIII-14), (VIII-15), (VIII-16), (VIII-17), (VIII-18), (VIII-19), or (VIII-20) below: 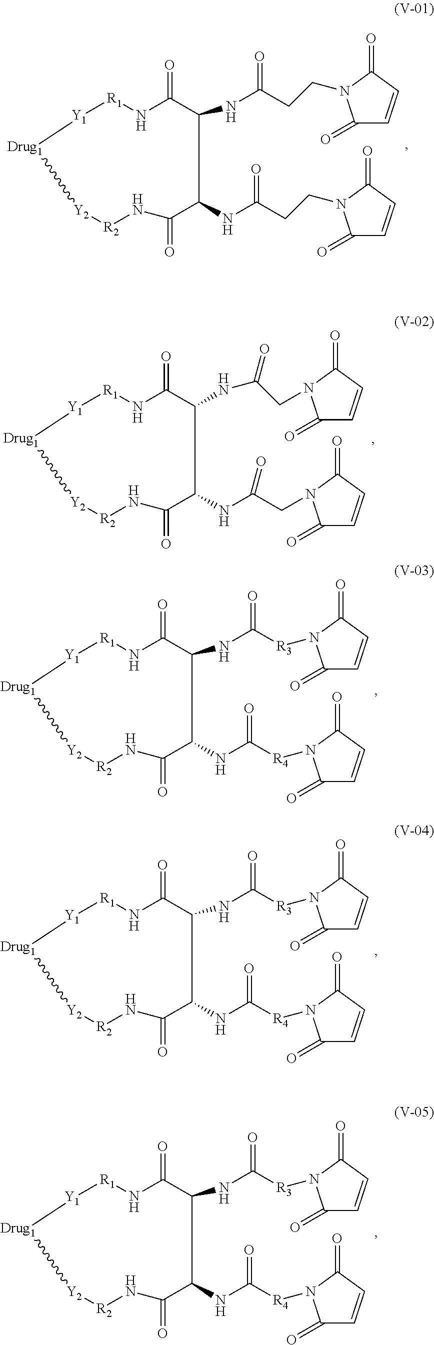 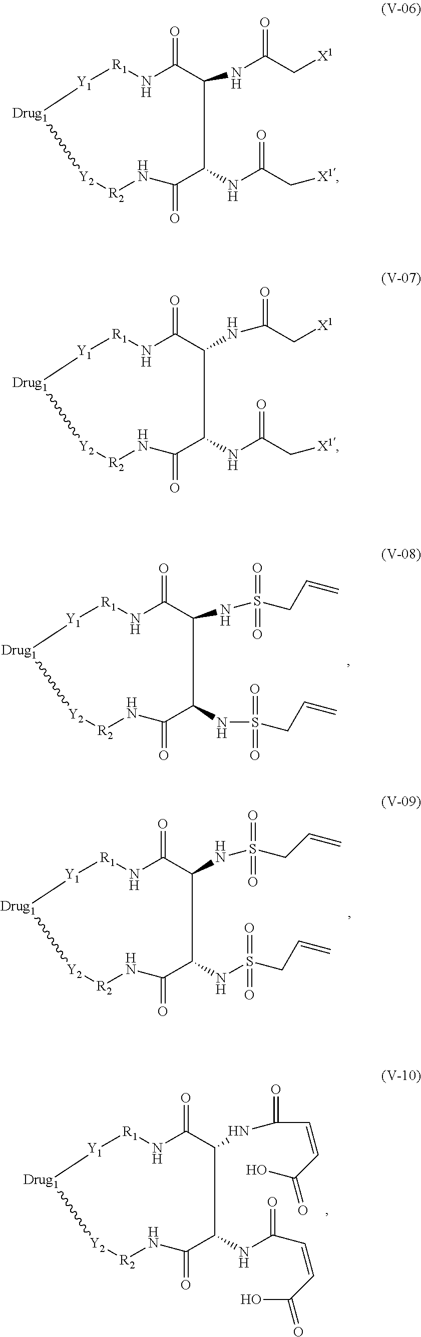 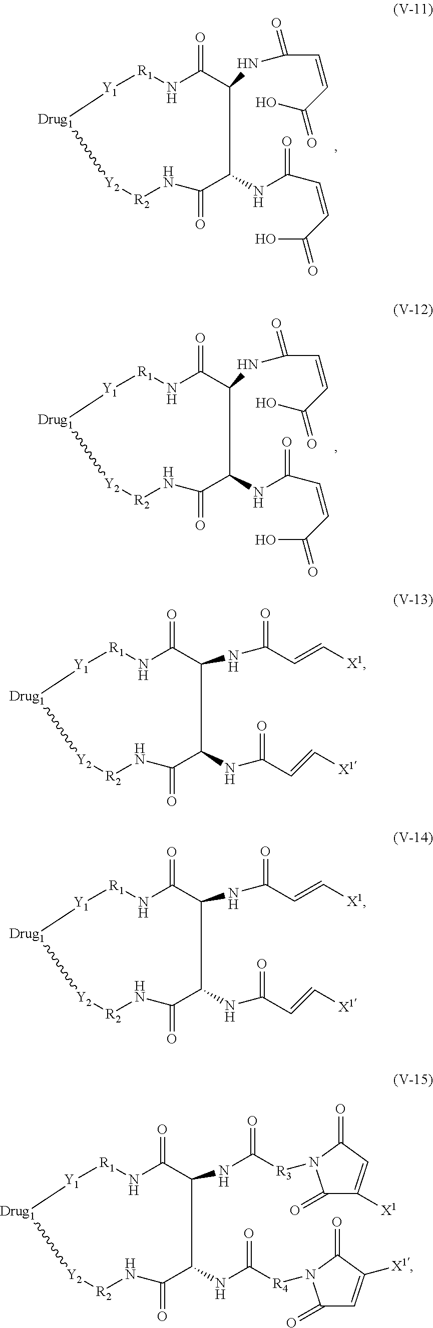 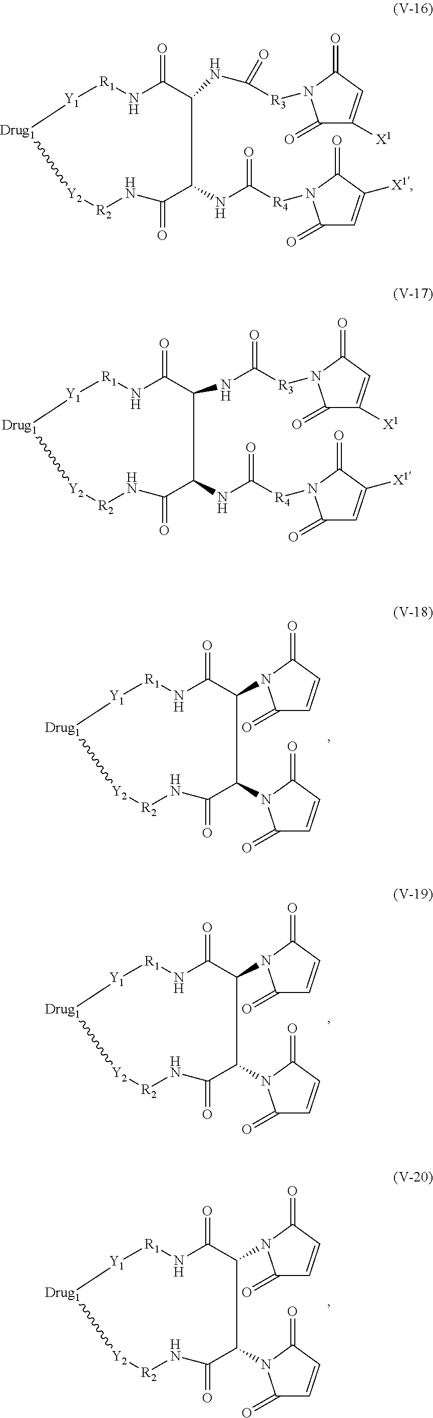 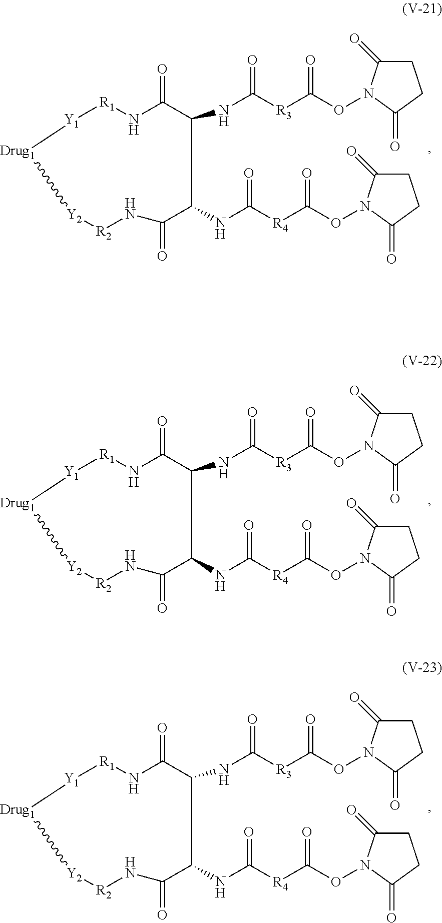 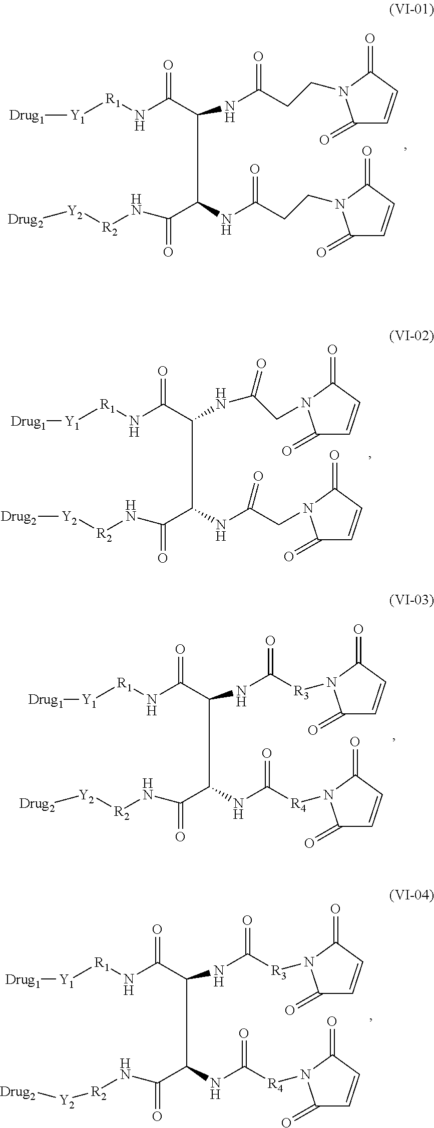 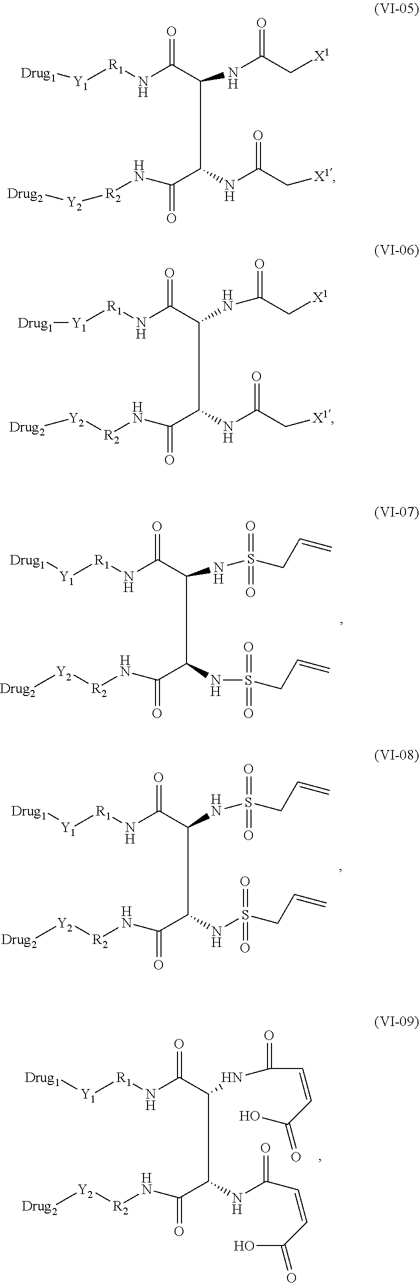 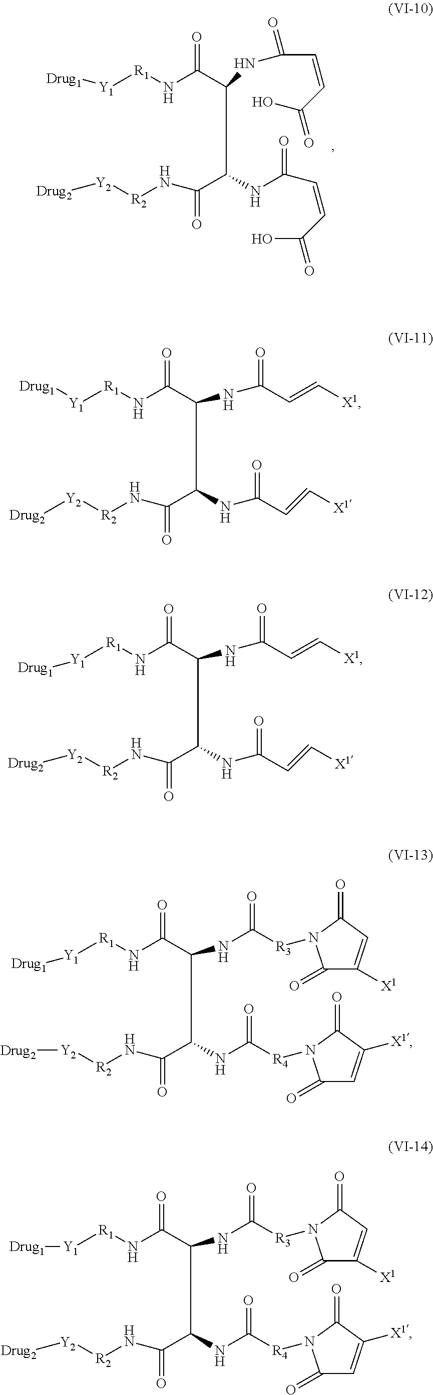 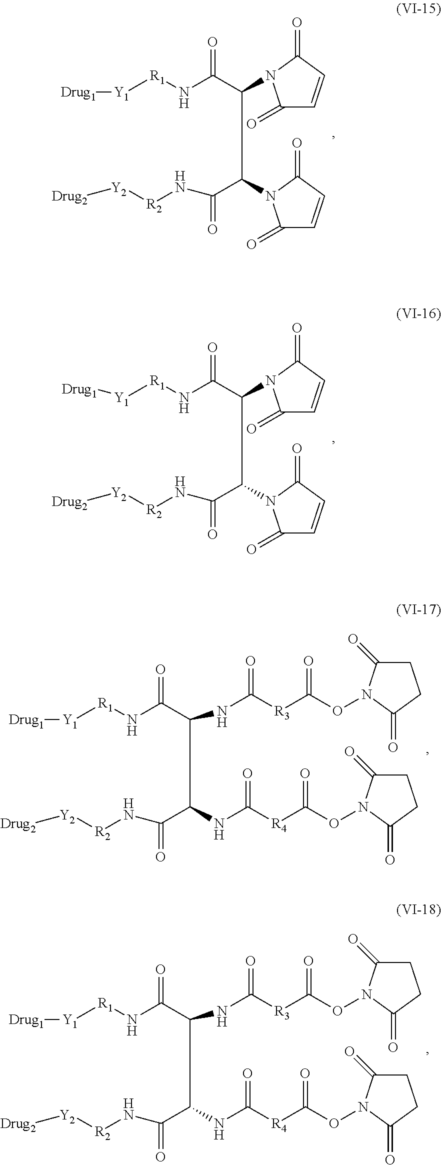 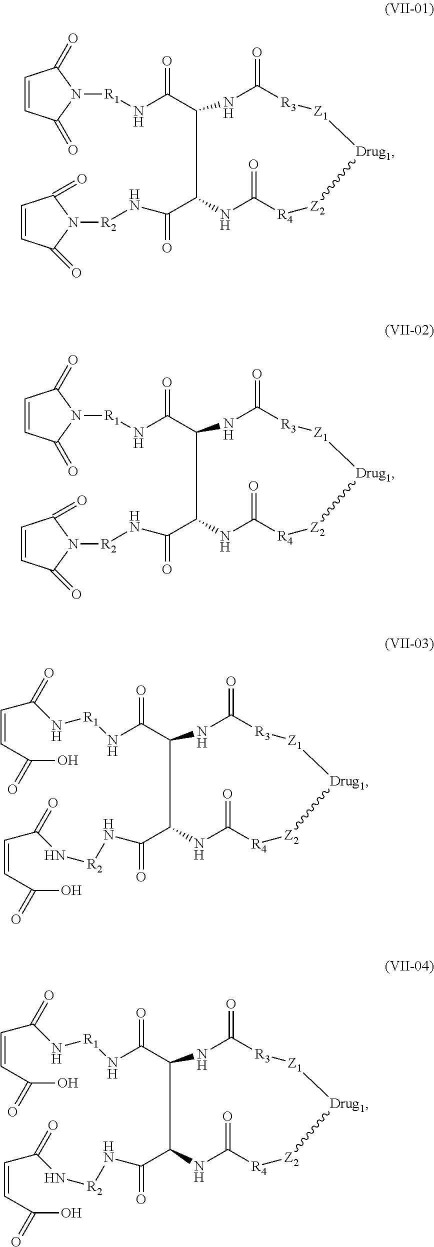 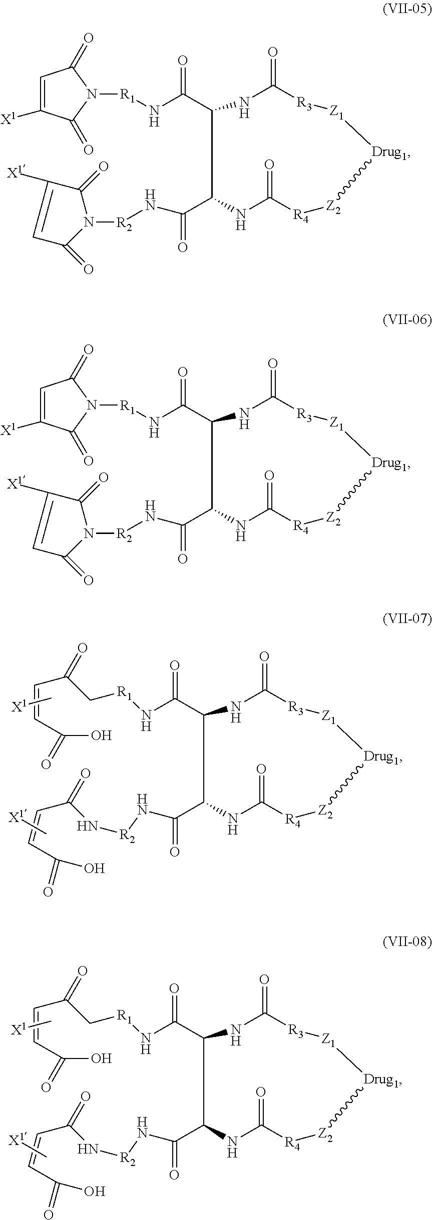 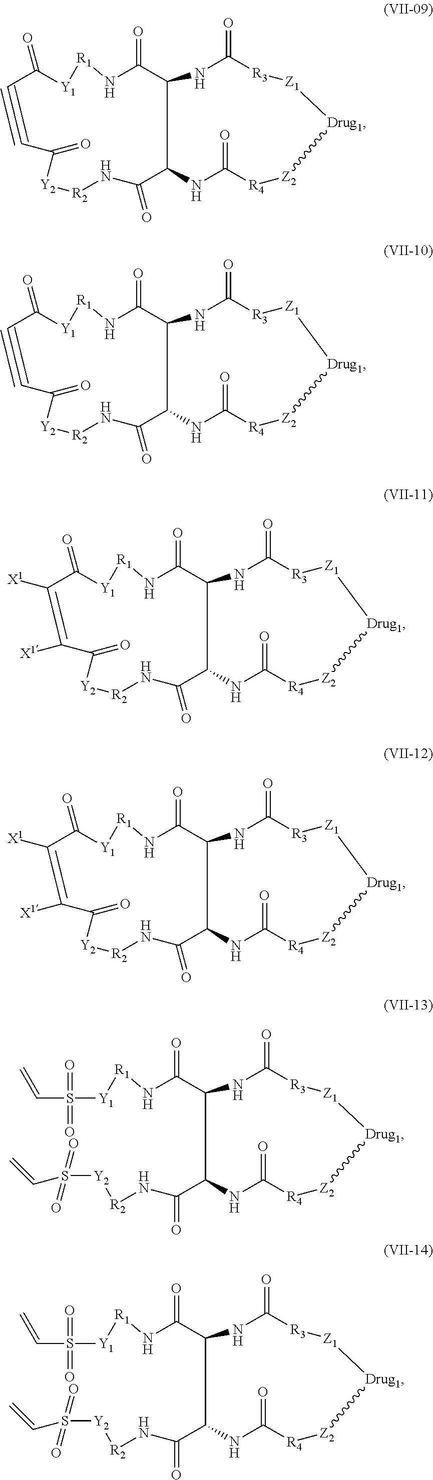 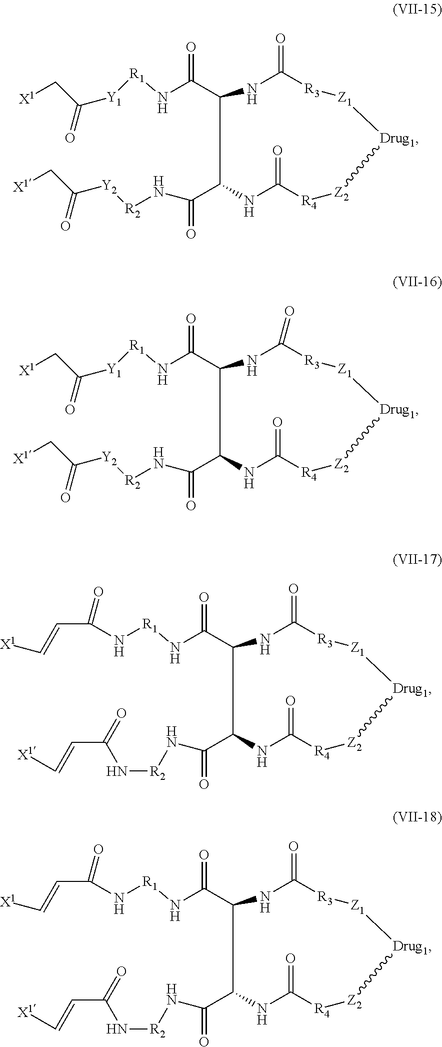 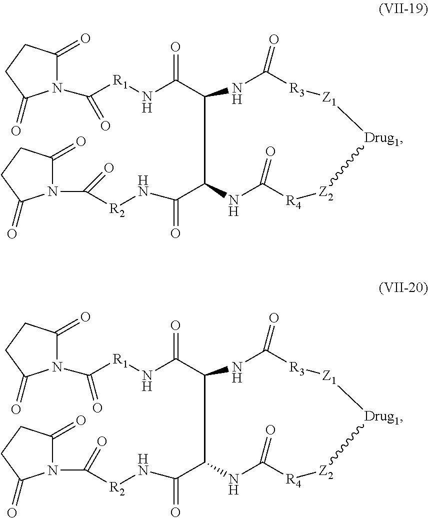 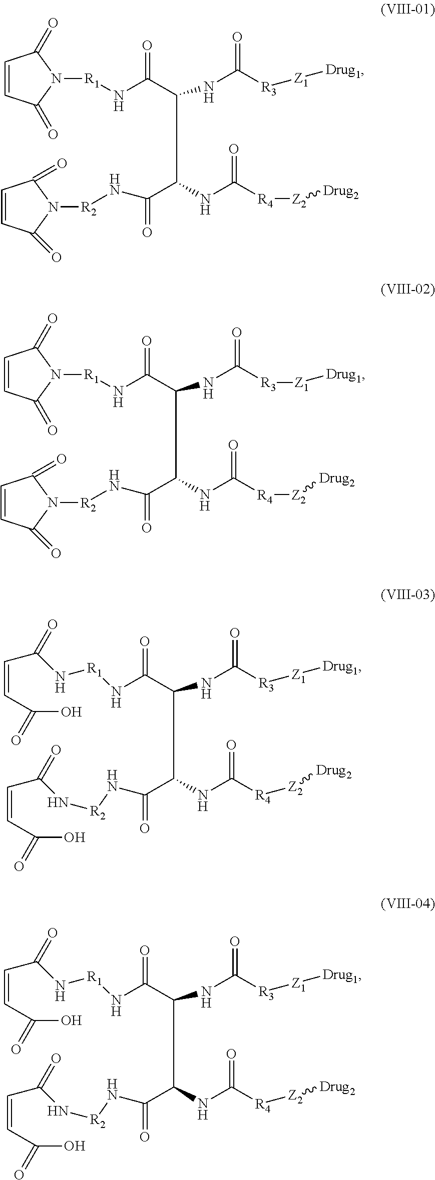 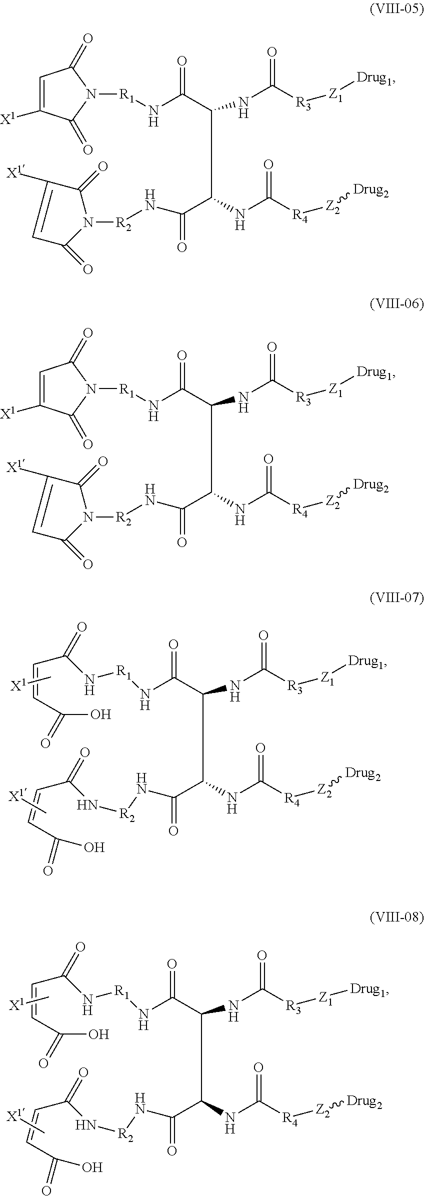 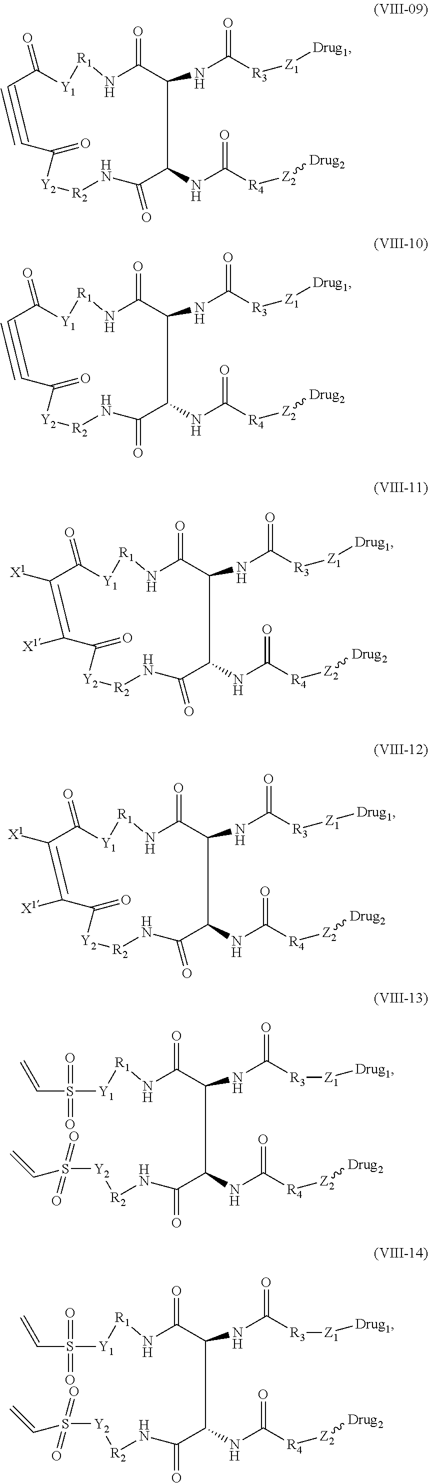 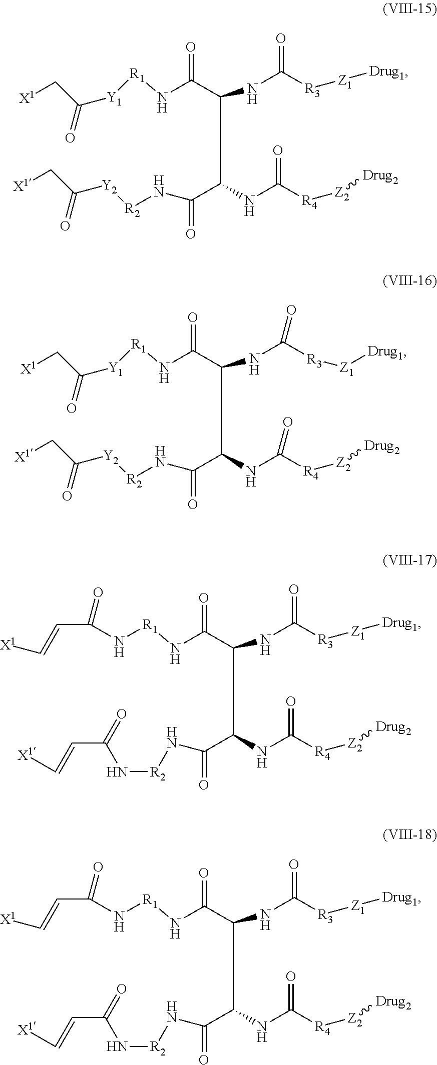 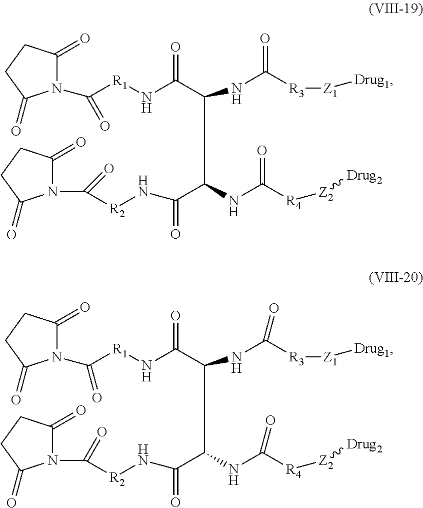
wherein “----”, “ [symbol] ”, Q, X₁, X₂, Y₁, Y₂, R₁, R₂, R₃, R₄, R₅, R_{5′}, Z₁, Z₂, Drug₁ and Drug₂ are defined the same as in claim 50; X¹ and X^{1′} are independently H, F, Cl, Br, I, OTs, OMs, OTf, N₃, CHO, —C═CH, —C═C—, ArC(═O)R₁, C(═O)NHNH₂, —O—NH₂, nitrophenol; N-hydroxysuccinimide (NHS); phenol; dinitrophenol; pentafluorophenol; tetrafluorophenol; difluorophenol; mono-fluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with acetyl anhydride or formyl anhydride; O—NHS (O—N-hydrosuccinimide), O-imidazole, O-triazole, O-tetrazole, O—Ar, O—ArNO₂, O—Ar(NO₂)₂, O—ArF₄, O—ArF₃, O—ArF₅, O—ArF₂, O—ArF, O—ArCl₄, O—ArCl₃, O—ArCl₅, O—ArCl₂, O—ArCl, O—ArSO₃H, O—ArOPO₃H₂, O—Ar(NO₂)COOH, S—Ar(NO₂)₂COOH, O-pyridine, O-nitrophenol, O-dinitrophenol, O-pentafluorophenol, O-tetrafluorophenol, O-trifluorophenol, O-difluorophenol, O-fluorophenol, O-pentachlorophenol, O-tetrachlorophenol, O-trichloro-phenol, O-dichlorophenol, O-chlorophenol, O-pyridine, O-nitropyridine, O-dinitropyridine, O—C₁-C₈ alkyl, O-triflate, O-benzotriazole, S—Ar, S—ArNO₂, S—Ar(NO₂)₂, S—ArF₄, S—ArF₃, S—ArF₅, S—ArF₂, S—ArF, S—ArCl₄, S—ArCl₃, S—ArCl₅, S—ArCl₂, S—ArCl, S—ArSO₃H, S—ArOPO₃H₂, S—Ar(NO₂)COOH, S—Ar(NO₂)₂COOH, S-pyridine, S—S-pyridine, S-nitropyridine, S-dinitropyridine, S—C₁-C₈ alkyl, S—S—C₁-C₈ alkyl, S-triflate, S-benzotriazole, wherein Ar is C₃-C₈ aromatic ring; or an intermediate molecule generated with a condensation reagent for peptide coupling reactions, or for Mitsunobu reactions.

52. A method for preparing the conjugate compound according to claim 48, wherein the method comprises reacting two or more function groups of a cytotoxic molecule simultaneously or sequentially with Lv₁′ and/or Lv₂′ of a compound represented by Formula (IX-01), (IX-02), (IX-03), (IX-04), (IX-05), (IX-06), (IX-07), (IX-08), (IX-09), (IX-10), (IX-11), (IX-12), (IX-13), (IX-14), (IX-15), (IX-16), (IX-17), (IX-18), (IX-19), (IX-20), (IX-21), (IX-22), (IX-23), (X-01), (X-02), (X-03), (X-04), (X-05), (X-06), (X-07), (X-08), (X-09), (X-10), (X-11), (X-12), (X-13), (X-14), (X-15), (X-16), (X-17), (X-18), (X-19), or (X-20) below: 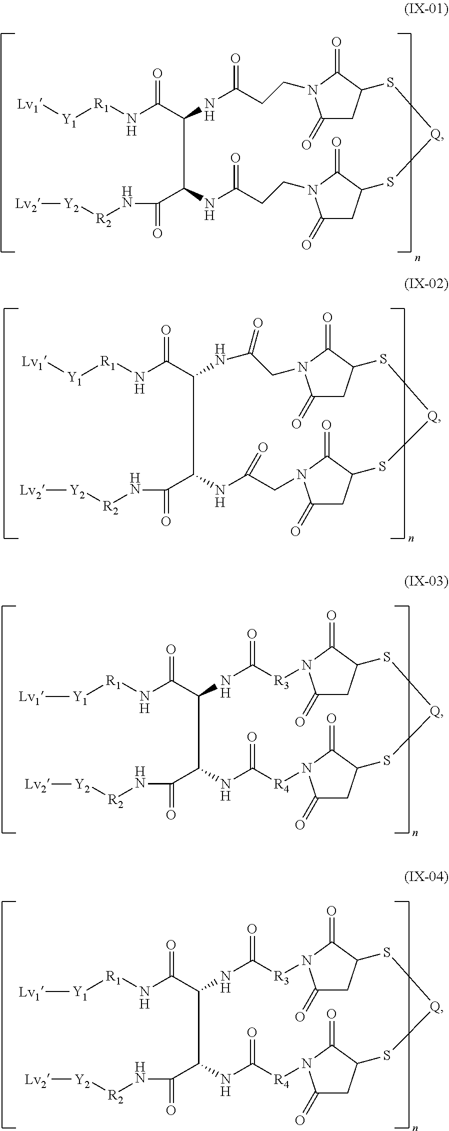 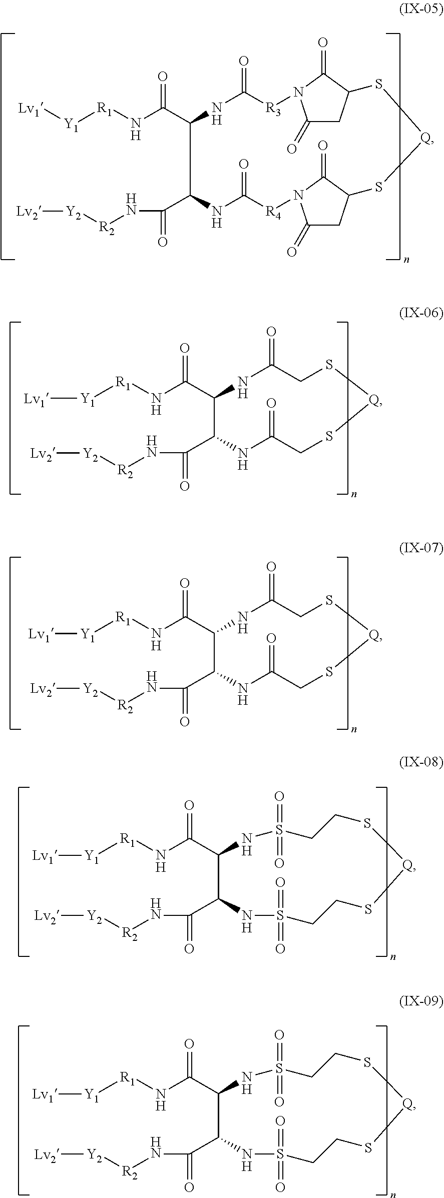 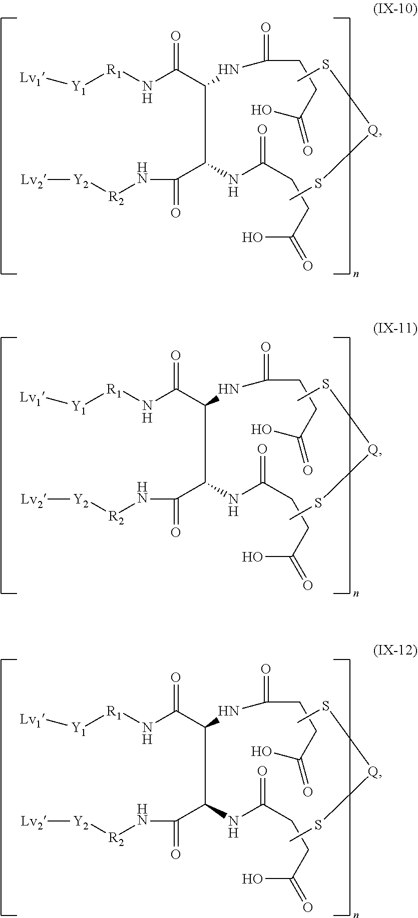 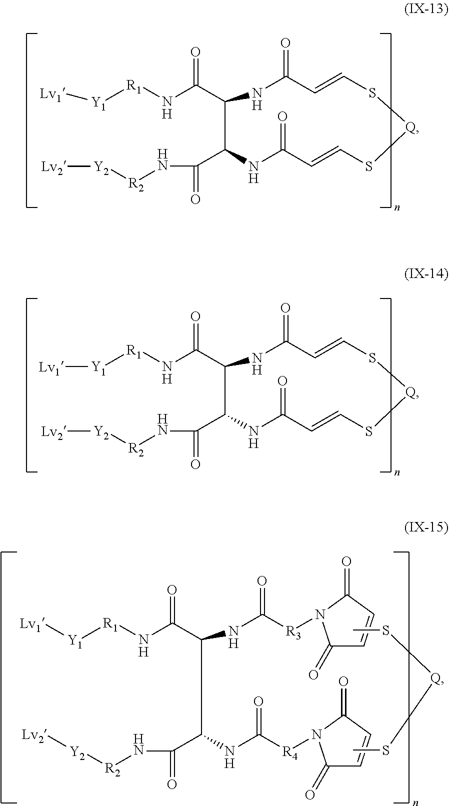 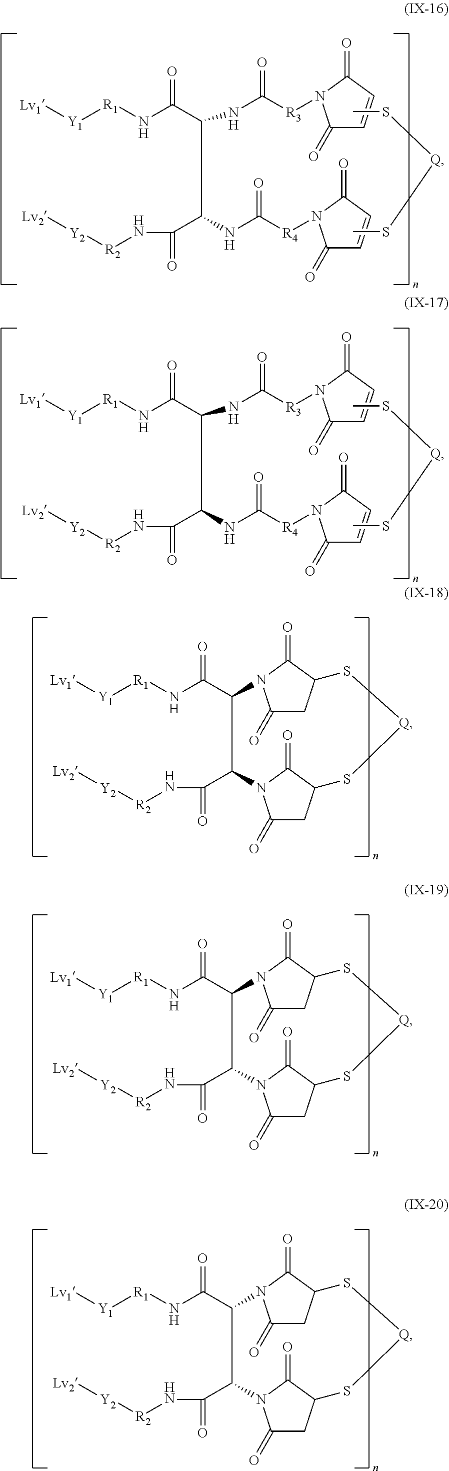 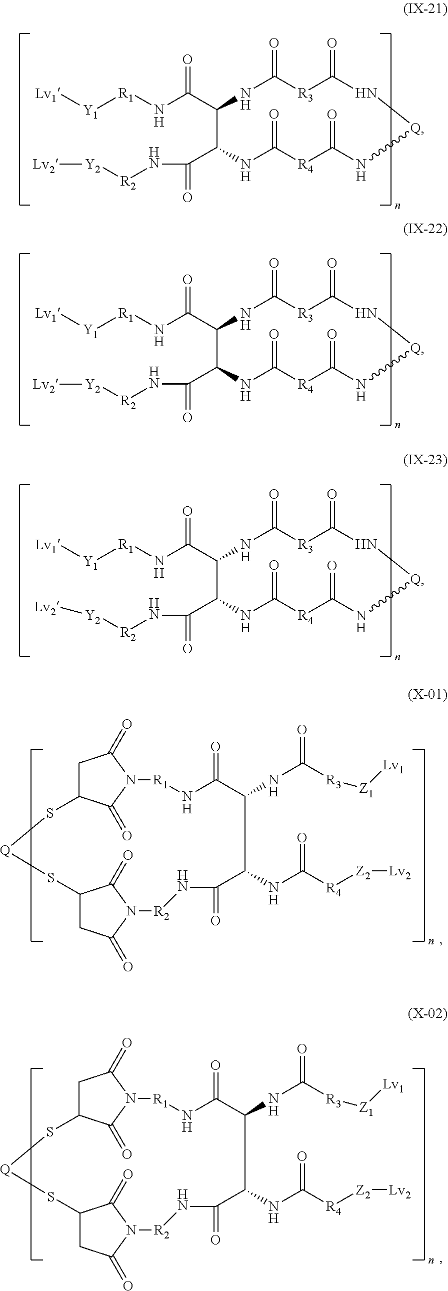 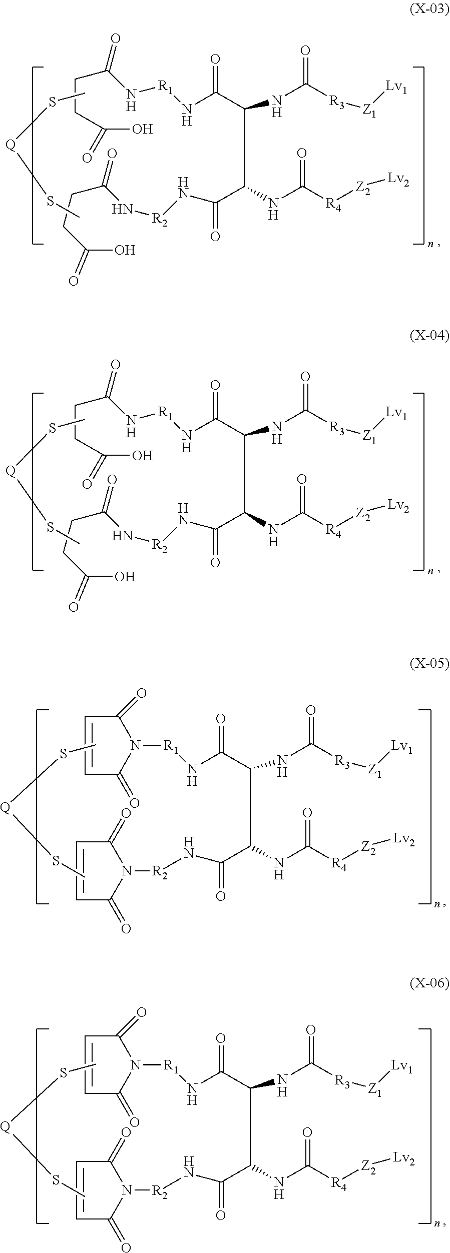 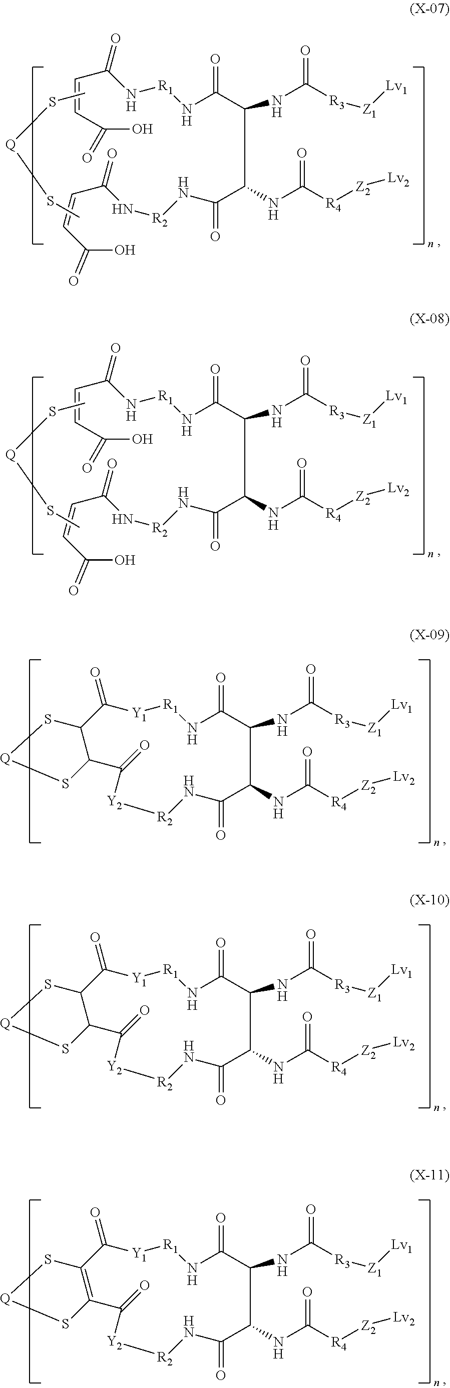 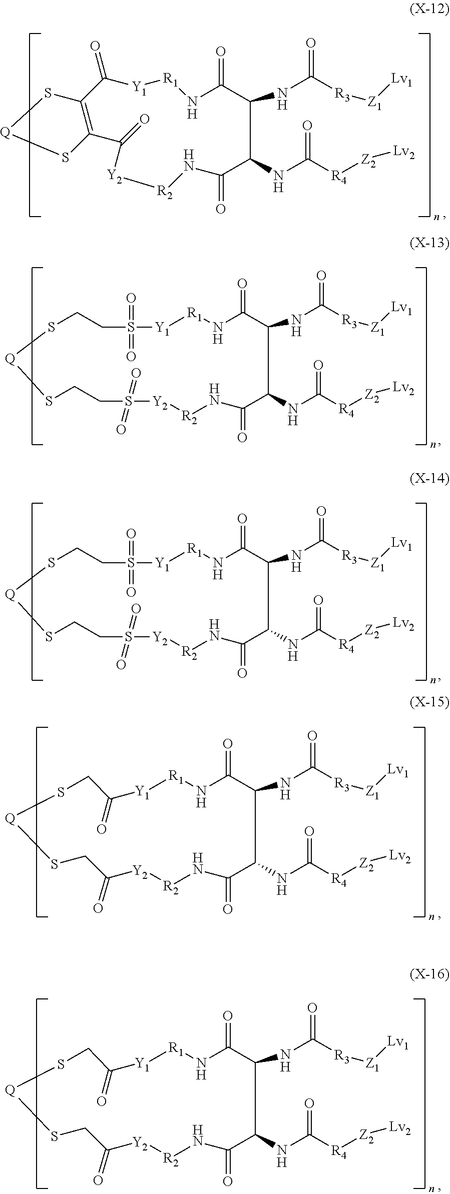 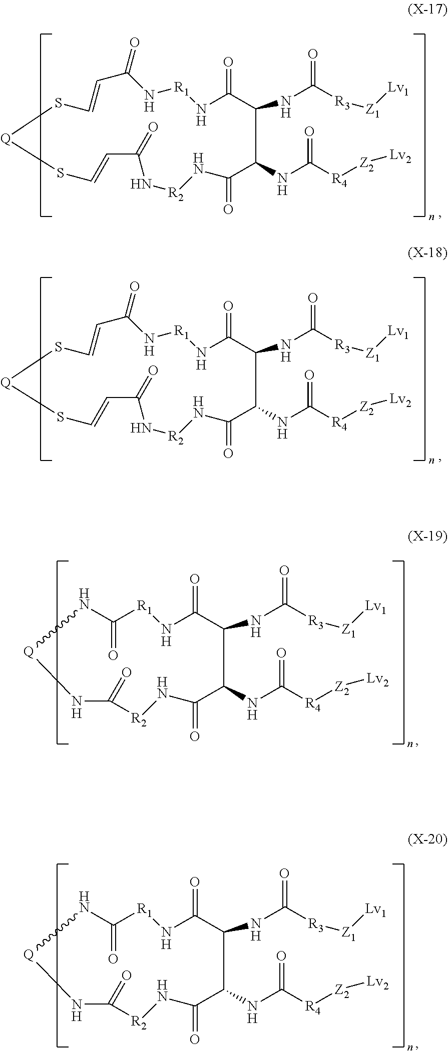
wherein “-----”, “ [symbol] ”, Q, X₁, X₂, Y₁, Y₂, R₁, R₂, R₃, R₄, R₅, R_{5′}, Z₁, Z₂, Lv₁, Lv₂, Lv₁′, and Lv₂′ are defined as in claim 48, one of Drug, and Drug₂ can be independently absent but may not be absent at the same time.

53. A method for preparing the conjugate compound according to claim 48, wherein the method comprises reacting a compound represented by Formula (XI-01), (XI-02), (XI-03), (XI-04), (XI-05), (XI-06), (XI-07), (XI-08), (XI-09), (XI-10), (XI-11), (XI-12), (XI-13), (XI-14), (XI-15), (XI-16), (XI-17), (XI-18), (XII-01), (XII-02), (XII-03), (XII-04), (XII-05), (XII-06), (XII-07), (XII-08), (XII-09), (XII-10), (XII-11), (XII-12), (XII-13), (XII-14), (XII-15), (XII-16), (XII-17), (XII-18), (XII-19), (XII-20), (XII-21), (XII-22), (XII-23), or (XII-24), with a cytotoxic molecule and a cell-binding molecule independently, or simultaneously, or sequentially: 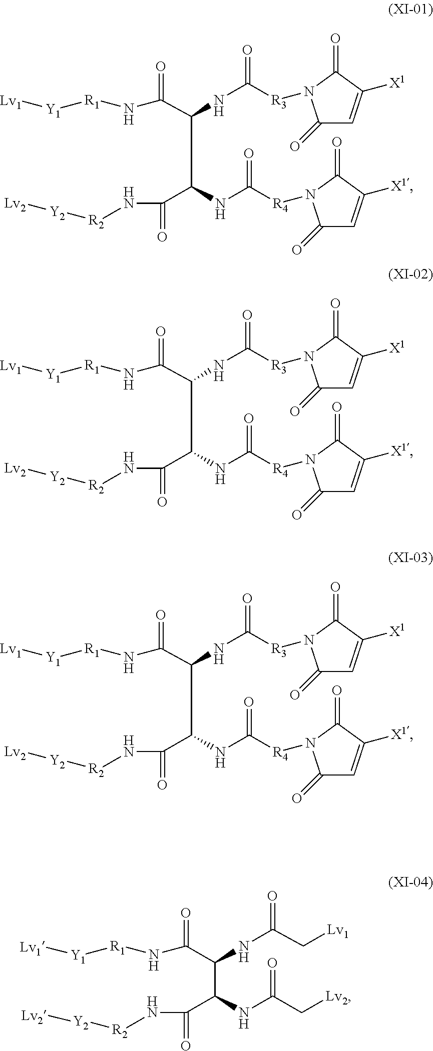 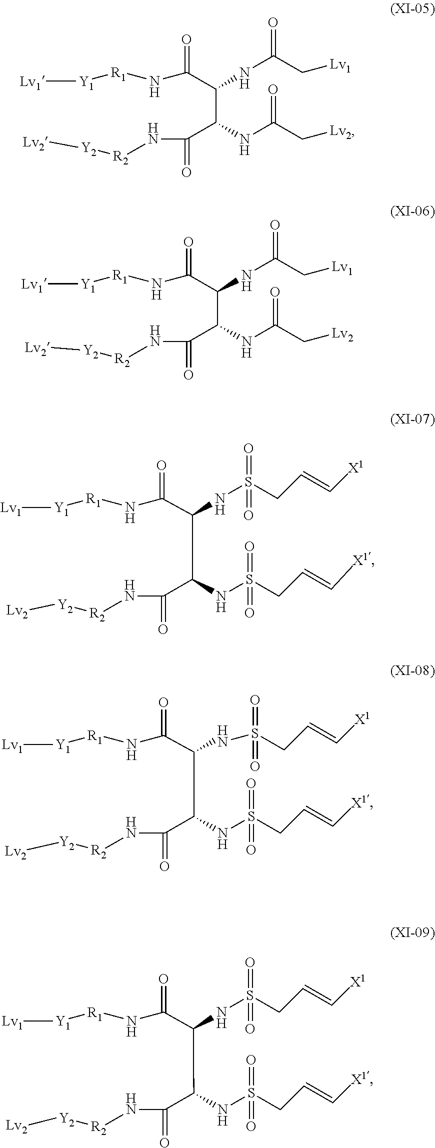 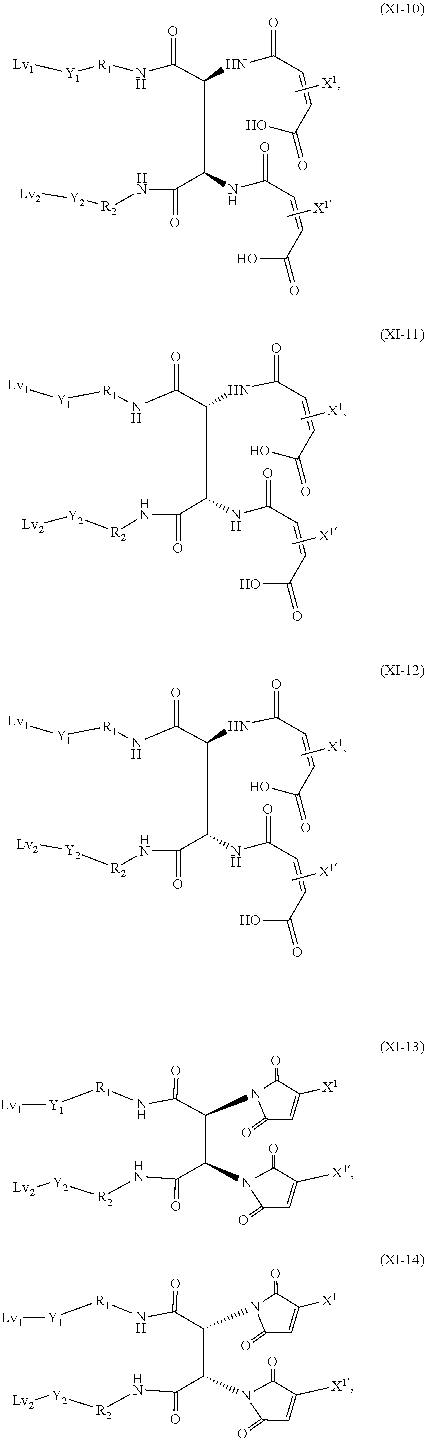 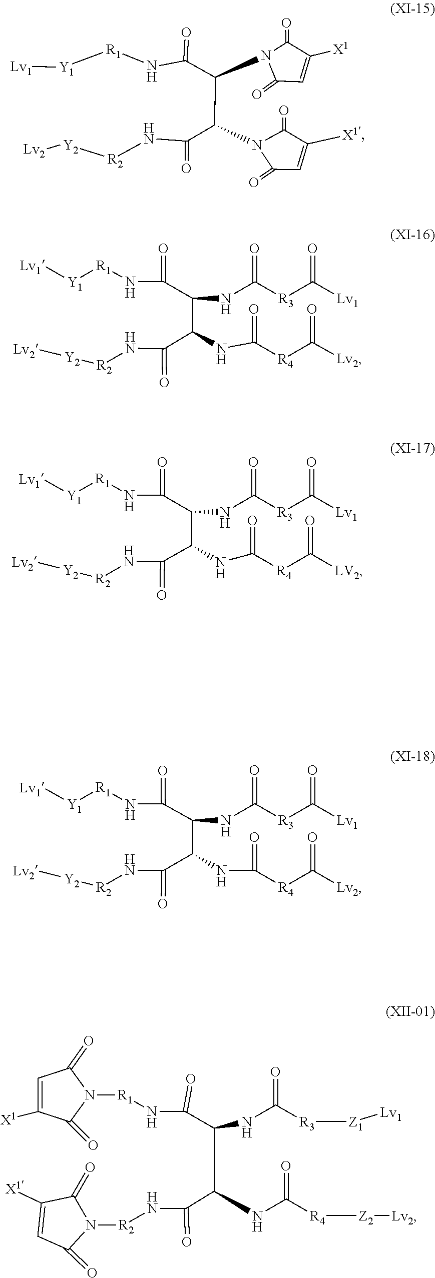 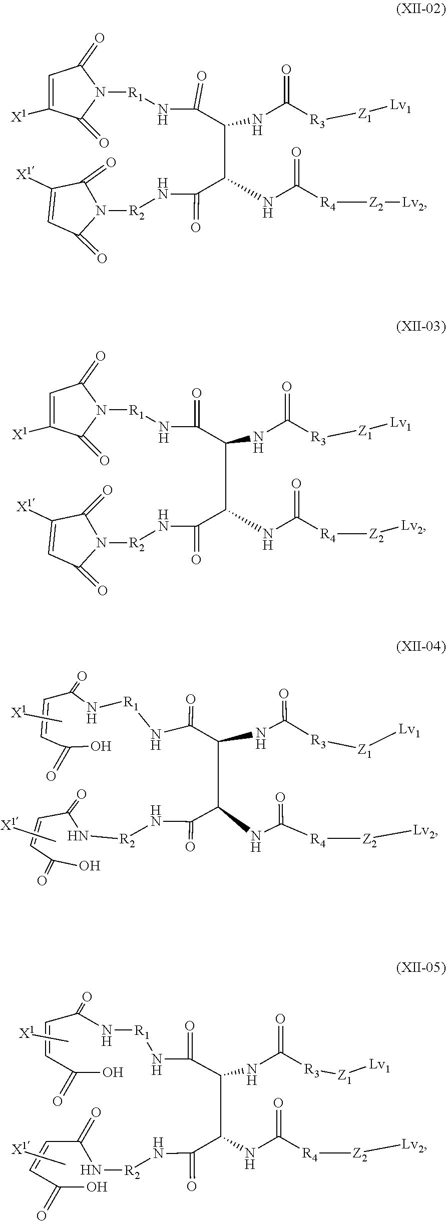 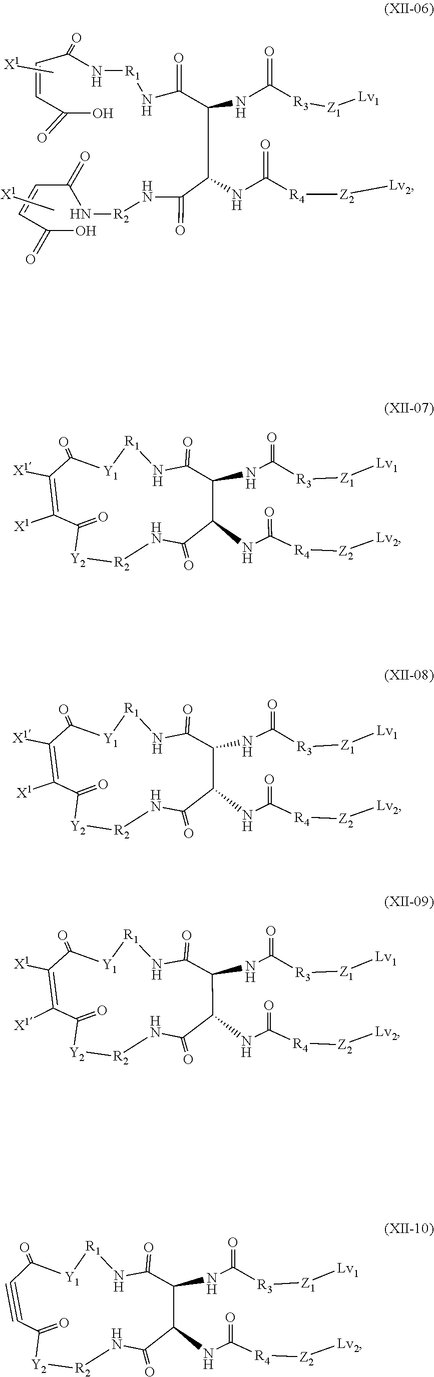 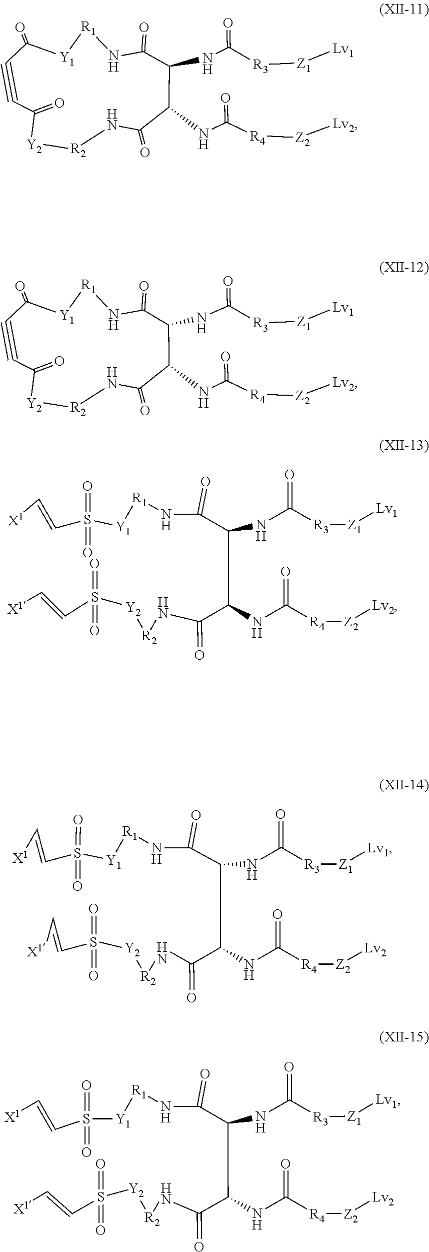 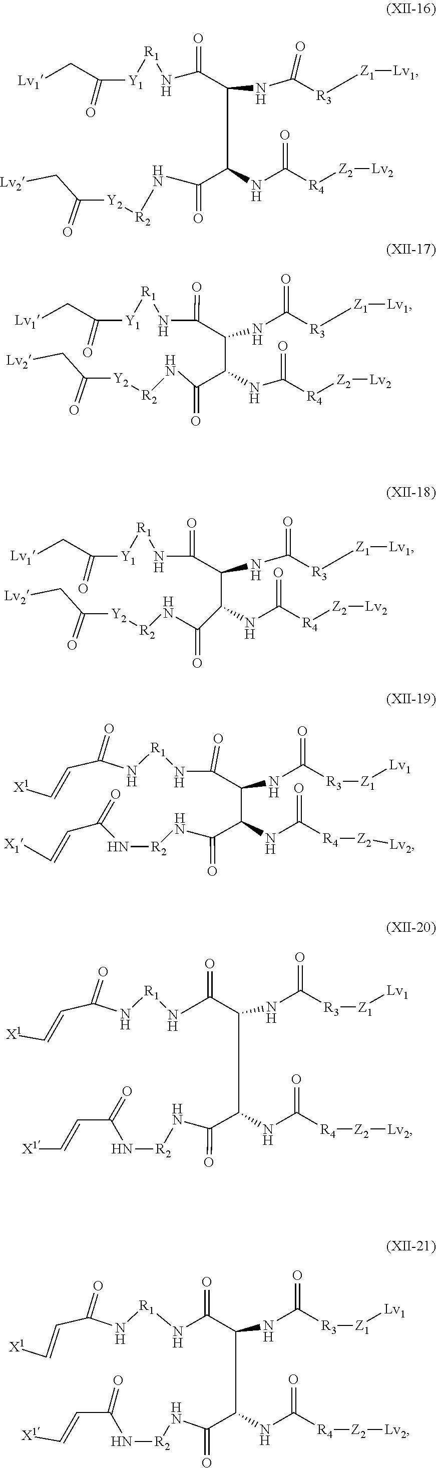 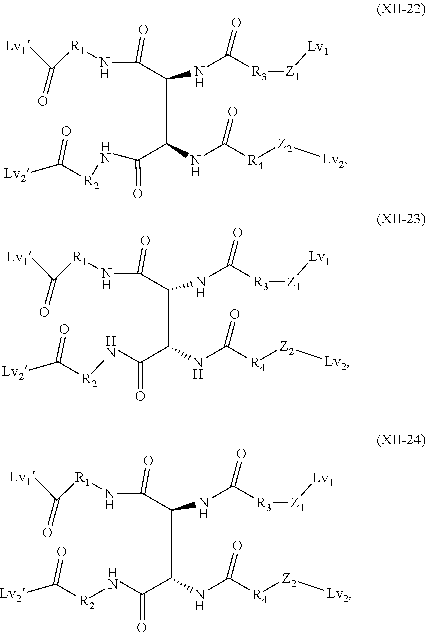
wherein “-----”, “ [symbol] ”, Q, X₁, X₂, Y₁, Y₂, R₁, R₂, R₃, R₄, R₅, R_{5′}, Z₁, Z₂, Lv₁, Lv₂, Lv₁′, Lv₂′, X¹ and X^{1′} are defined the same as in claim 48.

54. The conjugate compound according to claim 48, wherein Y₁, Y₂, Z₁ and Z₂ may link to pairs of thiols of the cell-binding agent through reducation from inter chain disulfide bonds of the cell-binding agent with dithiothreitol (DTT), dithioerythritol (DTE), L-glutathione (GSH), tris (2-carboxyethyl) phosphine (TCEP), 2-mercaptoethylamine (β-MEA), or/and beta mercaptoethanol (β-ME, 2-ME).

55. The conjugate compound according to claim 48, wherein the Drug₁ or Drug₂ is selected from the group consisting of:
(1) a chemotherapeutic agent selected from the group consisting of:
a) an alkylating agent: selected from the group consisting of nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: comprising busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine;
b) a plant alkaloid: selected from the group consisting of Vinca alkaloids: comprising vincristine, vinblastine, vindesine, vinorelbine, and navelbin; Taxoids: comprising paclitaxel, docetaxol and their analogs, Maytansinoids comprising DM1, DM2, DM3, DM4, DM5, DM6, DM7, maytansine, ansamitocins and their analogs, cryptophycins (including the group consisting of cryptophycin 1 and cryptophycin 8); epothilones, eleutherobin, discodermolide, bryostatins, dolostatins, auristatins, tubulysins, cephalostatins; pancratistatin; erbulins, a sarcodictyin; spongistatin;
c) a DNA Topoisomerase Inhibitor: selected from the group consisting of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d) an antimetabolite: selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (comprising mycophenolic acid, tiazofurin, ribavirin, EICAR); Ribonucleotide reductase Inhibitors: (comprising hydroxyurea, deferoxamine)]; [pyrimidine analogs: Uracil analogs: (comprising ancitabine, azacitidine, 6-azauridine, capecitabine (Xeloda), carmofur, cytarabine, dideoxyuridine, doxifluridine, enocitabine, 5-fluorouracil, floxuridine, ratitrexed (Tomudex)); Cytosine analogs: (comprising cytarabine, cytosine arabinoside, fludarabine); Purine analogs: (comprising azathioprine, fludarabine, mercaptopurine, thiamiprine, thioguanine)]; folic acid replenisher, frolinic acid}; and Inhibitors of nicotinamide phosphoribosyltransferase (NAMPT);
e) a hormonal therapy: selected from the group consisting of {Receptor antagonists: [Anti-estrogen: (comprising megestrol, raloxifene, tamoxifen); LHRH agonists: (comprising goscrclin, leuprolide acetate); Anti-androgens: (comprising bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (comprising CB 1093, EB 1089 KH 1060, cholecalciferol, ergocalciferol); Photodynamic therapies: (comprising verteporfin, phthalocyanine, photosensitizer Pc4, demethoxyhypocrellin A); Cytokines: (comprising Interferon-alpha, Interferon-gamma, tumor necrosis factor (TNFs), human proteins containing a TNF domain)]};
f) a kinase inhibitor selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib;
g) a poly (ADP-ribose) polymerase (PARP) inhibitors selected from the group consisting of olaparib, niraparib, iniparib, talazoparib, veliparib, CEP 9722 (Cephalon's), E7016 (Eisai's), BGB-290 (BeiGene's), or 3-aminobenzamide;
h) an antibiotic, selected from the group consisting of an enediyne antibiotic (selected from the group consisting of calicheamicin, calicheamicin γ1, δ1, α1 or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
i) a polyketide (acetogenin), bullatacin and bullatacinone; gemcitabine, epoxomicins andcarfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins and 1-methyl-4-phenylpyridinium ion, Cell cycle inhibitors (selected from staurosporine), Actinomycins (comprising Actinomycin D, dactinomycin), amanitins, Bleomycins (comprising bleomycin A2, bleomycin B2, peplomycin), Anthracyclines (comprising daunorubicin, doxorubicin (adriamycin), idarubicin, epirubicin, pirarubicin, zorubicin, mtoxantrone, MDR inhibitors or verapamil, Ca²⁺ ATPase inhibitors or thapsigargin, Histone deacetylase inhibitors ((comprising Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, selected from the group consisting of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (including the group consisting ofT-2 toxin, verrucarin A, roridin A and anguidine); urethane, siRNA, antisense drugs;
(2) an anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus;
(3) an anti-infectious disease agents comprising:
a) Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b) Amphenicols: azidamfenicol, chloramphenicol, florfenicol, thiamphenicol;
c) Ansamycins: geldanamycin, herbimycin;
d) Carbapenems: biapenem, doripenem, ertapenem, imipenem/cilastatin, meropenem, panipenem;
e) Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef);
f) Glycopeptides: bleomycin, vancomycin (oritavancin, telavancin), teicoplanin (dalbavancin), ramoplanin;
g) Glycylcyclines: tigecycline;
h) p-Lactamase inhibitors: penam (sulbactam, tazobactam), clavam (clavulanic acid);
i) Lincosamides: clindamycin, lincomycin;
j) Lipopeptides: daptomycin, A54145, calcium-dependent antibiotics (CDA);
k) Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l) Monobactams: aztreonam, tigemonam;
m) Oxazolidinones: linezolid;
n) Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o) Polypeptides: bacitracin, colistin, polymyxin B;
p) Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q) Streptogramins: pristinamycin, quinupristin/dalfopristin;
r) Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s) Steroid antibacterials: selected from fusidic acid;
t) Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u) antibiotics: selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4) anti-viral drugs comprising:
a) Entry/fusion inhibitors: aplaviroc, maraviroc, vicriviroc, gp41 (enfuvirtide), PRO 140, CD4 (ibalizumab);
b) Integrase inhibitors: raltegravir, elvitegravir, globoidnan A;
c) Maturation inhibitors: bevirimat, vivecon;
d) Neuraminidase inhibitors: oseltamivir, zanamivir, peramivir;
e) Nucleosides &_nucleotides: abacavir, aciclovir, adefovir, amdoxovir, apricitabine, brivudine, cidofovir, clevudine, dexelvucitabine, didanosine (ddl), elvucitabine, emtricitabine (FTC), entecavir, famciclovir, fluorouracil (5-FU), 3′-fluoro-substituted 2′,3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of, β-1-thymidine and #-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f) non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g) protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h) anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib;
(5) a radioisotope that can be selected from the group consisting of (radionuclides)³H, ¹¹C, ¹⁴C, ¹⁸F, ³²P, ³⁵S, ⁶⁴Cu, ⁶⁸Ga, ⁸⁶Y ⁹⁹Tc, ¹¹¹In, ¹²³I, ¹²⁴I, ¹²⁵I, ¹³¹I, ¹³³Xe, ¹⁷⁷Lu, ²¹¹At, and ²¹³Bi;
(6) a chromophore molecule, which is capable of absorbing UV light, florescent light, IR light, near IR light, visual light; a class or subclass of xanthophores, erythrophores, iridophores, leucophores, melanophores, cyanophores, fluorophore molecules which are fluorescent chemical compounds reemitting light upon light, visual phototransduction molecules, photophore molecules, luminescence molecules, luciferin compounds; non-protein organic fluorophores, selected from: Xanthene derivatives (comprising fluorescein, rhodamine, Oregon green, eosin, and Texas red); Cyanine derivatives: (comprising cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (comprising dansyl and prodan derivatives); Coumarin derivatives; Oxadiazole derivatives (comprising pyridyloxazole, nitrobenzoxadiazole and benzoxadiazole); Anthracene derivatives (comprising anthraquinones, including DRAQ5, DRAQ7 and CyTRAK Orange); Pyrene derivatives (cascade blue); Oxazine derivatives (comprising Nile red, Nile blue, cresyl violet, oxazine 170). Acridine derivatives (comprising proflavin, acridine orange, acridine yellow); Arylmethine derivatives (comprising auramine, crystal violet, malachite green); Tetrapyrrole derivatives (comprising porphin, phthalocyanine, bilirubin); analogs and derivatives of the following fluorophore compounds comprising CF dye, DRAQ and CyTRAK probes, BODIPY, Alexa Fluor, DyLight Fluor, Atto and Tracy, FluoProbes, Abberior Dyes, DY and MegaStokes Dyes, Sulfo Cy dyes, HiLyte Fluor, Seta, SeTau and Square Dyes, Quasar and Cal Fluor dyes, SureLight Dyes (APC, RPEPerCP, Phycobilisomes), APC, APCXL, RPE, BPE, Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin (PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine, 7-AAD (7-aminoactinomycin D, CG-selective), Acridine Orange, Chromomycin A3, CyTRAK Orange (red excitation dark), DAPI, DRAQ5, DRAQ7, Ethidium Bromide, Hoechst33258, Hoechst33342, LDS 751, Mithramycin, Propidiumlodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1; A fluorophore compound: comprising DCFH (2′7′Dichorodihydro-fluorescein, oxidized form), DHR (Dihydrorhodamine 123, oxidized form, light catalyzes oxidation), Fluo-3 (AM ester. pH>6), Fluo-4 (AM ester. pH 7.2), Indo-1 (AM ester, low/high calcium (Ca2+)), SNARF (pH 6/9), Allophycocyanin (APC), AmCyan1 (tetramer, Clontech), AsRed2 (tetramer, Clontech), Azami Green (monomer), Azurite, B-phycoerythrin (BPE), Cerulean, CyPet, DsRed monomer (Clontech), DsRed2 (“RFP”), EBFP, EBFP2, ECFP, EGFP (weak dimer), Emerald (weak dimer), EYFP (weak dimer), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRed1, J-Red, Katusha, Kusabira Orange (monomer, MBL), mCFP, mCherry, mCitrine, Midoriishi Cyan (dimer, MBL), mKate (TagFP635, monomer), mKeima-Red (monomer), mKO, mOrange, mPlum, mRaspberry, mRFP1 (monomer), mStrawberry, mTFP1, mTurquoise2, P3 (phycobilisome complex), Peridinin Chlorophyll (PerCP), R-phycoerythrin (RPE), T-Sapphire, TagCFP (dimer), TagGFP (dimer), TagRFP (dimer), TagYFP (dimer), tdTomato (tandem dimer), Topaz, TurboFP602 (dimer), TurboFP635 (dimer), TurboGFP (dimer), TurboRFP (dimer), TurboYFP (dimer), Venus, Wild Type GFP, YPet, ZsGreen1 (tetramer), ZsYellow1 (tetramer) and their derivatives;
(7) cell-binding ligands or receptor agonists, which can be selected from: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting ofPyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRRs), selected from the group consisting of Toll-like receptors' (TLRs) ligands, C-type lectins and Nodlike Receptors' (NLRs) ligands; Calcitonin receptor agonists; integrin receptors' and their receptor subtypes' (selected from the group consisting of α_{V}β₁, α_{V}β₃, α_{V}βs, α_{V}β₆, α₆β₄, α_{V}β₁, α_{L}β₂, α_{IIb}β₃) agonists (selected from the group consisting of GRGDSPK, cyclo(RGDfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Nanobody (a derivative of VHH (camelid Ig)); Domain antibodies (dAb, a derivative of VH or VL domain); Bispecific T cell Engager (BiTE, a bispecific diabody); Dual Affinity ReTargeting (DART, a bispecific diabody); Tetravalent tandem antibodies (TandAb, a dimerized bispecific diabody); Anticalin (a derivative of Lipocalins); Adnectins (10th FN3 (Fibronectin)); Designed Ankyrin Repeat Proteins (DARPins); Avimers; EGF receptors and VEGF receptors' agonists;
(8) pharmaceutically acceptable salts, acids, derivatives, hydrate or hydrated salt; or a crystalline structure; or an optical isomer, racemate, diastereomer or enantiomer of any of the above drugs.

56. The conjugate compound according to claim 48, wherein the Drug, or Drug₂ is a chromophore molecule.

57. The conjugate compound according to claim 48, wherein the Drug, or Drug₂ is a polyalkylene glycol comprising poly(ethylene glycol) (PEGs), poly(propylene glycol), a copolymer of ethylene oxide or propylene oxide, or their analogs.

58. The conjugate compound according to claim 48, wherein the Drug, or Drug₂ is a cell-binding ligand, a cell receptor agonist, or a cell receptor binding molecule.

59. The conjugate compound of claim 48, wherein the Drug, or Drug₂ is selected from the group consisting of tubulysins, calicheamicins, auristatins, maytansinoids, CC-1065 analogs, daunorubicin and doxorubicin compounds, taxanoids (taxanes), cryptophycins, epothilones, benzodiazepine dimers (comprising pyrrolobenzodiazepine dimers (PBD), tomaymycin dimers, anthramycin dimers, indolinobenzodiazepine dimers, imidazobenzothiadiazepine dimers, or oxazolidinobenzodiazepine dimers and their derivatives), calicheamicins and the enediyne antibiotics, actinomycins, amatoxins, amanitins, azaserines, bleomycins, epirubicins, tamoxifen, idarubicin, dolastatins/auristatins (comprising monomethyl auristatin E, MMAE, MMAF, auristatin PYE, auristatin TP, Auristatins 2-AQ, 6-AQ, EB (AEB), EFP (AEFP) and their analogs), duocarmycins, geldanamycins, methotrexates, thiotepa, vindesines, vincristines, hemiasterlins, nazumamides, microginins, radiosumins, alterobactins, microsclerodermins, theonellamides, esperamicins, erbulins, inhibitors of nicotinamide phosphoribosyltransferase (NAMPT), siRNA, miRNA, piRNA, nucleolytic enzymes, and/or pharmaceutically acceptable salts, acids, or/and their analogues, derivatives, hydrate or hydrated salt; or a crystalline structure; and an optical isomer, racemate, diastereomer or enantiomer of any of the above drugs thereof.

60. The conjugate compound according to claim 48, wherein the cell binding agent is selected from the group consisting of an antibody, a protein, probody, nanobody, a vitamin (including folate), peptides, a polymeric micelle, a liposome, a lipoprotein-based drug carrier, a nano-particle drug carrier, a dendrimer, and a molecule or a particle said above coating with cell-binding ligands, and a combination of said above thereof.

61. The conjugate compound according to claim 48, wherein the cell binding agent is selected from an antibody, an antibody-like protein, a full-length antibody (polyclonal antibody, monoclonal antibody, antibody dimer, antibody multimer), or multispecific antibody (selected from, bispecific antibody, trispecific antibody, or tetraspecific antibody); a single chain antibody, an antibody fragment that binds to the target cell, a monoclonal antibody, a single chain monoclonal antibody, or a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, diabody, triabody, tetrabody, miniantibody, a probody, a probody fragment, small immune proteins (SIP), a lymphokine, a hormone, a vitamin, a growth factor, a colony stimulating factor, a nutrient-transport molecule, large molecular weight proteins, nanoparticles or polymers modified with antibodies or large molecular weight proteins.

62. The conjugate compound according to claim 48, wherein the cell binding agent is capable of targeting against a tumor cell, a virus infected cell, a microorganism infected cell, a parasite infected cell, an autoimmune disease cell, an activated tumor cells, a myeloid cell, an activated T-cell, an affecting B cell, or a melanocyte, or any cells expressing any one of the following antigens or receptors: CD2, CD2R, CD3, CD3gd, CD3e, CD4, CD5, CD6, CD7, CD8, CD8a, CD8b, CD9, CD10, CD11a, CD11b, CD11c, CD12, CD12w, CD13, CD14, CD15, CD15s, CD15u, CD16, CD16a, CD16b, CD17, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD42a, CD42b, CD42c, CD42d, CD43, CD44, CD44R, CD45, CD45RA, CD45RB, CD45RO, CD46, CD47, CD47R, CD48, CD49a, CD49b, CD49c, CD49e, CD49f, CD50, CD51, CD52, CD53, CD54, CD55,CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD74, CD75, CD75s, CD76, CD77, CD78, CD79, CD79a, CD79b, CD80, CD81, CD82, CD83, CD84, CDw84, CD85, CD86, CD87, CD88, CD89, CD90, CD91, CD92, CDw92, CD93, CD94, CD95, CD96, CD97, CD98, CD99, CD99R, CD100, CD101, CD102, CD103, CD104, CD105, CD106, CD107, CD107a, CD107b, CD108, CD109, CD110, CD111, CD112, CD113, CDw113, CD114, CD115, CD116, CD117, CD118, CD119, CDw119, CD120a, CD120b, CD121a, CD121b, CDw121b, CD122, CD123, CDw123, CD124, CD125, CDw125, CD126, CD127, CD128, CDw128, CD129, CD130, CD131, CDw131, CD132, CD133, CD134, CD135, CD136, CDw136, CD137, CDw137, CD138, CD139, CD140a, CD140b, CD141, CD142, CD143, CD144, CD145, CDw145, CD146, CD147, CD148, CD149, CD150, CD151, CD152, CD153, CD154, CD155, CD156a, CD156b, CDw156c, CD157, CD158a, CD158b, CD159a, CD159b, CD159c, CD160, CD161, CD162, CD162R, CD163, CD164, CD165, CD166, CD167, CD167a, CD168, CD169, CD170, CD171, CD172a, CD172b, CD172g, CD173, CD174, CD175, CD175s, CD176, CD177, CD178, CD179, CD180, CD181, CD182, CD183, CD184, CD185, CD186, CDw186, CD187, CD188, CD189, CD190, Cd191, CD192, CD193, CD194, CD195, CD196, CD197, CD198, CDw198, CD199, CDw199, CD200, CD200a, CD200b, CD201, CD202, CD202b, CD203, CD203c, CD204, CD205, CD206, CD207, CD208, CD209, CD210, CDw210, CD211, CD212, CD213a1, CD213a2, CD214, CD215, CD216, CDw217, CDw218a, CDw218b, CD219, CD220, CD221, CD222, CD223, CD224, CD225, CD226, CD227, CD228, CD229, CD230, CD231, CD232, CD233, CD234, CD235a, CD235ab, CD235b, CD236, CD236R, CD237, CD238, CD239, CD240, CD240CE, CD240D, CD241, CD242, CD243, CD244, CD245, CD246, CD247, CD248, CD249, CD250, CD251, CD252, CD253, CD254, CD256, CD257, CD258, CD259, CD260, CD261, CD262, CD263, CD264, CD265, CD266, CD267, CD268, CD269, CD270, CD271, CD272, CD273, CD274, CD275, CD276 (B7-H3), CD277, CD278, CD279, CD280, CD281, CD282, CD283, CD284, CD285, CD286, CD287, CD288, CD289, CD290, CD291, CD292, CDw293, CD294, CD295, CD296, CD297, CD298, CD299, CD300a, CD300c, CD300e, CD301, CD302, CD303, CD304, CD305, CD306, CD307, CD308, CD309, CD310, CD311, CD312, CD314, CD315, CD316, CD317, CD318, CD319, CD320, CD321, CD322, CD323, CD324, CDw325, CD326, CDw327, CDw328, CDw329, CD330, CD331, CD332, CD333, CD334, CD335, CD336, CD337, CDw338, CD339, 4-1 BB, 5AC, 5T4 (Trophoblast glycoprotein, TPBG, 5T4, Wnt-Activated Inhibitory Factor 1 or WAIF1), Adenocarcinomaantigen, AGS-5, AGS-22M6, Activin receptor-like kinase 1, AFP, AKAP-4, ALK, Alpha intergrin, Alpha v beta6, Amino-peptidase N, Amyloid beta, Androgen receptor, Angiopoietin 2, Angiopoietin 3, Annexin A1, Anthrax toxin-protective antigen, Anti-transferrin receptor, AOC3 (VAP-1), B7-H₃, Bacillus anthracisanthrax, BAFF (B-cell activating factor), B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11 (C—C motif chemokine 11), CCR4 (C—C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factorA, CRIPTO, FCSF1R (Colony stimulating factor 1 receptor, CD115), CSF2 (colony stimulating factor 2, Granulocyte-macrophage colony-stimulating factor (GM-CSF)), CTLA4 (cytotoxic T-lymphocyte associated protein 4), CTAA16.88 tumor antigen, CXCR4 (CD184),C—X—C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxintype-1, E. coli shiga toxintype-2, ED-B, EGFL7 (EGF-like domain-containing protein 7), EGFR, EGFRII, EGFRvIII, Endoglin (CD105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli,ETV6-AML, FAP (Fibroblast activation proteinalpha), FCGR1, alpha-Fetoprotein, Fibrin II, beta chain, Fibronectin extra domain-B, FOLR (folate receptor), Folate receptor alpha, Folate hydrolase, Fos-related antigen 1, F protein of respiratory syncytial virus, Frizzled receptor, Fucosyl GM1,GD2 ganglioside, G-28 (a cell surface antigen glyvolipid), GD3 idiotype, GloboH, Glypican 3, N-glycolylneuraminic acid, GM3, GMCSF receptor a-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influeza hemag-glutinin, IgE, IgE Fc region, IGHE, interleukins (IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (α4, α_{IIb}β₃, αvβ3, α4β7, α5β1, a6β4, a7β7, αIIβ3, α5β5, α_{V}β5), Interferon gamma-induced protein, ITGA2, ITGB2, KIR2D, LCK, Le, Legumain, Lewis-Y antigen, LFA-1(Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (mesothelin), MUC1(Mucin 1, cell surface associated (MUC1) orpolymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page4, PAP, Paratope of anti-(N-glycolylneuraminic acid), PAX3, PAX5, PCSK9, PDCD1 (PD-1, Programmed cell death protein 1,CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RANTES receptors (CCR1, CCR3, CCR5), RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R₁ (Tumor necrosis apoprosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TROP-2, TRP-2, Tyrosinase, VCAM-1 (CD106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

63. The conjugate compound according to claim 62, wherein the tumor cell is selected from the group consisting of lymphoma cells, myeloma cells, renal cells, breast cancer cells, prostate cancer cells, ovarian cancer cells, colorectal cancer cells, gastric cancer cells, squamous cancer cells, small-cell lung cancer cells, none small-cell lung cancer cells, testicular cancer cells, malignant cells, and cells that grow and divide at an unregulated, quickened pace to cause cancers.

64. The conjugate compound of claim 48, wherein the Drug, or Drug₂ is a chromophore molecule, the conjugate compound is selected from the group consisting of structures of Ac01, Ac02, Ac03, Ac04, Ac05, Ac06, and Ac07, Ac08, Ac09, Ac010, and Ac11 as following: 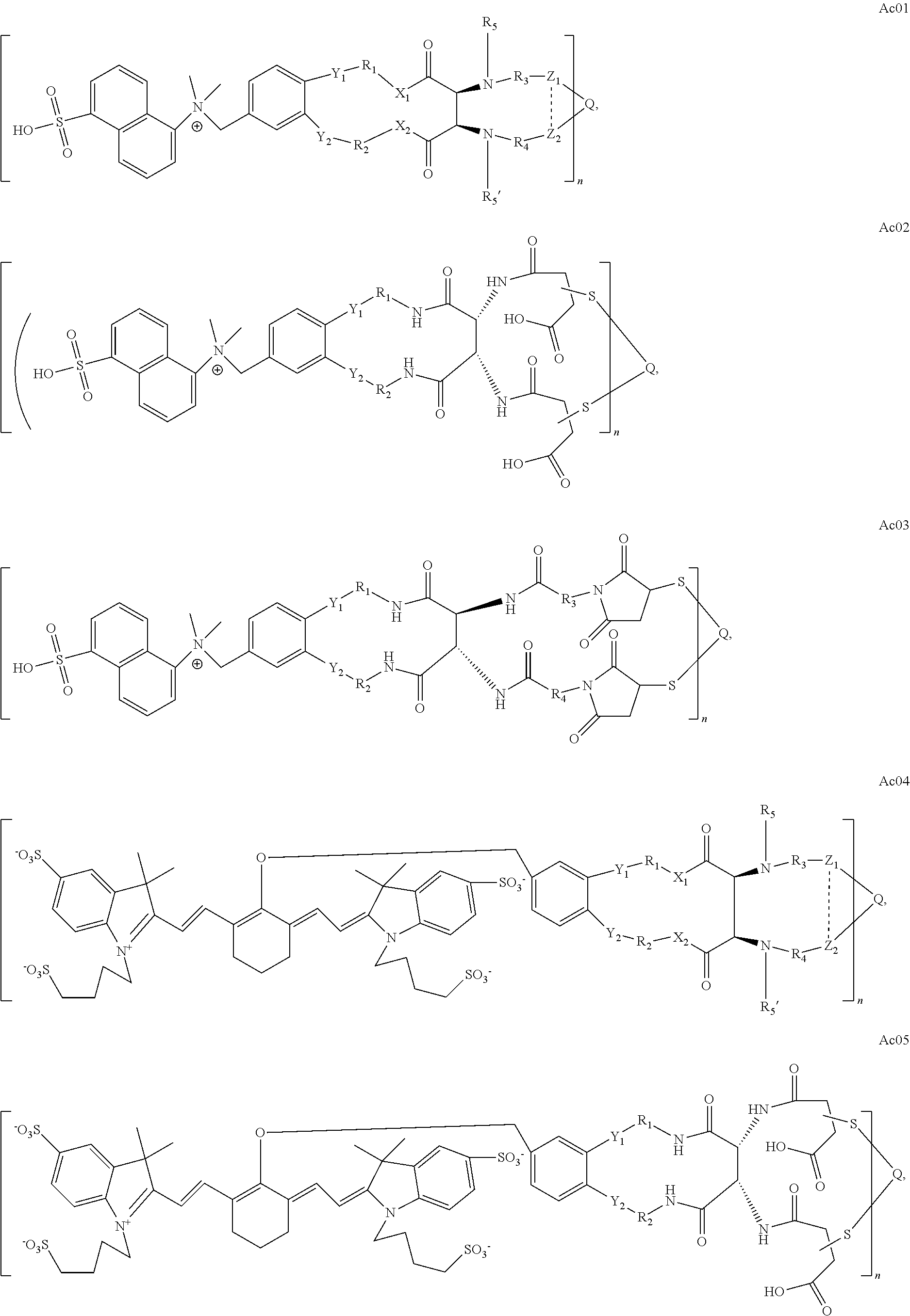 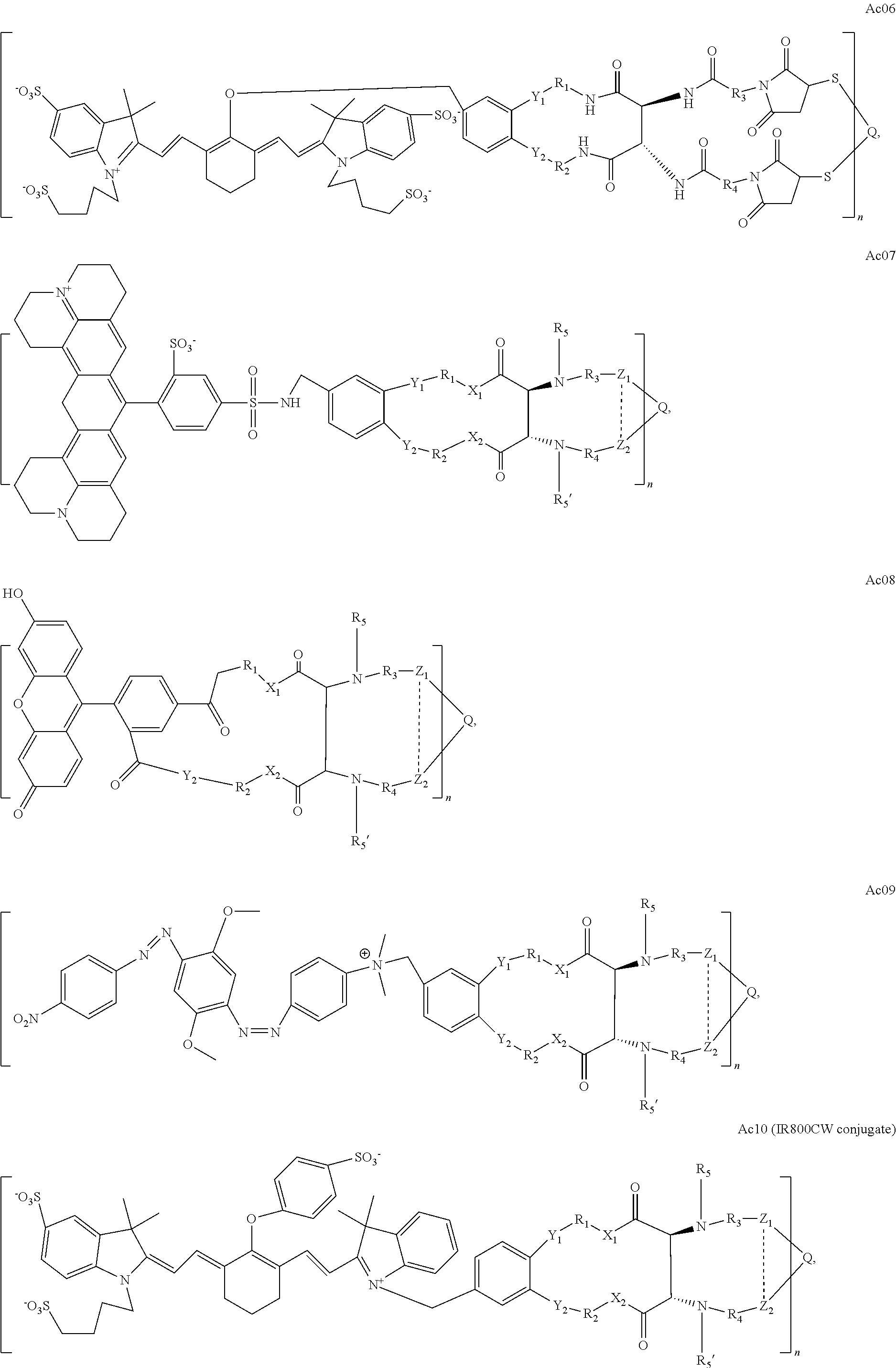 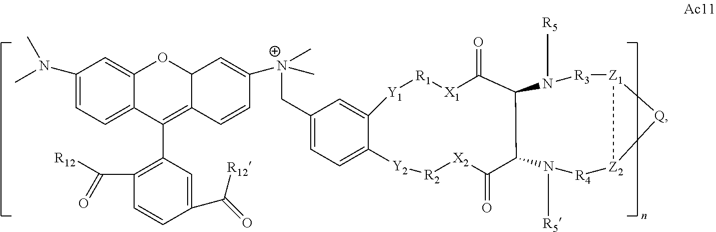
wherein “---”, Q, Y₁, Y₂, R₁, R₂, R₃, R₄, R₅, R_{5′}, Z₁, Z₂, and n are defined the same as in claim 48; R₁₂ and R₁₂′ are independently OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, O(CH₂CH₂O)ₚCH₂CH₂COOH, NH(CH₂CH₂O)ₚCH₂CH₂COOH, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, R₁—NHPO₃H₂, R₁—OPO₃H₂, O(CH₂CH₂O)ₚCH₂CH₂OPO₃H₂, NH(CH₂CH₂O)ₚCH₂CH₂NHPO₃H₂, OR₁—NHPO₃H₂, NH—R₁—NHPO₃H₂, NH—Ar—COOH, NH—Ar—NH₂, wherein p=0-5000, Aa is an aminoacid, (Aa)ₙ comprises the same or different, natural or unnatural amino acids, n=1-30.

65. The conjugate compound of claim 48, wherein the Drug, or Drug₂ is a tubulysin analog, the conjugate compound is selected from structures of T01, T02, T03, T04, T05, T06 T07, T08, T09, T10, T11, T12, T13, T14, T15, T16 T017, T18, T19, T20, T21, T22 and T23 as following: 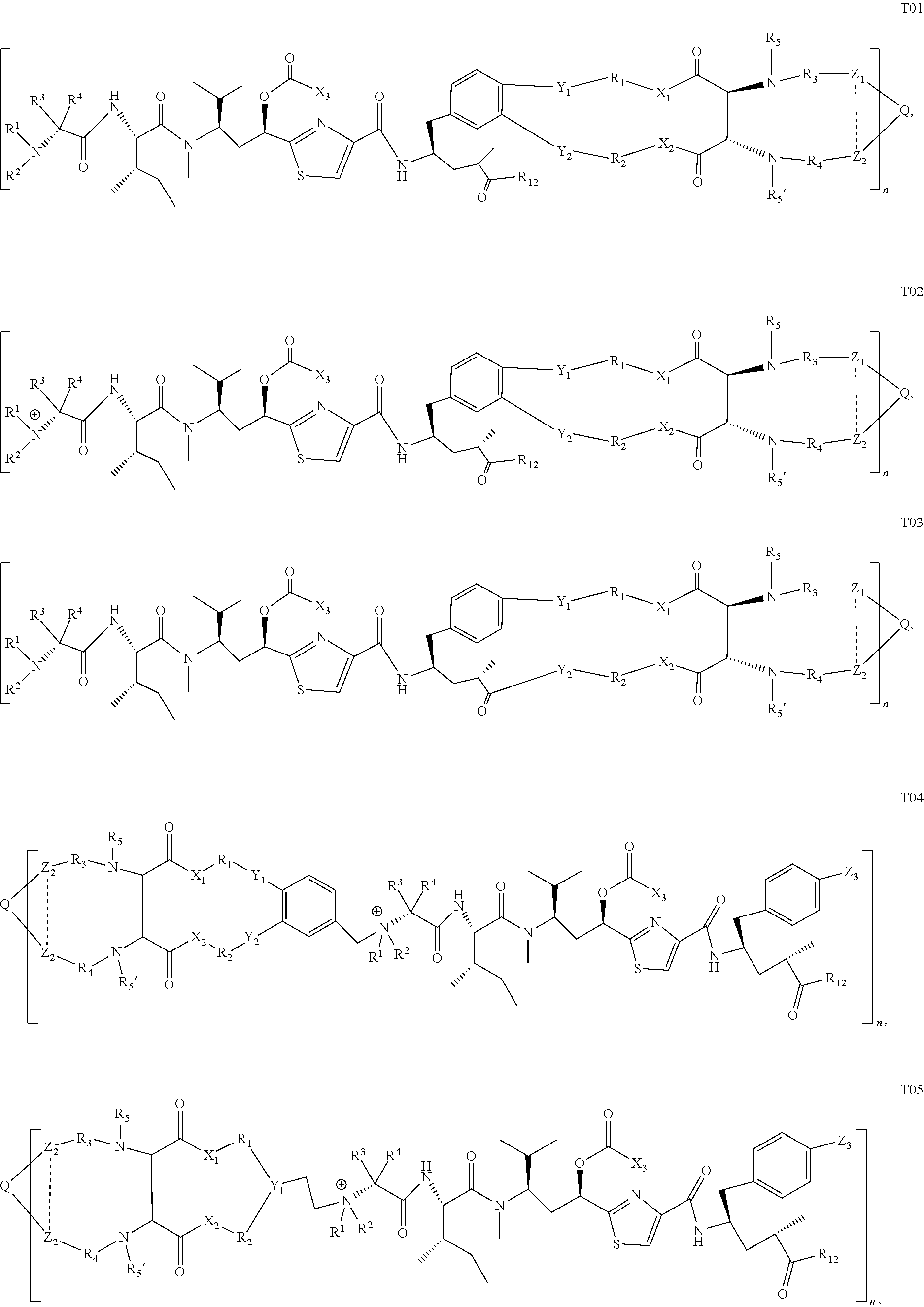 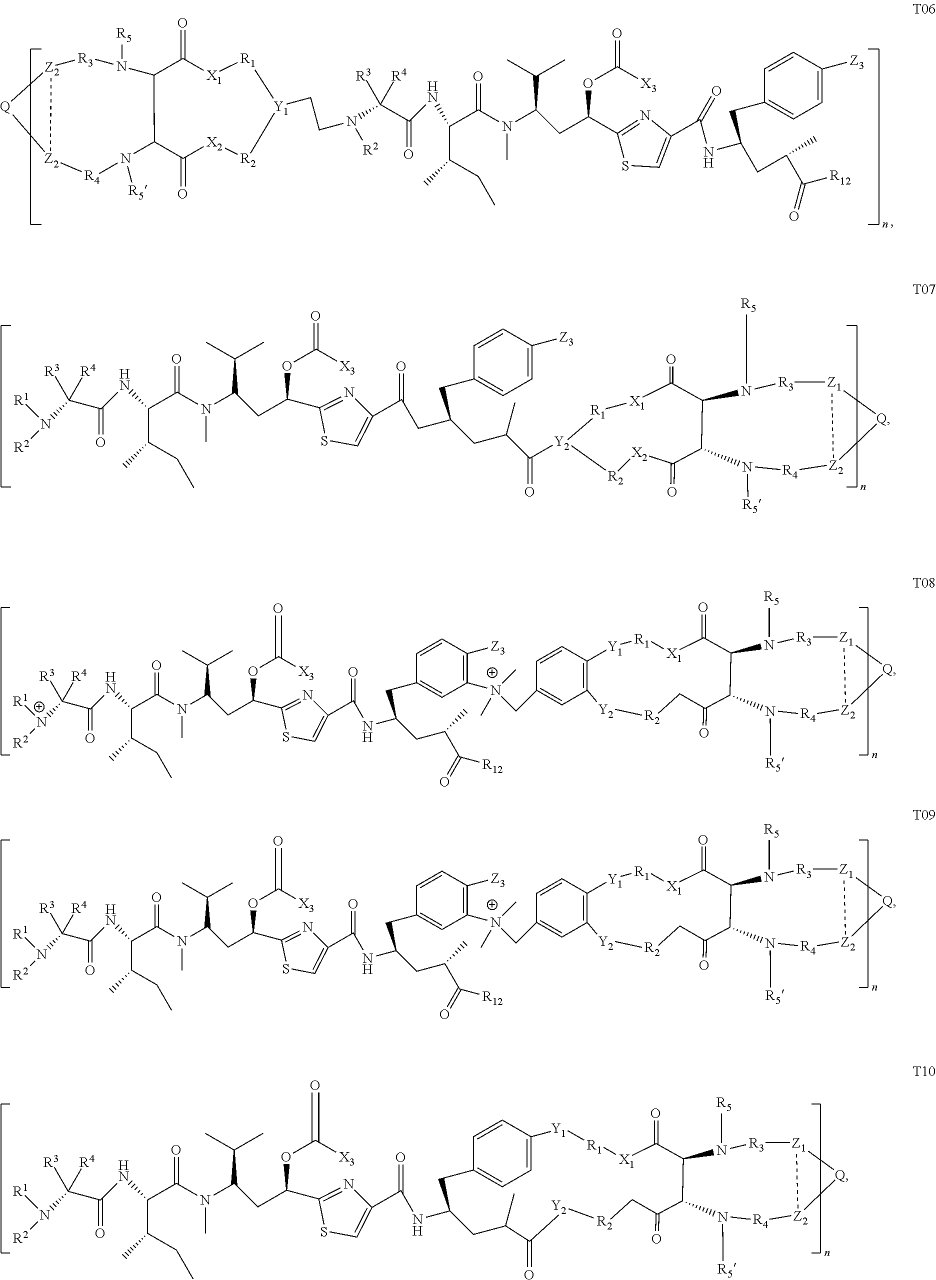 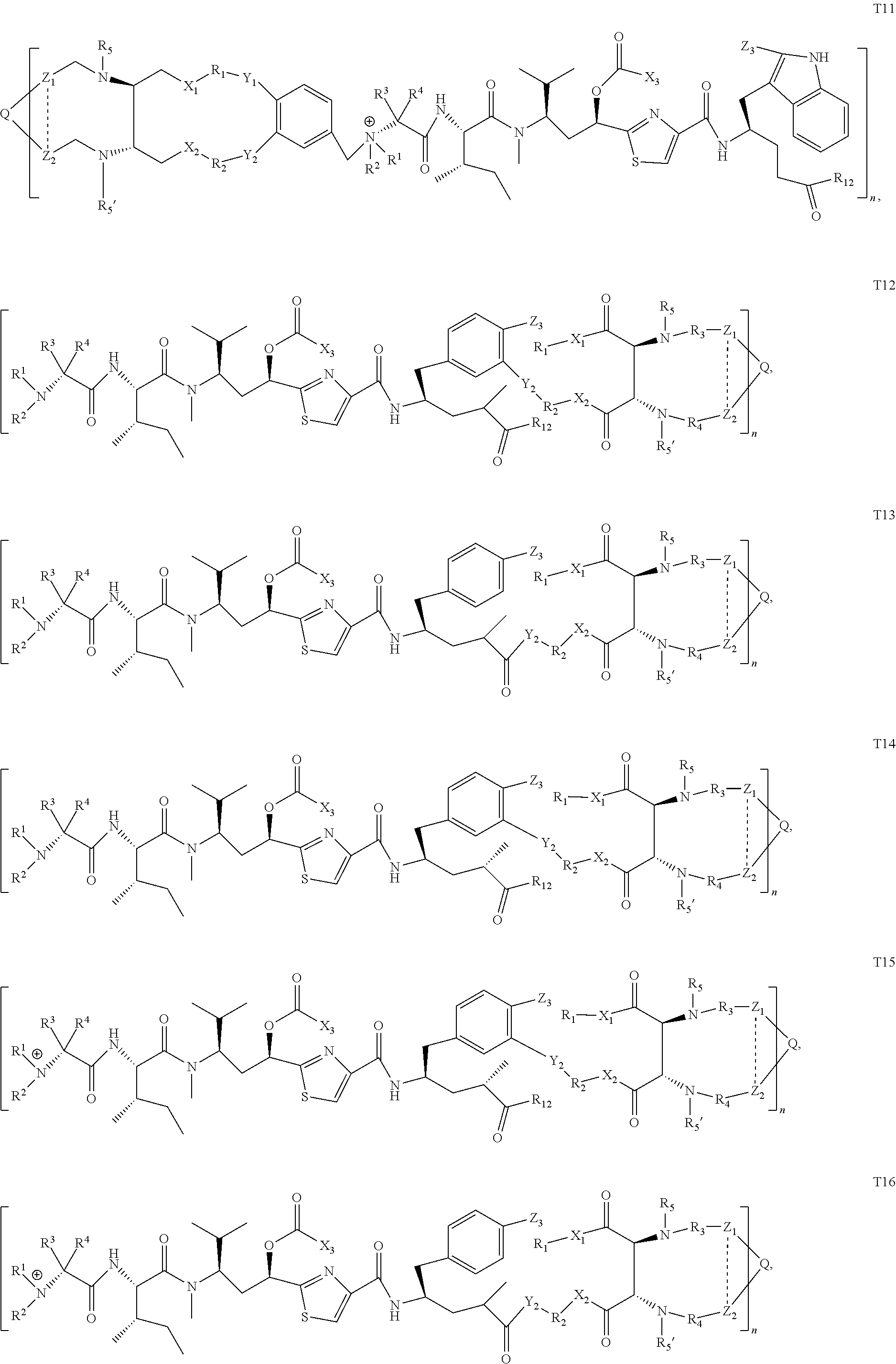 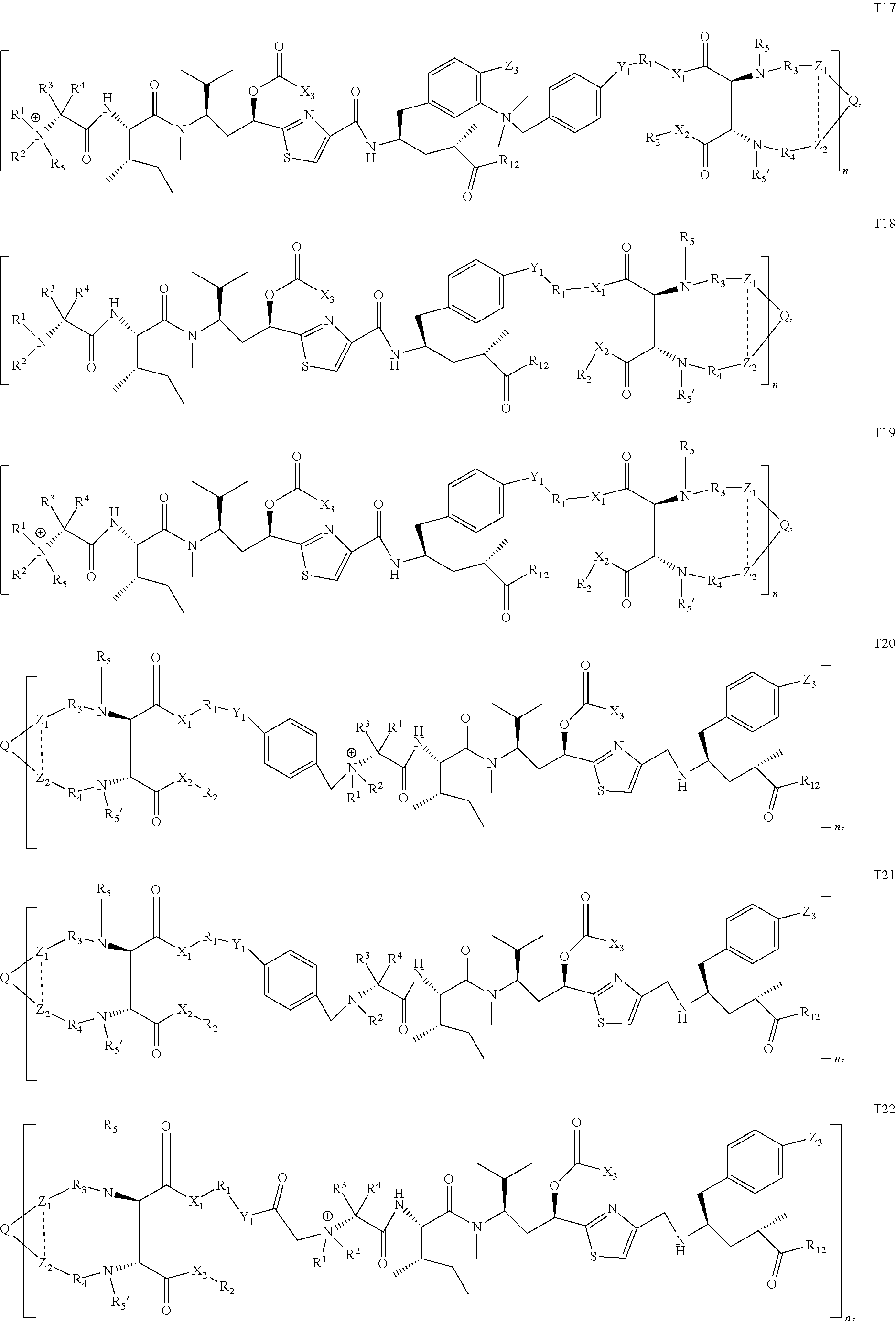 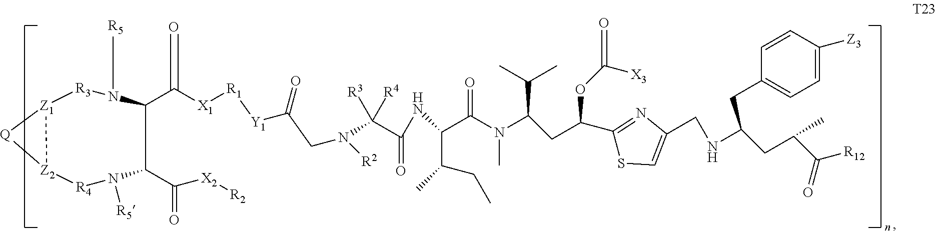
wherein “-----”, Q, X₁, X₂, R₁, R₂, R₃, R₄, R₅, R_{5′}, Aa, (Aa)ₙ, Z₁, Z₂, p, and n are defined the same as in claim 48; Y, and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is an antibody; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂CH₂NH₂, NH(CH₂CH₂S)ₚCH₂CH₂NH₂, NH(CH₂CH₂NH)ₚCH₂CH₂OH, NH(CH₂CH₂S)ₚCH₂CH₂OH, NH—R₁—NH₂, or NH(CH₂CH₂O)ₚCH₂CH₂NHPO₃H₂; R¹, R^{1′}, R², R³, R⁴ and R⁵ are independently H, C₁-C₈ lineal or branched alkyl, amide, or amines; C₂-C₈ aryl, alkenyl, alkynyl, heteroaryl, heteroalkyl, alkylcycloalkyl, ester, ether, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000; two Rs: R¹R², R²R³, R¹R³ or R³R⁴ can form 3-8 member cyclic ring of alkyl, aryl, heteroaryl, heteroalkyl, or alkylcycloalkyl group; X₃ is H, CH₃, CH₂CH₃, C₃H₇, or X₁′R₁′, wherein X₁′ is NH, N(CH₃), NHNH, O, or S; R₁′ is H or C₁-C₈ lineal or branched alkyl, aryl, heteroaryl, heteroalkyl, alkylcycloalkyl, or acyloxylamines; R₃′ is H or C₁-C₆ lineal or branched alkyl; Z₃ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃.

66. The conjugate compound of claim 48, wherein the Drug, or Drug₂ is a Calicheamicin analog, the conjugate compound is selected from structures of C01 and C02 as following: 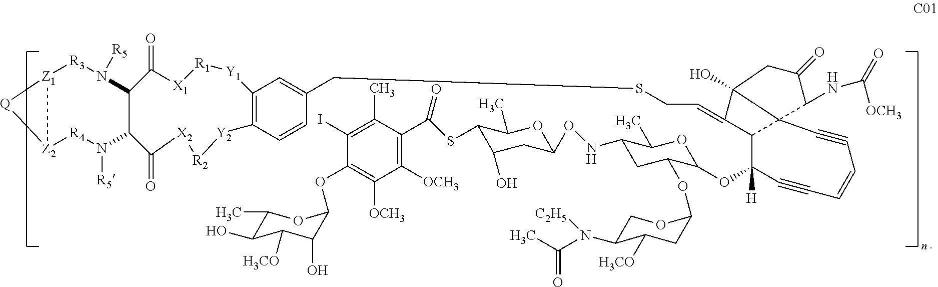 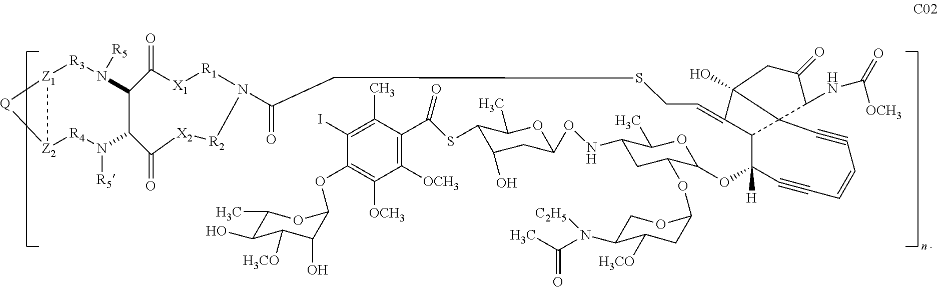
wherein “-----”, Q, X, X₂, Y₁, Y₂, R₁, R₂, R₃, R₄, R₅, R_{5′}, Z₁, Z₂, and n are defined the same as in claim 48; or X₁, X₂, Y, and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁.

67. The conjugate compound of claim 48, wherein the Drug₁ or Drug₂ is a Maytansinoid analog, the conjugate compound is selected from structures of the following My01, My02, My03, My04, My05, My06, My07, and My08: 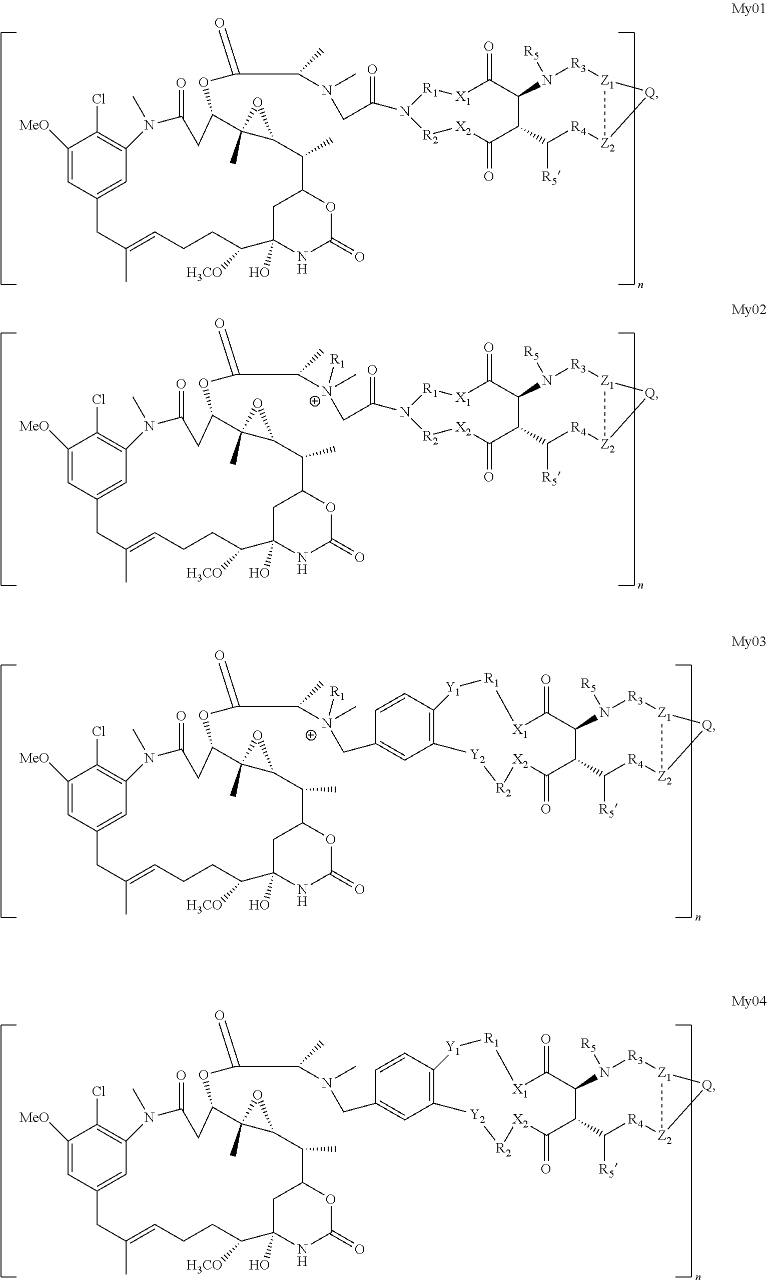 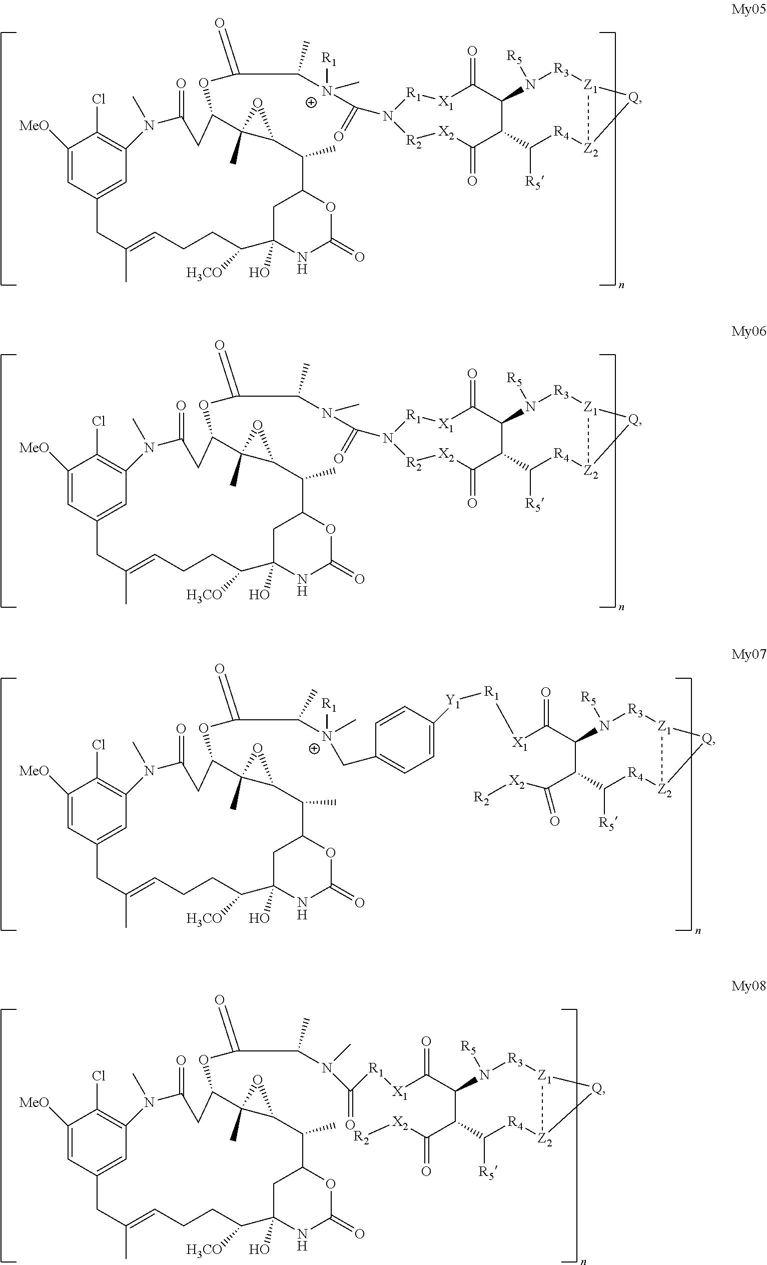
wherein “-----”, Q, X₁, X₂, Y₁, Y₂, R₁, R₂, R₃, R₄, R₅, R_{5′}, Z₁, Z₂, and n are defined the same as in claim 48; or X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁.

68. The conjugate compound of claim 48, wherein the Drug, or Drug₂ is a Taxane analog, the conjugate compound is selected from structures of Tx01, Tx02 and Tx03 as following: 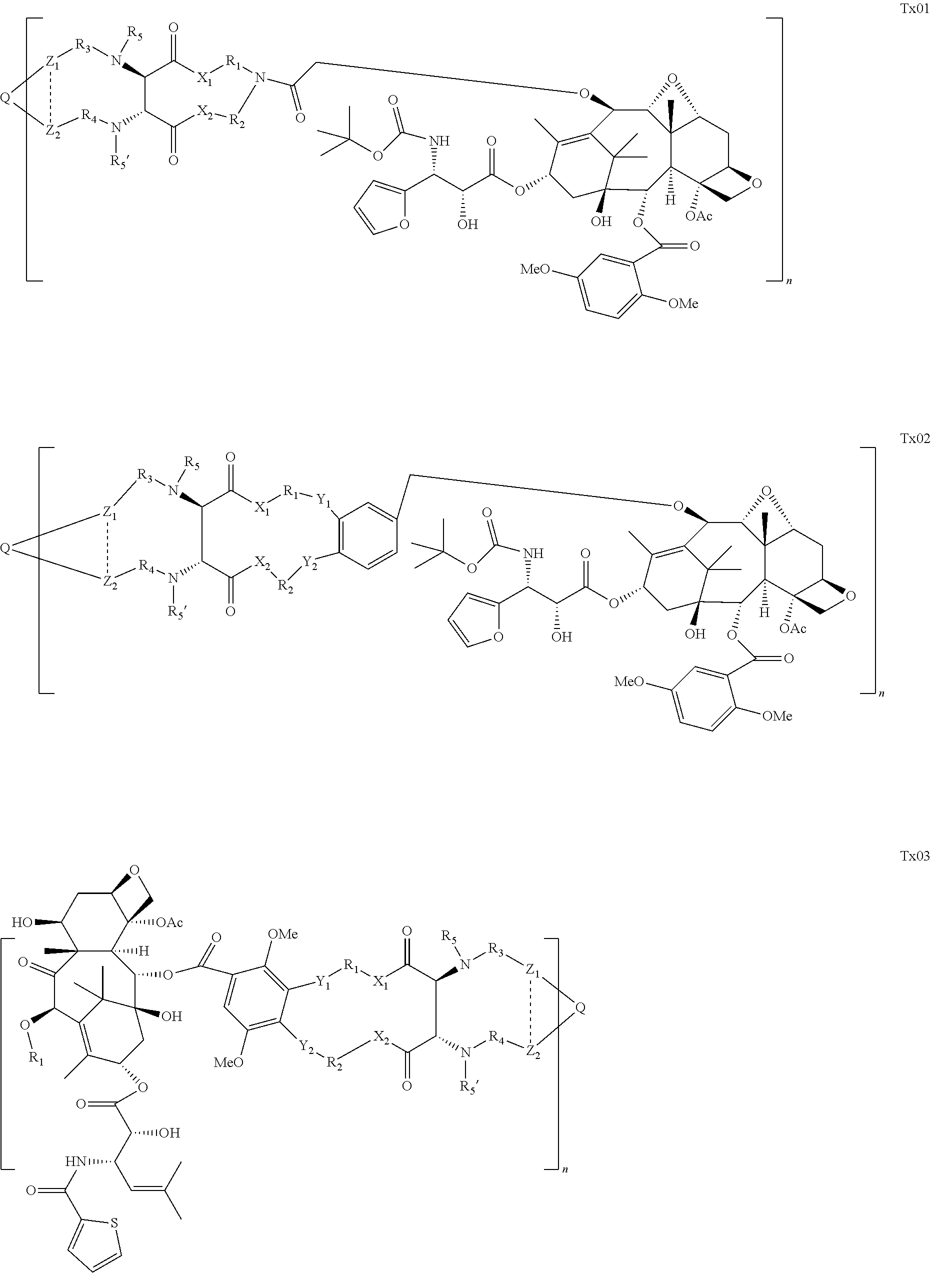
wherein “-----”, Q, X₁, X₂, Y₁, Y₂, R₁, R₂, R₃, R₄, R₅, R_{5′}, Z₁, Z₂, and n are defined the same as in claim 48; or X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁.

69. The conjugate compound of claim 48, wherein the Drug, or Drug₂ is a CC-1065 analogue and/or duocarmycin analog, the conjugate compound is selected from structures of CC01, CC02, CC03, CC04, CC05, CC06 and CC07 as following: 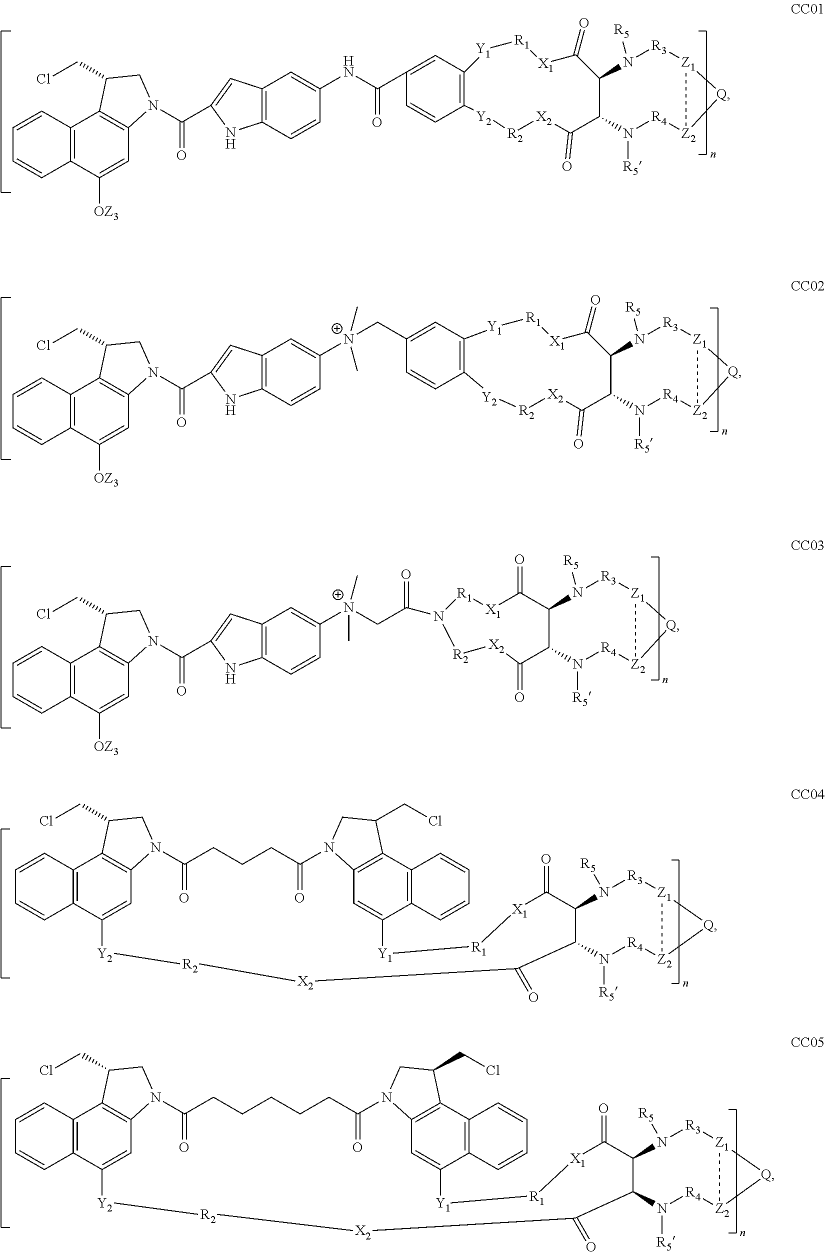 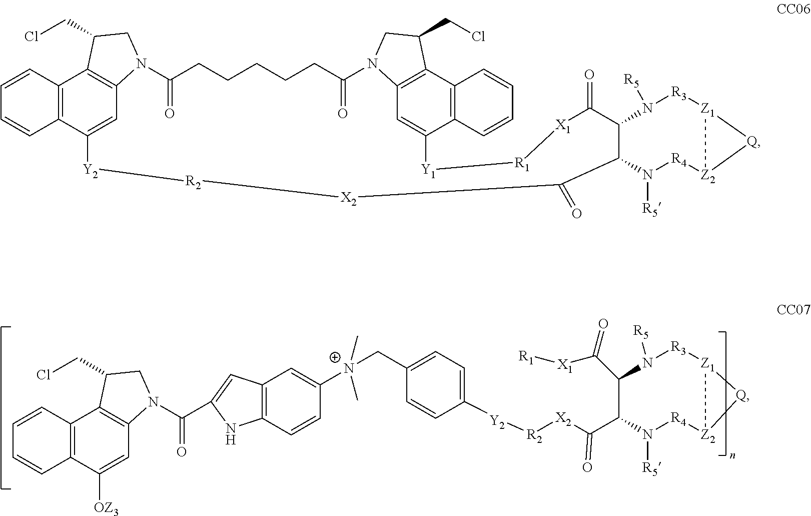
wherein “-----”, Q, X₁, X₂, Y₁, Y₂, R₁, R₂, R₃, R₄, R₅, R_{5′}, Z₁, Z₂, and n are defined the same as in claim 48; or X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; Q is monoclonal antibody; Z₃ is H, PO(OM₁)(OM₂), SO₃M₁, CH₂PO(OM,)(OM₂), CH₃N(CH₂CH₂)₂NC(O)—, O(CH₂CH₂)₂NC(O)—, R₁, or glycoside.

70. The conjugate compound of claim 48, wherein the Drug₁ or Drug₂ is a Daunorubicin or Doxorubicin analogue, the conjugate compound is selected from structures of Da01, Da02, Da03, Da04, Da05, Da06, Da07, Da08, Da09, Da10, and Da11 as following: 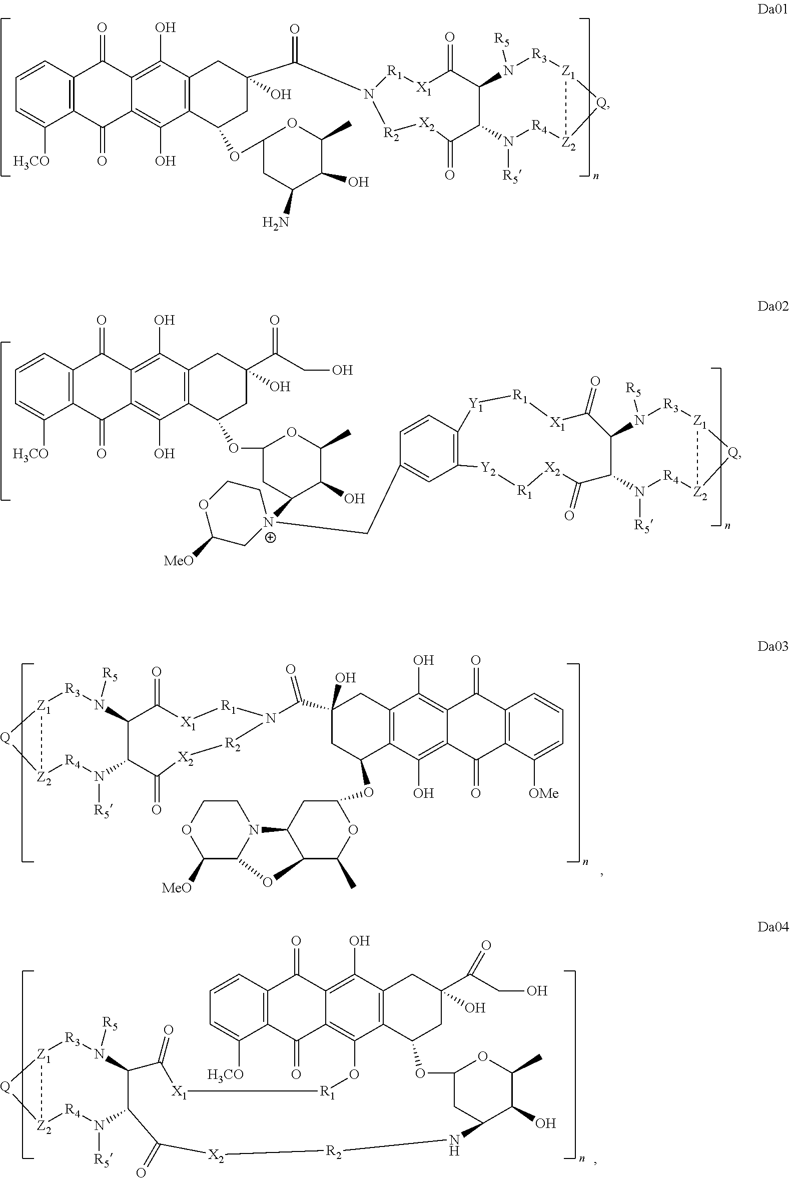 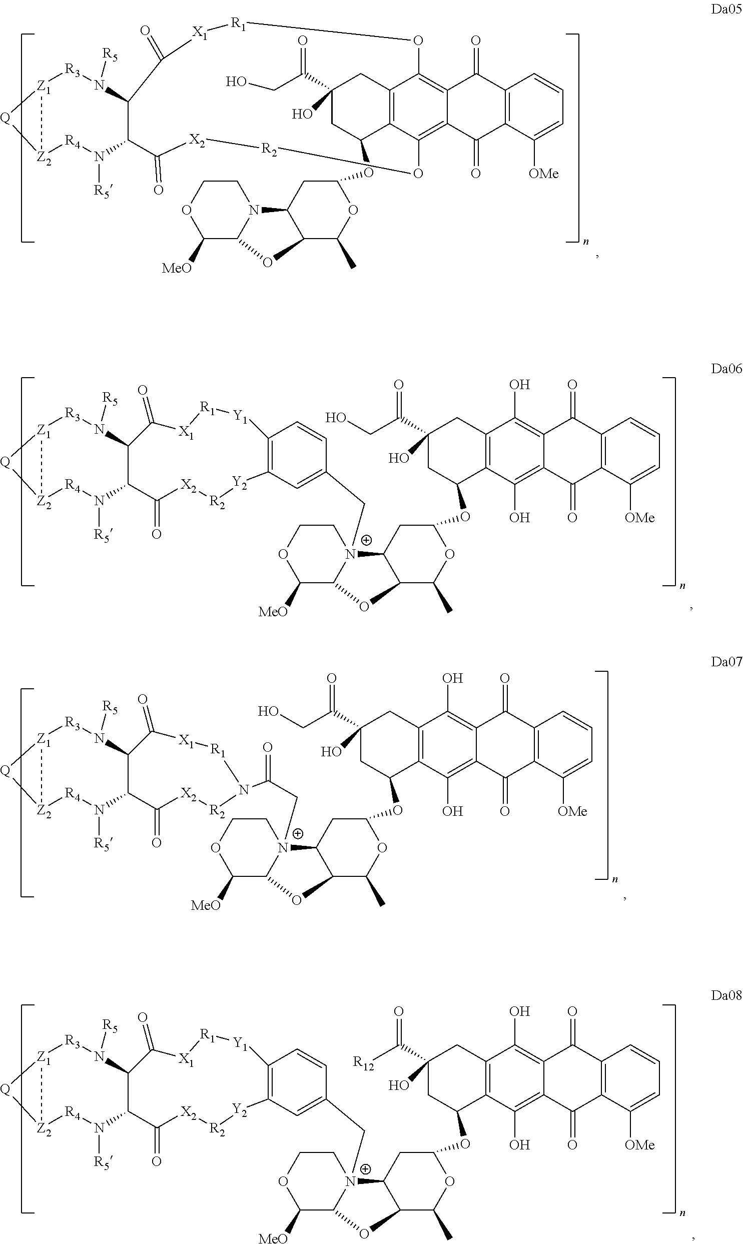 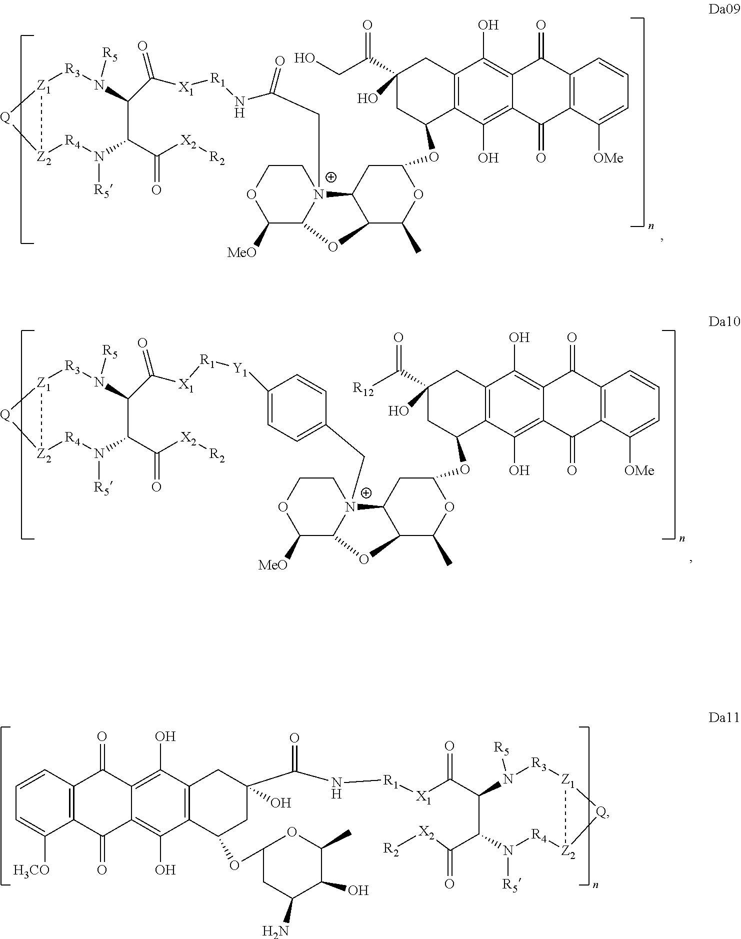
wherein “-----”, Q, X₁, X₂, Y₁, Y₂, R₁, R₂, R₃, R₄, R₅, R_{5′}, Z₁, Z₂, Aa, (Aa)ₙ, p, and n are defined the same as in claim 48; or X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R₁₂ is OH, NH₂, NHR₁, NHNH₂, NHNHCOOH, O—R₁—COOH, NH—R₁—COOH, NH(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NH—SO₃H, R₁—NHSO₃H, NH—R₁—NHSO₃H, O(CH₂CH₂O)ₚCH₂—CH₂NHPO₃H₂, NH(CH₂CH₂O)ₚCH₂—CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂.

71. The conjugate compound of claim 48, wherein the Drug, or Drug₂ is an Auristatin or dolastatin analogue, the conjugate compound is selected from structures of Au01, Au02, Au03, Au04, Au05, Au06, Au07, Au08, Au09, Au10, Au11, Au12, Au13, Au14, Au15, Au16, Au17, Au18, Au19, Au20, Au21, Au22, Au23, Au24, Au25, Au26, and Au27 as following: 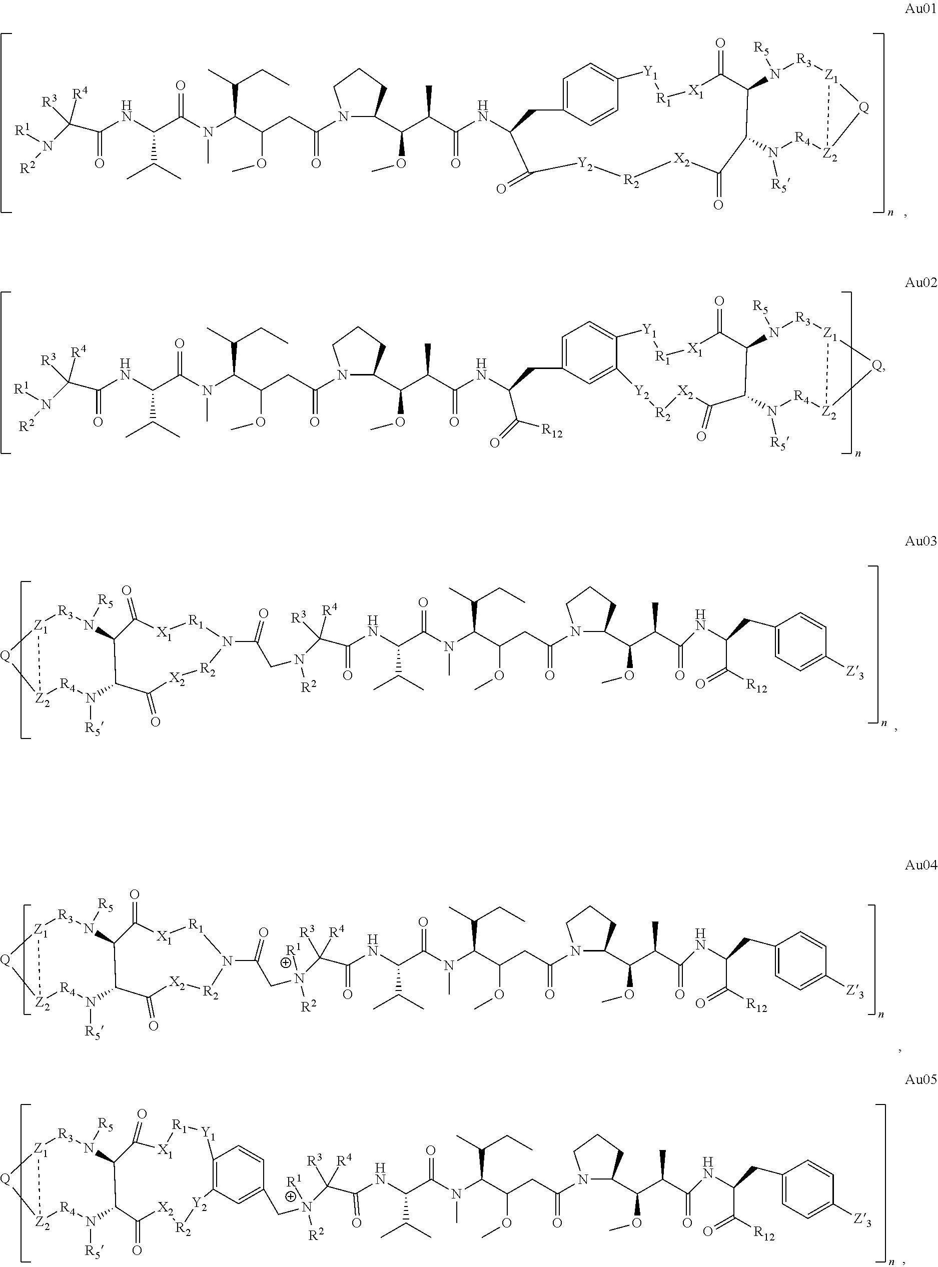 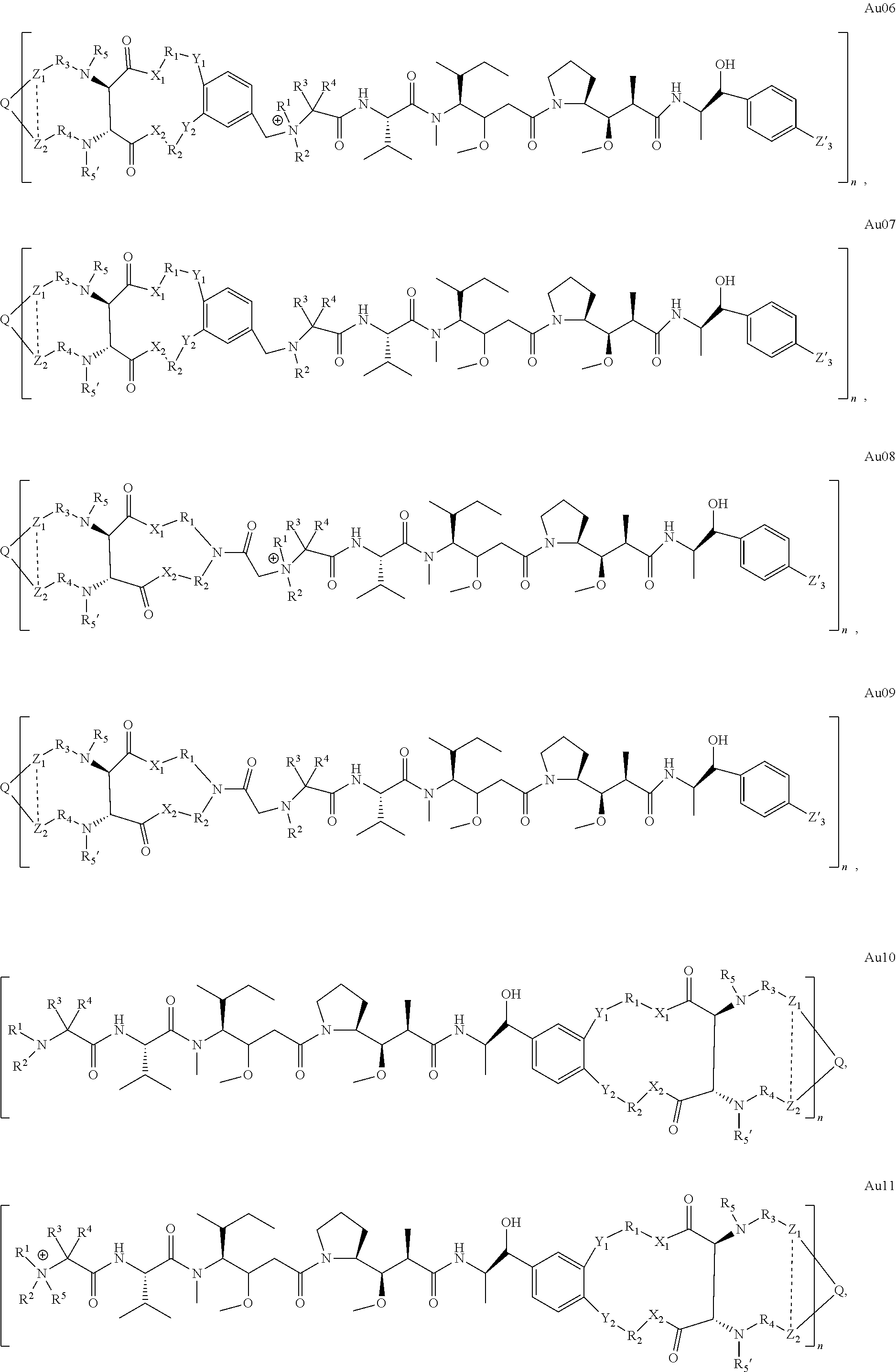 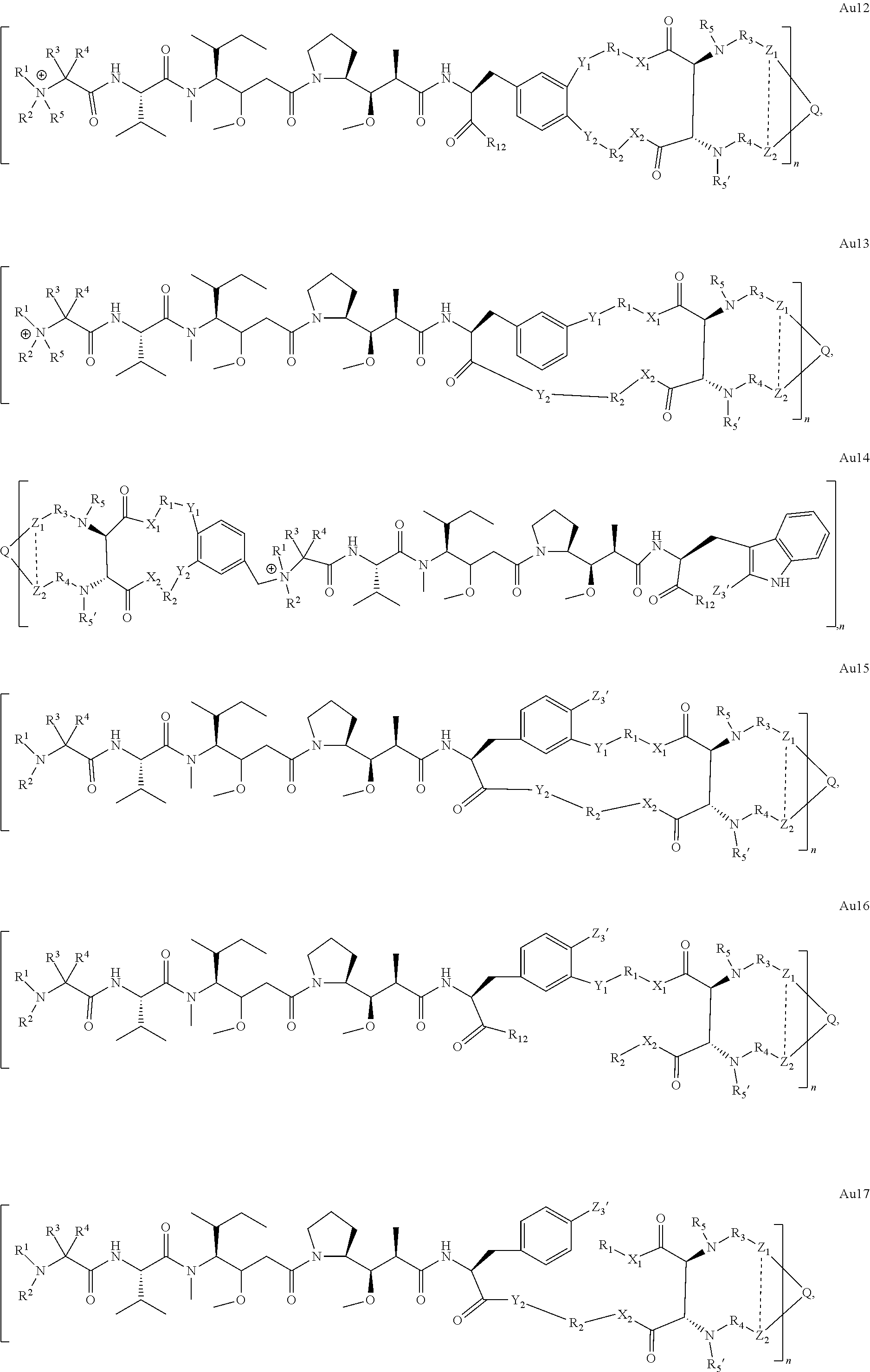 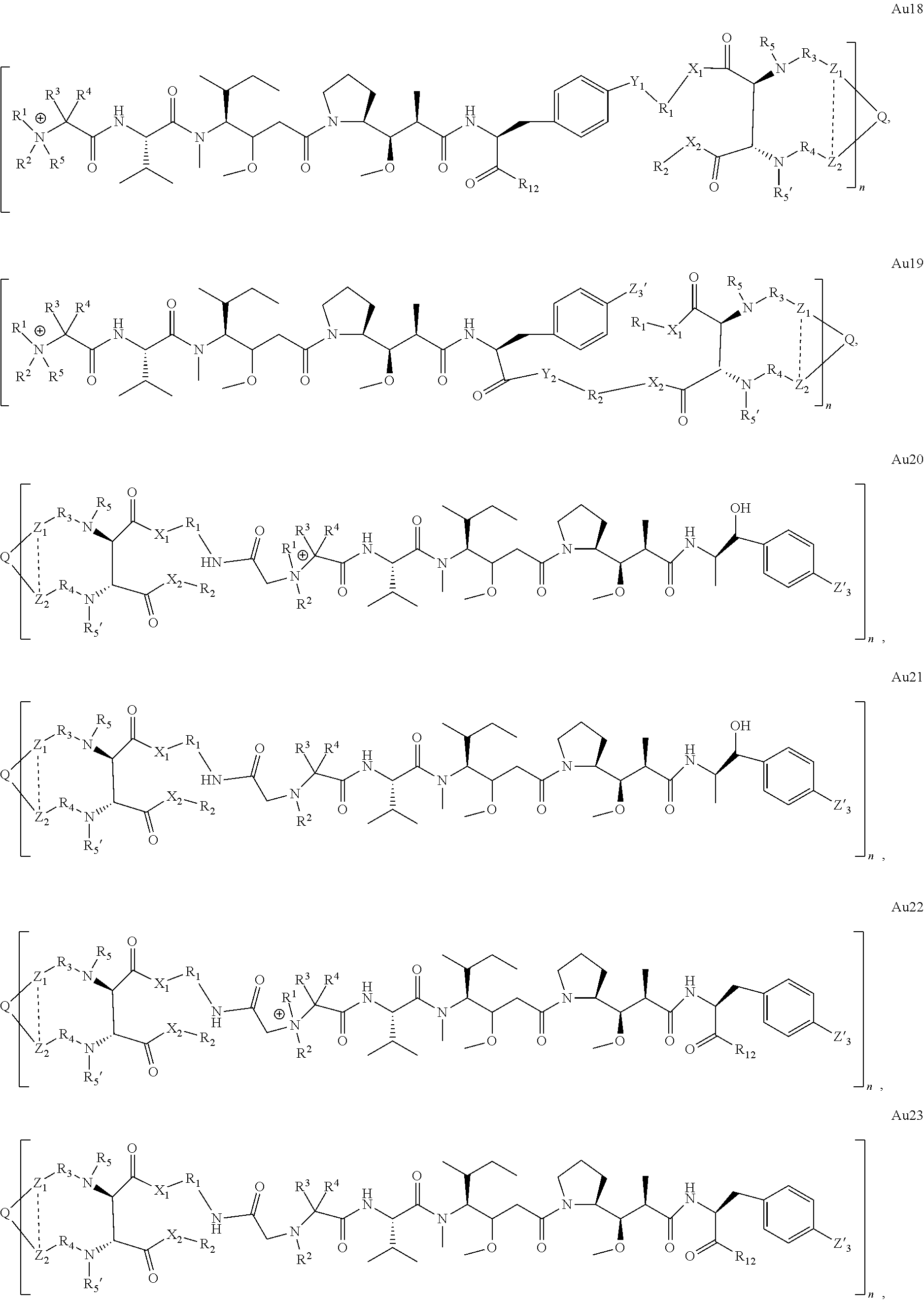 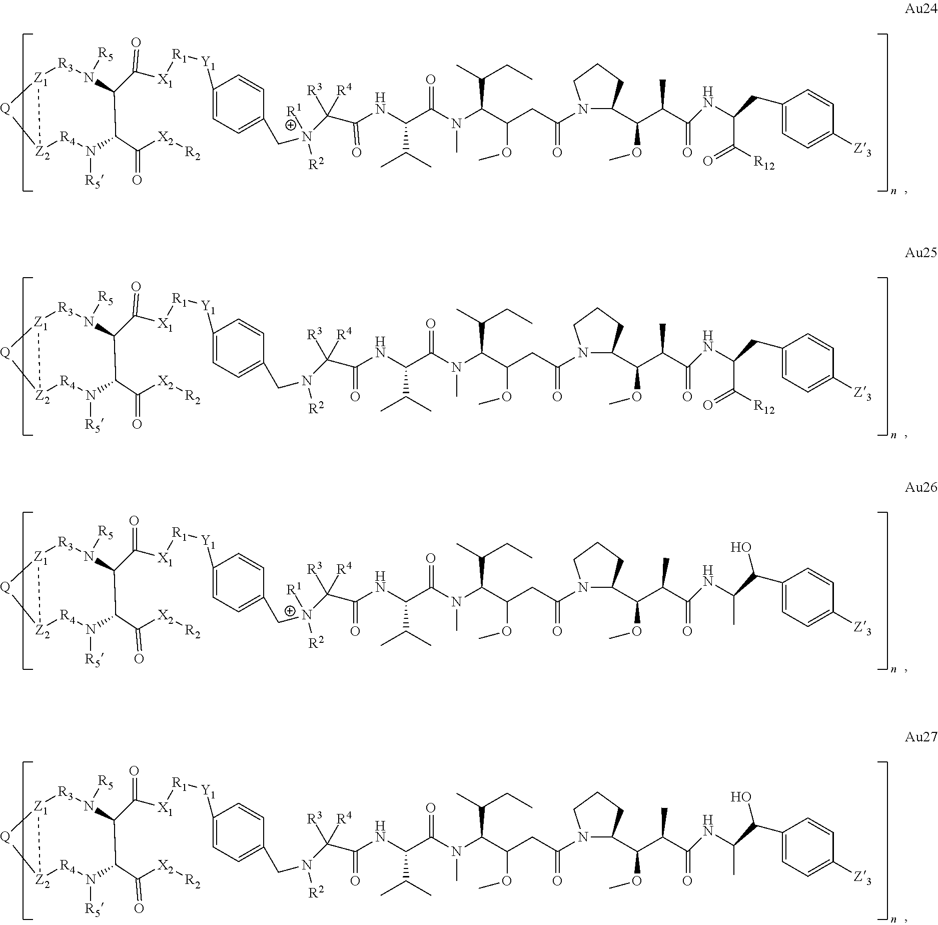
wherein “-----”, Q, X₁, X₂, Y₁, Y₂, R₁, R₂, R₃, R₄, R₅, R_{5′}, Z₁, Z₂, Aa, (Aa)ₙ, p and n are defined the same as in claim 48; or X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂; R¹, R², R³, R⁴ and R⁵ are independently H; C₁-C₈ lineal or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000; two Rs: R¹R₂, R²R₃, R¹R³ or R³R⁴ can form 3-8 member cyclic ring of alkyl, aryl, heteroaryl, heteroalkyl, or alkylcycloalkyl group; X₃ is H, CH₃ or X₁′R₁′, wherein X₁′ is NH, N(CH₃), NHNH, O, or S, and R₁′ is H or C₁—C lineal or branched alkyl, aryl, heteroaryl, heteroalkyl, alkylcycloalkyl, acyloxylamines; R₃′ is H or C₁-C₈ lineal or branched alkyl; Z₃′ is H, COOR₁, NH₂, NHR₁, OR₁, CONHR₁,NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, or NR₁R₂R₃.

72. The conjugate compound of claim 48, wherein the Drug₁ or Drug₂ is a dimer of benzodiazepine analogues, the conjugate compound is selected from structures of PB01, PB02, PB03, PB04, PB05, PB06, PB07, PB08, PB09, PB10, PB11, PB12, PB13, PB14, PB15, PB16, PB17, PB18, PB19, PB20, PB21, PB22, PB23, PB24, PB25, PB26, PB27, PB28, PB29, PB30, PB31 and PB32: 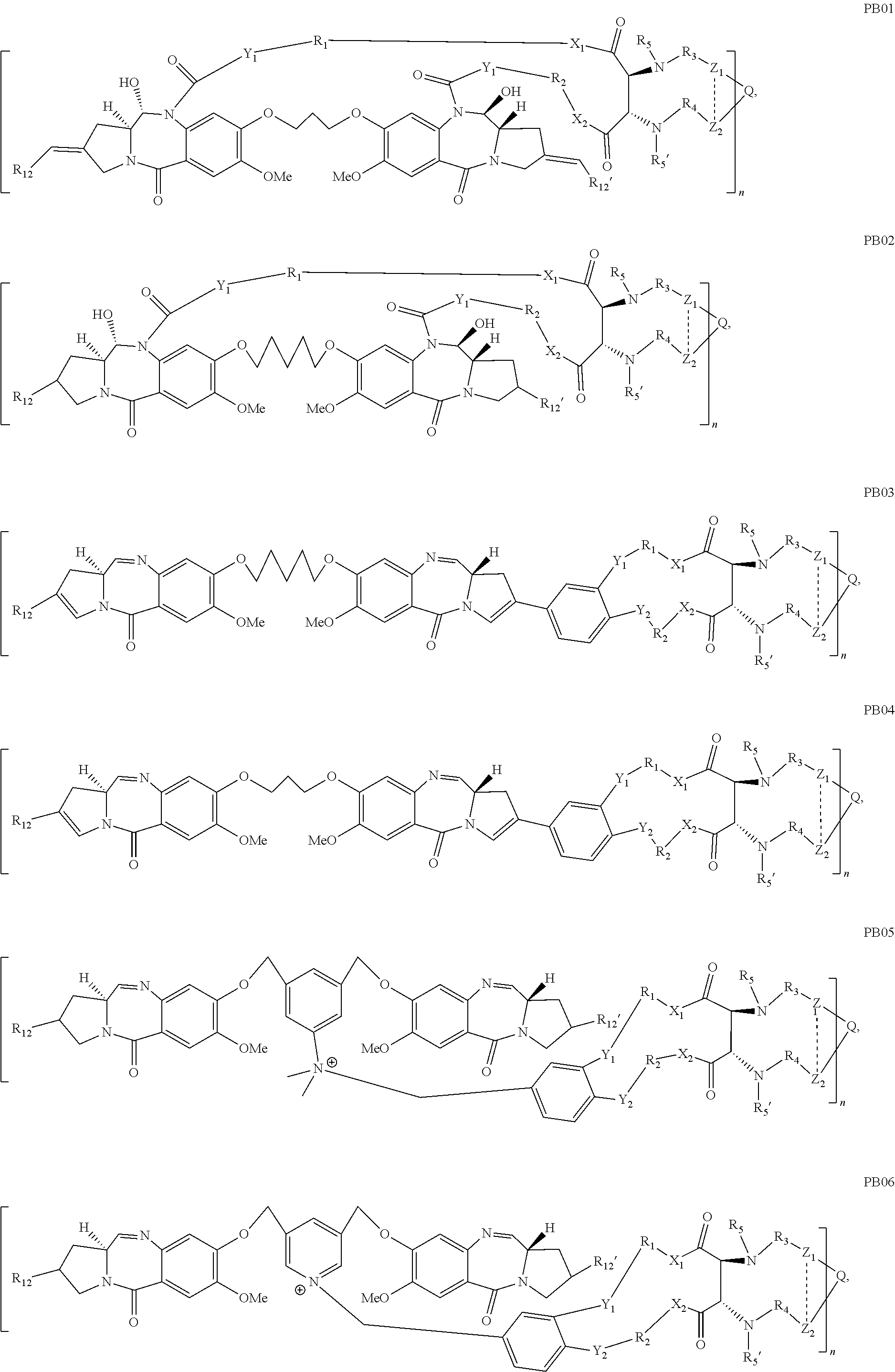 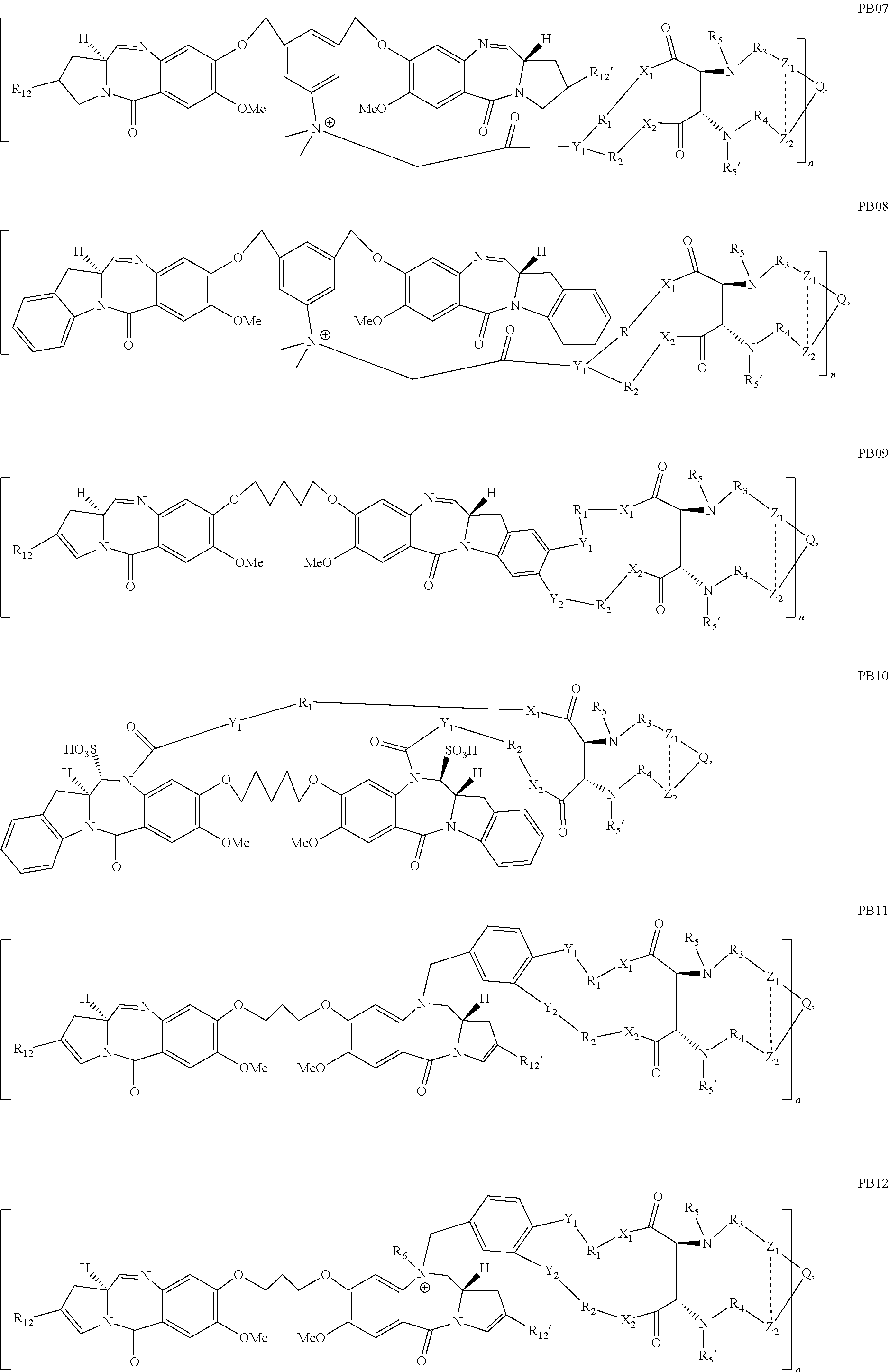 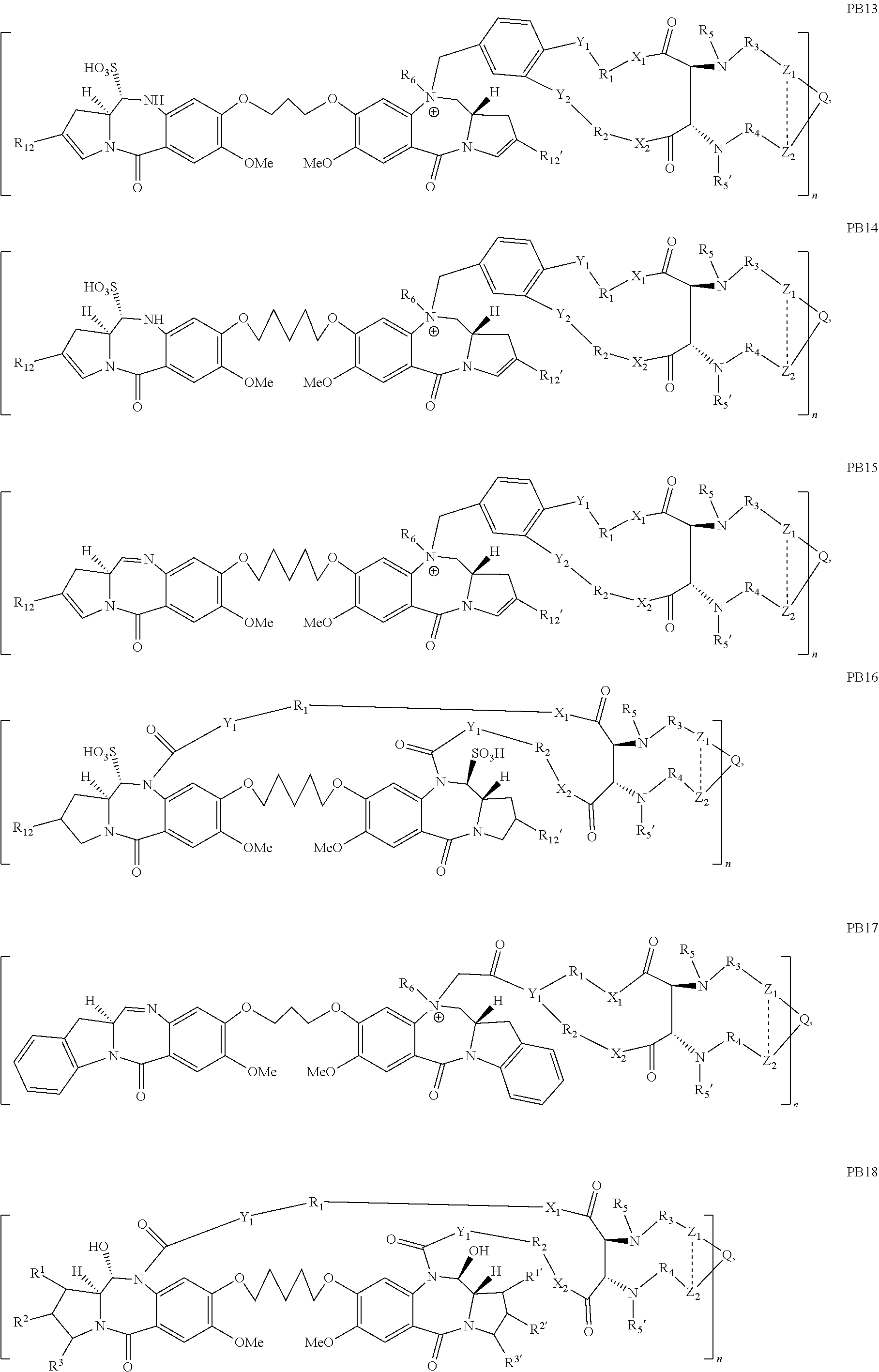 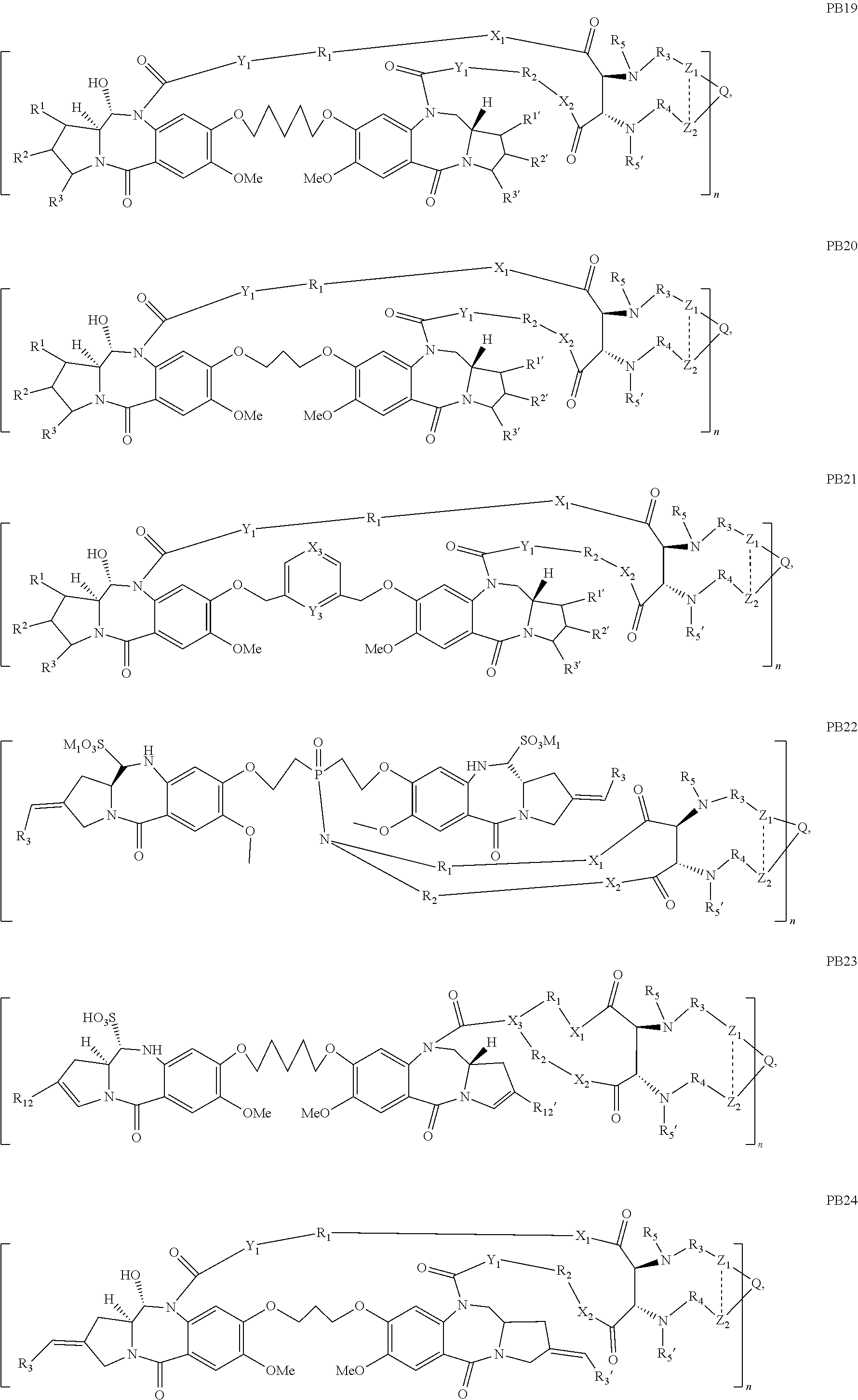 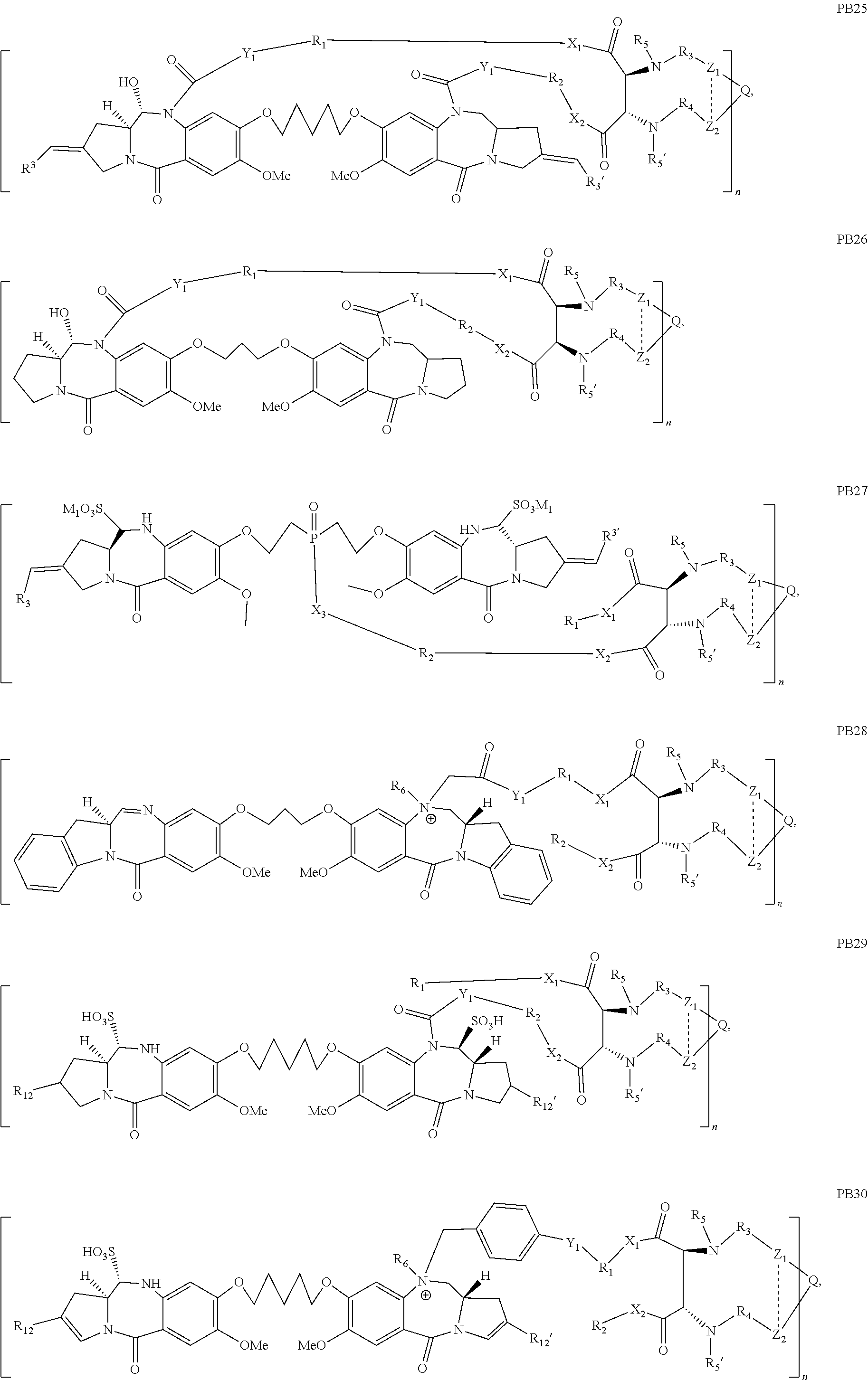 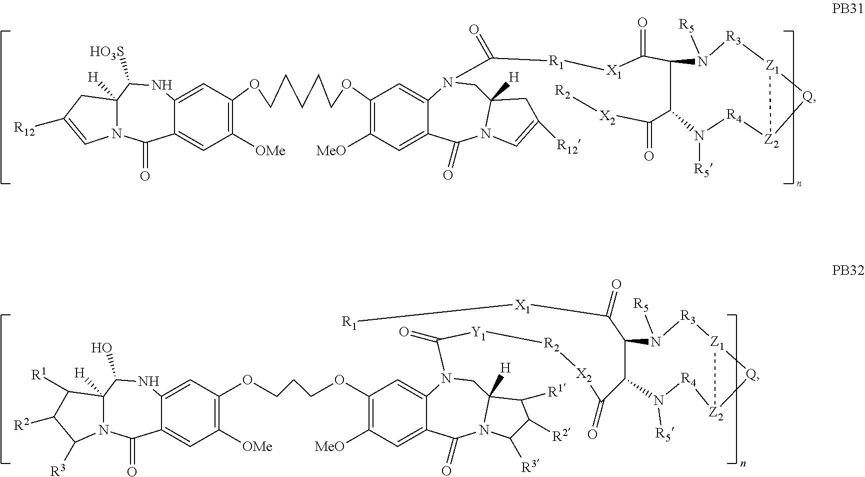
wherein “-----”, Q, X₁, X₂, Y₁, Y₂, R₁, R₂, R₃, R₄, R₅, R_{5′}, Z₁, Z₂, and n are defined the same as in claim 48; or X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R¹, R², R³, R^{1′}, R², and R^{3′} are independently H; F; Cl; ═O; ═S; OH; SH; C₁-C₈ lineal or branched alkyl, aryl, alkenyl, heteroaryl, heteroalkyl, alkylcycloalkyl, ester (COOR₅ or —OC(O)R₅), ether (OR₅), amide (CONR₅), carbamate (OCONR₅), amines (NHR₅, NR₅R_{5′}), heterocycloalkyl, or acyloxylamines (—C(O)NHOH, —ONHC(O)R₅); or peptides containing 1-20 natural or unnatural aminoacids, or polyethyleneoxy unit of formula (OCH₂CH₂)ₚ or (OCH₂CH(CH₃))ₚ, wherein p is an integer from 1 to about 5000; two Rs: R¹R₂, R²R₃, R¹R₃, R^{1′}R^{2′}, R^{2′}R^{3′}, or R^{1′}R^{3′} can independently form 3-8 member cyclic ring of alkyl, aryl, heteroaryl, heteroalkyl, or alkylcycloalkyl group; X₃ and Y₃ are independently N, NH, CH₂ or CR₅, wherein R₅, R₆, R₁₂ and R₁₂′ are independently H, OH, NH₂, NH(CH₃), NHNH₂, COOH, SH, OZ₃, SZ₃, F, Cl, or C₁-C₈lineal or branched alkyl, aryl, heteroaryl, heteroalkyl, alkylcycloalkyl, acyloxylamines; Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, or NR₁R₂R₃.

73. The conjugate compound of claim 48, wherein the Drug, or Drug₂ is an amanitin analogue, the conjugate compound is selected from structures of Am01, Am02, Am03, Am04, Am05, Am06, Am07, Am08 and Am09 below: 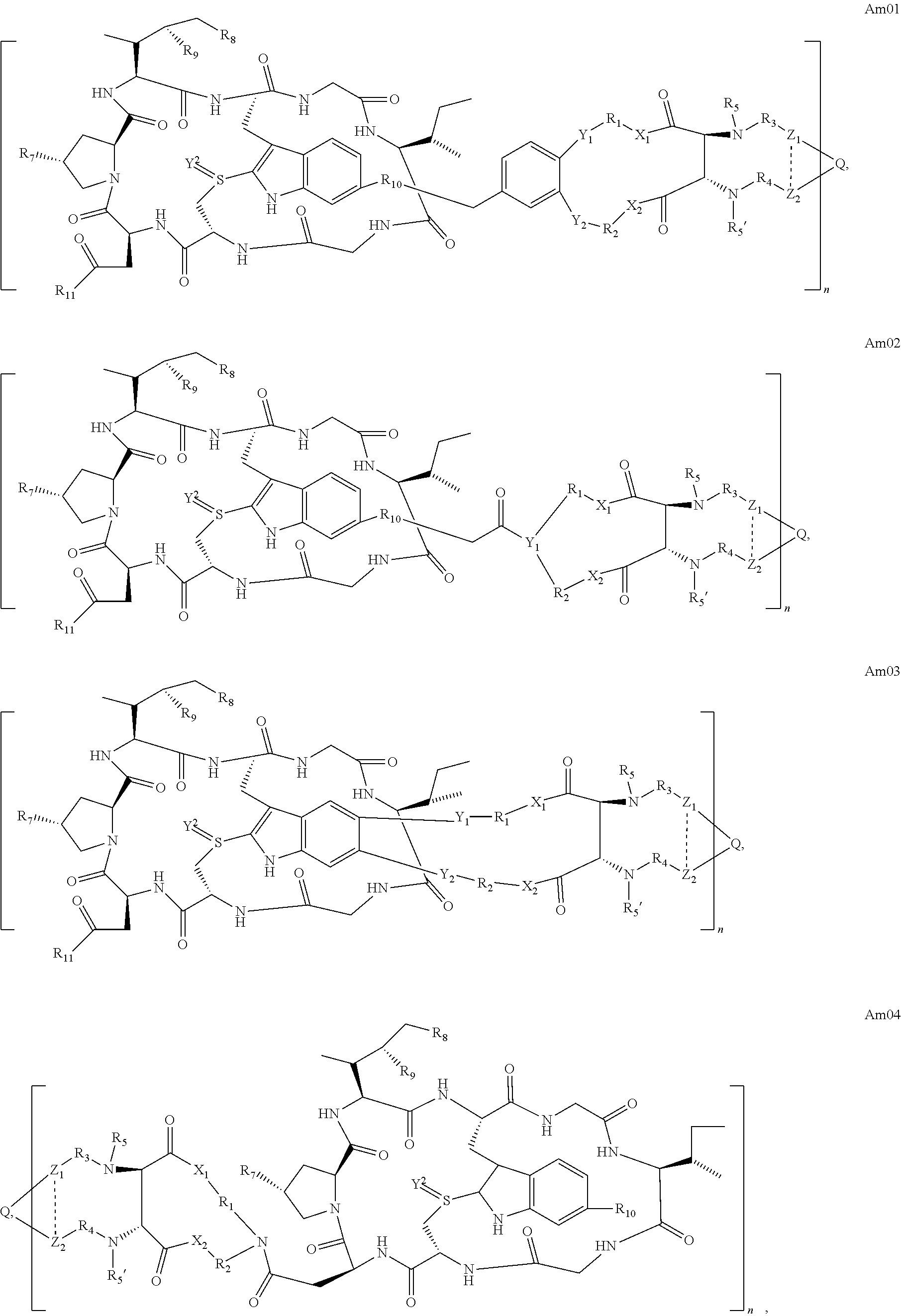 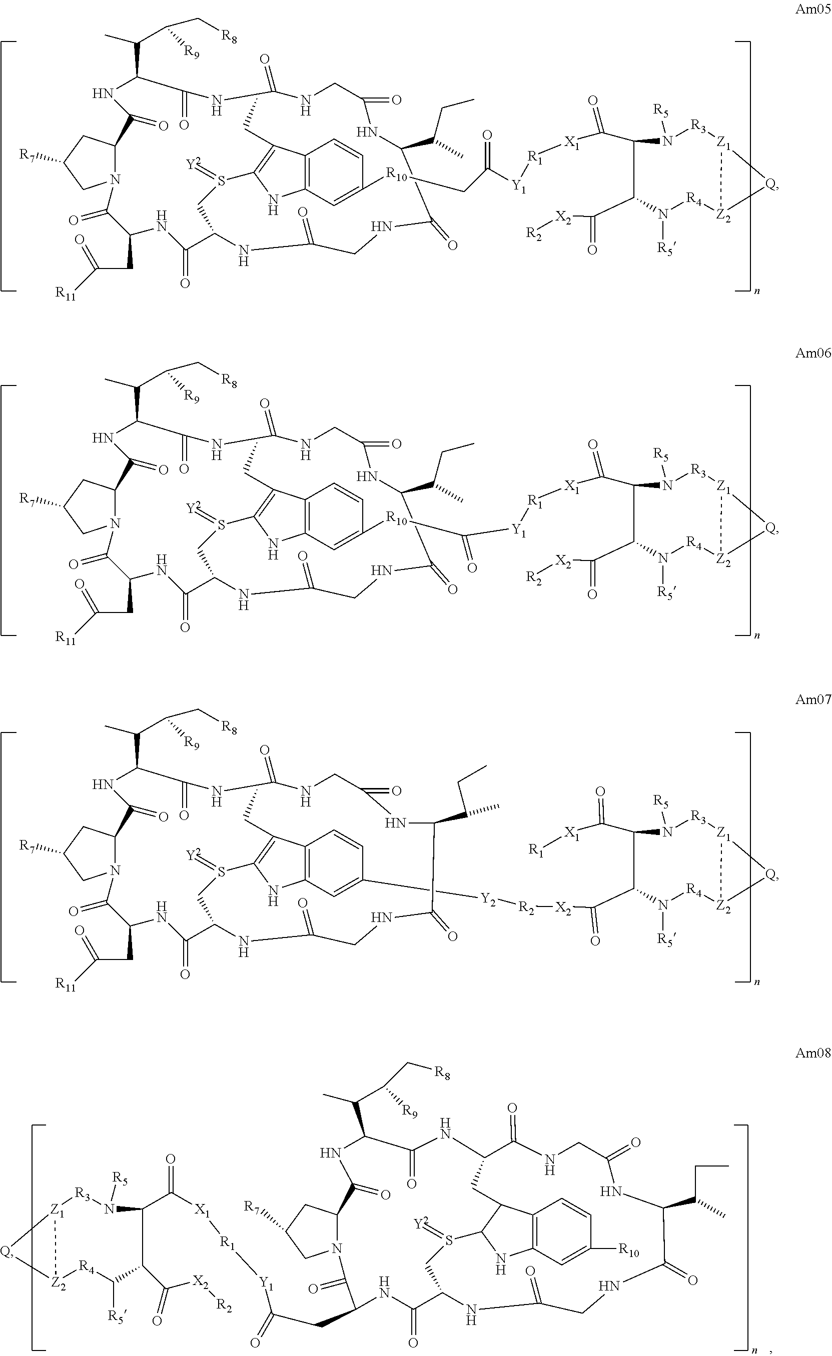 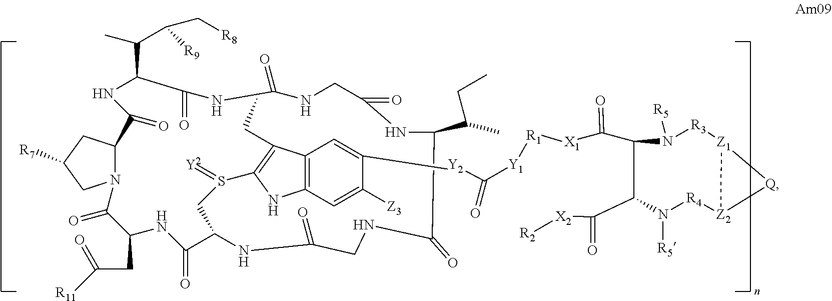
wherein “-----”, X₁, X₂, Q, Y₁, Y₂, R₁, R₂, R₃, R₄, R₅, R_{5′}, Z₁, Z₂, and n are defined the same as in claim 48; or X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NH—NHC(O), C(O)NR₁ or absent; R₇, R₃, and R₉ are independently H, OH, OR₁, NH₂, NHR₁, C₁-C₆ alkyl, or absent; Y² is O, O₂, NR₁, NH, or absent; R₁₀ is CH₂, O, NH, NR₁, NHC(O), NHC(O)—NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂CH₂NHPO₃H₂, wherein Aa is 1-8 aminoacids; n and m₁ are independently 1-30; p is 1-5000; Z₃ is H, OH, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M1, R₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, or NR₁R₂R₃.

74. The conjugate compound of claim 48, wherein the Drug₁ or Drug₂ is a camptothecin and its derivative, the conjugate compound is selected from structures of CP01, CP02, CP03, CP04, CP05, and CP06 below: 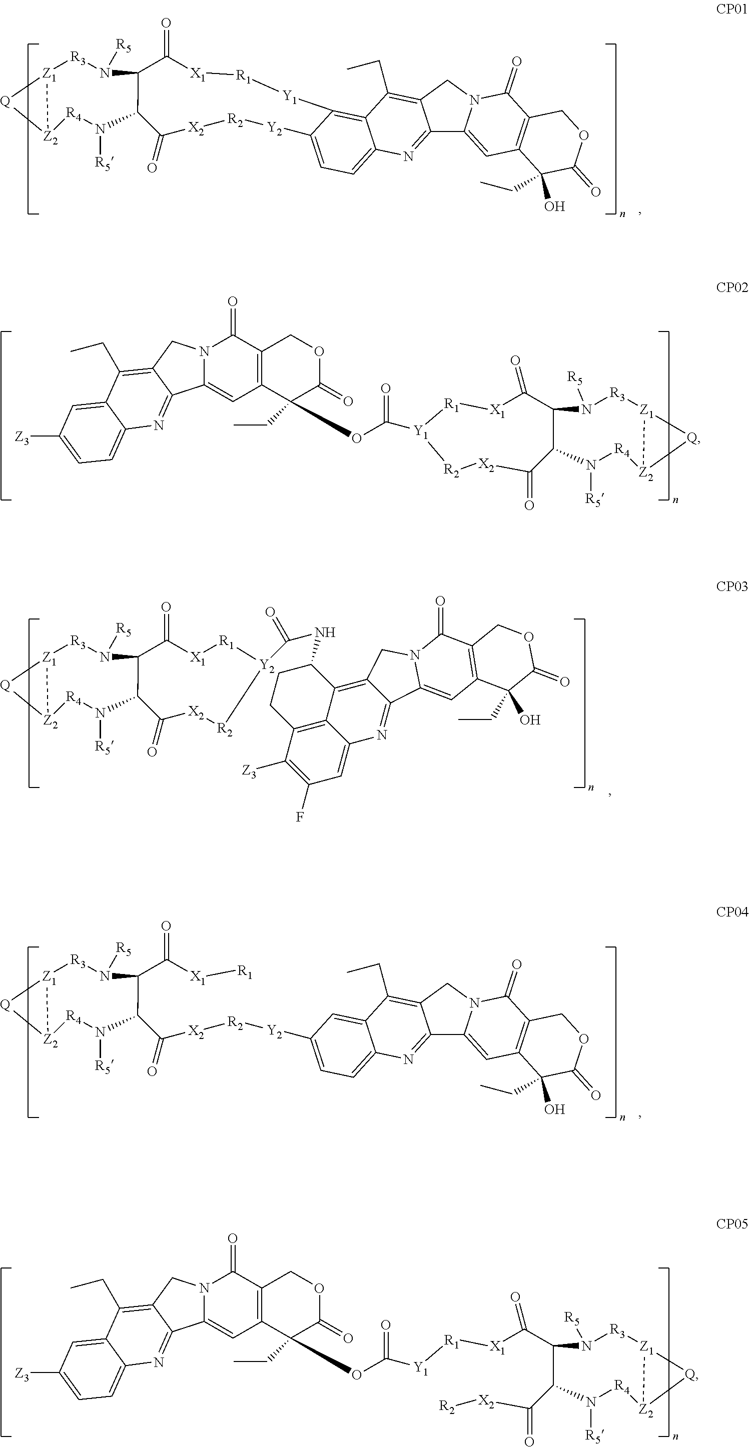 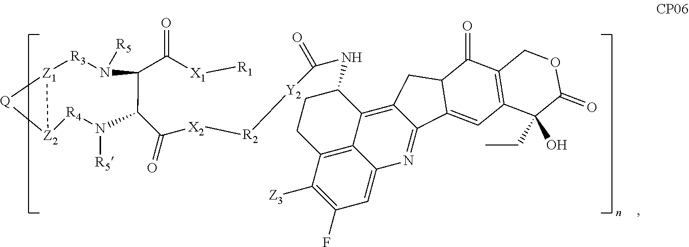
wherein “---”, Q, X₁, X₂, Y₁, Y₂, R₁, R₂, R₃, R₄, R₅, R_{5′}, Z₁, Z₂, and n are defined the same as in claim 48; or X₁, X₂, Y₁ and Y₂ are independently O, N, NH, NHNH, NR, S, C(O)O, C(O)NH, OC(O)NH, OC(O)O, NHC(O)NH, NHC(O)S, OC(O)N(R₁), N(R₁)C(O)N(R₁), CH, CH₂, C(O)NHNHC(O), C(O)NR₁ or absent; Z₃ is H, OH, COOR₁, NH₂, NHR₁, OR₁, CH₃, CONHR₁,NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, or NR₁R₂R₃.

75. The conjugate compound of claim 48, wherein the Drug₁ or Drug₂ is an eribulin and its derivative, the conjugate compound is selected from structures of Eb01, and Eb02 below: 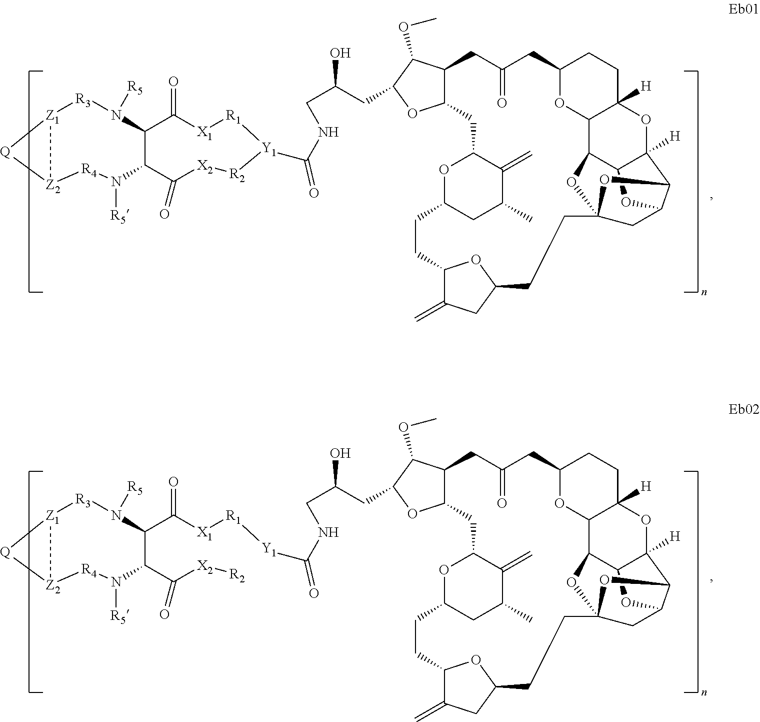
wherein “-----”, Q, X₁, X₂, Y₁, R₁, R₂, R₃, R₄, R₅, R_{5′}, Z₁, Z₂, and n are defined the same as in claim 48; or X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, CH₂, C(O)NHNHC(O), C(O)NR, or absent.

76. The conjugate compound of claim 48, wherein the Drug₁ or Drug₂ is an inhibitor of nicotinamide phosphoribosyltransferases, the conjugate compound is selected from structures of NP01, NP02, NP03, NP04, NP05, NP06, NP07, NP08, and NP09 below: 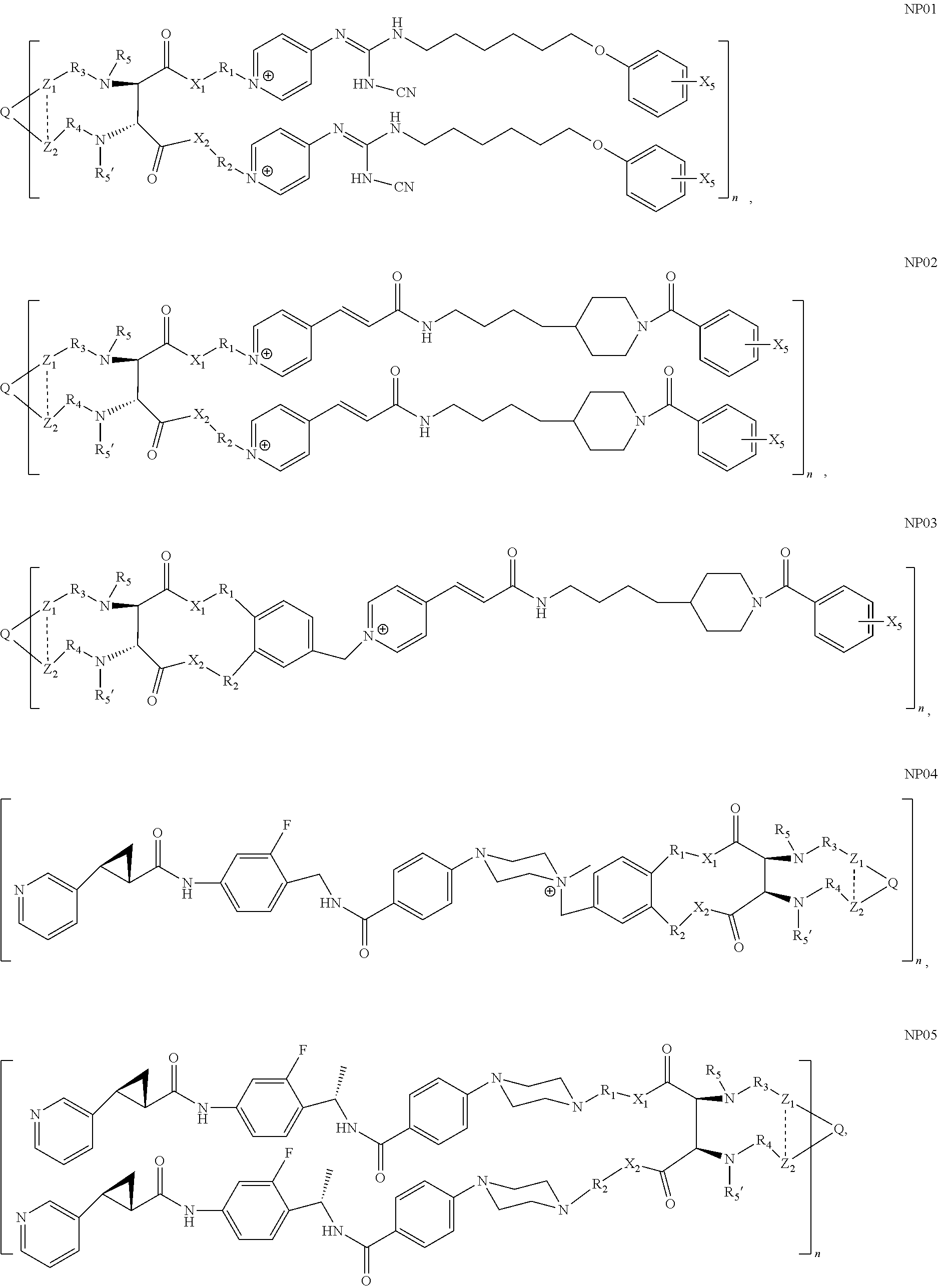 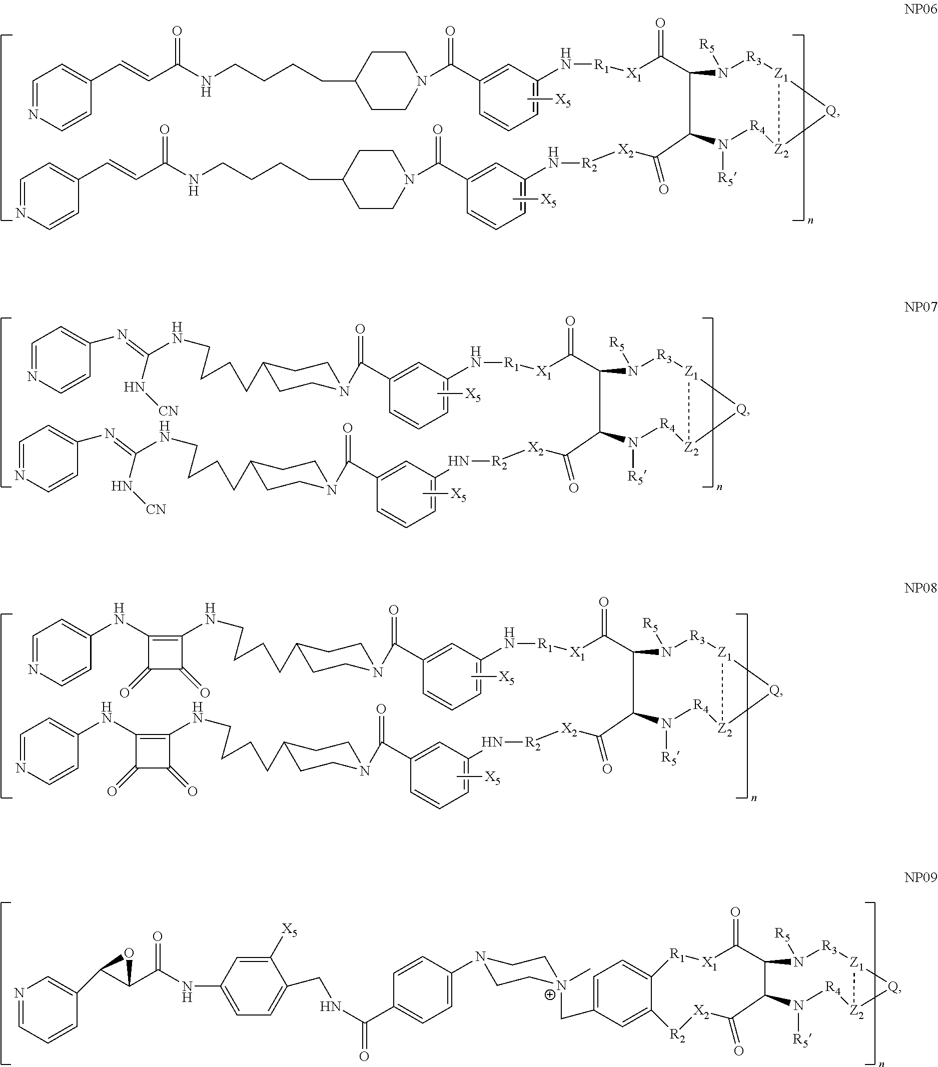
wherein “-----”, Q, X₁, X₂, Y₁, R₁, R₂, R₃, R₄, R₅, R_{5′}, Z₁, Z₂, and n are defined the same as in claim 48; X₅ is F, Cl, Br, I, OH, OR₁, R₁, OPO₃H₂, OSO₃H, NHR₁, OCOR₁, NHCOR₁; or X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, CH₂, C(O)NHNHC(O), C(O)NR₁ or absent.

77. The conjugate compound of claim 48, wherein the Drug, or Drug₂ is a polyalkylene glycol analog, the conjugate compound is selected from structures of Pg01, Pg02, and Pg03: 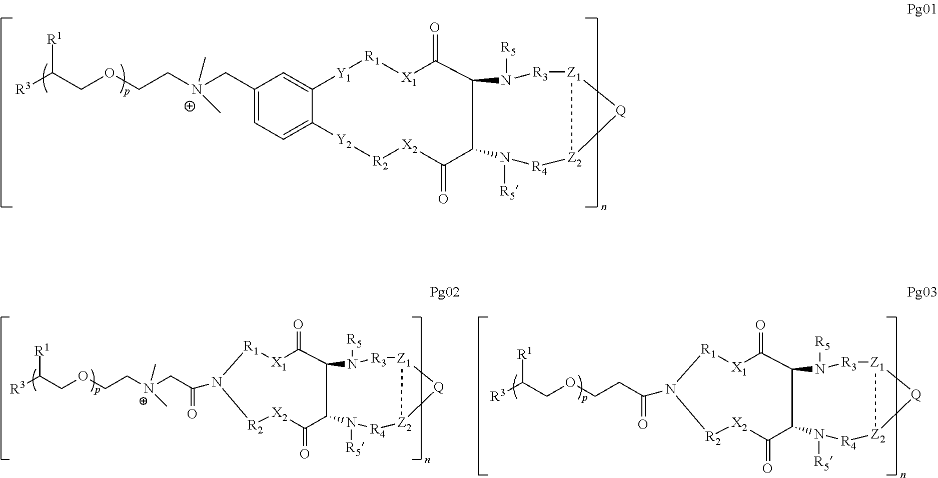
wherein “-----”, Q, X₁, X₂, Y₁, Y₂, R₁, R₂, R₃, R₄, R₅, R_{5′}, Z₁, Z₂, and n are defined the same as in claim 48; or Y, and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NH—NHC(O) and C(O)NR₁; p is 1-5000; R¹ and R³ are defined the same as R, above.

78. The conjugate compound of claim 48, wherein Drug, or Drug₂ is a cell-binding ligand or cell receptor agonist and its analog, the conjugate compound is selected from structures of: LB01 (Folate conjugate), LB02 (PMSA ligand conjugate), LB03 (PMSA ligand conjugate), LB04 (PMSA ligand conjugate), LB05 (Somatostatin conjugate), LB06 (Somatostatin conjugate), LB07 (Octreotide, a Somatostatin analog conjugate), LB08 (Lanreotide, a Somatostatin analog conjugate), LB09 (Vapreotide (Sanvar), a Somatostatin analog conjugate), LB10 (CAIX ligand conjugate), LB11 (CAIX ligand conjugate), LB12 (Gastrin releasing peptide receptor (GRPr), MBA conjugate), LB13 (luteinizing hormone-releasing hormone (LH-RH) ligand and GnRH conjugate), LB14 (luteinizing hormone-releasing hormone (LH-RH) and GnRH ligand conjugate), LB15 (GnRH antagonist, Abarelix conjugate), LB16 (cobalamin, vitamin B12 analog conjugate), LB17 (cobalamin, vitamin B12 analog conjugate), LB18 (for αᵥβ₃ integrin receptor, cyclic RGD pentapeptide conjugate), LB19 (hetero-bivalent peptide ligand conjugate for VEGF receptor), LB20 (Neuromedin B conjugate), LB21 (bombesin conjugate for a G-protein coupled receptor), LB22 (TLR₂ conjugate for a Toll-like receptor,), LB23 (for an androgen receptor), LB24 (Cilengitide/cyclo(-RGDfV-) conjugate for an α_{V} intergrin receptor, LB23 (Fludrocortisone conjugate), LB25 (Rifabutin analog conjugate), LB26 (Rifabutin analog conjugate), LB27 (Rifabutin analog conjugate), LB28 (Fludrocortisone conjugate), LB29 (Dexamethasone conjugate), LB30 (fluticasone propionate conjugate), LB31 (Beclometasone dipropionate conjugate), LB32 (Triamcinolone acetonide conjugate), LB33 (Prednisone conjugate), LB34 (Prednisolone conjugate), LB35 (Methylprednisolone conjugate), LB36 (Betamethasone conjugate), LB37 (Irinotecan analog conjugate), LB38 (Crizotinib analog conjugate), LB39 (Bortezomib analog conjugate), LB40 (Carfilzomib analog conjugate), LB41 (Carfilzomib analog conjugate), LB42 (Leuprolide analog conjugate), LB43 (Triptorelin analog conjugate), LB44 (Clindamycin conjugate), LB45 (Liraglutide analog conjugate), LB46 (Semaglutide analog conjugate), LB47 (Retapamulin analog conjugate), LB48 (Indibulin analog conjugate), LB49 (Vinblastine analog conjugate), LB50 (Lixisenatide analog conjugate), LB51 (Osimertinib analog conjugate), LB52 (a neucleoside analog conjugate), LB53 (Erlotinib analog conjugate) and LB54 (Lapatinib analog conjugate) as shown in the following structures: 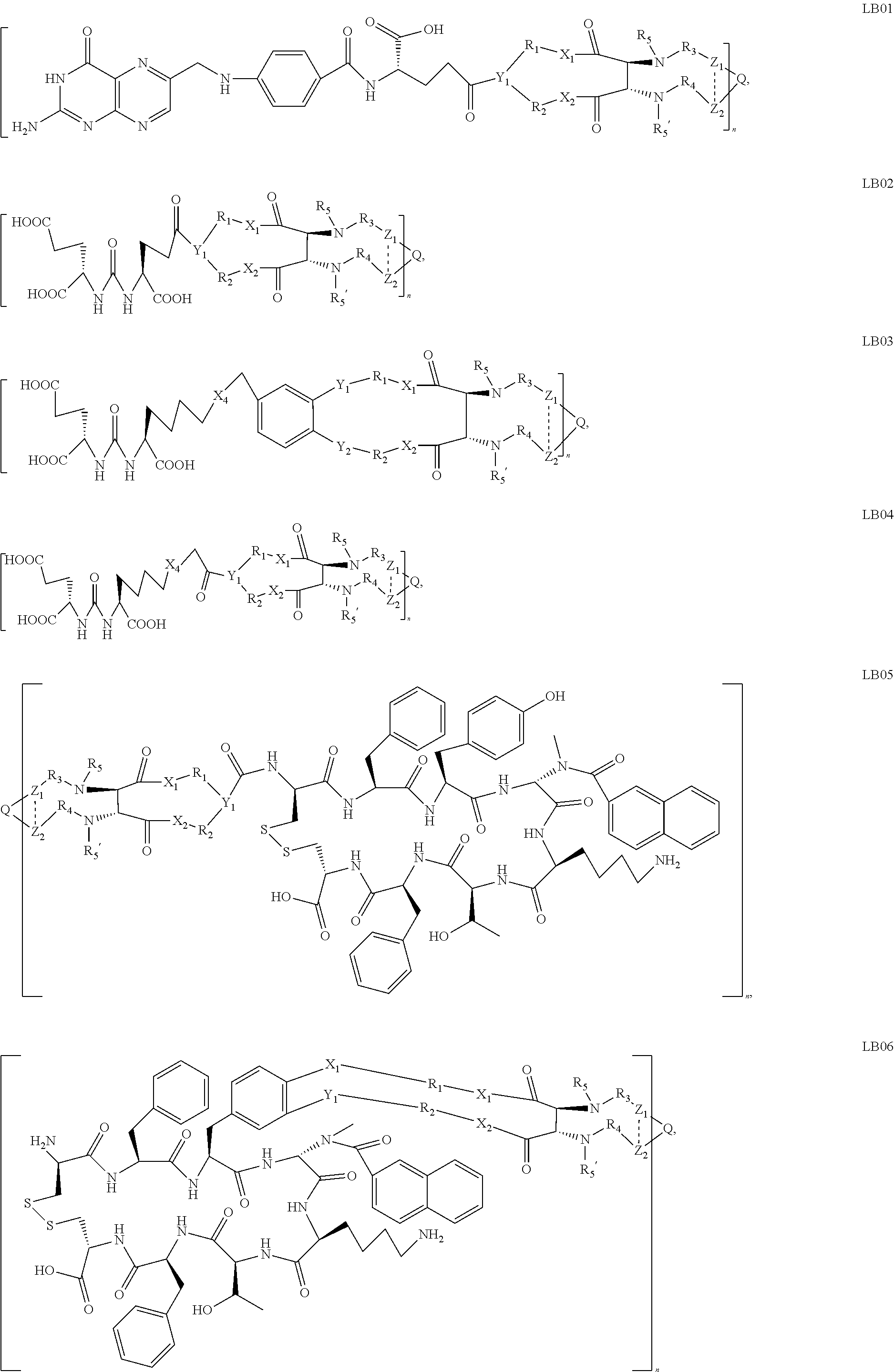 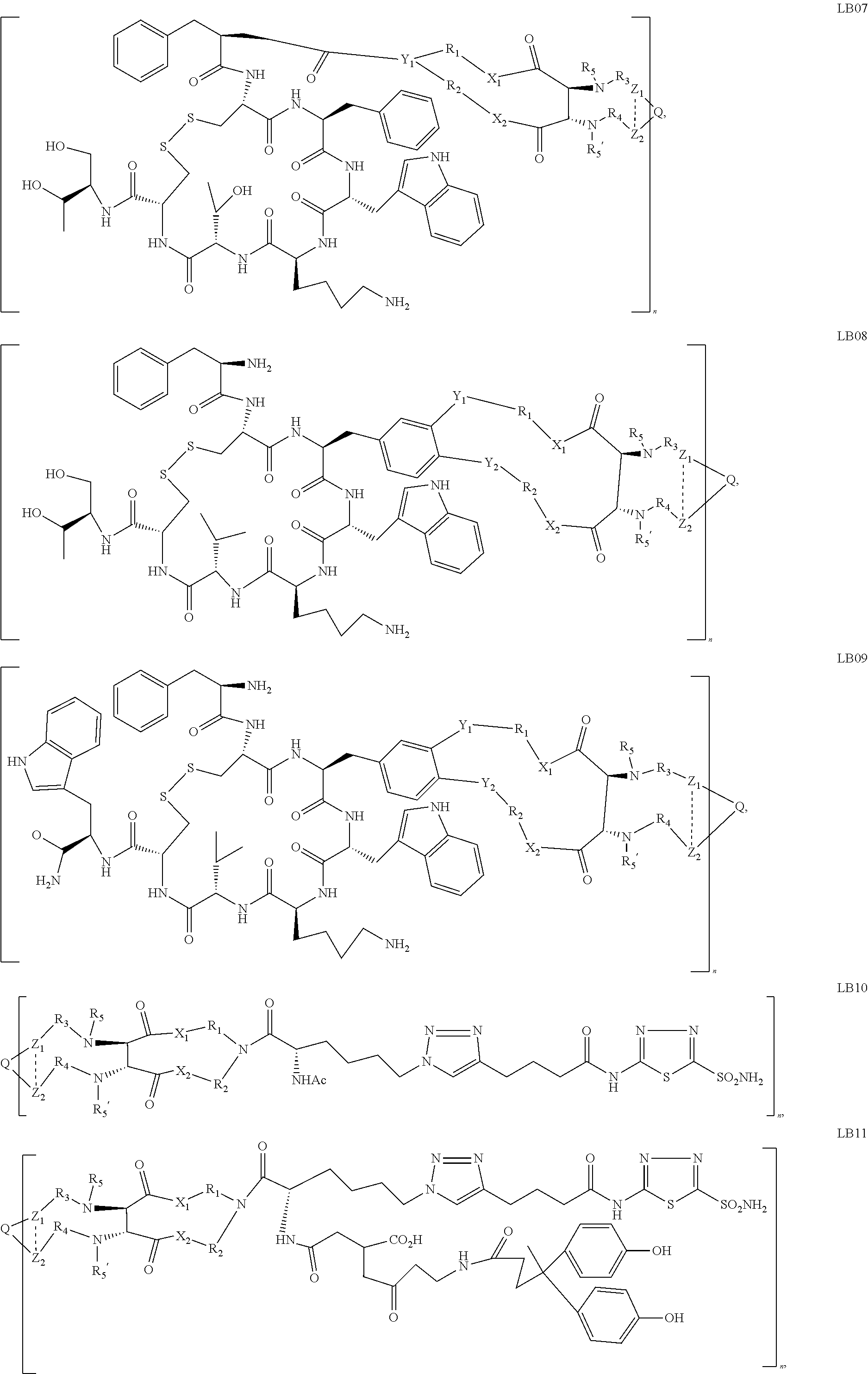 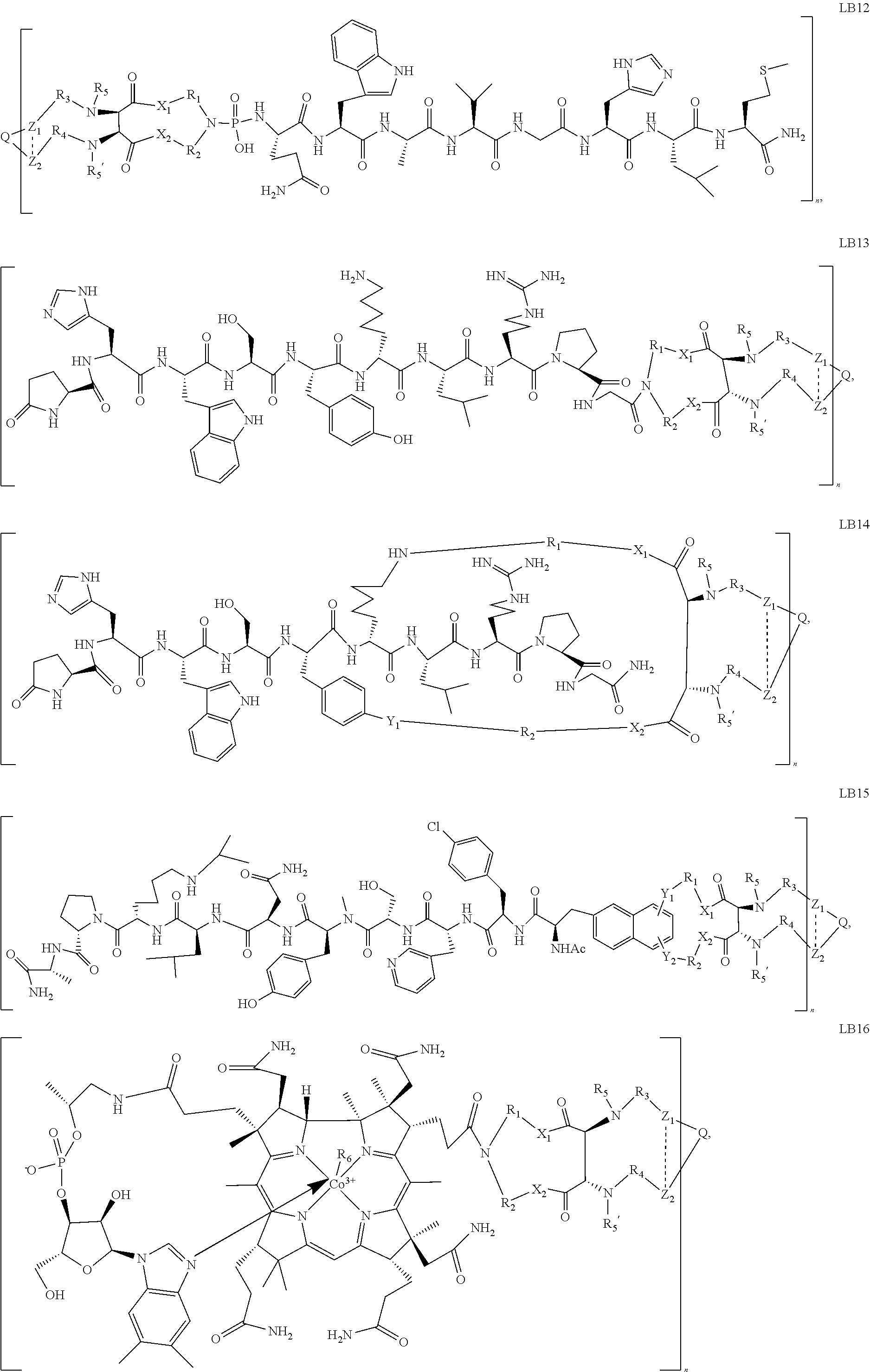 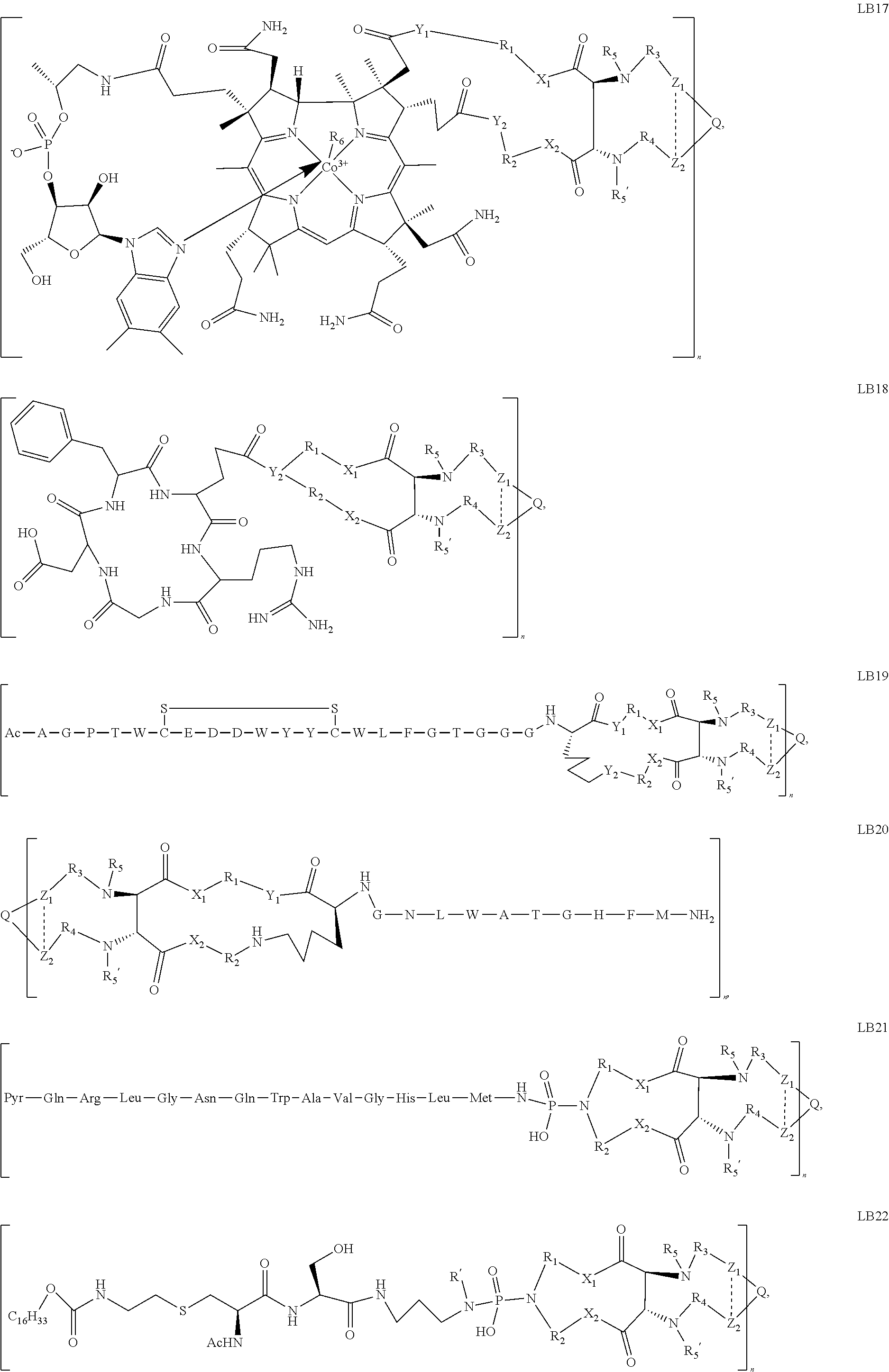 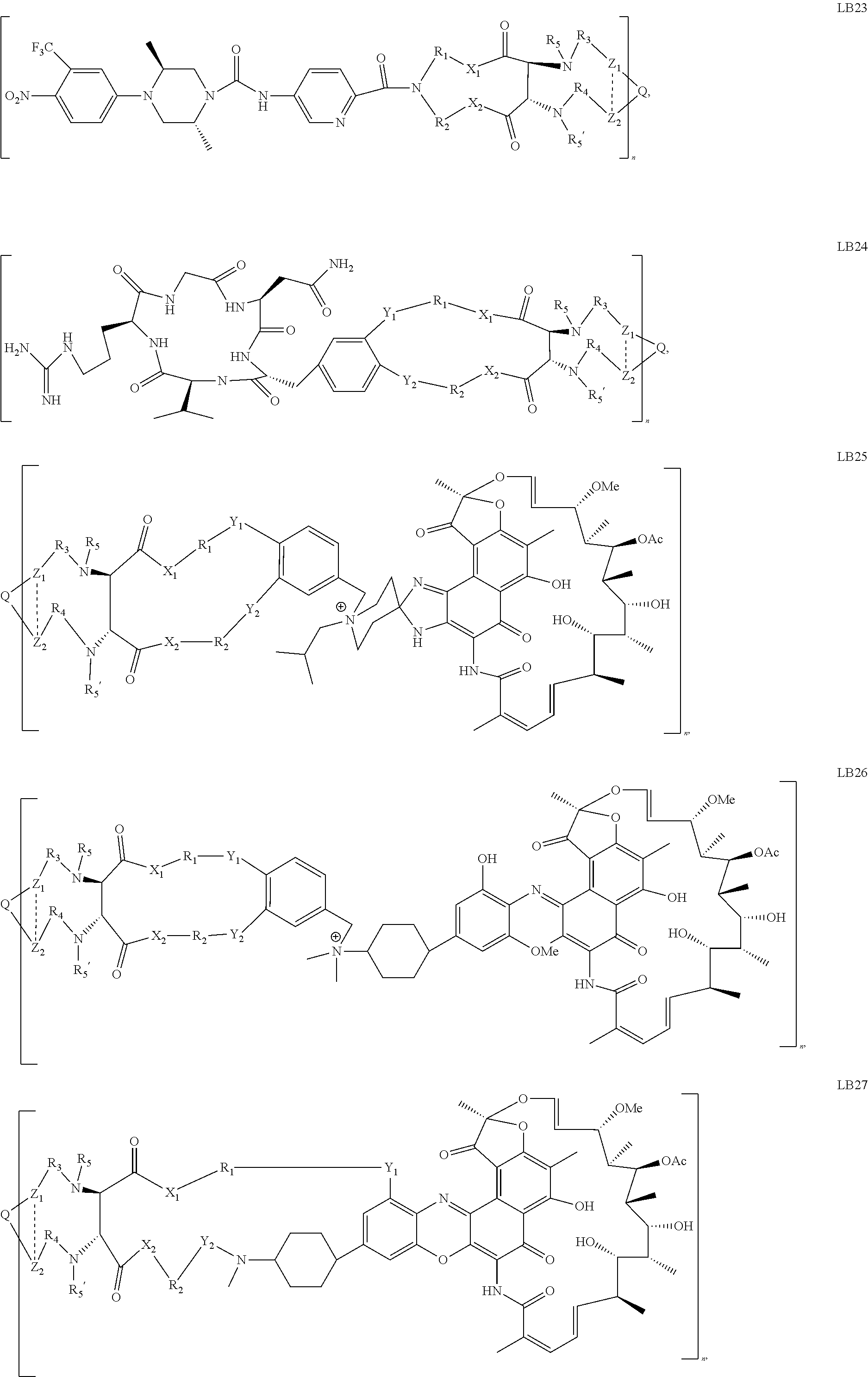 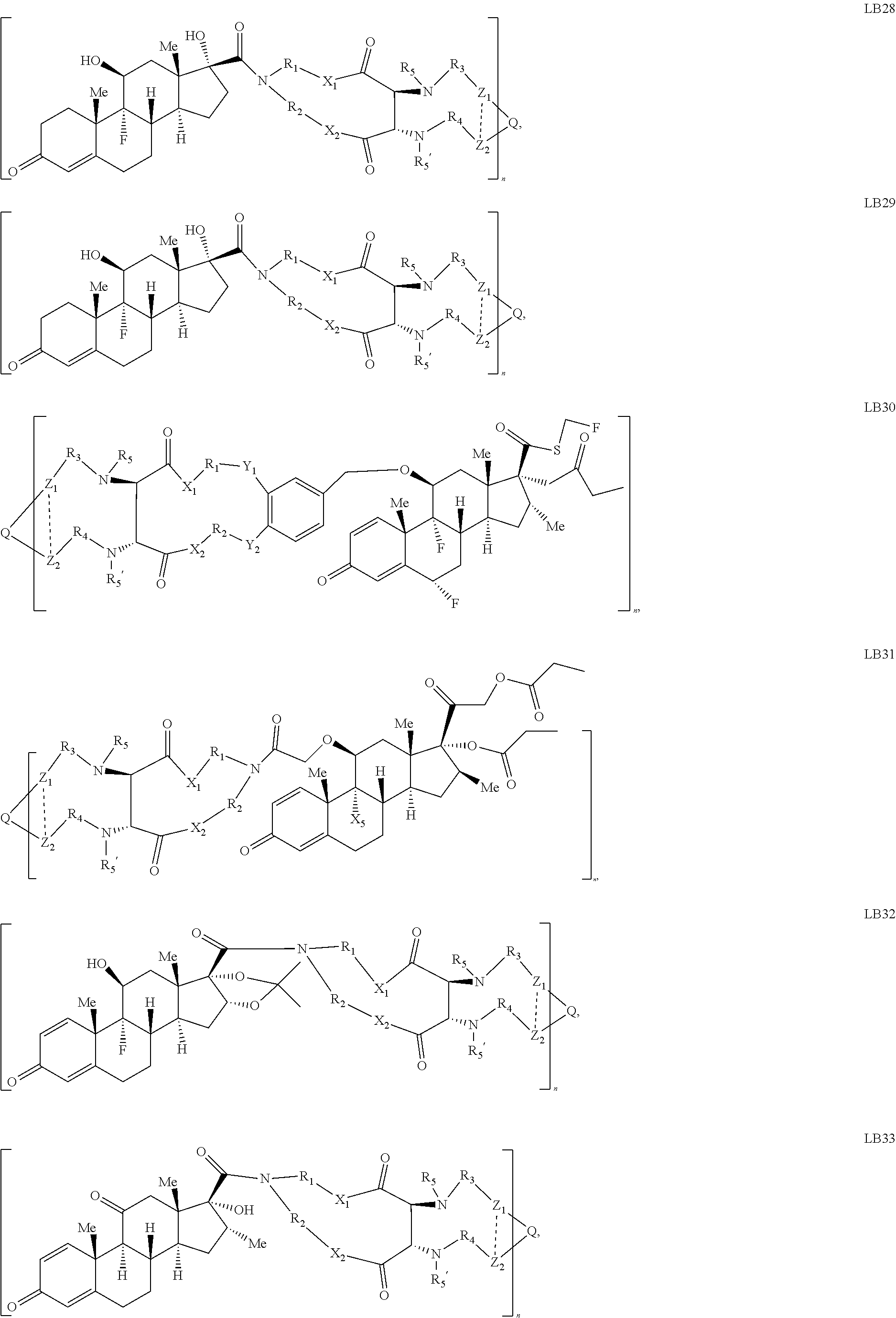 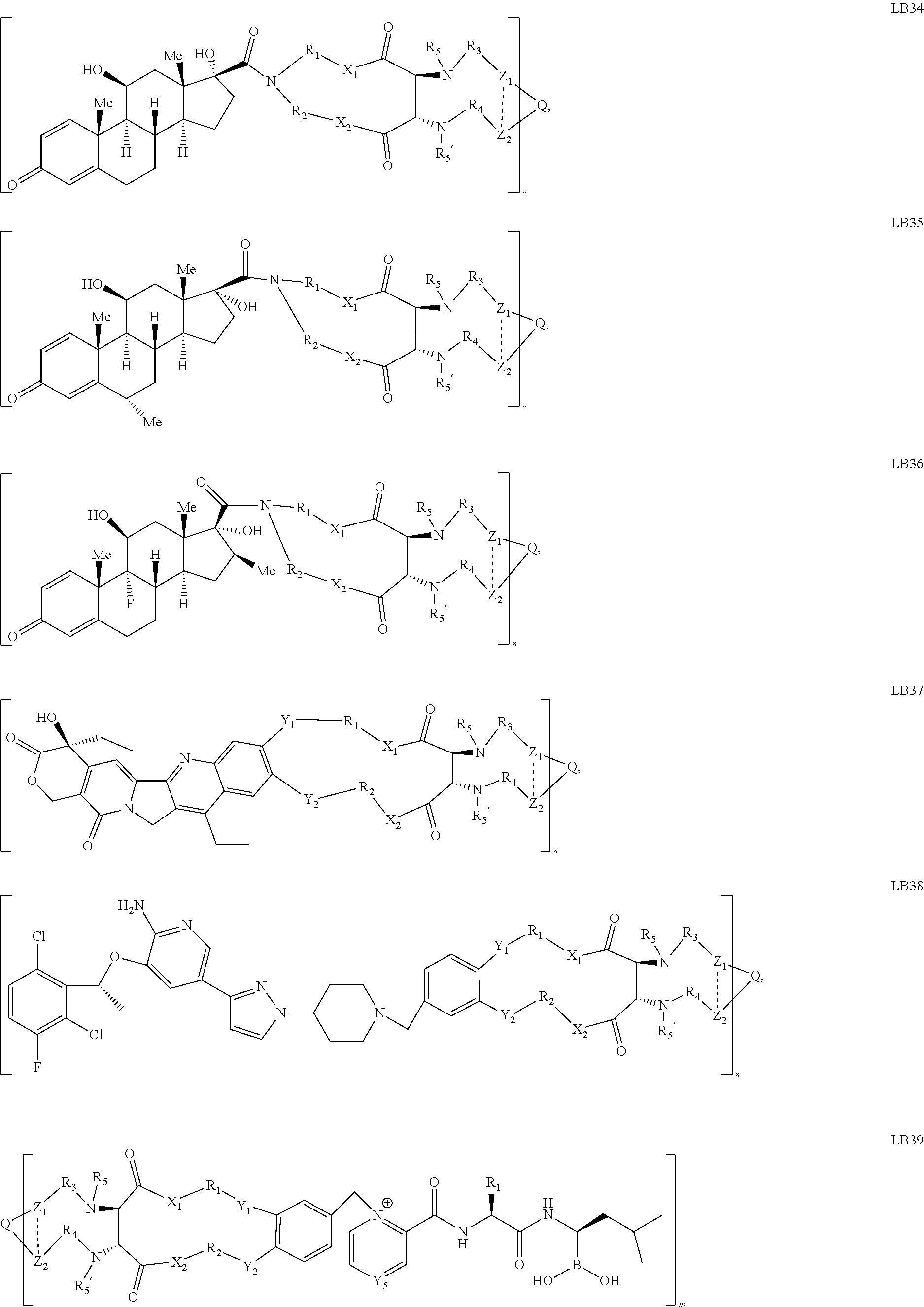
wherein Y₅, is N, OH, C(Cl), C(CH₃), or C(COOR₁); R₁ is H, C₁-C₆ alkyl, C₃-C₈ Ar, 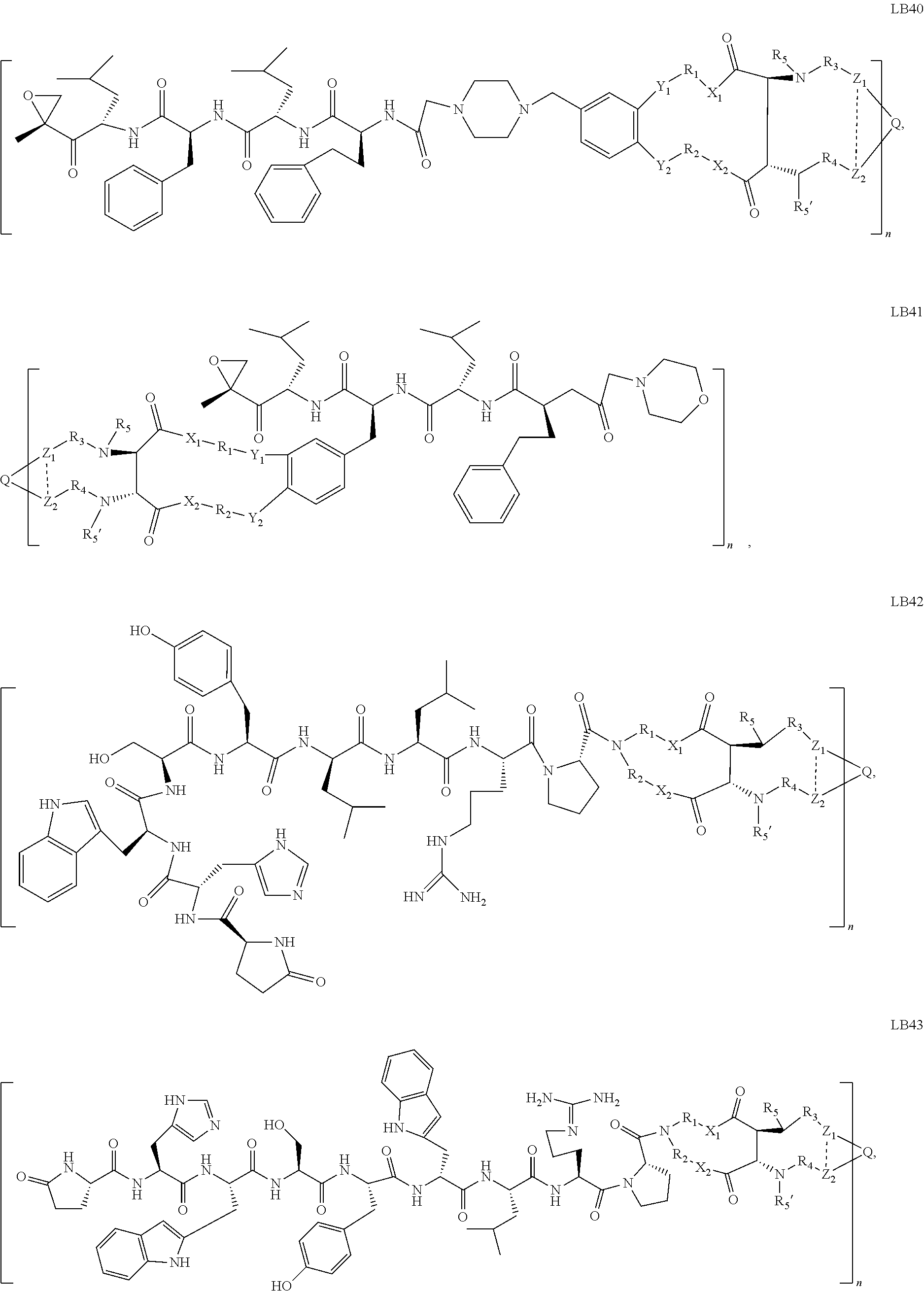 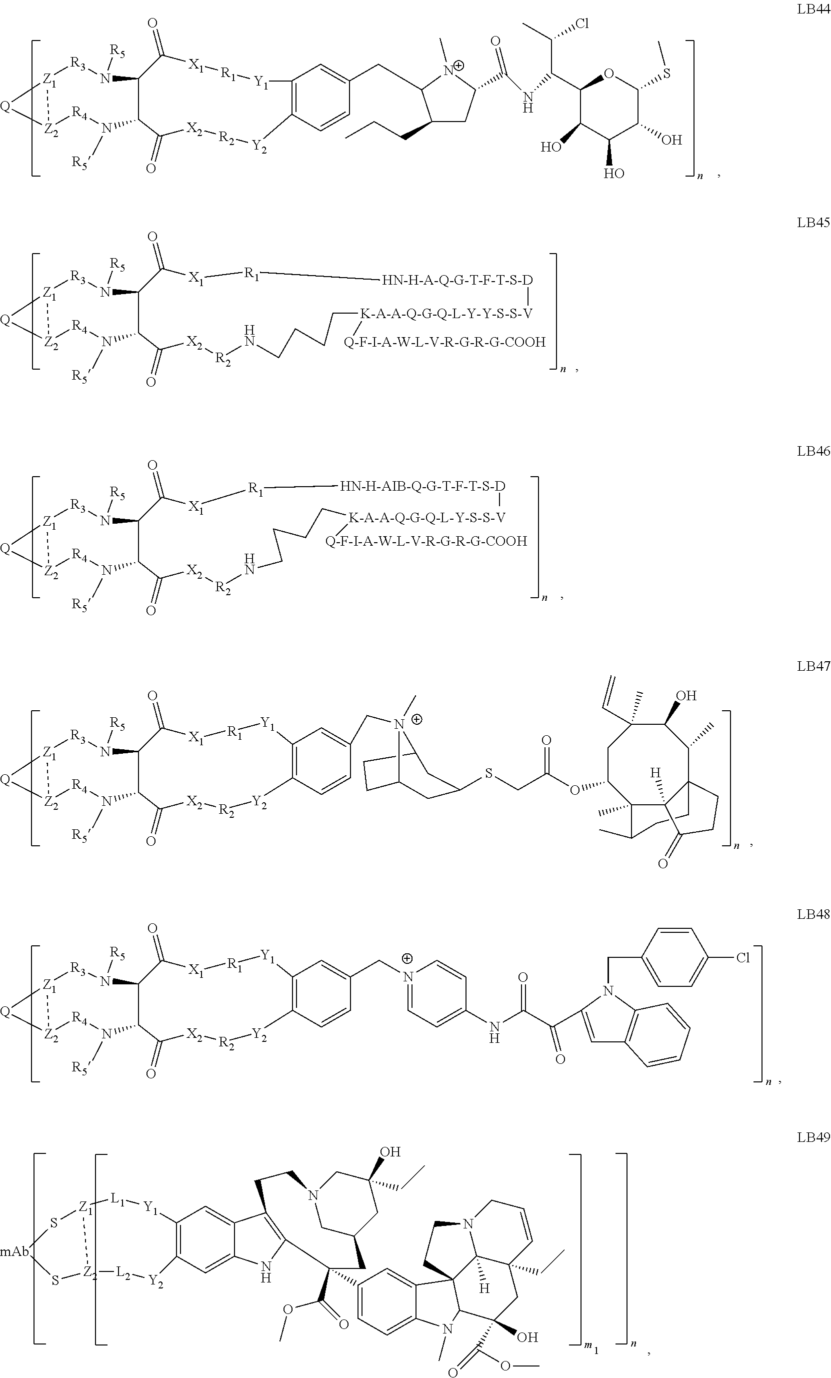 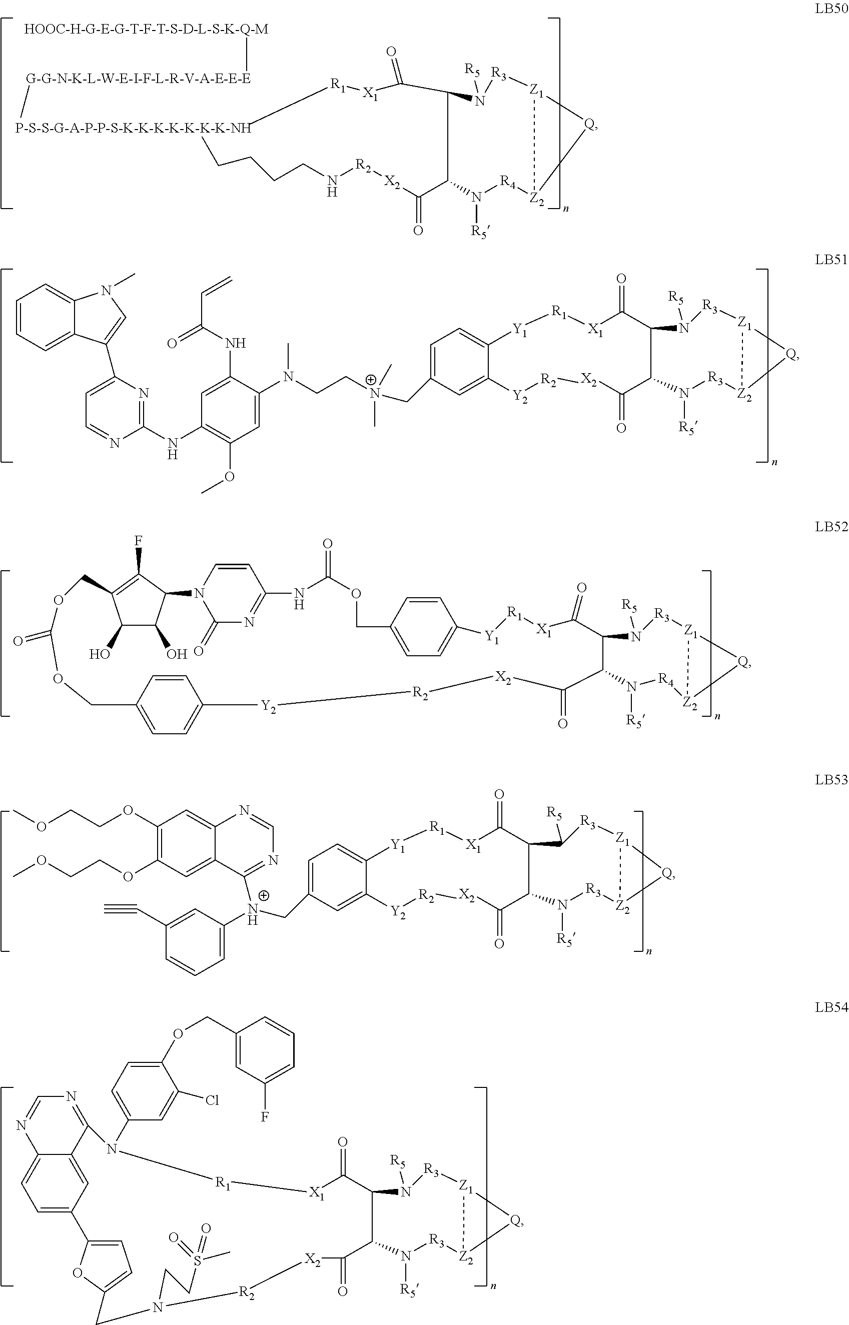
wherein “-----”, X₁, X₂, Q, Y₁, Y₂, R₁, R₂, R₃, R₄, R₅, R_{5′}, Z₁, Z₂, and n are defined the same as in claim 48; or X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; X₃ is CH₂, O, NH, NHC(O), NHC(O)NH, C(O), OC(O), OC(O)(NR₃), R₁, NHR₁, NR₁, C(O)R, or absent; X₄ is H, CH₂, OH, O, C(O), C(O)NH, C(O)N(R₁), R₁, NHR₁, NR₁, C(O)R₁ or C(O)O; X₅ is H, CH₃, F, or C₁; M₁ and M₂ are independently H, Na, K, Ca, Mg, NH₄, or NR₁R₂R₃; R₆ is 5′-deoxyadenosyl, Me, OH, or CN.

79. The conjugate compound of claim 48, wherein the cytotoxic molecule is a DNA, RNA, mRNA, small interfering RNA (siRNA), microRNA (miRNA), or PIWI interacting RNAs (piRNA), the conjugate compound is selected from structure of SI-1 below 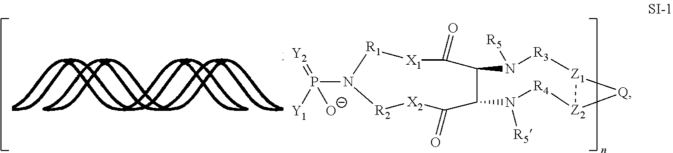
wherein “-----”, Q, X₁, X₂, Y₁, Y₂, R₁, R₂, R₃, R₄, R₅, R_{5′}, Z₁, Z₂, and n are defined the same as in claim 48; or X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, CH₂, C(O)NHNHC(O) and C(O)NR₁; [symbol] is single or double strands of DNA, RNA, mRNA, siRNA, miRNA, or piRNA.

80. The conjugate compound according to claim 48, wherein the cell-binding agent is selected from an IgG antibody, monoclonal antibody, or an IgG antibody-like protein, having the following structure of ST1, ST2, ST3, ST4, ST5, or ST6 below, wherein the conjugate is conjugated specifically to a pair of thiols generated through reduction of the disulfide bonds of the cell-binding molecule/agent between the light chain and heavy chain, the upper disulfide bonds between the two heavy chains and the lower disulfide bonds between the two heavy chains: 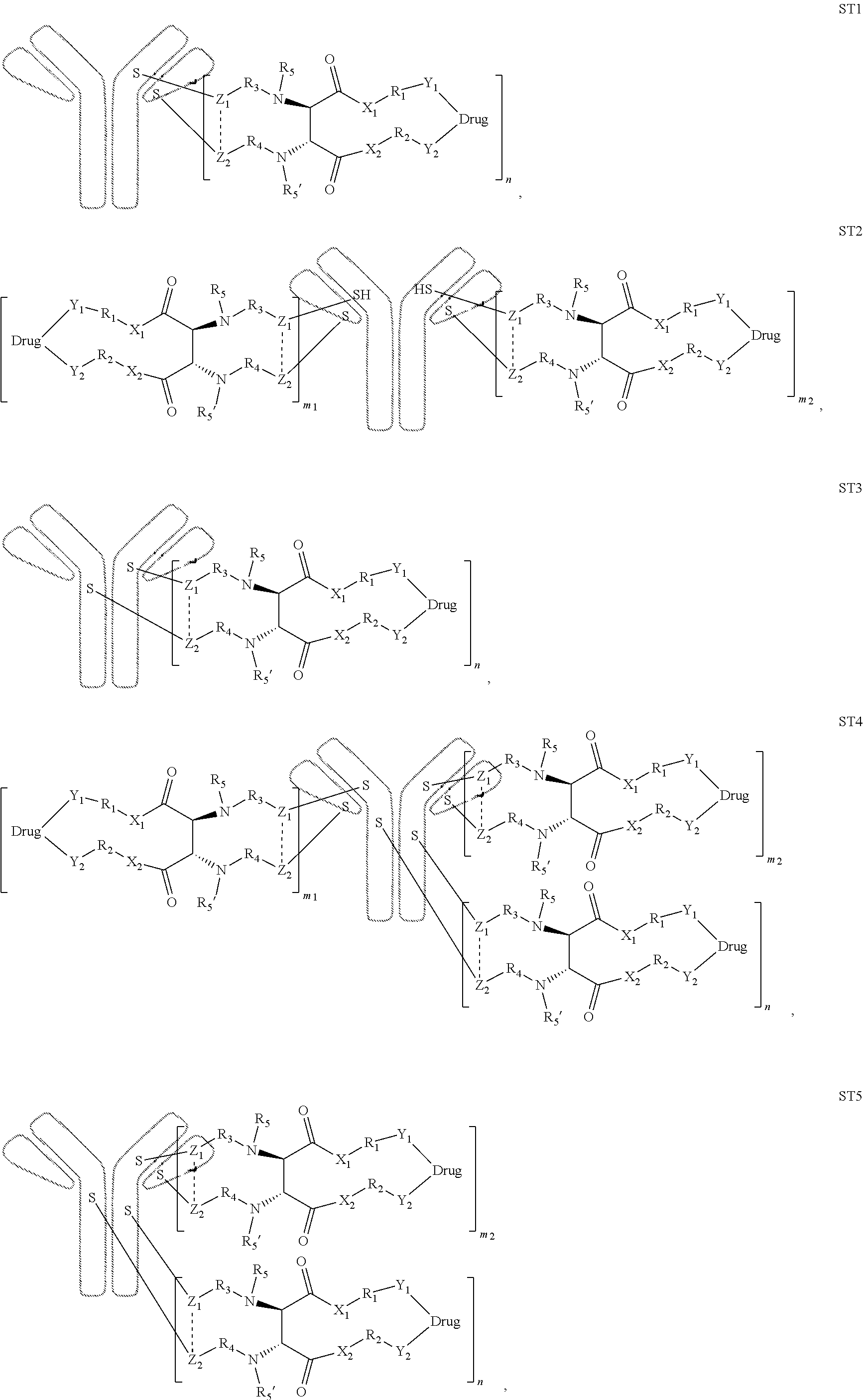 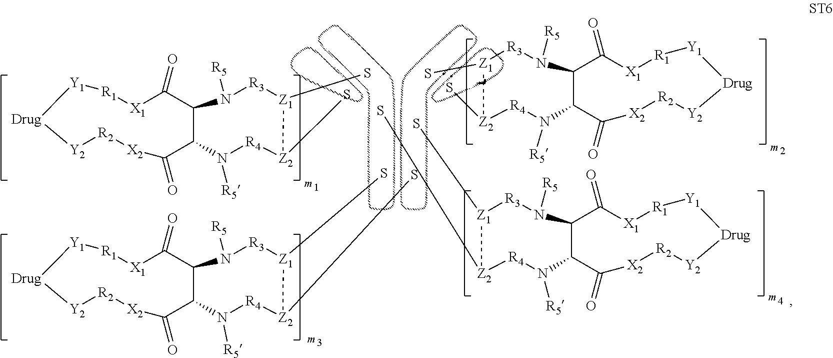
wherein “-----”, Y₁, Y₂, R₁, R₂, R₃, R₄, R₅, R_{5′}, Z₁, Z₂, and n are defined the same as in claim 48; or X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), OH, CH₂, C(O)NHNHC(O) and C(O)NR₁; ml, m₂, m₃, and m₄ are independently 1-30.

81. The conjugate compound according to claim 80, wherein the Drug or cytotoxic agent and m₁ at different conjugation site of the cell-binding molecule can be different when the cytotoxic molecules containing the same or different bis-linkers are conjugated to a cell-binding molecule sequentially or stepwise.

82. The conjugate compound according to claim 80, wherein the Drug is tubulysins, maytansinoids, taxanoids (taxanes), CC-1065 analogs, daunorubicin and doxorubicin compounds, indolecarboxamide, benzodiazepine dimers, pyrrolobenzodiazepine (PBD) dimers, tomaymycin dimers, anthramycin dimers, indolinobenzodiazepines dimers, imidazobenzothiadiazepines dimers, oxazolidinobenzodiazepines dimers, calicheamicins and the enediyne antibiotics, actinomycin, amanitins, amatoxins, azaserines, bleomycins, epirubicin, eribulin, tamoxifen, idarubicin, dolastatins, auristatins (comprising monomethyl auristatin E, MMAE, MMAF, auristatin PYE, auristatin TP, Auristatins 2-AQ, 6-AQ, EB (AEB), EFP (AEFP) and their analogs), duocarmycins, geldanamycins, HSP90 inhibitors, inhibitor of nicotinamide phosphoribosyltransferases, centanamycin, methotrexates, thiotepa, vindesines, vincristines, hemiasterlins, nazumamides, microginins, radiosumins, streptonigtin, SN38 or other analogs or metabolites of camptothecin, alterobactins, microsclerodermins, theonellamides, esperamicins, PNU-159682, and their analogues or derivatives, pharmaceutically acceptable salts, acids, derivatives, hydrate or hydrated salt, a crystalline structure, an optical isomer, racemate, diastereomer or enantiomer of any of the above drugs thereof; or a cytotoxic compound.

83. The method according to claim 50, wherein the stereoisomeric compound has the formula of A-01, A-02, A-03, A-04, B-01, B-02, B-03, B-04, B-05, B-06, B-07, B-08, B-09, B-10, B-11, B-12, B-13, B-14, B-15, B-16, C-01, C-02, C-03, C-04, C-05, C-06, C-07, C-08, C-09, C-10, C-11, C-12, D-01, D-02, D-03, D-04, D-05, D-06, Pg-04, 97, 98, 116, 125, 129, 133, 135, 157a, 157b, 157c, 157d, 157e, 157f, 162, 163, 235a, 235b, 235c, 236a, 236b, 236c, 238a, 238b, 238c, 255, 256, 258a, 258b, 258c, 260, 262, 267, 271, 272, 274, 276, 278, 282, 284, 286, 287, 306, 309, 314, 318, or 325, as illustrated below:

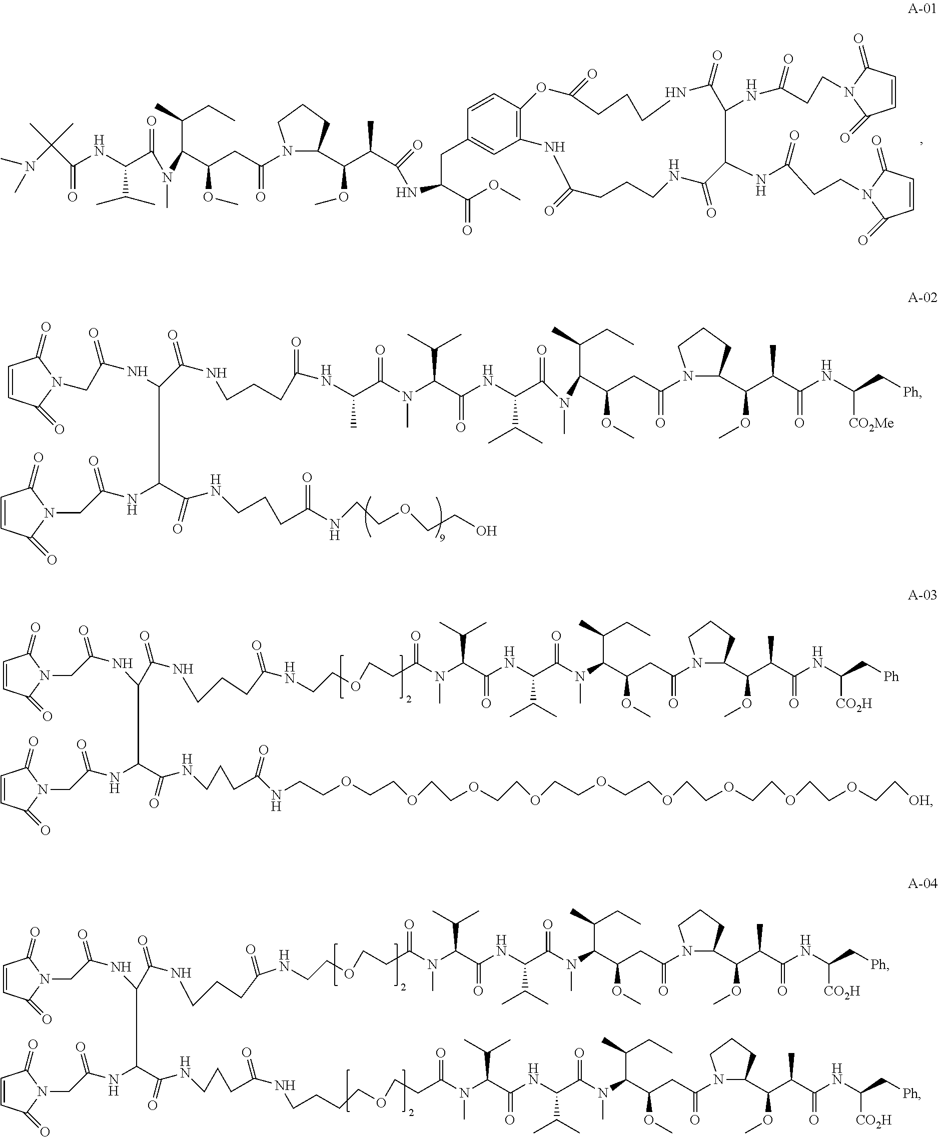

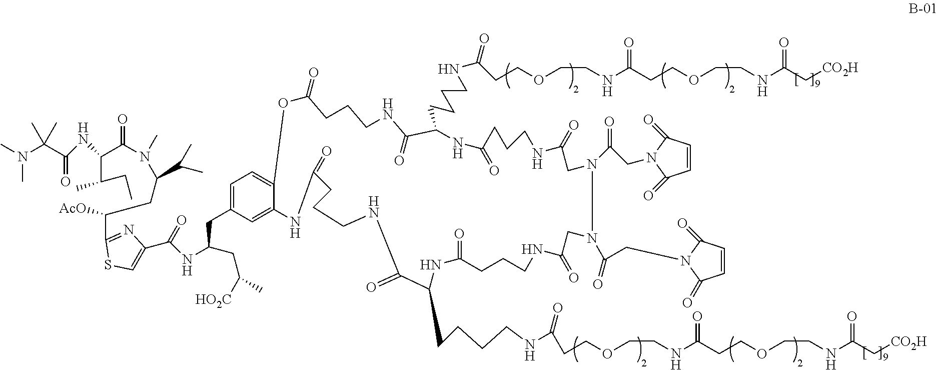

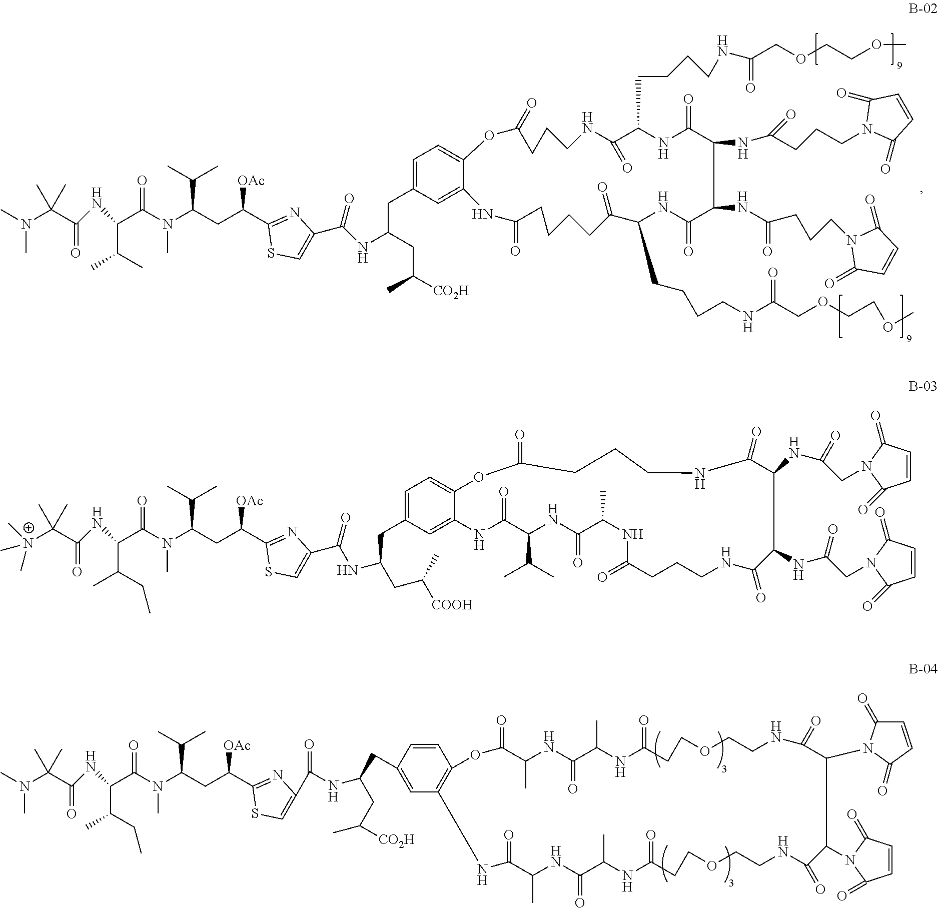

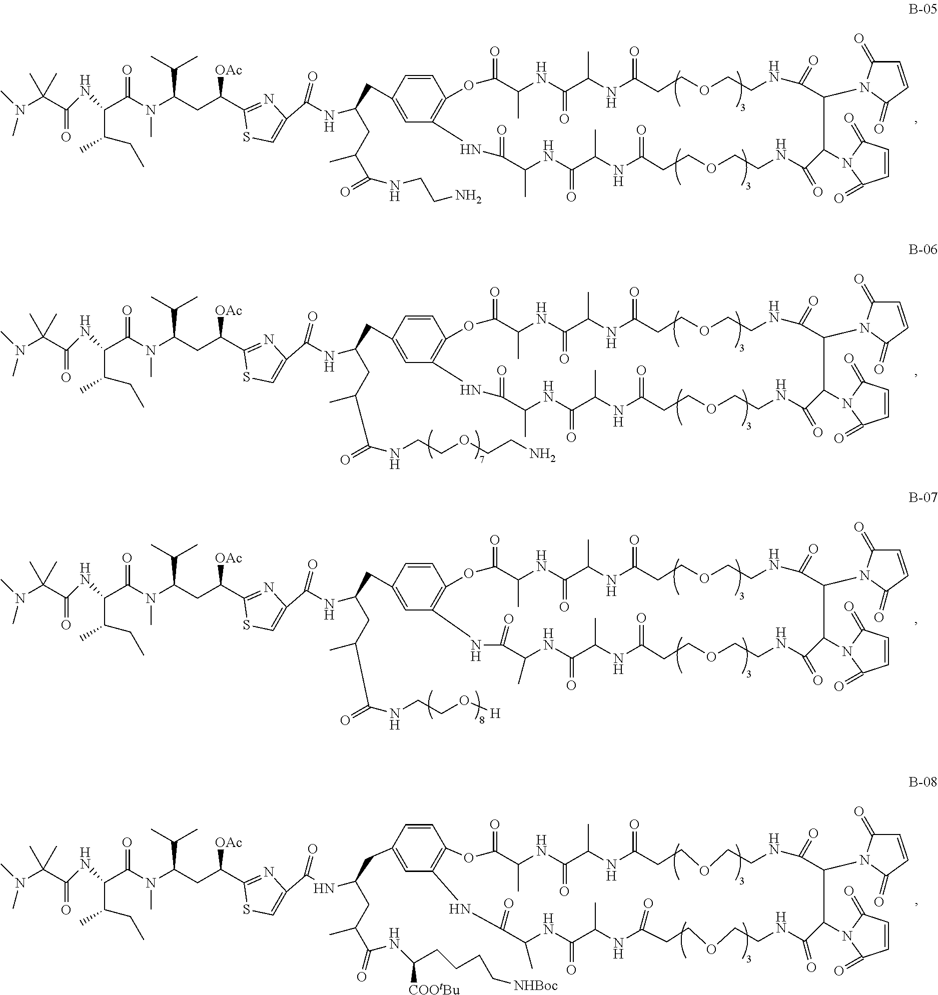

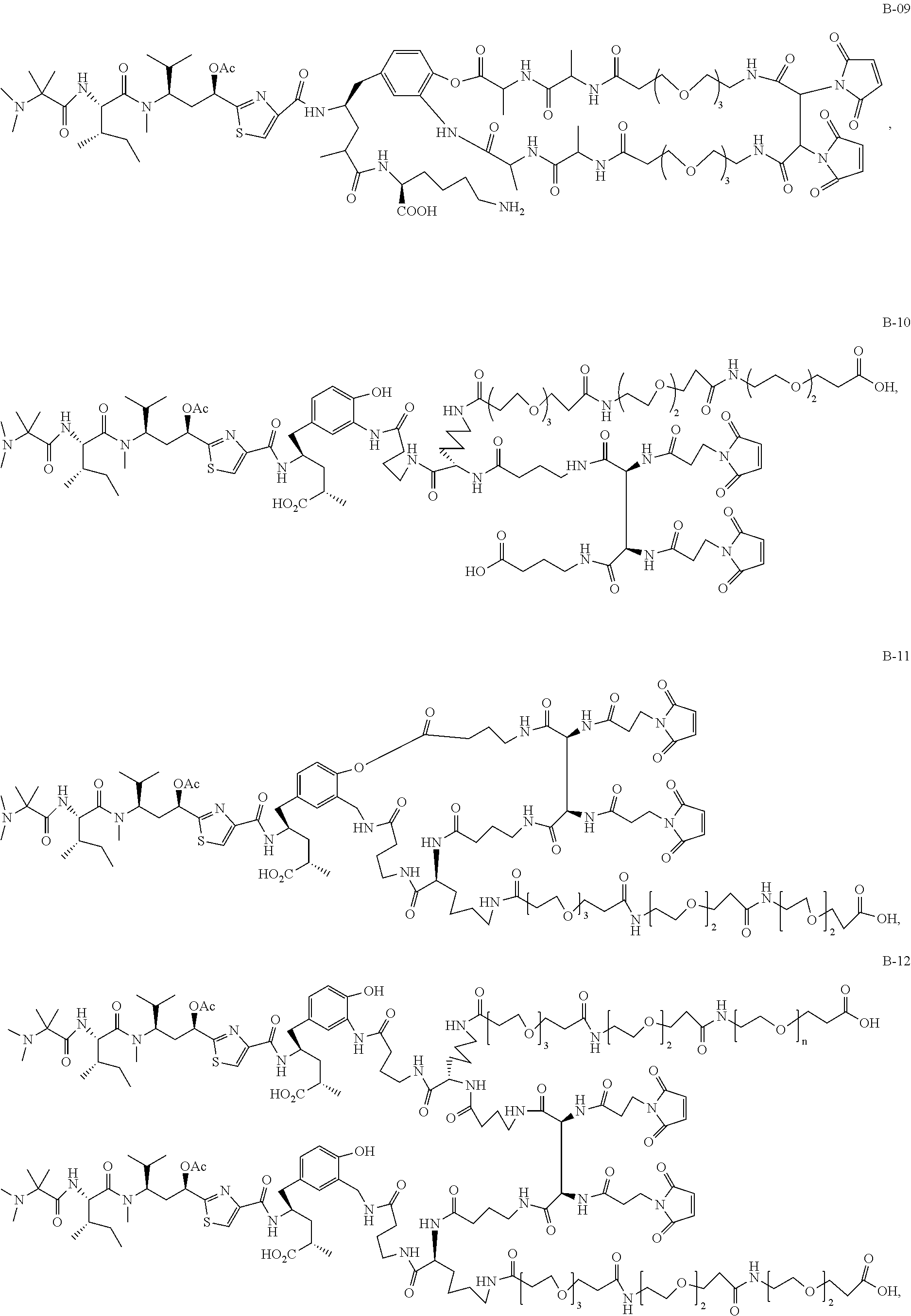

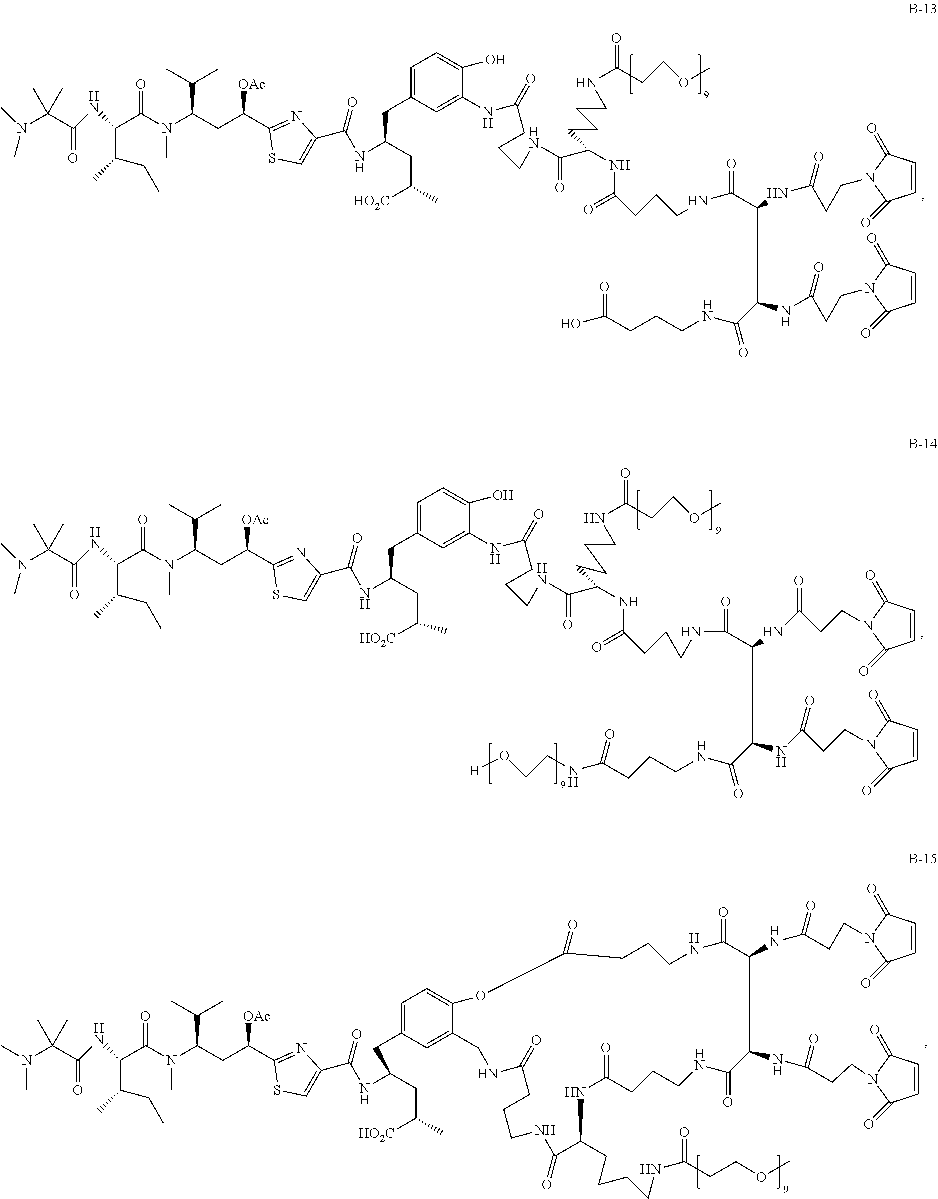

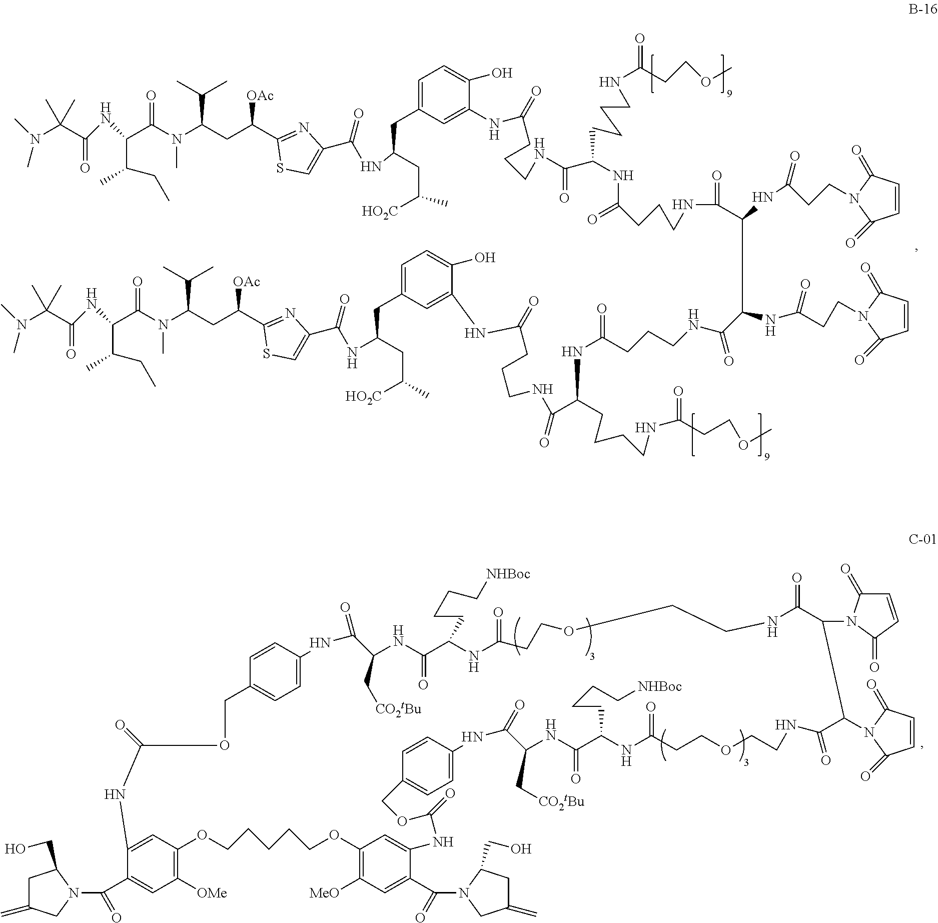

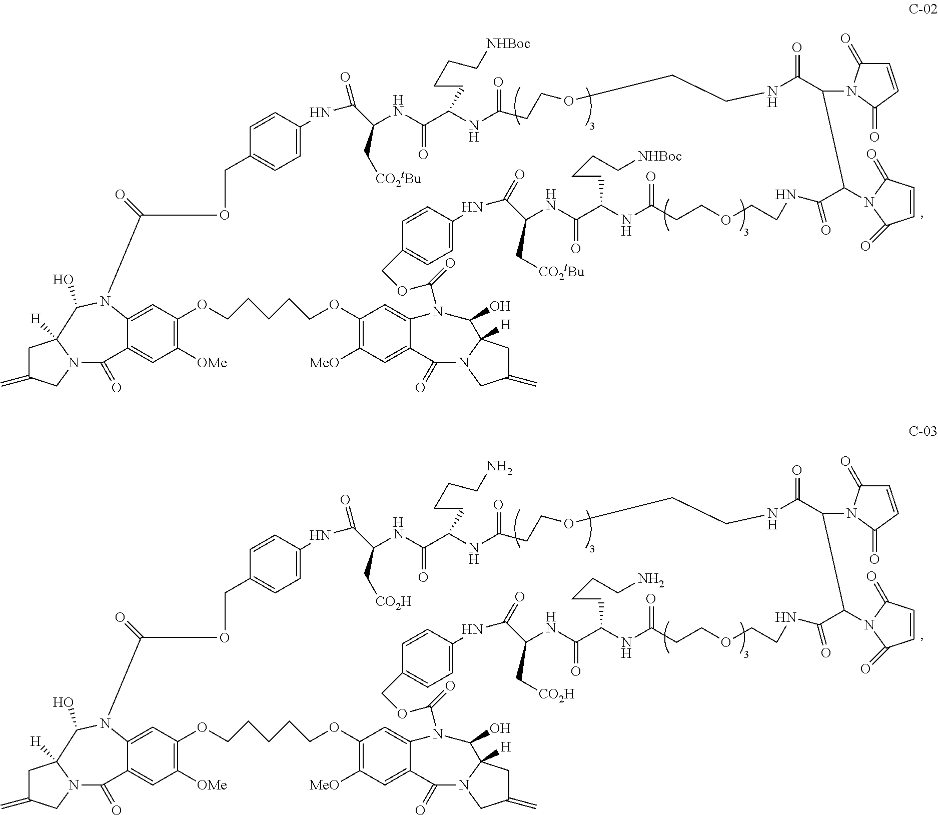

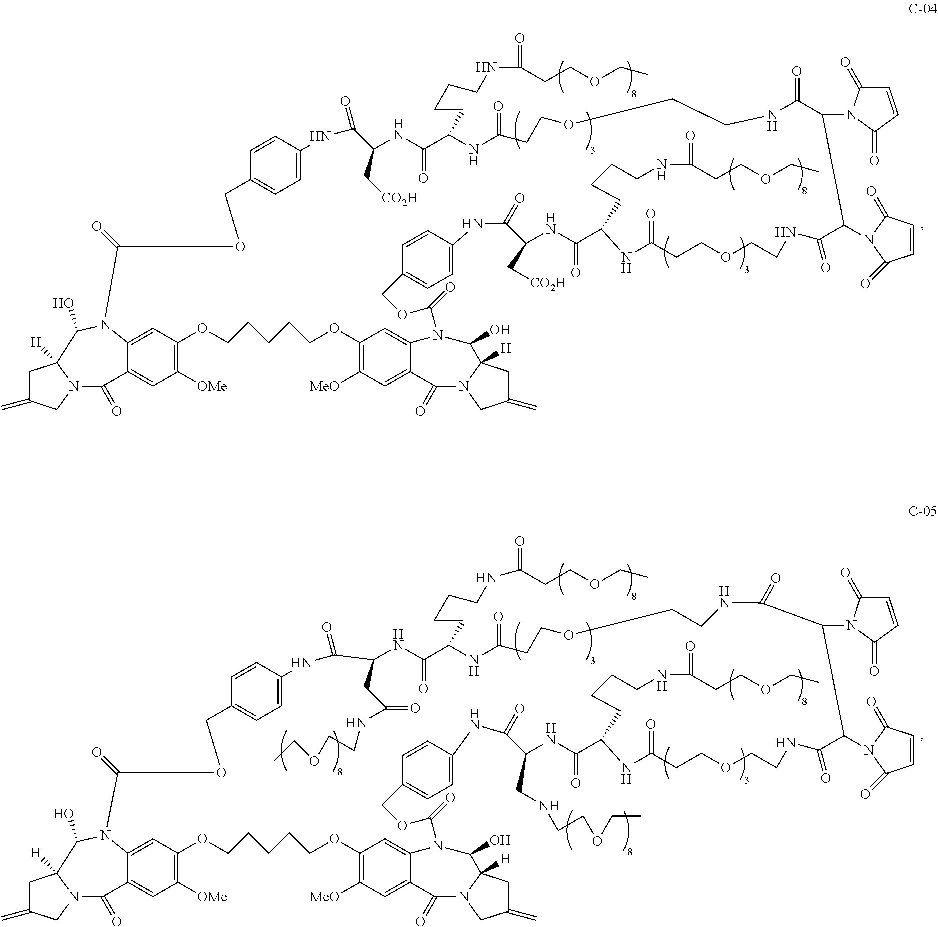

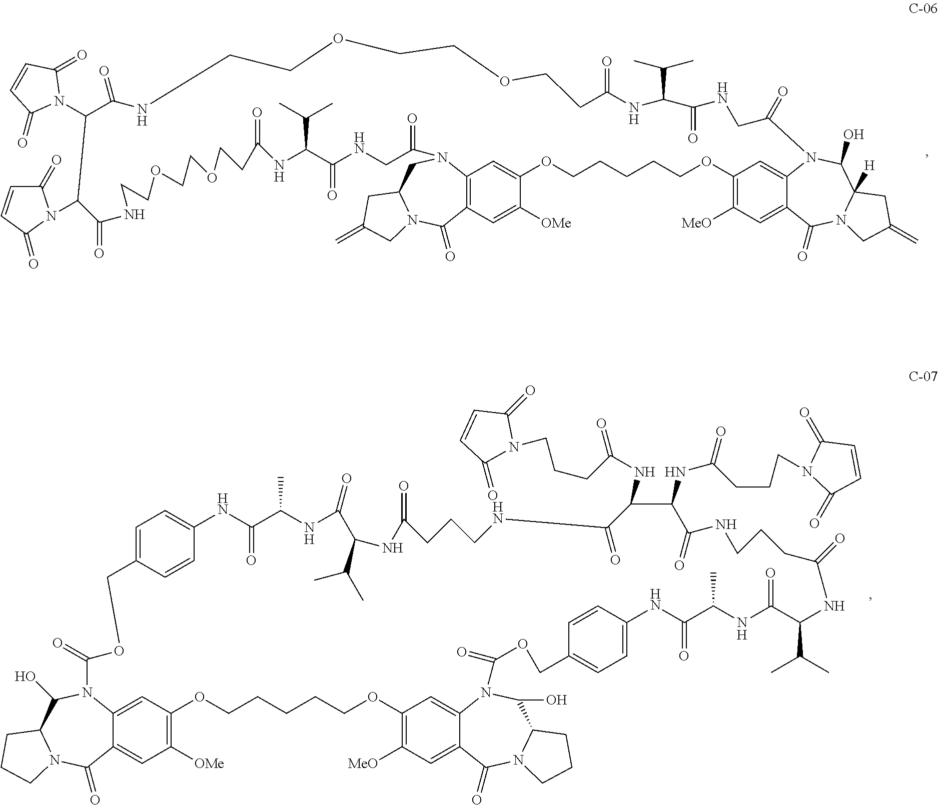

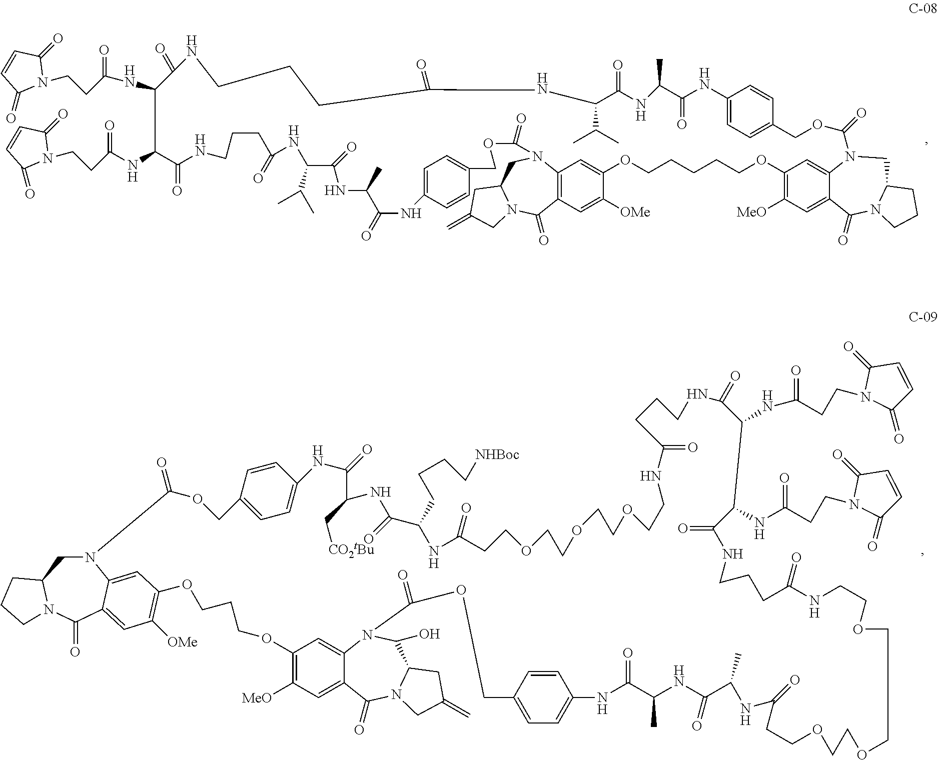

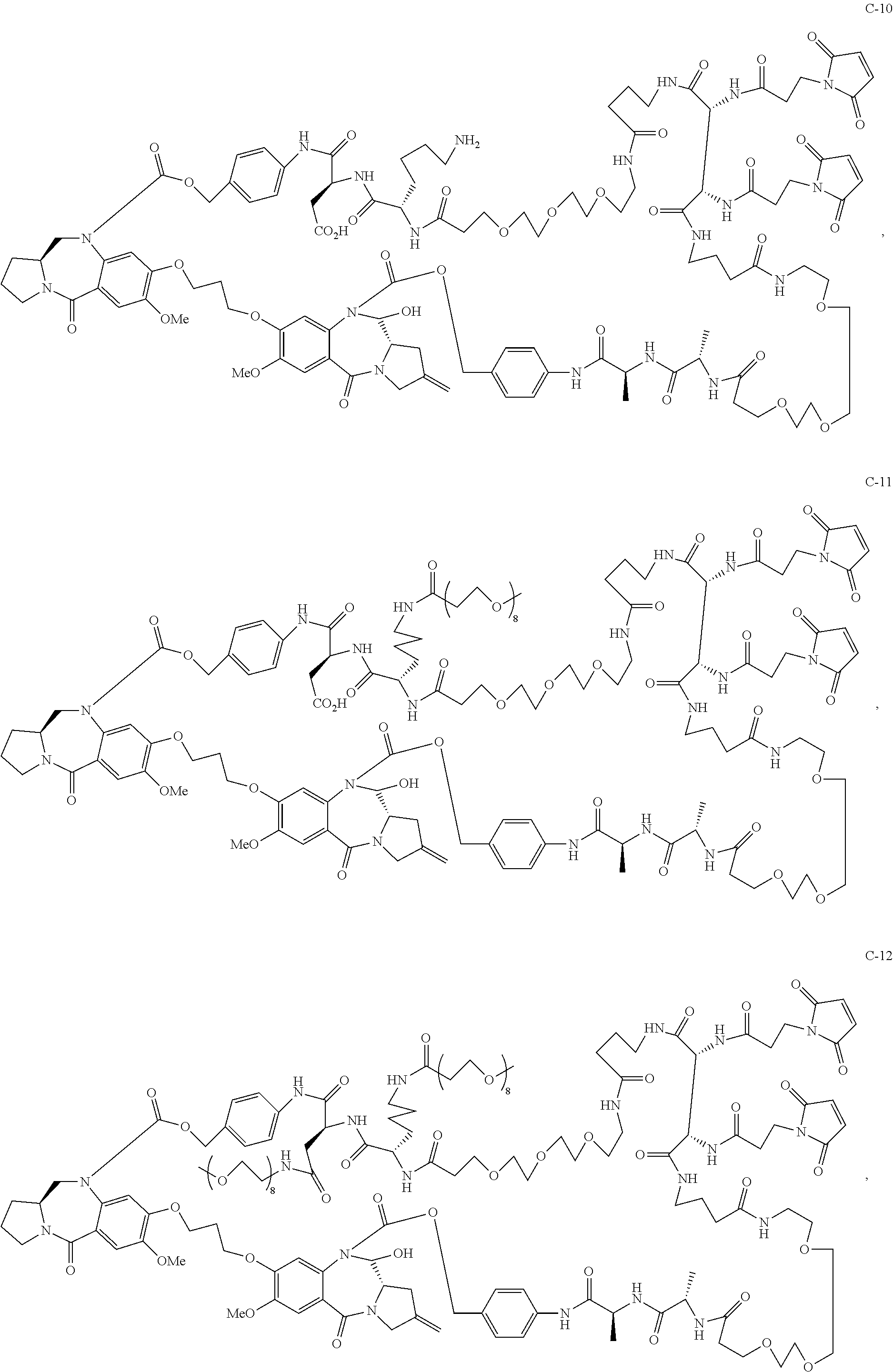

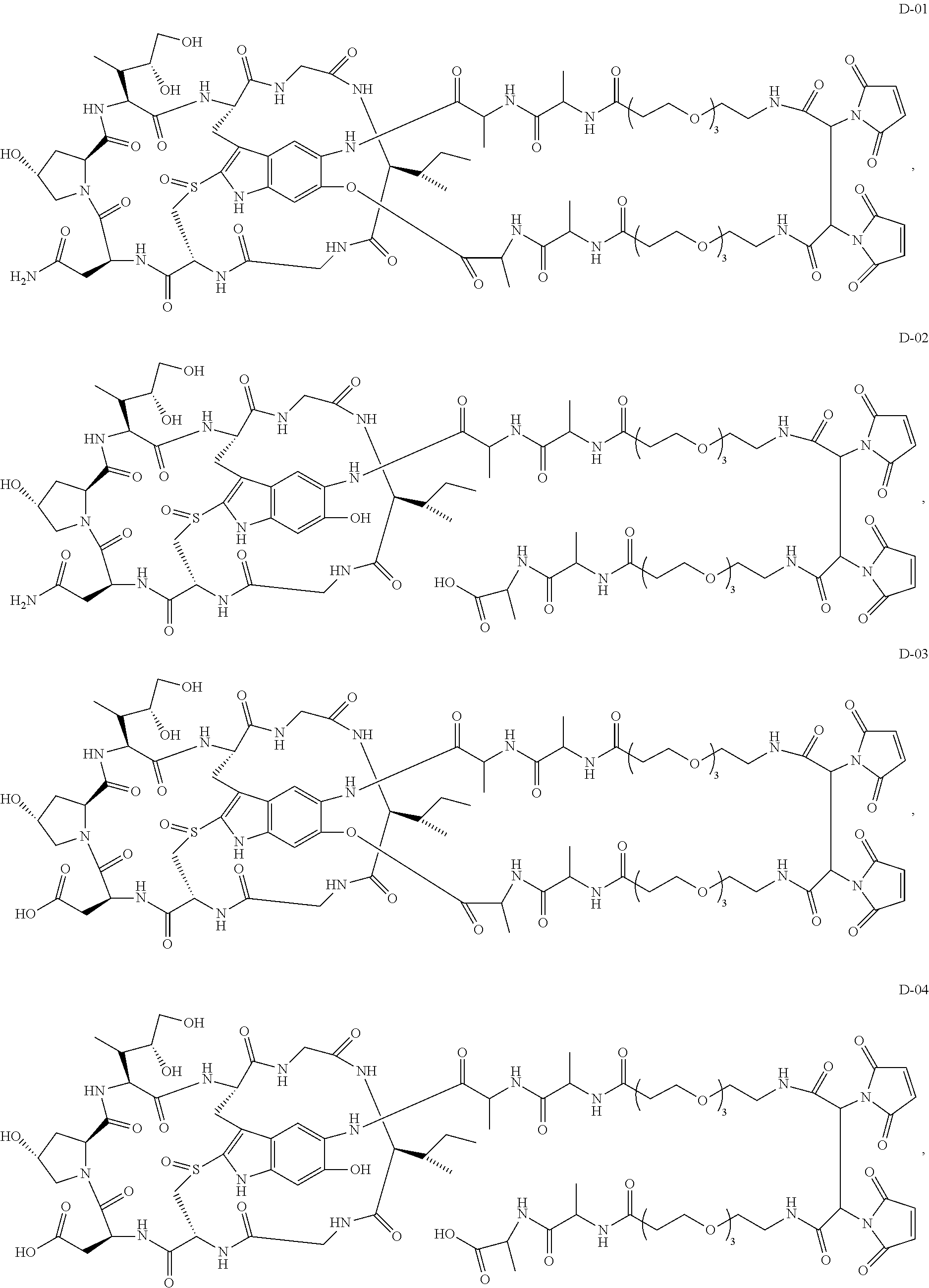

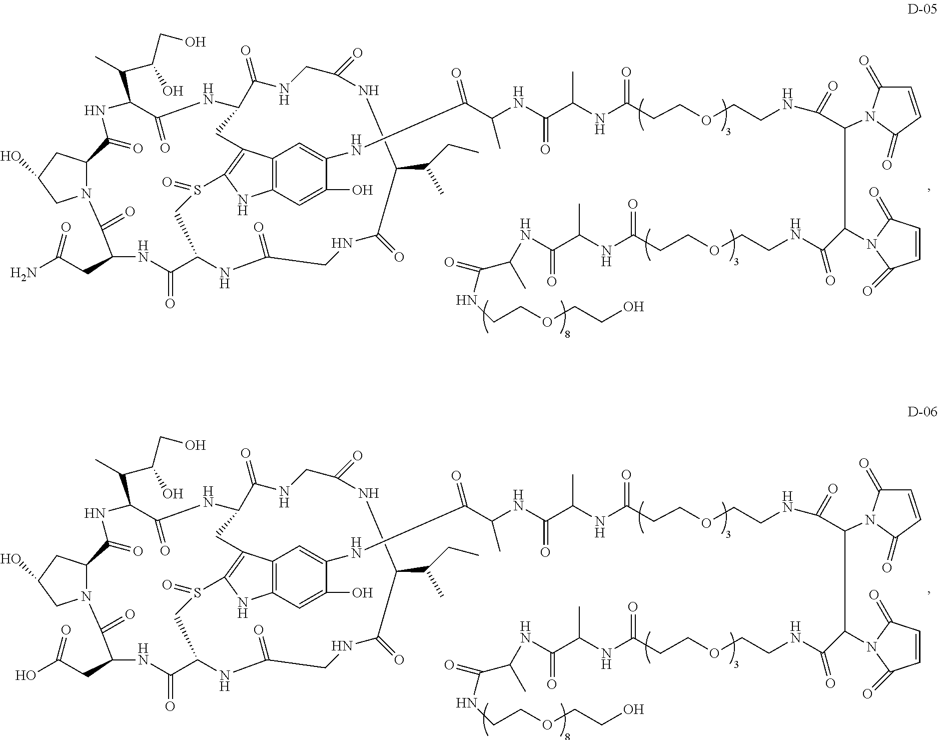

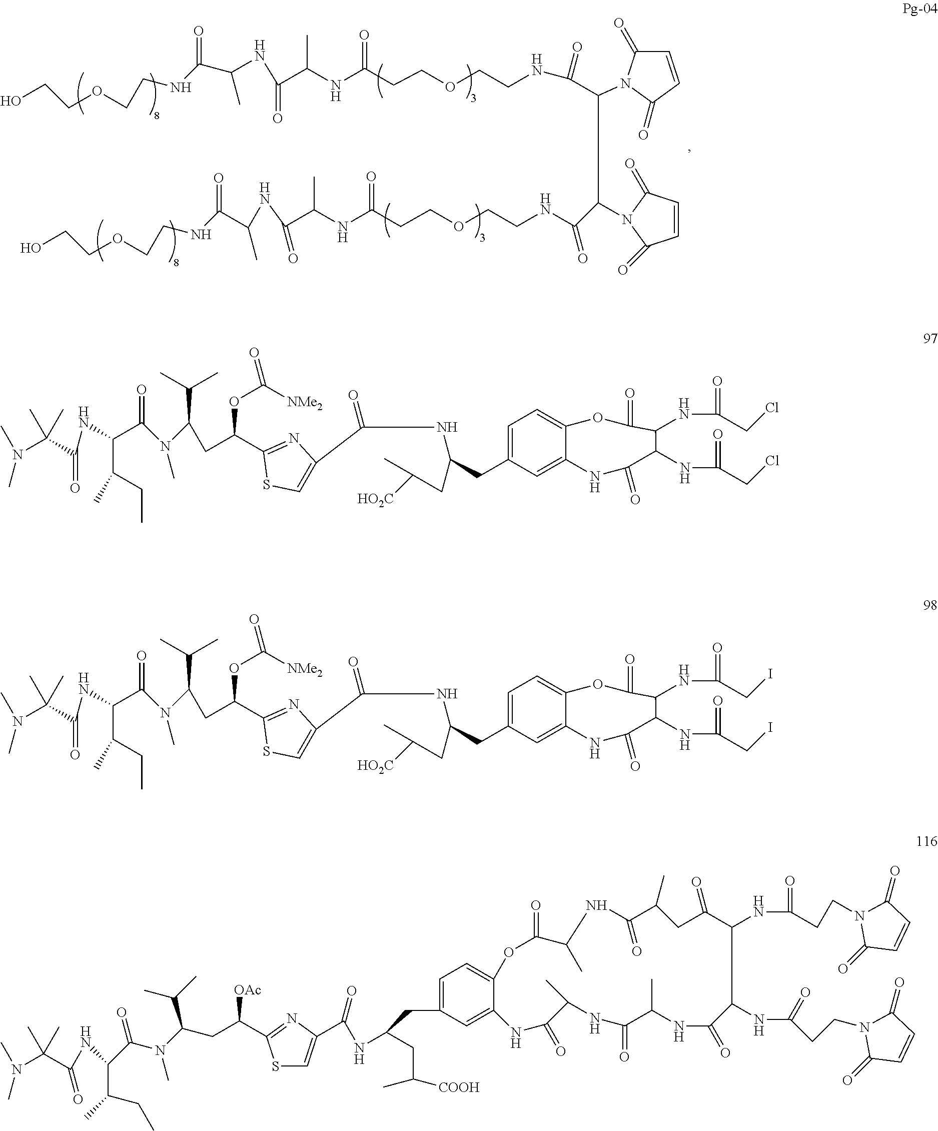

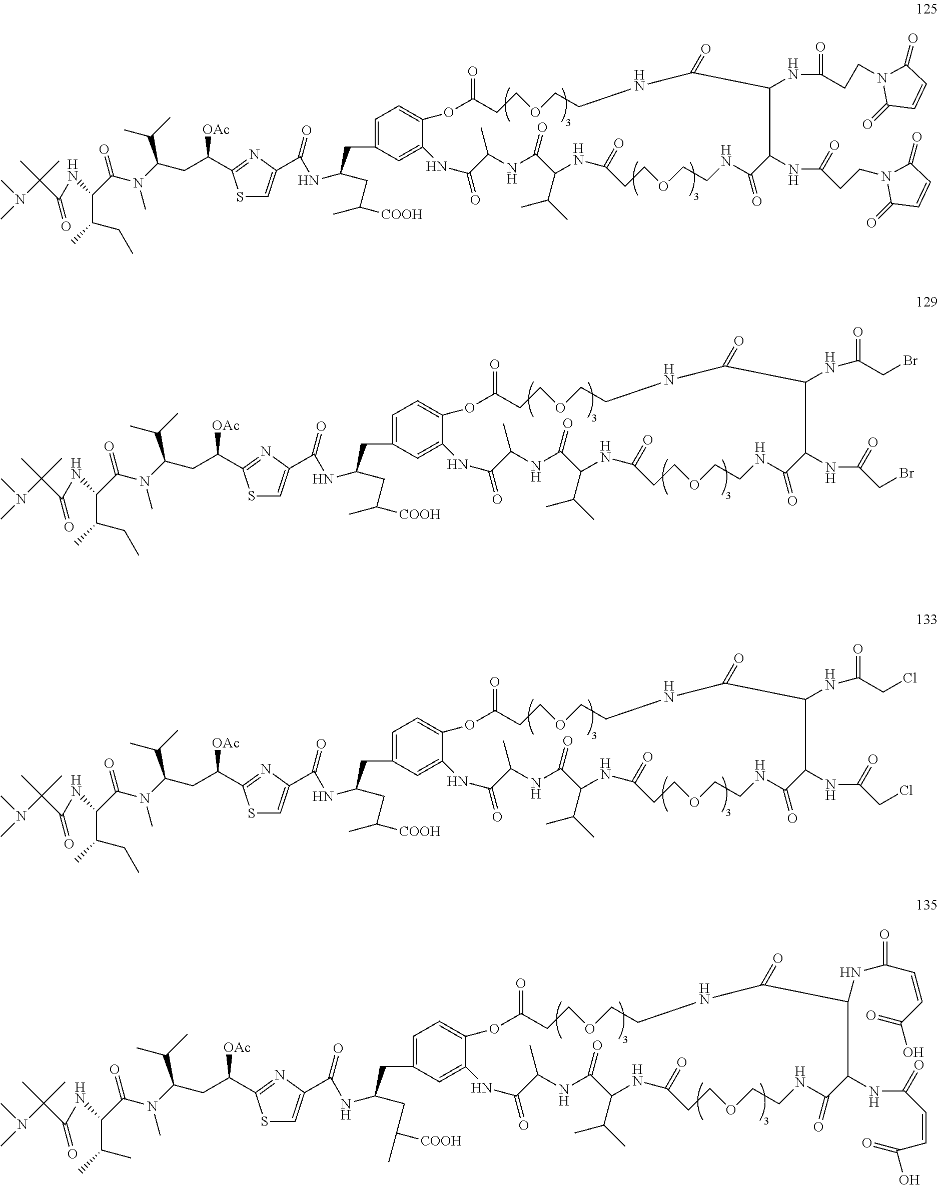

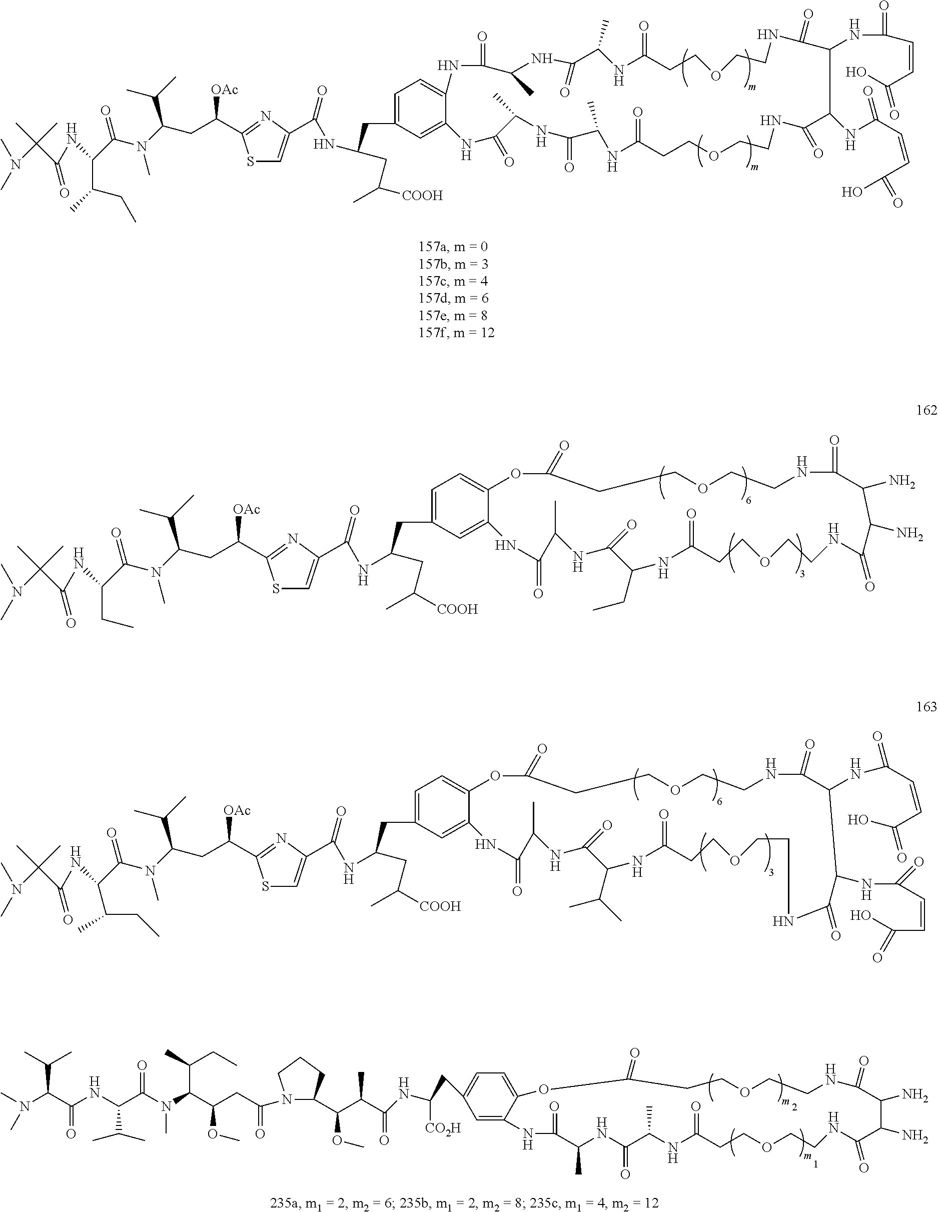

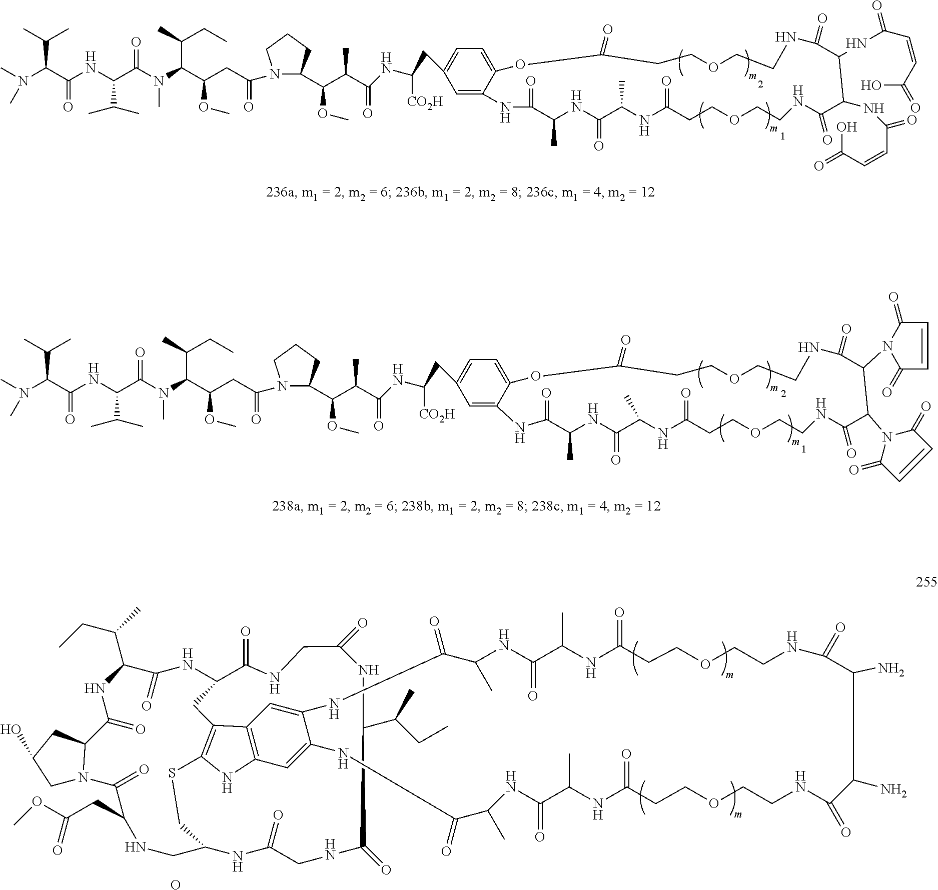

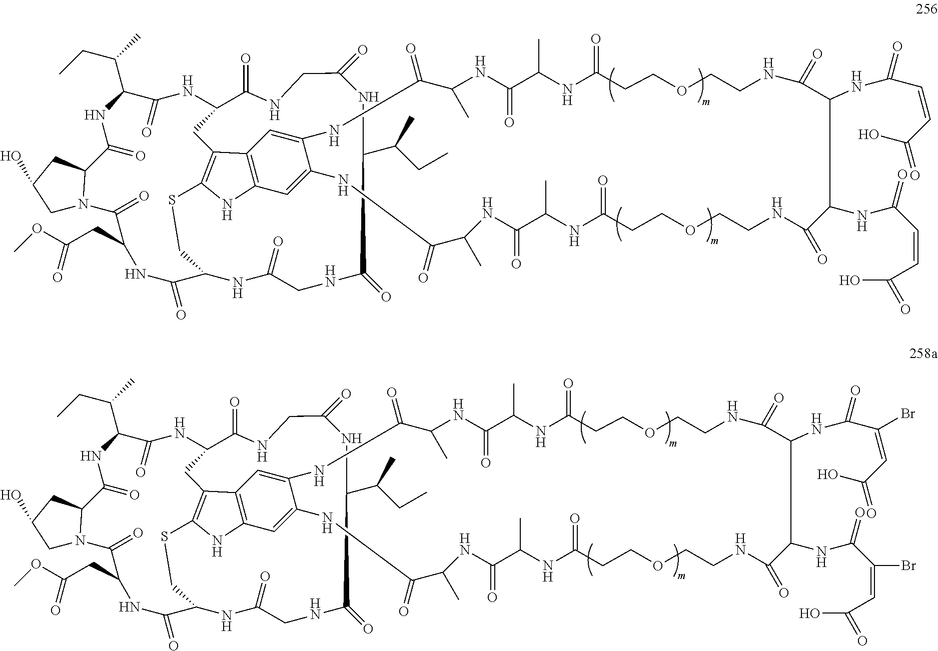

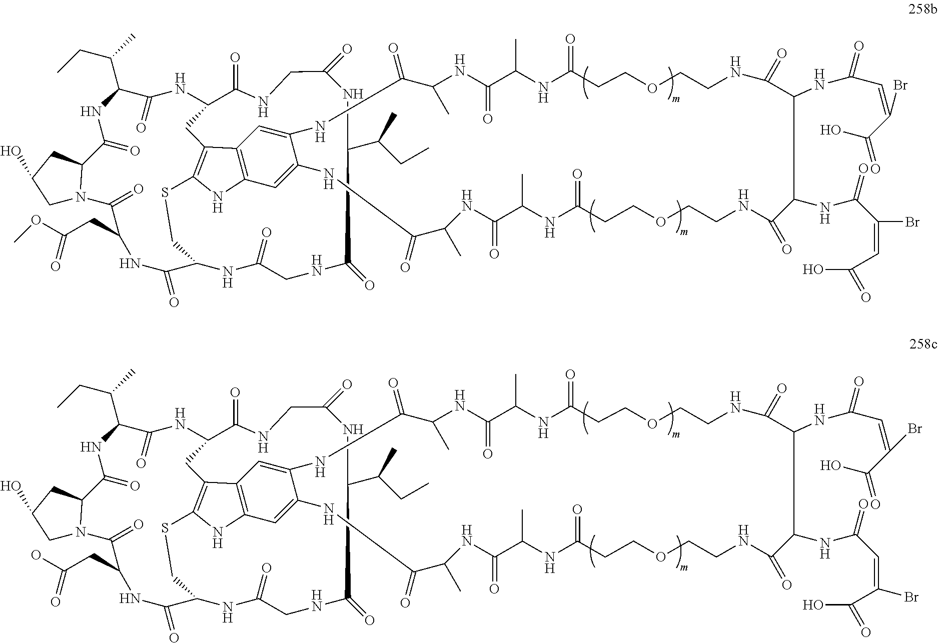

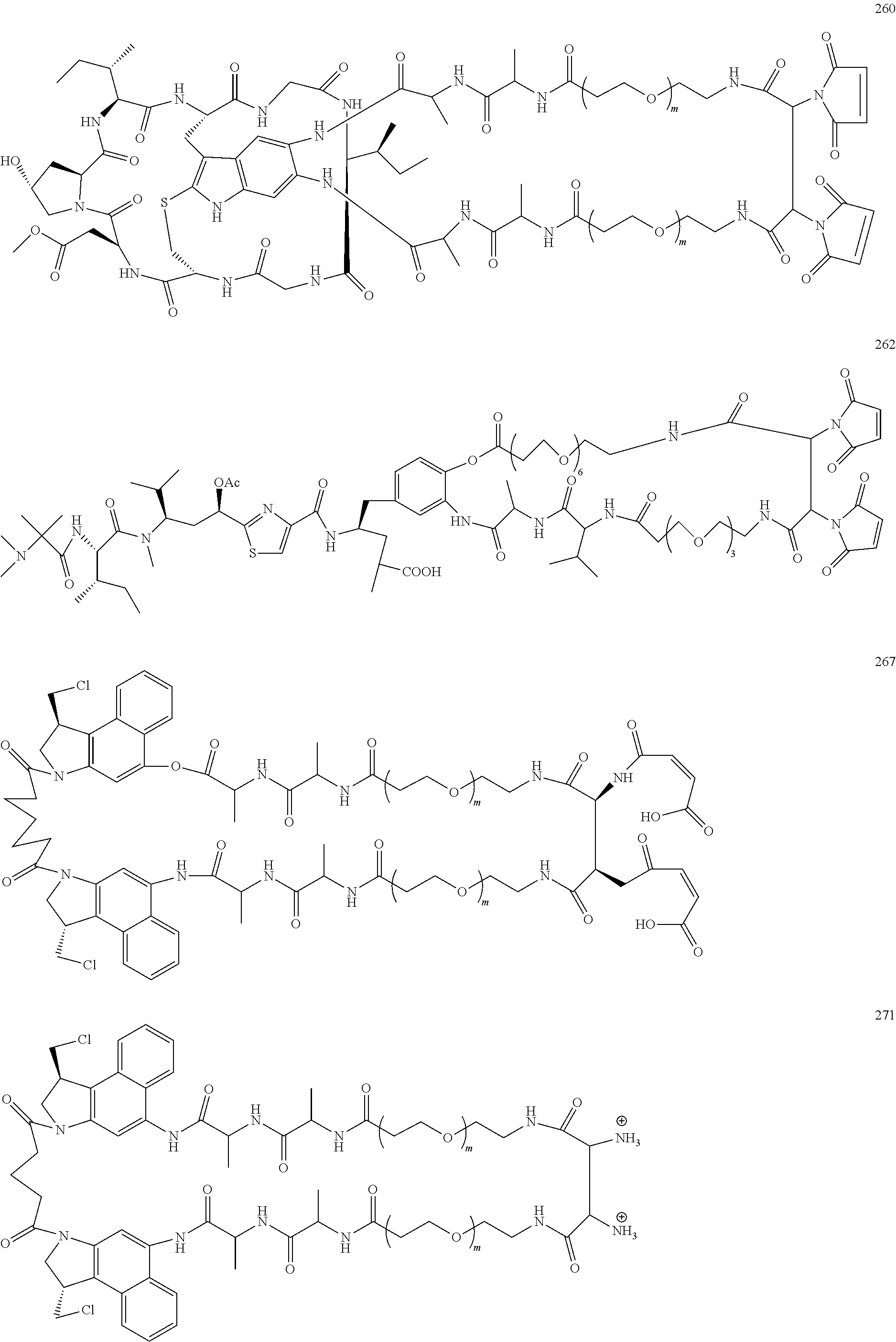

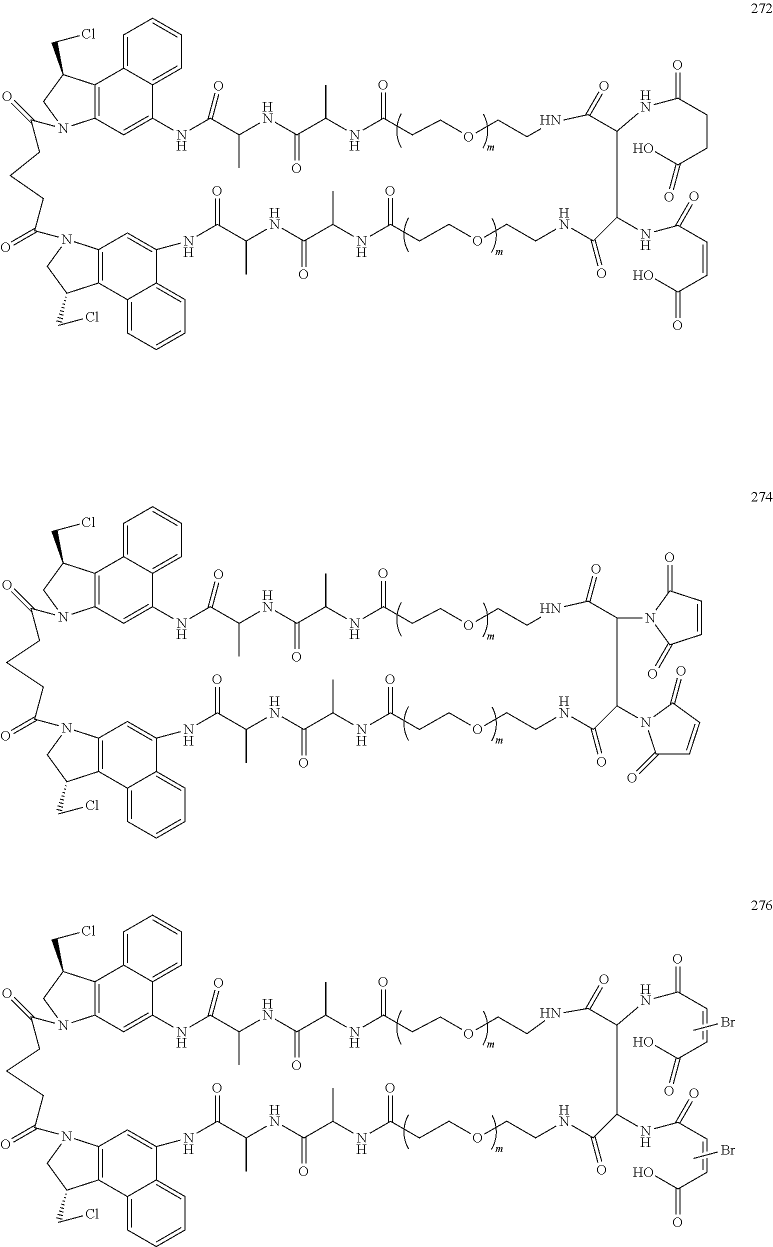

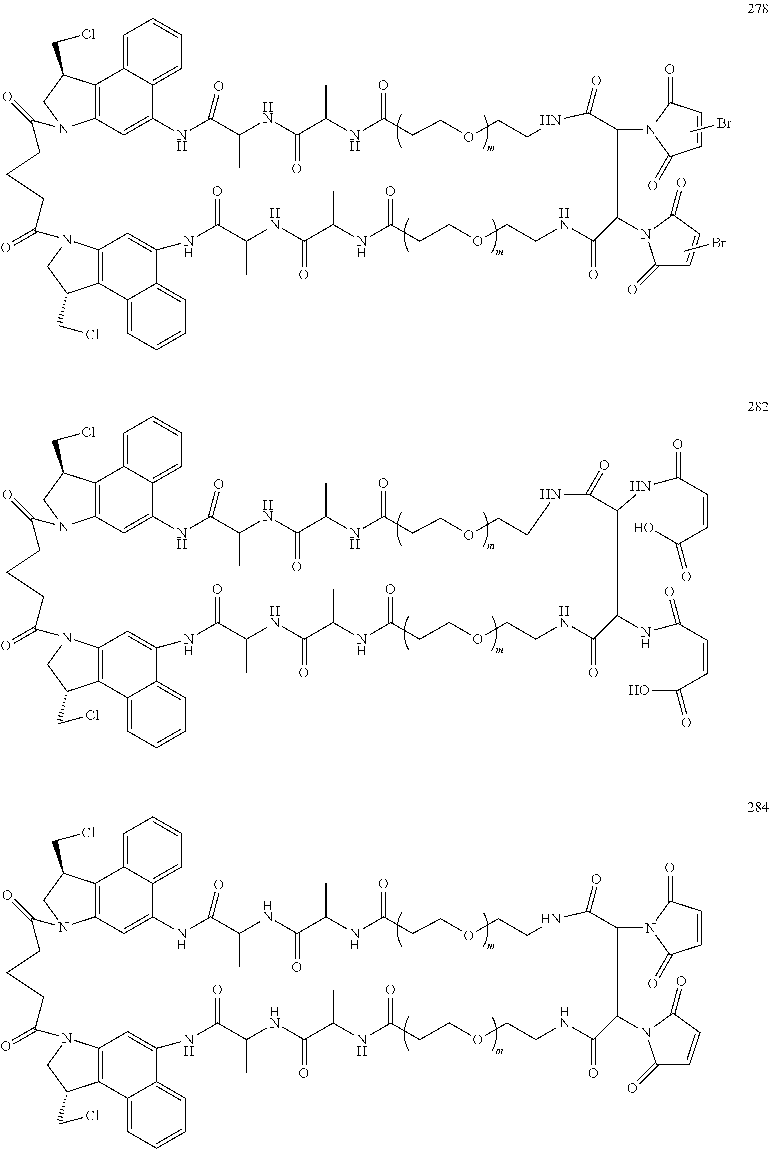

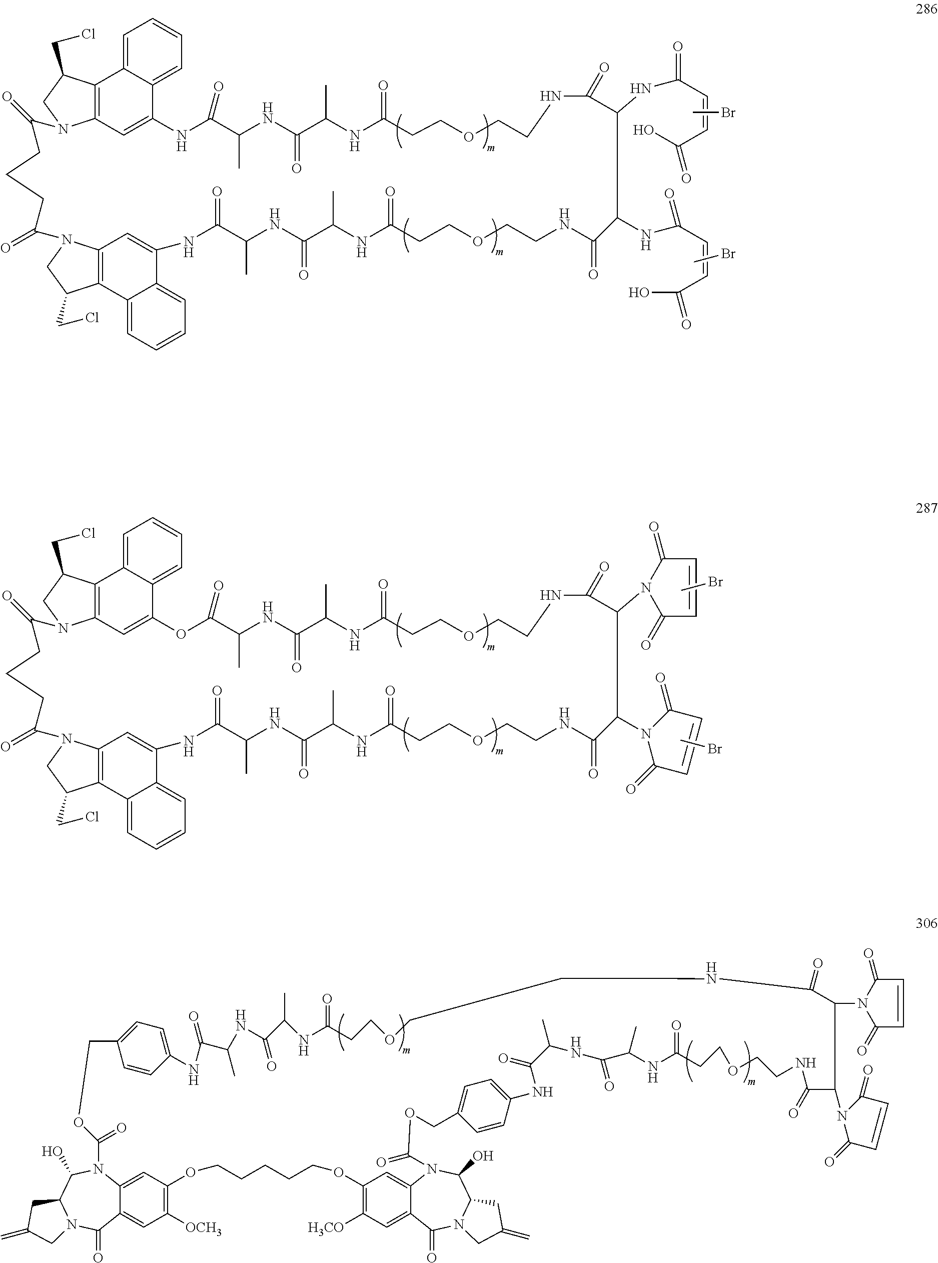

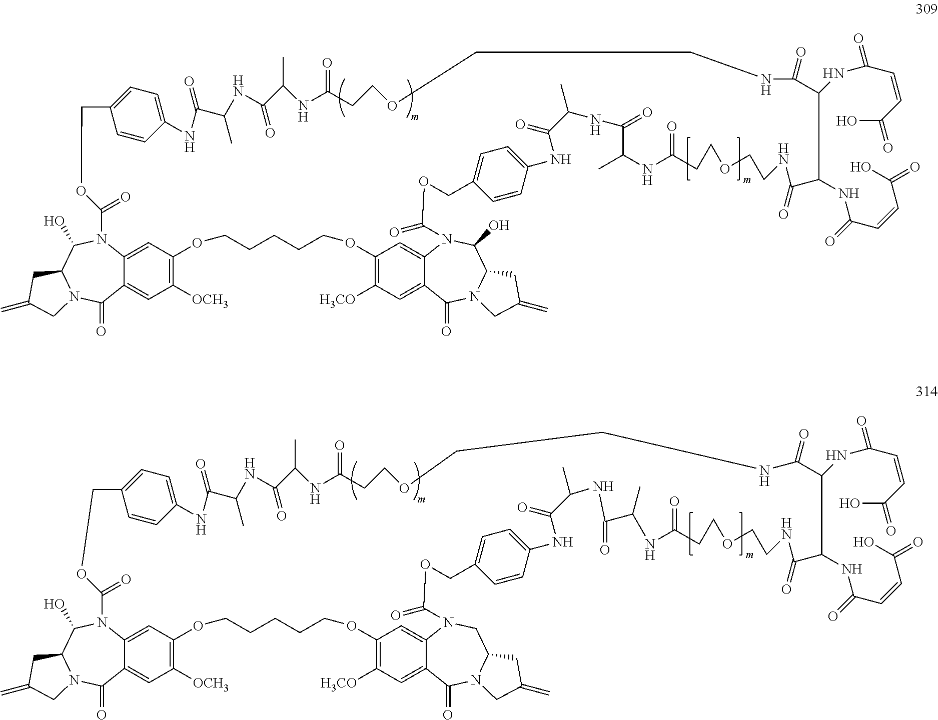

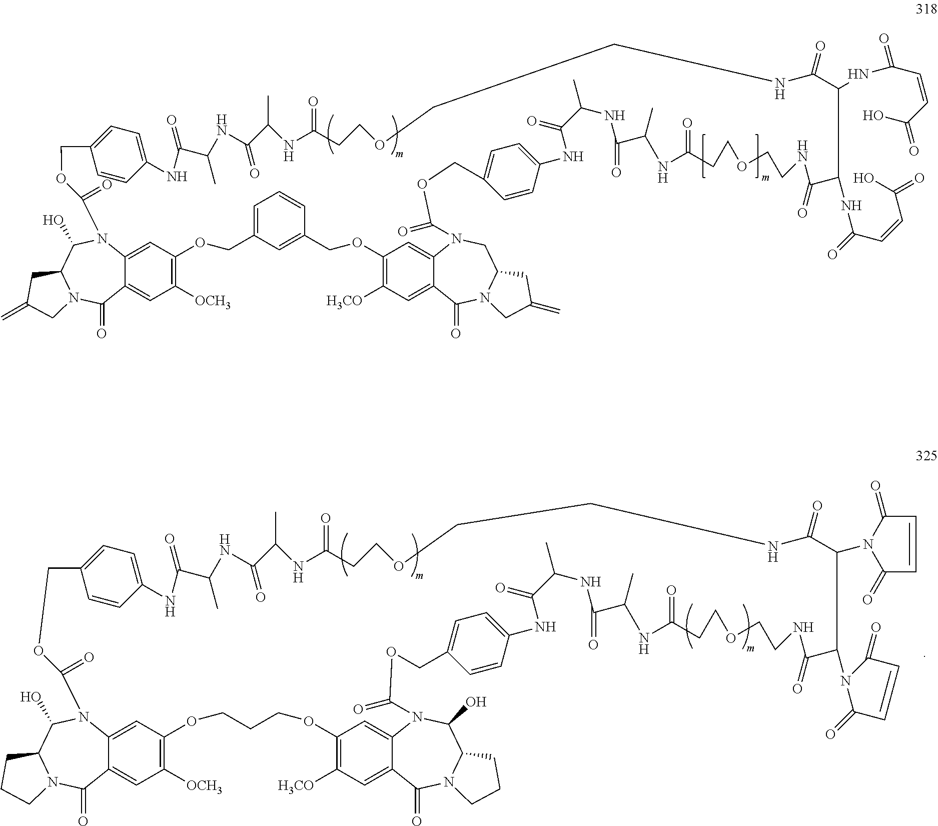

84. The conjugate compound of claim 48, having the Formula of Aa-01, Aa-02, Aa-03, Aa-04, Ba-01, Ba-02, Ba-03, Ba-04, Ba-05, Ba-06, Ba-07, Ba-08, Ba-09, Ba-10, Ba-11, Ba-12, Ba-13, Ba-14, Ba-15, Ba-16, Ca-02, Ca-03, Ca-04, Ca-05, Ca-06, Ca-07, Ca-08, Ca-09, Ca-10, Ca-11, Ca-12, Da-01, Da-02, Da-03, Da-04, Da-05 or Da-06, 99, 117, 126, 130, 136, 158a, 158b, 158c, 158d, 158e, 158f, 164, 237a, 237b, 237c, 239a, 239b, 239c, 257, 259, 261, 263, 268, 273, 275, 277, 279, 283, 285, 288, 307, 310, 315, 319, or 326, as shown in the following structures: 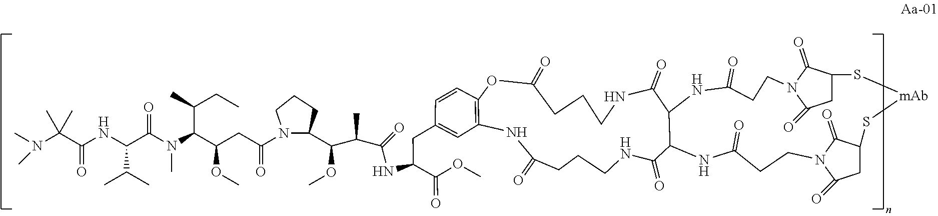 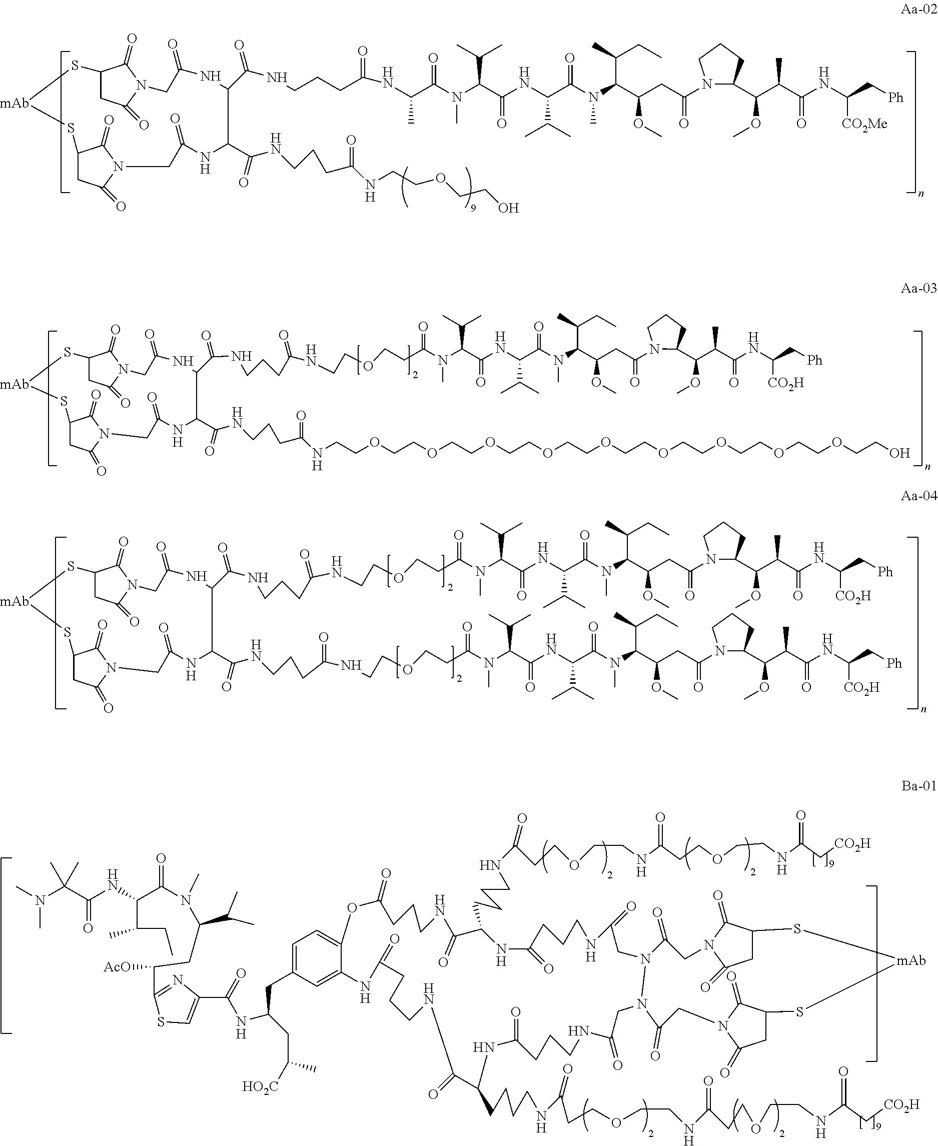 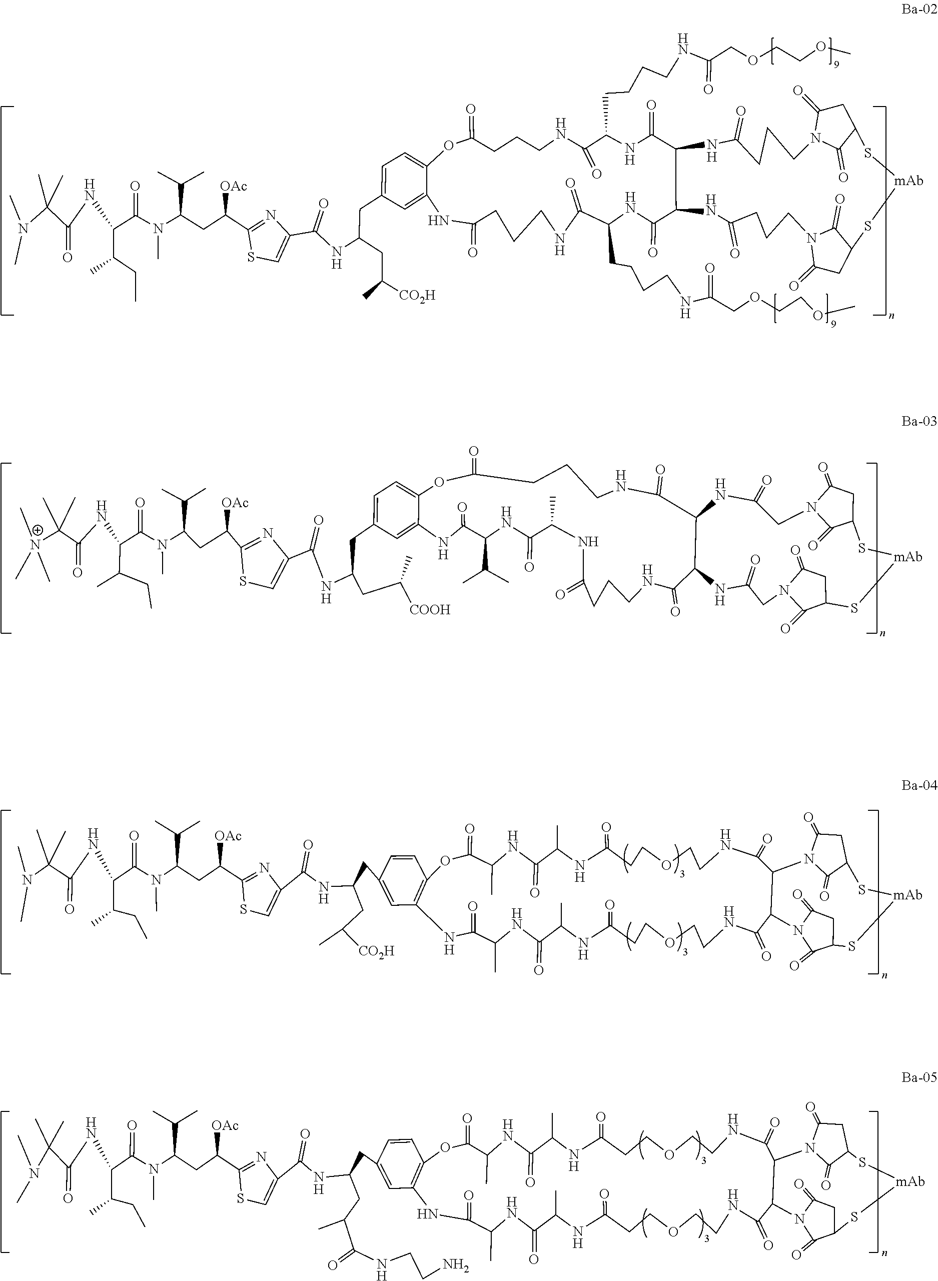 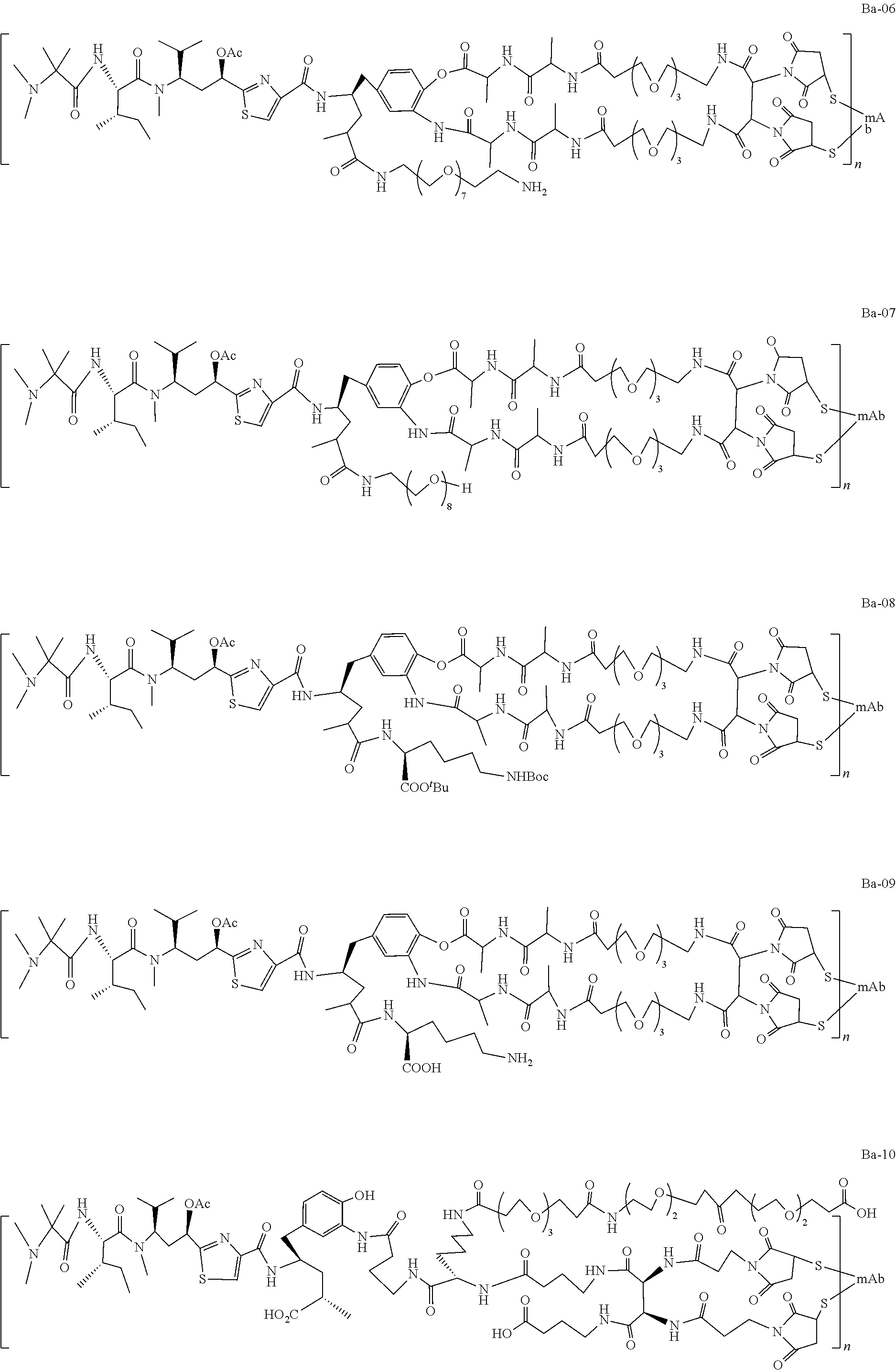 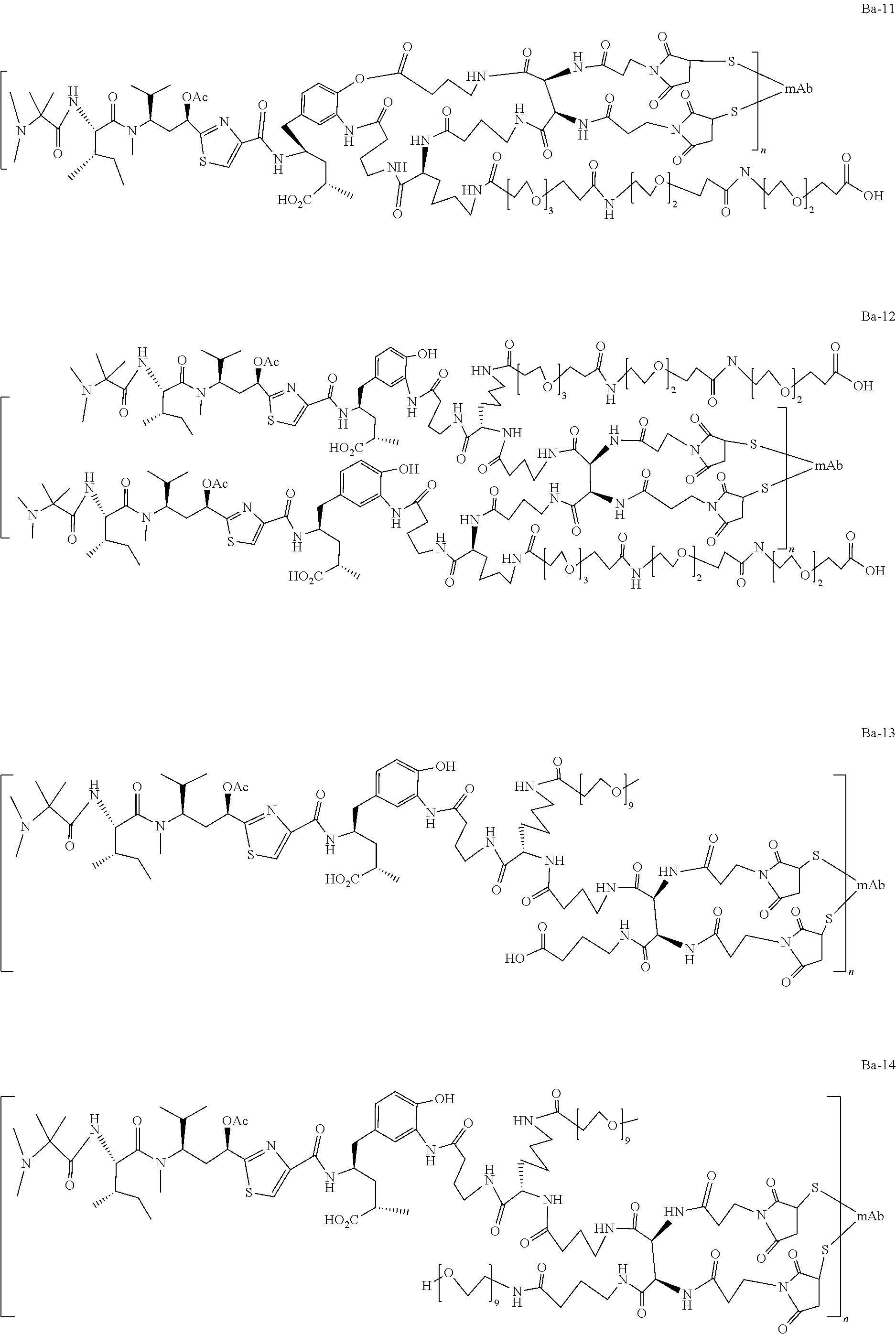 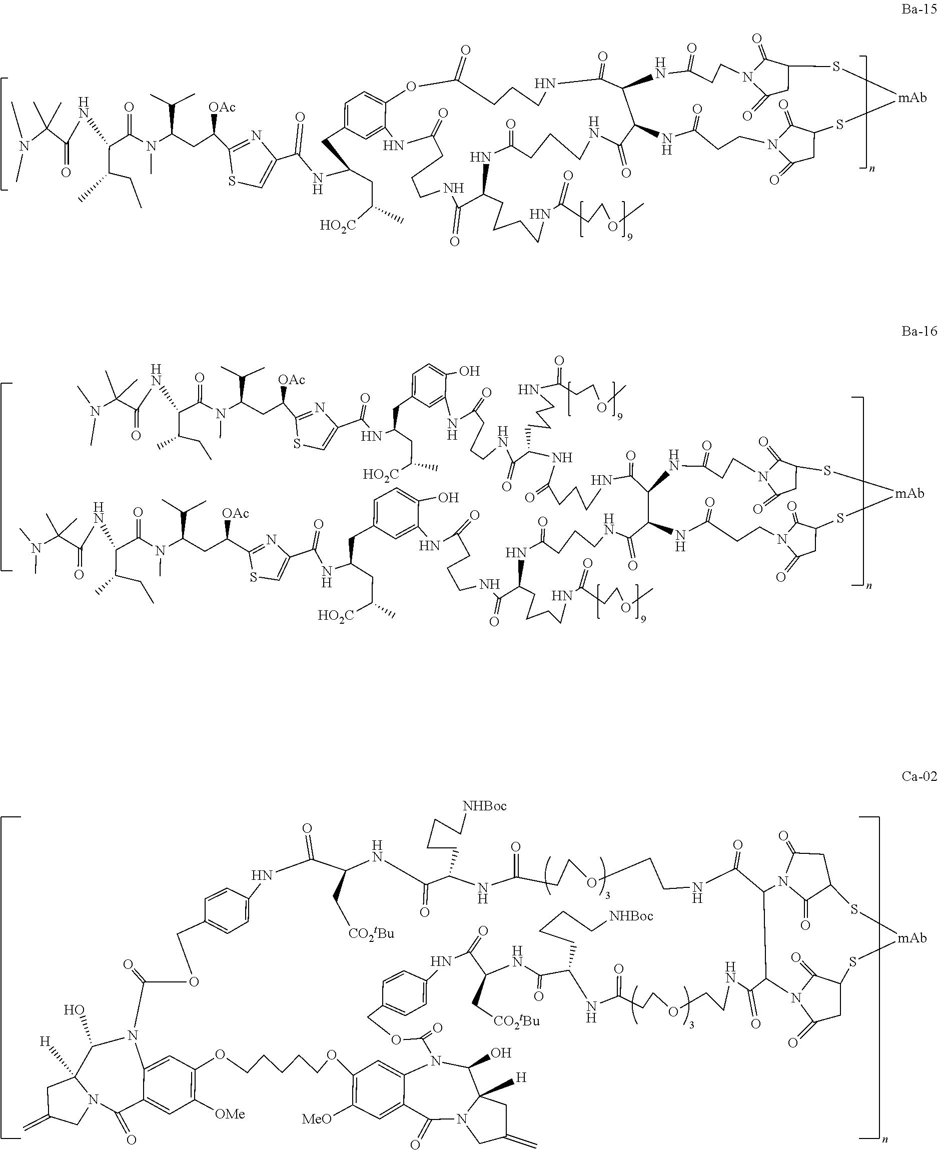 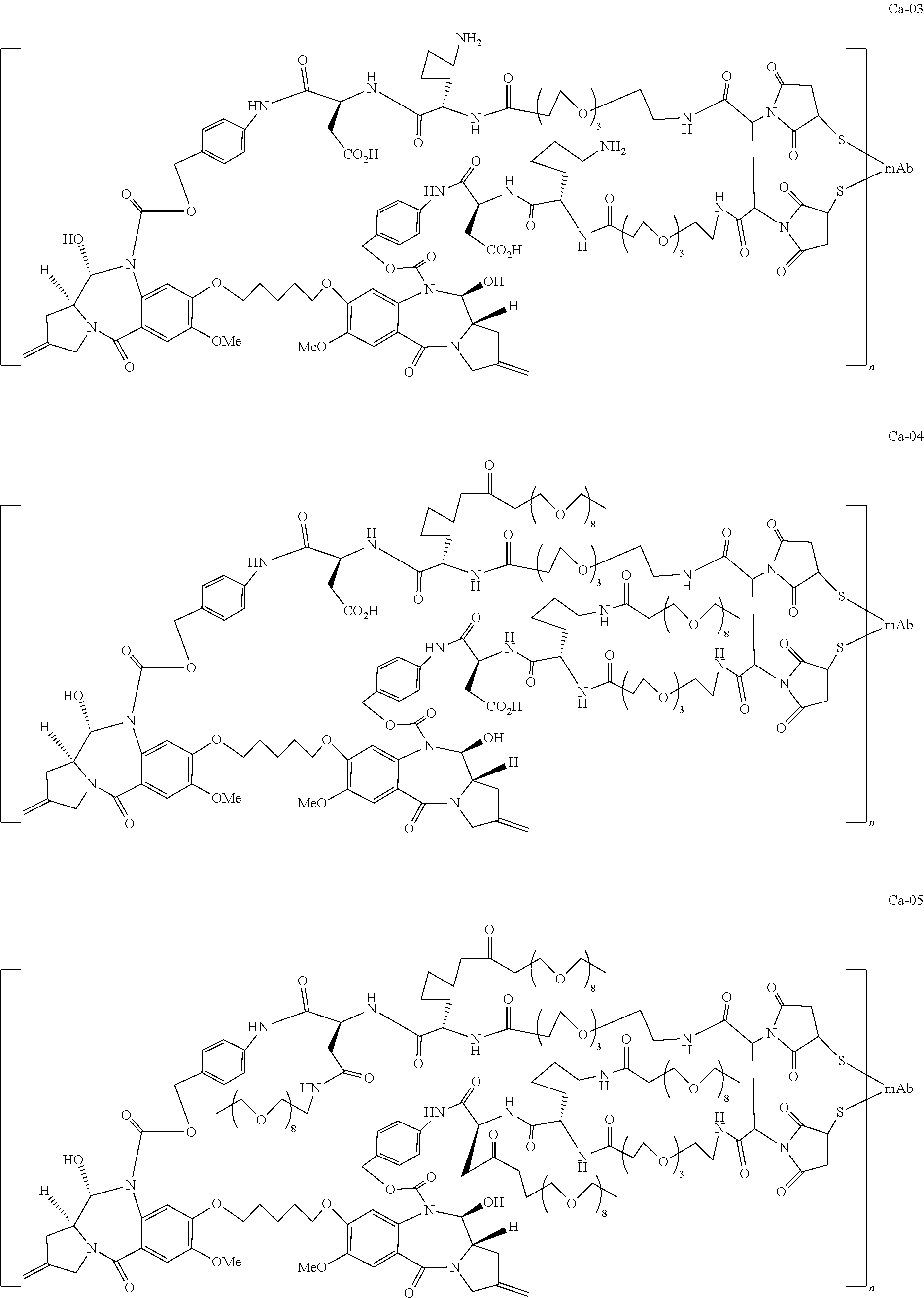 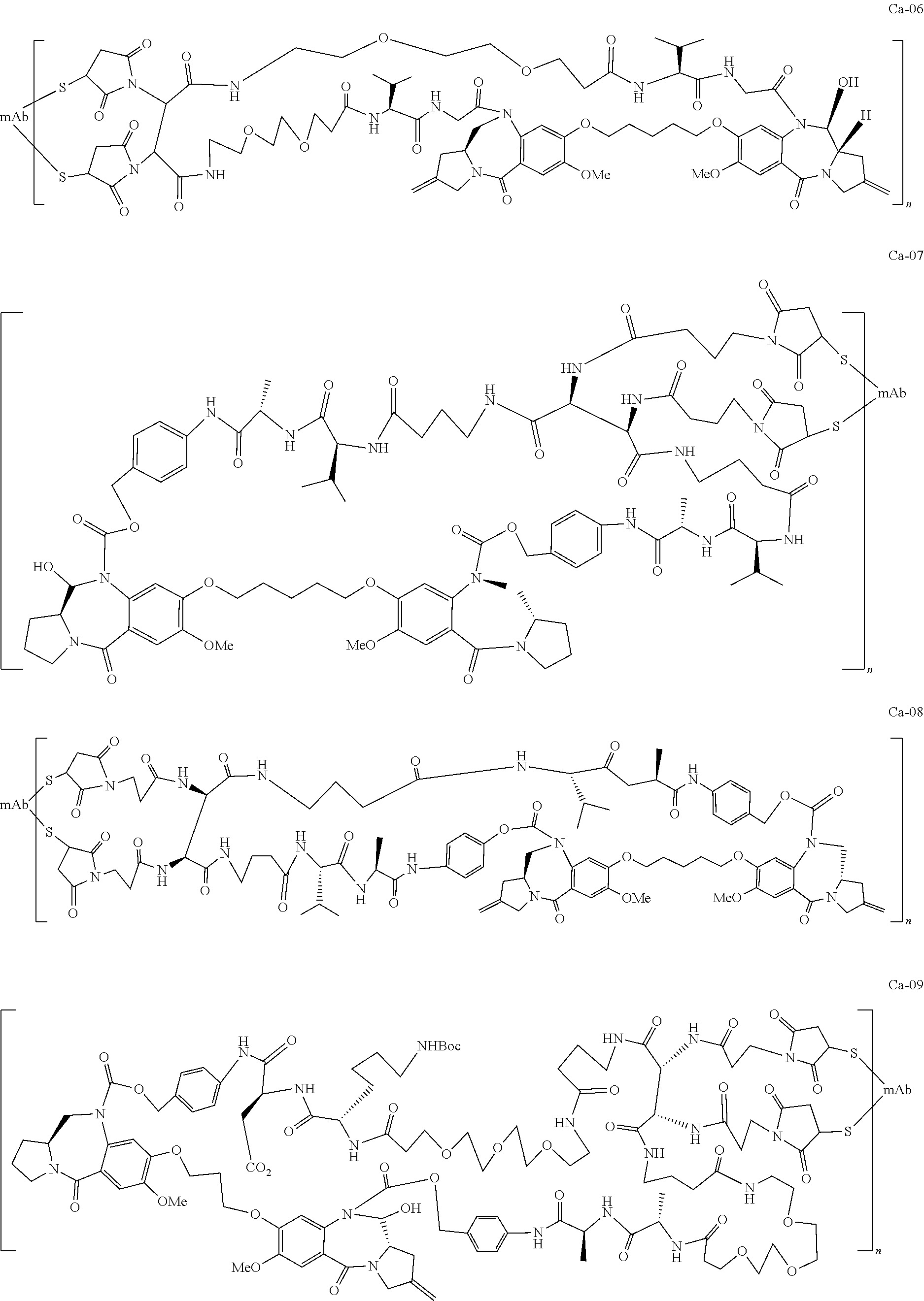 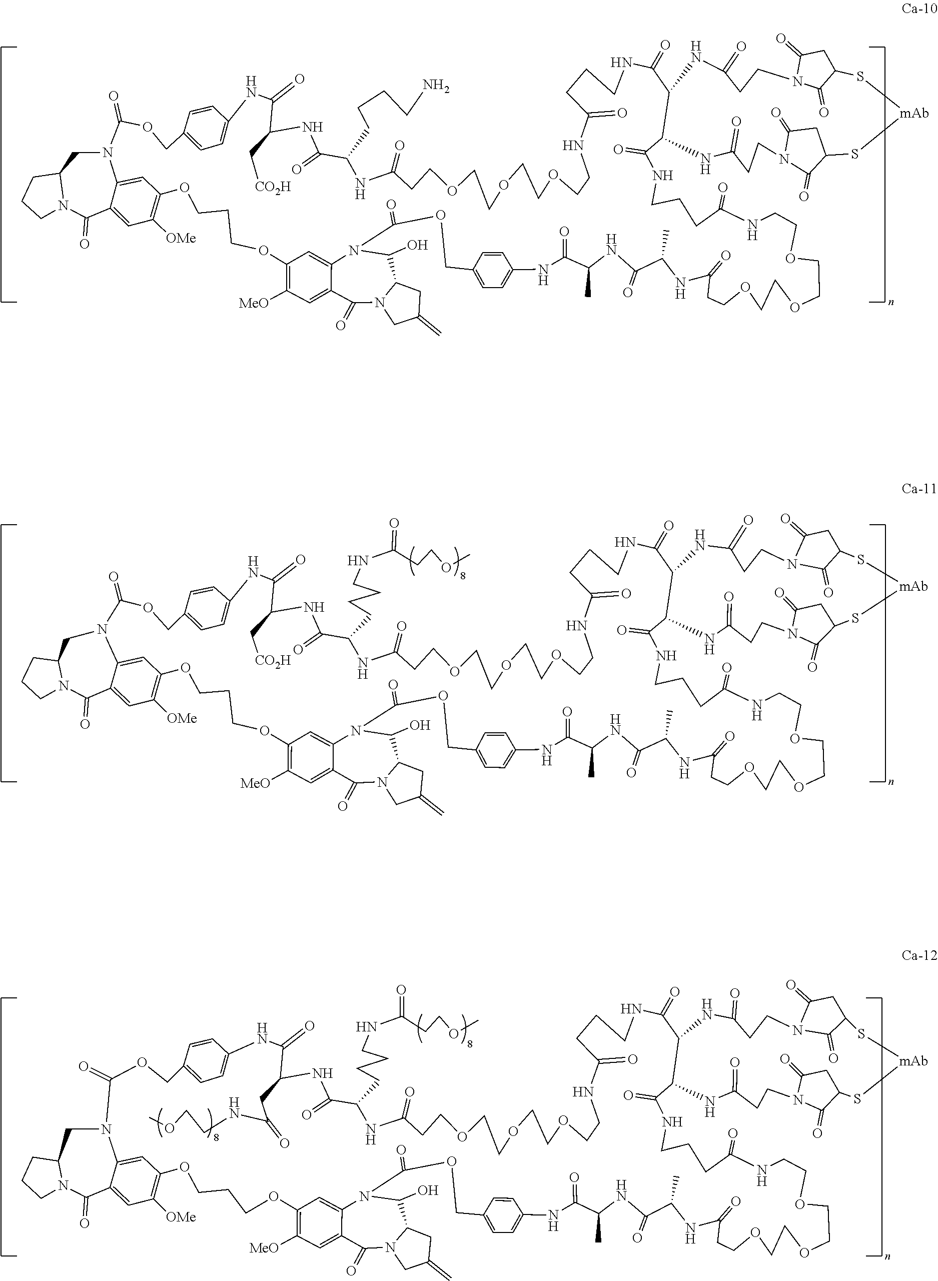 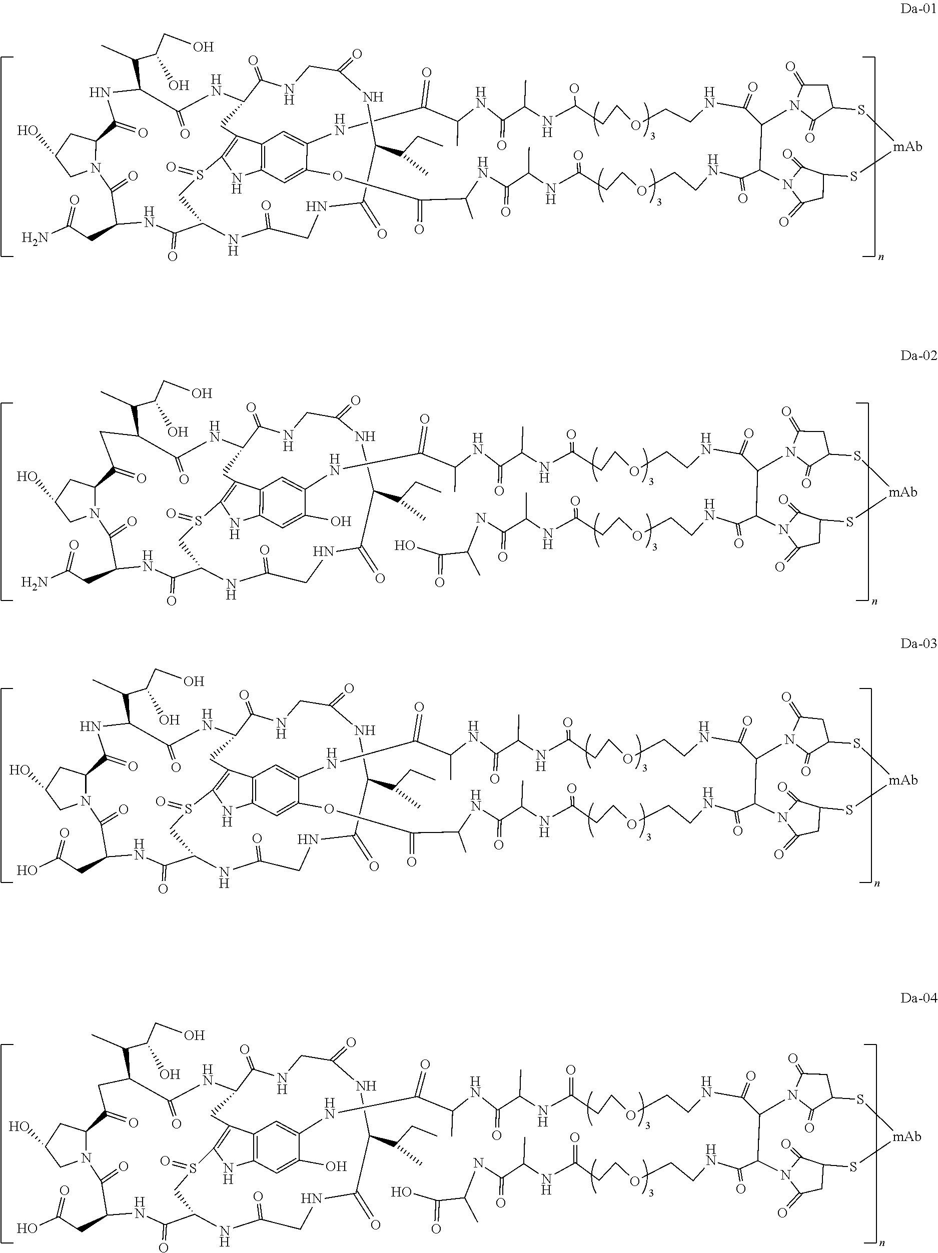 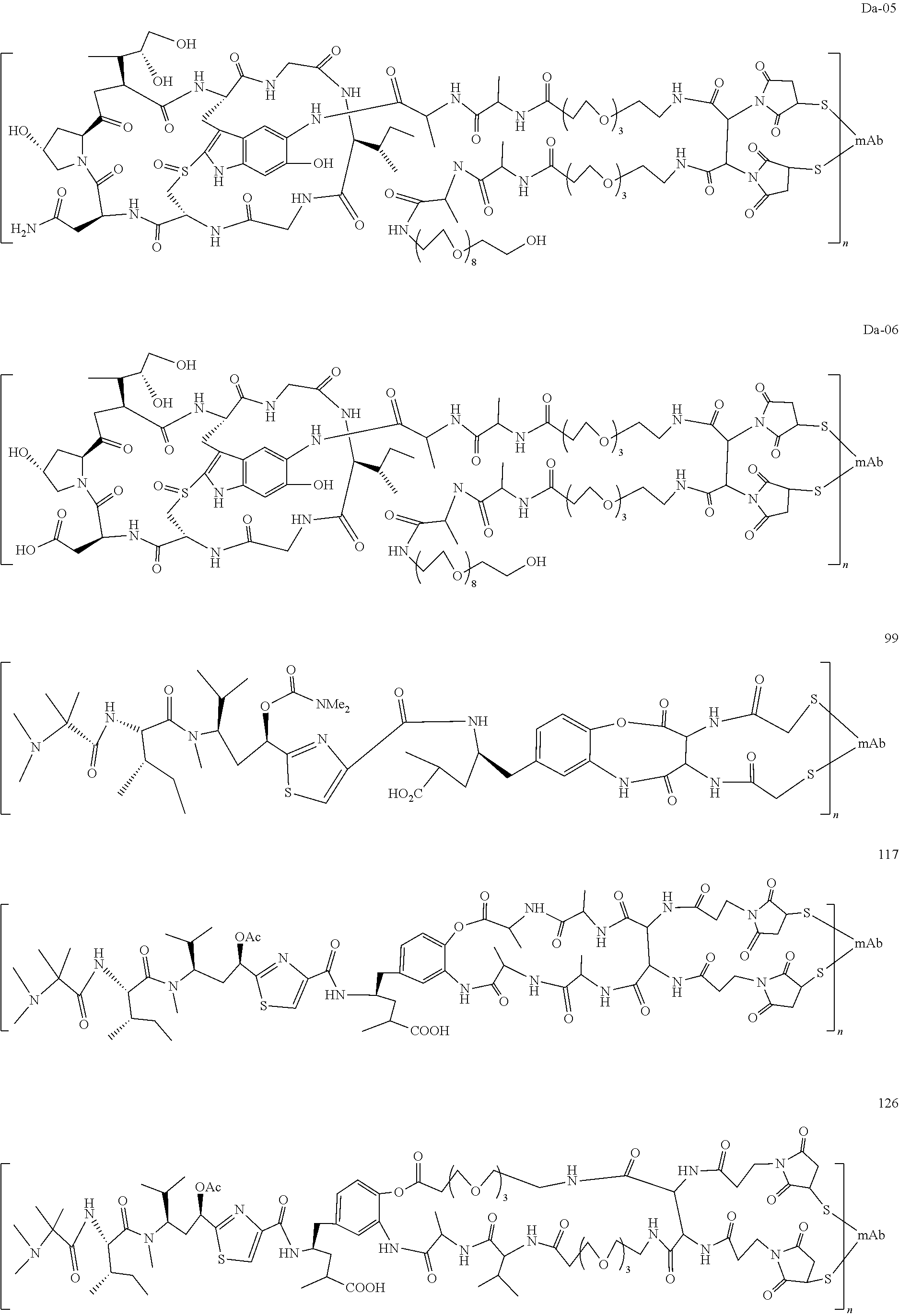 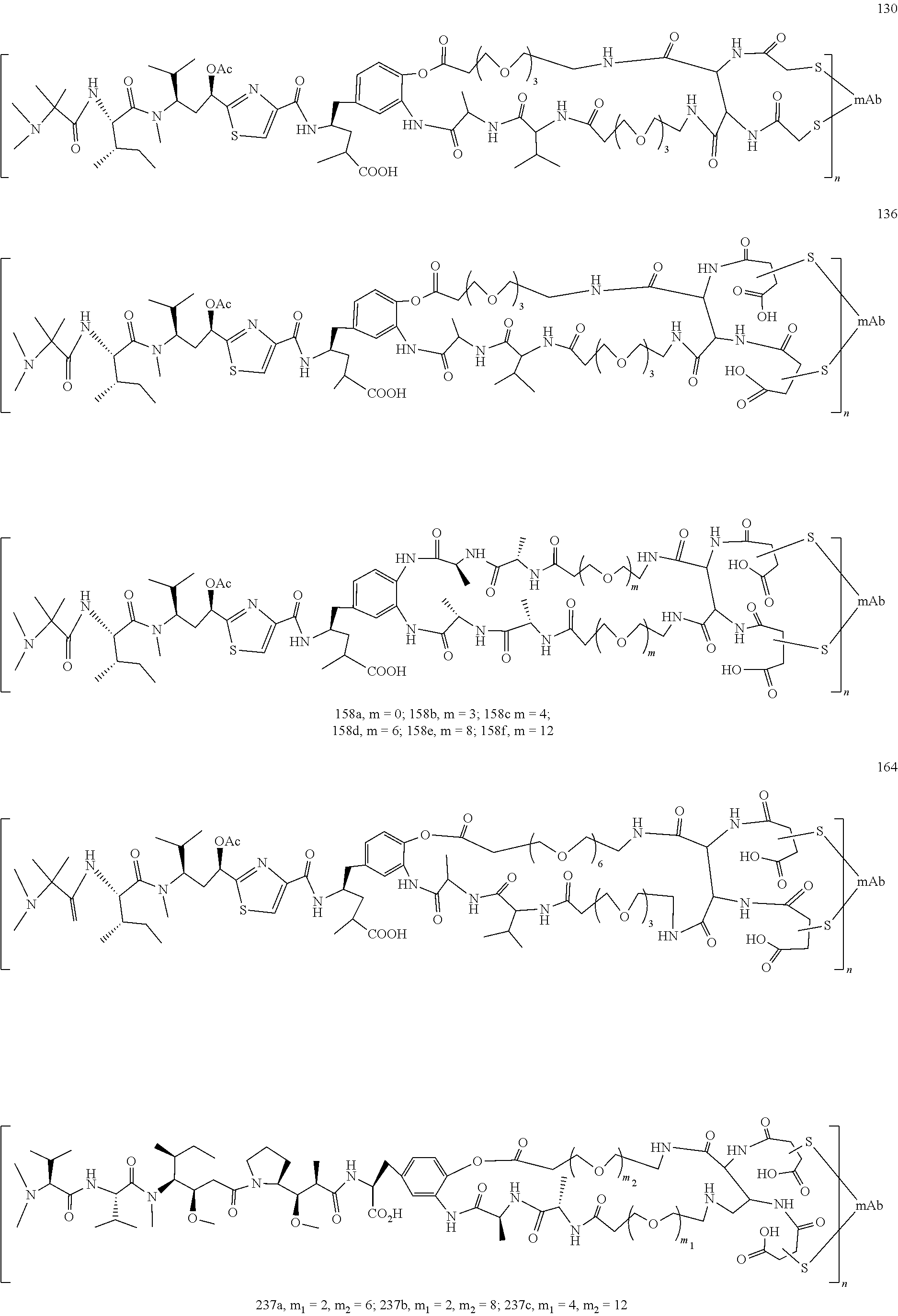 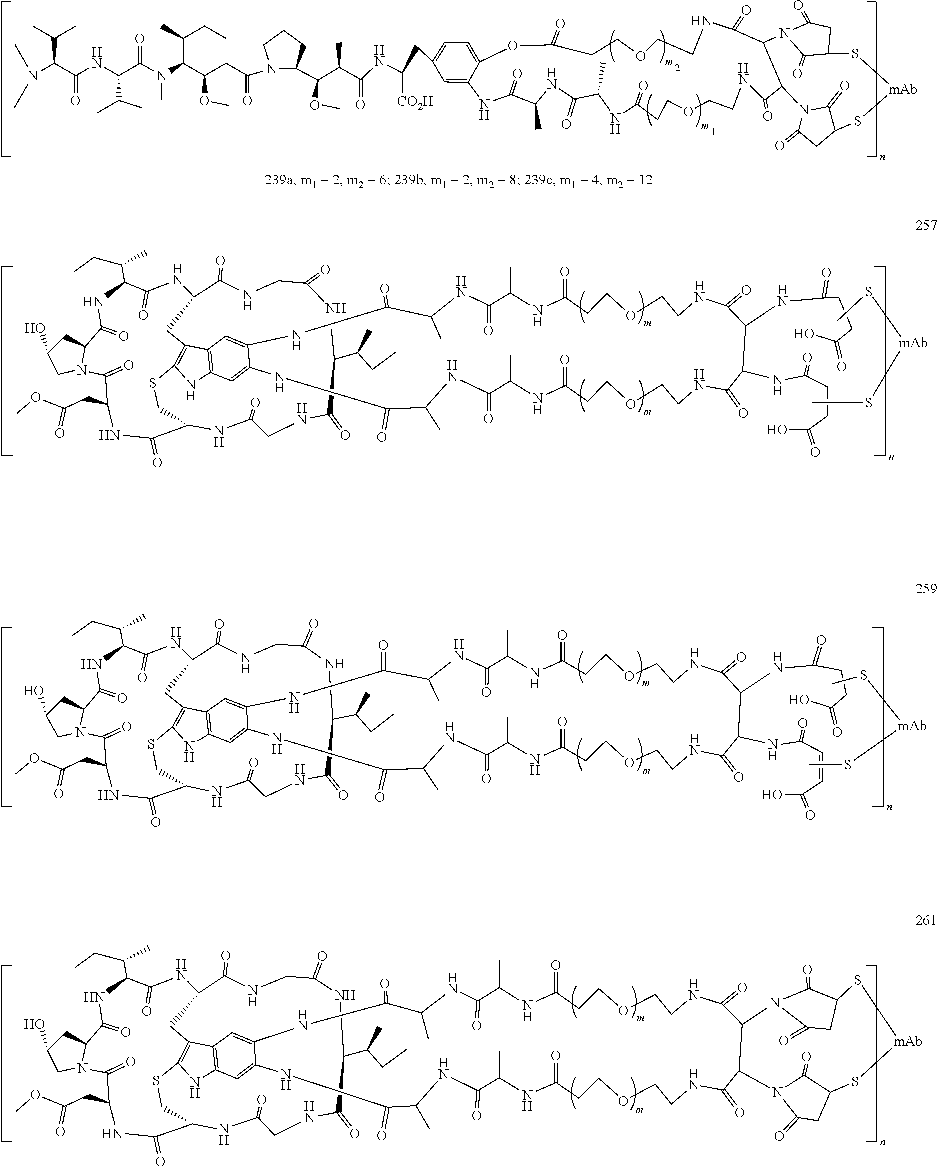 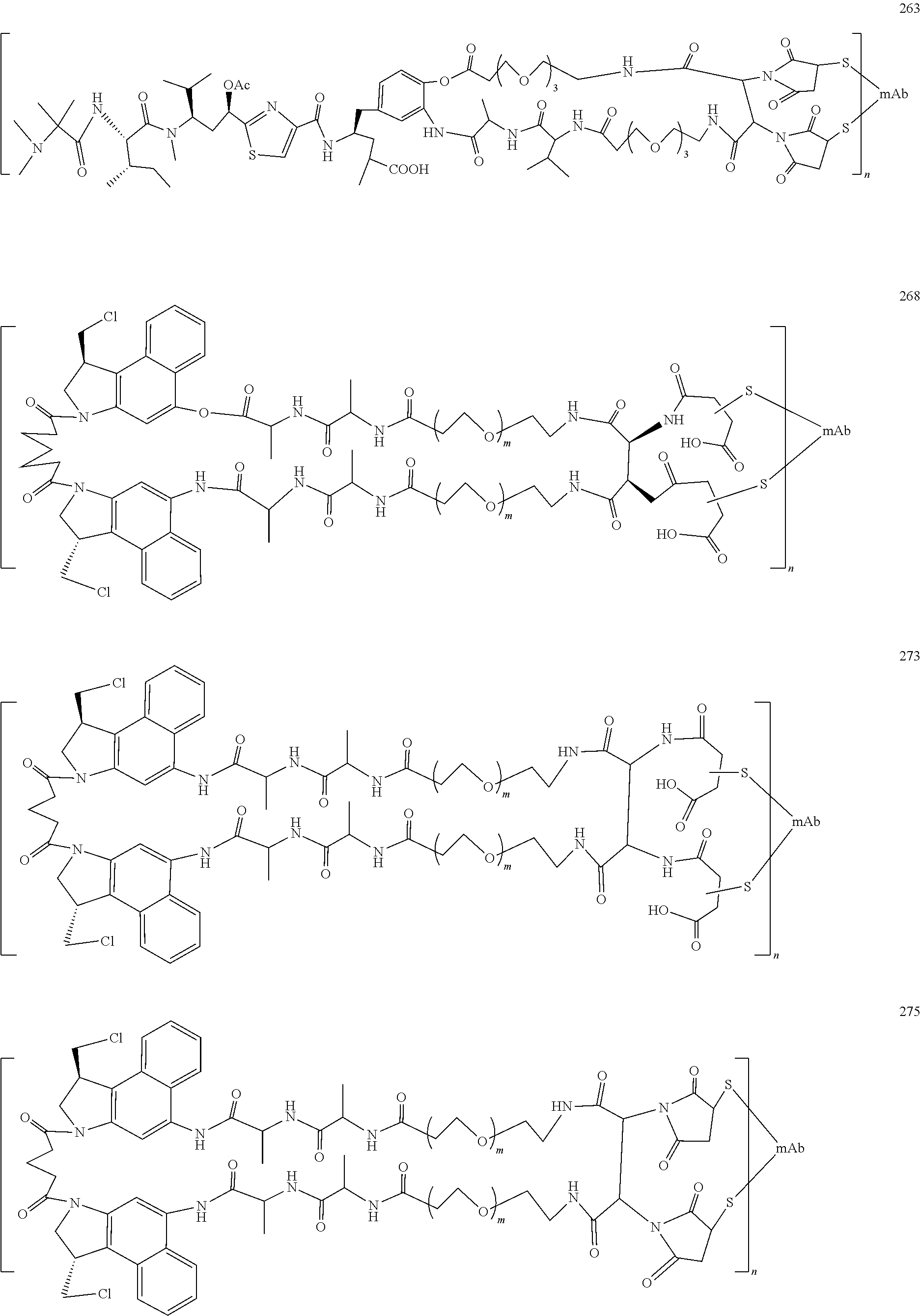 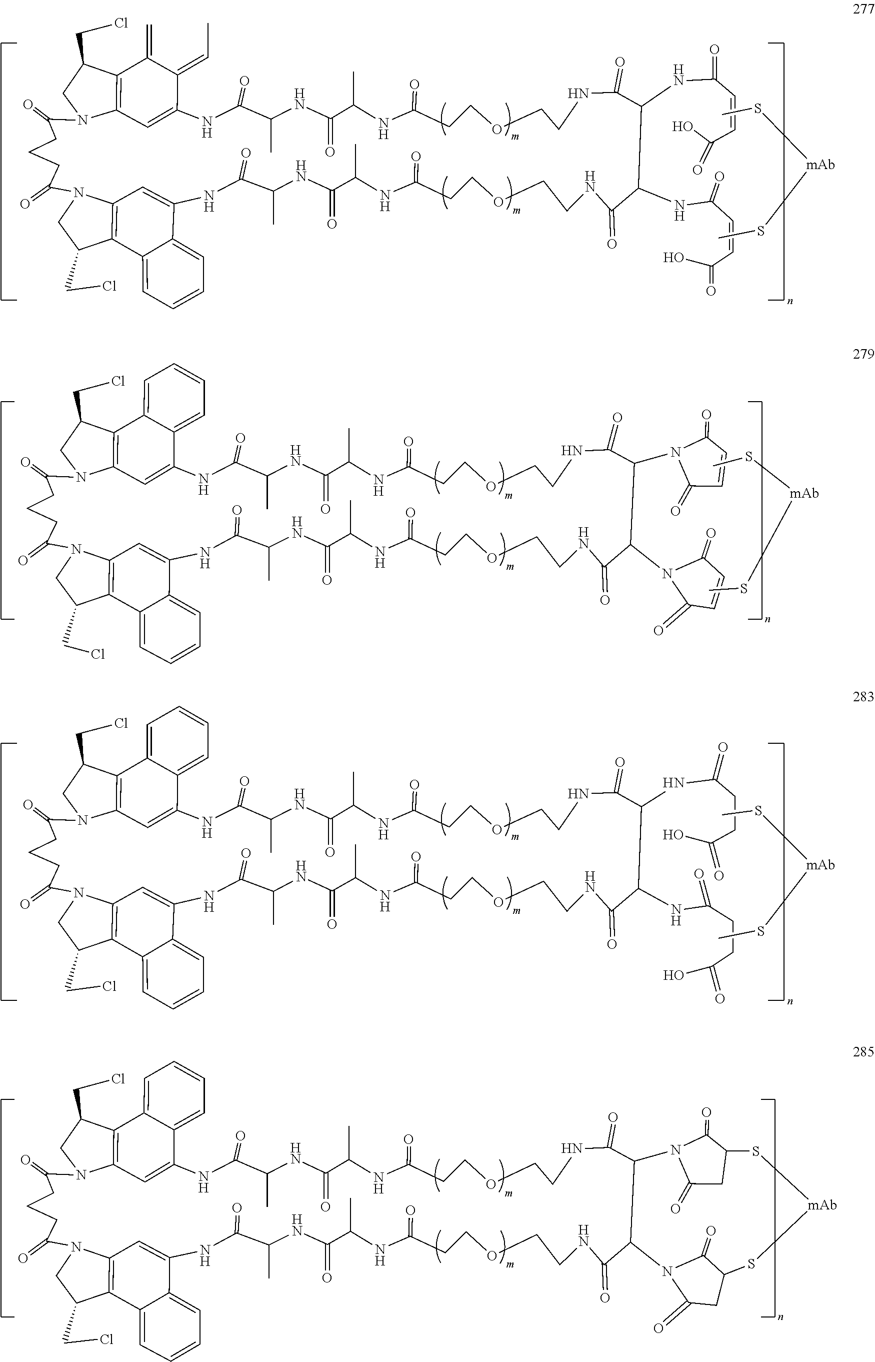 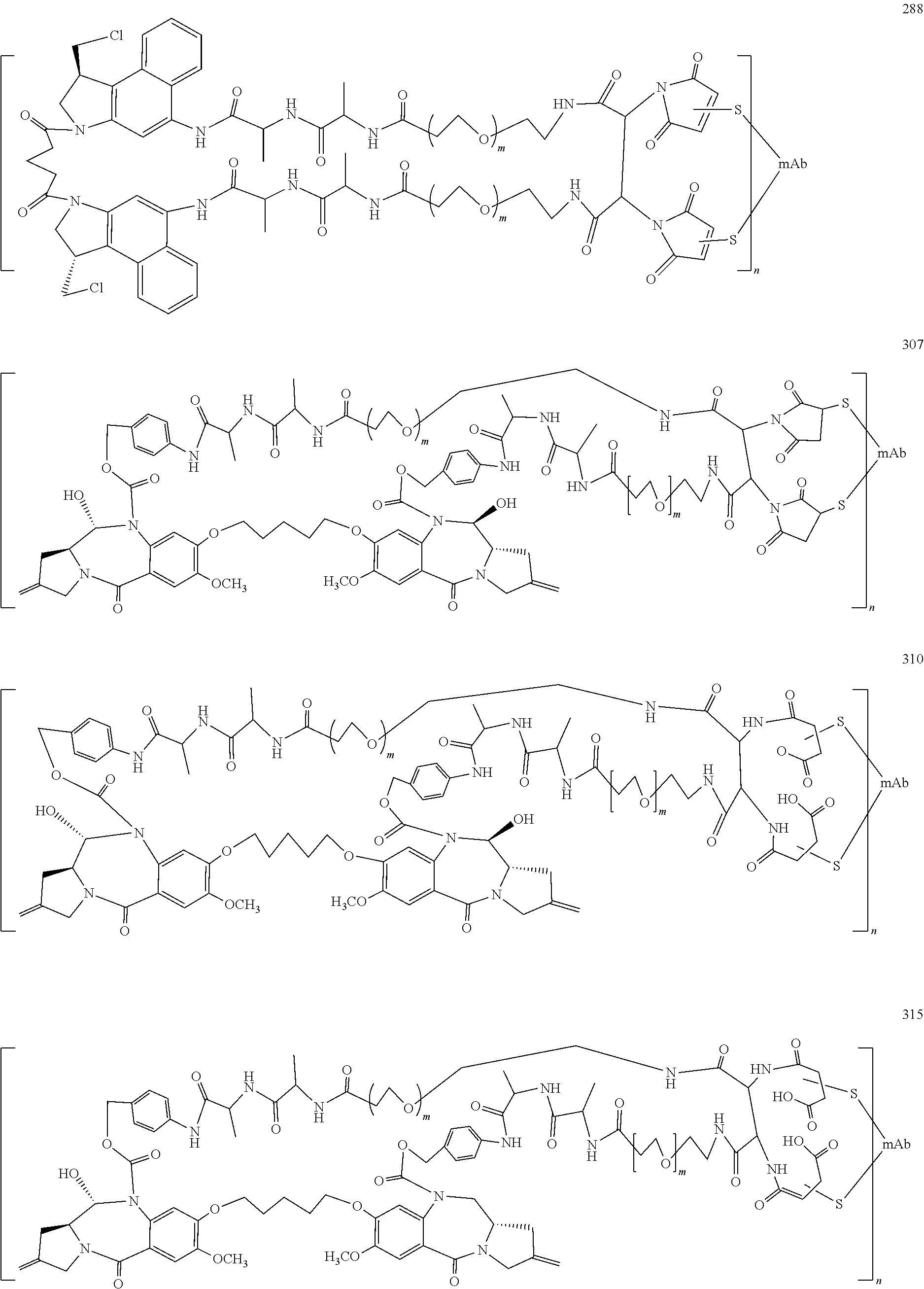 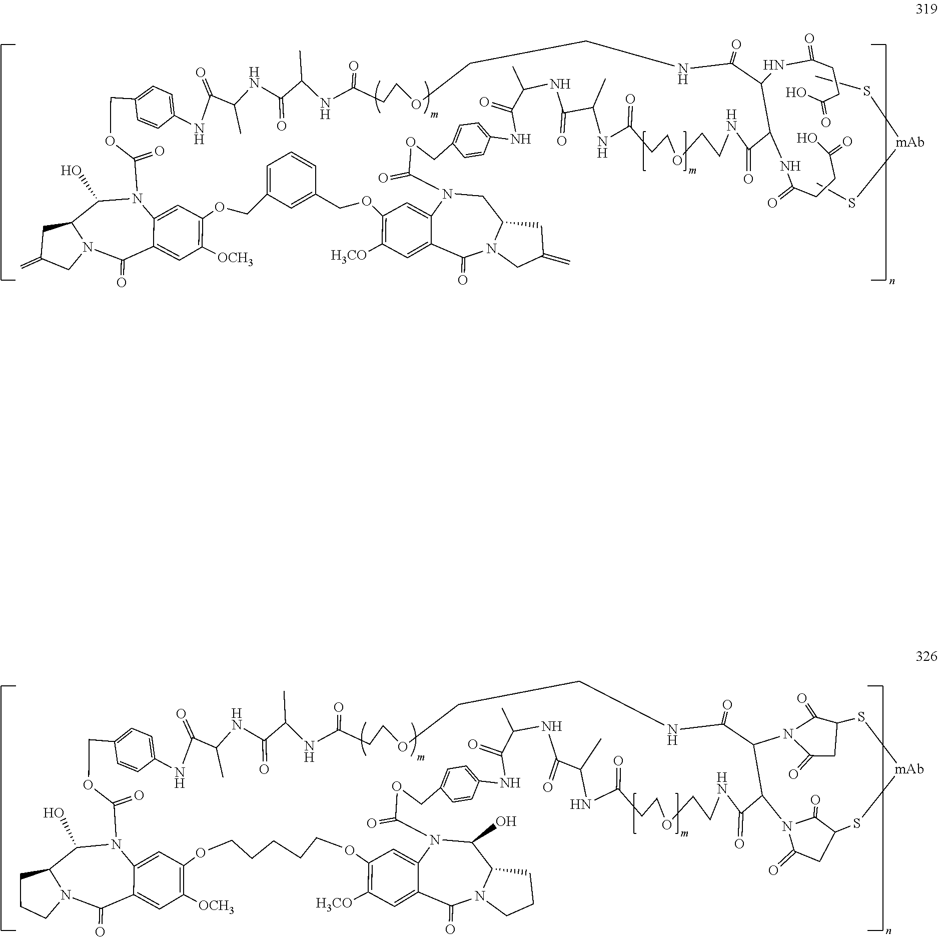
wherein m₁, and n are defined the same as in claim 48; mAb is an antibody; a cross bond means that it can connect either one of two atoms.

85. A pharmaceutical composition comprising a therapeutically effective amount of the conjugate compound of claim 48, and a pharmaceutically acceptable salt, carrier, diluent, or excipient therefore, or a combination of the conjugates thereof, for the treatment or prevention of a cancer, or an autoimmune disease, or an infectious disease.

86. The pharmaceutical composition according to claim 85, which is either in a liquid formula or in a formulated lyophilized solid, comprising by weight of: 0.01%-99% of one or more the conjugate compound, 0.0%-20.0% of one or more polyols; 0.0%-2.0% of one or more surfactants; 0.0%-5.0% of one or more preservatives; 0.0%-30% of one or more amino acids; 0.0%-5.0% of one or more antioxidants: 0.0%-0.3% of one or more metal chelating agents; 0.0%-30.0% of one or more buffer salts for adjusting pH of the formulation to pH 4.5 to 8.5; and 0.0%-30.0% of one or more of isotonic agent for adjusting osmotic pressure between about 250 to 350 mOsm when reconstituted for administration to a patient;
wherein the polyol is fructose, mannose, maltose, lactose, arabinose, xylose, ribose, rhamnose, galactose, glucose, sucrose, trehalose, sorbose, melezitose, raffinose, mannitol, xylitol, erythritol, maltitol, lactitol, erythritol, threitol, sorbitol, glycerol, or L-gluconate and its metallic salts);
wherein the surfactant is polysorbate 20, polysorbate 40, polysorbate 65, polysorbate 80, polysorbate 81, or polysorbate 85, poloxamer, poly(ethylene oxide)-poly(propylene oxide), polyethylene-polypropylene, Triton; sodium dodecyl sulfate (SDS), sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; or isostearyl ethylimidonium ethosulfate; polyethyl glycol, polypropyl glycol, and copolymers of ethylene and propylene glycol;
wherein the preservative is benzyl alcohol, octadecyldimethylbenzyl ammonium chloride, hexamethonium chloride, benzalkonium chloride, benzethonium chloride, phenol, butyl and benzyl alcohol, alkyl parabens, methyl or propyl paraben, catechol, resorcinol, cyclohexanol, 3-pentanol, or m-cresol;
wherein the amino acid is arginine, cystine, glycine, lysine, histidine, ornithine, isoleucine, leucine, alanine, glycine glutamic acid or aspartic acid;
wherein the antioxidant is ascorbic acid, glutathione, cystine or and methionine;
wherein the chelating agent is EDTA or EGTA;
wherein the buffer salt is sodium, potassium, ammonium, or trihydroxyethylamino salts of citric acid, ascorbic acid, gluconic acid, carbonic acid, tartaric acid, succinic acid, acetic acid or phthalic acid; Tris or tromethamine hydrochloride, phosphate or sulfate; arginine, glycine, glycylglycine, or histidine with anionic acetate, chloride, phosphate, sulfate, or succinate salt;
wherein the tonicity agent is mannitol, sorbitol, sodium acetate, potassium chloride, sodium phosphate, potassium phosphate, trisodium citrate, or sodium chloride.

87. The pharmaceutical composition according to claim 85, which is held in a vial, bottle, pre-filled syringe, or pre-filled auto-injector syringe, in a form of a liquid or lyophilized solid.

88. The conjugate compound of claim 48, having in vitro, in vivo or ex vivo cell killing activity.

89. A pharmaceutical composition according to claim 85, further comprising a chemotherapeutic agent, a radiation therapy agent, an immunotherapy agent, an autoimmune disorder agent, an anti-infectious agents or another conjugate for synergistically treatment or prevention of a cancer, or an autoimmune disease, or an infectious disease.

90. The pharmaceutical composition according to claim 89, wherein the another conjugate is one or several of the following drugs: Abatacept, abemaciclib, Abiraterone acetate, Abraxane, Aducanumab, Acetaminophen/hydrocodone, Acalabrutinib, aducanumab, Adalimumab, ADXS31-142, ADXS-HER2, afatinib dimaleate, aldesleukin, alectinib, alemtuzumab, allitinib, Alitretinoin, ado-trastuzumab emtansine, Amphetamine/dextroamphetamine, anastrozole, apatinib, Aripiprazole, anthracyclines, Aripiprazole, Atazanavir, Atezolizumab, Atorvastatin, Avelumab, AVXS-101, Axicabtagene ciloleucel, axitinib, belinostat, BCG Live, Bevacizumab, bexarotene, blinatumomab, Bortezomib, bosutinib, brentuximab vedotin, brigatinib, Brolucizumab, Budesonide, Budesonide/formoterol, Buprenorphine, BYL719 (alpha-specific PI3K inhibitor), Cabazitaxel, Cabozantinib, capmatinib, Capecitabine, carfilzomib, chimeric antigen receptor-engineered T (CAR-T) cells, Celecoxib, ceritinib, Cetuximab, chiauranib, Chidamide, Ciclosporin, Cinacalcet, crizotinib, Cobimetinib, Cosentyx, crizotinib, Tisagenlecleucel, Dabigatran, dabrafenib, dacarbazine, daclizumab, dacomotinib, daptomycin, Daratumumab, Darbepoetin alfa, Darunavir, dasatinib, denileukin diftitox, Denosumab, Depakote, Dexlansoprazole, Dexmethylphenidate, Dexamethasone, DigniCap Cooling System, L-3,4-dihydroxyphenyl-alanine, Dinutuximab, dornase alfa, Doxycycline, Duloxetine, Duvelisib, durvalumab, elotuzumab, emicizumab, Emtricibine/Rilpivirine/Tenofovir, disoproxil fumarate, Emtricitbine/tenofovir/efavirenz, Enoxaparin, ensartinib, Enzalutamide, epitinib, Epoetin alfa, erlotinib, Esomeprazole, Eszopiclone, Etanercept, Everolimus, exemestane, everolimus, exenatide ER, Ezetimibe, Ezetimibe/simvastatin, famitinib, Fenofibrate, Filgotinib, Filgrastim, fingolimod, flumatinib, Fluticasone propionate, Fluticasone/salmeterol, fruquintinib, fulvestrant, gazyva, gefitinib, Glatiramer, Goserelin acetate, GSK2857916 (BCMA-ADC), henatinib, Icotinib, Imatinib, Ibritumomab tiuxetan, ibrutinib, icotinib, idelalisib, ifosfamide, Infliximab, imiquimod, ImmuCyst, Immuno BCG, iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, lapatinib, Ipilimumab, Ipratropium bromide/salbutamol, Ixazomib, Kanuma, Lanadelumab, Lanreotide acetate, lenalidomide, lenaliomide, lenvatinib mesylate, letrozole, Levothyroxine, Levothyroxine, Lidocaine, Linezolid, Liraglutide, Lisdexamfetamine, LN-144 (tumor-infiltrating lymphocyte), lorlatinib, lucitanib/delitinib, Memantine, Methoxy polyethylene glycol-epoetin beta, Methylphenidate, Metoprolol, Mekinist, mericitabine/Rilpivirine/Tenofovir, Modafinil, Mometasone, Mycidac-C, mycophenolic acid, Necitumumab, neratinib, Nilotinib, niraparib, Nivolumab, ofatumumab, obinutuzumab, ocrelizumab, olaparib, Olmesartan, Olmesartan/hydrochlorothiazide, Omalizumab, Omega-3 fatty acid ethyl esters, Oncorine, Oseltamivir, Osimertinib, Oxycodone, Ozanimod, palbociclib, Palivizumab, panitumumab, panobinostat, pazopanib, pembrolizumab, PD-1 antibody, PD-L1 antibody, Pemetrexed, pertuzumab, Pirfenidone, Pneumococcal conjugate vaccine, pomalidomide, Pregabalin, ProscaVax, Propranolol, puquitinib, pyrotinib, Quetiapine, Rabeprazole, radium 223 chloride, Raloxifene, Raltegravir, ramucirumab, Ranibizumab, regorafenib, ribociclib, Risankizumab, Rituximab, Rivaroxaban, romidepsin, Rosuvastatin, ruxolitinib phosphate, Salbutamol, savolitinib, semaglutide, Sevelamer, Sildenafil, siltuximab, simotinib, sipatinib/cipatinib, Siponimod, Sipuleucel-T, Sitagliptin, Sitagliptin/metformin, Solifenacin, solanezumab, Sonidegib, Sorafenib, sulfatinib, Sunitinib, tacrolimus, tacrimus, Tadalafil, tamoxifen, Tafinlar, Talimogene laherparepvec, talazoparib, Telaprevir, talazoparib, Temozolomide, temsirolimus, Tenecteplase, Tenofovir/emtricitabine, tenofovir disoproxil fumarate, Testosterone gel, tezacaftor/ivacaftor, Thalidomide, theliatinib, TICE BCG, Tiotropium bromide, Tisagenlecleucel, Tocilizumab, toremifene, trametinib, Trastuzumab, Trabectedin (ecteinascidin 743), trametinib, tremelimumab, Trifluridine/tipiracil, Tretinoin, Upadacitinib, Uro-BCG, Ustekinumab, Valoctocogene roxaparvovec, Valsartan, veliparib, vandetanib, vemurafenib, venetoclax, vismodegib, volitinib, vorinostat, ziv-aflibercept, Zostavax, and their analogs, derivatives, pharmaceutically acceptable salts, carriers, diluents, or excipients thereof, or a combination above thereof.
